(19) United States
(12) Patent Application Publication
(10) Pub. No.: US 2025/0204242 A1
(43) Pub. Date: Jun. 19, 2025

(54) COMPOUND, MATERIAL FOR ORGANIC ELECTROLUMINESCENCE DEVICE, ORGANIC ELECTROLUMINESCENCE DEVICE, AND ELECTRONIC APPARATUS

(71) Applicant: IDEMITSU KOSAN CO., LTD., Tokyo (JP)

(72) Inventors: Masato MITANI, Tokyo (JP); Sayaka MIZUTANI, Tokyo (JP); Ryota TAKAHASHI, Tokyo (JP); Keitaro YAMADA, Tokyo (JP)

(73) Assignee: IDEMITSU KOSAN CO., LTD., Tokyo (JP)

(21) Appl. No.: 18/851,825

(22) PCT Filed: Jun. 29, 2023
(86) PCT No.: PCT/JP2023/024220

§ 371 Date: Sep. 27, 2024

(30) Foreign Application Priority Data
Jul. 13, 2022 (JP) 2022-112508

(51) Int. Cl. H10K 85/60; C07D 239/26; C07D 405/10; H10K 50/16

(52) U.S. Cl. CPC H10K 85/622; C07D 239/26; C07D 405/10; H10K 85/626; H10K 85/654; H10K 85/6574; H10K 50/16

(57) ABSTRACT

A compound represented by the following formula (1):

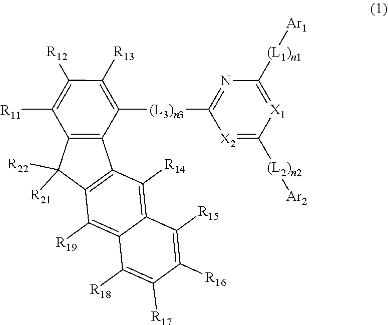

### TECHNICAL FIELD

The invention relates to a novel compound, a material for an organic electroluminescence device, an organic electroluminescence device, and an electronic apparatus.

### BACKGROUND ART

When a voltage is applied to organic electroluminescence device (hereinafter, also referred to as organic EL device), holes and electrons are injected into an emitting layer from an anode and cathode, respectively. Then, the injected holes and electrons are recombined in the emitting layer, and excitons are formed therein.

Conventional organic EL devices have not yet achieved satisfactory device performance. In order to improve the device performance, material used in organic EL device has been gradually improved, but further enhancement of the performance is required.

### RELATED ART DOCUMENTS

### Patent Documents

[Patent Document 1] WO 2016/105141
[Patent Document 2] WO 2022/181157

### SUMMARY OF THE INVENTION

It is an object of the invention is to provide a high-performance organic EL device and a compound that can realize such an organic EL device.

As a result of intensive studies to achieve the above-described object, the inventors have found that a compound having a particular configuration contributes to increase of the performance of organic EL devices, and have completed the invention.

According to the invention, the following compound and so on are provided.

1. A compound represented by the following formula (1):

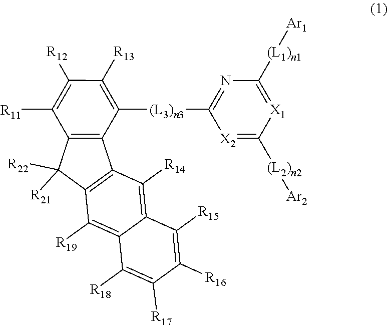

Wherein in the formula (1),
X₁ and X₂ are independently N or CH, and any one of X₁ and X₂ is N,
Ar₁ and Ar₂ are independently
a substituted or unsubstituted aryl group including 6 to 50 ring carbon atoms, or
a substituted or unsubstituted monovalent heterocyclic group including 5 to 50 ring atoms,
L₁ to L₃ are independently
a single bond,
a substituted or unsubstituted arylene group including 6 to 50 ring carbon atoms, or
a substituted or unsubstituted divalent heterocyclic group including 5 to 50 ring atoms,
when n1 is an integer of 0 to 4 and n1 is 0, (L₁)ₙ₁ is a single bond,
when a plurality of L₁'s is present, the plurality of L₁'s is the same as or different from each other,
when n2 is an integer of 0 to 4 and n2 is 0, (L₂)ₙ₂ is a single bond,
when a plurality of L₂'s is present, the plurality of L₂'s is the same as or different from each other,
when n3 is an integer of 0 to 4 and n3 is 0, (L₃)ₙ₃ is a single bond,
when a plurality of L₃'s is present, the plurality of L₃'s is the same as or different from each other,
R₁₁ to R¹⁹ are independently a hydrogen atom or a substituent R,
a set of adjacent two or more of R₁₁ to R¹⁹ do not bond each other,
R₂₁ and R₂₂ form a substituted or unsubstituted saturated or unsaturated ring by bonding with each other or do not form a substituted or unsubstituted saturated or unsaturated ring,
R₂₁ and R₂₂ which do not form substituted or unsubstituted saturated or unsaturated ring are independently, a hydrogen atom, or a substituted or unsubstituted alkyl group including 1 to 50 carbon atoms,
the substituent R is selected from the group consisting of,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms:
wherein R₉₀₁ to R₉₀₇ are independently:
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms, and
when two or more of each of R₉₀₁ to R₉₀₇ are present, the two or more of each of R₉₀₁ to R₉₀₇ may be the same as or different from each other, and
when two or more substituent R's are present, the two or more substituent R's may be the same as or different from each other.

2. An organic electroluminescence device comprising
a cathode;
an anode; and
one or two or more organic layers arranged between the cathode and the anode,
wherein at least one of organic layer comprises the compound according to 1.

3. An electronic apparatus comprising organic electroluminescence device according to 2.

According to the invention, it is possible to provide a high-performance organic EL device and a compound that can realize the organic EL device.

### BRIEF DESCRIPTION OF THE DRAWINGS

FIG. 1 is a view for the schematic configuration of an organic EL device according to one aspect of the invention.

### MODE FOR CARRYING OUT THE INVENTION

### Definition

In this specification, a hydrogen atom includes its isotopes different in the number of neutrons, namely, a protium, a deuterium and a tritium.

In this specification, at a bondable position in a chemical formula where a symbol such as “R”, or “D” representing a deuterium atom is not indicated, a hydrogen atom, that is, a protium atom, a deuterium atom or a tritium atom is bonded.

In this specification, the number of ring carbon atoms represents the number of carbon atoms forming a subject ring itself among the carbon atoms of a compound having a structure in which atoms are bonded in a ring form (for example, a monocyclic compound, a fused ring compound, a cross-linked compound, a carbocyclic compound, or a heterocyclic compound). When the subject ring is substituted by a substituent, the carbon contained in the substituent is not included in the number of ring carbon atoms. The same shall apply to “the number of ring carbon atoms” described below, unless otherwise specified. For example, a benzene ring has 6 ring carbon atoms, a naphthalene ring includes 10 ring carbon atoms, a pyridine ring includes 5 ring carbon atoms, and a furan ring includes 4 ring carbon atoms. Further, for example, a 9,9-diphenylfluorenyl group includes 13 ring carbon atoms, and a 9,9′-spirobifluorenyl group includes 25 ring carbon atoms.

When a benzene ring is substituted by, for example, an alkyl group as a substituent, the number of carbon atoms of the alkyl group is not included in the number of ring carbon atoms of the benzene ring. Therefore, the number of ring carbon atoms of the benzene ring substituted by the alkyl group is 6. When a naphthalene ring is substituted by, for example, an alkyl group as a substituent, the number of carbon atoms of the alkyl group is not included in the number of ring carbon atoms of the naphthalene ring. Therefore, the number of ring carbon atoms of the naphthalene ring substituted by the alkyl group is 10.

In this specification, the number of ring atoms represents the number of atoms forming a subject ring itself among the atoms of a compound having a structure in which atoms are bonded in a ring form (for example, the structure includes a monocyclic ring, a fused ring and a ring assembly) (for example, a monocyclic compound, a fused ring compound, a cross-linked compound, a carbocyclic compound and a heterocyclic compound). The number of ring atoms does not include atoms which do not form the ring (for example, a hydrogen atom which terminates a bond of the atoms forming the ring), or atoms contained in a substituent when the ring is substituted by the substituent. The same shall apply to “the number of ring atoms” described below, unless otherwise specified. For example, the number of atoms of a pyridine ring is 6, the number of atoms of a quinazoline ring is 10, and the number of a furan ring is 5. For example, hydrogen atoms bonded to a pyridine ring and atoms constituting a substituent substituted on the pyridine ring are not included in the number of ring atoms of the pyridine ring. Therefore, the number of ring atoms of a pyridine ring with which a hydrogen atom or a substituent is bonded is 6. For example, hydrogen atoms and atoms constituting a substituent which are bonded with a quinazoline ring is not included in the number of ring atoms of the quinazoline ring. Therefore, the number of ring atoms of a quinazoline ring with which a hydrogen atom or a substituent is bonded is 10.

In this specification, “XX to YY carbon atoms” in the expression “a substituted or unsubstituted ZZ group including XX to YY carbon atoms” represents the number of carbon atoms in the case where the ZZ group is unsubstituted by a substituent, and does not include the number of carbon atoms of a substituent in the case where the ZZ group is substituted by the substituent. Here, “YY” is larger than “XX”, and “XX” means an integer of 1 or more and “YY” means an integer of 2 or more.

In this specification, “XX to YY atoms” in the expression “a substituted or unsubstituted ZZ group including XX to YY atoms” represents the number of atoms in the case where the ZZ group is unsubstituted by a substituent, and does not include the number of atoms of a substituent in the case where the ZZ group is substituted by the substituent. Here, “YY” is larger than “XX”, and “XX” means an integer of 1 or more and “YY” means an integer of 2 or more.

In this specification, the unsubstituted ZZ group represents the case where the “substituted or unsubstituted ZZ group” is a “ZZ group unsubstituted by a substituent”, and the substituted ZZ group represents the case where the “substituted or unsubstituted ZZ group“is a” ZZ group substituted by a substituent”.

In this specification, a term “unsubstituted” in the case of “a substituted or unsubstituted ZZ group” means that hydrogen atoms in the ZZ group are not substituted by a substituent. Hydrogen atoms in a term “unsubstituted ZZ group” are a protium atom, a deuterium atom, or a tritium atom.

In this specification, a term “substituted” in the case of “a substituted or unsubstituted ZZ group” means that one or more hydrogen atoms in the ZZ group are substituted by a substituent. Similarly, a term “substituted” in the case of “a BB group substituted by an AA group” means that one or more hydrogen atoms in the BB group are substituted by the AA group.

“Substituent as Described in this Specification”

Hereinafter, the substituent described in this specification will be explained.

The number of ring carbon atoms of the “unsubstituted aryl group” described in this specification is 6 to 50, preferably 6 to 30, and more preferably 6 to 18, unless otherwise specified.

The number of ring atoms of the “unsubstituted heterocyclic group” described in this specification is 5 to 50, preferably 5 to 30, and more preferably 5 to 18, unless otherwise specified.

The number of carbon atoms of the “unsubstituted alkyl group” described in this specification is 1 to 50, preferably 1 to 20, and more preferably 1 to 6, unless otherwise specified.

The number of carbon atoms of the “unsubstituted alkenyl group” described in this specification is 2 to 50, preferably 2 to 20, and more preferably 2 to 6, unless otherwise specified.

The number of carbon atoms of the “unsubstituted alkynyl group” described in this specification is 2 to 50, preferably 2 to 20, and more preferably 2 to 6, unless otherwise specified.

The number of ring carbon atoms of the “unsubstituted cycloalkyl group” described in this specification is 3 to 50, preferably 3 to 20, and more preferably 3 to 6, unless otherwise specified.

The number of ring carbon atoms of the “unsubstituted arylene group” described in this specification is 6 to 50, preferably 6 to 30, and more preferably 6 to 18, unless otherwise specified.

The number of ring atoms of the “unsubstituted divalent heterocyclic group” described in this specification is 5 to 50, preferably 5 to 30, and more preferably 5 to 18, unless otherwise specified.

The number of carbon atoms of the “unsubstituted alkylene group” described in this specification is 1 to 50, preferably 1 to 20, and more preferably 1 to 6, unless otherwise specified.

### “Substituted or Unsubstituted Aryl Group”

Specific examples of the “substituted or unsubstituted aryl group” described in this specification (specific example group G1) include the following unsubstituted aryl groups (specific example group G1A), substituted aryl groups (specific example group G1B), and the like. (Here, the unsubstituted aryl group refers to the case where the “substituted or unsubstituted aryl group” is an “aryl group unsubstituted by a substituent”, and the substituted aryl group refers to the case where the “substituted or unsubstituted aryl group“is an” aryl group substituted by a substituent”.). In this specification, in the case where simply referred as an “aryl group”, it includes both a “unsubstituted aryl group” and a “substituted aryl group.”

The “substituted aryl group” means a group in which one or more hydrogen atoms of the “unsubstituted aryl group” are substituted by a substituent. Specific examples of the “substituted aryl group” include, for example, groups in which one or more hydrogen atoms of the “unsubstituted aryl group” of the following specific example group G1A are substituted by a substituent, the substituted aryl groups of the following specific example group G1B, and the like. It should be noted that the examples of the “unsubstituted aryl group” and the examples of the “substituted aryl group” enumerated in this specification are mere examples, and the “substituted aryl group” described in this specification also includes a group in which a hydrogen atom bonded with a carbon atom of the aryl group itself in the “substituted aryl group” of the following specific group G1B is further substituted by a substituent, and a group in which a hydrogen atom of a substituent in the “substituted aryl group” of the following specific group G1B is further substituted by a substituent.

### Unsubstituted Aryl Group (Specific Example Group G1A):

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenalenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
a monovalent aryl group derived by removing one hydrogen atom from the ring structures represented by each of the following general formulas (TEMP-1) to (TEMP-15).

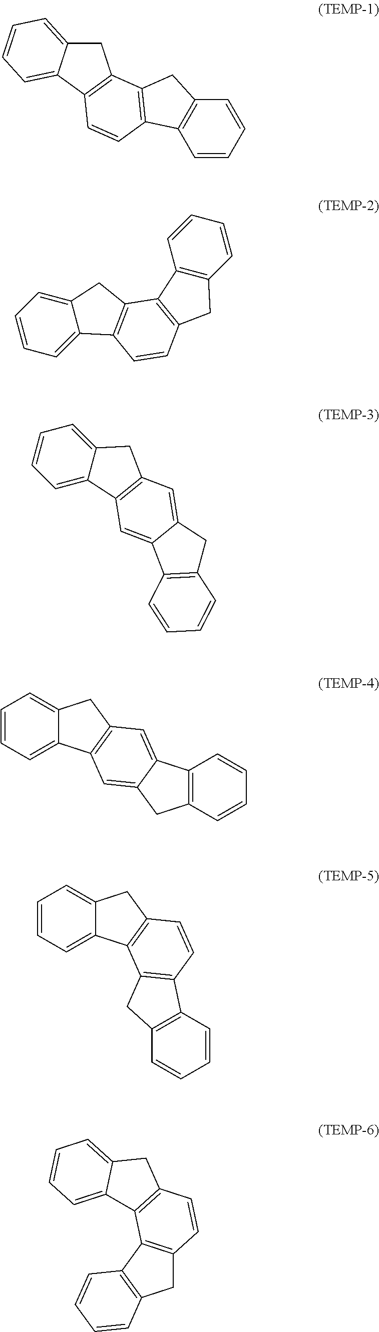

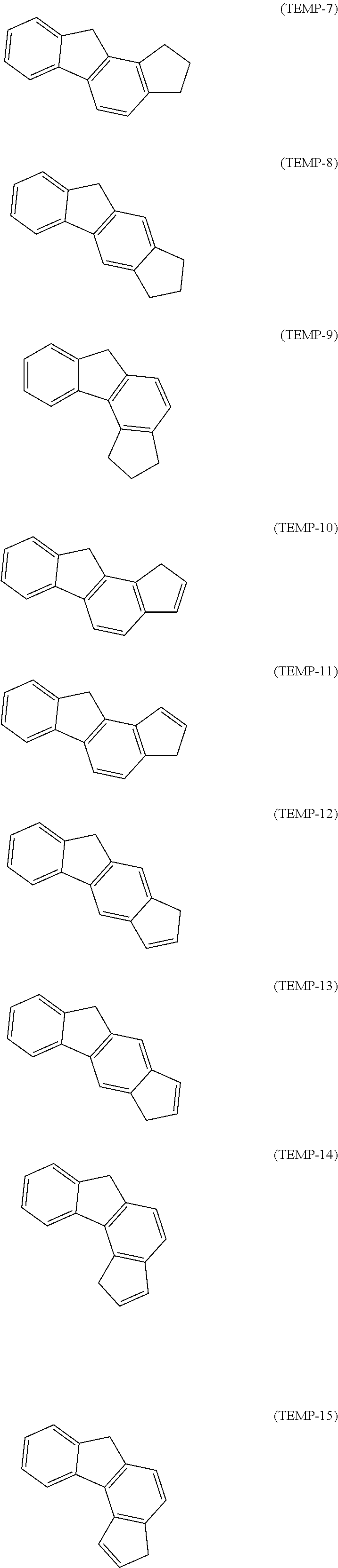

### Substituted Aryl Group (Specific Example Group G1B):

an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
an o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
a group in which one or more hydrogen atoms of a monovalent group derived from the ring structures represented by each of the general formulas (TEMP-1) to (TEMP-15) are substituted by a substituent.

### “Substituted or Unsubstituted Heterocyclic Group”

The “heterocyclic group” described in this specification is a ring group having at least one hetero atom in the ring atom. Specific examples of the hetero atom include a nitrogen atom, an oxygen atom, a sulfur atom, a silicon atom, a phosphorus atom, and a boron atom.

The “heterocyclic group” in this specification is a monocyclic group or a fused ring group.

The “heterocyclic group” in this specification is an aromatic heterocyclic group or a non-aromatic heterocyclic group.

Specific examples of the “substituted or unsubstituted heterocyclic group” (specific example group G2) described in this specification include the following unsubstituted heterocyclic group (specific example group G2A), the following substituted heterocyclic group (specific example group G2B), and the like. (Here, the unsubstituted heterocyclic group refers to the case where the “substituted or unsubstituted heterocyclic group“is a” heterocyclic group unsubstituted by a substituent”, and the substituted heterocyclic group refers to the case where the “substituted or unsubstituted heterocyclic group“is a” heterocyclic group substituted by a substituent”.). In this specification, in the case where simply referred as a “heterocyclic group”, it includes both the “unsubstituted heterocyclic group” and the “substituted heterocyclic group.”

The “substituted heterocyclic group” means a group in which one or more hydrogen atom of the “unsubstituted heterocyclic group” are substituted by a substituent. Specific examples of the “substituted heterocyclic group” include a group in which a hydrogen atom of “unsubstituted heterocyclic group” of the following specific example group G2A is substituted by a substituent, the substituted heterocyclic groups of the following specific example group G2B, and the like. It should be noted that the examples of the “unsubstituted heterocyclic group” and the examples of the “substituted heterocyclic group” enumerated in this specification are mere examples, and the “substituted heterocyclic group” described in this specification includes groups in which hydrogen atom bonded with a ring atom of the heterocyclic group itself in the “substituted heterocyclic group” of the specific example group G2B is further substituted by a substituent, and a group in which hydrogen atom of a substituent in the “substituted heterocyclic group” of the specific example group G2B is further substituted by a substituent.

Specific example group G2A includes, for example, the following unsubstituted heterocyclic group containing a nitrogen atom (specific example group G2A1), the following unsubstituted heterocyclic group containing an oxygen atom (specific example group G2A2), the following unsubstituted heterocyclic group containing a sulfur atom (specific example group G2A3), and the monovalent heterocyclic group derived by removing one hydrogen atom from the ring structures represented by each of the following general formulas (TEMP-16) to (TEMP-33) (specific example group G2A4).

Specific example group G2B includes, for example, the following substituted heterocyclic group containing a nitrogen atom (specific example group G2B1), the following substituted heterocyclic group containing an oxygen atom (specific example group G2B2), the following substituted heterocyclic group containing a sulfur atom (specific example group G2B3), and the following group in which one or more hydrogen atoms of the monovalent heterocyclic group derived from the ring structures represented by each of the following general formulas (TEMP-16) to (TEMP-33) are substituted by a substituent (specific example group G2B4).

### Unsubstituted Heterocyclic Group Containing a Nitrogen Atom (Specific Example Group G2A1):

a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

### Unsubstituted Heterocyclic Group Containing an Oxygen Atom (Specific Example Group G2A2):

a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

### Unsubstituted Heterocyclic Group Containing a Sulfur Atom (Specific Example Group G2A3):

a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).
   Monovalent Heterocyclic Group Derived by Removing One Hydrogen Atom from the Ring Structures Represented by Each of the Following General Formulas (TEMP-16) to (TEMP-33) (Specific Example Group G2A4):

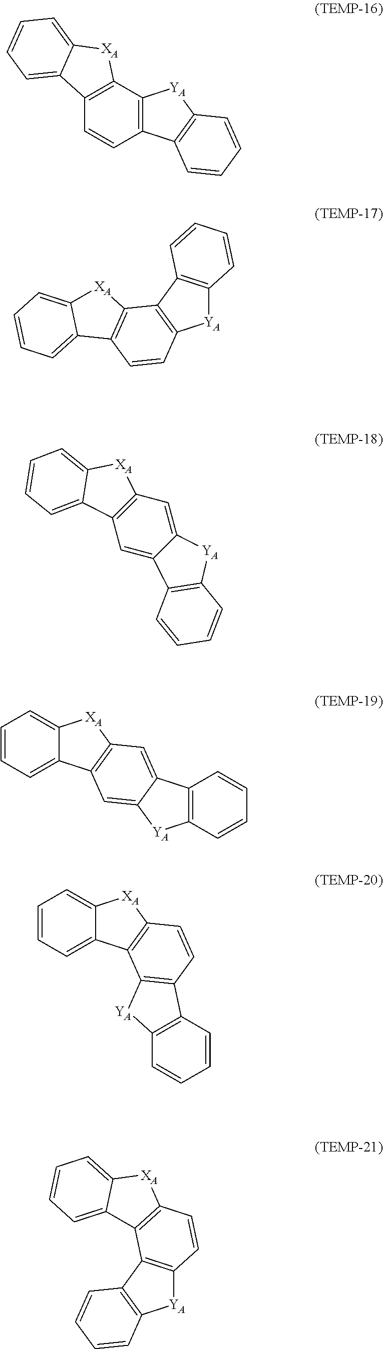

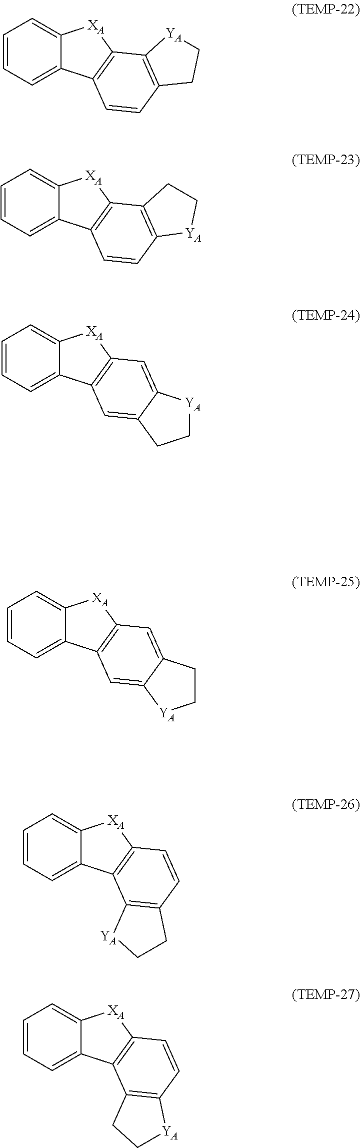

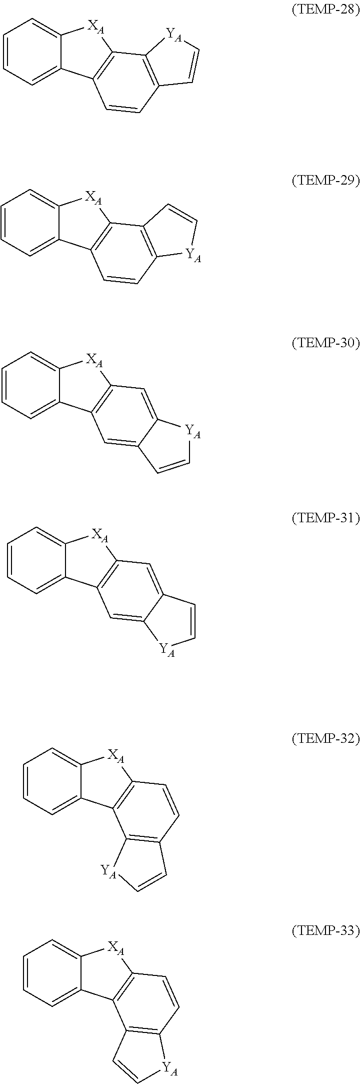

In the general formulas (TEMP-16) to (TEMP-33), X_{A} and Y_{A} are independently an oxygen atom, a sulfur atom, NH, or CH₂. Provided that at least one of X_{A} and Y_{A} is an oxygen atom, a sulfur atom, or NH.

In the general formulas (TEMP-16) to (TEMP-33), when at least one of X_{A} and Y_{A} is NH or CH₂, the monovalent heterocyclic group derived from the ring structures represented by each of the general formulas (TEMP-16) to (TEMP-33) includes a monovalent group derived by removing one hydrogen atom from these NH or CH₂.

### Substituted Heterocyclic Group Containing a Nitrogen Atom (Specific Example Group G2B1):

a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenylyltriazinyl group,
a diphenyltriazinyl group,
a phenylquinazolinyl group, and
a biphenylylquinazolinyl group.

### Substituted Heterocyclic Group Containing an Oxygen Atom (Specific Example Group G2B2):

a phenyldibenzofuranyl group,
a methyldibenzofuranyl group,
a t-butyldibenzofuranyl group, and
a monovalent residue of spiro[9H-xanthene-9,9′-[9H]fluorene].

### Substituted Heterocyclic Group Containing a Sulfur Atom (Specific Example Group G2B3):

a phenyldibenzothiophenyl group,
a methyldibenzothiophenyl group,
a t-butyldibenzothiophenyl group, and
a monovalent residue of spiro[9H-thioxanthene-9,9′-[9H]fluorene].
   Group in which One or More Hydrogen Atoms of the Monovalent Heterocyclic Group Derived from the Ring Structures Represented by Each of the Following General Formulas (TEMP-16) to (TEMP-33) are Substituted by a Substituent (Specific Example Group G2B4):

The “one or more hydrogen atoms of the monovalent heterocyclic group” means one or more hydrogen atoms selected from hydrogen atoms bonded with ring carbon atoms of the monovalent heterocyclic group, a hydrogen atom bonded with a nitrogen atom when at least one of X_{A} and Y_{A} is NH, and hydrogen atoms of a methylene group when one of X_{A} and Y_{A} is CH₂.

### “Substituted or Unsubstituted Alkyl Group”

Specific examples of the “substituted or unsubstituted alkyl group” (specific example group G3) described in this specification include the following unsubstituted alkyl groups (specific example group G3A) and the following substituted alkyl groups (specific example group G3B). (Here, the unsubstituted alkyl group refers to the case where the “substituted or unsubstituted alkyl group” is an “alkyl group unsubstituted by a substituent”, and the substituted alkyl group refers to the case where the “substituted or unsubstituted alkyl group“is an” alkyl group substituted by a substituent”.). In this specification, in the case where simply referred as an “alkyl group” includes both the “unsubstituted alkyl group” and the “substituted alkyl group.”

The “substituted alkyl group” means a group in which one or more hydrogen atoms in the “unsubstituted alkyl group” are substituted by a substituent. Specific examples of the “substituted alkyl group” include groups in which one or more hydrogen atoms in the following “unsubstituted alkyl group” (specific example group G3A) are substituted by a substituent, the following substituted alkyl group (specific example group G3B), and the like. In this specification, the alkyl group in the “unsubstituted alkyl group” means a linear alkyl group. Thus, the “unsubstituted alkyl group” includes a straight-chain “unsubstituted alkyl group” and a branched-chain “unsubstituted alkyl group”. It should be noted that the examples of the “unsubstituted alkyl group” and the examples of the “substituted alkyl group” enumerated in this specification are mere examples, and the “substituted alkyl group” described in this specification includes a group in which hydrogen atom of the alkyl group itself in the “substituted alkyl group” of the specific example group G3B is further substituted by a substituent, and a group in which hydrogen atom of a substituent in the “substituted alkyl group” of the specific example group G3B is further substituted by a substituent.

### Unsubstituted Alkyl Group (Specific Example Group G3A):

a methyl group,
an ethyl group,
a n-propyl group,
an isopropyl group,
a n-butyl group,
an isobutyl group,
a s-butyl group, and
a t-butyl group.

### Substituted Alkyl Group (Specific Example Group G3B):

a heptafluoropropyl group (including isomers),
a pentafluoroethyl group,
a 2,2,2-trifluoroethyl group, and
a trifluoromethyl group.

### “Substituted or Unsubstituted Alkenyl Group”

Specific examples of the “substituted or unsubstituted alkenyl group” described in this specification (specific example group G4) include the following unsubstituted alkenyl group (specific example group G4A), the following substituted alkenyl group (specific example group G4B), and the like. (Here, the unsubstituted alkenyl group refers to the case where the “substituted or unsubstituted alkenyl group“is a” alkenyl group unsubstituted by a substituent”, and the “substituted alkenyl group” refers to the case where the “substituted or unsubstituted alkenyl group” is a “alkenyl group substituted by a substituent.”). In this specification, in the case where simply referred as an “alkenyl group” includes both the “unsubstituted alkenyl group” and the “substituted alkenyl group.”

The “substituted alkenyl group” means a group in which one or more hydrogen atoms in the “unsubstituted alkenyl group” are substituted by a substituent. Specific examples of the “substituted alkenyl group” include a group in which the following “unsubstituted alkenyl group” (specific example group G4A) has a substituent, the following substituted alkenyl group (specific example group G4B), and the like. It should be noted that the examples of the “unsubstituted alkenyl group” and the examples of the “substituted alkenyl group” enumerated in this specification are mere examples, and the “substituted alkenyl group” described in this specification includes a group in which a hydrogen atom of the alkenyl group itself in the “substituted alkenyl group” of the specific example group G4B is further substituted by a substituent, and a group in which a hydrogen atom of a substituent in the “substituted alkenyl group” of the specific example group G4B is further substituted by a substituent.

### Unsubstituted Alkenyl Group (Specific Example Group G4A):

a vinyl group,
an allyl group,
a 1-butenyl group,
a 2-butenyl group, and
a 3-butenyl group.

### Substituted Alkenyl Group (Specific Example Group G4B):

a 1,3-butanedienyl group,
a 1-methylvinyl group,
a 1-methylallyl group,
a 1,1-dimethylallyl group,
a 2-methylally group, and
a 1,2-dimethylallyl group.

### “Substituted or Unsubstituted Alkynyl Group”

Specific examples of the “substituted or unsubstituted alkynyl group” described in this specification (specific example group G5) include the following unsubstituted alkynyl group (specific example group G5A) and the like. (Here, the unsubstituted alkynyl group refers to the case where the “substituted or unsubstituted alkynyl group” is an “alkynyl group unsubstituted by a substituent”.). In this specification, in the case where simply referred as an “alkynyl group” includes both the “unsubstituted alkynyl group” and the “substituted alkynyl group.”

The “substituted alkynyl group” means a group in which one or more hydrogen atoms in the “unsubstituted alkynyl group” are substituted by a substituent. Specific examples of the “substituted alkynyl group” include a group in which one or more hydrogen atoms in the following “unsubstituted alkynyl group” (specific example group G5A) are substituted by a substituent, and the like.

### Unsubstituted Alkynyl Group (Specific Example Group G5A):

an ethynyl group.

### “Substituted or Unsubstituted Cycloalkyl Group”

Specific examples of the “substituted or unsubstituted cycloalkyl group” described in this specification (specific example group G6) include the following unsubstituted cycloalkyl group (specific example group G6A), the following substituted cycloalkyl group (specific example group G6B), and the like. (Here, the unsubstituted cycloalkyl group refers to the case where the “substituted or unsubstituted cycloalkyl group“is a” cycloalkyl group unsubstituted by a substituent”, and the substituted cycloalkyl group refers to the case where the “substituted or unsubstituted cycloalkyl group” is a “cycloalkyl group substituted by a substituent”.). In this specification, in the case where simply referred as a “cycloalkyl group” includes both the “unsubstituted cycloalkyl group” and the “substituted cycloalkyl group.”

The “substituted cycloalkyl group” means a group in which one or more hydrogen atoms in the “unsubstituted cycloalkyl group” are substituted by a substituent. Specific examples of the “substituted cycloalkyl group” include a group in which one or more hydrogen atoms in the following “unsubstituted cycloalkyl group” (specific example group G6A) are substituted by a substituent, and examples of the following substituted cycloalkyl group (specific example group G6B), and the like. It should be noted that the examples of the “unsubstituted cycloalkyl group” and the examples of the “substituted cycloalkyl group” enumerated in this specification are mere examples, and the “substituted cycloalkyl group” in this specification includes a group in which one or more hydrogen atoms bonded with the carbon atom of the cycloalkyl group itself in the “substituted cycloalkyl group” of the specific example group G6B are substituted by a substituent, and a group in which a hydrogen atom of a substituent in the “substituted cycloalkyl group” of specific example group G6B is further substituted by a substituent.

### Unsubstituted Cycloalkyl Group (Specific Example Group G6A):

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

### Substituted Cycloalkyl Group (Specific Example Group G6B):

a 4-methylcyclohexyl group.
   “Group Represented by —Si(R₉₀₁)(R₉₀₂)(R₉₀₃)”

Specific examples of the group represented by —Si(R₉₀₁)(R₉₀₂)(R₉₀₃) described in this specification (specific example group G7) include:
Si(G1)(G1)(G1),
Si(G1)(G2)(G2),
Si(G1)(G1)(G2),
Si(G2)(G2)(G2),
Si(G3)(G3)(G3), and
Si(G6)(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —Si(G1)(G1)(G1) are the same or different.

Plural G2's in —Si(G1)(G2)(G2) are the same or different.

Plural G1's in —Si(G1)(G1)(G2) are the same or different.

Plural G2's in —Si(G2)(G2)(G2) are be the same or different.

Plural G3's in —Si(G3)(G3)(G3) are the same or different.

Plural G6's in —Si(G6)(G6)(G6) are be the same or different.

### “Group Represented by —O—(R₉₀₄)”

Specific examples of the group represented by —O—(R₉₀₄) in this specification (specific example group G8) include:
—O(G1),
—O(G2),
—O(G3), and
—O(G6).
G1 is the “substituted or unsubstituted aryl group” described in the specific example group G1.
G2 is the “substituted or unsubstituted heterocyclic group” described in the specific example group G2.
G3 is the “substituted or unsubstituted alkyl group” described in the specific example group G3.
G6 is the “substituted or unsubstituted cycloalkyl group” described in the specific example group G6.

### “Group Represented by —S—(R₉₀₅)”

Specific examples of the group represented by —S—(R₉₀₅) in this specification (specific example group G9) include:
—S(G1),
—S(G2),
—S(G3), and
—S(G6).
G1 is the “substituted or unsubstituted aryl group” described in the specific example group G1.
G2 is the “substituted or unsubstituted heterocyclic group” described in the specific example group G2.
G3 is the “substituted or unsubstituted alkyl group” described in the specific example group G3.
G6 is the “substituted or unsubstituted cycloalkyl group” described in the specific example group G6.
   “Group Represented by —N(R₉₀₆)(R₉₀₇)”

Specific examples of the group represented by —N(R₉₀₆)(R₉₀₇) in this specification (specific example group G10) include:
—N(G1)(G1),
—N(G2)(G2),
—N(G1)(G2),
—N(G3)(G3), and
—N(G6)(G6).
G1 is the “substituted or unsubstituted aryl group” described in the specific example group G1.
G2 is the “substituted or unsubstituted heterocyclic group” described in the specific example group G2.
G3 is the “substituted or unsubstituted alkyl group” described in the specific example group G3.
G6 is the “substituted or unsubstituted cycloalkyl group” described in the specific example group G6.
Plural G1's in —N(G1)(G1) are the same or different.
Plural G2's in —N(G2)(G2) are the same or different.
Plural G3's in —N(G3)(G3) are the same or different.
Plural G6's in —N(G6)(G6) are the same or different.

### “Halogen Atom”

Specific examples of the “halogen atom” described in this specification (specific example group G11) include a fluorine atom, a chlorine atom, a bromine atom, an iodine atom, and the like.

### “Substituted or Unsubstituted Fluoroalkyl Group”

The “substituted or unsubstituted fluoroalkyl group” described in this specification is a group in which at least one hydrogen atom bonded with a carbon atom constituting the alkyl group in the “substituted or unsubstituted alkyl group” is substituted by a fluorine atom, and includes a group in which all hydrogen atoms bonded with a carbon atom constituting the alkyl group in the “substituted or unsubstituted alkyl group” are substituted by a fluorine atom (a perfluoro group). The number of carbon atoms of the “unsubstituted fluoroalkyl group” is 1 to 50, preferably 1 to 30, more preferably 1 to 18, unless otherwise specified in this specification. The “substituted fluoroalkyl group” means a group in which one or more hydrogen atoms of the “fluoroalkyl group” are substituted by a substituent. The “substituted fluoroalkyl group” described in this specification also includes a group in which one or more hydrogen atoms bonded with a carbon atom of the alkyl chains in the “substituted fluoroalkyl group” are further substituted by a substituent, and a group in which one or more hydrogen atom of a substituent in the “substituted fluoroalkyl group” are further substituted by a substituent. Specific examples of the “unsubstituted fluoroalkyl group” include a group in which one or more hydrogen atoms in the “alkyl group” (specific group G3) are substituted by a fluorine atom, and the like.

### “Substituted or Unsubstituted Haloalkyl Group”

The “substituted or unsubstituted haloalkyl group” described in this specification is a group in which at least one hydrogen atom bonded with a carbon atom constituting the alkyl group in the “substituted or unsubstituted alkyl group” is substituted by a halogen atom, and also includes a group in which all hydrogen atoms bonded with a carbon atom constituting the alkyl group in the “substituted or unsubstituted alkyl group” are substituted by a halogen atom. The number of carbon atoms of the “unsubstituted haloalkyl group” is 1 to 50, preferably 1 to 30, more preferably 1 to 18, unless otherwise specified in this specification. The “substituted haloalkyl group” means a group in which one or more hydrogen atoms of the “haloalkyl group” are substituted by a substituent. The “substituted haloalkyl group” described in this specification also includes a group in which one or more hydrogen atoms bonded with a carbon atom of the alkyl chain in the “substituted haloalkyl group” are further substituted by a substituent, and a group in which one or more hydrogen atoms of a substituent in the “substituted haloalkyl group” are further substituted by a substituent. Specific examples of the “unsubstituted haloalkyl group” include a group in which one or more hydrogen atoms in the “alkyl group” (specific example group G3) are substituted by a halogen atom, and the like. A haloalkyl group is sometimes referred to as an alkyl halide group.

### “Substituted or Unsubstituted Alkoxy Group”

Specific examples of the “substituted or unsubstituted alkoxy group” described in this specification include a group represented by —O(G3), wherein G3 is the “substituted or unsubstituted alkyl group” described in the specific example group G3. The number of carbon atoms of the “unsubstituted alkoxy group” is 1 to 50, preferably 1 to 30, more preferably 1 to 18, unless otherwise specified in this specification.

### “Substituted or Unsubstituted Alkylthio Group”

Specific examples of the “substituted or unsubstituted alkylthio group” described in this specification include a group represented by —S(G3), wherein G3 is the “substituted or unsubstituted alkyl group” described in the specific example group G3. The number of carbon atoms of the “unsubstituted alkylthio group” is 1 to 50, preferably 1 to 30, more preferably 1 to 18, unless otherwise specified in this specification.

“Substituted or Unsubstituted Aryloxy Group” Specific examples of the “substituted or unsubstituted aryloxy group” described in this specification include a group represented by —O(G1), wherein G1 is the “substituted or unsubstituted aryl group” described in the specific example group G1. The number of ring carbon atoms of the “unsubstituted aryloxy group” is 6 to 50, preferably 6 to 30, more preferably 6 to 18, unless otherwise specified in this specification.

### “Substituted or Unsubstituted Arylthio Group”

Specific examples of the “substituted or unsubstituted arylthio group” described in this specification include a group represented by —S(G1), wherein G1 is a “substituted or unsubstituted aryl group” described in the specific example group G1. The number of ring carbon atoms of the “unsubstituted arylthio group” is 6 to 50, preferably 6 to 30, more preferably 6 to 18, unless otherwise specified in this specification.

### “Substituted or Unsubstituted Trialkylsilyl Group”

Specific examples of the “trialkylsilyl group” described in this specification include a group represented by —Si(G3)(G3)(G3), where G3 is the “substituted or unsubstituted alkyl group” described in the specific example group G3. Plural G3's in —Si(G3)(G3)(G3) are the same or different. The number of carbon atoms in each alkyl group of the “trialkylsilyl group” is 1 to 50, preferably 1 to 20, more preferably 1 to 6, unless otherwise specified in this specification.

### “Substituted or Unsubstituted Aralkyl Group”

Specific examples of the “substituted or unsubstituted aralkyl group” described in this specification is a group represented by -(G3)-(G1), wherein G3 is the “substituted or unsubstituted alkyl group” described in the specific example group G3, and G1 is the “substituted or unsubstituted aryl group” described in the specific example group G1. Therefore, the “aralkyl group” is a group in which a hydrogen atom of the “alkyl group” is substituted by an “aryl group” as a substituent, and is one form of the “substituted alkyl group.” The “unsubstituted aralkyl group” is the “unsubstituted alkyl group” substituted by the “unsubstituted aryl group”, and the number of carbon atoms of the “unsubstituted aralkyl group” is 7 to 50, preferably 7 to 30, more preferably 7 to 18, unless otherwise specified in this specification.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, and the like.

Unless otherwise specified in this specification, examples of the substituted or unsubstituted aryl group described in this specification preferably include a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, 9,9-dimethylfluorenyl group, 9,9-diphenylfluorenyl group, and the like.

Unless otherwise specified in this specification, examples of the substituted or unsubstituted heterocyclic groups described in this specification preferably include a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like.

In this specification, the carbazolyl group is specifically each of the following groups, unless otherwise specified in this specification.

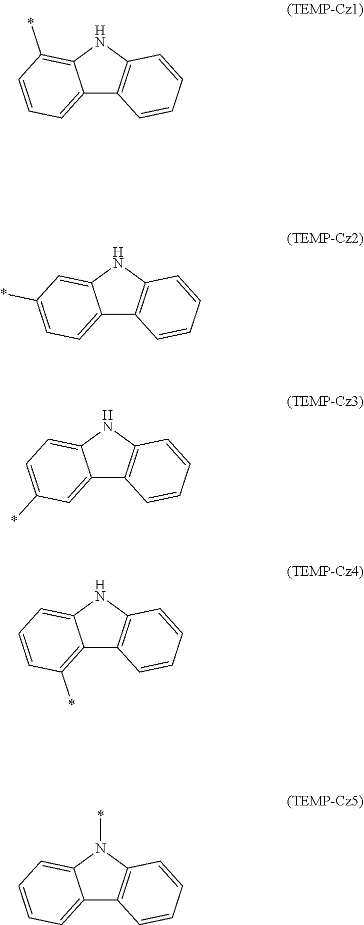 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In this specification, the (9-phenyl)carbazolyl group is specifically any of the following groups, unless otherwise specified in this specification.

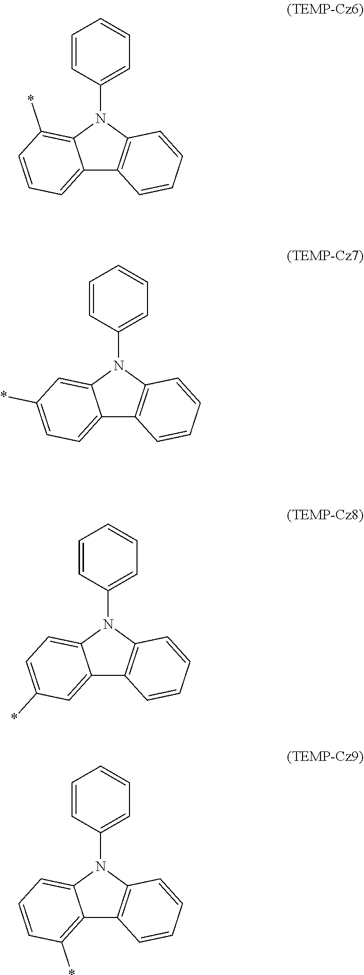 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the general formulas (TEMP-Cz1) to (TEMP-Cz9), * represents a bonding site.

In this specification, the dibenzofuranyl group and the dibenzothiophenyl group are specifically any of the following groups, unless otherwise specified in this specification.

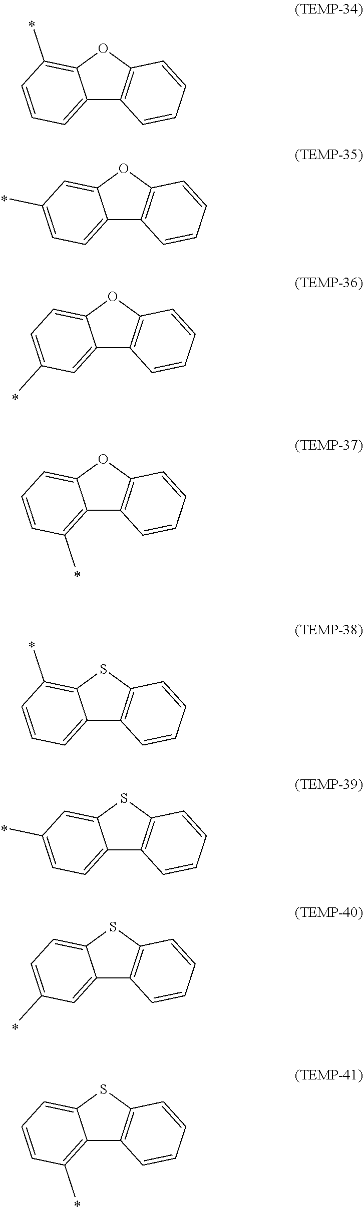

In the general formulas (TEMP-34) to (TEMP-41), * represents a bonding site.

The substituted or unsubstituted alkyl group described in this specification is preferably a methyl group, an ethyl group, a propyl group, an isopropyl group, a n-butyl group, an isobutyl group, a t-butyl group, or the like, unless otherwise specified in this specification.

### “Substituted or Unsubstituted Arylene Group”

The “substituted or unsubstituted arylene group” described in this specification is a divalent group derived by removing one hydrogen atom on the aryl ring of the “substituted or unsubstituted aryl group”, unless otherwise specified. Specific examples of the “substituted or unsubstituted arylene group” (specific example group G12) include a divalent group derived by removing one hydrogen atom on the aryl ring of the “substituted or unsubstituted aryl group” described in the specific example group G1, and the like.

### “Substituted or Unsubstituted Divalent Heterocyclic Group”

The “substituted or unsubstituted divalent heterocyclic group” described in this specification is a divalent group derived by removing one hydrogen atom on the heterocycle of the “substituted or unsubstituted heterocyclic group”, unless otherwise specified. Specific examples of the “substituted or unsubstituted divalent heterocyclic group” (specific example group G13) include a divalent group derived by removing one hydrogen atom on the heterocycle of the “substituted or unsubstituted heterocyclic group” described in the specific example group G2, and the like.

### “Substituted or Unsubstituted Alkylene Group”

The “substituted or unsubstituted alkylene group” described in this specification is a divalent group derived by removing one hydrogen atom on the alkyl chain of the “substituted or unsubstituted alkyl group”, unless otherwise specified. Specific examples of the “substituted or unsubstituted alkylene group” (specific example group G14) include a divalent group derived by removing one hydrogen atom on the alkyl chain of the “substituted or unsubstituted alkyl group” described in the specific example group G3, and the like.

The substituted or unsubstituted arylene group described in this specification is preferably any group of the following general formulas (TEMP-42) to (TEMP-68), unless otherwise specified in this specification.

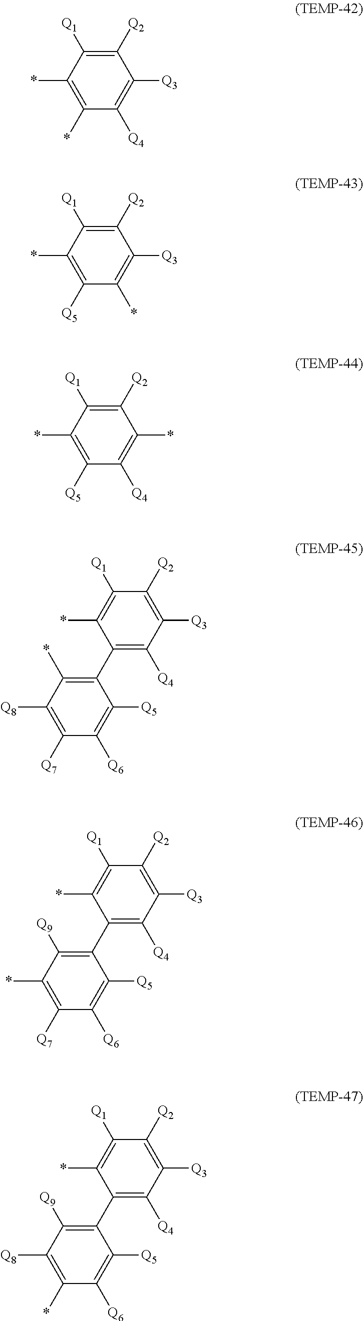

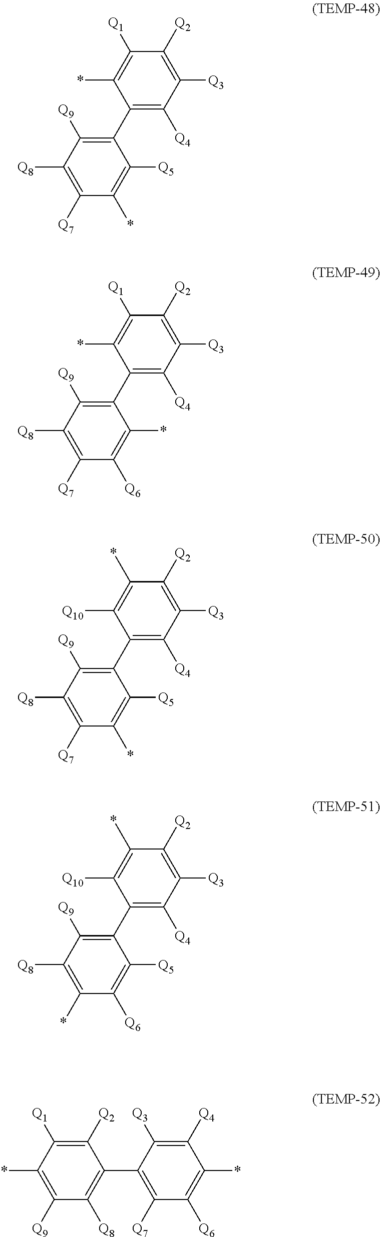

In the general formulas (TEMP-42) to (TEMP-52), Q₁ to Q₁₀ are independently a hydrogen atom or a substituent.

In the general formulas (TEMP-42) to (TEMP-52), * represents a bonding site.

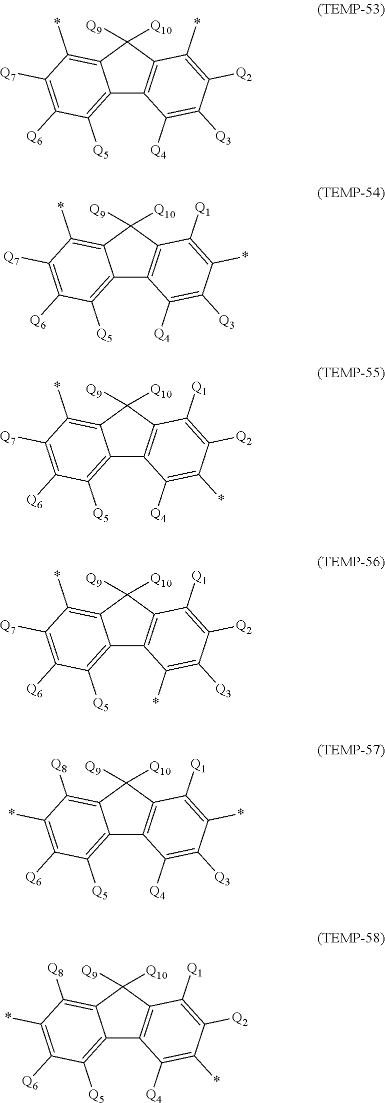

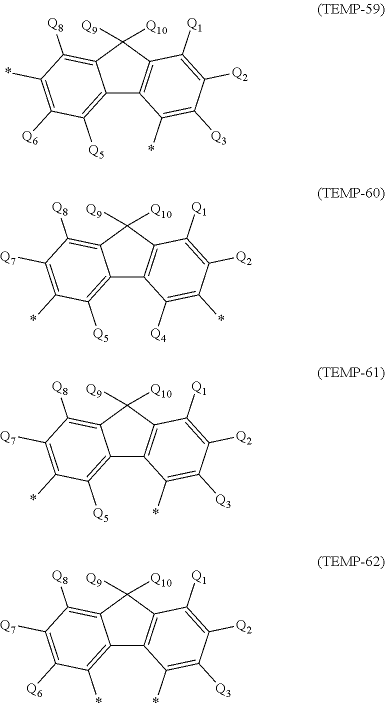

In the general formulas (TEMP-53) to (TEMP-62), Q₁ to Q₁₀ are independently a hydrogen atom or a substituent.

Q₉ and Q₁₀ may be bonded with each other via a single bond to form a ring.

In the general formulas (TEMP-53) to (TEMP-62), * represents a bonding site.

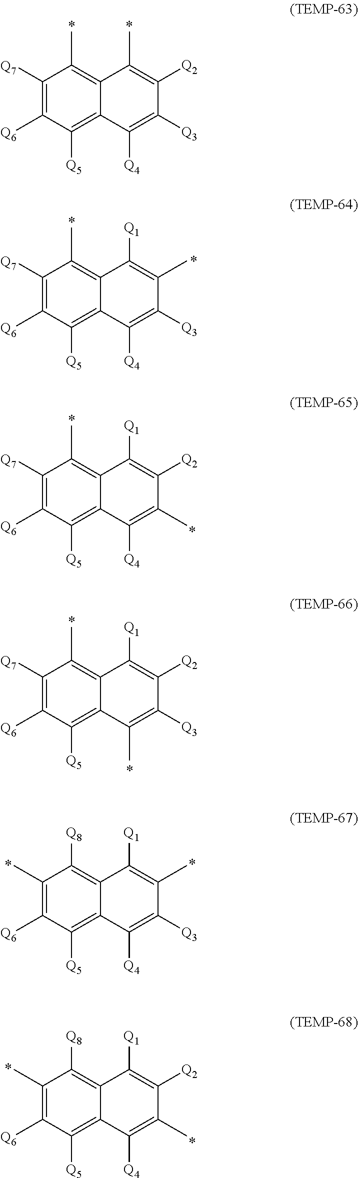

In the general formulas (TEMP-63) to (TEMP-68), Q₁ to Q₈ are independently a hydrogen atom or a substituent.

In the general formulas (TEMP-63) to (TEMP-68), * represents a bonding site.

The substituted or unsubstituted divalent heterocyclic group described in this specification is preferably any group of the following general formulas (TEMP-69) to (TEMP-102), unless otherwise specified in this specification.

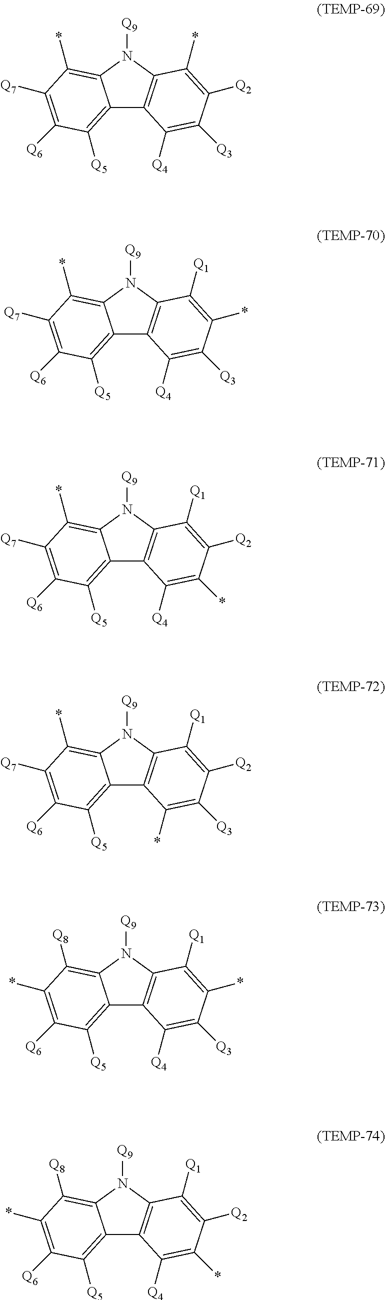

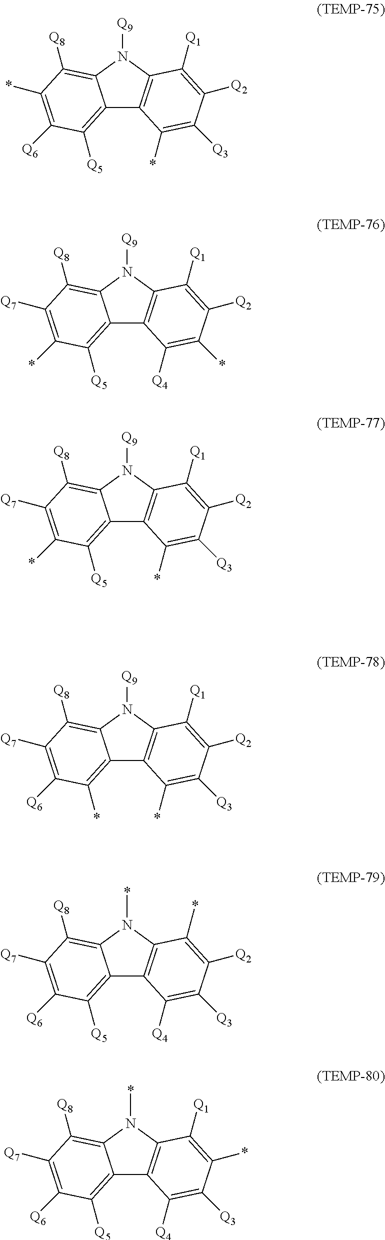

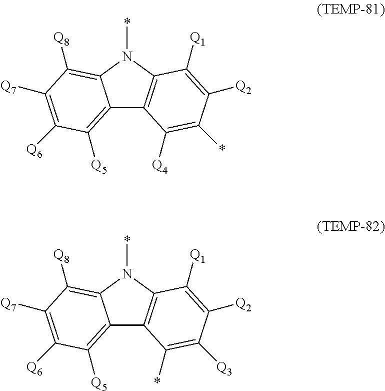

In the general formulas (TEMP-69) to (TEMP-82), Q₁ to Q₉ are independently a hydrogen atom or a substituent.

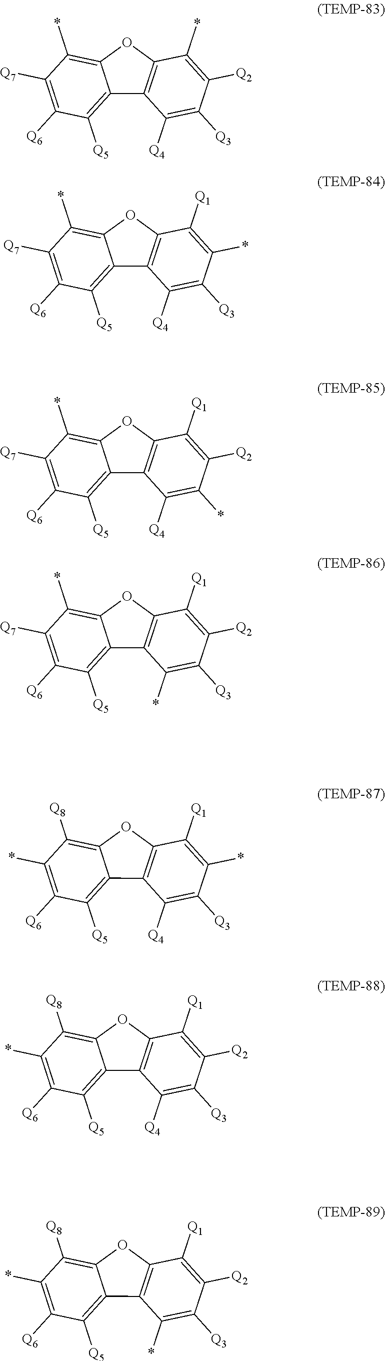

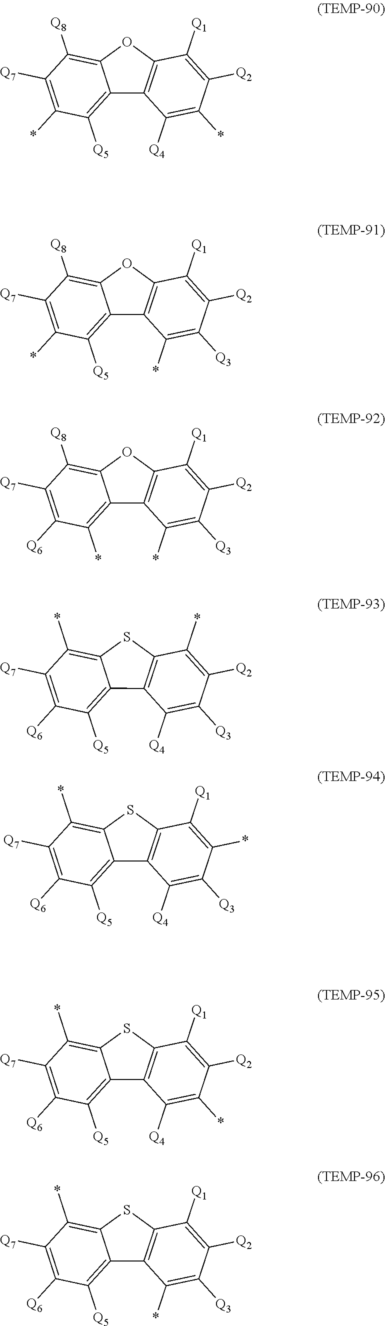

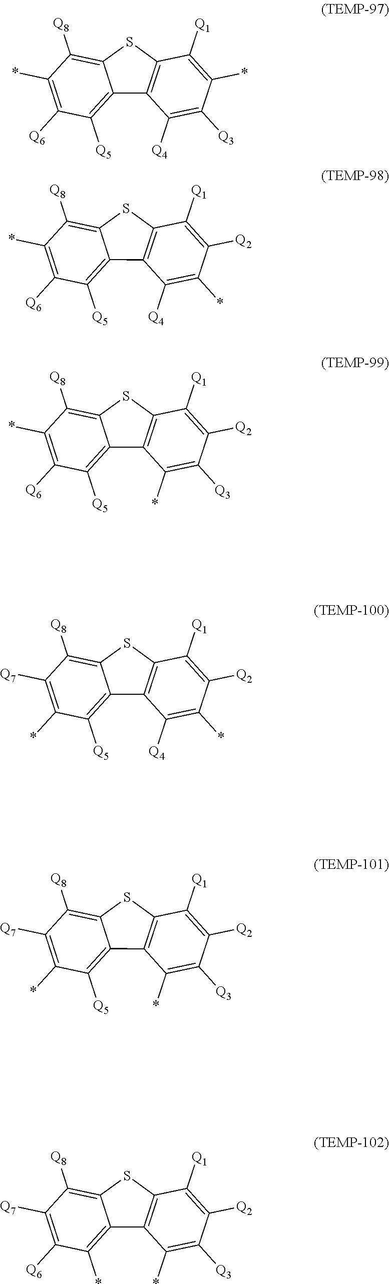

In the general formulas (TEMP-83) to (TEMP-102), Q₁ to Q₈ are independently a hydrogen atom or a substituent.

The above is the explanation of the “Substituent described in this specification.”

“The Case where Bonded with Each Other to Form a Ring”

In this specification, the case where “one or more sets of adjacent two or more form a substituted or unsubstituted monocycle by bonding with each other, form a substituted or unsubstituted fused ring by bonding with each other, or do not bond with each other” means the case where “one or more sets of adjacent two or more form a substituted or unsubstituted monocycle by bonding with each other”; the case where “one or more sets of adjacent two or more form a substituted or unsubstituted fused ring by bonding with each other”; and the case where “one or more sets of adjacent two or more do not bond with each other.”

The case where “one or more sets of adjacent two or more form a substituted or unsubstituted monocycle by bonding with each other” and the case where “one or more sets of adjacent two or more form a substituted or unsubstituted fused ring by bonding with each other” in this specification (these cases may be collectively referred to as “the case where forming a ring by bonding with each other”) will be described below. The case of an anthracene compound represented by the following general formula (TEMP-103) in which the mother skeleton is an anthracene ring will be described as an example.

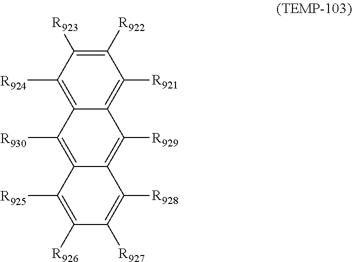

For example, in the case where “one or more sets of adjacent two or more among R₉₂₁ to R₉₃₀ form a ring by bonding with each other”, the one set of adjacent two includes a pair of R₉₂₁ and R₉₂₂, a pair of R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₆, a pair of R₉₂₆ and R₉₂₇, a pair of R₉₂₇ and R₉₂₈, a pair of R₉₂₈ and R₉₂₉, and a pair of R₉₂₉ and R₉₂₁.

The “one or more sets” means that two or more sets of the adjacent two or more sets may form a ring at the same time. For example, R₉₂₁ and R₉₂₂ form a ring Q_{A} by bonding with each other, and at the same, time R₉₂₅ and R₉₂₆ form a ring Q_{B} by bonding with each other, the anthracene compound represented by the general formula (TEMP-103) is represented by the following general formula (TEMP-104).

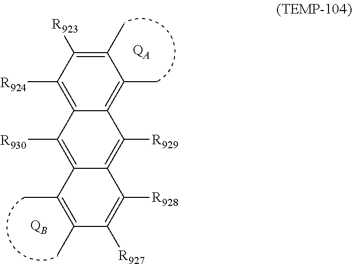

The case where the “set of adjacent two or more” form a ring includes not only the case where the set (pair) of adjacent “two” is bonded with as in the above-mentioned examples, but also the case where the set of adjacent “three or more” are bonded with each other. For example, it means the case where R₉₂₁ and R₉₂₂ form a ring Q_{A} by bonding with each other, and R₉₂₂ and R₉₂₃ form a ring Qc by bonding with each other, and adjacent three (R₉₂₁, R₉₂₂ and R₉₂₃) form rings by bonding with each other and together fused to the anthracene mother skeleton. In this case, the anthracene compound represented by the general formula (TEMP-103) is represented by the following general formula (TEMP-105). In the following general formula (TEMP-105), the ring Q_{A} and the ring Qc share R₉₂₂.

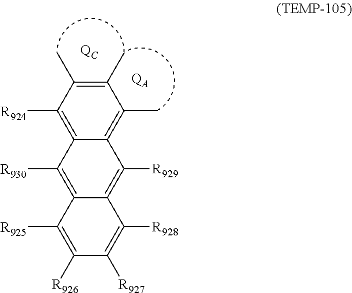

The “monocycle” or “fused ring” formed may be a saturated ring or an unsaturated ring, as a structure of the formed ring alone. Even when the “one pair of adjacent two” forms a “monocycle” or a “fused ring”, the “monocycle” or the “fused ring” may form a saturated ring or an unsaturated ring. For example, the ring Q_{A} and the ring Q_{B} formed in the general formula (TEMP-104) are independently a “monocycle” or a “fused ring.” The ring Q_{A} and the ring Qc formed in the general formula (TEMP-105) are “fused ring.” The ring Q_{A} and ring Qc of the general formula (TEMP-105) are fused ring by fusing the ring Q_{A} and the ring Qc together. When the ring Q_{A} of the general formula (TMEP-104) is a benzene ring, the ring Q_{A} is a monocycle. When the ring Q_{A} of the general formula (TMEP-104) is a naphthalene ring, the ring Q_{A} is a fused ring.

The “unsaturated ring” includes, in addition to an aromatic hydrocarbon ring and an aromatic heterocycle, an aliphatic hydrocarbon ring with an unsaturated bond, i.e., double and/or triple bonds in the ring structure (e.g., cyclohexene, cyclohexadiene, etc.), and a non-aromatic heterocycle with an unsaturated bond (e.g., dihydropyran, imidazoline, pyrazoline, quinolizine, indoline, isoindoline, etc.). The “saturated ring” includes an aliphatic hydrocarbon ring without an unsaturated bond and a non-aromatic heterocycle without ab unsaturated bond.

Specific examples of the aromatic hydrocarbon ring include a structure in which the group listed as a specific example in the specific example group G1 is terminated by a hydrogen atom.

Specific examples of the aromatic heterocycle include a structure in which the aromatic heterocyclic group listed as a specific example in the example group G2 is terminated by a hydrogen atom.

Specific examples of the aliphatic hydrocarbon ring include a structure in which the group listed as a specific example in the specific example group G6 is terminated by a hydrogen atom.

The term “to form a ring” means forming a ring only with plural atoms of the mother skeleton, or with plural atoms of the mother skeleton and one or more arbitrary atoms in addition. For example, the ring Q_{A} shown in the general formula (TEMP-104), which is formed by bonding R₉₂₁ and R₉₂₂ with each other, is a ring formed from the carbon atom of the anthracene skeleton with which R₉₂₁ is bonded, the carbon atom of the anthracene skeleton with which R₉₂₂ is bonded, and one or more arbitrary atoms. For example, in the case where the ring Q_{A} is formed with R₉₂₁ and R₉₂₂, when a monocyclic unsaturated ring is formed with the carbon atom of the anthracene skeleton with which R₉₂₁ is bonded, the carbon atom of the anthracene skeleton with which R₉₂₂ is bonded, and four carbon atoms, the ring formed with R₉₂₁ and R₉₂₂ is a benzene ring.

Here, the “arbitrary atom” is preferably at least one atom selected from the group consisting of a carbon atom, a nitrogen atom, an oxygen atom, and a sulfur atom, unless otherwise specified in this specification. In the arbitrary atom (for example, a carbon atom or a nitrogen atom), a bond which does not form a ring may be terminated with a hydrogen atom or the like, or may be substituted with “arbitrary substituent” described below. When an arbitrary atom other than a carbon atom is contained, the ring formed is a heterocycle.

The number of “one or more arbitrary atom(s)” constituting a monocycle or a fused ring is preferably 2 or more and 15 or less, more preferably 3 or more and 12 or less, and still more preferably 3 or more and 5 or less, unless otherwise specified in this specification.

The “monocycle” is preferable among the “monocycle” and the “fused ring”, unless otherwise specified in this specification.

The “unsaturated ring” is preferable among the “saturated ring” and the “unsaturated ring”, unless otherwise specified in this specification.

Unless otherwise specified in this specification, the “monocycle” is preferably a benzene ring.

Unless otherwise specified in this specification, the “unsaturated ring” is preferably a benzene ring.

Unless otherwise specified in this specification, when “one or more sets of adjacent two or more” are “bonded with each other to form a substituted or unsubstituted monocycle” or “bonded with each other to form a substituted or unsubstituted fused ring”, this specification, one or more sets of adjacent two or more are preferably bonded with each other to form a substituted or unsubstituted “unsaturated ring” from plural atoms of the mother skeleton and one or more and 15 or less atoms which is at least one kind selected from a carbon atom, a nitrogen atom, an oxygen atom, and a sulfur atom.

The substituent in the case where the above-mentioned “monocycle” or “fused ring” has a substituent is, for example, an “arbitrary substituent” described below. Specific examples of the substituent which the above-mentioned “monocycle” or “fused ring” has include the substituent described above in the “Substituent described in this specification” section.

The substituent in the case where the above-mentioned “saturated ring” or “unsaturated ring” has a substituent is, for example, an “arbitrary substituent” described below. Specific examples of the substituent which the above-mentioned “monocycle” or “fused ring” has include the substituent described above in the “Substituent described in this specification” section.

The foregoing describes the case where “one or more sets of adjacent two or more form a substituted or unsubstituted monocycle by bonding with each other” and the case where “one or more sets of adjacent two or more form a substituted or unsubstituted fused ring by bonding with each other” (the case where “forming a ring by bonding with each other”).

### Substituent in the Case of “Substituted or Unsubstituted”

In one embodiment in this specification, the substituent (in this specification, sometimes referred to as an “arbitrary substituent”) in the case of “substituted or unsubstituted” is, for example, a group selected from the group consisting of:
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, and
an unsubstituted heterocyclic group including 5 to 50 ring atoms,
wherein, R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted heterocyclic group including 5 to 50 ring atoms.

When two or more R₉₀₁'s are present, the two or more R₉₀₁'s may be the same or different.

When two or more R₉₀₂'s are present, the two or more R₉₀₂'s may be the same or different.

When two or more R₉₀₃'s are present, the two or more R₉₀₃'s may be the same or different.

When two or more R₉₀₄'s are present, the two or more R₉₀₄'s may be the same or different.

When two or more R₉₀₅'s are present, the two or more R₉₀₅'s may be the same or different.

When two or more R₉₀₆'s are present, the two or more R₉₀₆'s may be the same or different.

When two or more R₉₀₇'s are present, the two or more R₉₀₇'s may be the same or different.

In one embodiment, the substituent in the case of “substituted or unsubstituted” is a group selected from the group consisting of:
an alkyl group including 1 to 50 carbon atoms,
an aryl group including 6 to 50 ring carbon atoms, and
a heterocyclic group including 5 to 50 ring atoms.

In one embodiment, the substituent in the case of “substituted or unsubstituted” is a group selected from the group consisting of:
an alkyl group including 1 to 18 carbon atoms,
an aryl group including 6 to 18 ring carbon atoms, and
a heterocyclic group including 5 to 18 ring atoms.

Specific examples of each of the arbitrary substituents include specific examples of substituent described in the section “Substituent described in this specification” above.

Unless otherwise specified in this specification, adjacent arbitrary substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, more preferably form a benzene ring.

Unless otherwise specified in this specification, the arbitrary substituent may further have a substituent. The substituent which the arbitrary substituent further has is the same as that of the above-mentioned arbitrary substituent.

In this specification, the numerical range represented by “AA to BB” means the range including the numerical value AA described on the front side of “AA to BB” as the lower limit and the numerical value BB described on the rear side of “AA to BB” as the upper limit.

### [Novel Compound]

A compound according to one aspect of the invention is represented by the following formula (1):

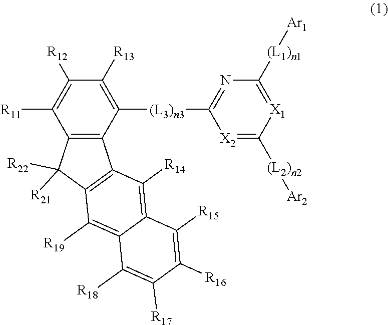

wherein in the formula (1),
X₁ and X₂ are independently N or CH, and any one of X₁ and X₂ is N,
Ar₁ and Ar₂ are independently
a substituted or unsubstituted aryl group including 6 to 50 ring carbon atoms, or
a substituted or unsubstituted monovalent heterocyclic group including 5 to 50 ring atoms,
L₁ to L₃ are independently
a single bond,
a substituted or unsubstituted arylene group including 6 to 50 ring carbon atoms, or
a substituted or unsubstituted divalent heterocyclic group including 5 to 50 ring atoms,
when n1 is an integer of 0 to 4 and n1 is 0, (L₁)ₙ₁ is a single bond,
when a plurality of L₁'s is present, the plurality of L₁'s is the same as or different from each other,
when n2 is an integer of 0 to 4 and n2 is 0, (L₂)ₙ₂ is a single bond,
when a plurality of L₂'s is present, the plurality of L₂'s is the same as or different from each other,
when n3 is an integer of 0 to 4 and n3 is 0, (L₃)ₙ₃ is a single bond,
when a plurality of L₃'s is present, the plurality of L₃'s is the same as or different from each other,
R₁₁ to R₁₉ are independently a hydrogen atom or a substituent R,
a set of adjacent two or more of R₁₁ to R₁₉ does not bond with each other,
R₂₁ and R₂₂ form a substituted or unsubstituted saturated or unsaturated ring by bonding with each other or do not form a substituted or unsubstituted saturated or unsaturated ring by bonding with each other,
R₂₁ and R₂₂ which do not form a substituted or unsubstituted saturated or unsaturated ring are independently a hydrogen atom, or a substituted or unsubstituted alkyl group including 1 to 50 carbon atoms,
the substituent R is selected from the group consisting of,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms, and
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms,
when two or more of each of R₉₀₁ to R₉₀₇ are present, the two or more of each of R₉₀₁ to R₉₀₇ may be the same as or different from each other, and
when two or more substituent R's are present, the two or more substituent R's may be the same as or different from each other.

In the formula (1), any one of X₁ and X₂ is N and the other is CH. That is, when X₁ is N, X₂ is CH. When X₂ is N, X₁ is CH.

In the formula (1), when a plurality of L₁'s is present (in the case of n1 being 2 or more), Ar₁ is bonded to L₁ most distant from the nitrogen-containing six-membered ring containing X₁ and X₂. When a plurality of L₂'s is present (in the case of n2 being two or more), Ar₂ is also bonded to L₂ most distant from the nitrogen-containing six-membered ring containing X₁ and X₂. Similarly, when a plurality of L₃'s is present (in the case of n3 being 2 or more), the benzofluorene skeleton is bonded to L₃ most distant from the nitrogen-containing six-membered ring containing X₁ and X₂.

In one embodiment, the compound represented by the formula (1) is a compound represented by the following formula (11):

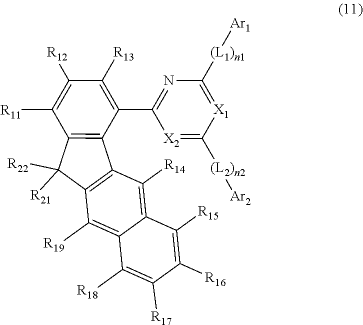

wherein in the formula (11),
X₁, X₂, Ar₁, Ar₂, L₁, L₂, n1, n2, R₁₁ to R₁₉, R₂₁, and R₂₂ are as defined in the formula (1).

In one embodiment, R₂₁ and R₂₂ in the formula (11) do not form a substituted or unsubstituted saturated or unsaturated ring.

In one embodiment, R₂₁ and R₂₂ in the formula (11) are substituted or unsubstituted alkyl groups including 1 to 5 carbon atoms, for example, methyl groups.

In one embodiment, X₁ in the formula (11) is N and X₂ is CH.

In one embodiment, X₁ in the formula (11) is CH and X₂ is N.

In one embodiment, the compound represented by the formula (11) is a compound represented by the following formula (111) or (112):

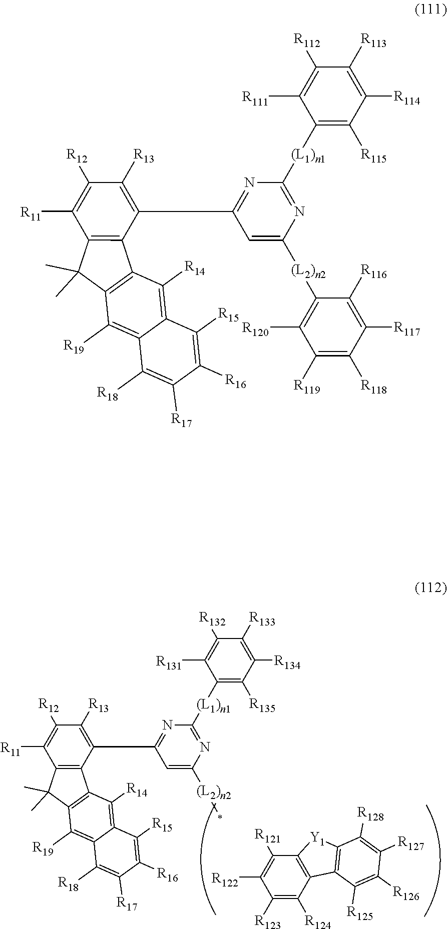

wherein in the formula (111),
L₁, L₂, n1, n2, and R₁₁ to R₁₉ are as defined in the formula (1), one or more sets of adjacent two or more of R₁₁₁ to R₁₁₅ form a substituted or unsubstituted saturated or unsaturated ring by bonding with each other or do not bond with each other,
R₁₁₁ to R₁₁₅ which do not form a substituted or unsubstituted saturated or unsaturated ring are independently a hydrogen atom or a substituent R,
one or more sets of adjacent two or more of R₁₁₆ to R₁₂₀ form a substituted or unsubstituted saturated or unsaturated ring by bonding with each other or do not bond with each other,
R₁₁₆ to R₁₂₀ which do not form a substituted or unsubstituted saturated or unsaturated ring are independently a hydrogen atom or a substituent R,
wherein in the formula (112),
L₁, L₂, n1, n2, and R₁₁ to R₁₉ are as defined in the formula (1),
Y₁ is N(R₁₂₉), C(R₁₃₀ₐ)(R_{130b}), O, or S,
one or more sets of adjacent two or more of R₁₂₁ to R₁₂₉ are form a substituted or unsubstituted saturated or unsaturated ring by bonding with each other or do not bond with each other,
(L₂)ₙ₂ bonds any one of bondable positions of a fused ring structure in parentheses,
R₁₂₁ to R₁₂₉ which do not bond with each other and do not bond with (L₂)ₙ₂ are independently a hydrogen atom or a substituent R,
R₁₃₀ₐ and R_{130b} form a substituted or unsubstituted saturated or unsaturated ring by bonding with each other or do not form a substituted or unsubstituted saturated or unsaturated ring by bonding with each other,
R₁₃₀ₐ and R_{130b} which do not form a substituted or unsubstituted saturated or unsaturated ring are independently a hydrogen atom, or a substituent R,
one or more sets of adjacent two or more of R₁₃₁ to R₁₃₅ form a substituted or unsubstituted saturated or unsaturated ring by bonding with each other or do not bond with each other,
R₁₃₁ to R₁₃₅ which do not form the substituted or unsubstituted saturated or unsaturated ring are independently a hydrogen atom or a substituent R, and
the substituent R is as defined in the formula (1).

In one embodiment, in the formulas (111) and (112), one or more sets of two or more of R₁₁₁ to R₁₁₅ do not bond with each other, one or more sets of adjacent two or more of R₁₁₆ to R₁₂₀ do not bond with each other, one or more sets of adjacent two or more of R₁₂₁ to R₁₂₉ do not bond with each other, and one or more sets of adjacent two or more of R₁₃₁ to R₁₃₅ do not bond with each other.

In one embodiment, R₁₂₃ in the formula (112) represents a bond with L₂.

When R₁₂₃ represents a bond with L₂, the compound represented by the formula (112) is the following structure:

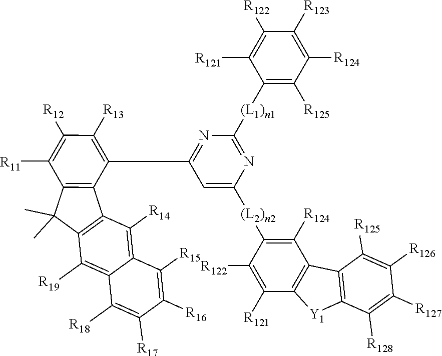

In one embodiment, Y₁ is O.

In one embodiment, the compound represented by the formula (1) is a compound represented by the following formula (21):

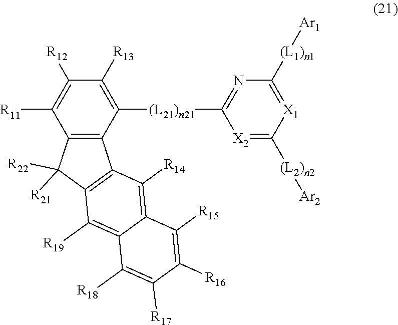

wherein in the formula (21),
X₁, X₂, Ar₁, Ar₂, L₁, L₂, n1, n2, R₁₁ to R₁₉, R₂₁, and R₂₂ are as defined in the formula (1),
L₂₁ is
a substituted or unsubstituted arylene group including 6 to 50 ring carbon atoms, or
a substituted or unsubstituted divalent heterocyclic group including 5 to 50 ring atoms,
n21 is an integer of 1 to 4, and
when a plurality of L₂₁'s is present, the plurality of L₂₁'s may be the same as or different from each other.

In one embodiment, R₂₁ and R₂₂ in the formula (21) do not form a substituted or unsubstituted saturated or unsaturated ring.

In one embodiment, R₂₁ and R₂₂ in the formula (21) is substituted or unsubstituted alkyl groups including 1 to 5 carbon atoms, for example, methyl groups.

In one embodiment, L₂₁ in the formula (21) is a substituted or unsubstituted arylene group including 6 to 50 ring carbon atoms.

In one embodiment, L₂₁ in the formula (21) is a substituted or unsubstituted phenylene group or a substituted or unsubstituted naphthylene group.

In one embodiment, X₁ in the formula (21) is N and X₂ is CH.

In one embodiment, X₁ in the formula (21) is CH and X₂ is N.

In one embodiment, the compound represented by the formula (21) is a compound represented by any one of the following formulas (211) to (213):

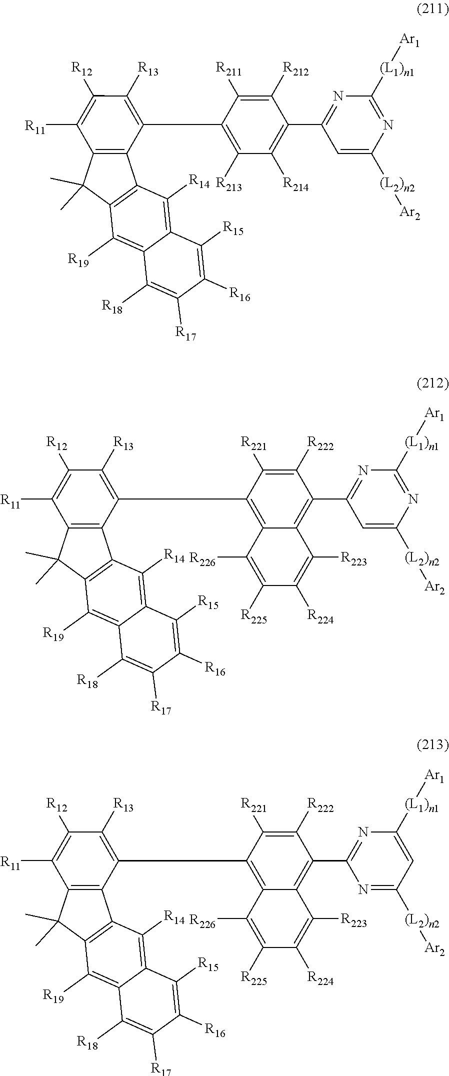

wherein in the formulas (211),
Ar₁, Ar₂, L₁, L₂, n1, n2, and R₁₁ to R₁₉ are as defined in the formula (1),
R₂₁₁ to R₂₁₄ are independently a hydrogen atom or a substituent R, and
a set of adjacent two or more of R₂₁₁ to R₂₁₄ does not bond with each other, and
the substituent R is as defined in the formula (1).

In one embodiment, the compound represented by the formula (21) is a compound represented by the formula (221) or (222):

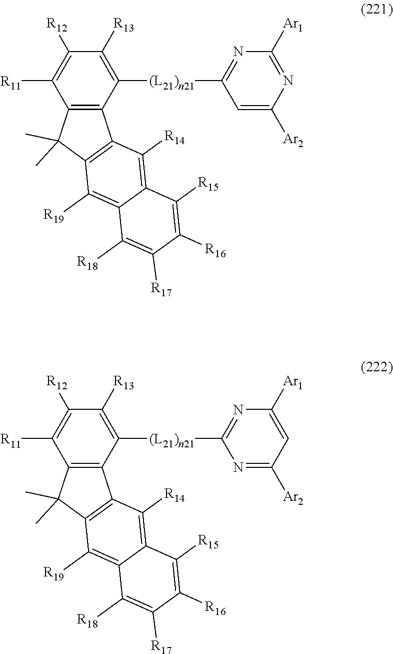

wherein in the formula (221) and the formula (222),
Ar₁, Ar₂, R₁ to R₁₉, L₂₁, and n21 are as defined in the formula (21).

In one embodiment, L₁ and L₂ in the formula (1) are independently a single bond, a substituted or unsubstituted phenylene group or a substituted or unsubstituted naphthylene group.

In one embodiment, Ar₁ and Ar₂ in the formula (1) are independently a substituted or unsubstituted aryl group including 6 to 18 ring carbon atoms, or a substituted or unsubstituted monovalent heterocyclic group including 5 to 18 ring atoms.

In one embodiment, Ar₁ and Ar₂ in the formula (1) are independently a substituted or unsubstituted phenyl group, a substituted or unsubstituted naphthyl group, or a substituted or unsubstituted biphenyl group.

In one embodiment, R₁ to R₁₉ in the formula (1) are hydrogen atoms.

In one embodiment, the substituent in the case of “substituted or unsubstituted”, and the substituent R in the formula (1) are selected from the group consisting of
an alkyl group including 1 to 50 carbon atoms,
an aryl group including 6 to 50 ring carbon atoms, and
a monovalent heterocyclic group including 5 to 50 ring atoms.

In one embodiment, the substituent in the case of “substituted or unsubstituted”, and the substituent R in the formula (1) are selected from the group consisting of
an alkyl group including 1 to 18 carbon atoms,
an aryl group including 6 to 18 ring carbon atoms, and
a monovalent heterocyclic group including 5 to 18 ring atoms.

In one embodiment, a compound according to one aspect of the invention has no deuterium atom in the molecular as a hydrogen atom.

In the present specification, “having no deuterium atom as a hydrogen atom” means that the proportion of deuterium atoms to the sum of protium atoms and deuterium atoms in all hydrogen atoms contained in the molecular is less than or equal to the natural abundance ratio of deuterium atoms. In other words, the compound of the invention that does not have a deuterium atom in the molecular as a hydrogen atom may contain deuterium atom in a proportion equal to or less than the natural abundance ratio.

The fact that the proportion of deuterium atoms to the sum of protium atoms and deuterium atoms is less than or equal to the natural abundance ratio, can be confirmed by nuclear magnetic resonance apparatus.

In one embodiment, the compound according to one aspect of the invention has at least one deuterium atom in the molecule as a hydrogen atom.

In present specification, “having a deuterium atom as a hydrogen atom” means that the proportion of deuterium atoms to the sum of protium atoms and deuterium atoms is greater than or equal to the natural abundance ratio of deuterium atoms. The fact that the proportion of deuterium atoms to the sum of protium atoms and deuterium atoms is greater than the natural abundance ratio, can be confirmed by nuclear magnetic resonance apparatus.

In one embodiment, at least one hydrogen atom out of the followings is a deuterium atom:
H of X₁ which is CH,
H of X₂ which is CH,
hydrogen atoms possessed by Ar₁,
hydrogen atoms possessed by Ar₂,
hydrogen atoms possessed by L₁,
hydrogen atoms possessed by L₂,
hydrogen atoms possessed by L₃,
R₁₁ to R₁₉ which are hydrogen atoms,
hydrogen atoms possessed by R₁ to R₁₉ which are the substituent R,
R₂₁ and R₂₂ which are hydrogen atoms,
hydrogen atoms possessed by R₂₁ and R₂₂ which are the substituent R, and
hydrogen atoms possessed by the substituted or unsubstituted saturated or unsaturated ring which is formed by R₂₁ and R₂₂.

In one embodiment, at least one of the hydrogen atoms possessed by Ar₁ in the formula (1) is a deuterium atom.

The compound represented by the formula (1) can be synthesized in accordance with Examples by using a known alternative reaction or raw material to a desired product.

Specific examples of the compound represented by the formula (1) are described below. However, these are merely examples, and the compound represented by the formula (1) is not limited to the following specific examples.

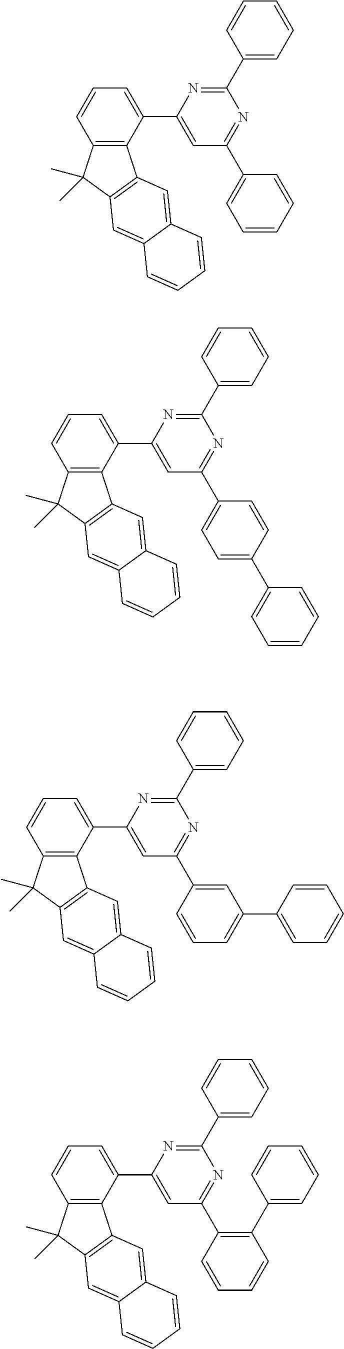 [SMILES: CC1(C)c2cc3ccccc3cc2-c2c(-c3cc(-c4ccccc4)nc(-c4ccccc4)n3)cccc21 ; CC1(C)c2cc3ccccc3cc2-c2c(-c3cc(-c4ccc(-c5ccccc5)cc4)nc(-c4ccccc4)n3)cccc21 ; CC1(C)c2cc3ccccc3cc2-c2c(-c3cc(-c4cccc(-c5ccccc5)c4)nc(-c4ccccc4)n3)cccc21 ; CC1(C)c2cc3ccccc3cc2-c2c(-c3cc(-c4ccccc4-c4ccccc4)nc(-c4ccccc4)n3)cccc21]

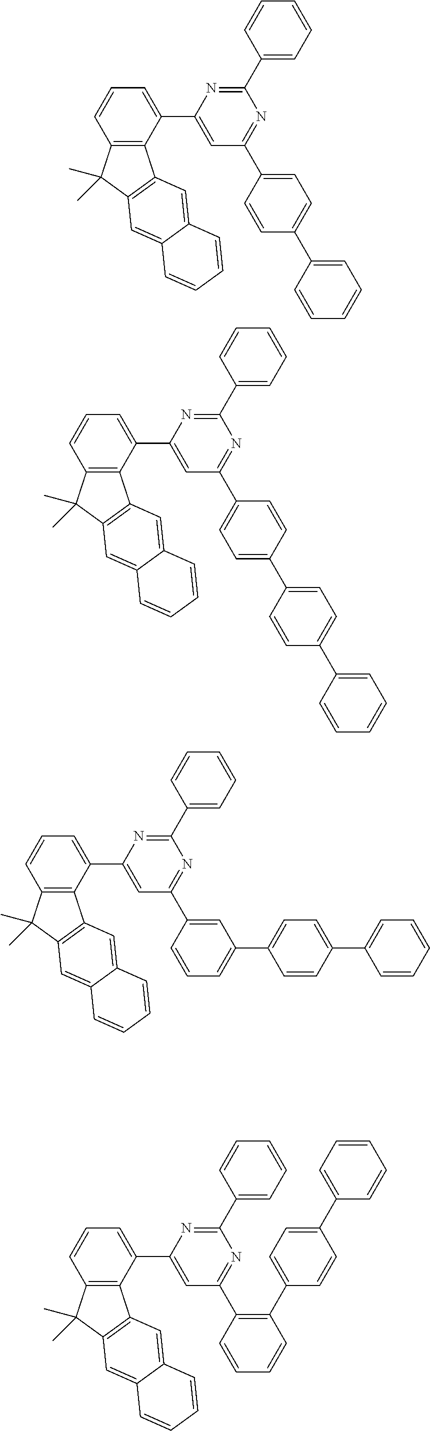

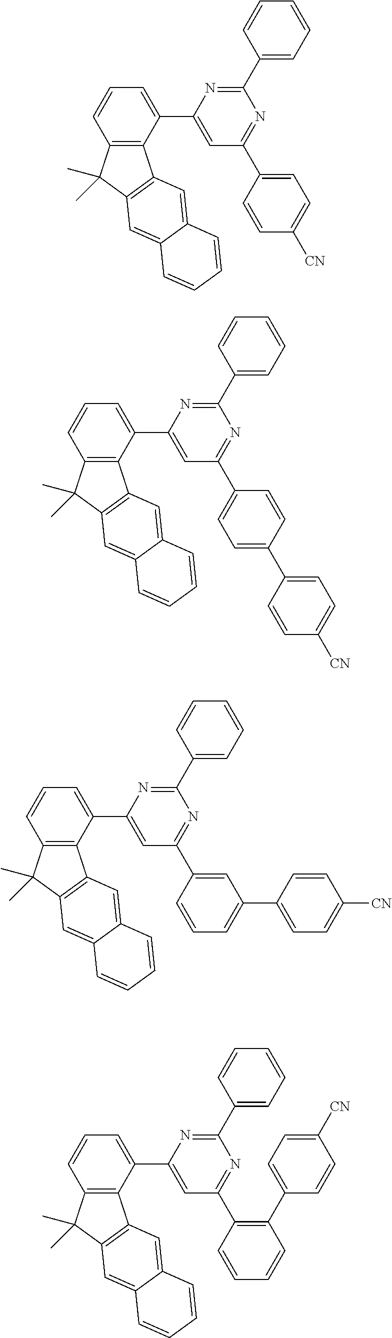 [SMILES: CC1(C)c2cc3ccccc3cc2-c2c(-c3cc(-c4ccc(C#N)cc4)nc(-c4ccccc4)n3)cccc21 ; CC1(C)c2cc3ccccc3cc2-c2c(-c3cc(-c4ccc(-c5ccc(C#N)cc5)cc4)nc(-c4ccccc4)n3)cccc21 ; CC1(C)c2cc3ccccc3cc2-c2c(-c3cc(-c4cccc(-c5ccc(C#N)cc5)c4)nc(-c4ccccc4)n3)cccc21 ; CC1(C)c2cc3ccccc3cc2-c2c(-c3cc(-c4ccccc4-c4ccc(C#N)cc4)nc(-c4ccccc4)n3)cccc21]

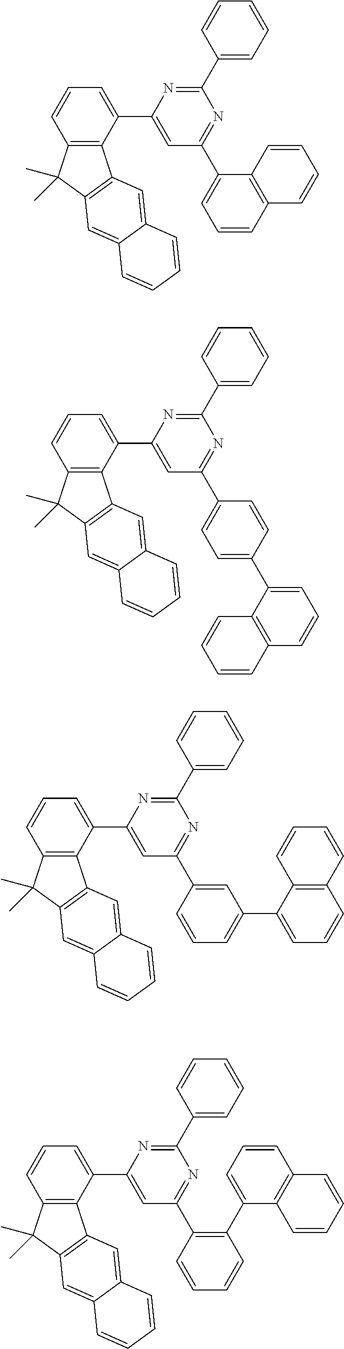

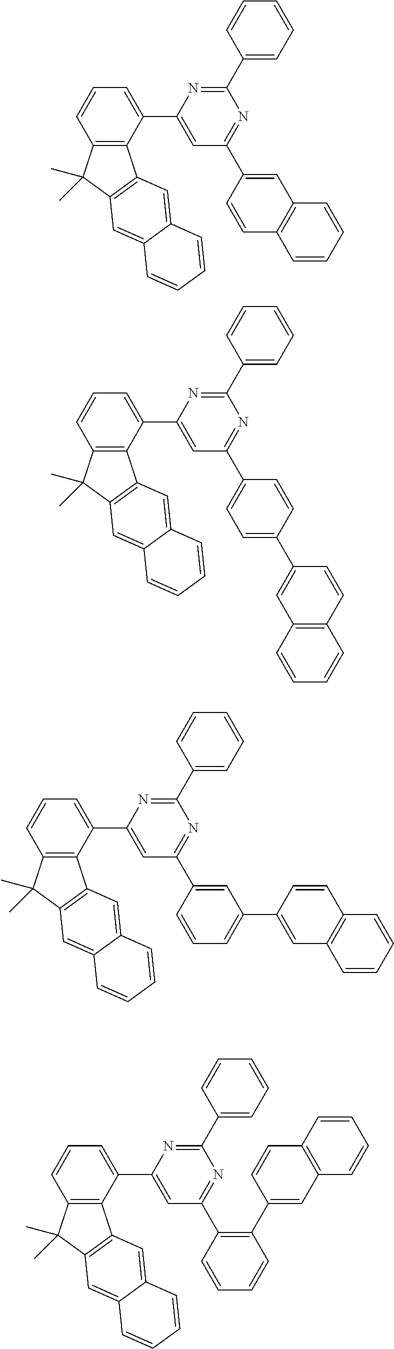 [SMILES: CC1(C)c2cc3ccccc3cc2-c2c(-c3cc(-c4ccc5ccccc5c4)nc(-c4ccccc4)n3)cccc21 ; CC1(C)c2cc3ccccc3cc2-c2c(-c3cc(-c4ccc(-c5ccc6ccccc6c5)cc4)nc(-c4ccccc4)n3)cccc21 ; CC1(C)c2cc3ccccc3cc2-c2c(-c3cc(-c4cccc(-c5ccc6ccccc6c5)c4)nc(-c4ccccc4)n3)cccc21 ; CC1(C)c2cc3ccccc3cc2-c2c(-c3cc(-c4ccccc4-c4ccc5ccccc5c4)nc(-c4ccccc4)n3)cccc21]

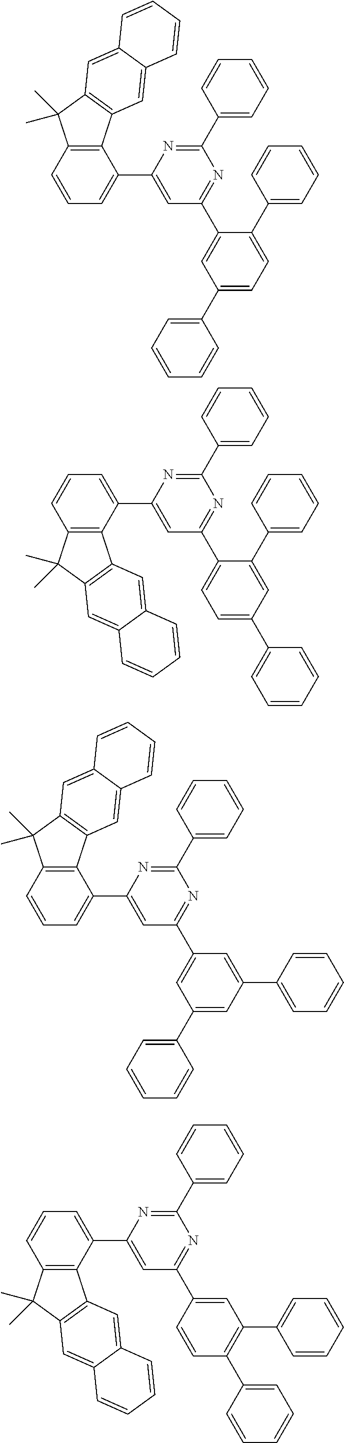

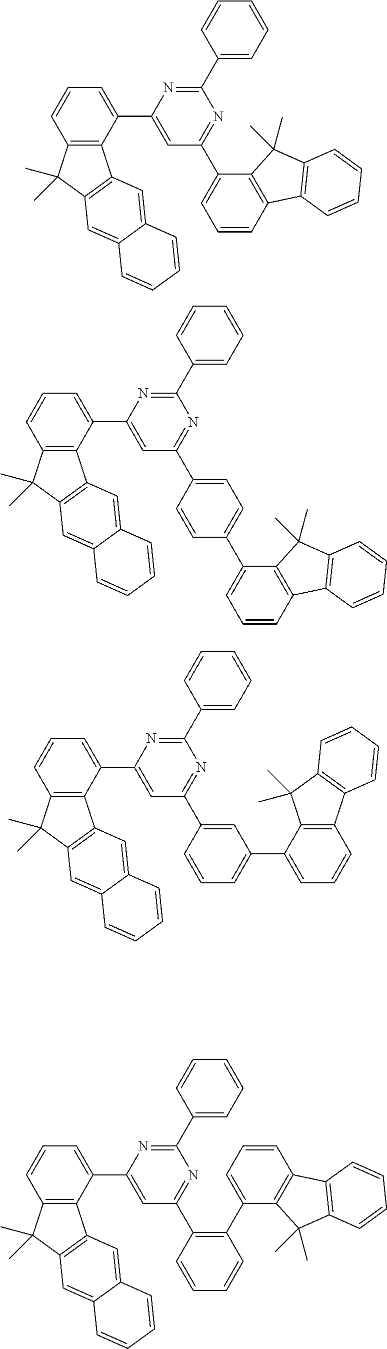

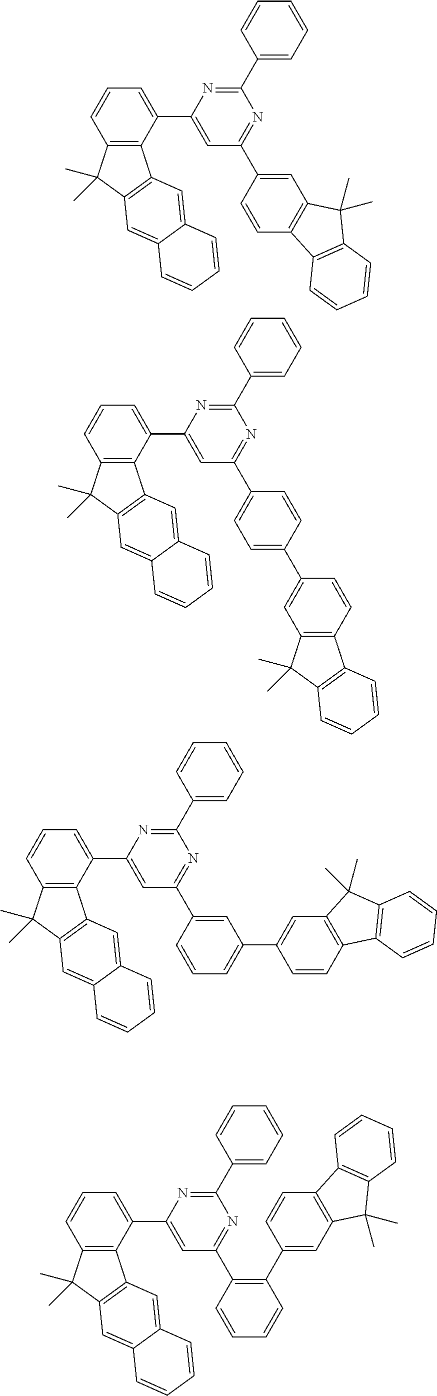

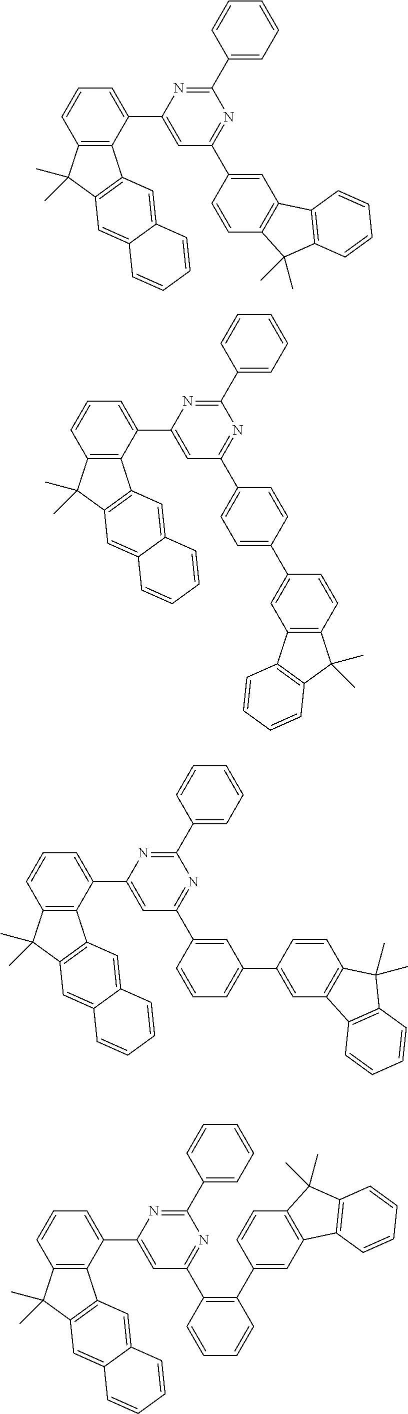

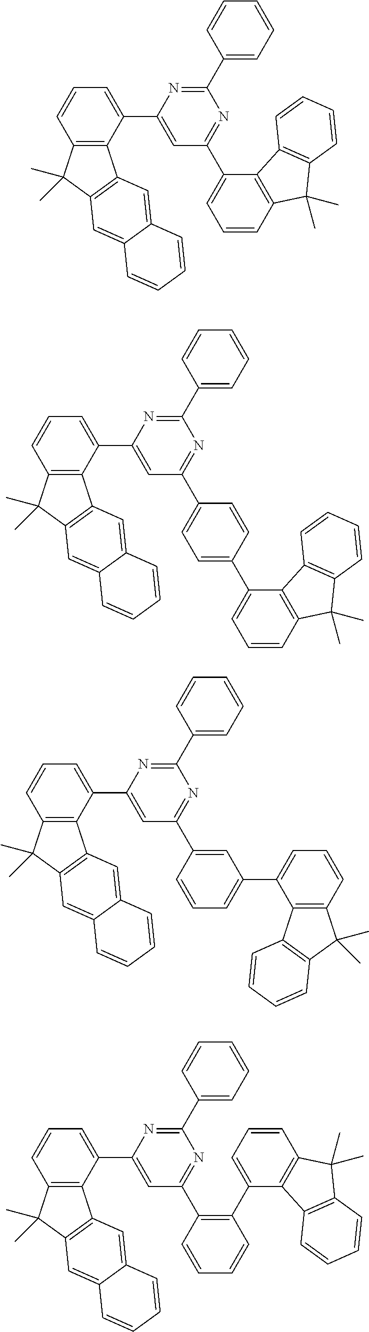

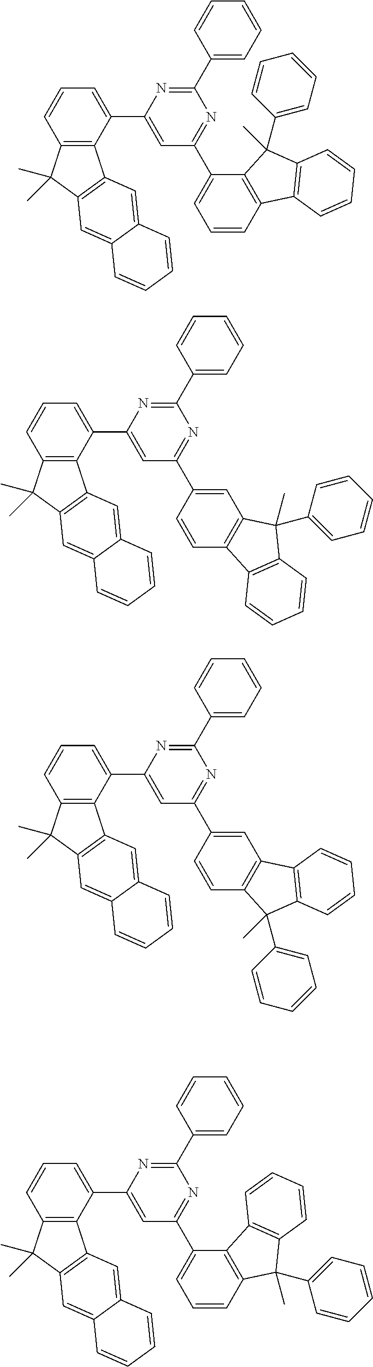

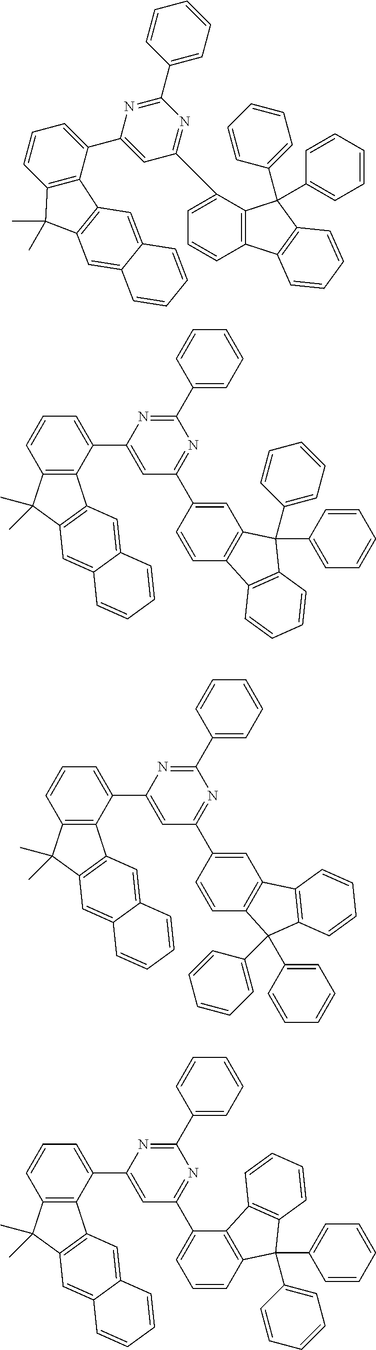

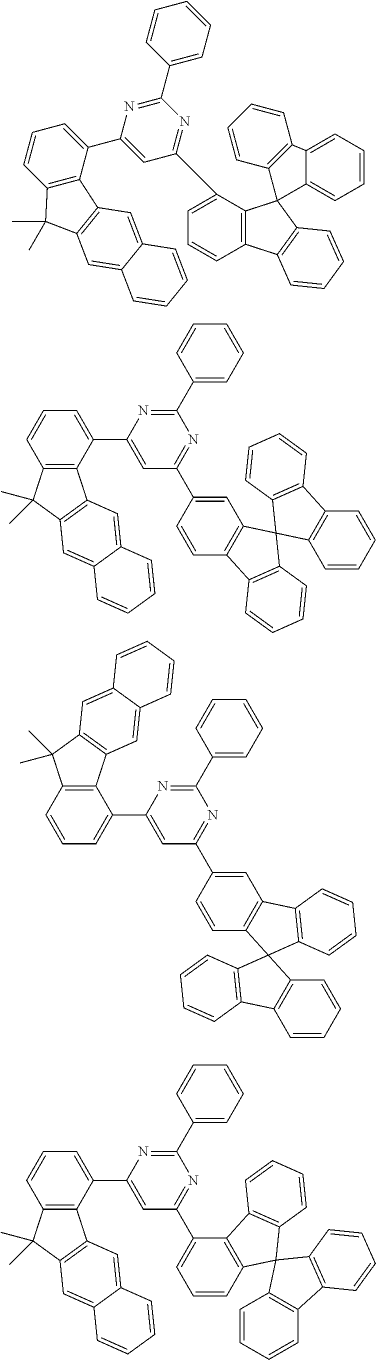

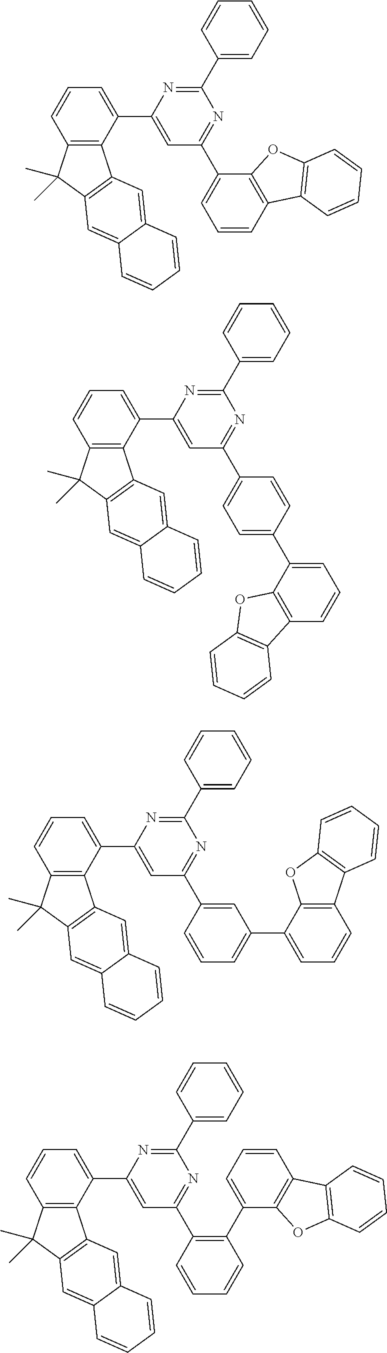

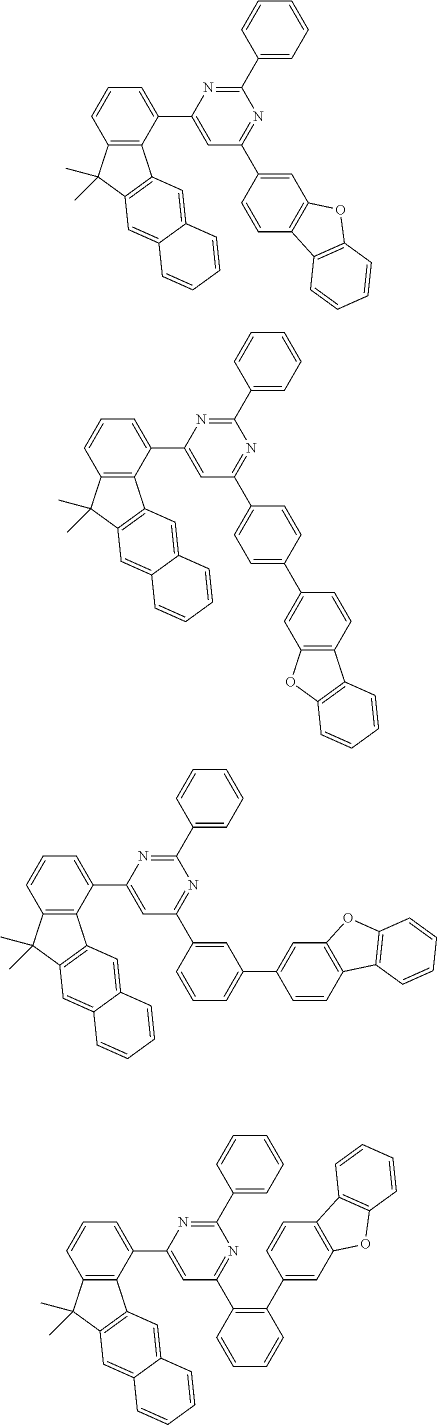 [SMILES: CC1(C)c2cc3ccccc3cc2-c2c(-c3cc(-c4ccc5c(c4)oc4ccccc45)nc(-c4ccccc4)n3)cccc21 ; CC1(C)c2cc3ccccc3cc2-c2c(-c3cc(-c4ccc(-c5ccc6c(c5)oc5ccccc56)cc4)nc(-c4ccccc4)n3)cccc21 ; CC1(C)c2cc3ccccc3cc2-c2c(-c3cc(-c4cccc(-c5ccc6c(c5)oc5ccccc56)c4)nc(-c4ccccc4)n3)cccc21 ; CC1(C)c2cc3ccccc3cc2-c2c(-c3cc(-c4ccccc4-c4ccc5c(c4)oc4ccccc45)nc(-c4ccccc4)n3)cccc21]

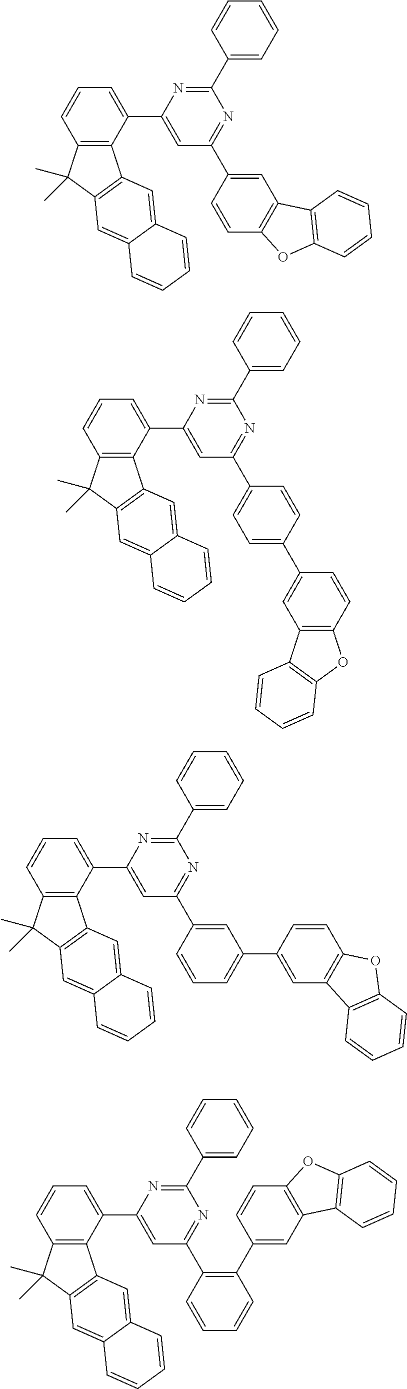 [SMILES: CC1(C)c2cc3ccccc3cc2-c2c(-c3cc(-c4ccc5oc6ccccc6c5c4)nc(-c4ccccc4)n3)cccc21 ; CC1(C)c2cc3ccccc3cc2-c2c(-c3cc(-c4ccc(-c5ccc6oc7ccccc7c6c5)cc4)nc(-c4ccccc4)n3)cccc21 ; CC1(C)c2cc3ccccc3cc2-c2c(-c3cc(-c4cccc(-c5ccc6oc7ccccc7c6c5)c4)nc(-c4ccccc4)n3)cccc21 ; CC1(C)c2cc3ccccc3cc2-c2c(-c3cc(-c4ccccc4-c4ccc5oc6ccccc6c5c4)nc(-c4ccccc4)n3)cccc21]

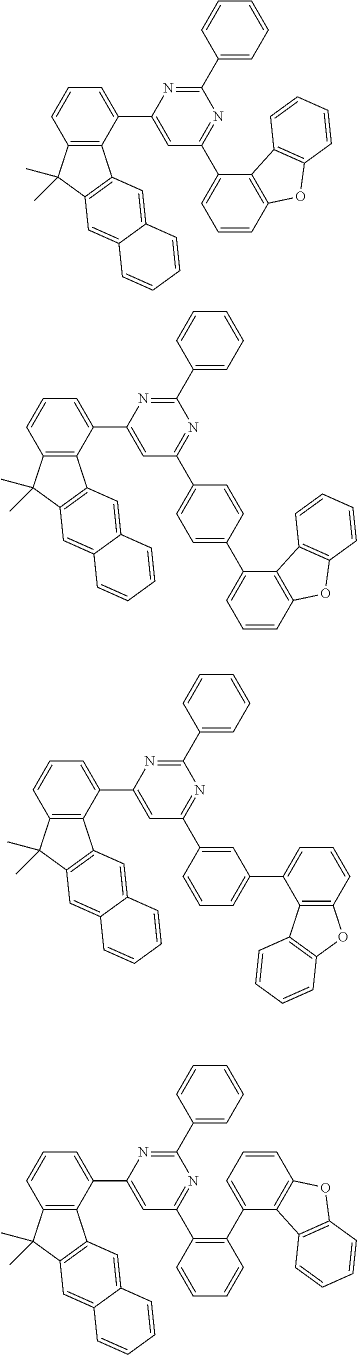

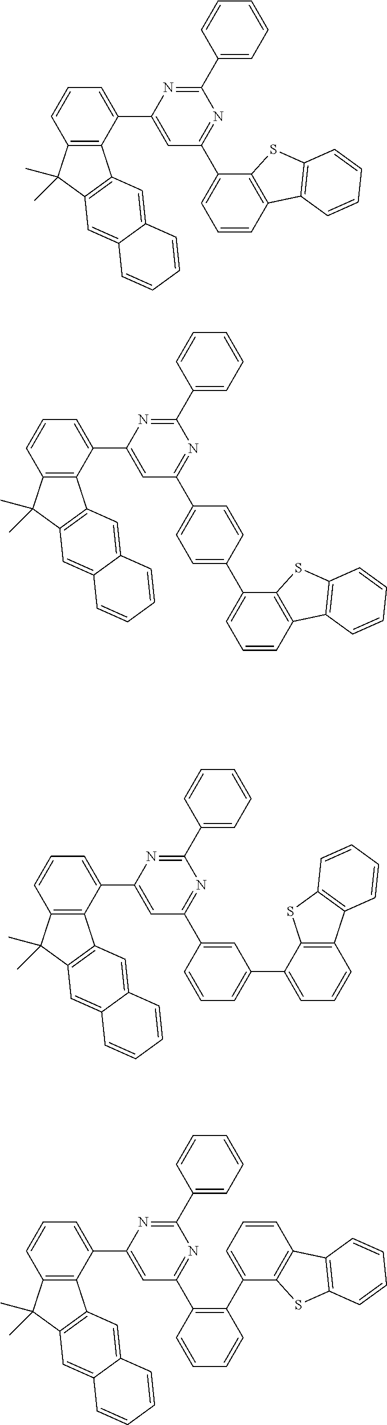 [SMILES: CC1(C)c2cc3ccccc3cc2-c2c(-c3cc(-c4cccc5c4sc4ccccc45)nc(-c4ccccc4)n3)cccc21 ; CC1(C)c2cc3ccccc3cc2-c2c(-c3cc(-c4ccc(-c5cccc6c5sc5ccccc56)cc4)nc(-c4ccccc4)n3)cccc21 ; CC1(C)c2cc3ccccc3cc2-c2c(-c3cc(-c4cccc(-c5cccc6c5sc5ccccc56)c4)nc(-c4ccccc4)n3)cccc21 ; CC1(C)c2cc3ccccc3cc2-c2c(-c3cc(-c4ccccc4-c4cccc5c4sc4ccccc45)nc(-c4ccccc4)n3)cccc21]

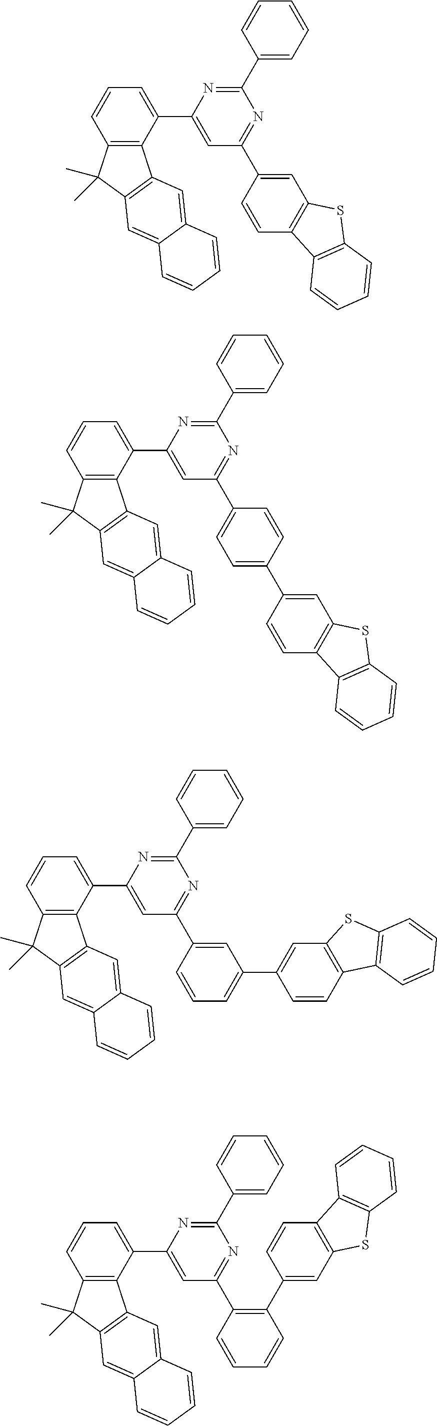 [SMILES: CC1(C)c2cc3ccccc3cc2-c2c(-c3cc(-c4ccc5c(c4)sc4ccccc45)nc(-c4ccccc4)n3)cccc21 ; CC1(C)c2cc3ccccc3cc2-c2c(-c3cc(-c4ccc(-c5ccc6c(c5)sc5ccccc56)cc4)nc(-c4ccccc4)n3)cccc21 ; CC1(C)c2cc3ccccc3cc2-c2c(-c3cc(-c4cccc(-c5ccc6c(c5)sc5ccccc56)c4)nc(-c4ccccc4)n3)cccc21 ; CC1(C)c2cc3ccccc3cc2-c2c(-c3cc(-c4ccccc4-c4ccc5c(c4)sc4ccccc45)nc(-c4ccccc4)n3)cccc21]

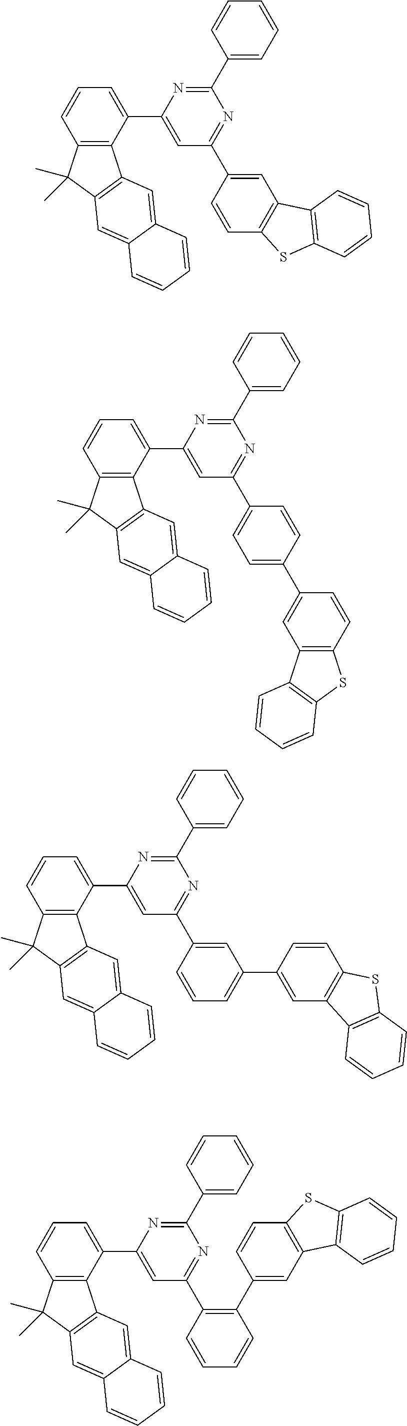 [SMILES: CC1(C)c2cc3ccccc3cc2-c2c(-c3cc(-c4ccc5sc6ccccc6c5c4)nc(-c4ccccc4)n3)cccc21 ; CC1(C)c2cc3ccccc3cc2-c2c(-c3cc(-c4ccc(-c5ccc6sc7ccccc7c6c5)cc4)nc(-c4ccccc4)n3)cccc21 ; CC1(C)c2cc3ccccc3cc2-c2c(-c3cc(-c4cccc(-c5ccc6sc7ccccc7c6c5)c4)nc(-c4ccccc4)n3)cccc21 ; CC1(C)c2cc3ccccc3cc2-c2c(-c3cc(-c4ccccc4-c4ccc5sc6ccccc6c5c4)nc(-c4ccccc4)n3)cccc21]

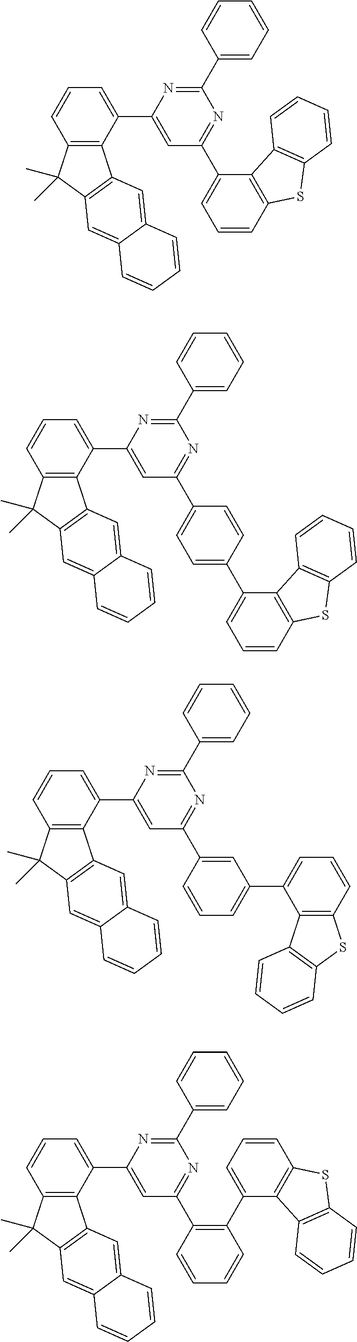

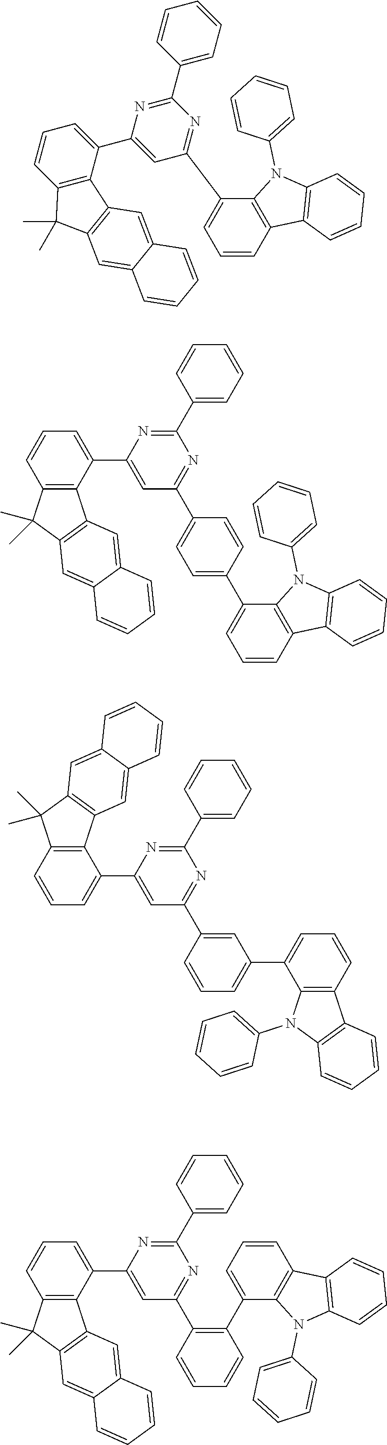

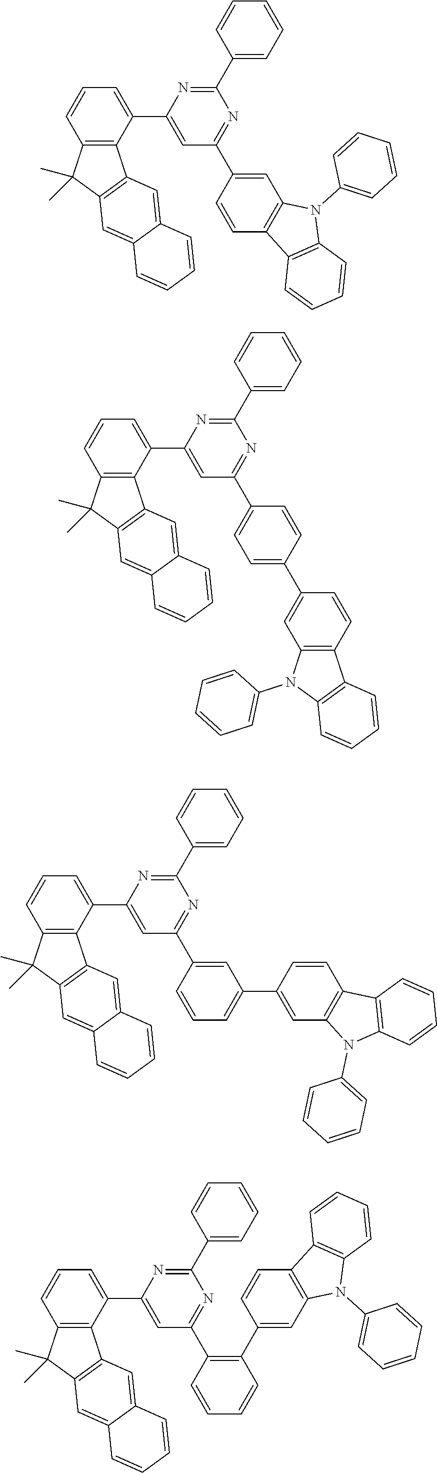

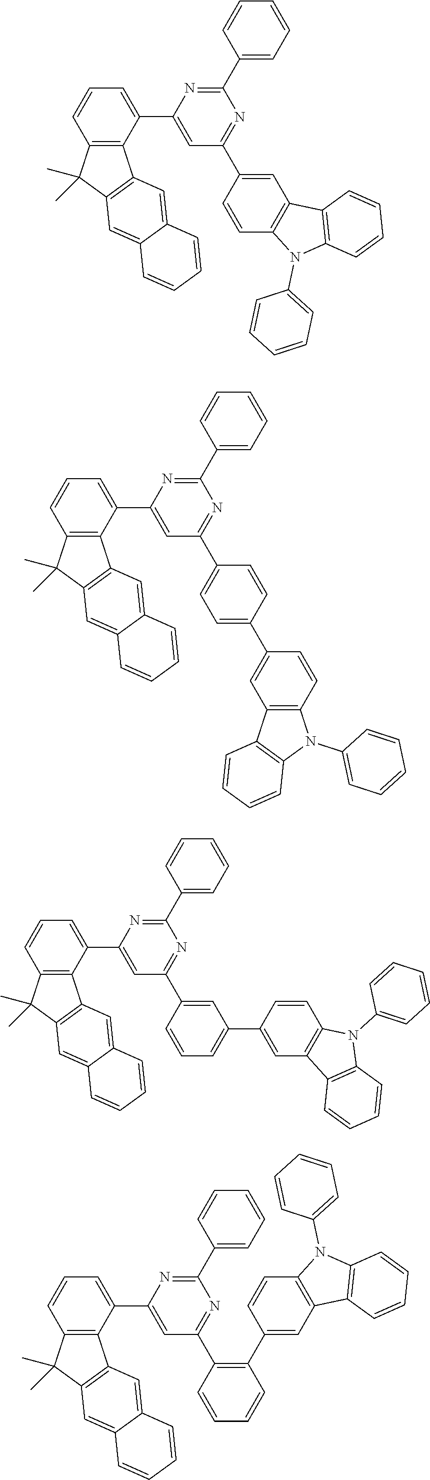

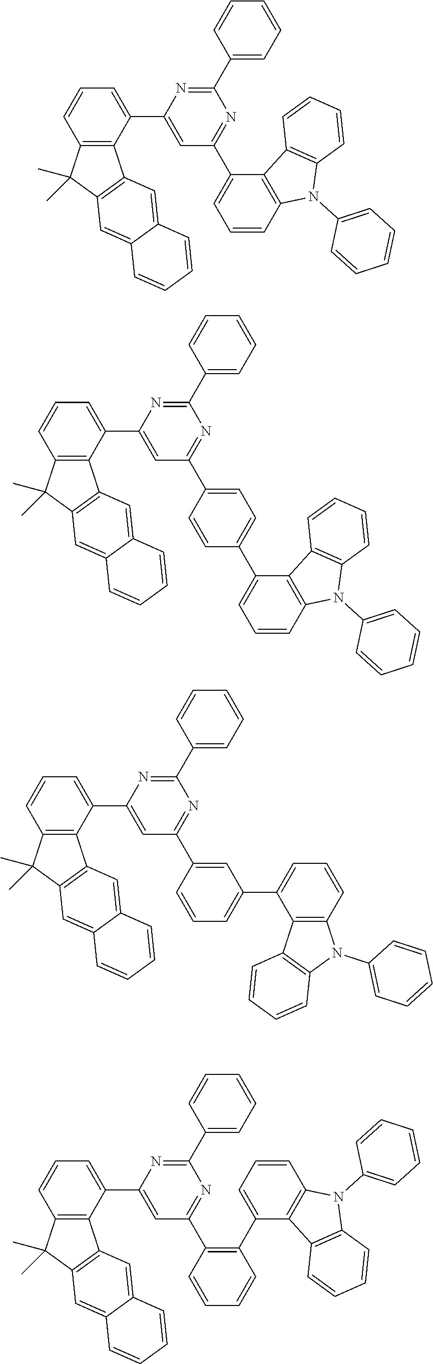 [SMILES: CC1(C)c2cc3ccccc3cc2-c2c(-c3cc(-c4cccc5c4c4ccccc4n5-c4ccccc4)nc(-c4ccccc4)n3)cccc21 ; CC1(C)c2cc3ccccc3cc2-c2c(-c3cc(-c4ccc(-c5cccc6c5c5ccccc5n6-c5ccccc5)cc4)nc(-c4ccccc4)n3)cccc21 ; CC1(C)c2cc3ccccc3cc2-c2c(-c3cc(-c4cccc(-c5cccc6c5c5ccccc5n6-c5ccccc5)c4)nc(-c4ccccc4)n3)cccc21 ; CC1(C)c2cc3ccccc3cc2-c2c(-c3cc(-c4ccccc4-c4cccc5c4c4ccccc4n5-c4ccccc4)nc(-c4ccccc4)n3)cccc21]

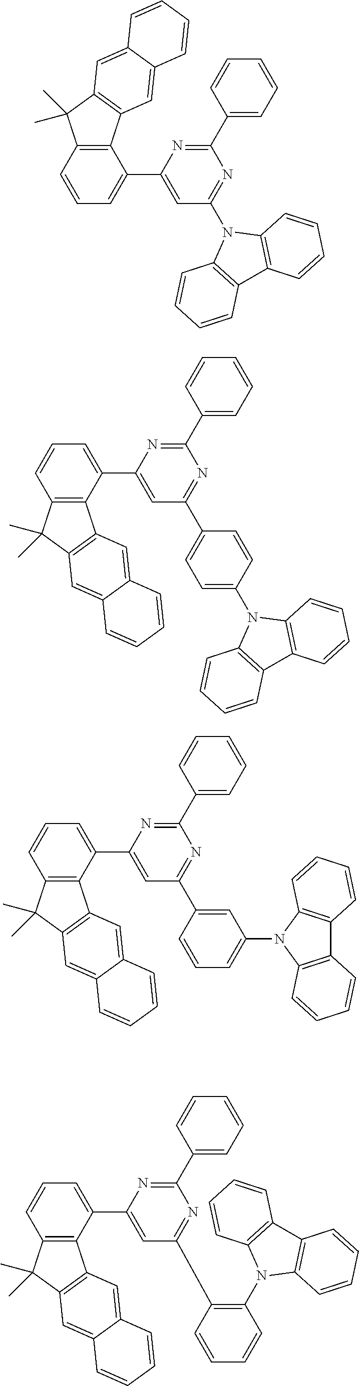

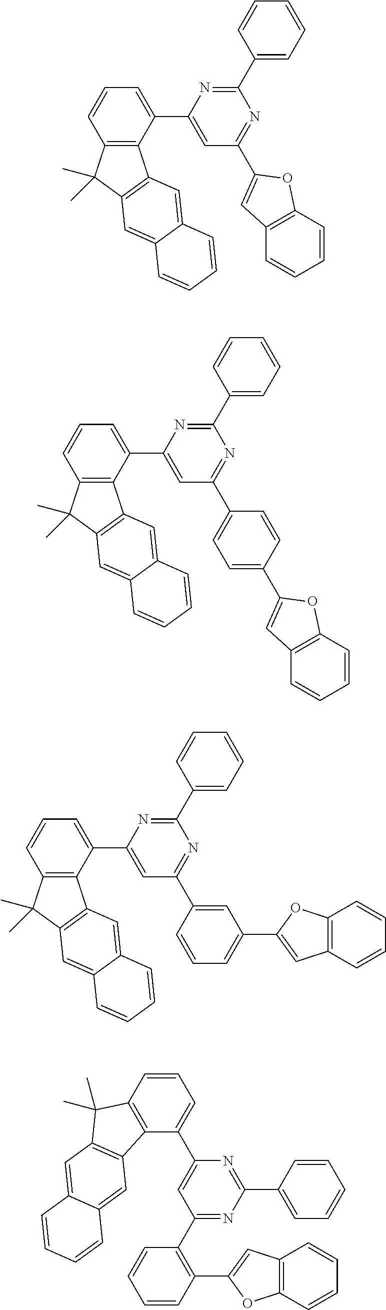 [SMILES: CC1(C)c2cc3ccccc3cc2-c2c(-c3cc(-c4cc5ccccc5o4)nc(-c4ccccc4)n3)cccc21 ; CC1(C)c2cc3ccccc3cc2-c2c(-c3cc(-c4ccc(-c5cc6ccccc6o5)cc4)nc(-c4ccccc4)n3)cccc21 ; CC1(C)c2cc3ccccc3cc2-c2c(-c3cc(-c4cccc(-c5cc6ccccc6o5)c4)nc(-c4ccccc4)n3)cccc21 ; CC1(C)c2cc3ccccc3cc2-c2c(-c3cc(-c4ccccc4-c4cc5ccccc5o4)nc(-c4ccccc4)n3)cccc21]

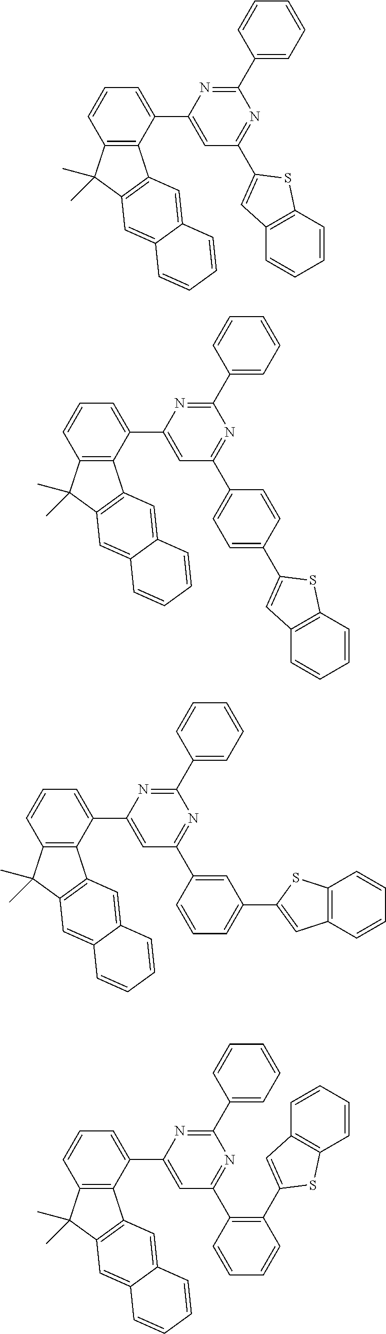 [SMILES: CC1(C)c2cc3ccccc3cc2-c2c(-c3cc(-c4cc5ccccc5s4)nc(-c4ccccc4)n3)cccc21 ; CC1(C)c2cc3ccccc3cc2-c2c(-c3cc(-c4ccc(-c5cc6ccccc6s5)cc4)nc(-c4ccccc4)n3)cccc21 ; CC1(C)c2cc3ccccc3cc2-c2c(-c3cc(-c4cccc(-c5cc6ccccc6s5)c4)nc(-c4ccccc4)n3)cccc21 ; CC1(C)c2cc3ccccc3cc2-c2c(-c3cc(-c4ccccc4-c4cc5ccccc5s4)nc(-c4ccccc4)n3)cccc21]

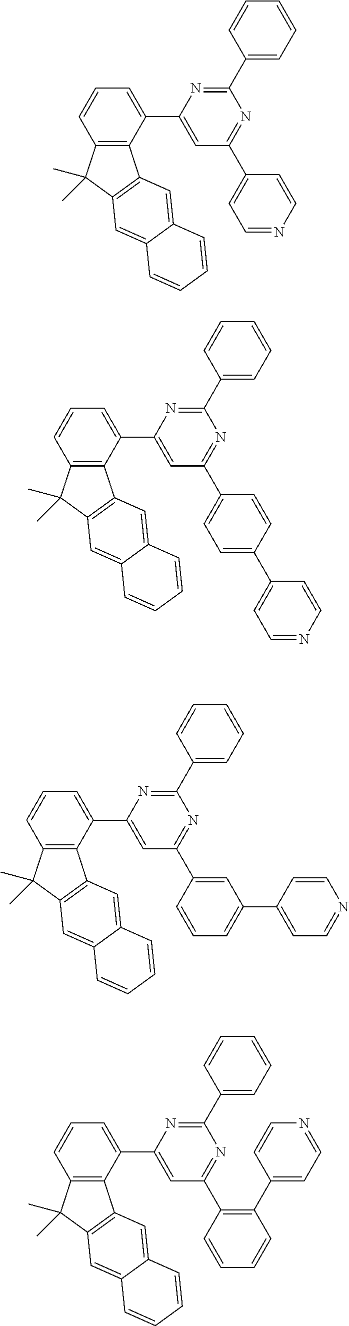 [SMILES: CC1(C)c2cc3ccccc3cc2-c2c(-c3cc(-c4ccncc4)nc(-c4ccccc4)n3)cccc21 ; CC1(C)c2cc3ccccc3cc2-c2c(-c3cc(-c4ccc(-c5ccncc5)cc4)nc(-c4ccccc4)n3)cccc21 ; CC1(C)c2cc3ccccc3cc2-c2c(-c3cc(-c4cccc(-c5ccncc5)c4)nc(-c4ccccc4)n3)cccc21 ; CC1(C)c2cc3ccccc3cc2-c2c(-c3cc(-c4ccccc4-c4ccncc4)nc(-c4ccccc4)n3)cccc21]

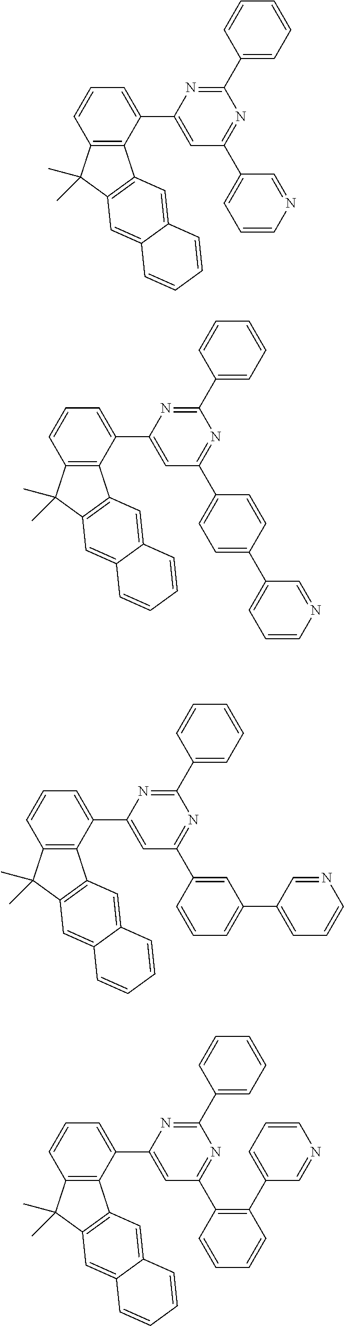 [SMILES: CC1(C)c2cc3ccccc3cc2-c2c(-c3cc(-c4cccnc4)nc(-c4ccccc4)n3)cccc21 ; CC1(C)c2cc3ccccc3cc2-c2c(-c3cc(-c4ccc(-c5cccnc5)cc4)nc(-c4ccccc4)n3)cccc21 ; CC1(C)c2cc3ccccc3cc2-c2c(-c3cc(-c4cccc(-c5cccnc5)c4)nc(-c4ccccc4)n3)cccc21 ; CC1(C)c2cc3ccccc3cc2-c2c(-c3cc(-c4ccccc4-c4cccnc4)nc(-c4ccccc4)n3)cccc21]

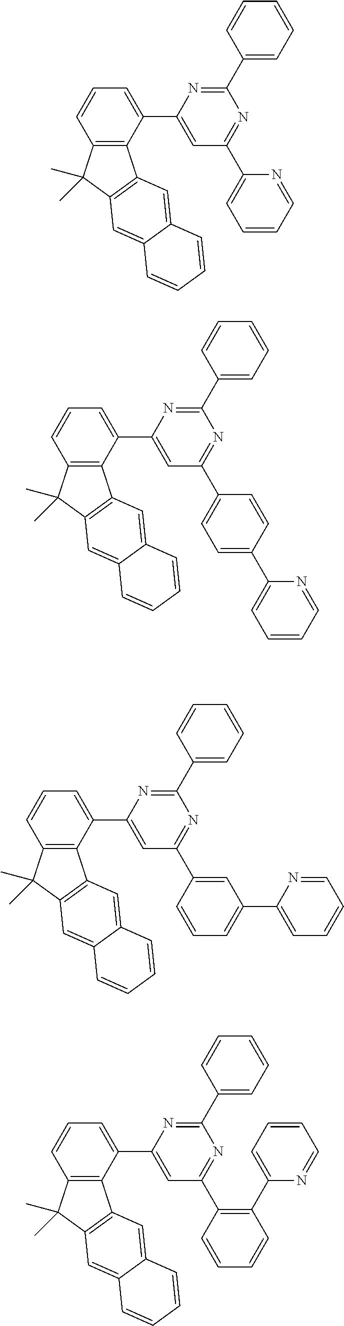 [SMILES: CC1(C)c2cc3ccccc3cc2-c2c(-c3cc(-c4ccccn4)nc(-c4ccccc4)n3)cccc21 ; CC1(C)c2cc3ccccc3cc2-c2c(-c3cc(-c4ccc(-c5ccccn5)cc4)nc(-c4ccccc4)n3)cccc21 ; CC1(C)c2cc3ccccc3cc2-c2c(-c3cc(-c4cccc(-c5ccccn5)c4)nc(-c4ccccc4)n3)cccc21 ; CC1(C)c2cc3ccccc3cc2-c2c(-c3cc(-c4ccccc4-c4ccccn4)nc(-c4ccccc4)n3)cccc21]

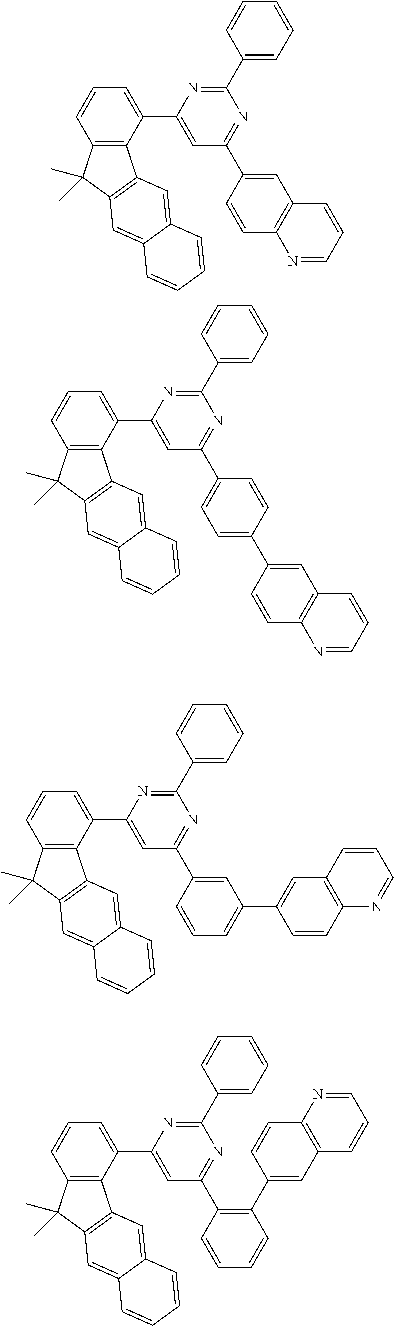

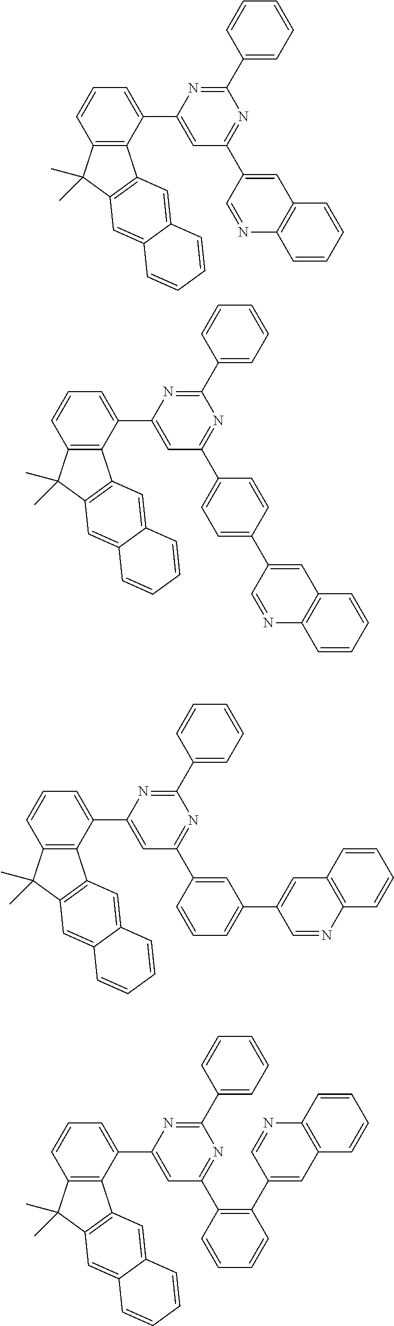

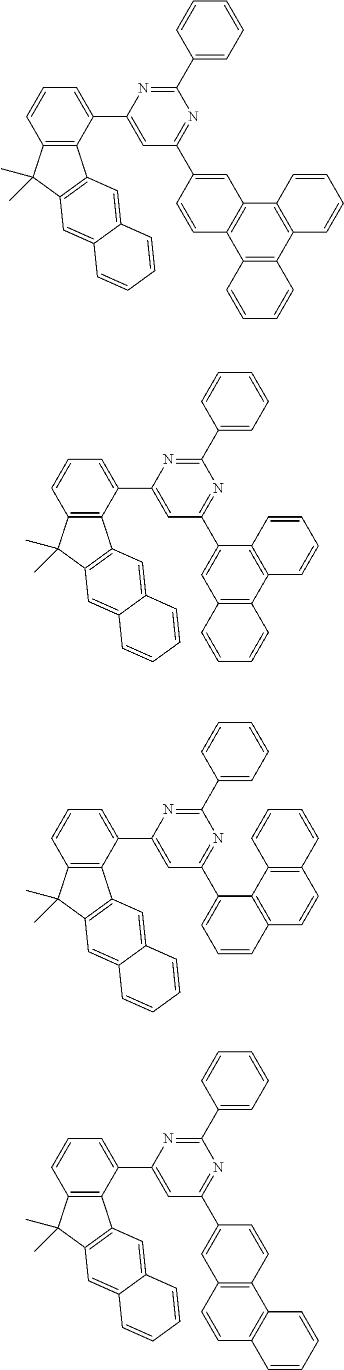 [SMILES: CC1(C)c2cc3ccccc3cc2-c2c(-c3cc(-c4ccc5c6ccccc6c6ccccc6c5c4)nc(-c4ccccc4)n3)cccc21 ; CC1(C)c2cc3ccccc3cc2-c2c(-c3cc(-c4cc5ccccc5c5ccccc45)nc(-c4ccccc4)n3)cccc21 ; CC1(C)c2cc3ccccc3cc2-c2c(-c3cc(-c4cccc5ccc6ccccc6c45)nc(-c4ccccc4)n3)cccc21 ; CC1(C)c2cc3ccccc3cc2-c2c(-c3cc(-c4ccc5c(ccc6ccccc65)c4)nc(-c4ccccc4)n3)cccc21]

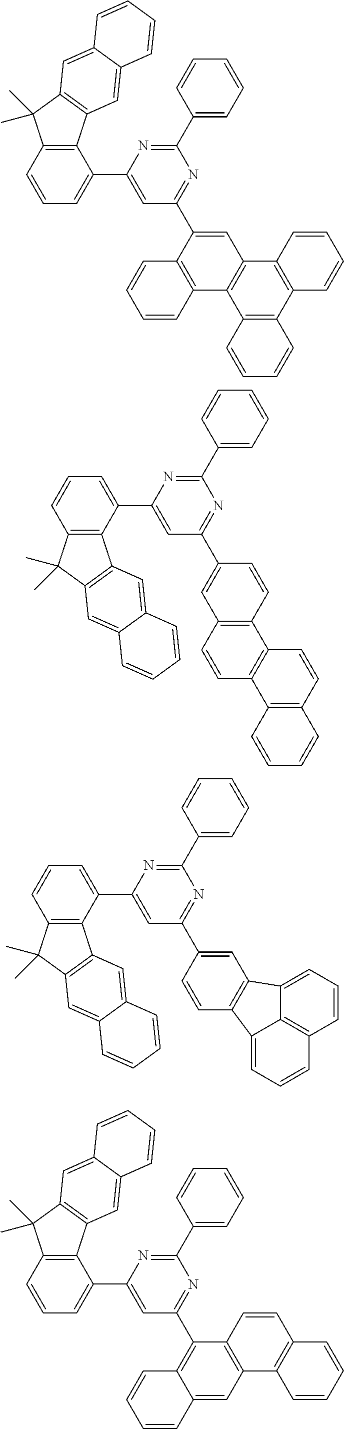

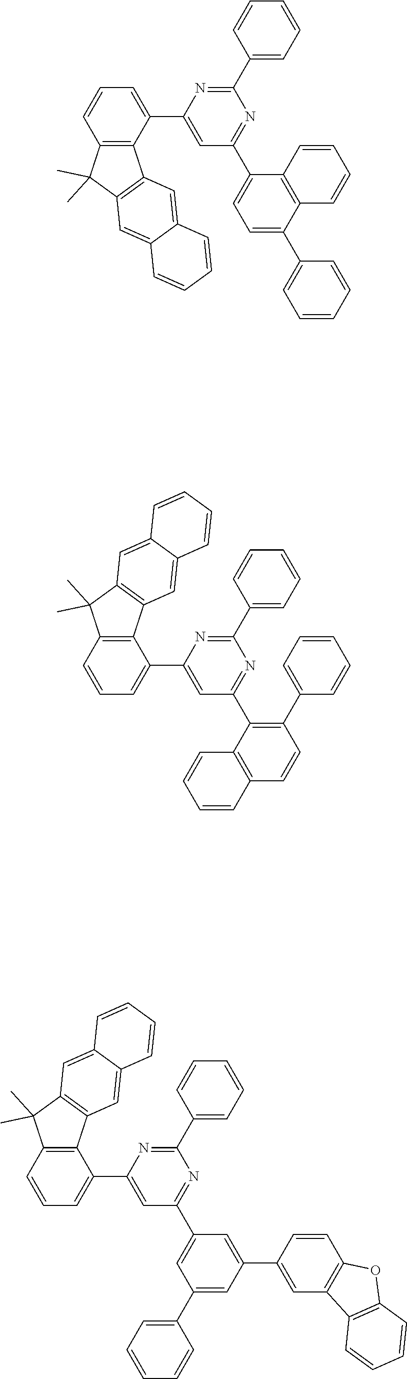 [SMILES: CC1(C)c2cc3ccccc3cc2-c2c(-c3cc(-c4ccc(-c5ccccc5)c5ccccc45)nc(-c4ccccc4)n3)cccc21 ; CC1(C)c2cc3ccccc3cc2-c2c(-c3cc(-c4c(-c5ccccc5)ccc5ccccc45)nc(-c4ccccc4)n3)cccc21 ; CC1(C)c2cc3ccccc3cc2-c2c(-c3cc(-c4cc(-c5ccccc5)cc(-c5ccc6oc7ccccc7c6c5)c4)nc(-c4ccccc4)n3)cccc21]

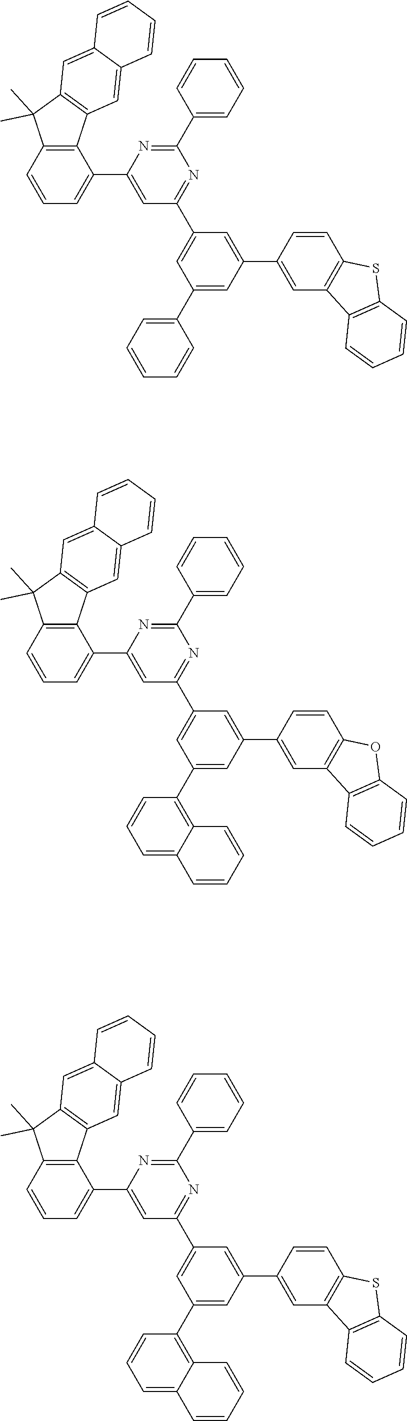 [SMILES: CC1(C)c2cc3ccccc3cc2-c2c(-c3cc(-c4cc(-c5ccccc5)cc(-c5ccc6sc7ccccc7c6c5)c4)nc(-c4ccccc4)n3)cccc21 ; CC1(C)c2cc3ccccc3cc2-c2c(-c3cc(-c4cc(-c5ccc6oc7ccccc7c6c5)cc(-c5cccc6ccccc56)c4)nc(-c4ccccc4)n3)cccc21 ; CC1(C)c2cc3ccccc3cc2-c2c(-c3cc(-c4cc(-c5ccc6sc7ccccc7c6c5)cc(-c5cccc6ccccc56)c4)nc(-c4ccccc4)n3)cccc21]

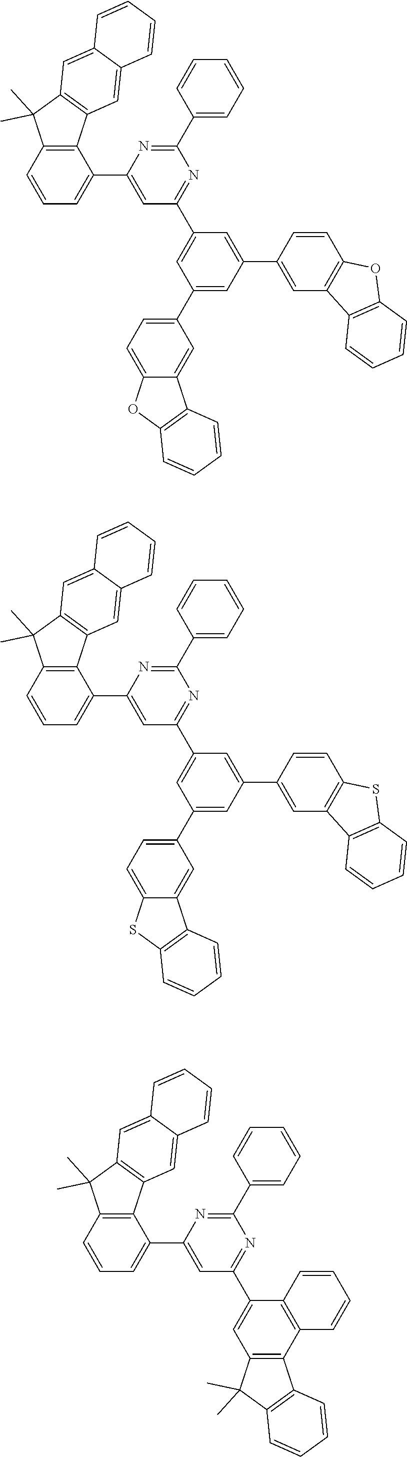 [SMILES: CC1(C)c2cc3ccccc3cc2-c2c(-c3cc(-c4cc(-c5ccc6oc7ccccc7c6c5)cc(-c5ccc6oc7ccccc7c6c5)c4)nc(-c4ccccc4)n3)cccc21 ; CC1(C)c2cc3ccccc3cc2-c2c(-c3cc(-c4cc(-c5ccc6sc7ccccc7c6c5)cc(-c5ccc6sc7ccccc7c6c5)c4)nc(-c4ccccc4)n3)cccc21 ; CC1(C)c2cc3ccccc3cc2-c2c(-c3cc(-c4cc5c(c6ccccc46)-c4ccccc4C5(C)C)nc(-c4ccccc4)n3)cccc21]

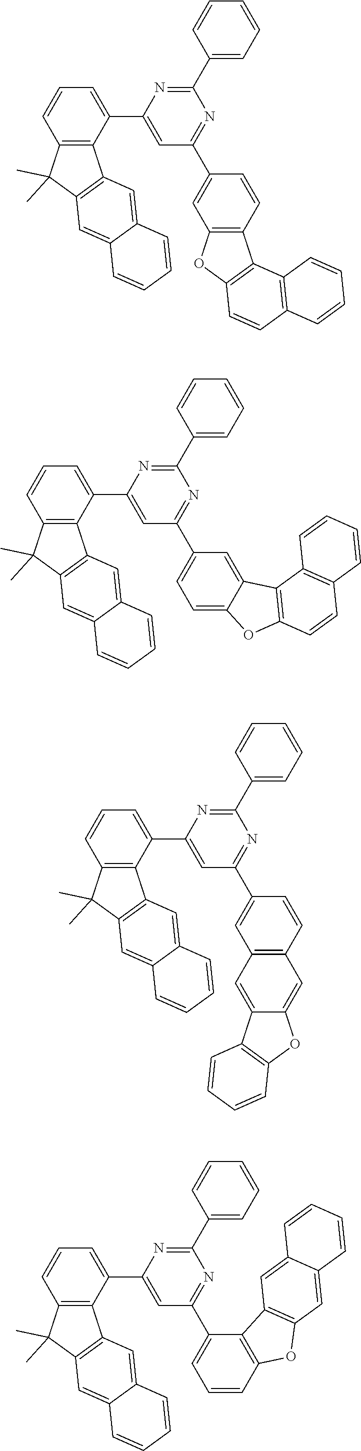 [SMILES: CC1(C)c2cc3ccccc3cc2-c2c(-c3cc(-c4ccc5c(c4)oc4ccc6ccccc6c45)nc(-c4ccccc4)n3)cccc21 ; CC1(C)c2cc3ccccc3cc2-c2c(-c3cc(-c4ccc5oc6ccc7ccccc7c6c5c4)nc(-c4ccccc4)n3)cccc21 ; CC1(C)c2cc3ccccc3cc2-c2c(-c3cc(-c4ccc5cc6oc7ccccc7c6cc5c4)nc(-c4ccccc4)n3)cccc21 ; CC1(C)c2cc3ccccc3cc2-c2c(-c3cc(-c4cccc5oc6cc7ccccc7cc6c45)nc(-c4ccccc4)n3)cccc21]

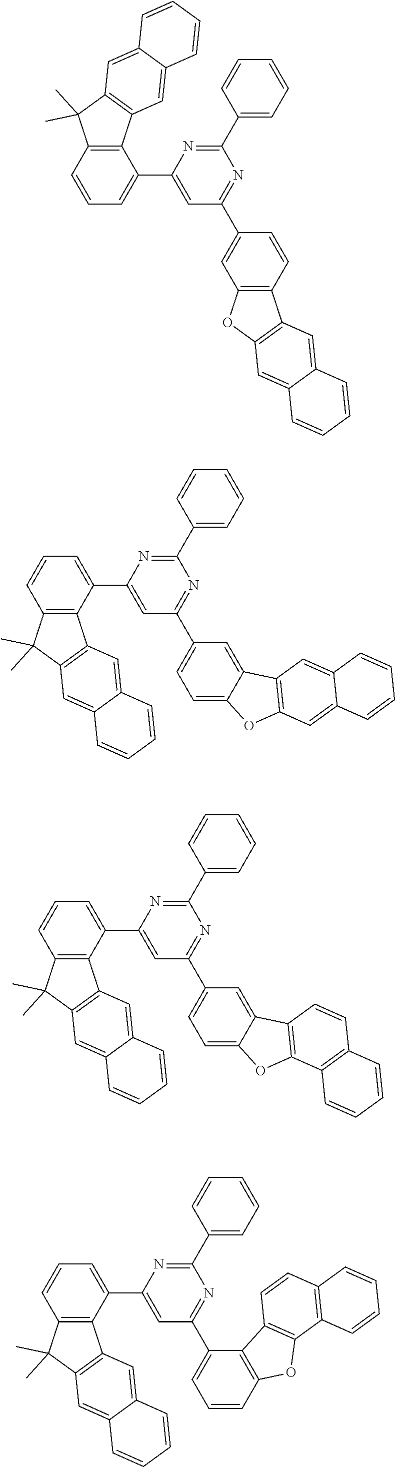 [SMILES: CC1(C)c2cc3ccccc3cc2-c2c(-c3cc(-c4ccc5c(c4)oc4cc6ccccc6cc45)nc(-c4ccccc4)n3)cccc21 ; CC1(C)c2cc3ccccc3cc2-c2c(-c3cc(-c4ccc5oc6cc7ccccc7cc6c5c4)nc(-c4ccccc4)n3)cccc21 ; CC1(C)c2cc3ccccc3cc2-c2c(-c3cc(-c4ccc5oc6c7ccccc7ccc6c5c4)nc(-c4ccccc4)n3)cccc21 ; CC1(C)c2cc3ccccc3cc2-c2c(-c3cc(-c4cccc5oc6c7ccccc7ccc6c45)nc(-c4ccccc4)n3)cccc21]

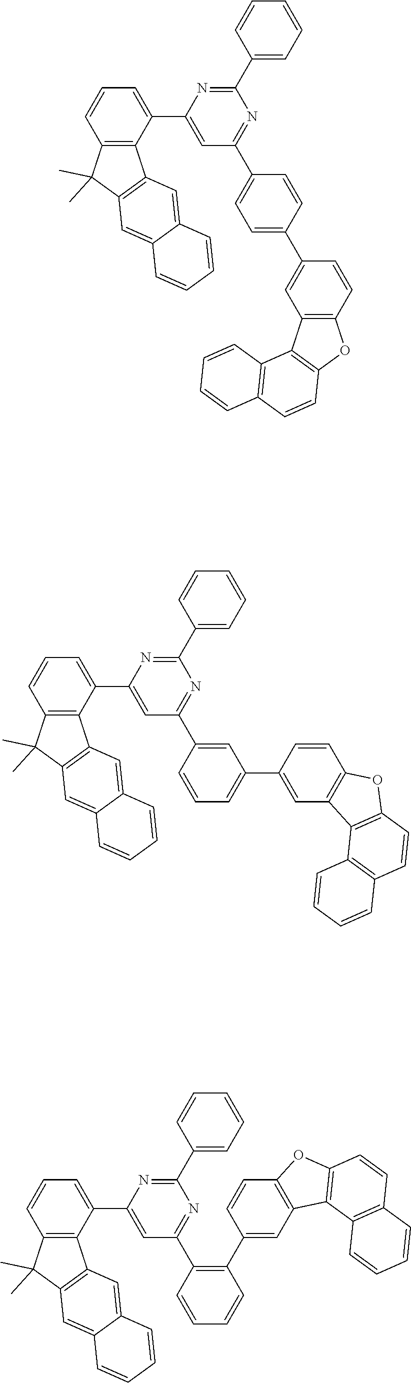 [SMILES: CC1(C)c2cc3ccccc3cc2-c2c(-c3cc(-c4ccc(-c5ccc6oc7ccc8ccccc8c7c6c5)cc4)nc(-c4ccccc4)n3)cccc21 ; CC1(C)c2cc3ccccc3cc2-c2c(-c3cc(-c4cccc(-c5ccc6oc7ccc8ccccc8c7c6c5)c4)nc(-c4ccccc4)n3)cccc21 ; CC1(C)c2cc3ccccc3cc2-c2c(-c3cc(-c4ccccc4-c4ccc5oc6ccc7ccccc7c6c5c4)nc(-c4ccccc4)n3)cccc21]

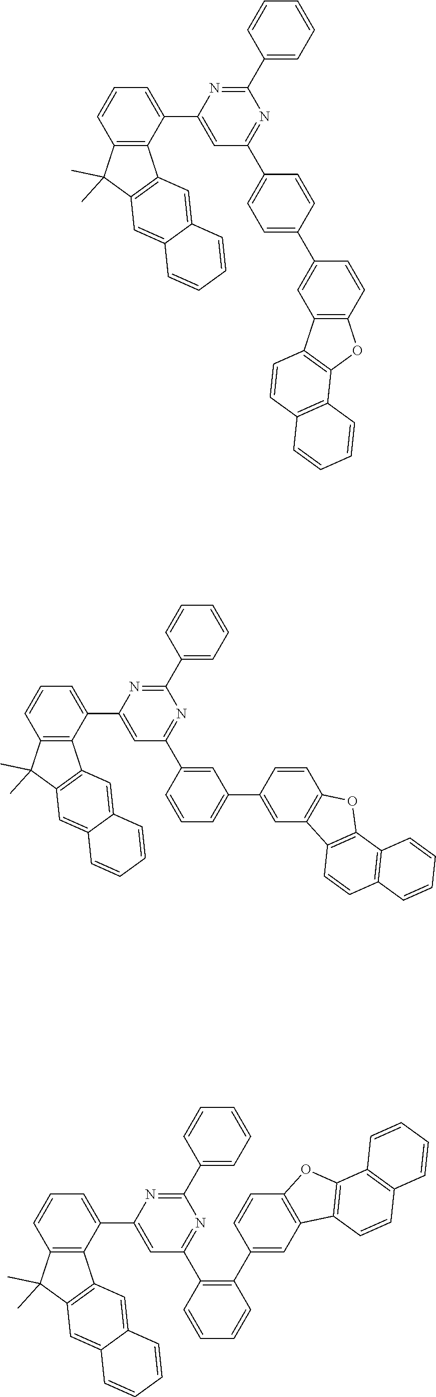 [SMILES: CC1(C)c2cc3ccccc3cc2-c2c(-c3cc(-c4ccc(-c5ccc6oc7c8ccccc8ccc7c6c5)cc4)nc(-c4ccccc4)n3)cccc21 ; CC1(C)c2cc3ccccc3cc2-c2c(-c3cc(-c4cccc(-c5ccc6oc7c8ccccc8ccc7c6c5)c4)nc(-c4ccccc4)n3)cccc21 ; CC1(C)c2cc3ccccc3cc2-c2c(-c3cc(-c4ccccc4-c4ccc5oc6c7ccccc7ccc6c5c4)nc(-c4ccccc4)n3)cccc21]

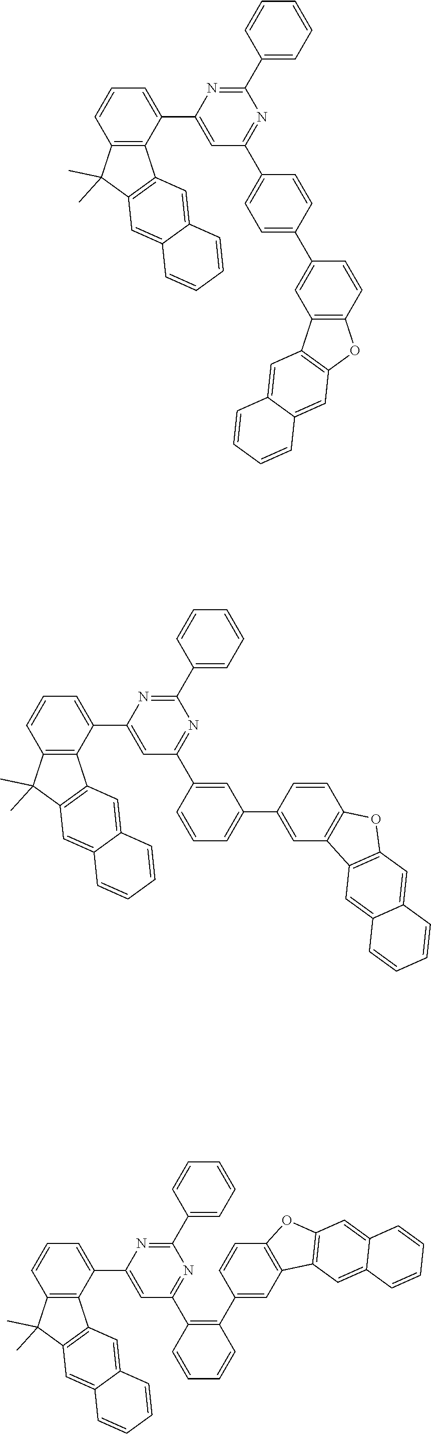 [SMILES: CC1(C)c2cc3ccccc3cc2-c2c(-c3cc(-c4ccc(-c5ccc6oc7cc8ccccc8cc7c6c5)cc4)nc(-c4ccccc4)n3)cccc21 ; CC1(C)c2cc3ccccc3cc2-c2c(-c3cc(-c4cccc(-c5ccc6oc7cc8ccccc8cc7c6c5)c4)nc(-c4ccccc4)n3)cccc21 ; CC1(C)c2cc3ccccc3cc2-c2c(-c3cc(-c4ccccc4-c4ccc5oc6cc7ccccc7cc6c5c4)nc(-c4ccccc4)n3)cccc21]

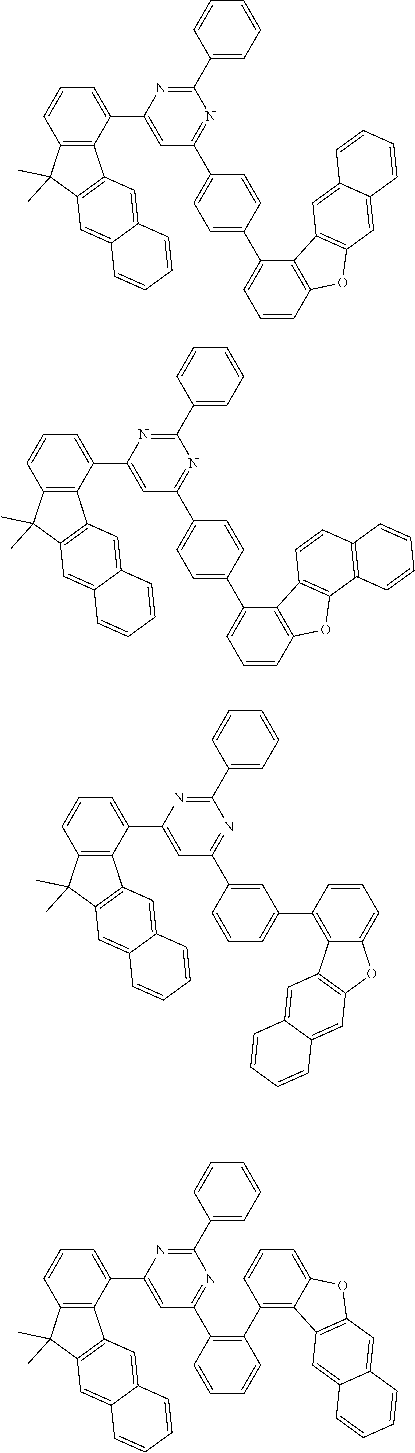

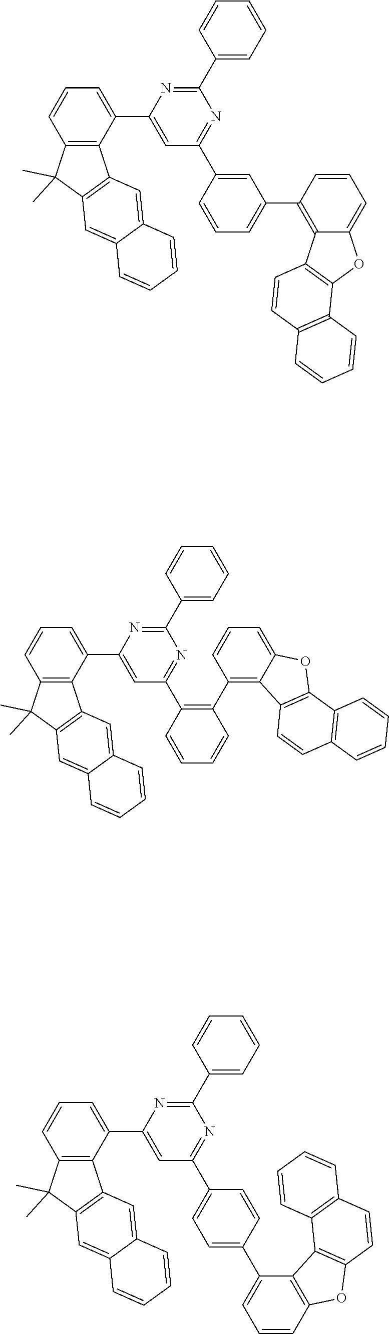 [SMILES: CC1(C)c2cc3ccccc3cc2-c2c(-c3cc(-c4cccc(-c5cccc6oc7c8ccccc8ccc7c56)c4)nc(-c4ccccc4)n3)cccc21 ; CC1(C)c2cc3ccccc3cc2-c2c(-c3cc(-c4ccccc4-c4cccc5oc6c7ccccc7ccc6c45)nc(-c4ccccc4)n3)cccc21 ; CC1(C)c2cc3ccccc3cc2-c2c(-c3cc(-c4ccc(-c5cccc6oc7ccc8ccccc8c7c56)cc4)nc(-c4ccccc4)n3)cccc21]

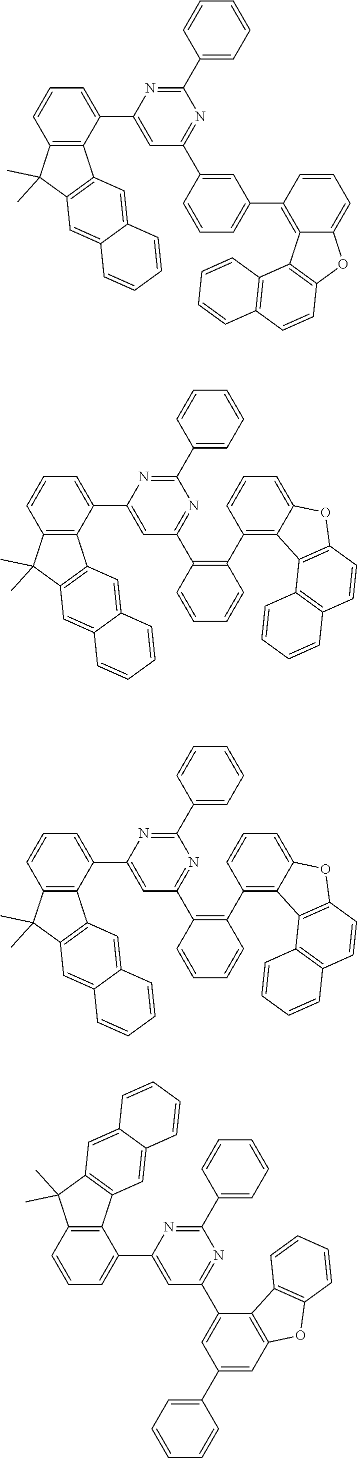 [SMILES: CC1(C)c2cc3ccccc3cc2-c2c(-c3cc(-c4cccc(-c5cccc6oc7ccc8ccccc8c7c56)c4)nc(-c4ccccc4)n3)cccc21 ; CC1(C)c2cc3ccccc3cc2-c2c(-c3cc(-c4ccccc4-c4cccc5oc6ccc7ccccc7c6c45)nc(-c4ccccc4)n3)cccc21 ; CC1(C)c2cc3ccccc3cc2-c2c(-c3cc(-c4ccccc4-c4cccc5oc6ccc7ccccc7c6c45)nc(-c4ccccc4)n3)cccc21 ; CC1(C)c2cc3ccccc3cc2-c2c(-c3cc(-c4cc(-c5ccccc5)cc5oc6ccccc6c45)nc(-c4ccccc4)n3)cccc21]

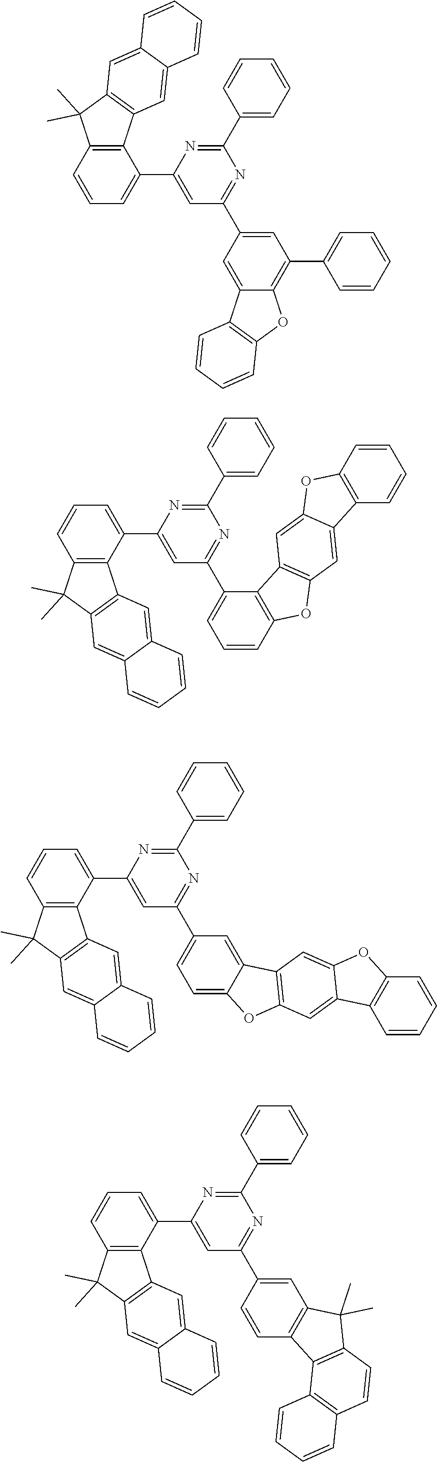

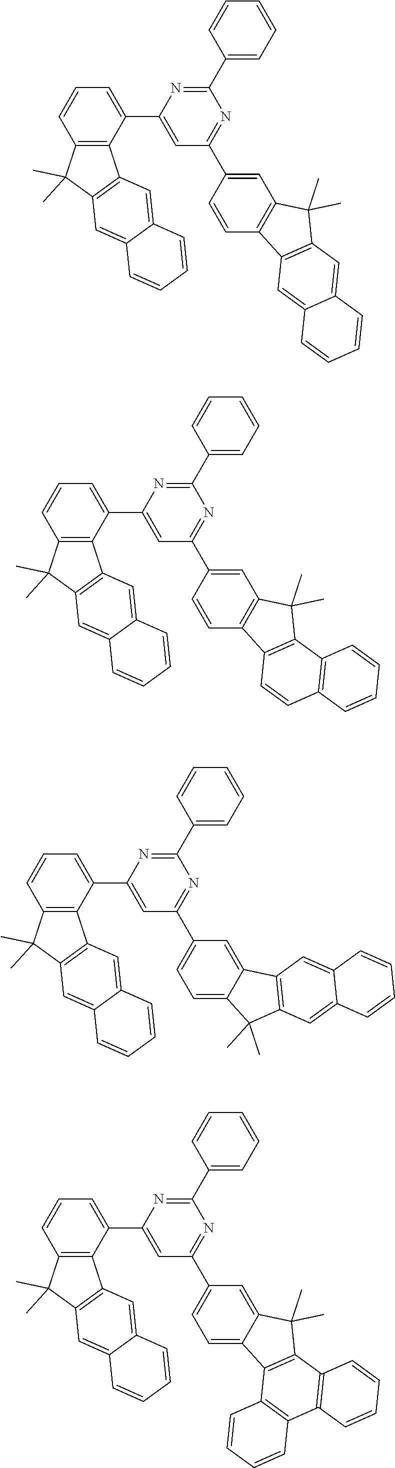 [SMILES: CC1(C)c2cc(-c3cc(-c4cccc5c4-c4cc6ccccc6cc4C5(C)C)nc(-c4ccccc4)n3)ccc2-c2cc3ccccc3cc21 ; CC1(C)c2cc3ccccc3cc2-c2c(-c3cc(-c4ccc5c(c4)C(C)(C)c4c-5ccc5ccccc45)nc(-c4ccccc4)n3)cccc21 ; CC1(C)c2ccc(-c3cc(-c4cccc5c4-c4cc6ccccc6cc4C5(C)C)nc(-c4ccccc4)n3)cc2-c2cc3ccccc3cc21 ; CC1(C)c2cc3ccccc3cc2-c2c(-c3cc(-c4ccc5c(c4)C(C)(C)c4c-5c5ccccc5c5ccccc45)nc(-c4ccccc4)n3)cccc21]

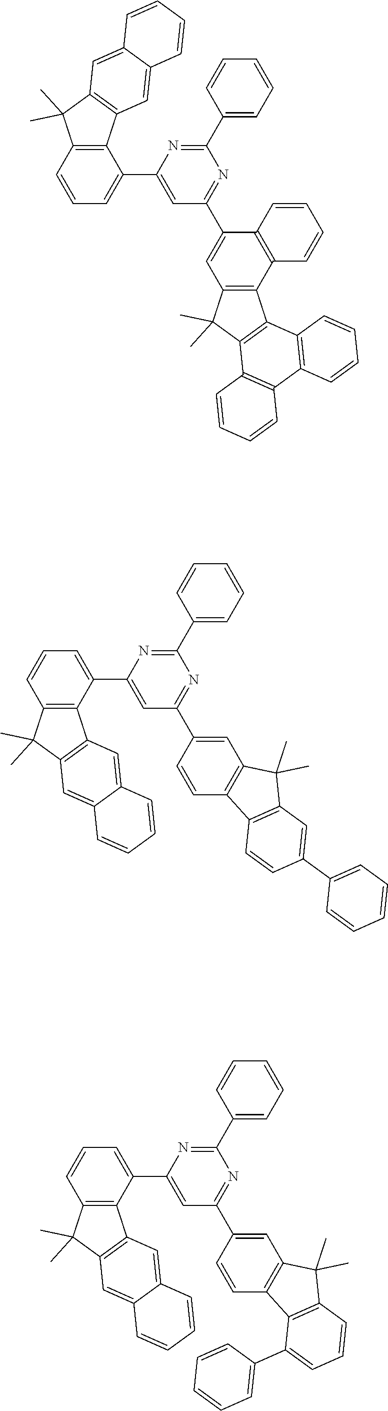

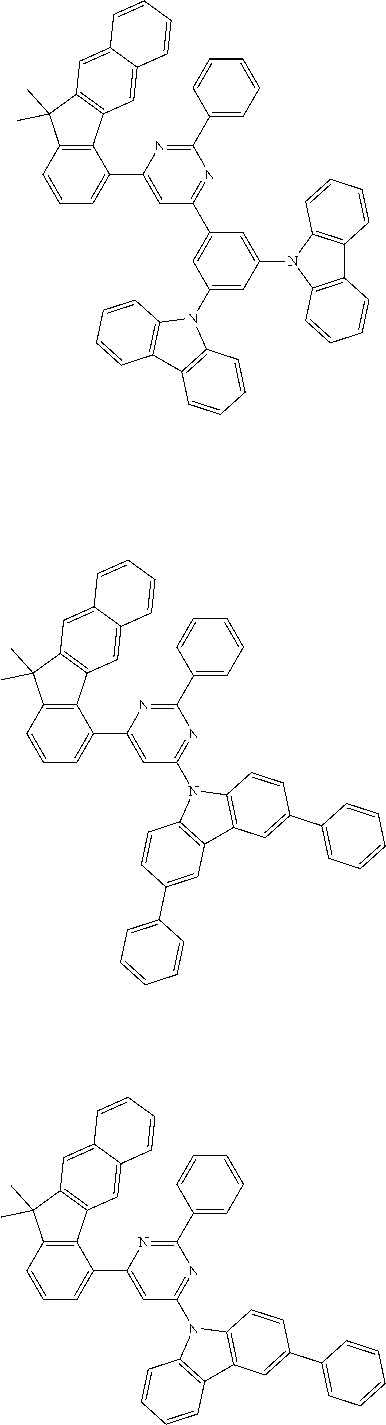 [SMILES: CC1(C)c2cc3ccccc3cc2-c2c(-c3cc(-c4cc(-n5c6ccccc6c6ccccc65)cc(-n5c6ccccc6c6ccccc65)c4)nc(-c4ccccc4)n3)cccc21 ; CC1(C)c2cc3ccccc3cc2-c2c(-c3cc(-n4c5ccc(-c6ccccc6)cc5c5cc(-c6ccccc6)ccc54)nc(-c4ccccc4)n3)cccc21 ; CC1(C)c2cc3ccccc3cc2-c2c(-c3cc(-n4c5ccccc5c5cc(-c6ccccc6)ccc54)nc(-c4ccccc4)n3)cccc21]

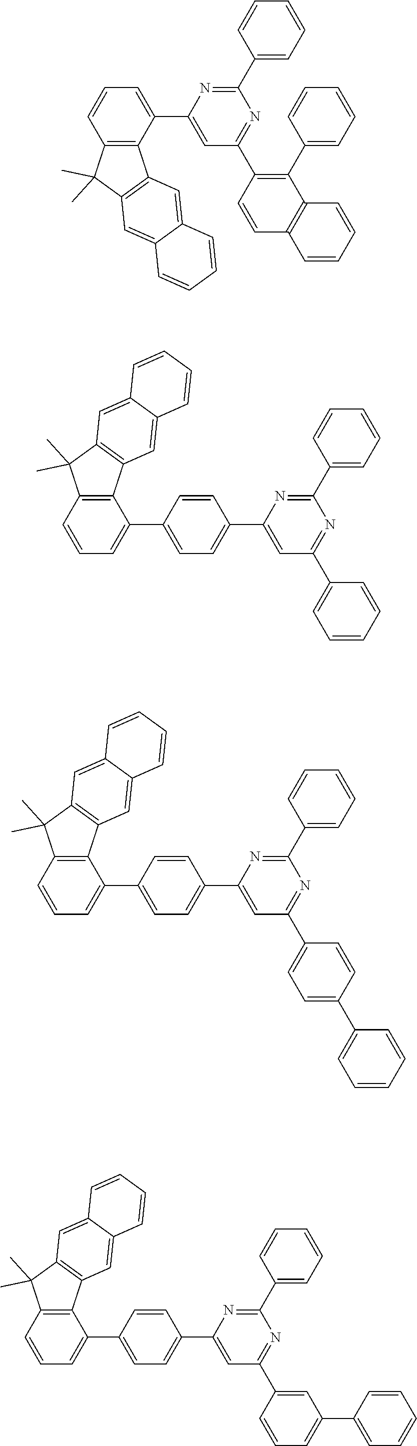 [SMILES: CC1(C)c2cc3ccccc3cc2-c2c(-c3cc(-c4ccc5ccccc5c4-c4ccccc4)nc(-c4ccccc4)n3)cccc21 ; CC1(C)c2cc3ccccc3cc2-c2c(-c3ccc(-c4cc(-c5ccccc5)nc(-c5ccccc5)n4)cc3)cccc21 ; CC1(C)c2cc3ccccc3cc2-c2c(-c3ccc(-c4cc(-c5ccc(-c6ccccc6)cc5)nc(-c5ccccc5)n4)cc3)cccc21 ; CC1(C)c2cc3ccccc3cc2-c2c(-c3ccc(-c4cc(-c5cccc(-c6ccccc6)c5)nc(-c5ccccc5)n4)cc3)cccc21]

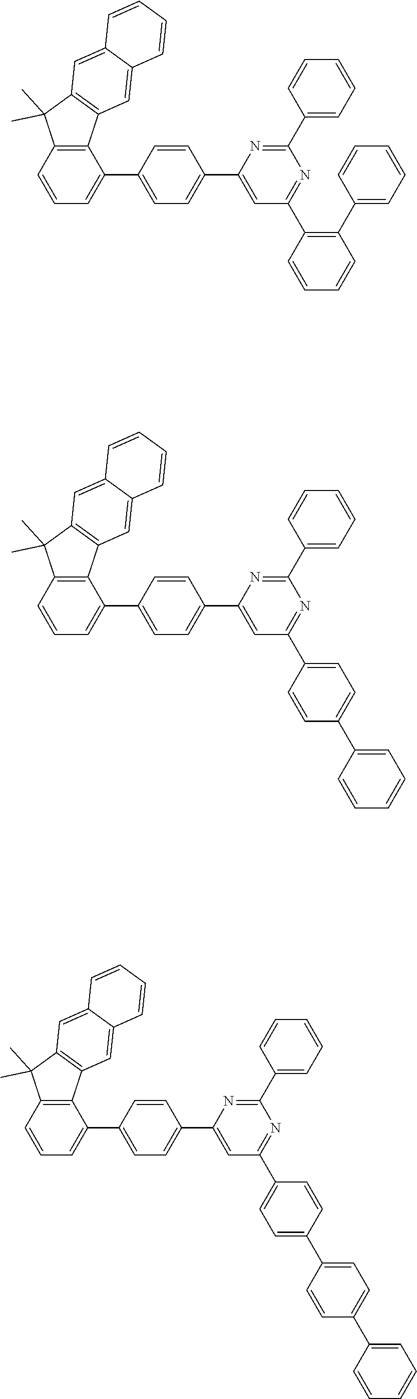 [SMILES: CC1(C)c2cc3ccccc3cc2-c2c(-c3ccc(-c4cc(-c5ccccc5-c5ccccc5)nc(-c5ccccc5)n4)cc3)cccc21 ; CC1(C)c2cc3ccccc3cc2-c2c(-c3ccc(-c4cc(-c5ccc(-c6ccccc6)cc5)nc(-c5ccccc5)n4)cc3)cccc21 ; CC1(C)c2cc3ccccc3cc2-c2c(-c3ccc(-c4cc(-c5ccc(-c6ccc(-c7ccccc7)cc6)cc5)nc(-c5ccccc5)n4)cc3)cccc21]

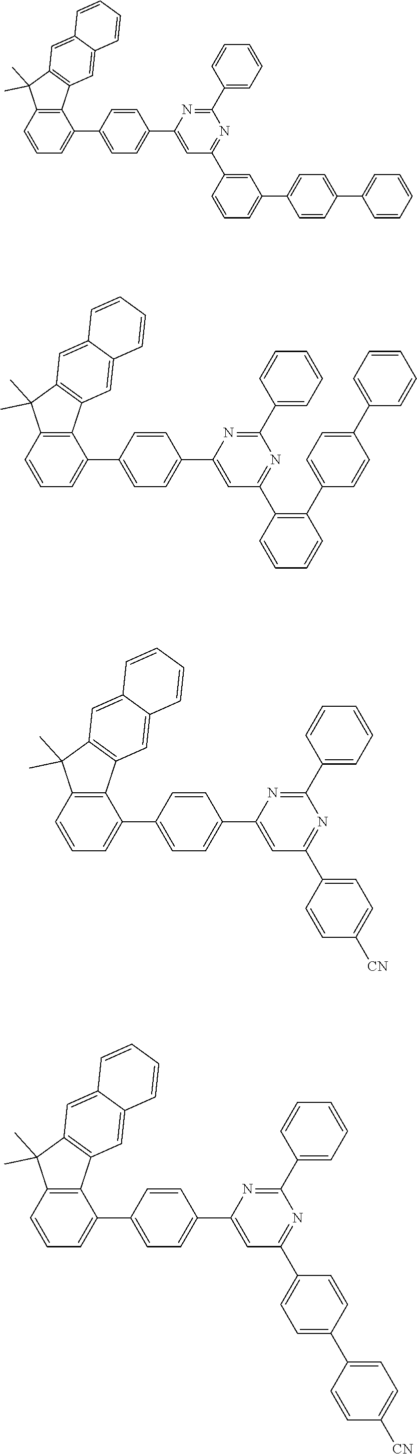 [SMILES: CC1(C)c2cc3ccccc3cc2-c2c(-c3ccc(-c4cc(-c5cccc(-c6ccc(-c7ccccc7)cc6)c5)nc(-c5ccccc5)n4)cc3)cccc21 ; CC1(C)c2cc3ccccc3cc2-c2c(-c3ccc(-c4cc(-c5ccccc5-c5ccc(-c6ccccc6)cc5)nc(-c5ccccc5)n4)cc3)cccc21 ; CC1(C)c2cc3ccccc3cc2-c2c(-c3ccc(-c4cc(-c5ccc(C#N)cc5)nc(-c5ccccc5)n4)cc3)cccc21 ; CC1(C)c2cc3ccccc3cc2-c2c(-c3ccc(-c4cc(-c5ccc(-c6ccc(C#N)cc6)cc5)nc(-c5ccccc5)n4)cc3)cccc21]

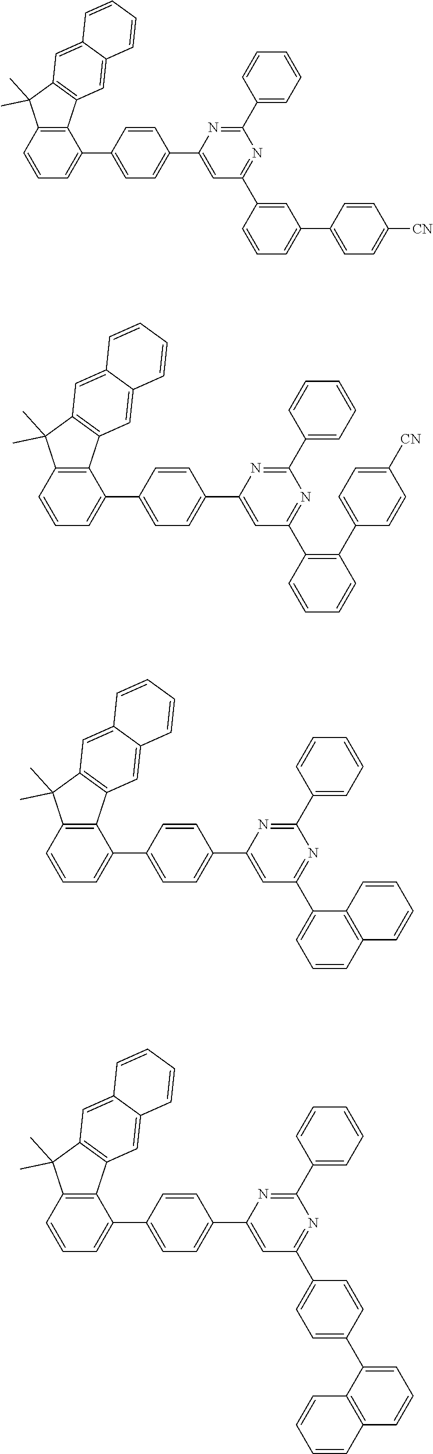 [SMILES: CC1(C)c2cc3ccccc3cc2-c2c(-c3ccc(-c4cc(-c5cccc(-c6ccc(C#N)cc6)c5)nc(-c5ccccc5)n4)cc3)cccc21 ; CC1(C)c2cc3ccccc3cc2-c2c(-c3ccc(-c4cc(-c5ccccc5-c5ccc(C#N)cc5)nc(-c5ccccc5)n4)cc3)cccc21 ; CC1(C)c2cc3ccccc3cc2-c2c(-c3ccc(-c4cc(-c5cccc6ccccc56)nc(-c5ccccc5)n4)cc3)cccc21 ; CC1(C)c2cc3ccccc3cc2-c2c(-c3ccc(-c4cc(-c5ccc(-c6cccc7ccccc67)cc5)nc(-c5ccccc5)n4)cc3)cccc21]

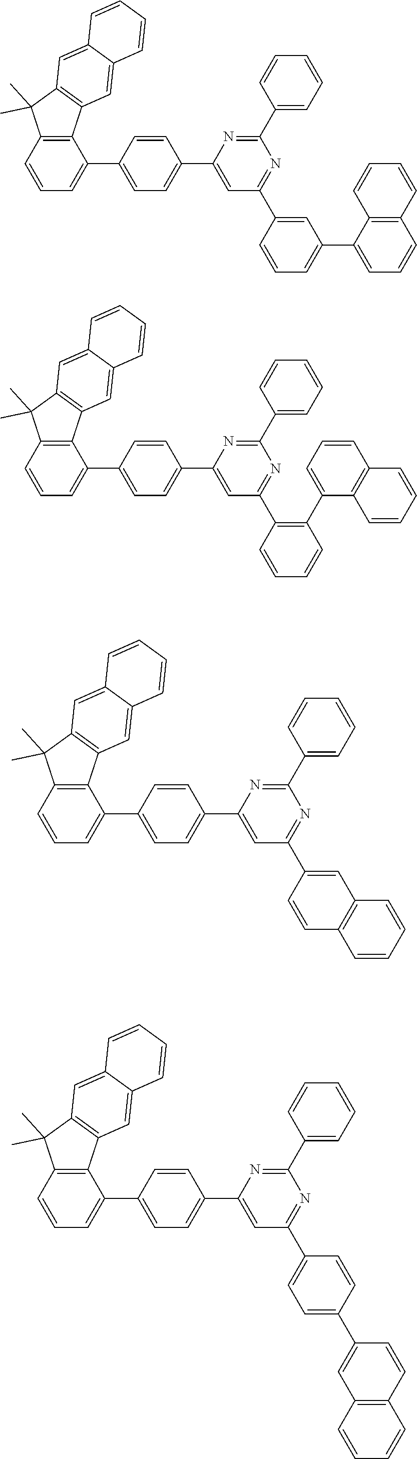 [SMILES: CC1(C)c2cc3ccccc3cc2-c2c(-c3ccc(-c4cc(-c5cccc(-c6cccc7ccccc67)c5)nc(-c5ccccc5)n4)cc3)cccc21 ; CC1(C)c2cc3ccccc3cc2-c2c(-c3ccc(-c4cc(-c5ccccc5-c5cccc6ccccc56)nc(-c5ccccc5)n4)cc3)cccc21 ; CC1(C)c2cc3ccccc3cc2-c2c(-c3ccc(-c4cc(-c5ccc6ccccc6c5)nc(-c5ccccc5)n4)cc3)cccc21 ; CC1(C)c2cc3ccccc3cc2-c2c(-c3ccc(-c4cc(-c5ccc(-c6ccc7ccccc7c6)cc5)nc(-c5ccccc5)n4)cc3)cccc21]

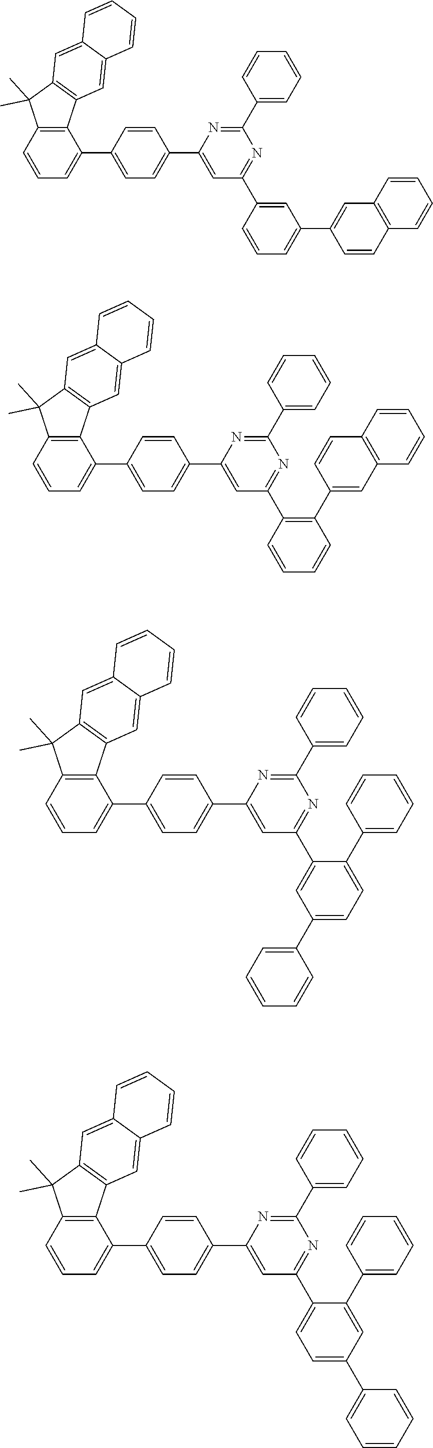 [SMILES: CC1(C)c2cc3ccccc3cc2-c2c(-c3ccc(-c4cc(-c5cccc(-c6ccc7ccccc7c6)c5)nc(-c5ccccc5)n4)cc3)cccc21 ; CC1(C)c2cc3ccccc3cc2-c2c(-c3ccc(-c4cc(-c5ccccc5-c5ccc6ccccc6c5)nc(-c5ccccc5)n4)cc3)cccc21 ; CC1(C)c2cc3ccccc3cc2-c2c(-c3ccc(-c4cc(-c5cc(-c6ccccc6)ccc5-c5ccccc5)nc(-c5ccccc5)n4)cc3)cccc21 ; CC1(C)c2cc3ccccc3cc2-c2c(-c3ccc(-c4cc(-c5ccc(-c6ccccc6)cc5-c5ccccc5)nc(-c5ccccc5)n4)cc3)cccc21]

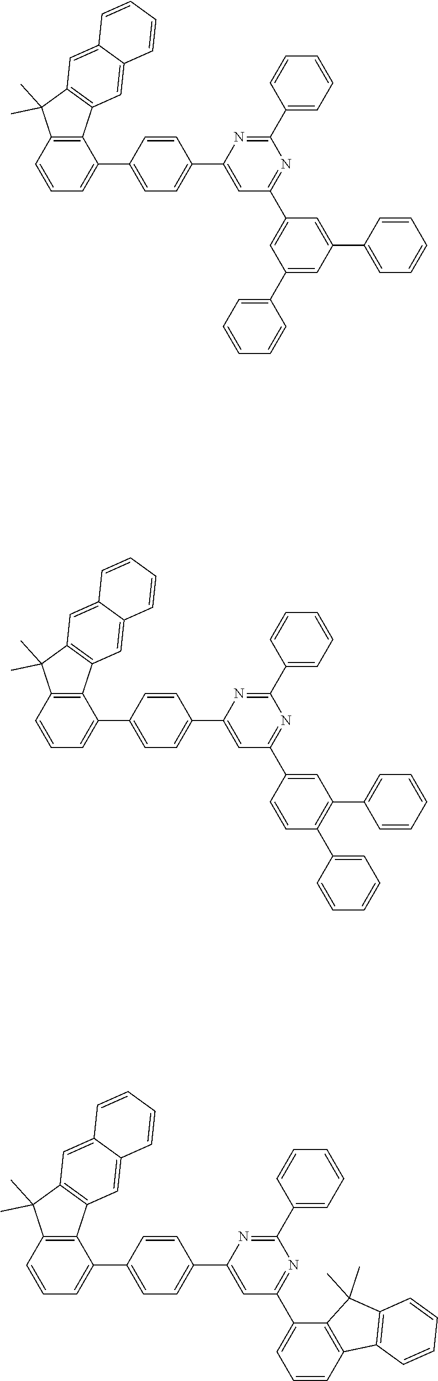 [SMILES: CC1(C)c2cc3ccccc3cc2-c2c(-c3ccc(-c4cc(-c5cc(-c6ccccc6)cc(-c6ccccc6)c5)nc(-c5ccccc5)n4)cc3)cccc21 ; CC1(C)c2cc3ccccc3cc2-c2c(-c3ccc(-c4cc(-c5ccc(-c6ccccc6)c(-c6ccccc6)c5)nc(-c5ccccc5)n4)cc3)cccc21 ; CC1(C)c2cc3ccccc3cc2-c2c(-c3ccc(-c4cc(-c5cccc6c5C(C)(C)c5ccccc5-6)nc(-c5ccccc5)n4)cc3)cccc21]

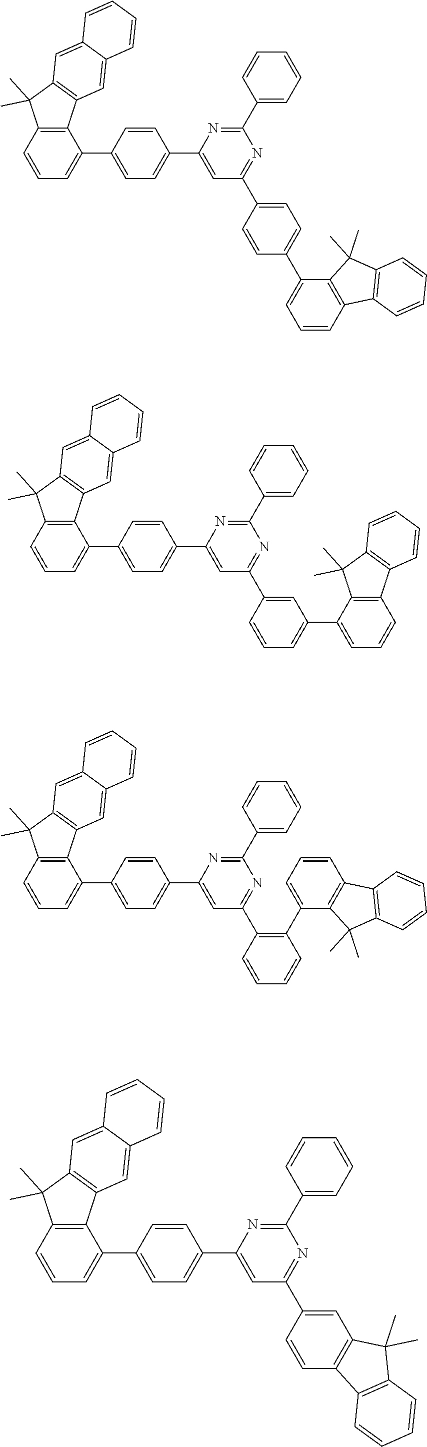

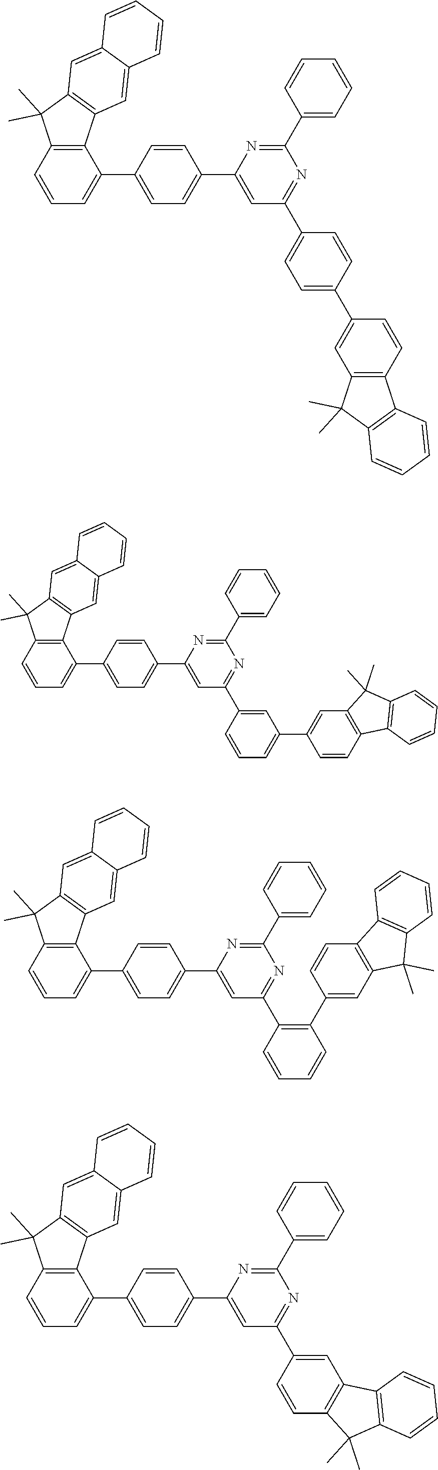

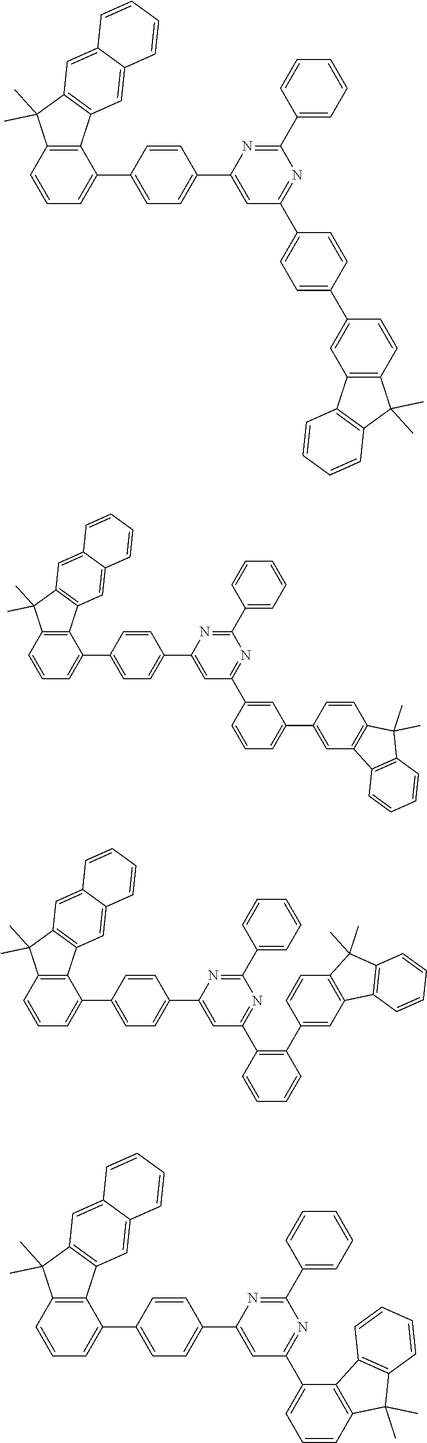

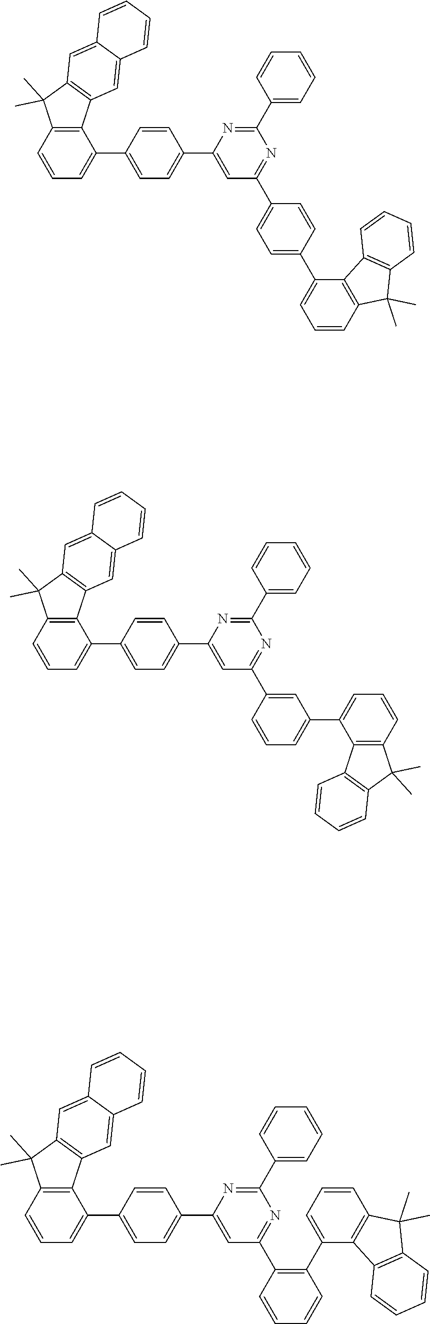

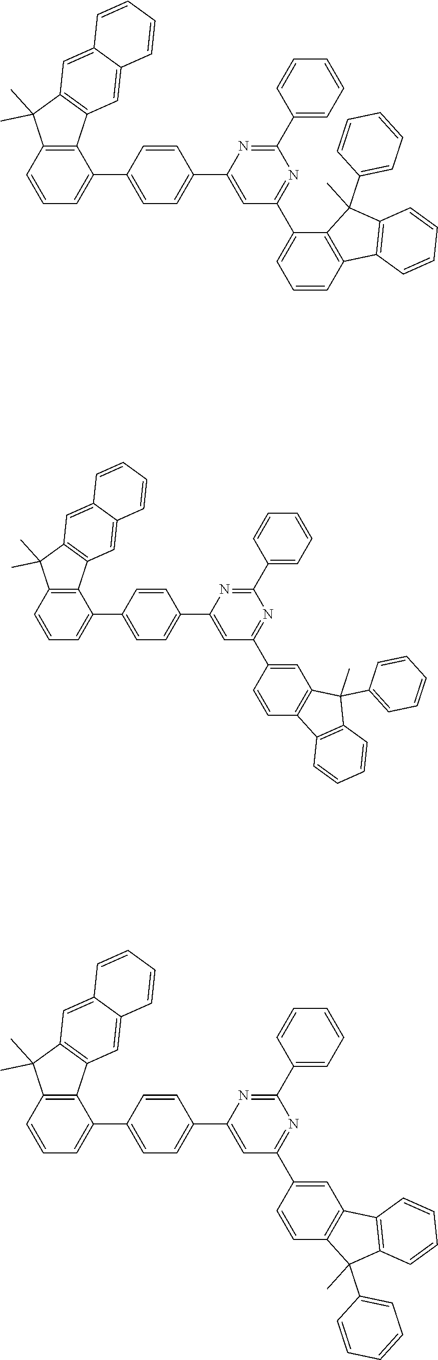 [SMILES: CC1(C)c2cc3ccccc3cc2-c2c(-c3ccc(-c4cc(-c5cccc6c5C(C)(c5ccccc5)c5ccccc5-6)nc(-c5ccccc5)n4)cc3)cccc21 ; CC1(C)c2cc3ccccc3cc2-c2c(-c3ccc(-c4cc(-c5ccc6c(c5)C(C)(c5ccccc5)c5ccccc5-6)nc(-c5ccccc5)n4)cc3)cccc21 ; CC1(C)c2cc3ccccc3cc2-c2c(-c3ccc(-c4cc(-c5ccc6c(c5)-c5ccccc5C6(C)c5ccccc5)nc(-c5ccccc5)n4)cc3)cccc21]

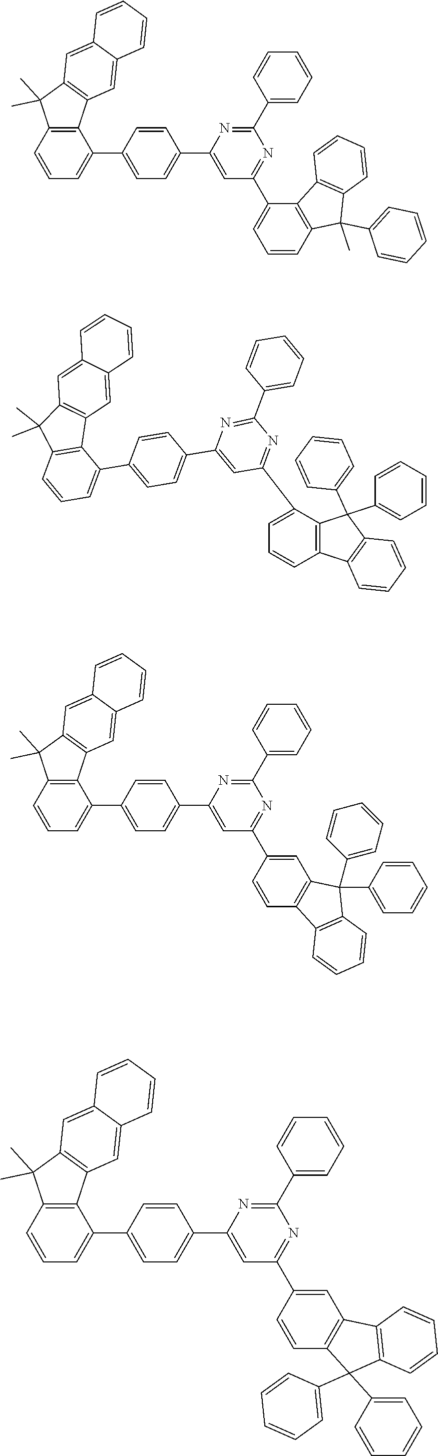 [SMILES: CC1(C)c2cc3ccccc3cc2-c2c(-c3ccc(-c4cc(-c5cccc6c5-c5ccccc5C6(C)c5ccccc5)nc(-c5ccccc5)n4)cc3)cccc21 ; CC1(C)c2cc3ccccc3cc2-c2c(-c3ccc(-c4cc(-c5cccc6c5C(c5ccccc5)(c5ccccc5)c5ccccc5-6)nc(-c5ccccc5)n4)cc3)cccc21 ; CC1(C)c2cc3ccccc3cc2-c2c(-c3ccc(-c4cc(-c5ccc6c(c5)C(c5ccccc5)(c5ccccc5)c5ccccc5-6)nc(-c5ccccc5)n4)cc3)cccc21 ; CC1(C)c2cc3ccccc3cc2-c2c(-c3ccc(-c4cc(-c5ccc6c(c5)-c5ccccc5C6(c5ccccc5)c5ccccc5)nc(-c5ccccc5)n4)cc3)cccc21]

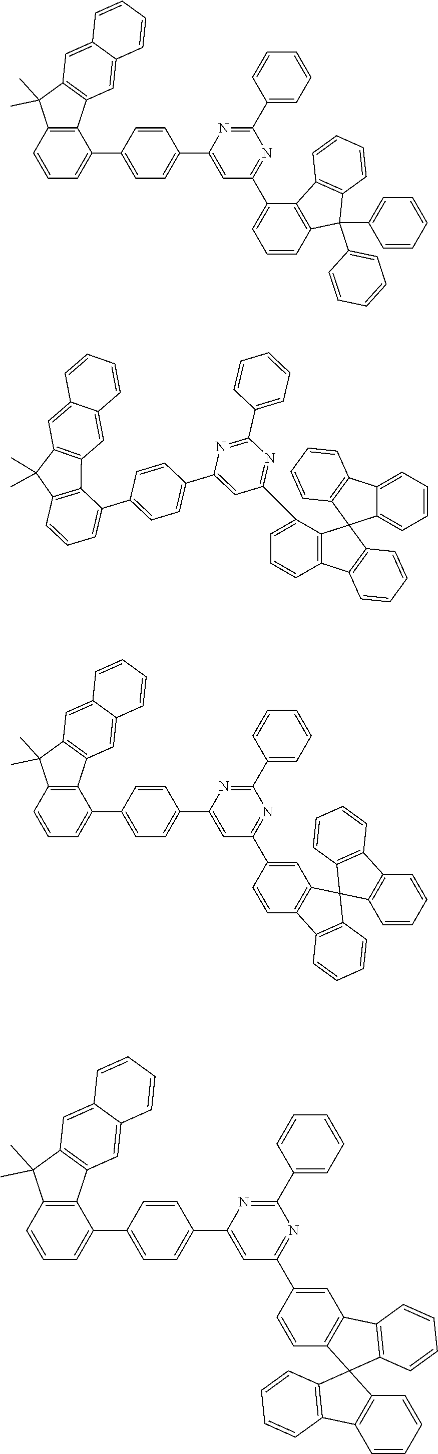 [SMILES: CC1(C)c2cc3ccccc3cc2-c2c(-c3ccc(-c4cc(-c5cccc6c5-c5ccccc5C6(c5ccccc5)c5ccccc5)nc(-c5ccccc5)n4)cc3)cccc21 ; CC1(C)c2cc3ccccc3cc2-c2c(-c3ccc(-c4cc(-c5cccc6c5C5(c7ccccc7-c7ccccc75)c5ccccc5-6)nc(-c5ccccc5)n4)cc3)cccc21 ; CC1(C)c2cc3ccccc3cc2-c2c(-c3ccc(-c4cc(-c5ccc6c(c5)C5(c7ccccc7-c7ccccc75)c5ccccc5-6)nc(-c5ccccc5)n4)cc3)cccc21 ; CC1(C)c2cc3ccccc3cc2-c2c(-c3ccc(-c4cc(-c5ccc6c(c5)-c5ccccc5C65c6ccccc6-c6ccccc65)nc(-c5ccccc5)n4)cc3)cccc21]

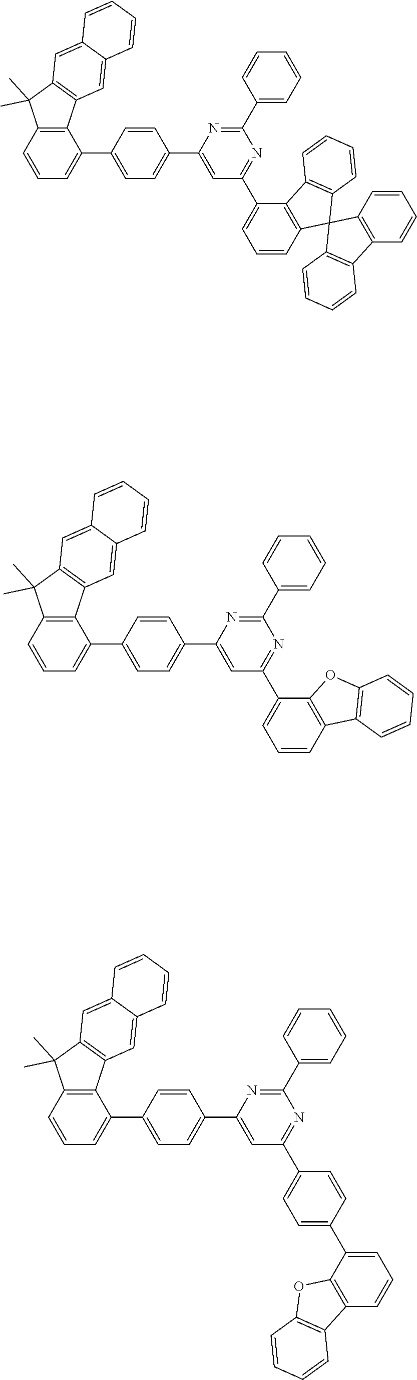 [SMILES: CC1(C)c2cc3ccccc3cc2-c2c(-c3ccc(-c4cc(-c5cccc6c5-c5ccccc5C65c6ccccc6-c6ccccc65)nc(-c5ccccc5)n4)cc3)cccc21 ; CC1(C)c2cc3ccccc3cc2-c2c(-c3ccc(-c4cc(-c5cccc6c5oc5ccccc56)nc(-c5ccccc5)n4)cc3)cccc21 ; CC1(C)c2cc3ccccc3cc2-c2c(-c3ccc(-c4cc(-c5ccc(-c6cccc7c6oc6ccccc67)cc5)nc(-c5ccccc5)n4)cc3)cccc21]

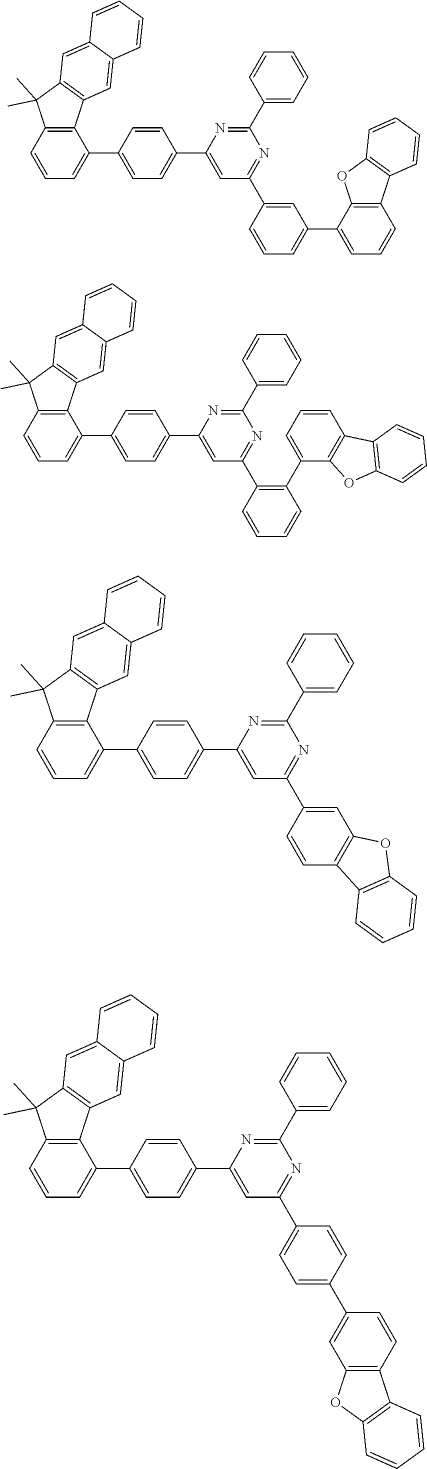 [SMILES: CC1(C)c2cc3ccccc3cc2-c2c(-c3ccc(-c4cc(-c5cccc(-c6cccc7c6oc6ccccc67)c5)nc(-c5ccccc5)n4)cc3)cccc21 ; CC1(C)c2cc3ccccc3cc2-c2c(-c3ccc(-c4cc(-c5ccccc5-c5cccc6c5oc5ccccc56)nc(-c5ccccc5)n4)cc3)cccc21 ; CC1(C)c2cc3ccccc3cc2-c2c(-c3ccc(-c4cc(-c5ccc6c(c5)oc5ccccc56)nc(-c5ccccc5)n4)cc3)cccc21 ; CC1(C)c2cc3ccccc3cc2-c2c(-c3ccc(-c4cc(-c5ccc(-c6ccc7c(c6)oc6ccccc67)cc5)nc(-c5ccccc5)n4)cc3)cccc21]

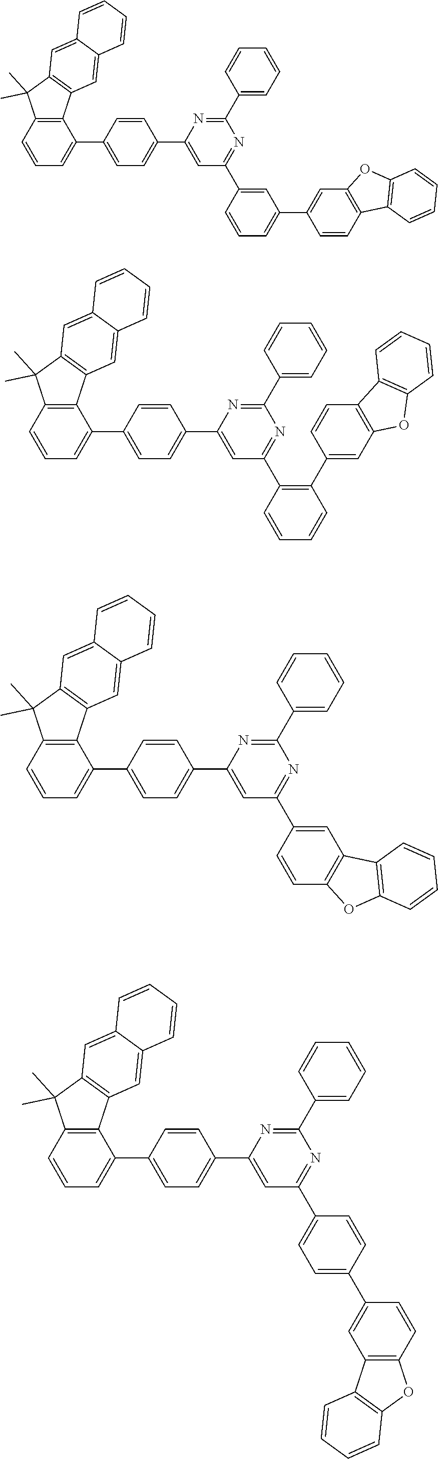 [SMILES: CC1(C)c2cc3ccccc3cc2-c2c(-c3ccc(-c4cc(-c5cccc(-c6ccc7c(c6)oc6ccccc67)c5)nc(-c5ccccc5)n4)cc3)cccc21 ; CC1(C)c2cc3ccccc3cc2-c2c(-c3ccc(-c4cc(-c5ccccc5-c5ccc6c(c5)oc5ccccc56)nc(-c5ccccc5)n4)cc3)cccc21 ; CC1(C)c2cc3ccccc3cc2-c2c(-c3ccc(-c4cc(-c5ccc6oc7ccccc7c6c5)nc(-c5ccccc5)n4)cc3)cccc21 ; CC1(C)c2cc3ccccc3cc2-c2c(-c3ccc(-c4cc(-c5ccc(-c6ccc7oc8ccccc8c7c6)cc5)nc(-c5ccccc5)n4)cc3)cccc21]

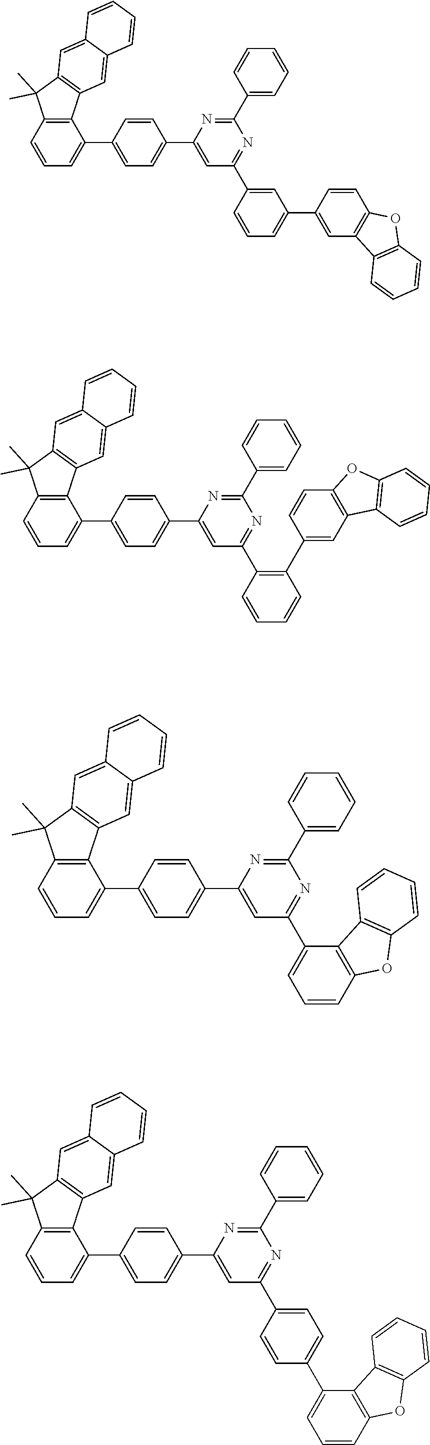 [SMILES: CC1(C)c2cc3ccccc3cc2-c2c(-c3ccc(-c4cc(-c5cccc(-c6ccc7oc8ccccc8c7c6)c5)nc(-c5ccccc5)n4)cc3)cccc21 ; CC1(C)c2cc3ccccc3cc2-c2c(-c3ccc(-c4cc(-c5ccccc5-c5ccc6oc7ccccc7c6c5)nc(-c5ccccc5)n4)cc3)cccc21 ; CC1(C)c2cc3ccccc3cc2-c2c(-c3ccc(-c4cc(-c5cccc6oc7ccccc7c56)nc(-c5ccccc5)n4)cc3)cccc21 ; CC1(C)c2cc3ccccc3cc2-c2c(-c3ccc(-c4cc(-c5ccc(-c6cccc7oc8ccccc8c67)cc5)nc(-c5ccccc5)n4)cc3)cccc21]

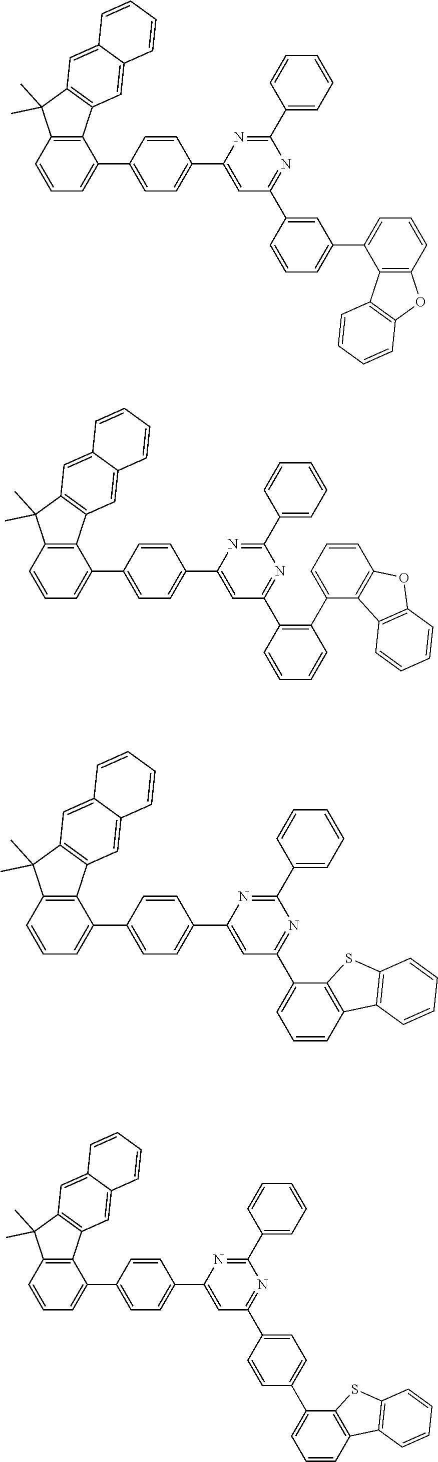 [SMILES: CC1(C)c2cc3ccccc3cc2-c2c(-c3ccc(-c4cc(-c5cccc(-c6cccc7oc8ccccc8c67)c5)nc(-c5ccccc5)n4)cc3)cccc21 ; CC1(C)c2cc3ccccc3cc2-c2c(-c3ccc(-c4cc(-c5ccccc5-c5cccc6oc7ccccc7c56)nc(-c5ccccc5)n4)cc3)cccc21 ; CC1(C)c2cc3ccccc3cc2-c2c(-c3ccc(-c4cc(-c5cccc6c5sc5ccccc56)nc(-c5ccccc5)n4)cc3)cccc21 ; CC1(C)c2cc3ccccc3cc2-c2c(-c3ccc(-c4cc(-c5ccc(-c6cccc7c6sc6ccccc67)cc5)nc(-c5ccccc5)n4)cc3)cccc21]

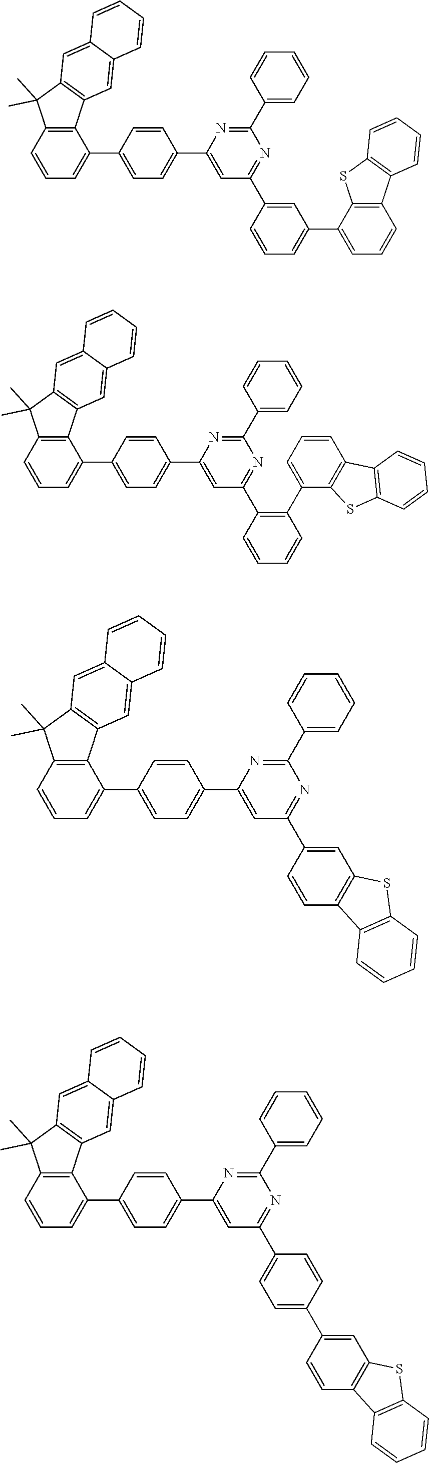 [SMILES: CC1(C)c2cc3ccccc3cc2-c2c(-c3ccc(-c4cc(-c5cccc(-c6cccc7c6sc6ccccc67)c5)nc(-c5ccccc5)n4)cc3)cccc21 ; CC1(C)c2cc3ccccc3cc2-c2c(-c3ccc(-c4cc(-c5ccccc5-c5cccc6c5sc5ccccc56)nc(-c5ccccc5)n4)cc3)cccc21 ; CC1(C)c2cc3ccccc3cc2-c2c(-c3ccc(-c4cc(-c5ccc6c(c5)sc5ccccc56)nc(-c5ccccc5)n4)cc3)cccc21 ; CC1(C)c2cc3ccccc3cc2-c2c(-c3ccc(-c4cc(-c5ccc(-c6ccc7c(c6)sc6ccccc67)cc5)nc(-c5ccccc5)n4)cc3)cccc21]

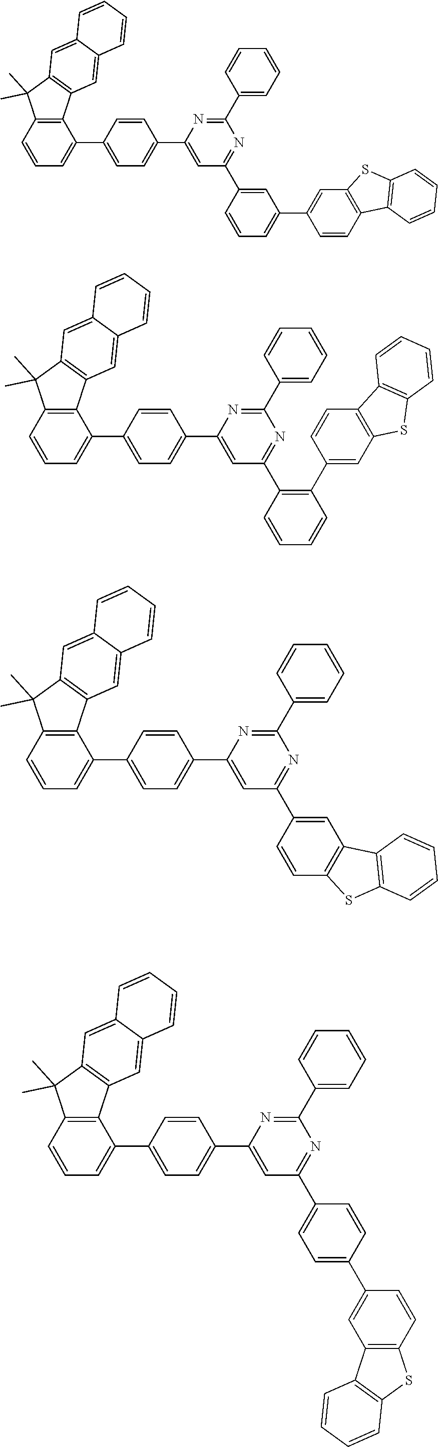 [SMILES: CC1(C)c2cc3ccccc3cc2-c2c(-c3ccc(-c4cc(-c5cccc(-c6ccc7c(c6)sc6ccccc67)c5)nc(-c5ccccc5)n4)cc3)cccc21 ; CC1(C)c2cc3ccccc3cc2-c2c(-c3ccc(-c4cc(-c5ccccc5-c5ccc6c(c5)sc5ccccc56)nc(-c5ccccc5)n4)cc3)cccc21 ; CC1(C)c2cc3ccccc3cc2-c2c(-c3ccc(-c4cc(-c5ccc6sc7ccccc7c6c5)nc(-c5ccccc5)n4)cc3)cccc21 ; CC1(C)c2cc3ccccc3cc2-c2c(-c3ccc(-c4cc(-c5ccc(-c6ccc7sc8ccccc8c7c6)cc5)nc(-c5ccccc5)n4)cc3)cccc21]

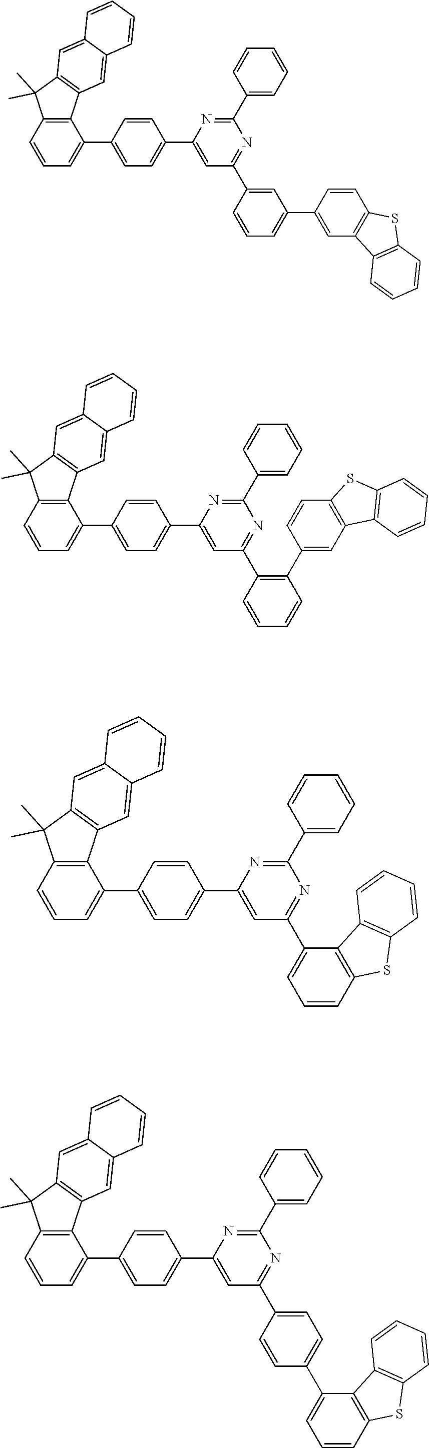 [SMILES: CC1(C)c2cc3ccccc3cc2-c2c(-c3ccc(-c4cc(-c5cccc(-c6ccc7sc8ccccc8c7c6)c5)nc(-c5ccccc5)n4)cc3)cccc21 ; CC1(C)c2cc3ccccc3cc2-c2c(-c3ccc(-c4cc(-c5ccccc5-c5ccc6sc7ccccc7c6c5)nc(-c5ccccc5)n4)cc3)cccc21 ; CC1(C)c2cc3ccccc3cc2-c2c(-c3ccc(-c4cc(-c5cccc6sc7ccccc7c56)nc(-c5ccccc5)n4)cc3)cccc21 ; CC1(C)c2cc3ccccc3cc2-c2c(-c3ccc(-c4cc(-c5ccc(-c6cccc7sc8ccccc8c67)cc5)nc(-c5ccccc5)n4)cc3)cccc21]

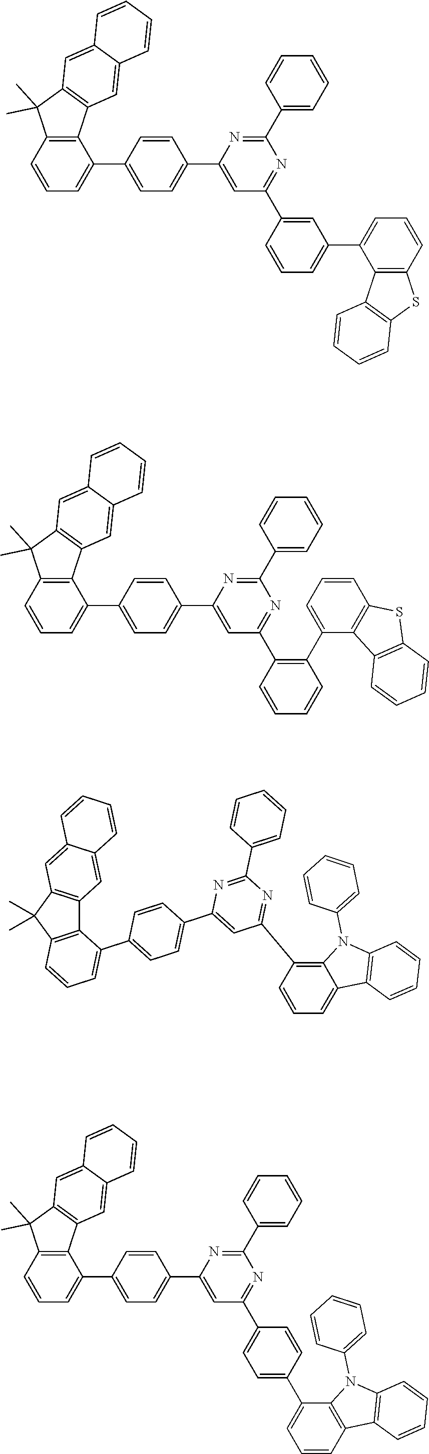 [SMILES: CC1(C)c2cc3ccccc3cc2-c2c(-c3ccc(-c4cc(-c5cccc(-c6cccc7sc8ccccc8c67)c5)nc(-c5ccccc5)n4)cc3)cccc21 ; CC1(C)c2cc3ccccc3cc2-c2c(-c3ccc(-c4cc(-c5ccccc5-c5cccc6sc7ccccc7c56)nc(-c5ccccc5)n4)cc3)cccc21 ; CC1(C)c2cc3ccccc3cc2-c2c(-c3ccc(-c4cc(-c5cccc6c7ccccc7n(-c7ccccc7)c56)nc(-c5ccccc5)n4)cc3)cccc21 ; CC1(C)c2cc3ccccc3cc2-c2c(-c3ccc(-c4cc(-c5ccc(-c6cccc7c8ccccc8n(-c8ccccc8)c67)cc5)nc(-c5ccccc5)n4)cc3)cccc21]

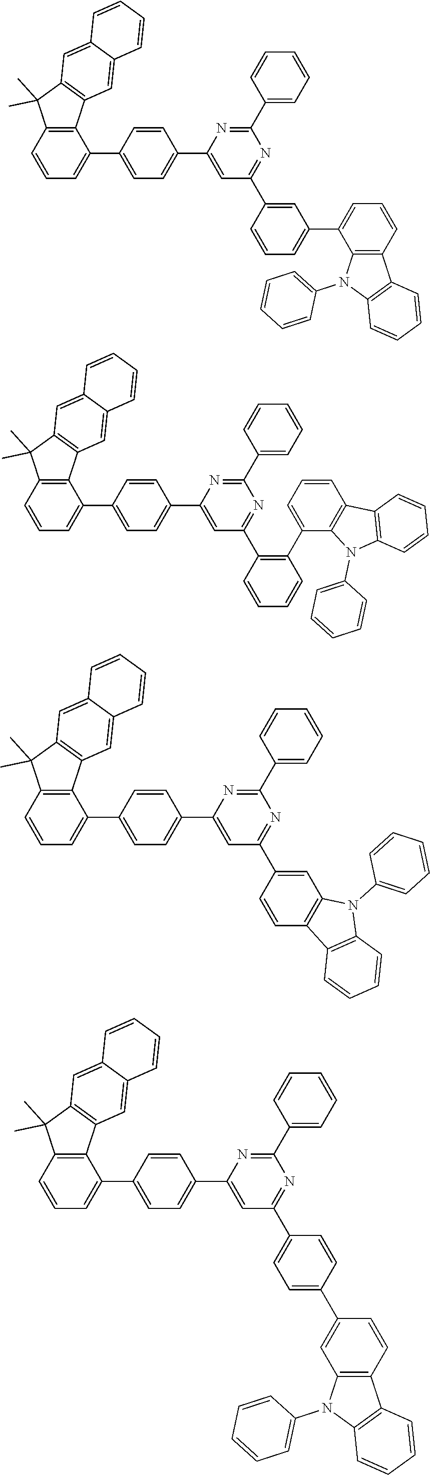 [SMILES: CC1(C)c2cc3ccccc3cc2-c2c(-c3ccc(-c4cc(-c5cccc(-c6cccc7c8ccccc8n(-c8ccccc8)c67)c5)nc(-c5ccccc5)n4)cc3)cccc21 ; CC1(C)c2cc3ccccc3cc2-c2c(-c3ccc(-c4cc(-c5ccccc5-c5cccc6c7ccccc7n(-c7ccccc7)c56)nc(-c5ccccc5)n4)cc3)cccc21 ; CC1(C)c2cc3ccccc3cc2-c2c(-c3ccc(-c4cc(-c5ccc6c7ccccc7n(-c7ccccc7)c6c5)nc(-c5ccccc5)n4)cc3)cccc21 ; CC1(C)c2cc3ccccc3cc2-c2c(-c3ccc(-c4cc(-c5ccc(-c6ccc7c8ccccc8n(-c8ccccc8)c7c6)cc5)nc(-c5ccccc5)n4)cc3)cccc21]

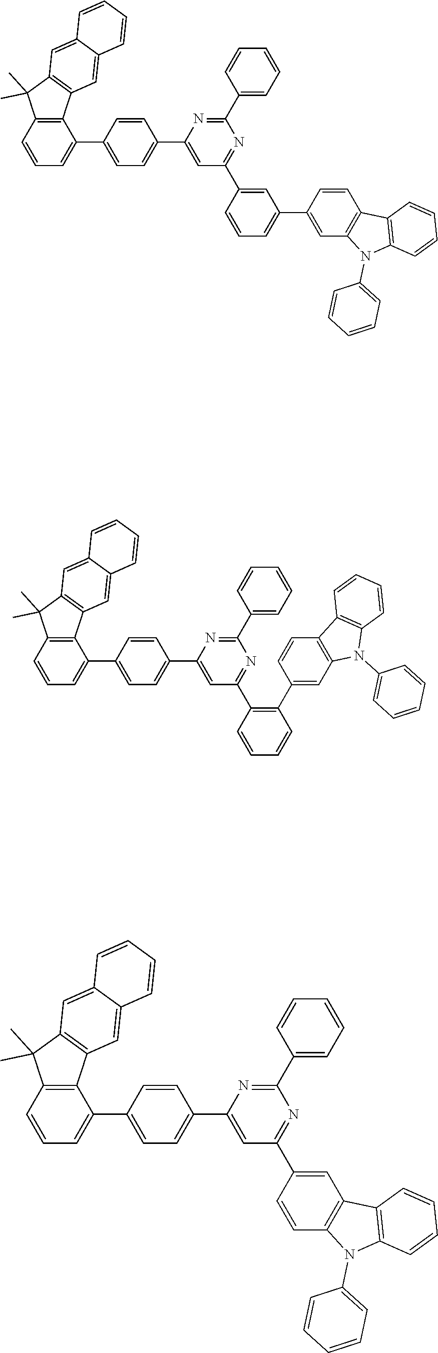 [SMILES: CC1(C)c2cc3ccccc3cc2-c2c(-c3ccc(-c4cc(-c5cccc(-c6ccc7c8ccccc8n(-c8ccccc8)c7c6)c5)nc(-c5ccccc5)n4)cc3)cccc21 ; CC1(C)c2cc3ccccc3cc2-c2c(-c3ccc(-c4cc(-c5ccccc5-c5ccc6c7ccccc7n(-c7ccccc7)c6c5)nc(-c5ccccc5)n4)cc3)cccc21 ; CC1(C)c2cc3ccccc3cc2-c2c(-c3ccc(-c4cc(-c5ccc6c(c5)c5ccccc5n6-c5ccccc5)nc(-c5ccccc5)n4)cc3)cccc21]

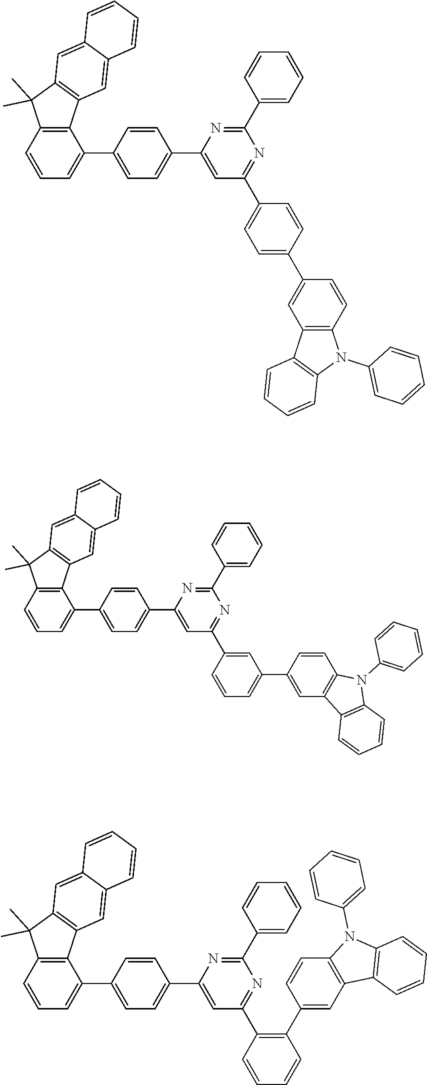 [SMILES: CC1(C)c2cc3ccccc3cc2-c2c(-c3ccc(-c4cc(-c5ccc(-c6ccc7c(c6)c6ccccc6n7-c6ccccc6)cc5)nc(-c5ccccc5)n4)cc3)cccc21 ; CC1(C)c2cc3ccccc3cc2-c2c(-c3ccc(-c4cc(-c5cccc(-c6ccc7c(c6)c6ccccc6n7-c6ccccc6)c5)nc(-c5ccccc5)n4)cc3)cccc21 ; CC1(C)c2cc3ccccc3cc2-c2c(-c3ccc(-c4cc(-c5ccccc5-c5ccc6c(c5)c5ccccc5n6-c5ccccc5)nc(-c5ccccc5)n4)cc3)cccc21]

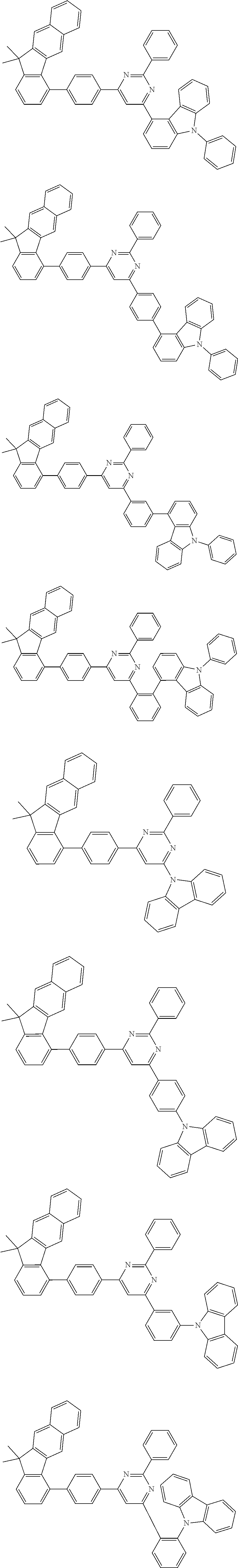

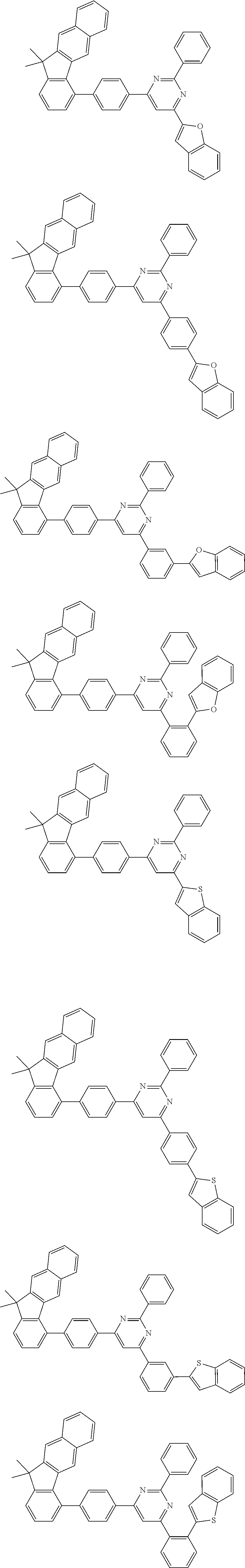

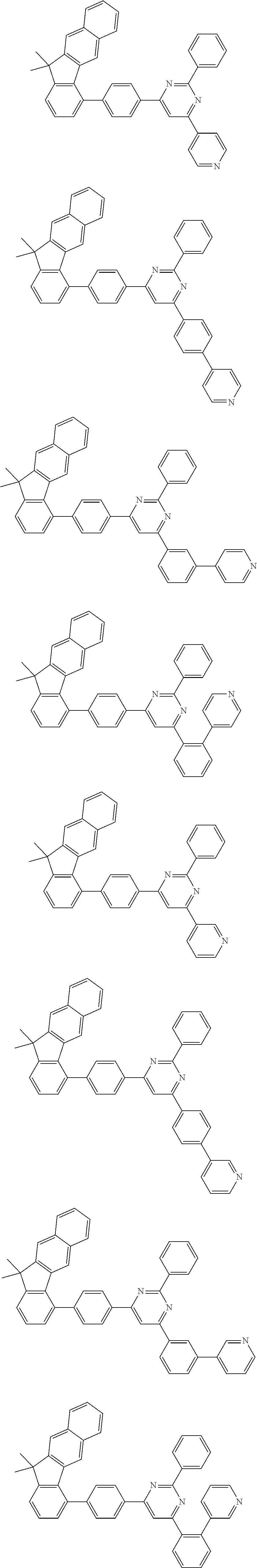

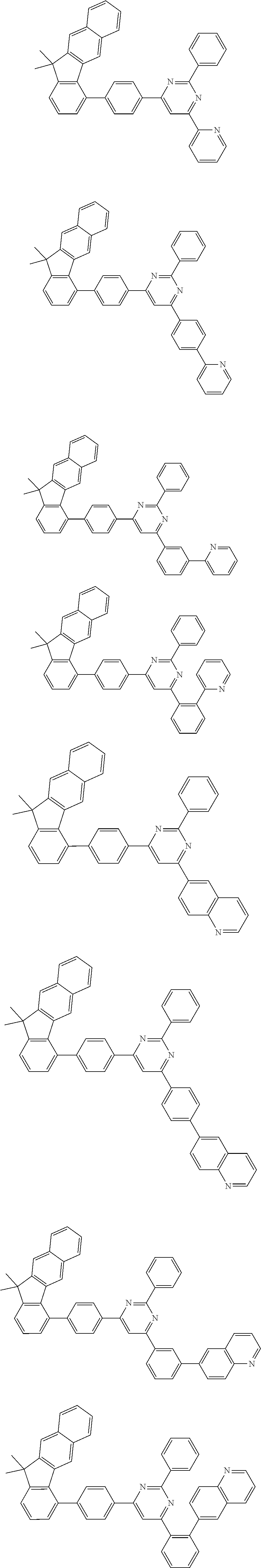

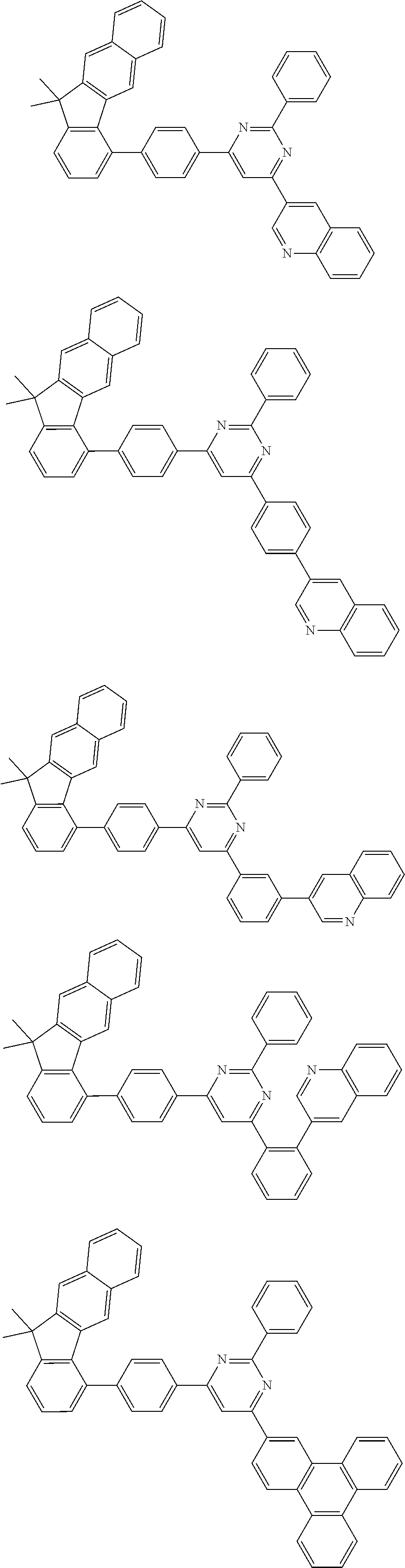 [SMILES: CC1(C)c2cc3ccccc3cc2-c2c(-c3ccc(-c4cc(-c5cnc6ccccc6c5)nc(-c5ccccc5)n4)cc3)cccc21 ; CC1(C)c2cc3ccccc3cc2-c2c(-c3ccc(-c4cc(-c5ccc(-c6cnc7ccccc7c6)cc5)nc(-c5ccccc5)n4)cc3)cccc21 ; CC1(C)c2cc3ccccc3cc2-c2c(-c3ccc(-c4cc(-c5cccc(-c6cnc7ccccc7c6)c5)nc(-c5ccccc5)n4)cc3)cccc21 ; CC1(C)c2cc3ccccc3cc2-c2c(-c3ccc(-c4cc(-c5ccccc5-c5cnc6ccccc6c5)nc(-c5ccccc5)n4)cc3)cccc21 ; CC1(C)c2cc3ccccc3cc2-c2c(-c3ccc(-c4cc(-c5ccc6c7ccccc7c7ccccc7c6c5)nc(-c5ccccc5)n4)cc3)cccc21]

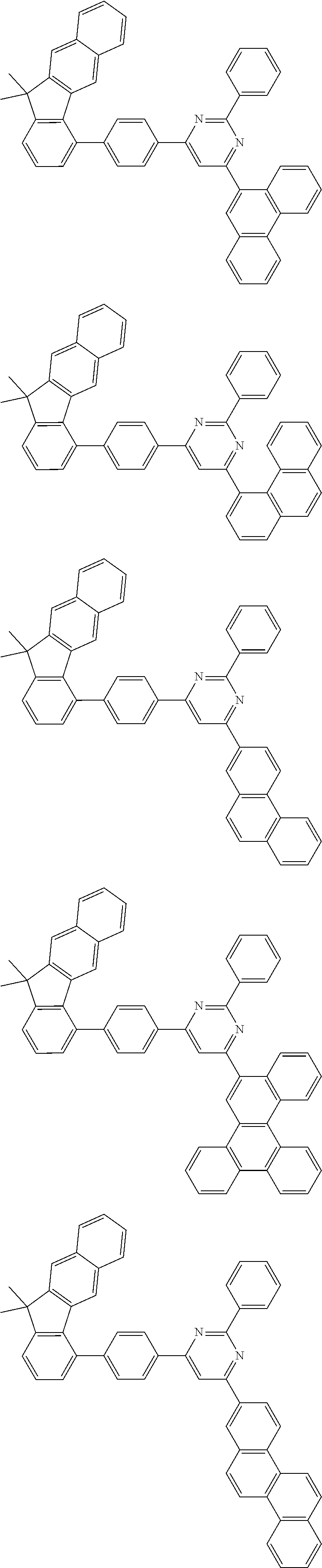 [SMILES: CC1(C)c2cc3ccccc3cc2-c2c(-c3ccc(-c4cc(-c5cc6ccccc6c6ccccc56)nc(-c5ccccc5)n4)cc3)cccc21 ; CC1(C)c2cc3ccccc3cc2-c2c(-c3ccc(-c4cc(-c5cccc6ccc7ccccc7c56)nc(-c5ccccc5)n4)cc3)cccc21 ; CC1(C)c2cc3ccccc3cc2-c2c(-c3ccc(-c4cc(-c5ccc6c(ccc7ccccc76)c5)nc(-c5ccccc5)n4)cc3)cccc21 ; CC1(C)c2cc3ccccc3cc2-c2c(-c3ccc(-c4cc(-c5cc6c7ccccc7c7ccccc7c6c6ccccc56)nc(-c5ccccc5)n4)cc3)cccc21 ; CC1(C)c2cc3ccccc3cc2-c2c(-c3ccc(-c4cc(-c5ccc6c(ccc7c8ccccc8ccc67)c5)nc(-c5ccccc5)n4)cc3)cccc21]

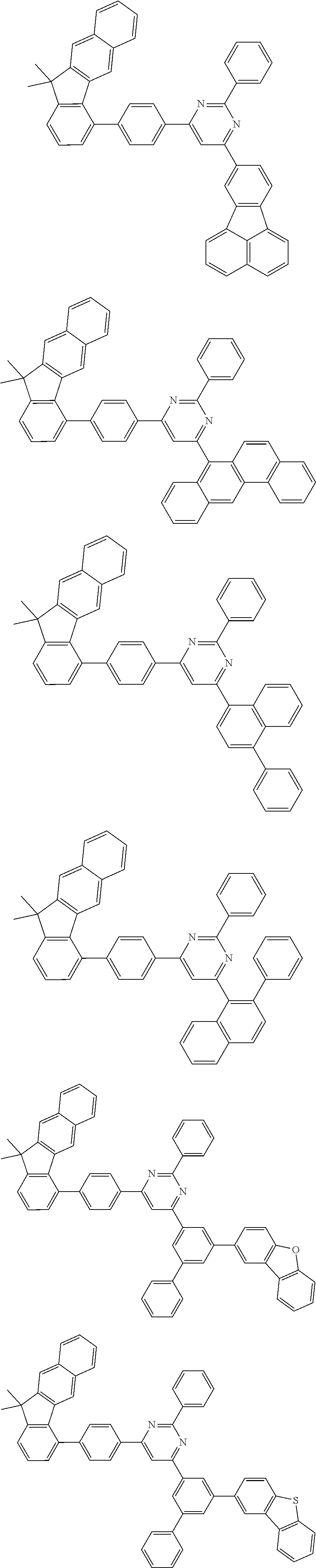

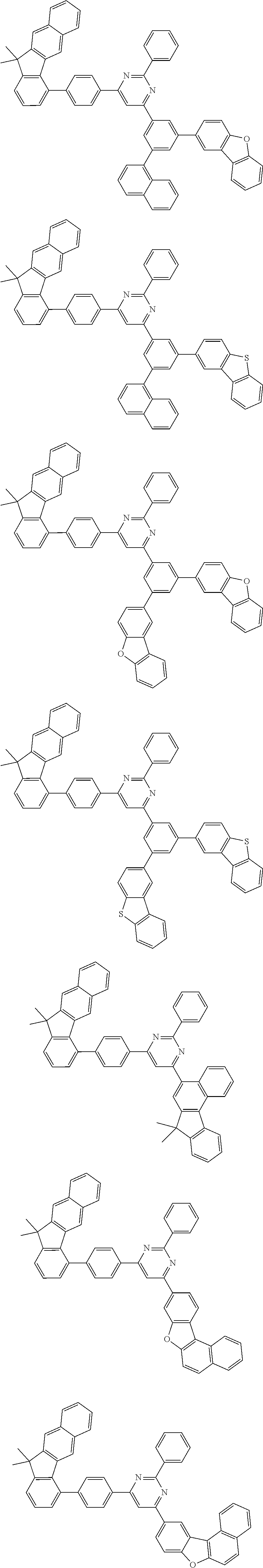

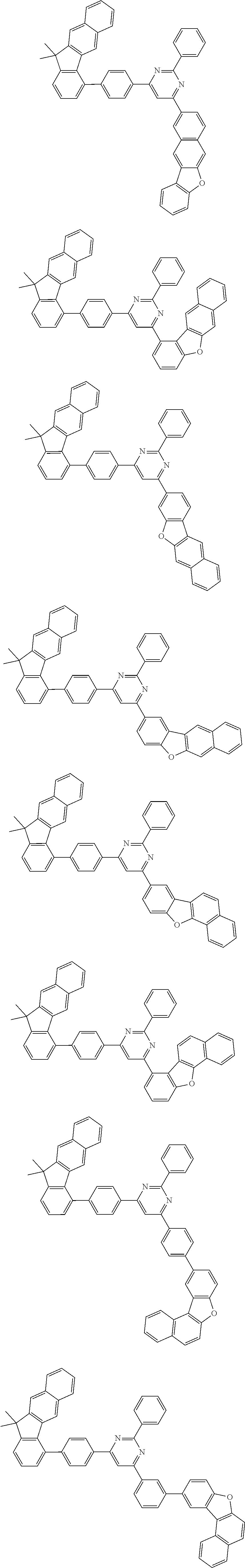

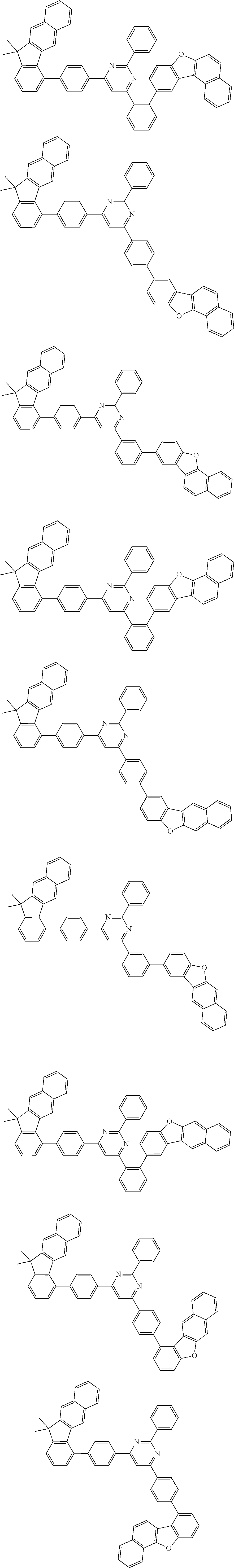

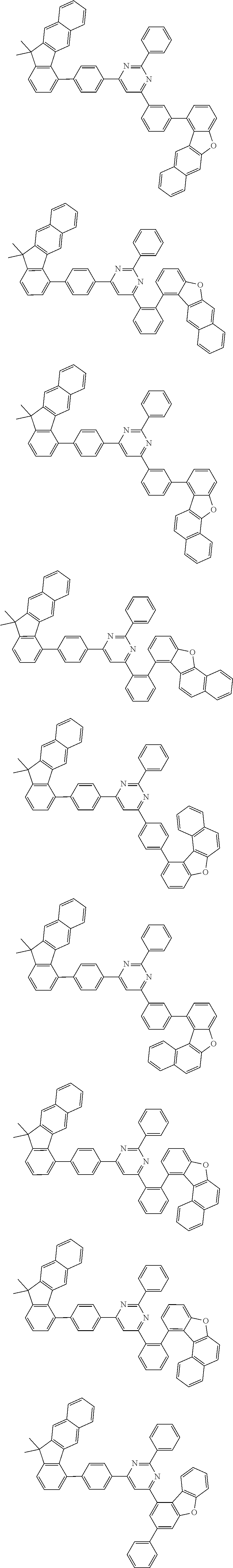

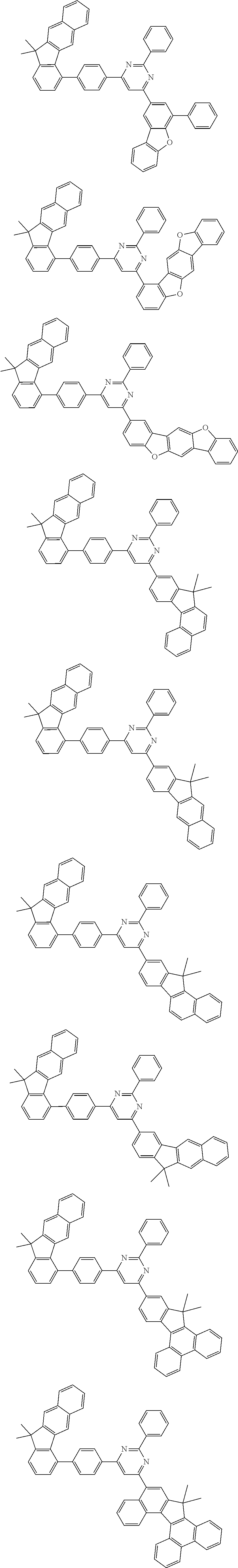

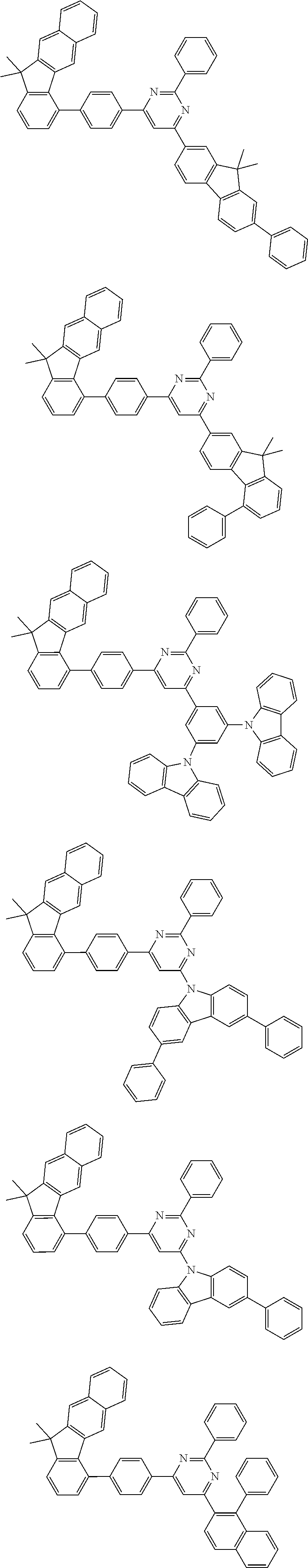

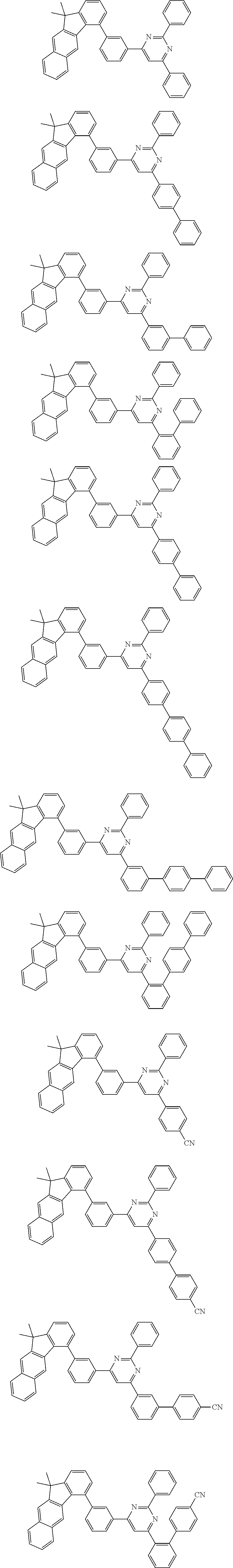

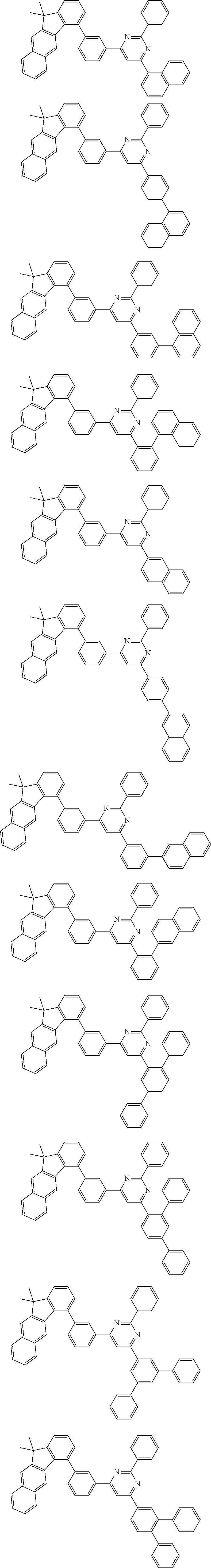

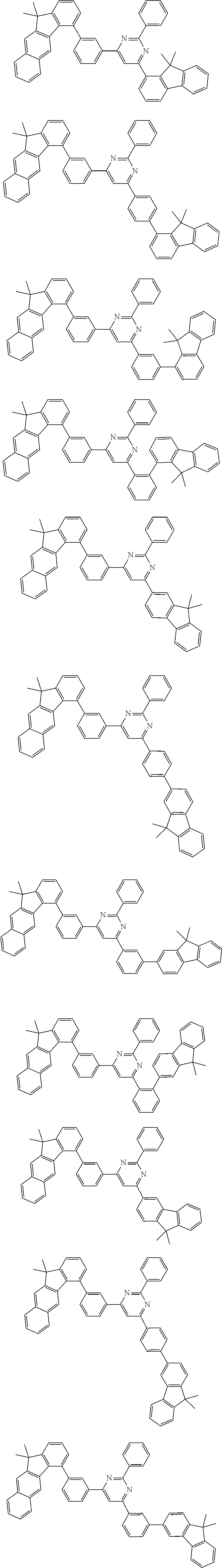

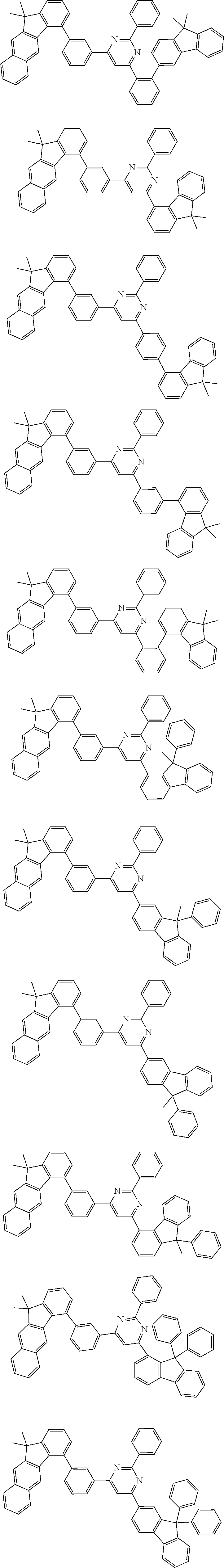

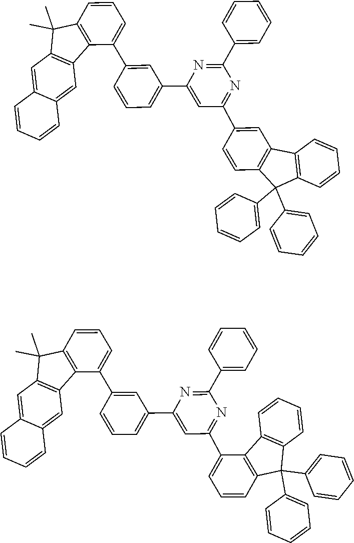 [SMILES: CC1(C)c2cc3ccccc3cc2-c2c(-c3cccc(-c4cc(-c5ccc6c(c5)-c5ccccc5C6(c5ccccc5)c5ccccc5)nc(-c5ccccc5)n4)c3)cccc21 ; CC1(C)c2cc3ccccc3cc2-c2c(-c3cccc(-c4cc(-c5cccc6c5-c5ccccc5C6(c5ccccc5)c5ccccc5)nc(-c5ccccc5)n4)c3)cccc21]

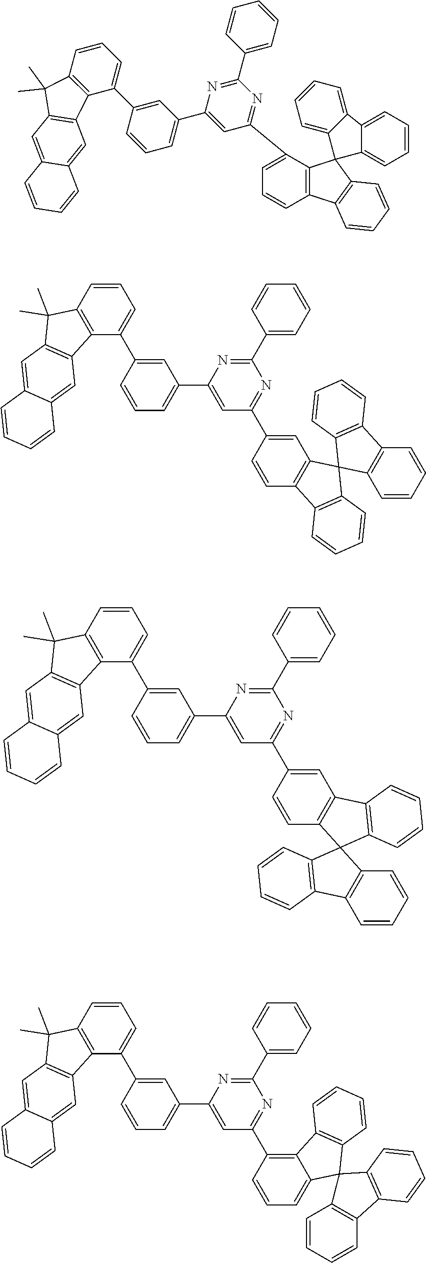 [SMILES: CC1(C)c2cc3ccccc3cc2-c2c(-c3cccc(-c4cc(-c5cccc6c5C5(c7ccccc7-c7ccccc75)c5ccccc5-6)nc(-c5ccccc5)n4)c3)cccc21 ; CC1(C)c2cc3ccccc3cc2-c2c(-c3cccc(-c4cc(-c5ccc6c(c5)C5(c7ccccc7-c7ccccc75)c5ccccc5-6)nc(-c5ccccc5)n4)c3)cccc21 ; CC1(C)c2cc3ccccc3cc2-c2c(-c3cccc(-c4cc(-c5ccc6c(c5)-c5ccccc5C65c6ccccc6-c6ccccc65)nc(-c5ccccc5)n4)c3)cccc21 ; CC1(C)c2cc3ccccc3cc2-c2c(-c3cccc(-c4cc(-c5cccc6c5-c5ccccc5C65c6ccccc6-c6ccccc65)nc(-c5ccccc5)n4)c3)cccc21]

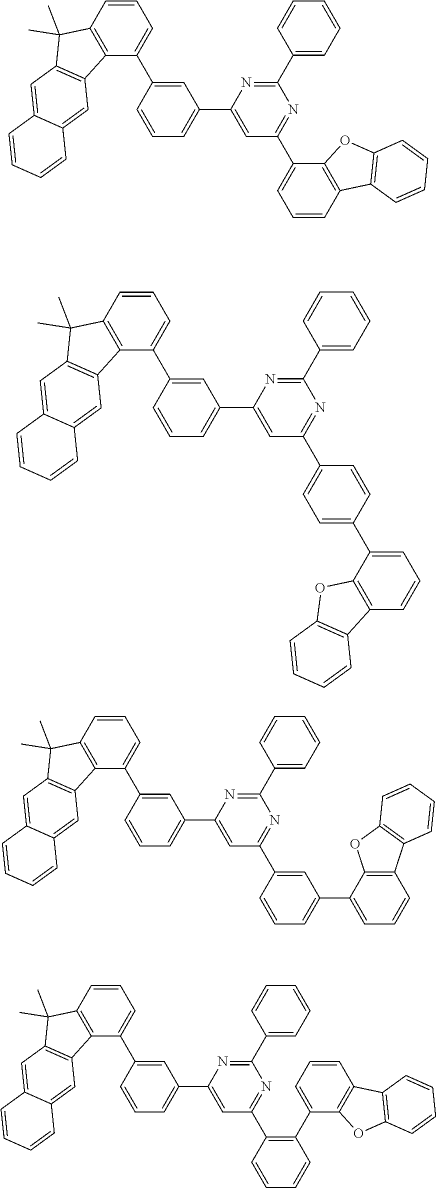

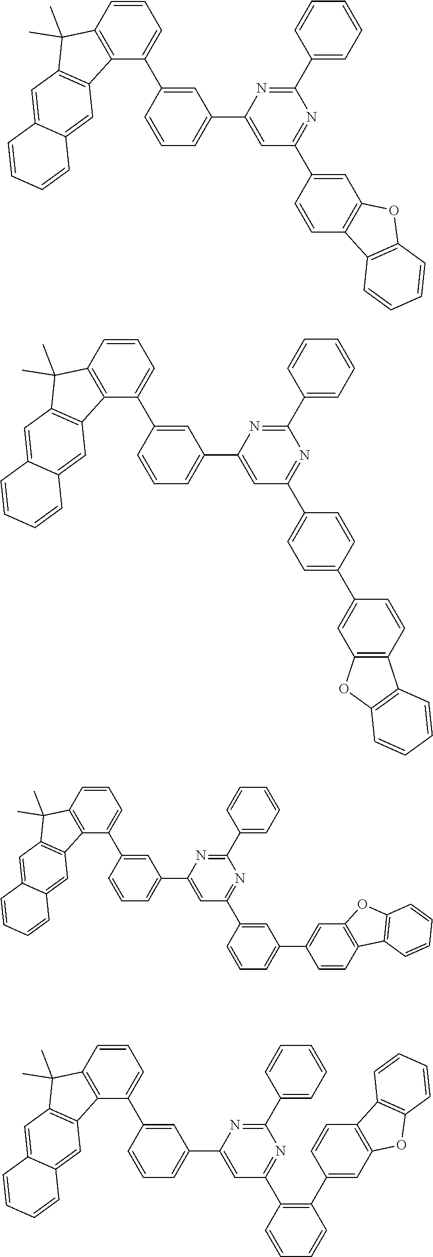 [SMILES: CC1(C)c2cc3ccccc3cc2-c2c(-c3cccc(-c4cc(-c5ccc6c(c5)oc5ccccc56)nc(-c5ccccc5)n4)c3)cccc21 ; CC1(C)c2cc3ccccc3cc2-c2c(-c3cccc(-c4cc(-c5ccc(-c6ccc7c(c6)oc6ccccc67)cc5)nc(-c5ccccc5)n4)c3)cccc21 ; CC1(C)c2cc3ccccc3cc2-c2c(-c3cccc(-c4cc(-c5cccc(-c6ccc7c(c6)oc6ccccc67)c5)nc(-c5ccccc5)n4)c3)cccc21 ; CC1(C)c2cc3ccccc3cc2-c2c(-c3cccc(-c4cc(-c5ccccc5-c5ccc6c(c5)oc5ccccc56)nc(-c5ccccc5)n4)c3)cccc21]

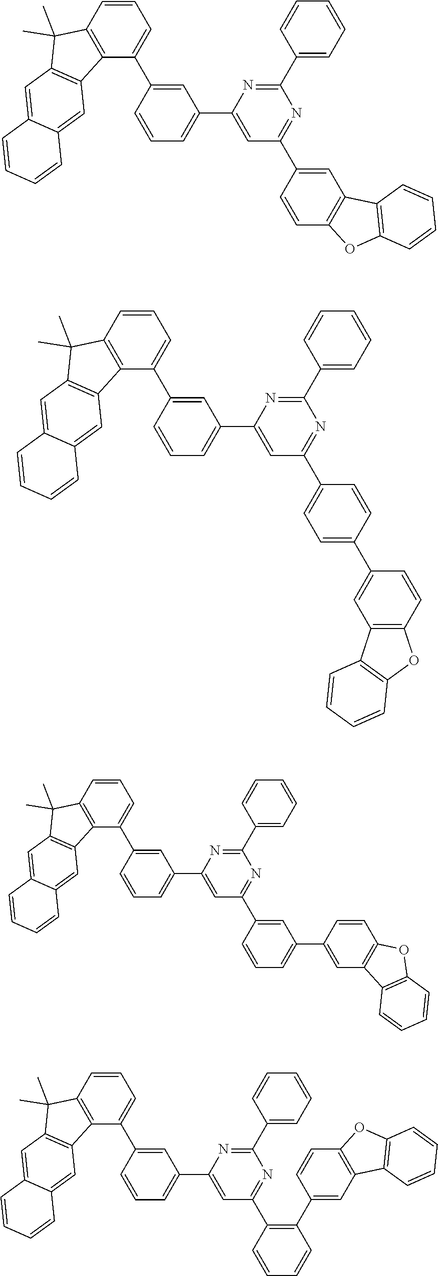 [SMILES: CC1(C)c2cc3ccccc3cc2-c2c(-c3cccc(-c4cc(-c5ccc6oc7ccccc7c6c5)nc(-c5ccccc5)n4)c3)cccc21 ; CC1(C)c2cc3ccccc3cc2-c2c(-c3cccc(-c4cc(-c5ccc(-c6ccc7oc8ccccc8c7c6)cc5)nc(-c5ccccc5)n4)c3)cccc21 ; CC1(C)c2cc3ccccc3cc2-c2c(-c3cccc(-c4cc(-c5cccc(-c6ccc7oc8ccccc8c7c6)c5)nc(-c5ccccc5)n4)c3)cccc21 ; CC1(C)c2cc3ccccc3cc2-c2c(-c3cccc(-c4cc(-c5ccccc5-c5ccc6oc7ccccc7c6c5)nc(-c5ccccc5)n4)c3)cccc21]

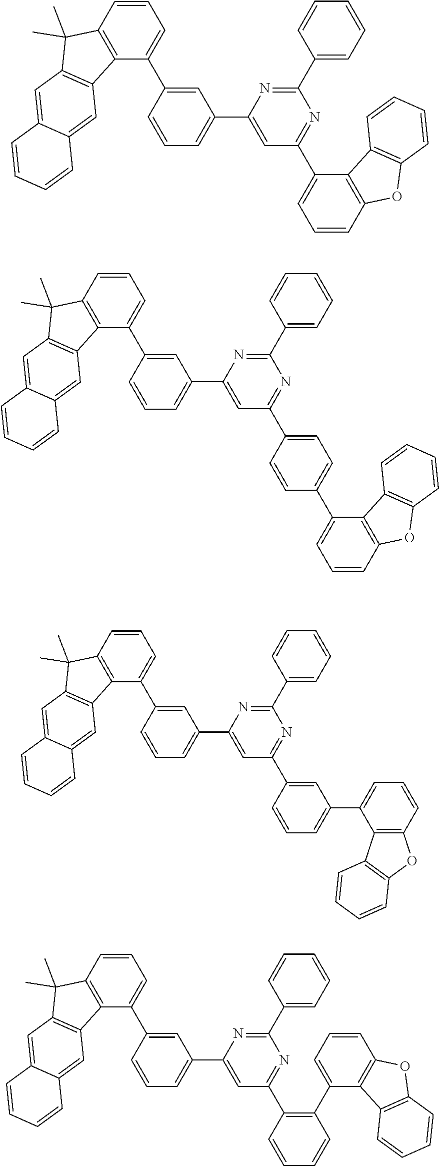

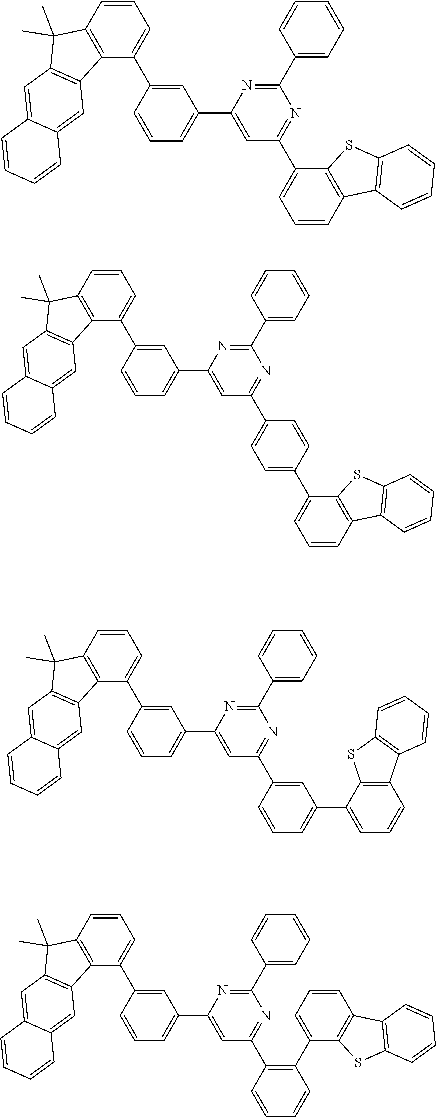 [SMILES: CC1(C)c2cc3ccccc3cc2-c2c(-c3cccc(-c4cc(-c5cccc6c5sc5ccccc56)nc(-c5ccccc5)n4)c3)cccc21 ; CC1(C)c2cc3ccccc3cc2-c2c(-c3cccc(-c4cc(-c5ccc(-c6cccc7c6sc6ccccc67)cc5)nc(-c5ccccc5)n4)c3)cccc21 ; CC1(C)c2cc3ccccc3cc2-c2c(-c3cccc(-c4cc(-c5cccc(-c6cccc7c6sc6ccccc67)c5)nc(-c5ccccc5)n4)c3)cccc21 ; CC1(C)c2cc3ccccc3cc2-c2c(-c3cccc(-c4cc(-c5ccccc5-c5cccc6c5sc5ccccc56)nc(-c5ccccc5)n4)c3)cccc21]

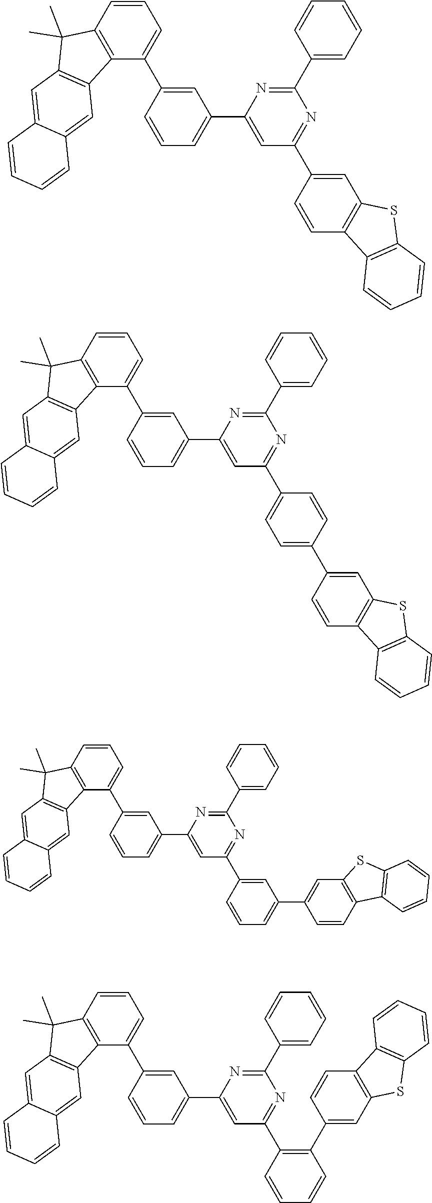 [SMILES: CC1(C)c2cc3ccccc3cc2-c2c(-c3cccc(-c4cc(-c5ccc6c(c5)sc5ccccc56)nc(-c5ccccc5)n4)c3)cccc21 ; CC1(C)c2cc3ccccc3cc2-c2c(-c3cccc(-c4cc(-c5ccc(-c6ccc7c(c6)sc6ccccc67)cc5)nc(-c5ccccc5)n4)c3)cccc21 ; CC1(C)c2cc3ccccc3cc2-c2c(-c3cccc(-c4cc(-c5cccc(-c6ccc7c(c6)sc6ccccc67)c5)nc(-c5ccccc5)n4)c3)cccc21 ; CC1(C)c2cc3ccccc3cc2-c2c(-c3cccc(-c4cc(-c5ccccc5-c5ccc6c(c5)sc5ccccc56)nc(-c5ccccc5)n4)c3)cccc21]

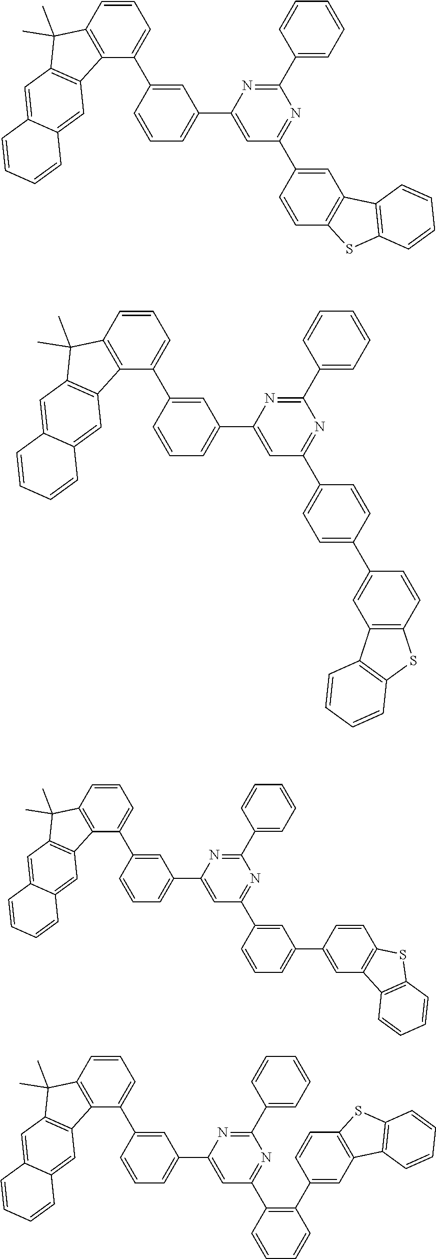 [SMILES: CC1(C)c2cc3ccccc3cc2-c2c(-c3cccc(-c4cc(-c5ccc6sc7ccccc7c6c5)nc(-c5ccccc5)n4)c3)cccc21 ; CC1(C)c2cc3ccccc3cc2-c2c(-c3cccc(-c4cc(-c5ccc(-c6ccc7sc8ccccc8c7c6)cc5)nc(-c5ccccc5)n4)c3)cccc21 ; CC1(C)c2cc3ccccc3cc2-c2c(-c3cccc(-c4cc(-c5cccc(-c6ccc7sc8ccccc8c7c6)c5)nc(-c5ccccc5)n4)c3)cccc21 ; CC1(C)c2cc3ccccc3cc2-c2c(-c3cccc(-c4cc(-c5ccccc5-c5ccc6sc7ccccc7c6c5)nc(-c5ccccc5)n4)c3)cccc21]

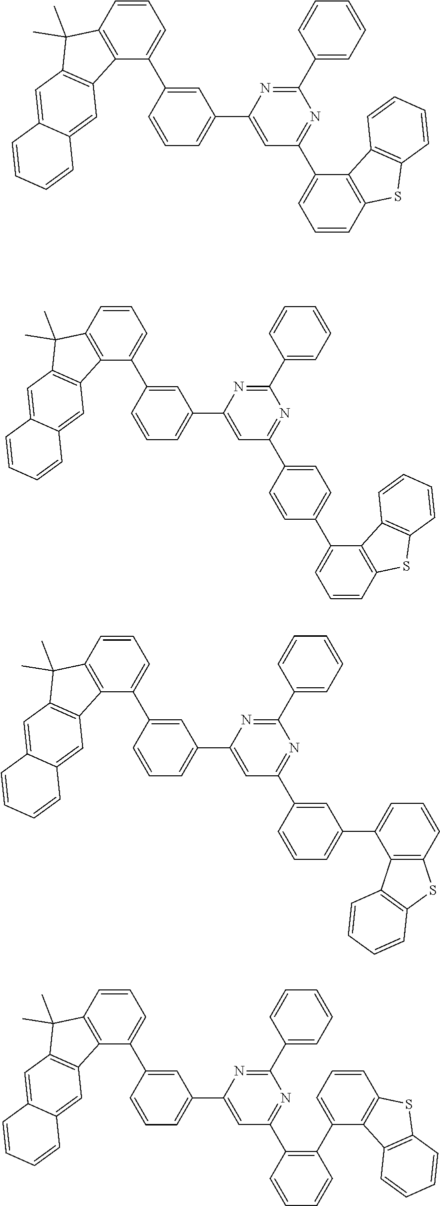

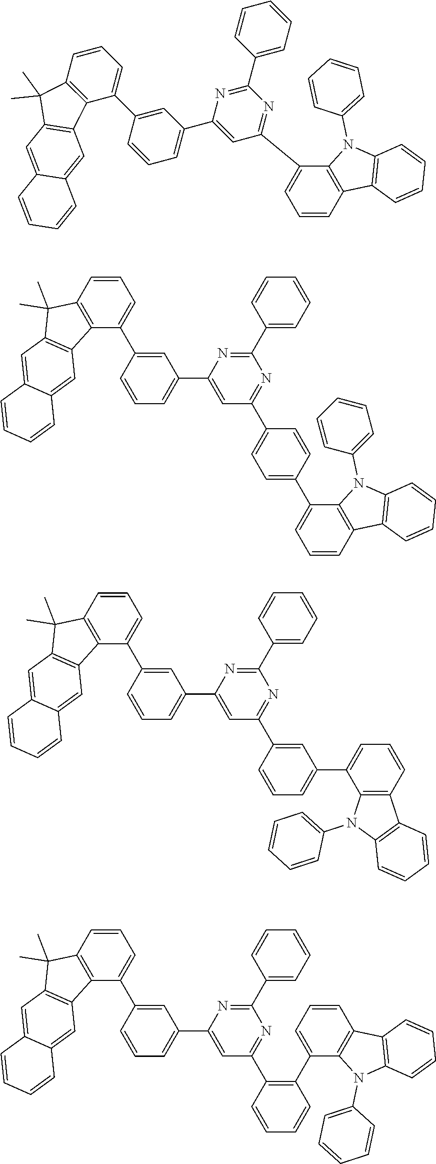

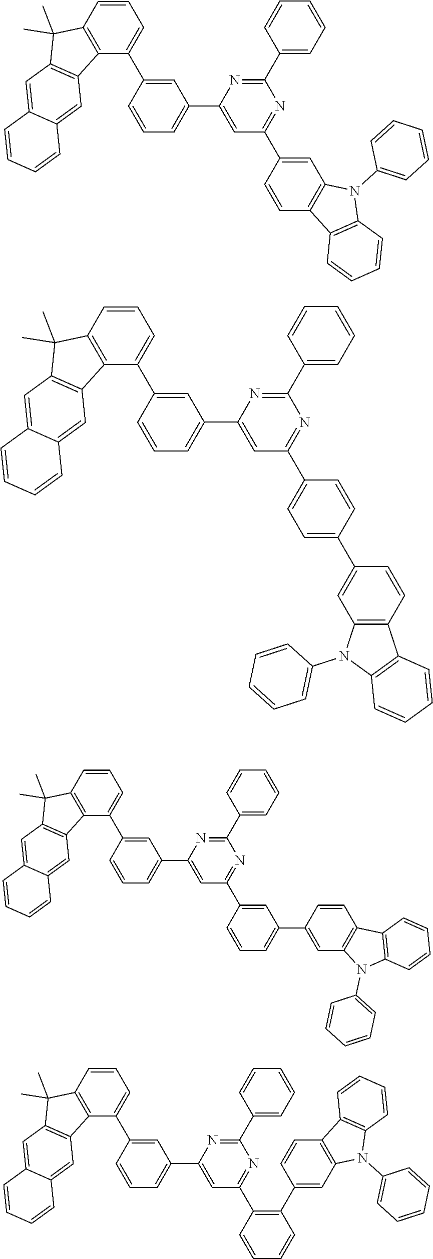

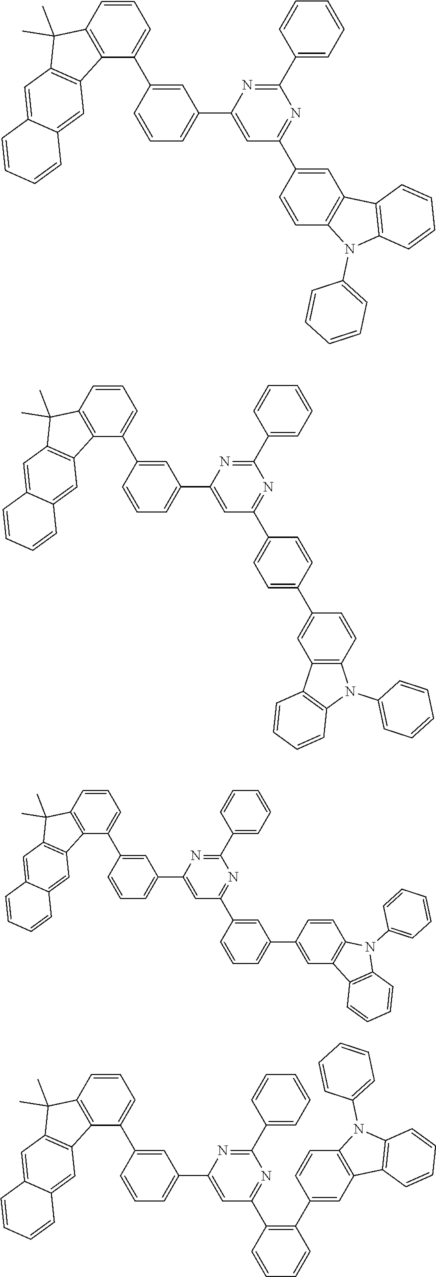

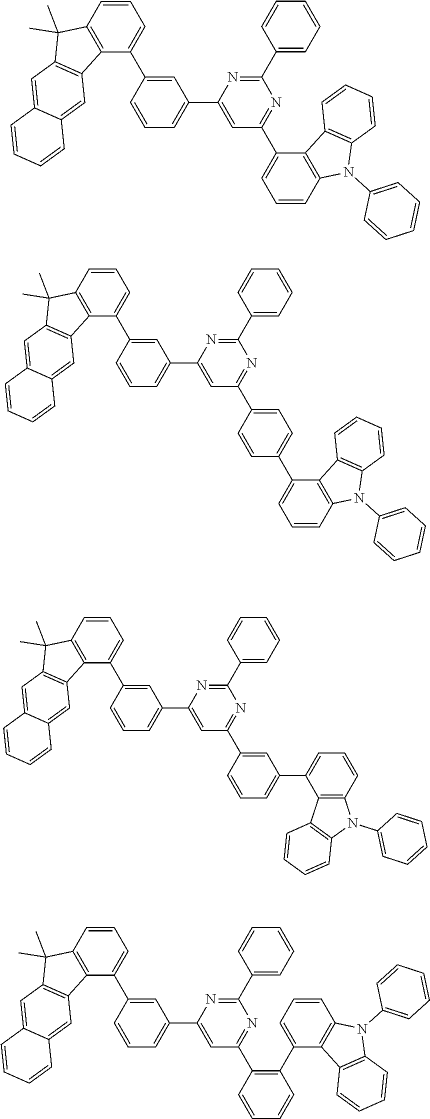 [SMILES: CC1(C)c2cc3ccccc3cc2-c2c(-c3cccc(-c4cc(-c5cccc6c5c5ccccc5n6-c5ccccc5)nc(-c5ccccc5)n4)c3)cccc21 ; CC1(C)c2cc3ccccc3cc2-c2c(-c3cccc(-c4cc(-c5ccc(-c6cccc7c6c6ccccc6n7-c6ccccc6)cc5)nc(-c5ccccc5)n4)c3)cccc21 ; CC1(C)c2cc3ccccc3cc2-c2c(-c3cccc(-c4cc(-c5cccc(-c6cccc7c6c6ccccc6n7-c6ccccc6)c5)nc(-c5ccccc5)n4)c3)cccc21 ; CC1(C)c2cc3ccccc3cc2-c2c(-c3cccc(-c4cc(-c5ccccc5-c5cccc6c5c5ccccc5n6-c5ccccc5)nc(-c5ccccc5)n4)c3)cccc21]

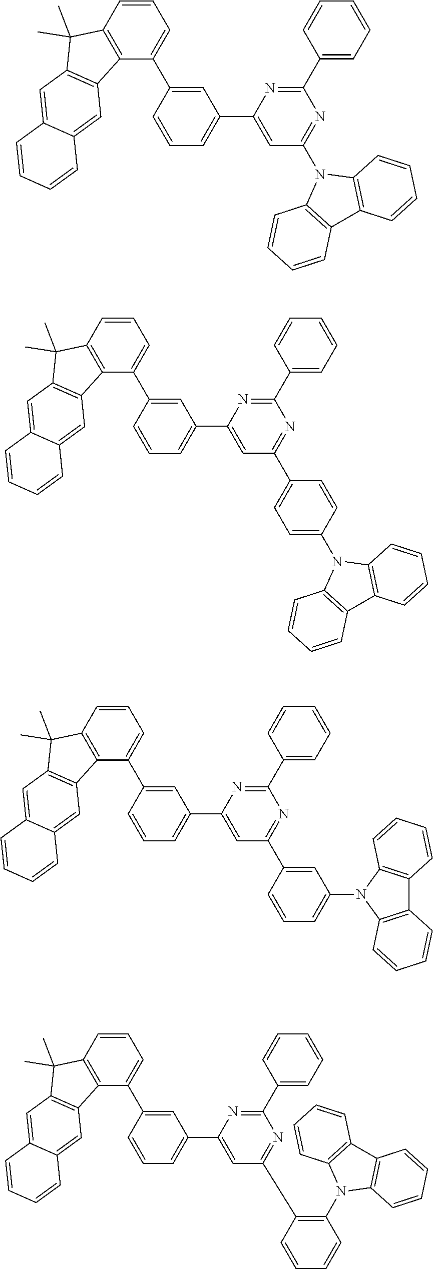 [SMILES: CC1(C)c2cc3ccccc3cc2-c2c(-c3cccc(-c4cc(-n5c6ccccc6c6ccccc65)nc(-c5ccccc5)n4)c3)cccc21 ; CC1(C)c2cc3ccccc3cc2-c2c(-c3cccc(-c4cc(-c5ccc(-n6c7ccccc7c7ccccc76)cc5)nc(-c5ccccc5)n4)c3)cccc21 ; CC1(C)c2cc3ccccc3cc2-c2c(-c3cccc(-c4cc(-c5cccc(-n6c7ccccc7c7ccccc76)c5)nc(-c5ccccc5)n4)c3)cccc21 ; CC1(C)c2cc3ccccc3cc2-c2c(-c3cccc(-c4cc(-c5ccccc5-n5c6ccccc6c6ccccc65)nc(-c5ccccc5)n4)c3)cccc21]

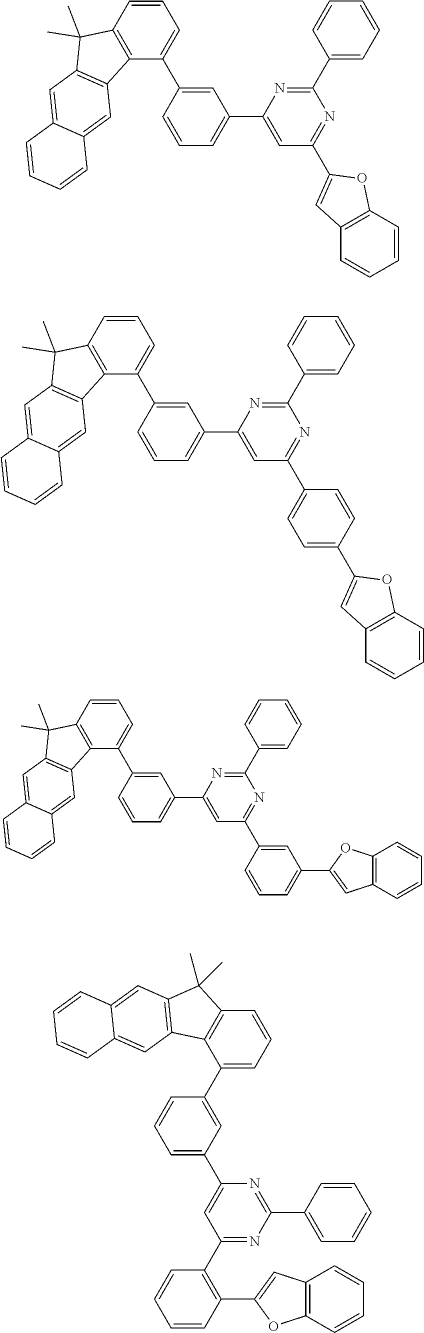 [SMILES: CC1(C)c2cc3ccccc3cc2-c2c(-c3cccc(-c4cc(-c5cc6ccccc6o5)nc(-c5ccccc5)n4)c3)cccc21 ; CC1(C)c2cc3ccccc3cc2-c2c(-c3cccc(-c4cc(-c5ccc(-c6cc7ccccc7o6)cc5)nc(-c5ccccc5)n4)c3)cccc21 ; CC1(C)c2cc3ccccc3cc2-c2c(-c3cccc(-c4cc(-c5cccc(-c6cc7ccccc7o6)c5)nc(-c5ccccc5)n4)c3)cccc21 ; CC1(C)c2cc3ccccc3cc2-c2c(-c3cccc(-c4cc(-c5ccccc5-c5cc6ccccc6o5)nc(-c5ccccc5)n4)c3)cccc21]

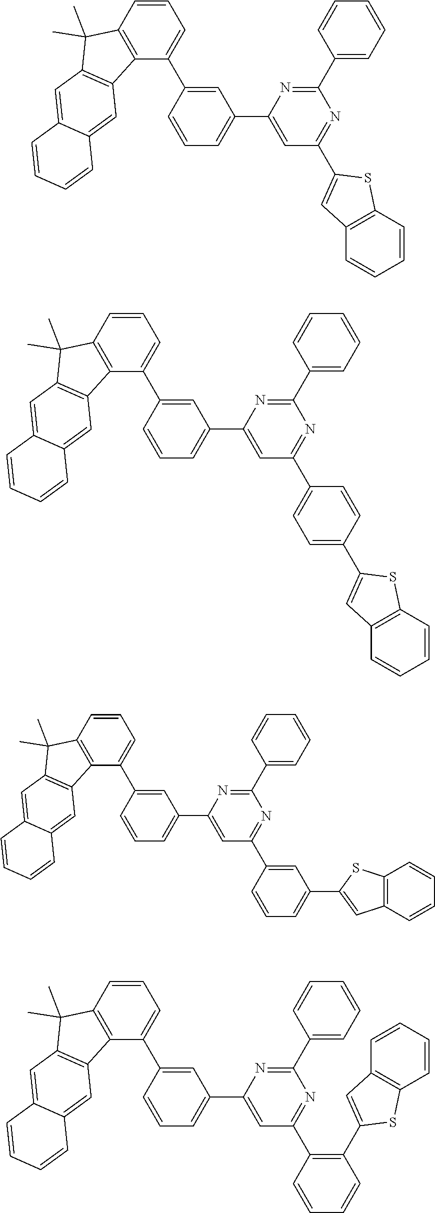 [SMILES: CC1(C)c2cc3ccccc3cc2-c2c(-c3cccc(-c4cc(-c5cc6ccccc6s5)nc(-c5ccccc5)n4)c3)cccc21 ; CC1(C)c2cc3ccccc3cc2-c2c(-c3cccc(-c4cc(-c5ccc(-c6cc7ccccc7s6)cc5)nc(-c5ccccc5)n4)c3)cccc21 ; CC1(C)c2cc3ccccc3cc2-c2c(-c3cccc(-c4cc(-c5cccc(-c6cc7ccccc7s6)c5)nc(-c5ccccc5)n4)c3)cccc21 ; CC1(C)c2cc3ccccc3cc2-c2c(-c3cccc(-c4cc(-c5ccccc5-c5cc6ccccc6s5)nc(-c5ccccc5)n4)c3)cccc21]

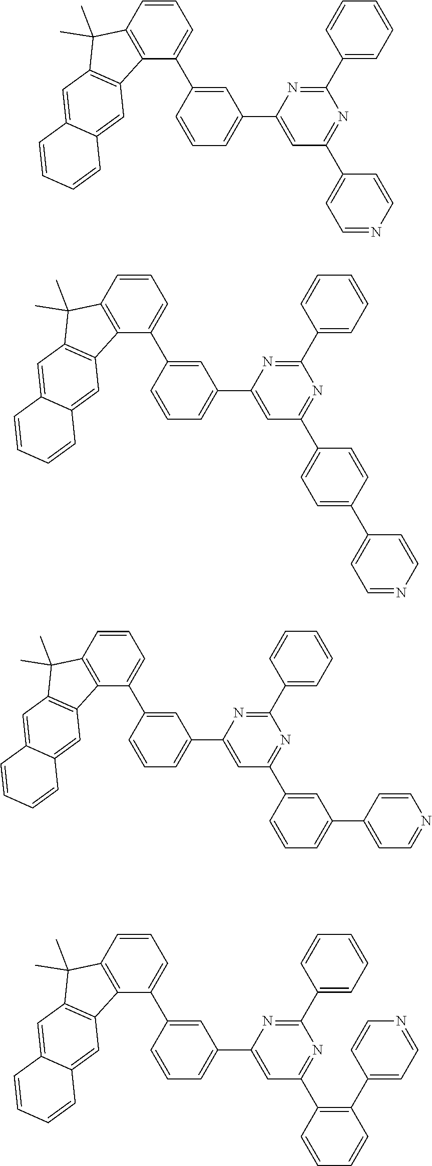

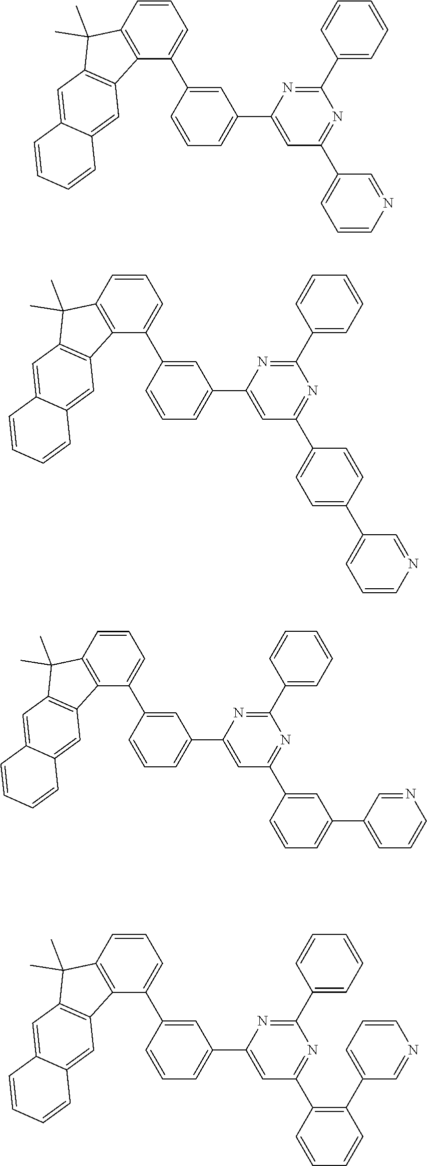

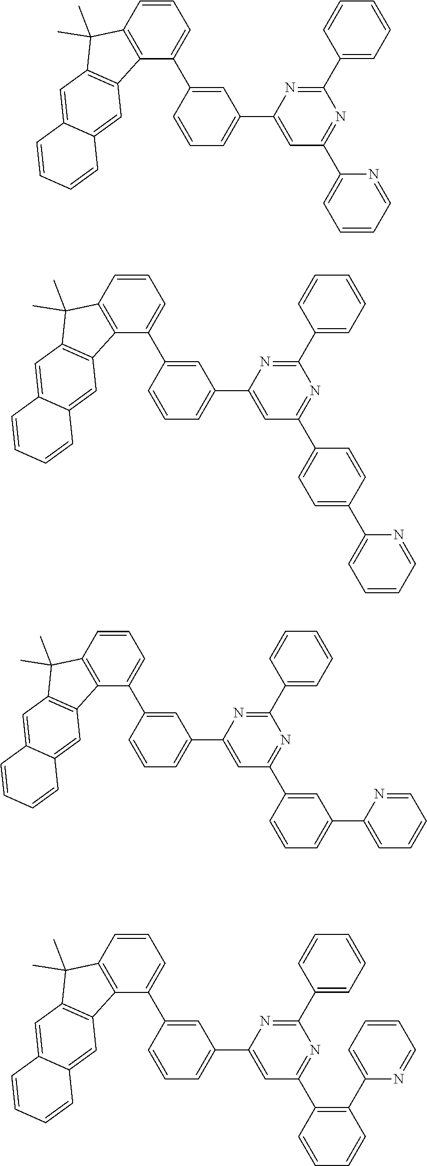

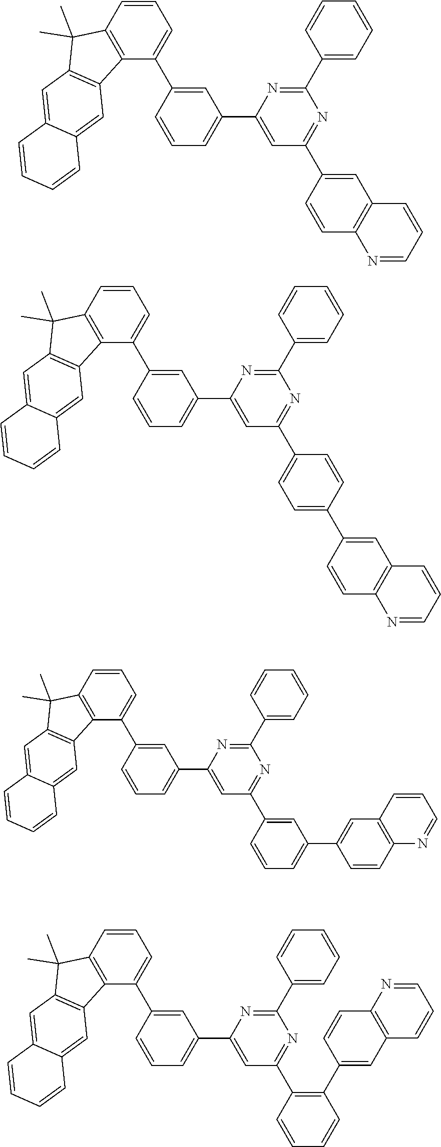 [SMILES: CC1(C)c2cc3ccccc3cc2-c2c(-c3cccc(-c4cc(-c5ccc6ncccc6c5)nc(-c5ccccc5)n4)c3)cccc21 ; CC1(C)c2cc3ccccc3cc2-c2c(-c3cccc(-c4cc(-c5ccc(-c6ccc7ncccc7c6)cc5)nc(-c5ccccc5)n4)c3)cccc21 ; CC1(C)c2cc3ccccc3cc2-c2c(-c3cccc(-c4cc(-c5cccc(-c6ccc7ncccc7c6)c5)nc(-c5ccccc5)n4)c3)cccc21 ; CC1(C)c2cc3ccccc3cc2-c2c(-c3cccc(-c4cc(-c5ccccc5-c5ccc6ncccc6c5)nc(-c5ccccc5)n4)c3)cccc21]

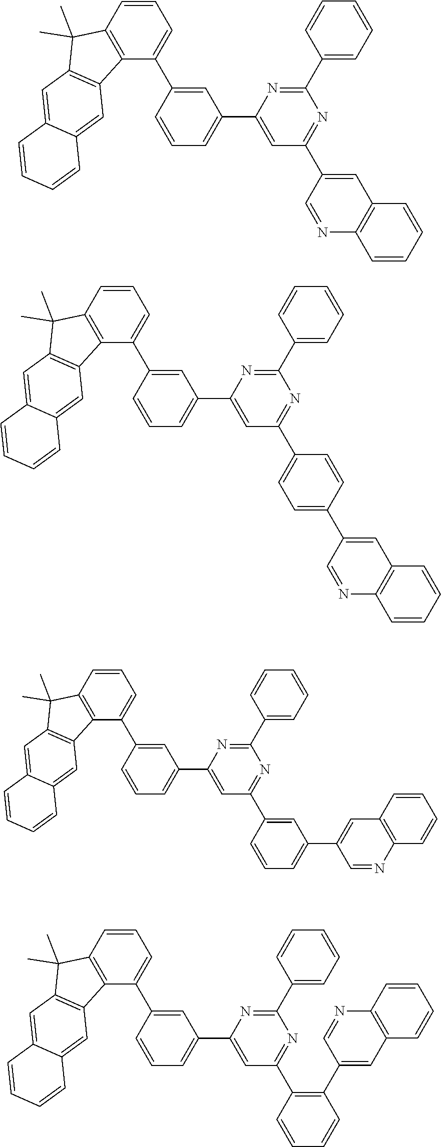 [SMILES: CC1(C)c2cc3ccccc3cc2-c2c(-c3cccc(-c4cc(-c5cnc6ccccc6c5)nc(-c5ccccc5)n4)c3)cccc21 ; CC1(C)c2cc3ccccc3cc2-c2c(-c3cccc(-c4cc(-c5ccc(-c6cnc7ccccc7c6)cc5)nc(-c5ccccc5)n4)c3)cccc21 ; CC1(C)c2cc3ccccc3cc2-c2c(-c3cccc(-c4cc(-c5cccc(-c6cnc7ccccc7c6)c5)nc(-c5ccccc5)n4)c3)cccc21 ; CC1(C)c2cc3ccccc3cc2-c2c(-c3cccc(-c4cc(-c5ccccc5-c5cnc6ccccc6c5)nc(-c5ccccc5)n4)c3)cccc21]

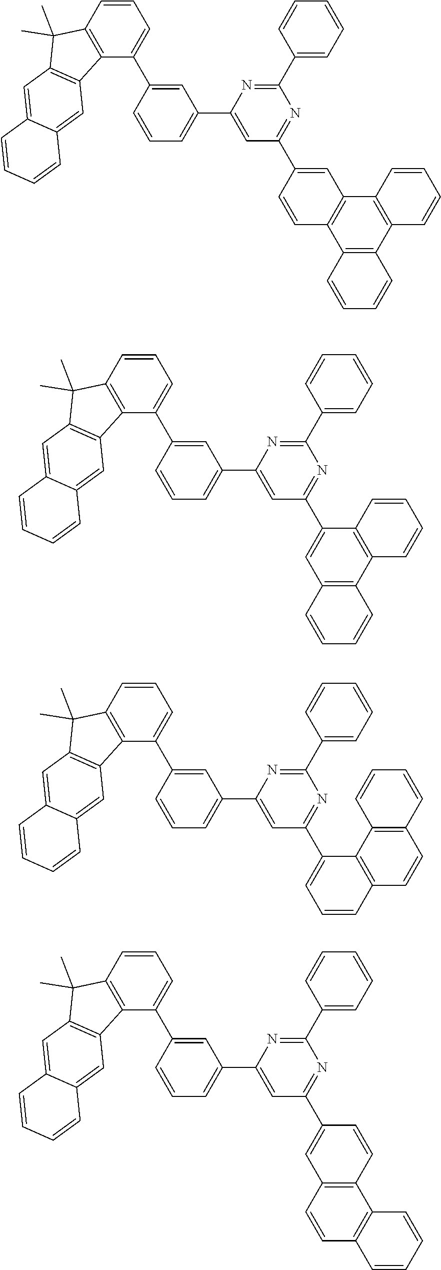

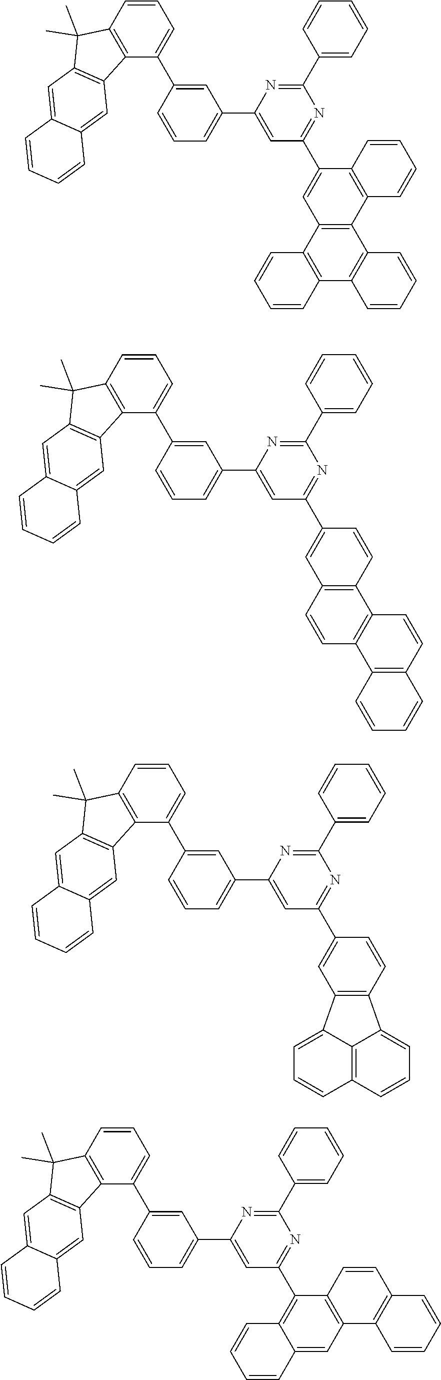

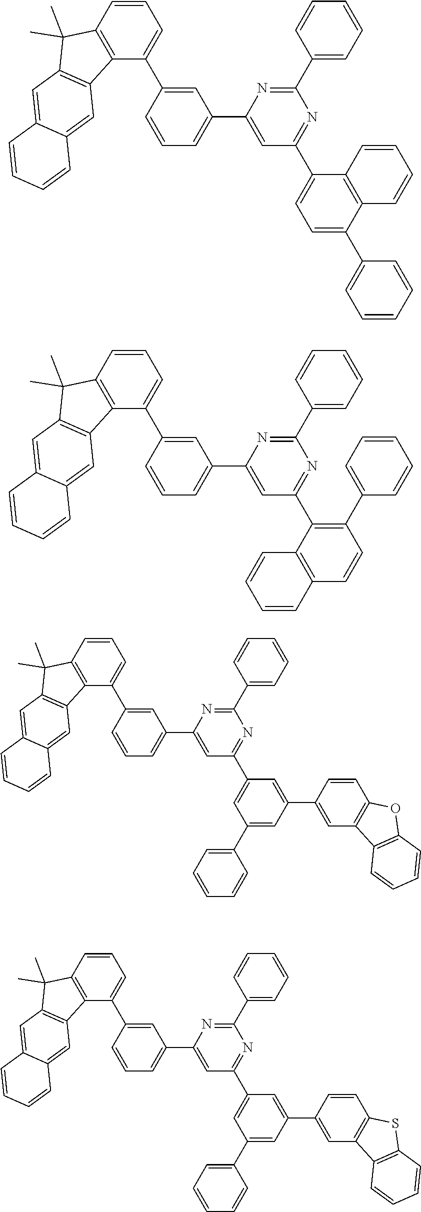

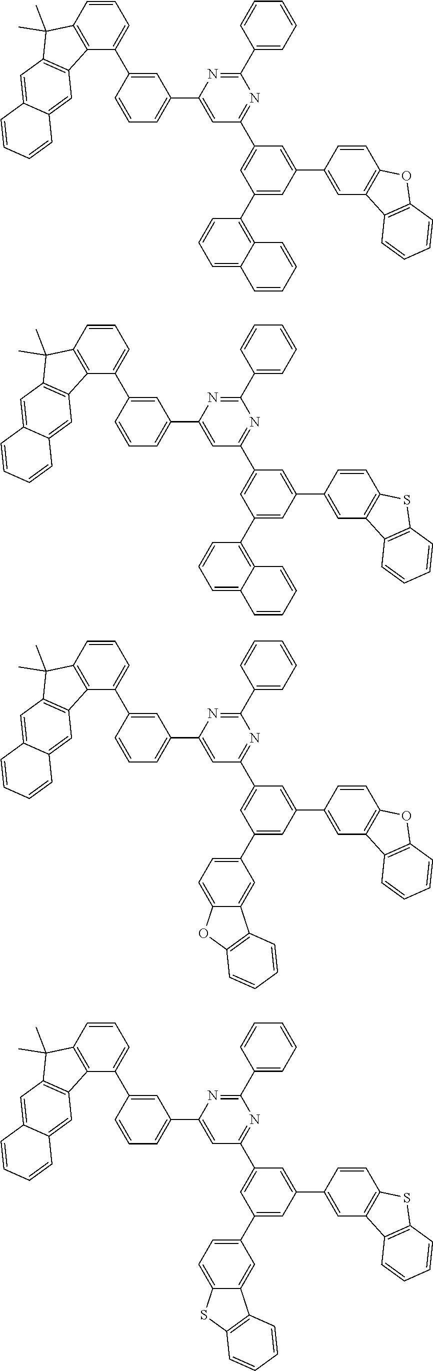

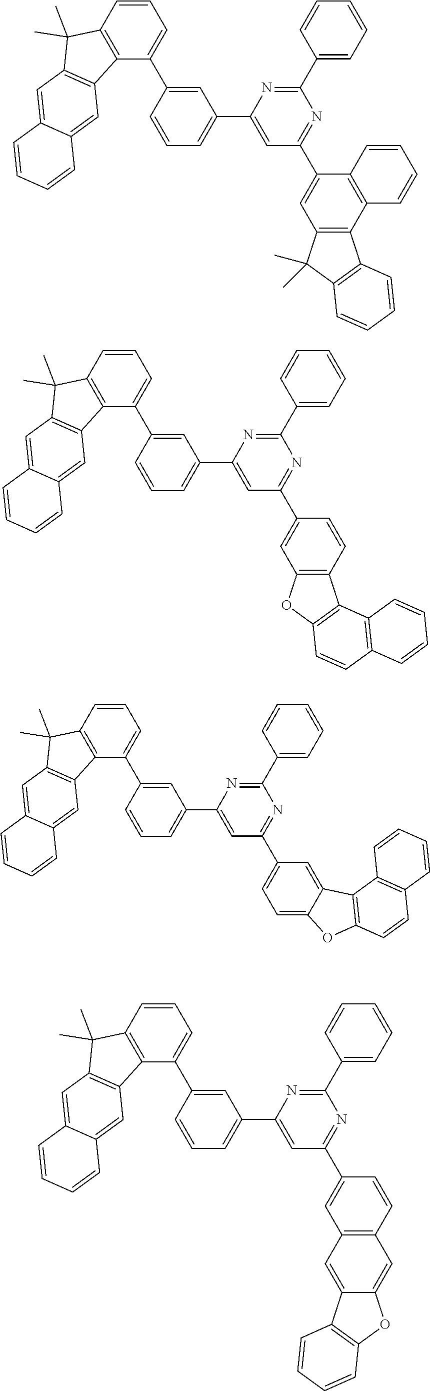

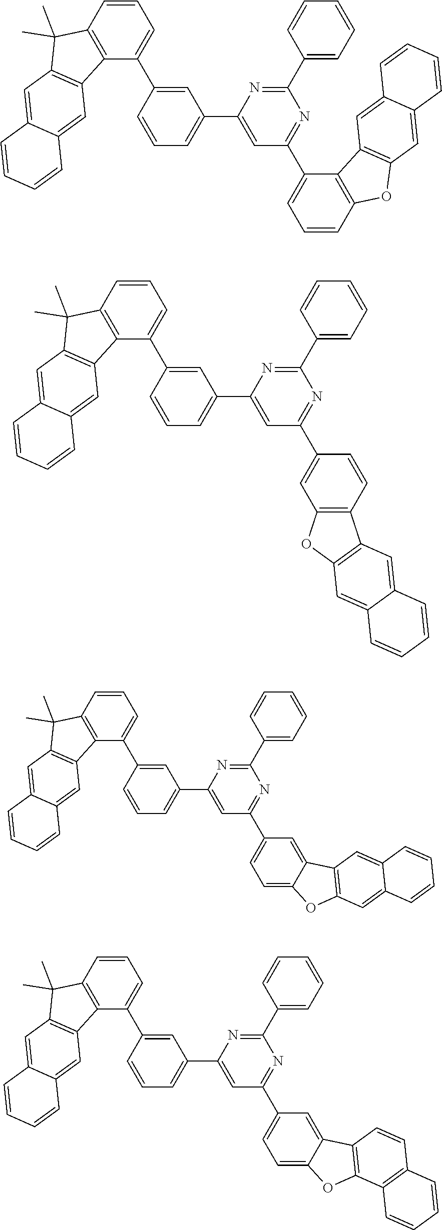 [SMILES: CC1(C)c2cc3ccccc3cc2-c2c(-c3cccc(-c4cc(-c5cccc6oc7cc8ccccc8cc7c56)nc(-c5ccccc5)n4)c3)cccc21 ; CC1(C)c2cc3ccccc3cc2-c2c(-c3cccc(-c4cc(-c5ccc6c(c5)oc5cc7ccccc7cc56)nc(-c5ccccc5)n4)c3)cccc21 ; CC1(C)c2cc3ccccc3cc2-c2c(-c3cccc(-c4cc(-c5ccc6oc7cc8ccccc8cc7c6c5)nc(-c5ccccc5)n4)c3)cccc21 ; CC1(C)c2cc3ccccc3cc2-c2c(-c3cccc(-c4cc(-c5ccc6oc7c8ccccc8ccc7c6c5)nc(-c5ccccc5)n4)c3)cccc21]

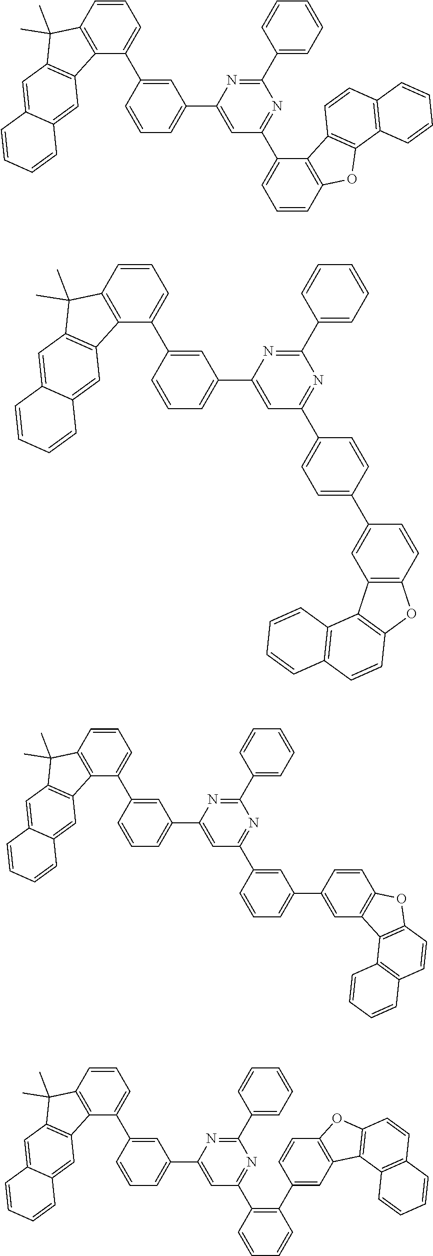 [SMILES: CC1(C)c2cc3ccccc3cc2-c2c(-c3cccc(-c4cc(-c5cccc6oc7c8ccccc8ccc7c56)nc(-c5ccccc5)n4)c3)cccc21 ; CC1(C)c2cc3ccccc3cc2-c2c(-c3cccc(-c4cc(-c5ccc(-c6ccc7oc8ccc9ccccc9c8c7c6)cc5)nc(-c5ccccc5)n4)c3)cccc21 ; CC1(C)c2cc3ccccc3cc2-c2c(-c3cccc(-c4cc(-c5cccc(-c6ccc7oc8ccc9ccccc9c8c7c6)c5)nc(-c5ccccc5)n4)c3)cccc21 ; CC1(C)c2cc3ccccc3cc2-c2c(-c3cccc(-c4cc(-c5ccccc5-c5ccc6oc7ccc8ccccc8c7c6c5)nc(-c5ccccc5)n4)c3)cccc21]

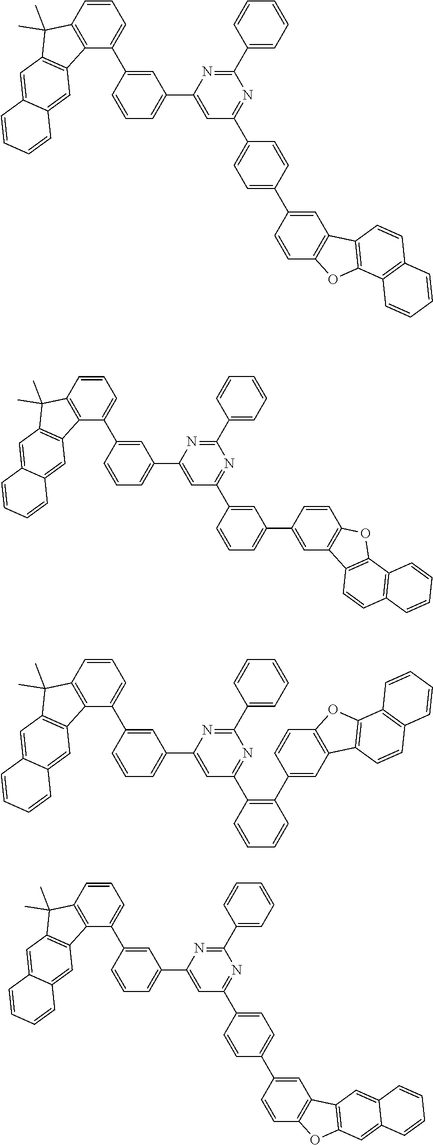 [SMILES: CC1(C)c2cc3ccccc3cc2-c2c(-c3cccc(-c4cc(-c5ccc(-c6ccc7oc8c9ccccc9ccc8c7c6)cc5)nc(-c5ccccc5)n4)c3)cccc21 ; CC1(C)c2cc3ccccc3cc2-c2c(-c3cccc(-c4cc(-c5cccc(-c6ccc7oc8c9ccccc9ccc8c7c6)c5)nc(-c5ccccc5)n4)c3)cccc21 ; CC1(C)c2cc3ccccc3cc2-c2c(-c3cccc(-c4cc(-c5ccccc5-c5ccc6oc7c8ccccc8ccc7c6c5)nc(-c5ccccc5)n4)c3)cccc21 ; CC1(C)c2cc3ccccc3cc2-c2c(-c3cccc(-c4cc(-c5ccc(-c6ccc7oc8cc9ccccc9cc8c7c6)cc5)nc(-c5ccccc5)n4)c3)cccc21]

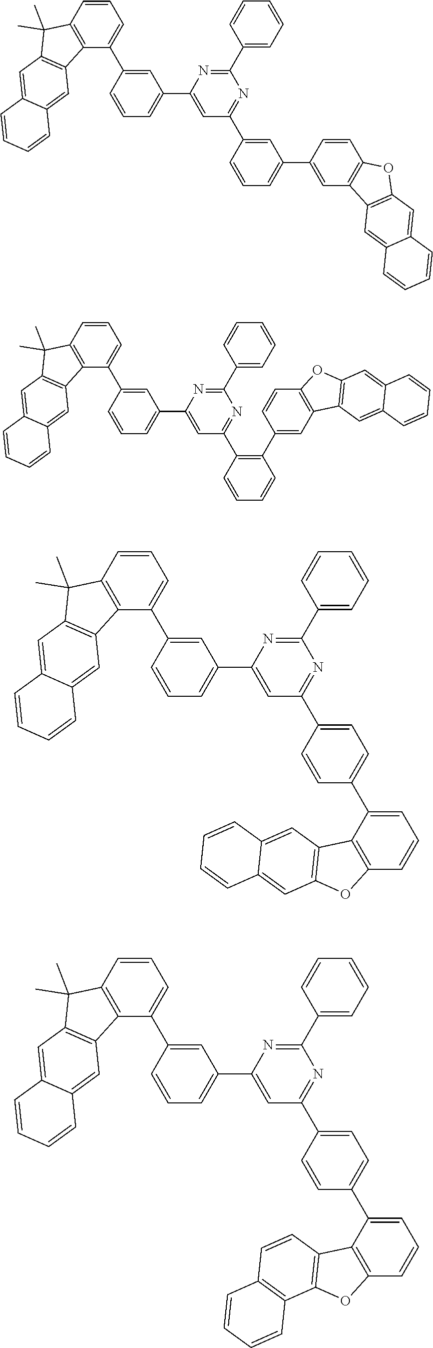 [SMILES: CC1(C)c2cc3ccccc3cc2-c2c(-c3cccc(-c4cc(-c5cccc(-c6ccc7oc8cc9ccccc9cc8c7c6)c5)nc(-c5ccccc5)n4)c3)cccc21 ; CC1(C)c2cc3ccccc3cc2-c2c(-c3cccc(-c4cc(-c5ccccc5-c5ccc6oc7cc8ccccc8cc7c6c5)nc(-c5ccccc5)n4)c3)cccc21 ; CC1(C)c2cc3ccccc3cc2-c2c(-c3cccc(-c4cc(-c5ccc(-c6cccc7oc8cc9ccccc9cc8c67)cc5)nc(-c5ccccc5)n4)c3)cccc21 ; CC1(C)c2cc3ccccc3cc2-c2c(-c3cccc(-c4cc(-c5ccc(-c6cccc7oc8c9ccccc9ccc8c67)cc5)nc(-c5ccccc5)n4)c3)cccc21]

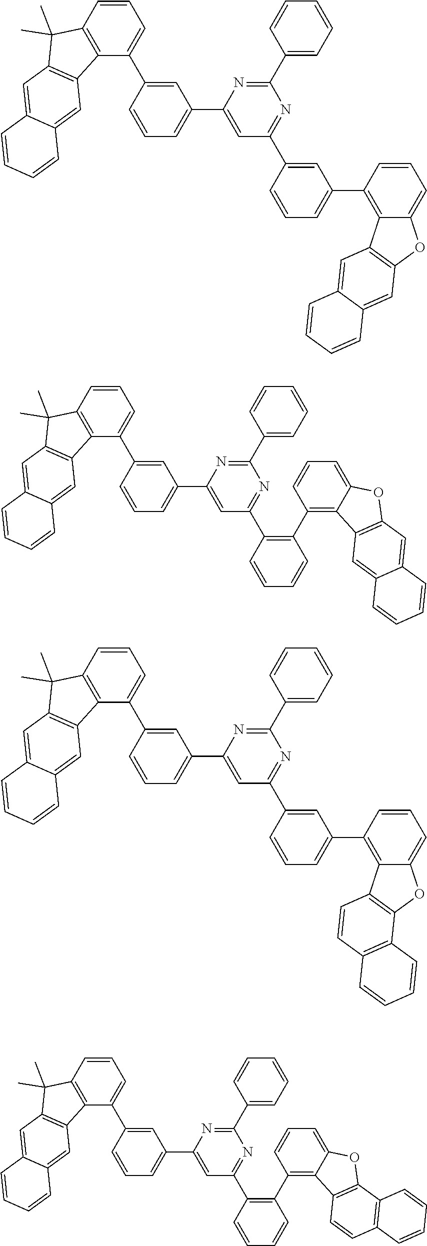 [SMILES: CC1(C)c2cc3ccccc3cc2-c2c(-c3cccc(-c4cc(-c5cccc(-c6cccc7oc8cc9ccccc9cc8c67)c5)nc(-c5ccccc5)n4)c3)cccc21 ; CC1(C)c2cc3ccccc3cc2-c2c(-c3cccc(-c4cc(-c5ccccc5-c5cccc6oc7cc8ccccc8cc7c56)nc(-c5ccccc5)n4)c3)cccc21 ; CC1(C)c2cc3ccccc3cc2-c2c(-c3cccc(-c4cc(-c5cccc(-c6cccc7oc8c9ccccc9ccc8c67)c5)nc(-c5ccccc5)n4)c3)cccc21 ; CC1(C)c2cc3ccccc3cc2-c2c(-c3cccc(-c4cc(-c5ccccc5-c5cccc6oc7c8ccccc8ccc7c56)nc(-c5ccccc5)n4)c3)cccc21]

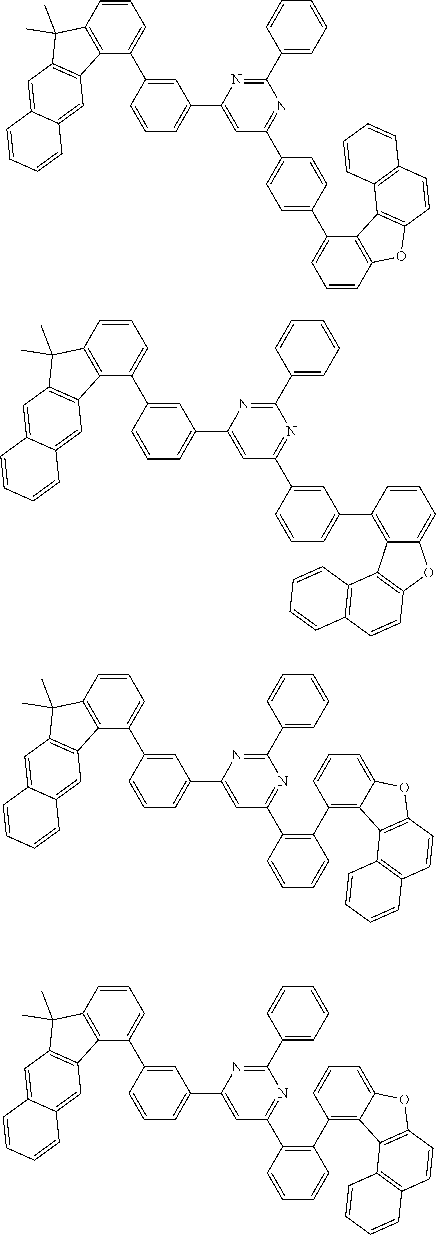

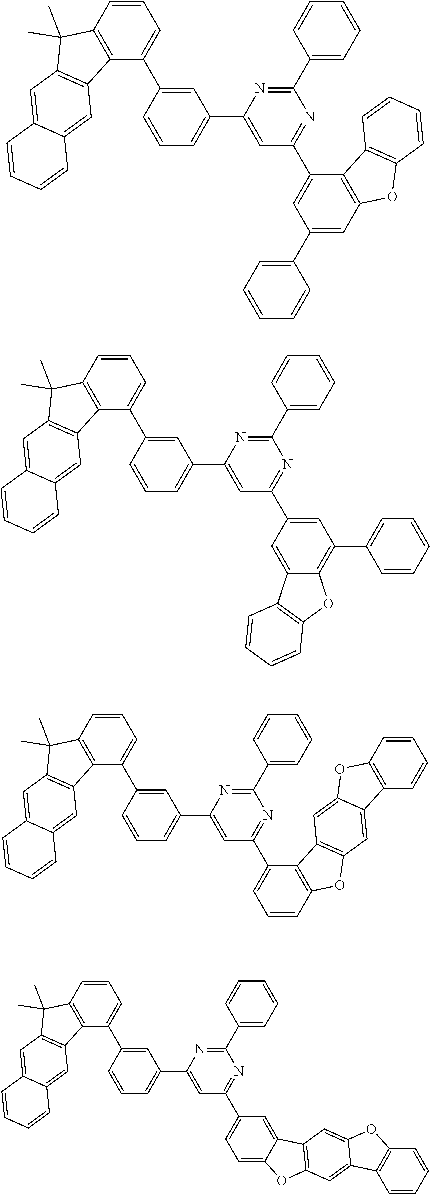

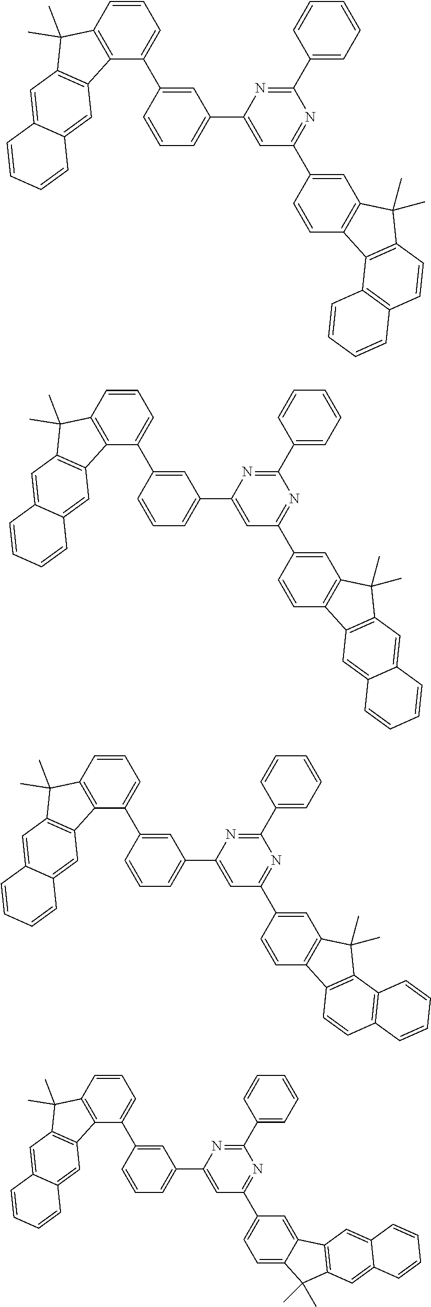 [SMILES: CC1(C)c2cc3ccccc3cc2-c2c(-c3cccc(-c4cc(-c5ccc6c(c5)C(C)(C)c5ccc7ccccc7c5-6)nc(-c5ccccc5)n4)c3)cccc21 ; CC1(C)c2cc(-c3cc(-c4cccc(-c5cccc6c5-c5cc7ccccc7cc5C6(C)C)c4)nc(-c4ccccc4)n3)ccc2-c2cc3ccccc3cc21 ; CC1(C)c2cc3ccccc3cc2-c2c(-c3cccc(-c4cc(-c5ccc6c(c5)C(C)(C)c5c-6ccc6ccccc56)nc(-c5ccccc5)n4)c3)cccc21 ; CC1(C)c2ccc(-c3cc(-c4cccc(-c5cccc6c5-c5cc7ccccc7cc5C6(C)C)c4)nc(-c4ccccc4)n3)cc2-c2cc3ccccc3cc21]

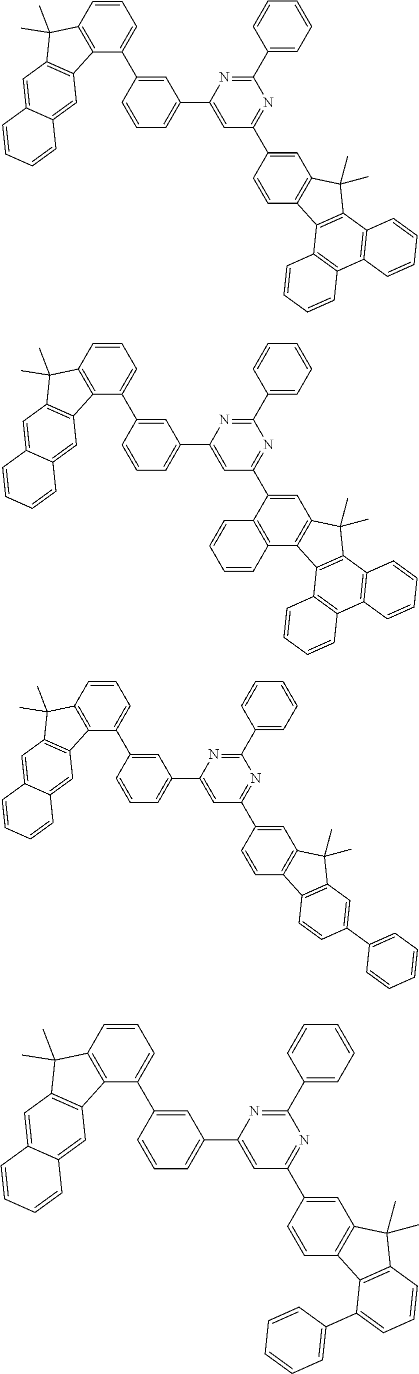

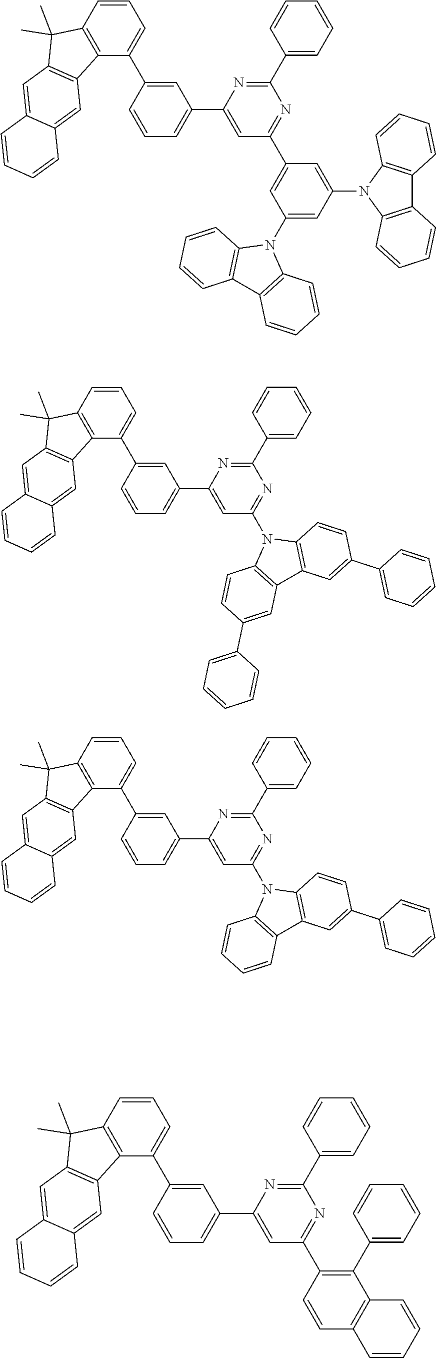

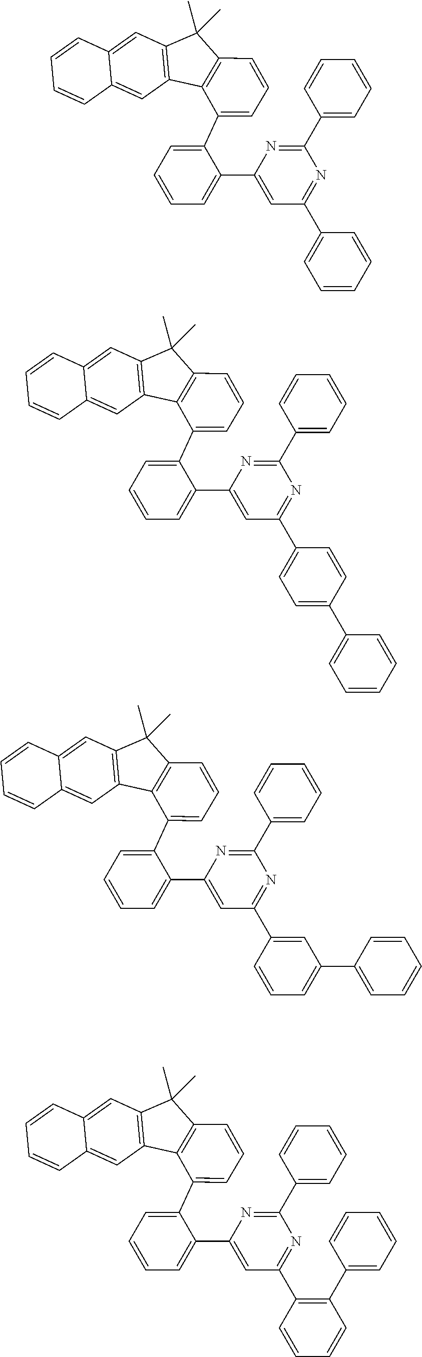 [SMILES: CC1(C)c2cc3ccccc3cc2-c2c(-c3ccccc3-c3cc(-c4ccccc4)nc(-c4ccccc4)n3)cccc21 ; CC1(C)c2cc3ccccc3cc2-c2c(-c3ccccc3-c3cc(-c4ccc(-c5ccccc5)cc4)nc(-c4ccccc4)n3)cccc21 ; CC1(C)c2cc3ccccc3cc2-c2c(-c3ccccc3-c3cc(-c4cccc(-c5ccccc5)c4)nc(-c4ccccc4)n3)cccc21 ; CC1(C)c2cc3ccccc3cc2-c2c(-c3ccccc3-c3cc(-c4ccccc4-c4ccccc4)nc(-c4ccccc4)n3)cccc21]

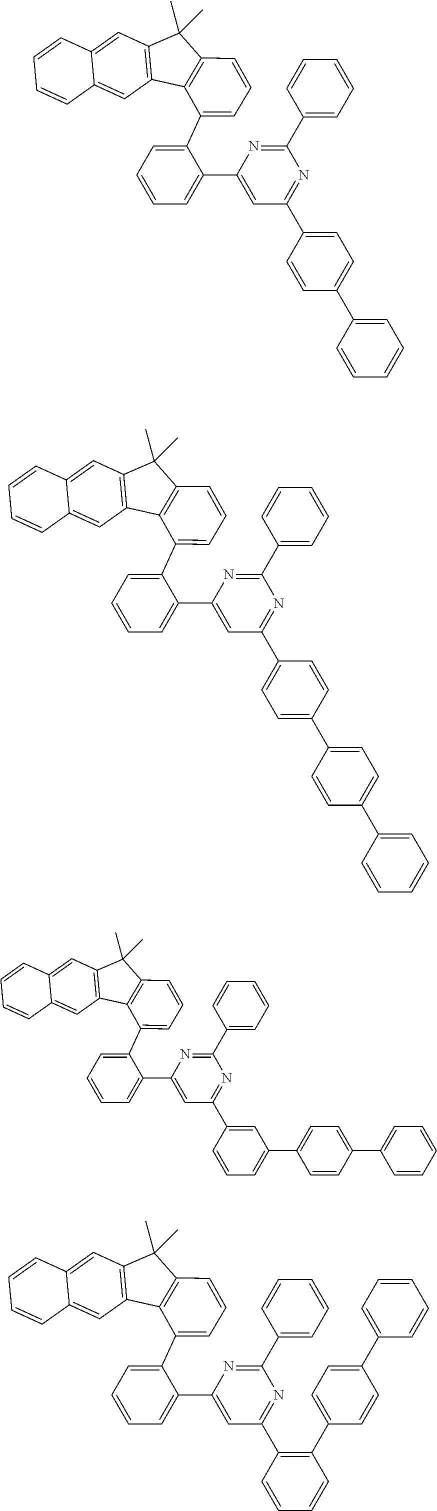 [SMILES: CC1(C)c2cc3ccccc3cc2-c2c(-c3ccccc3-c3cc(-c4ccc(-c5ccccc5)cc4)nc(-c4ccccc4)n3)cccc21 ; CC1(C)c2cc3ccccc3cc2-c2c(-c3ccccc3-c3cc(-c4ccc(-c5ccc(-c6ccccc6)cc5)cc4)nc(-c4ccccc4)n3)cccc21 ; CC1(C)c2cc3ccccc3cc2-c2c(-c3ccccc3-c3cc(-c4cccc(-c5ccc(-c6ccccc6)cc5)c4)nc(-c4ccccc4)n3)cccc21 ; CC1(C)c2cc3ccccc3cc2-c2c(-c3ccccc3-c3cc(-c4ccccc4-c4ccc(-c5ccccc5)cc4)nc(-c4ccccc4)n3)cccc21]

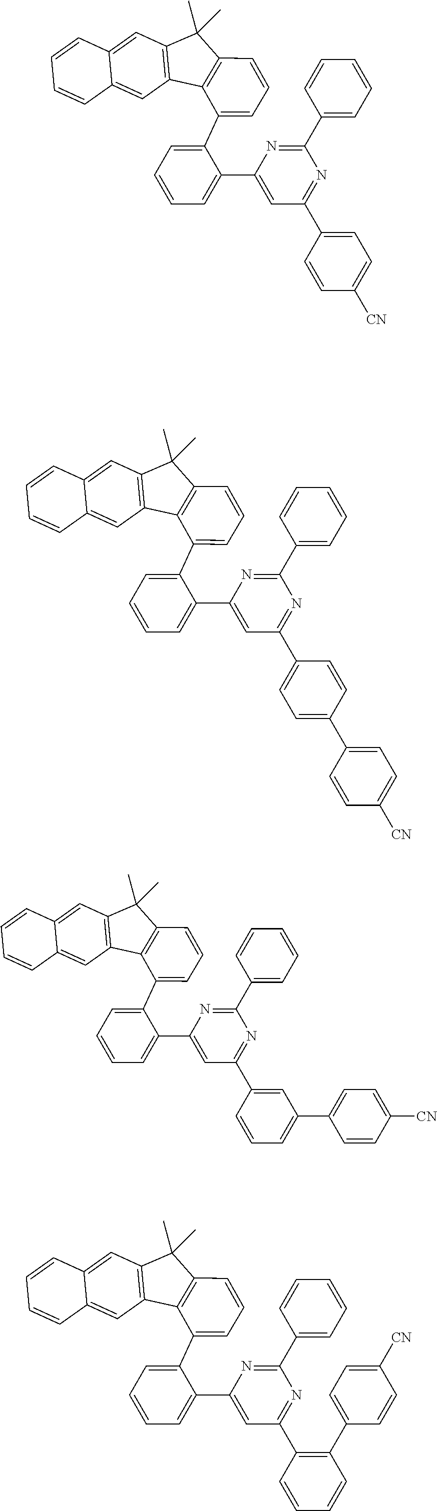 [SMILES: CC1(C)c2cc3ccccc3cc2-c2c(-c3ccccc3-c3cc(-c4ccc(C#N)cc4)nc(-c4ccccc4)n3)cccc21 ; CC1(C)c2cc3ccccc3cc2-c2c(-c3ccccc3-c3cc(-c4ccc(-c5ccc(C#N)cc5)cc4)nc(-c4ccccc4)n3)cccc21 ; CC1(C)c2cc3ccccc3cc2-c2c(-c3ccccc3-c3cc(-c4cccc(-c5ccc(C#N)cc5)c4)nc(-c4ccccc4)n3)cccc21 ; CC1(C)c2cc3ccccc3cc2-c2c(-c3ccccc3-c3cc(-c4ccccc4-c4ccc(C#N)cc4)nc(-c4ccccc4)n3)cccc21]

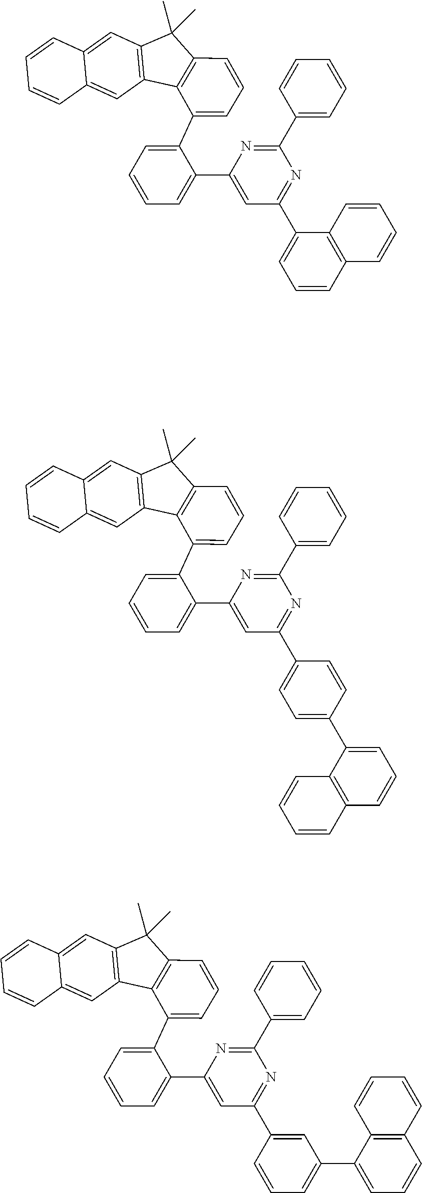 [SMILES: CC1(C)c2cc3ccccc3cc2-c2c(-c3ccccc3-c3cc(-c4cccc5ccccc45)nc(-c4ccccc4)n3)cccc21 ; CC1(C)c2cc3ccccc3cc2-c2c(-c3ccccc3-c3cc(-c4ccc(-c5cccc6ccccc56)cc4)nc(-c4ccccc4)n3)cccc21 ; CC1(C)c2cc3ccccc3cc2-c2c(-c3ccccc3-c3cc(-c4cccc(-c5cccc6ccccc56)c4)nc(-c4ccccc4)n3)cccc21]

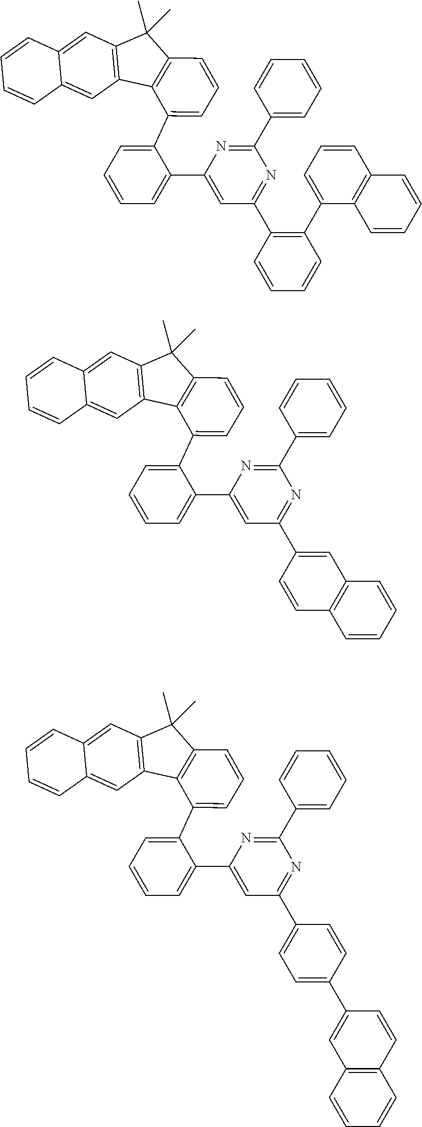 [SMILES: CC1(C)c2cc3ccccc3cc2-c2c(-c3ccccc3-c3cc(-c4ccccc4-c4cccc5ccccc45)nc(-c4ccccc4)n3)cccc21 ; CC1(C)c2cc3ccccc3cc2-c2c(-c3ccccc3-c3cc(-c4ccc5ccccc5c4)nc(-c4ccccc4)n3)cccc21 ; CC1(C)c2cc3ccccc3cc2-c2c(-c3ccccc3-c3cc(-c4ccc(-c5ccc6ccccc6c5)cc4)nc(-c4ccccc4)n3)cccc21]

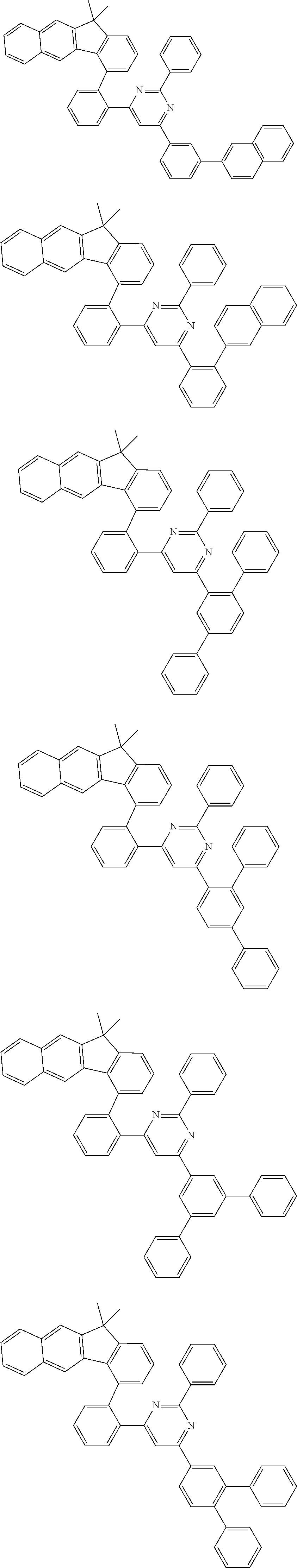

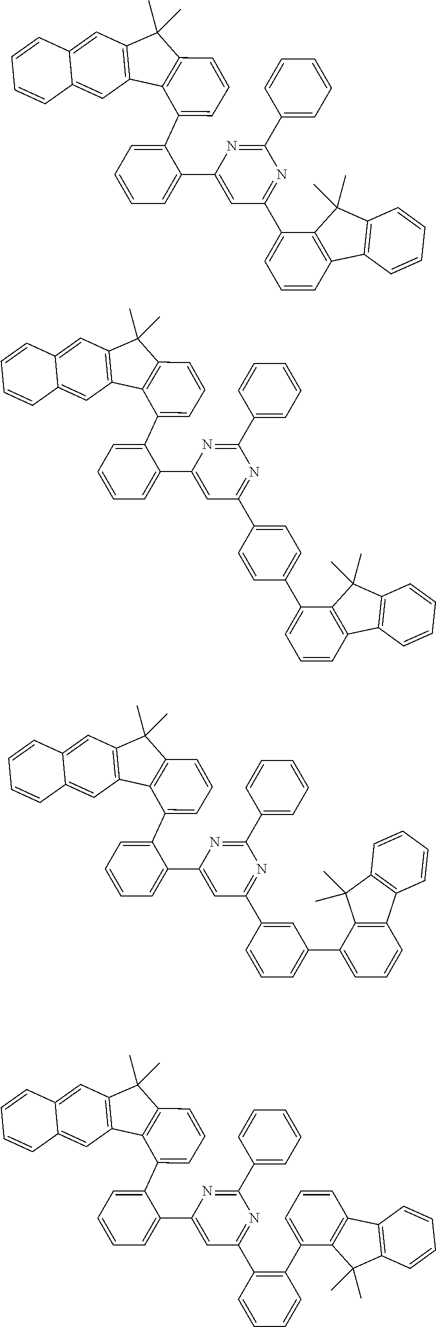 [SMILES: CC1(C)c2cc3ccccc3cc2-c2c(-c3ccccc3-c3cc(-c4cccc5c4C(C)(C)c4ccccc4-5)nc(-c4ccccc4)n3)cccc21 ; CC1(C)c2cc3ccccc3cc2-c2c(-c3ccccc3-c3cc(-c4ccc(-c5cccc6c5C(C)(C)c5ccccc5-6)cc4)nc(-c4ccccc4)n3)cccc21 ; CC1(C)c2cc3ccccc3cc2-c2c(-c3ccccc3-c3cc(-c4cccc(-c5cccc6c5C(C)(C)c5ccccc5-6)c4)nc(-c4ccccc4)n3)cccc21 ; CC1(C)c2cc3ccccc3cc2-c2c(-c3ccccc3-c3cc(-c4ccccc4-c4cccc5c4C(C)(C)c4ccccc4-5)nc(-c4ccccc4)n3)cccc21]

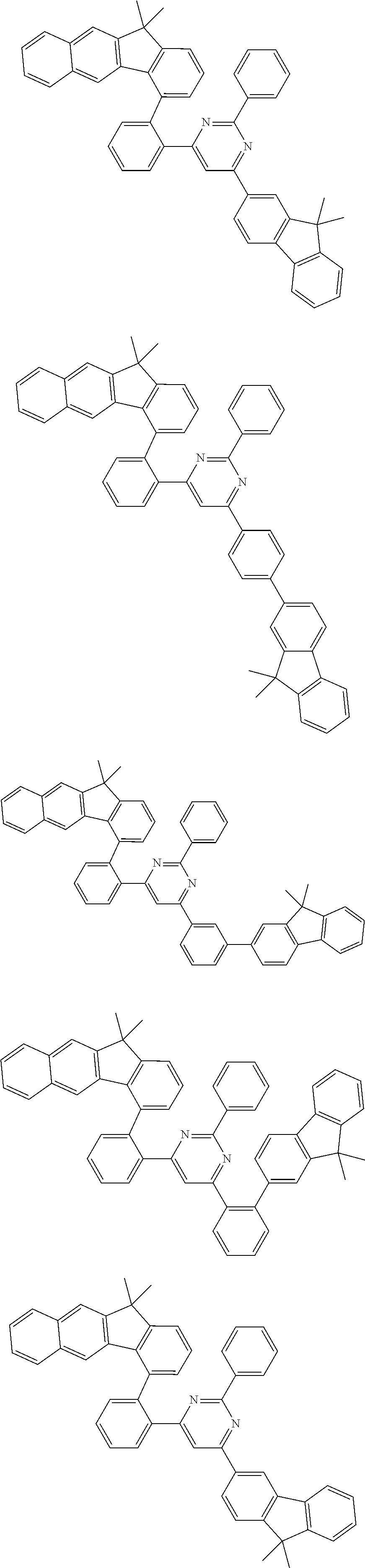 [SMILES: CC1(C)c2ccccc2-c2ccc(-c3cc(-c4ccccc4-c4cccc5c4-c4cc6ccccc6cc4C5(C)C)nc(-c4ccccc4)n3)cc21 ; CC1(C)c2ccccc2-c2ccc(-c3ccc(-c4cc(-c5ccccc5-c5cccc6c5-c5cc7ccccc7cc5C6(C)C)nc(-c5ccccc5)n4)cc3)cc21 ; CC1(C)c2ccccc2-c2ccc(-c3cccc(-c4cc(-c5ccccc5-c5cccc6c5-c5cc7ccccc7cc5C6(C)C)nc(-c5ccccc5)n4)c3)cc21 ; CC1(C)c2ccccc2-c2ccc(-c3ccccc3-c3cc(-c4ccccc4-c4cccc5c4-c4cc6ccccc6cc4C5(C)C)nc(-c4ccccc4)n3)cc21 ; CC1(C)c2ccccc2-c2cc(-c3cc(-c4ccccc4-c4cccc5c4-c4cc6ccccc6cc4C5(C)C)nc(-c4ccccc4)n3)ccc21]

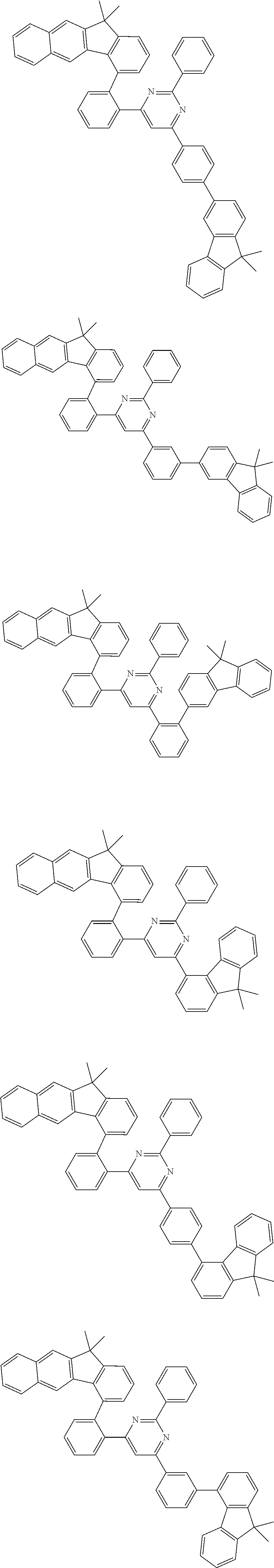

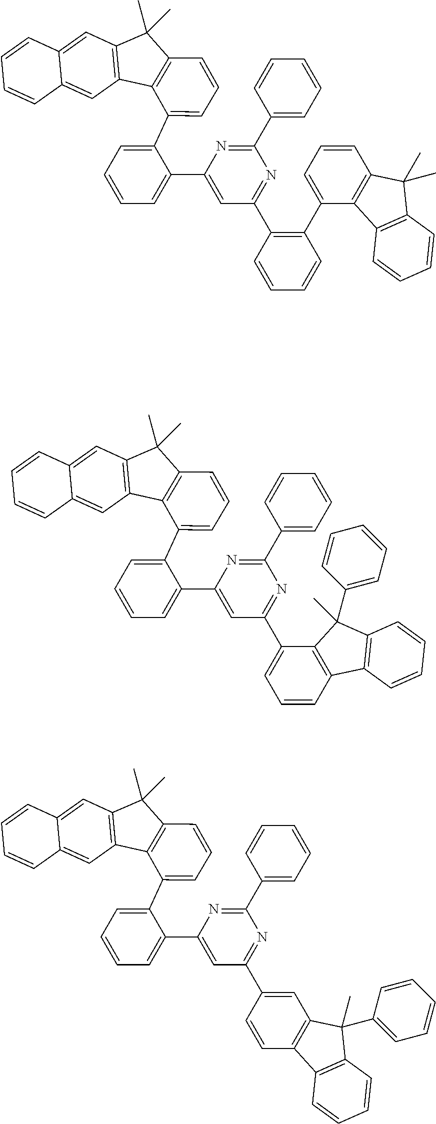 [SMILES: CC1(C)c2ccccc2-c2c(-c3ccccc3-c3cc(-c4ccccc4-c4cccc5c4-c4cc6ccccc6cc4C5(C)C)nc(-c4ccccc4)n3)cccc21 ; CC1(C)c2cc3ccccc3cc2-c2c(-c3ccccc3-c3cc(-c4cccc5c4C(C)(c4ccccc4)c4ccccc4-5)nc(-c4ccccc4)n3)cccc21 ; CC1(C)c2cc3ccccc3cc2-c2c(-c3ccccc3-c3cc(-c4ccc5c(c4)C(C)(c4ccccc4)c4ccccc4-5)nc(-c4ccccc4)n3)cccc21]

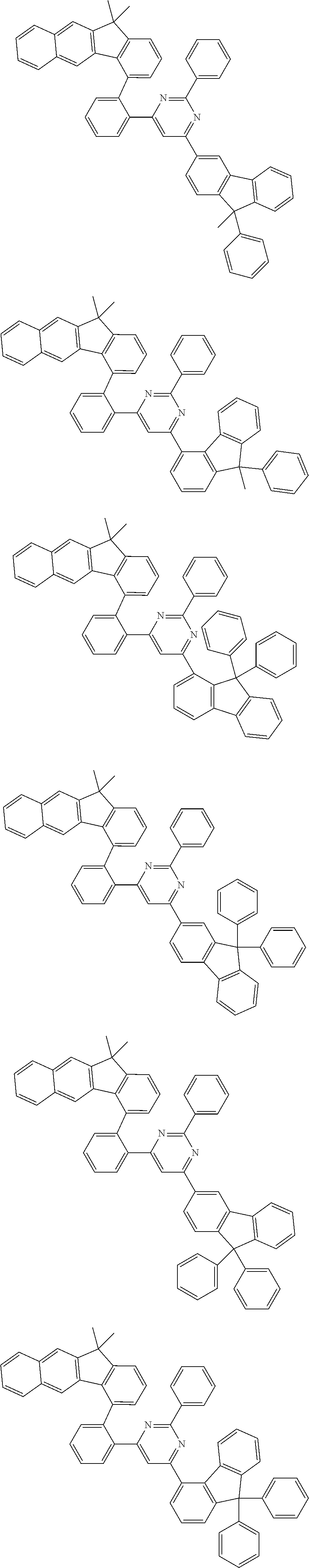

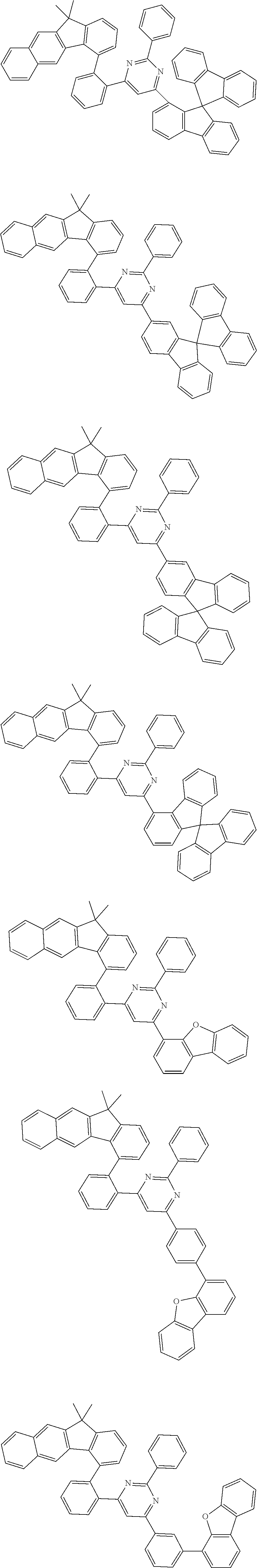

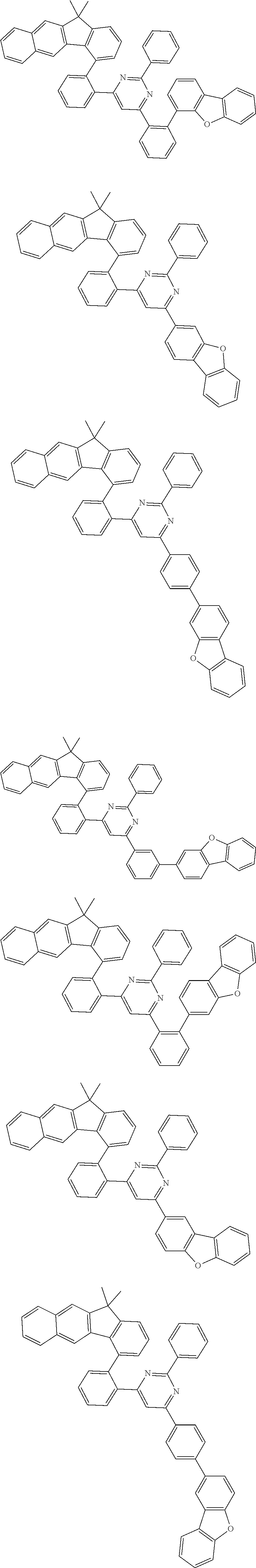

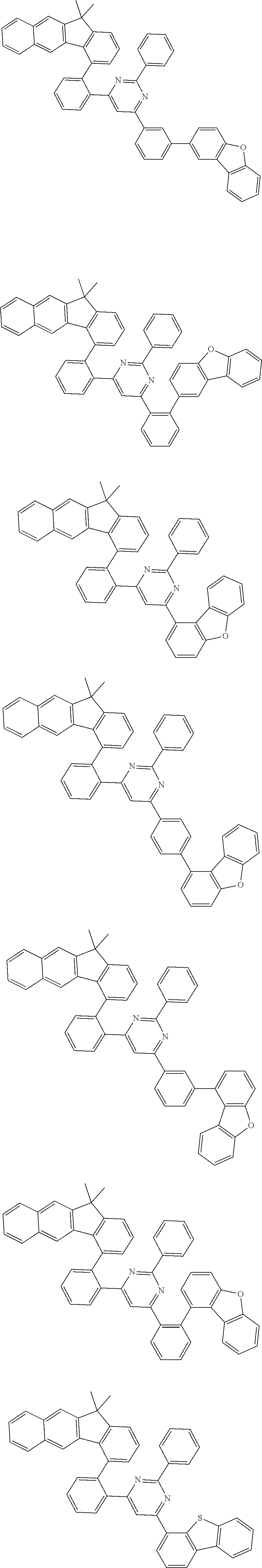

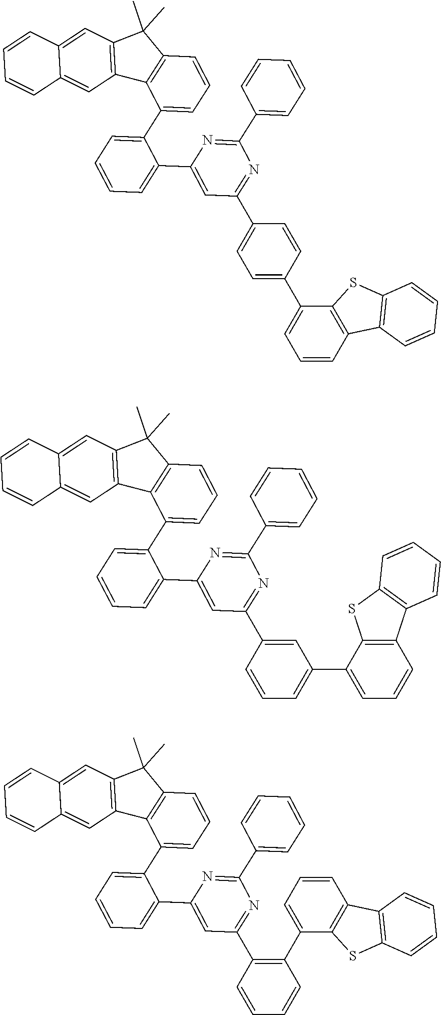 [SMILES: CC1(C)c2cc3ccccc3cc2-c2c(-c3ccccc3-c3cc(-c4ccc(-c5cccc6c5sc5ccccc56)cc4)nc(-c4ccccc4)n3)cccc21 ; CC1(C)c2cc3ccccc3cc2-c2c(-c3ccccc3-c3cc(-c4cccc(-c5cccc6c5sc5ccccc56)c4)nc(-c4ccccc4)n3)cccc21 ; CC1(C)c2cc3ccccc3cc2-c2c(-c3ccccc3-c3cc(-c4ccccc4-c4cccc5c4sc4ccccc45)nc(-c4ccccc4)n3)cccc21]

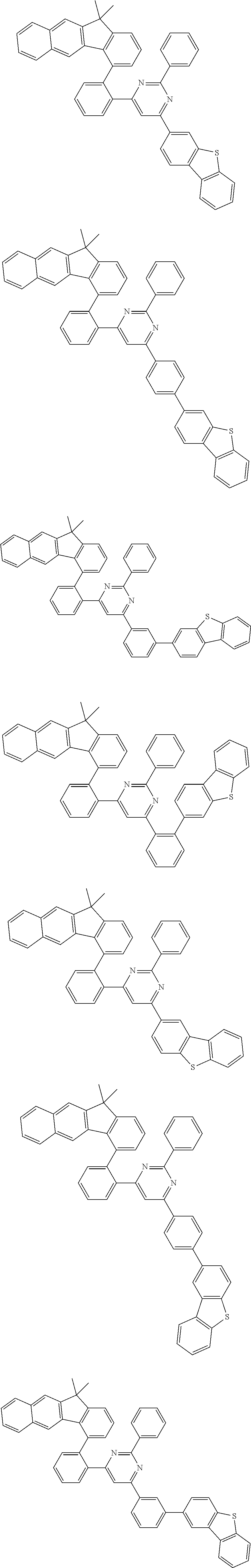

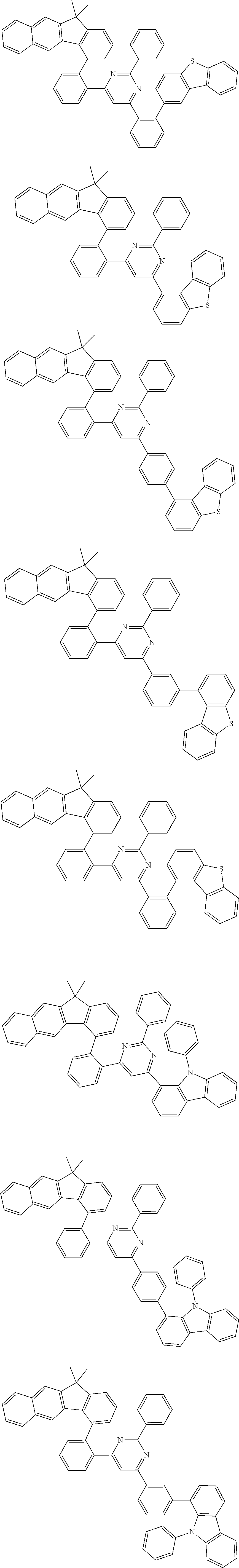

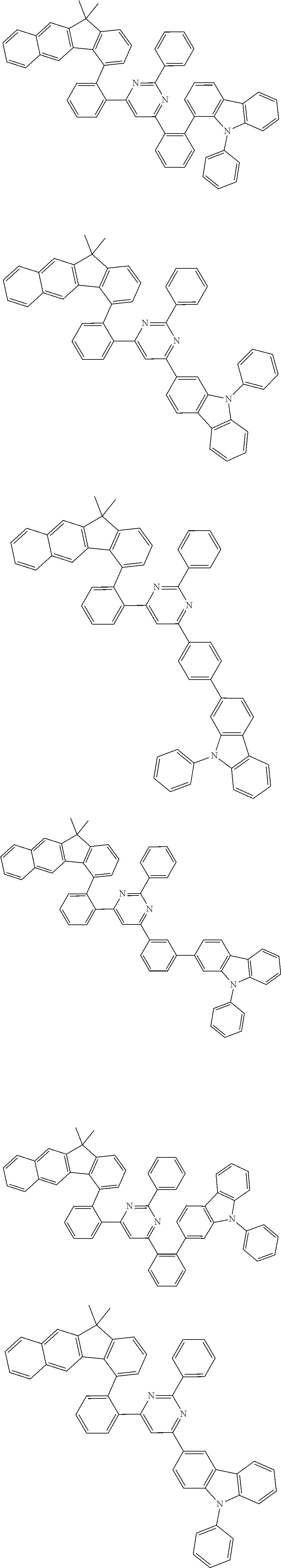

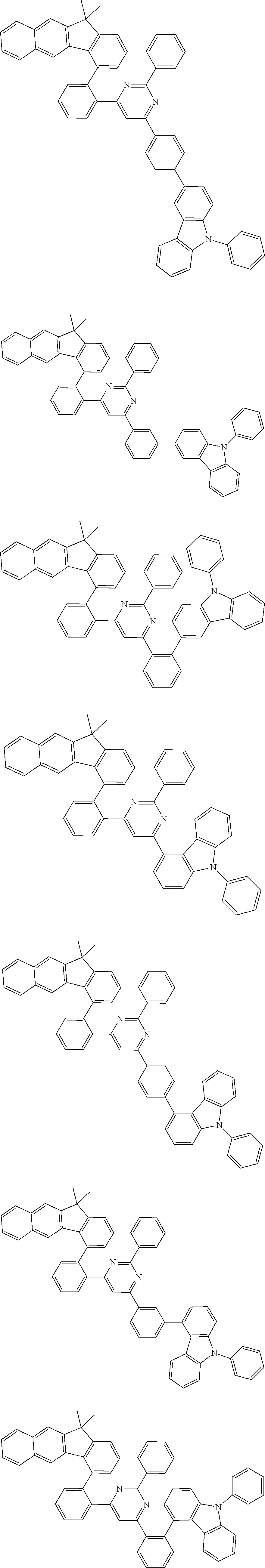

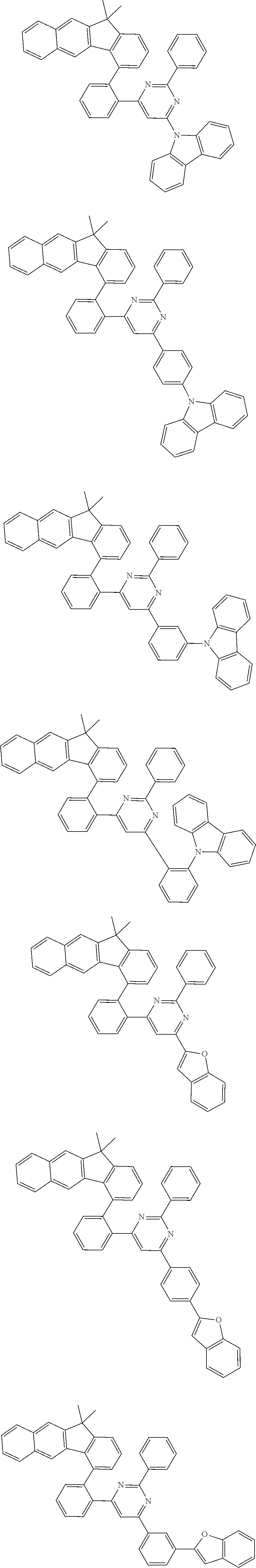

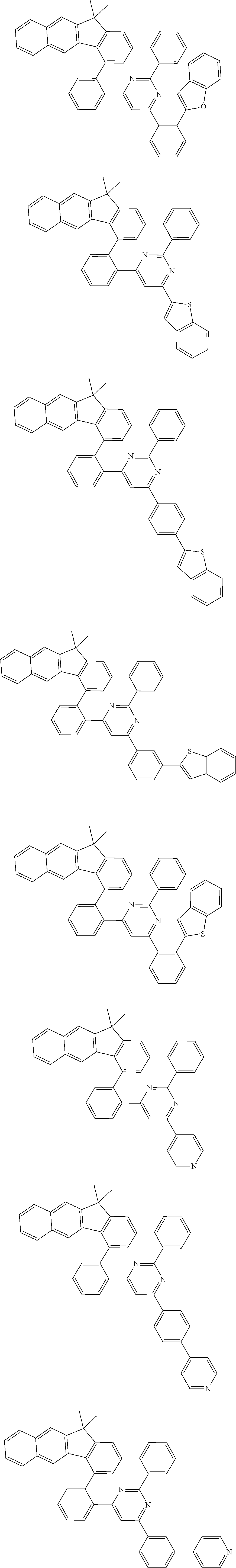

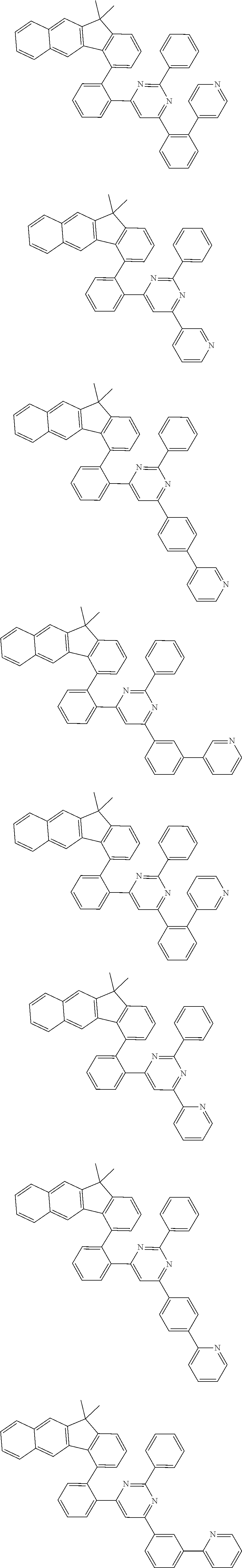

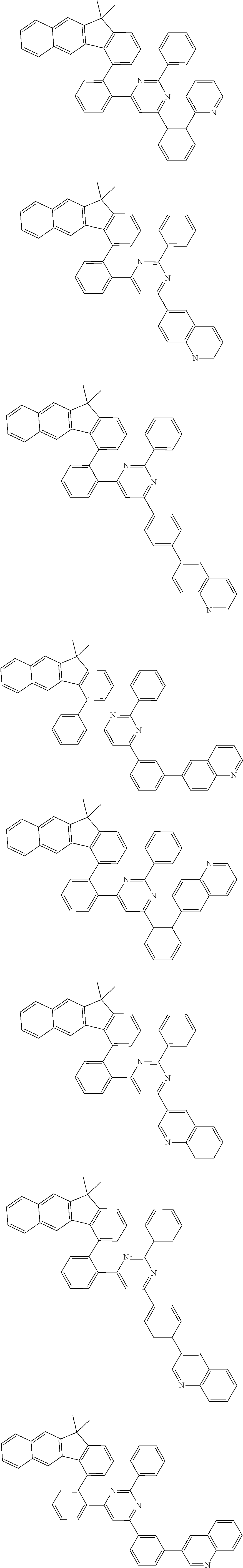

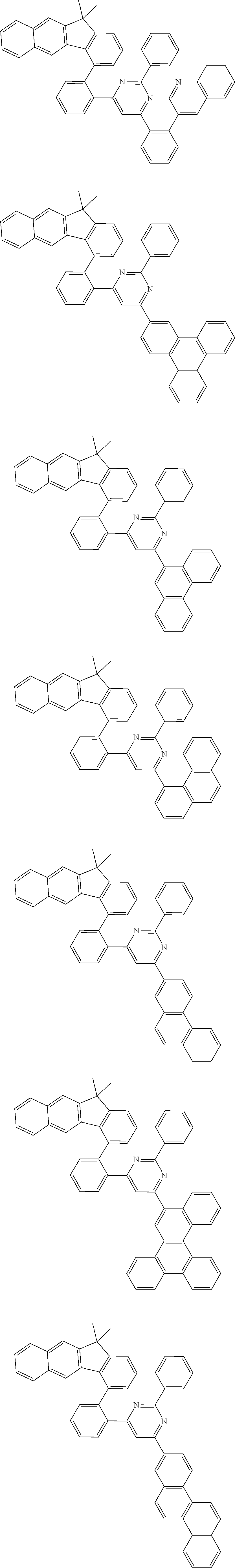

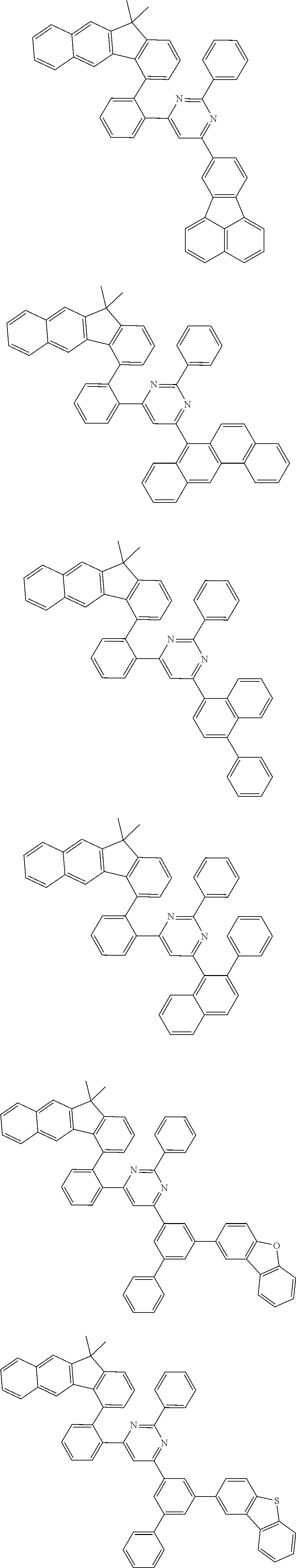

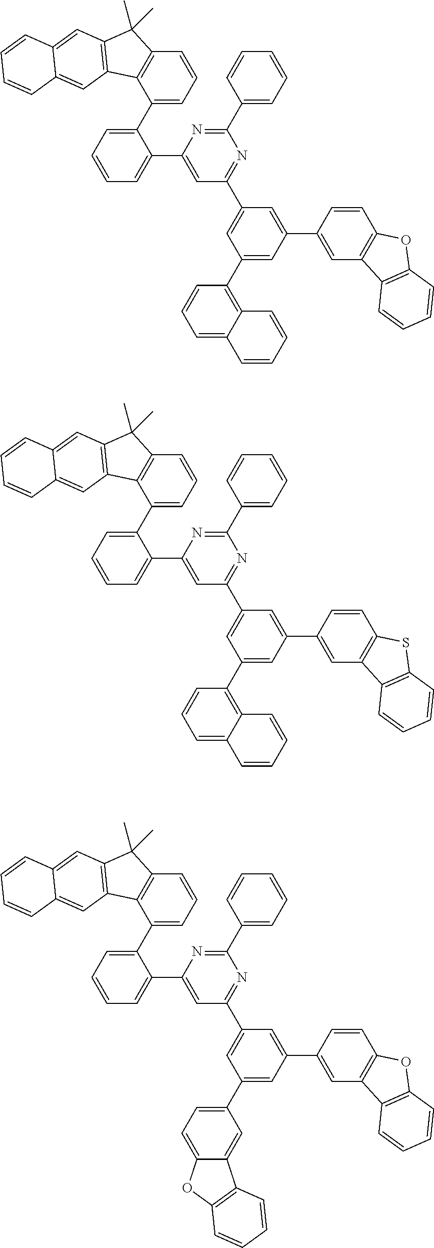 [SMILES: CC1(C)c2cc3ccccc3cc2-c2c(-c3ccccc3-c3cc(-c4cc(-c5ccc6oc7ccccc7c6c5)cc(-c5cccc6ccccc56)c4)nc(-c4ccccc4)n3)cccc21 ; CC1(C)c2cc3ccccc3cc2-c2c(-c3ccccc3-c3cc(-c4cc(-c5ccc6sc7ccccc7c6c5)cc(-c5cccc6ccccc56)c4)nc(-c4ccccc4)n3)cccc21 ; CC1(C)c2cc3ccccc3cc2-c2c(-c3ccccc3-c3cc(-c4cc(-c5ccc6oc7ccccc7c6c5)cc(-c5ccc6oc7ccccc7c6c5)c4)nc(-c4ccccc4)n3)cccc21]

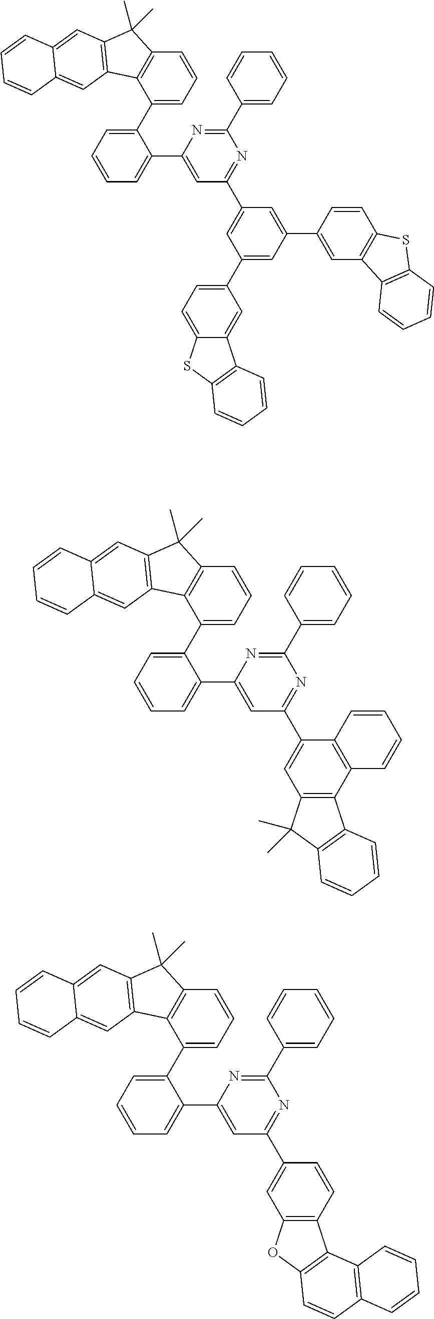 [SMILES: CC1(C)c2cc3ccccc3cc2-c2c(-c3ccccc3-c3cc(-c4cc(-c5ccc6sc7ccccc7c6c5)cc(-c5ccc6sc7ccccc7c6c5)c4)nc(-c4ccccc4)n3)cccc21 ; CC1(C)c2cc3ccccc3cc2-c2c(-c3ccccc3-c3cc(-c4cc5c(c6ccccc46)-c4ccccc4C5(C)C)nc(-c4ccccc4)n3)cccc21 ; CC1(C)c2cc3ccccc3cc2-c2c(-c3ccccc3-c3cc(-c4ccc5c(c4)oc4ccc6ccccc6c45)nc(-c4ccccc4)n3)cccc21]

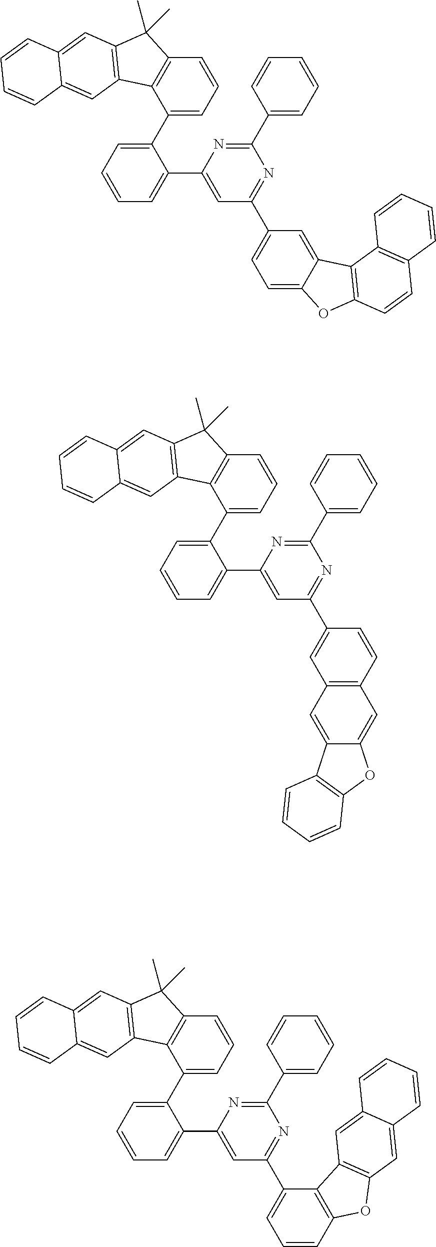 [SMILES: CC1(C)c2cc3ccccc3cc2-c2c(-c3ccccc3-c3cc(-c4ccc5oc6ccc7ccccc7c6c5c4)nc(-c4ccccc4)n3)cccc21 ; CC1(C)c2cc3ccccc3cc2-c2c(-c3ccccc3-c3cc(-c4ccc5cc6oc7ccccc7c6cc5c4)nc(-c4ccccc4)n3)cccc21 ; CC1(C)c2cc3ccccc3cc2-c2c(-c3ccccc3-c3cc(-c4cccc5oc6cc7ccccc7cc6c45)nc(-c4ccccc4)n3)cccc21]

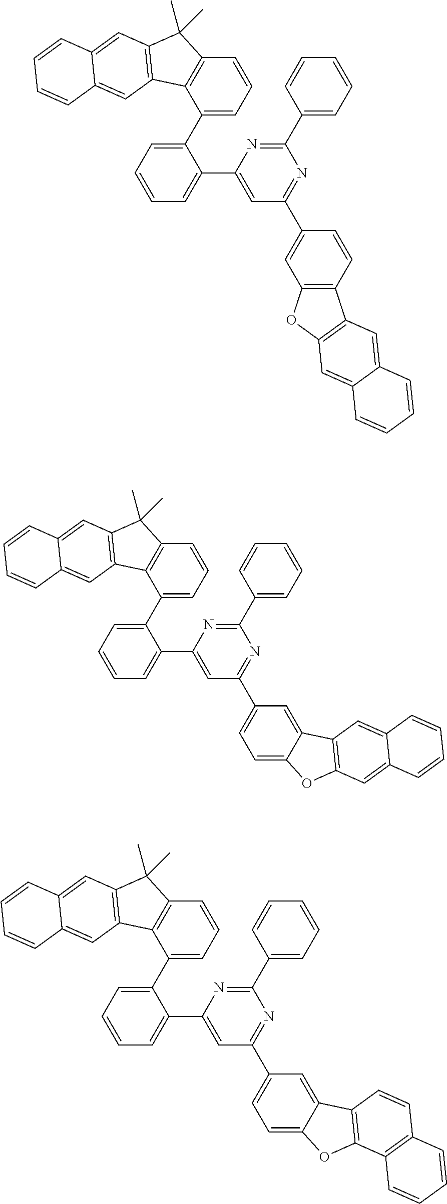 [SMILES: CC1(C)c2cc3ccccc3cc2-c2c(-c3ccccc3-c3cc(-c4ccc5c(c4)oc4cc6ccccc6cc45)nc(-c4ccccc4)n3)cccc21 ; CC1(C)c2cc3ccccc3cc2-c2c(-c3ccccc3-c3cc(-c4ccc5oc6cc7ccccc7cc6c5c4)nc(-c4ccccc4)n3)cccc21 ; CC1(C)c2cc3ccccc3cc2-c2c(-c3ccccc3-c3cc(-c4ccc5oc6c7ccccc7ccc6c5c4)nc(-c4ccccc4)n3)cccc21]

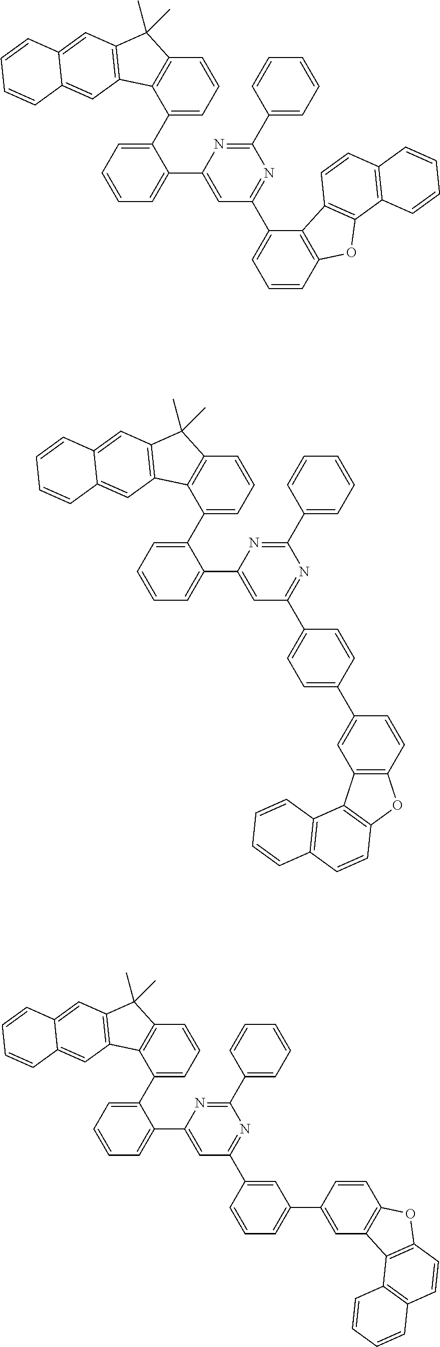 [SMILES: CC1(C)c2cc3ccccc3cc2-c2c(-c3ccccc3-c3cc(-c4cccc5oc6c7ccccc7ccc6c45)nc(-c4ccccc4)n3)cccc21 ; CC1(C)c2cc3ccccc3cc2-c2c(-c3ccccc3-c3cc(-c4ccc(-c5ccc6oc7ccc8ccccc8c7c6c5)cc4)nc(-c4ccccc4)n3)cccc21 ; CC1(C)c2cc3ccccc3cc2-c2c(-c3ccccc3-c3cc(-c4cccc(-c5ccc6oc7ccc8ccccc8c7c6c5)c4)nc(-c4ccccc4)n3)cccc21]

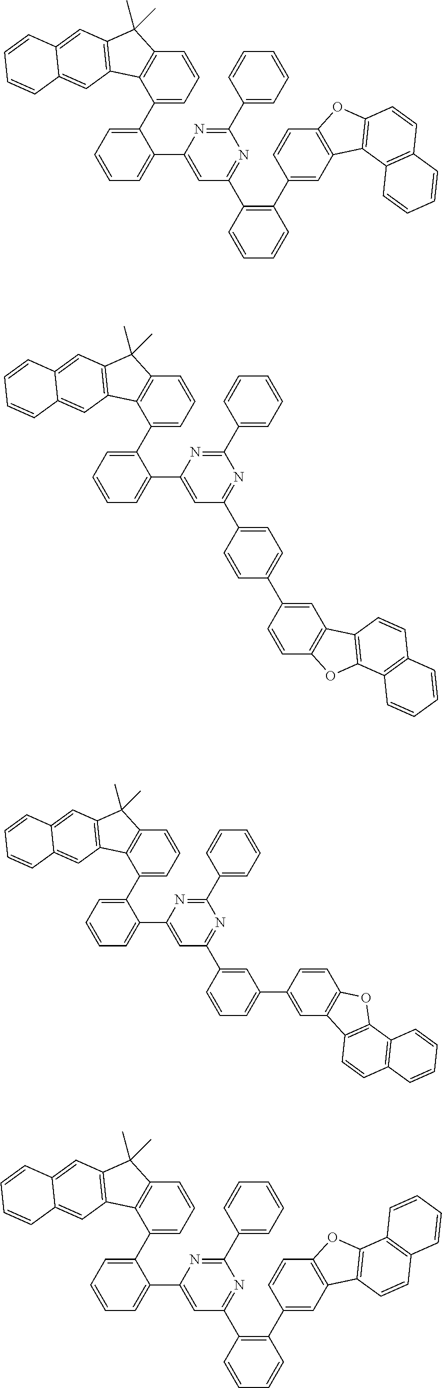 [SMILES: CC1(C)c2cc3ccccc3cc2-c2c(-c3ccccc3-c3cc(-c4ccccc4-c4ccc5oc6ccc7ccccc7c6c5c4)nc(-c4ccccc4)n3)cccc21 ; CC1(C)c2cc3ccccc3cc2-c2c(-c3ccccc3-c3cc(-c4ccc(-c5ccc6oc7c8ccccc8ccc7c6c5)cc4)nc(-c4ccccc4)n3)cccc21 ; CC1(C)c2cc3ccccc3cc2-c2c(-c3ccccc3-c3cc(-c4cccc(-c5ccc6oc7c8ccccc8ccc7c6c5)c4)nc(-c4ccccc4)n3)cccc21 ; CC1(C)c2cc3ccccc3cc2-c2c(-c3ccccc3-c3cc(-c4ccccc4-c4ccc5oc6c7ccccc7ccc6c5c4)nc(-c4ccccc4)n3)cccc21]

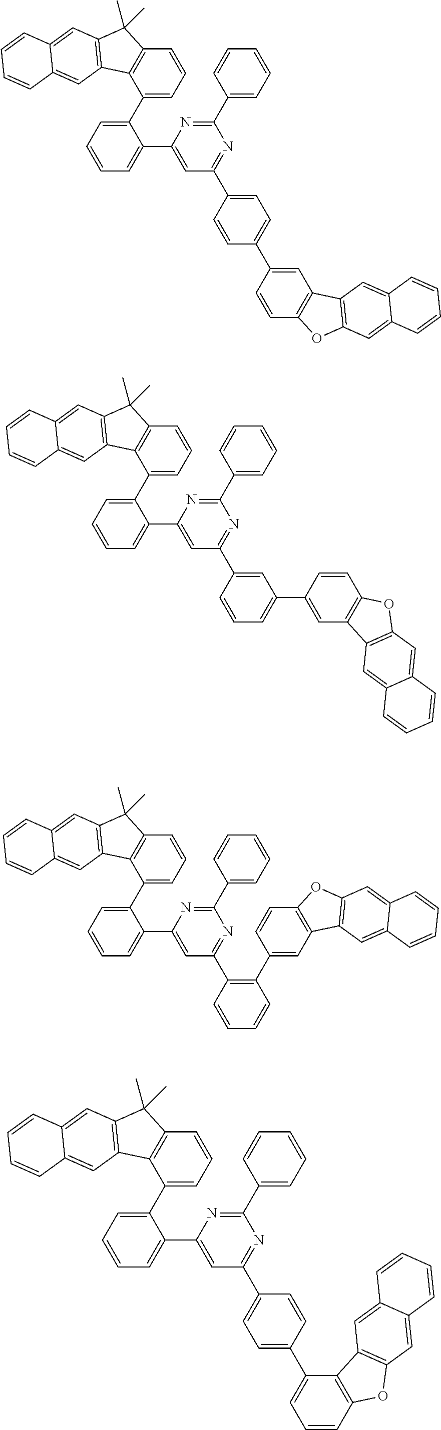 [SMILES: CC1(C)c2cc3ccccc3cc2-c2c(-c3ccccc3-c3cc(-c4ccc(-c5ccc6oc7cc8ccccc8cc7c6c5)cc4)nc(-c4ccccc4)n3)cccc21 ; CC1(C)c2cc3ccccc3cc2-c2c(-c3ccccc3-c3cc(-c4cccc(-c5ccc6oc7cc8ccccc8cc7c6c5)c4)nc(-c4ccccc4)n3)cccc21 ; CC1(C)c2cc3ccccc3cc2-c2c(-c3ccccc3-c3cc(-c4ccccc4-c4ccc5oc6cc7ccccc7cc6c5c4)nc(-c4ccccc4)n3)cccc21 ; CC1(C)c2cc3ccccc3cc2-c2c(-c3ccccc3-c3cc(-c4ccc(-c5cccc6oc7cc8ccccc8cc7c56)cc4)nc(-c4ccccc4)n3)cccc21]

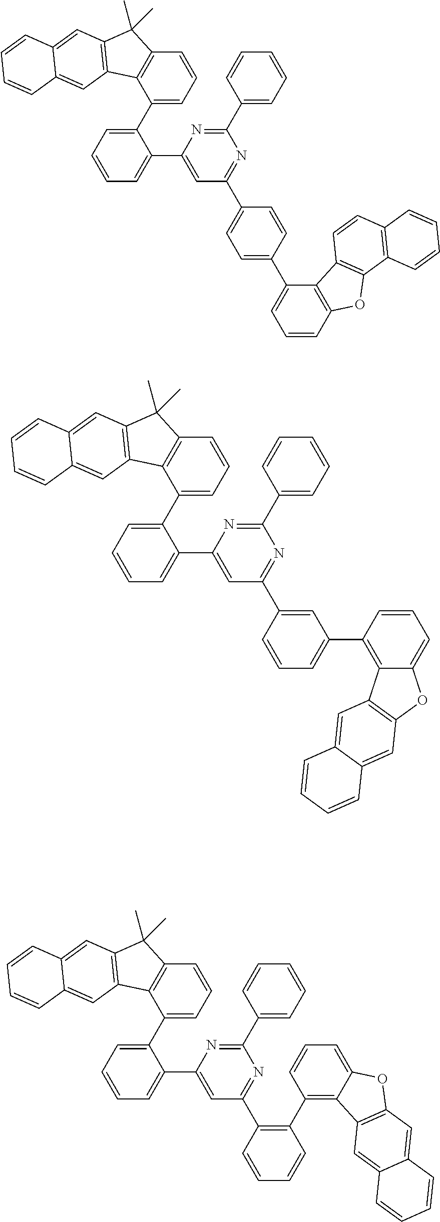 [SMILES: CC1(C)c2cc3ccccc3cc2-c2c(-c3ccccc3-c3cc(-c4ccc(-c5cccc6oc7c8ccccc8ccc7c56)cc4)nc(-c4ccccc4)n3)cccc21 ; CC1(C)c2cc3ccccc3cc2-c2c(-c3ccccc3-c3cc(-c4cccc(-c5cccc6oc7cc8ccccc8cc7c56)c4)nc(-c4ccccc4)n3)cccc21 ; CC1(C)c2cc3ccccc3cc2-c2c(-c3ccccc3-c3cc(-c4ccccc4-c4cccc5oc6cc7ccccc7cc6c45)nc(-c4ccccc4)n3)cccc21]

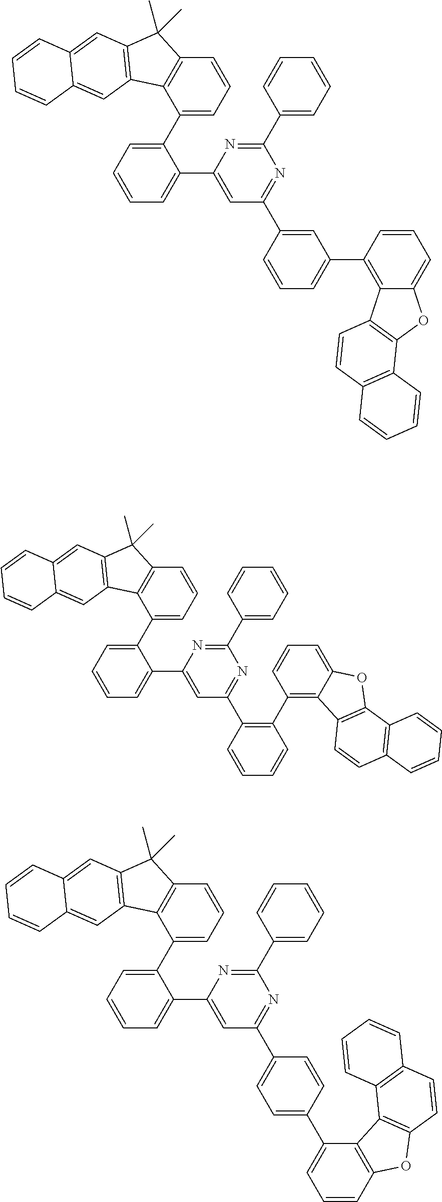 [SMILES: CC1(C)c2cc3ccccc3cc2-c2c(-c3ccccc3-c3cc(-c4cccc(-c5cccc6oc7c8ccccc8ccc7c56)c4)nc(-c4ccccc4)n3)cccc21 ; CC1(C)c2cc3ccccc3cc2-c2c(-c3ccccc3-c3cc(-c4ccccc4-c4cccc5oc6c7ccccc7ccc6c45)nc(-c4ccccc4)n3)cccc21 ; CC1(C)c2cc3ccccc3cc2-c2c(-c3ccccc3-c3cc(-c4ccc(-c5cccc6oc7ccc8ccccc8c7c56)cc4)nc(-c4ccccc4)n3)cccc21]

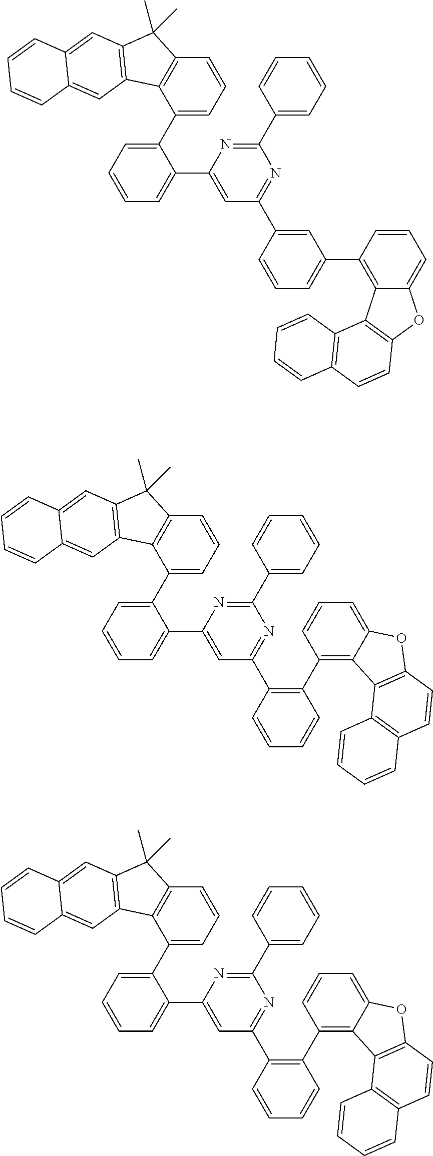 [SMILES: CC1(C)c2cc3ccccc3cc2-c2c(-c3ccccc3-c3cc(-c4cccc(-c5cccc6oc7ccc8ccccc8c7c56)c4)nc(-c4ccccc4)n3)cccc21 ; CC1(C)c2cc3ccccc3cc2-c2c(-c3ccccc3-c3cc(-c4ccccc4-c4cccc5oc6ccc7ccccc7c6c45)nc(-c4ccccc4)n3)cccc21 ; CC1(C)c2cc3ccccc3cc2-c2c(-c3ccccc3-c3cc(-c4ccccc4-c4cccc5oc6ccc7ccccc7c6c45)nc(-c4ccccc4)n3)cccc21]

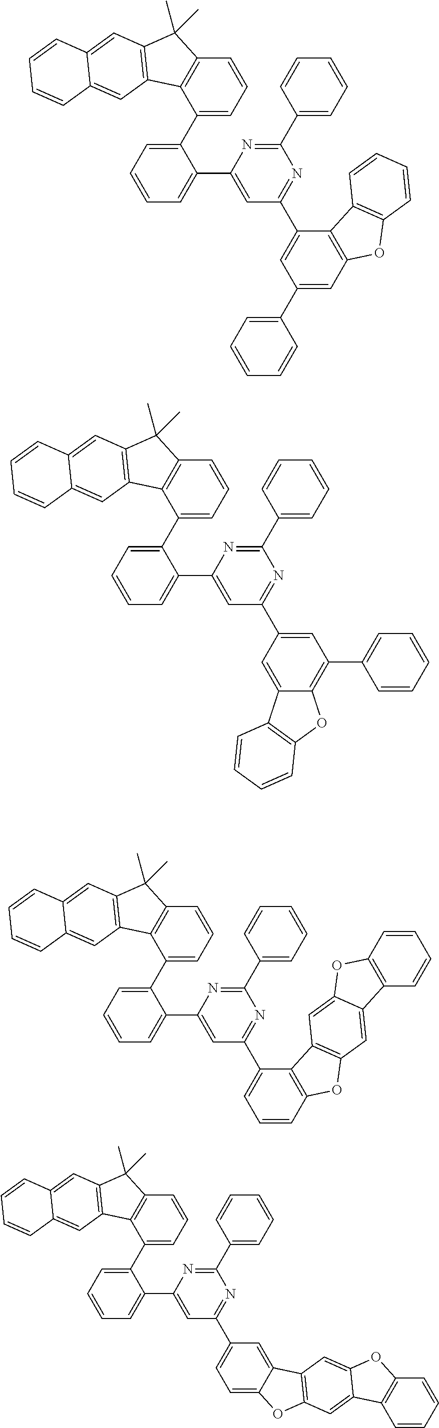 [SMILES: CC1(C)c2cc3ccccc3cc2-c2c(-c3ccccc3-c3cc(-c4cc(-c5ccccc5)cc5oc6ccccc6c45)nc(-c4ccccc4)n3)cccc21 ; CC1(C)c2cc3ccccc3cc2-c2c(-c3ccccc3-c3cc(-c4cc(-c5ccccc5)c5oc6ccccc6c5c4)nc(-c4ccccc4)n3)cccc21 ; CC1(C)c2cc3ccccc3cc2-c2c(-c3ccccc3-c3cc(-c4cccc5oc6cc7c(cc6c45)oc4ccccc47)nc(-c4ccccc4)n3)cccc21 ; CC1(C)c2cc3ccccc3cc2-c2c(-c3ccccc3-c3cc(-c4ccc5oc6cc7c(cc6c5c4)oc4ccccc47)nc(-c4ccccc4)n3)cccc21]

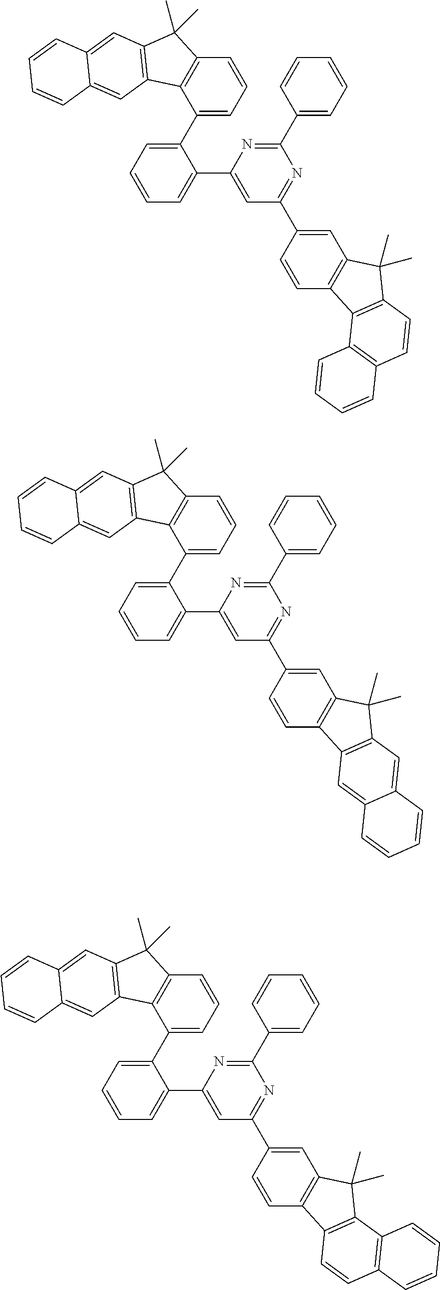 [SMILES: CC1(C)c2cc3ccccc3cc2-c2c(-c3ccccc3-c3cc(-c4ccc5c(c4)C(C)(C)c4ccc6ccccc6c4-5)nc(-c4ccccc4)n3)cccc21 ; CC1(C)c2cc(-c3cc(-c4ccccc4-c4cccc5c4-c4cc6ccccc6cc4C5(C)C)nc(-c4ccccc4)n3)ccc2-c2cc3ccccc3cc21 ; CC1(C)c2cc3ccccc3cc2-c2c(-c3ccccc3-c3cc(-c4ccc5c(c4)C(C)(C)c4c-5ccc5ccccc45)nc(-c4ccccc4)n3)cccc21]

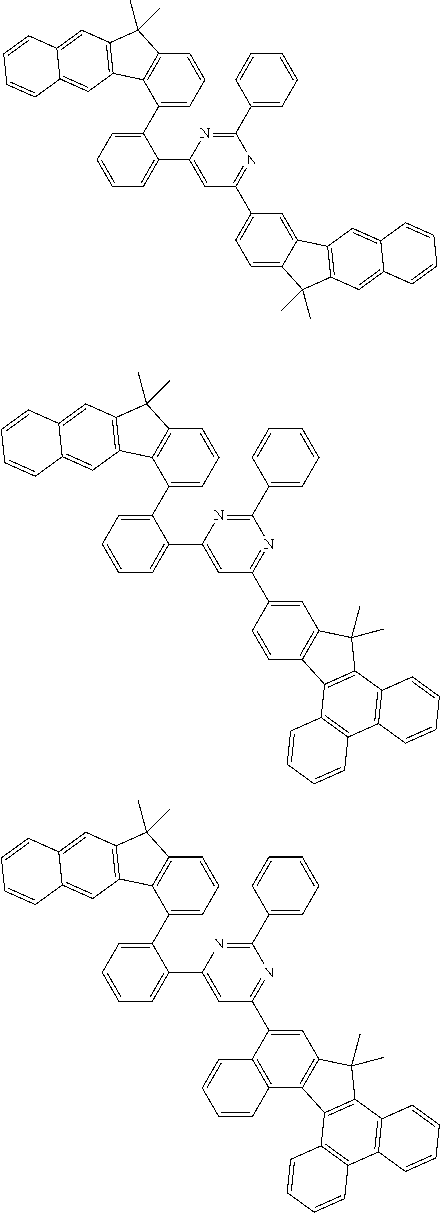 [SMILES: CC1(C)c2ccc(-c3cc(-c4ccccc4-c4cccc5c4-c4cc6ccccc6cc4C5(C)C)nc(-c4ccccc4)n3)cc2-c2cc3ccccc3cc21 ; CC1(C)c2cc3ccccc3cc2-c2c(-c3ccccc3-c3cc(-c4ccc5c(c4)C(C)(C)c4c-5c5ccccc5c5ccccc45)nc(-c4ccccc4)n3)cccc21 ; CC1(C)c2cc3ccccc3cc2-c2c(-c3ccccc3-c3cc(-c4cc5c(c6ccccc46)-c4c(c6ccccc6c6ccccc46)C5(C)C)nc(-c4ccccc4)n3)cccc21]

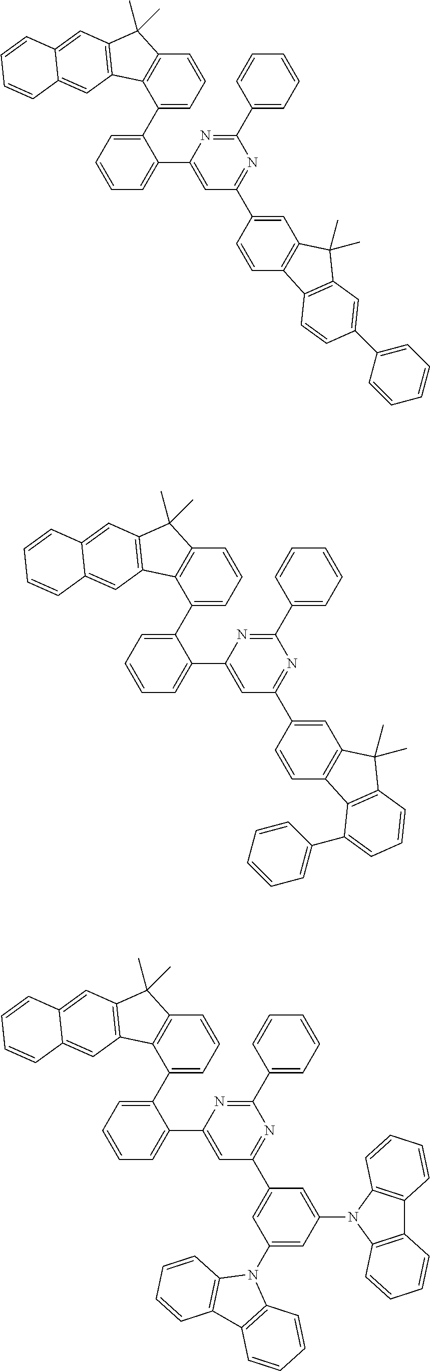 [SMILES: CC1(C)c2cc(-c3ccccc3)ccc2-c2ccc(-c3cc(-c4ccccc4-c4cccc5c4-c4cc6ccccc6cc4C5(C)C)nc(-c4ccccc4)n3)cc21 ; CC1(C)c2cc(-c3cc(-c4ccccc4-c4cccc5c4-c4cc6ccccc6cc4C5(C)C)nc(-c4ccccc4)n3)ccc2-c2c(-c3ccccc3)cccc21 ; CC1(C)c2cc3ccccc3cc2-c2c(-c3ccccc3-c3cc(-c4cc(-n5c6ccccc6c6ccccc65)cc(-n5c6ccccc6c6ccccc65)c4)nc(-c4ccccc4)n3)cccc21]

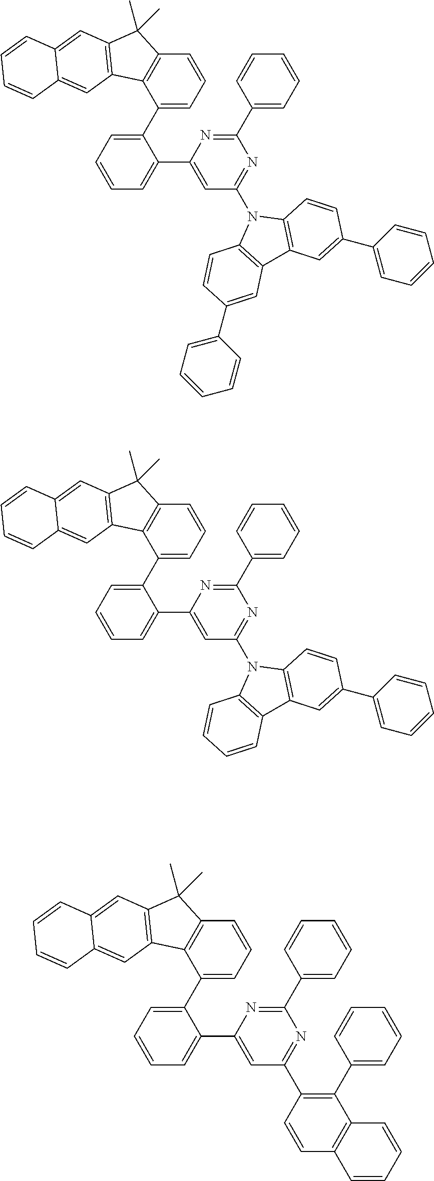 [SMILES: CC1(C)c2cc3ccccc3cc2-c2c(-c3ccccc3-c3cc(-n4c5ccc(-c6ccccc6)cc5c5cc(-c6ccccc6)ccc54)nc(-c4ccccc4)n3)cccc21 ; CC1(C)c2cc3ccccc3cc2-c2c(-c3ccccc3-c3cc(-n4c5ccccc5c5cc(-c6ccccc6)ccc54)nc(-c4ccccc4)n3)cccc21 ; CC1(C)c2cc3ccccc3cc2-c2c(-c3ccccc3-c3cc(-c4ccc5ccccc5c4-c4ccccc4)nc(-c4ccccc4)n3)cccc21]

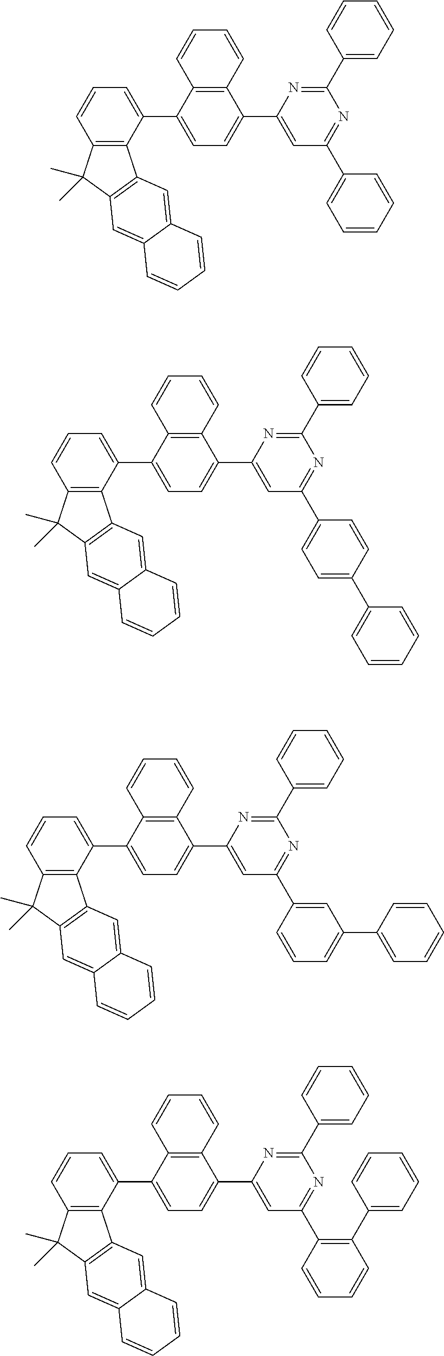 [SMILES: CC1(C)c2cc3ccccc3cc2-c2c(-c3ccc(-c4cc(-c5ccccc5)nc(-c5ccccc5)n4)c4ccccc34)cccc21 ; CC1(C)c2cc3ccccc3cc2-c2c(-c3ccc(-c4cc(-c5ccc(-c6ccccc6)cc5)nc(-c5ccccc5)n4)c4ccccc34)cccc21 ; CC1(C)c2cc3ccccc3cc2-c2c(-c3ccc(-c4cc(-c5cccc(-c6ccccc6)c5)nc(-c5ccccc5)n4)c4ccccc34)cccc21 ; CC1(C)c2cc3ccccc3cc2-c2c(-c3ccc(-c4cc(-c5ccccc5-c5ccccc5)nc(-c5ccccc5)n4)c4ccccc34)cccc21]

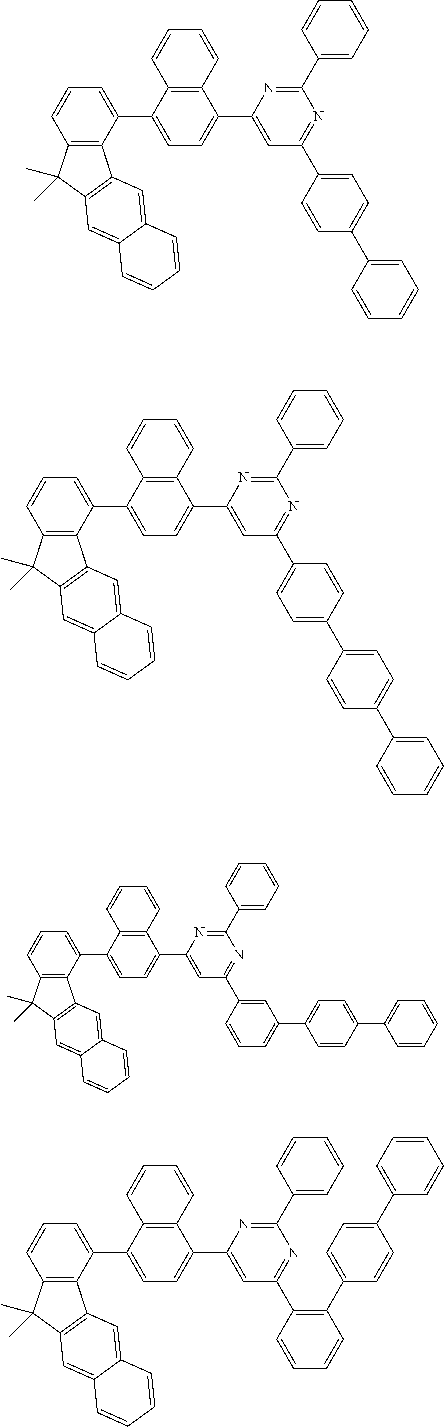 [SMILES: CC1(C)c2cc3ccccc3cc2-c2c(-c3ccc(-c4cc(-c5ccc(-c6ccccc6)cc5)nc(-c5ccccc5)n4)c4ccccc34)cccc21 ; CC1(C)c2cc3ccccc3cc2-c2c(-c3ccc(-c4cc(-c5ccc(-c6ccc(-c7ccccc7)cc6)cc5)nc(-c5ccccc5)n4)c4ccccc34)cccc21 ; CC1(C)c2cc3ccccc3cc2-c2c(-c3ccc(-c4cc(-c5cccc(-c6ccc(-c7ccccc7)cc6)c5)nc(-c5ccccc5)n4)c4ccccc34)cccc21 ; CC1(C)c2cc3ccccc3cc2-c2c(-c3ccc(-c4cc(-c5ccccc5-c5ccc(-c6ccccc6)cc5)nc(-c5ccccc5)n4)c4ccccc34)cccc21]

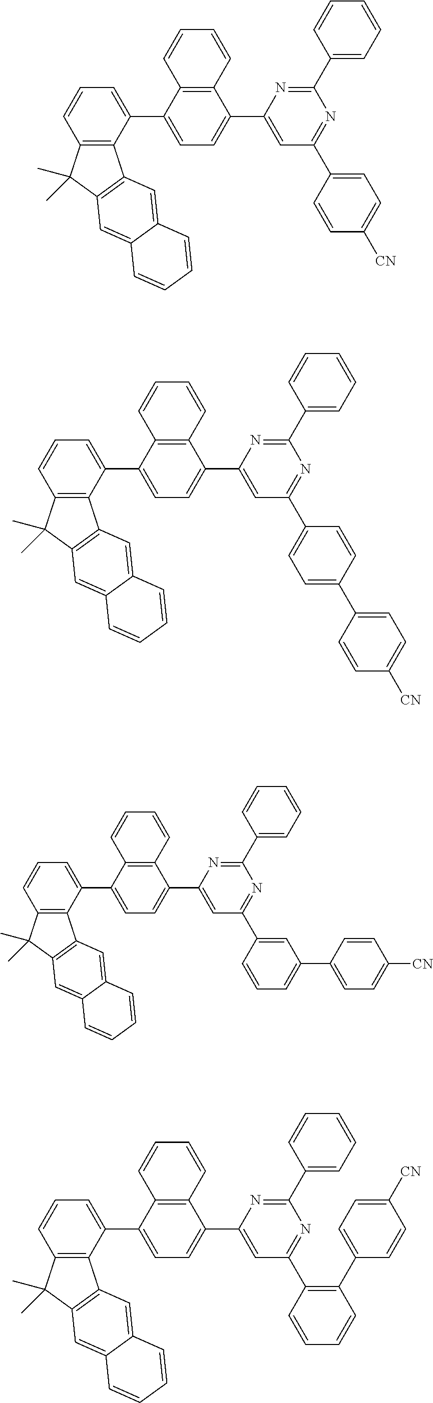 [SMILES: CC1(C)c2cc3ccccc3cc2-c2c(-c3ccc(-c4cc(-c5ccc(C#N)cc5)nc(-c5ccccc5)n4)c4ccccc34)cccc21 ; CC1(C)c2cc3ccccc3cc2-c2c(-c3ccc(-c4cc(-c5ccc(-c6ccc(C#N)cc6)cc5)nc(-c5ccccc5)n4)c4ccccc34)cccc21 ; CC1(C)c2cc3ccccc3cc2-c2c(-c3ccc(-c4cc(-c5cccc(-c6ccc(C#N)cc6)c5)nc(-c5ccccc5)n4)c4ccccc34)cccc21 ; CC1(C)c2cc3ccccc3cc2-c2c(-c3ccc(-c4cc(-c5ccccc5-c5ccc(C#N)cc5)nc(-c5ccccc5)n4)c4ccccc34)cccc21]

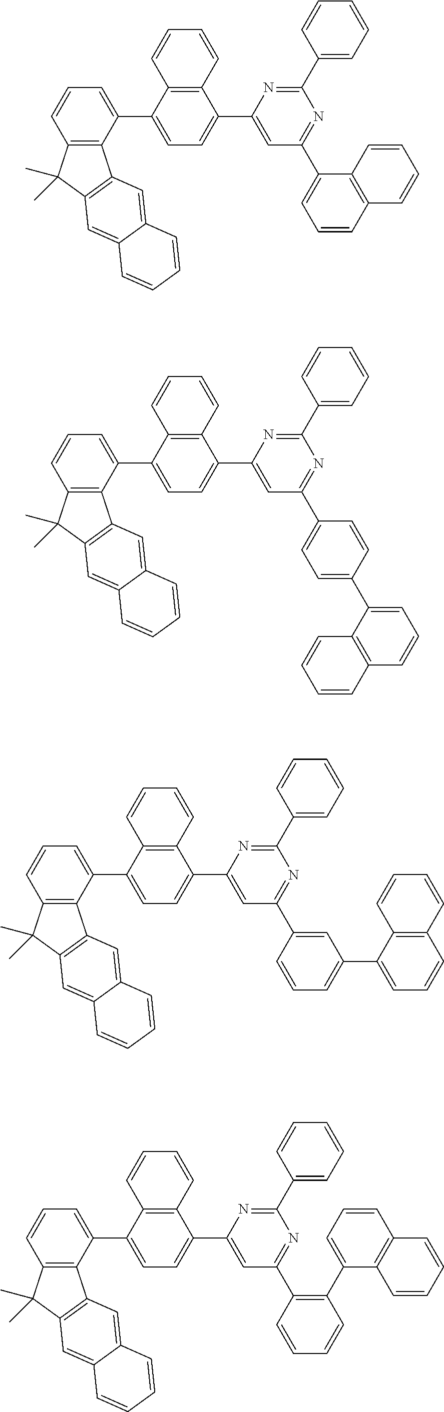 [SMILES: CC1(C)c2cc3ccccc3cc2-c2c(-c3ccc(-c4cc(-c5cccc6ccccc56)nc(-c5ccccc5)n4)c4ccccc34)cccc21 ; CC1(C)c2cc3ccccc3cc2-c2c(-c3ccc(-c4cc(-c5ccc(-c6cccc7ccccc67)cc5)nc(-c5ccccc5)n4)c4ccccc34)cccc21 ; CC1(C)c2cc3ccccc3cc2-c2c(-c3ccc(-c4cc(-c5cccc(-c6cccc7ccccc67)c5)nc(-c5ccccc5)n4)c4ccccc34)cccc21 ; CC1(C)c2cc3ccccc3cc2-c2c(-c3ccc(-c4cc(-c5ccccc5-c5cccc6ccccc56)nc(-c5ccccc5)n4)c4ccccc34)cccc21]

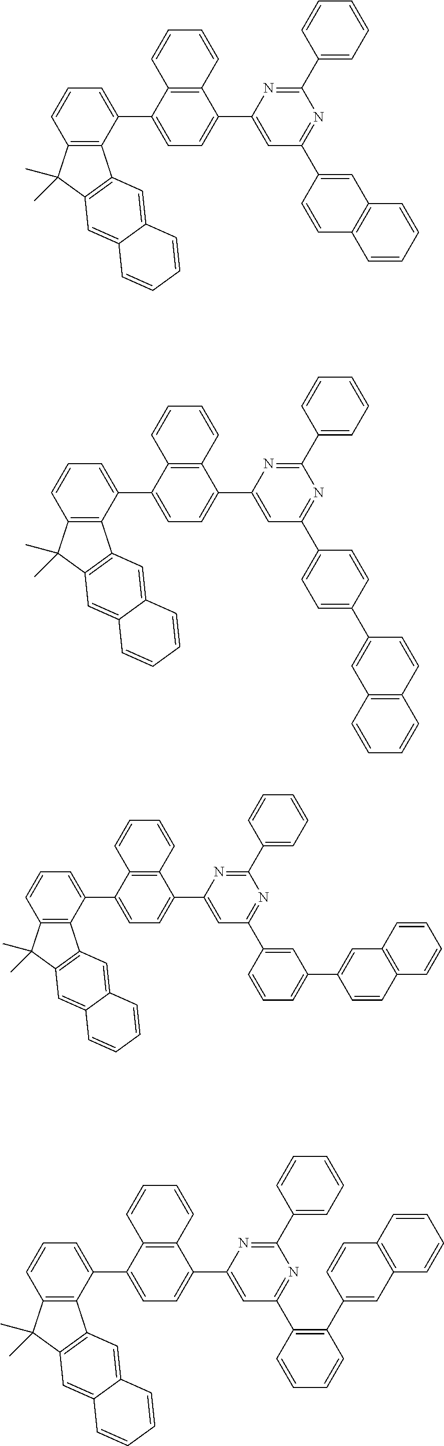 [SMILES: CC1(C)c2cc3ccccc3cc2-c2c(-c3ccc(-c4cc(-c5ccc6ccccc6c5)nc(-c5ccccc5)n4)c4ccccc34)cccc21 ; CC1(C)c2cc3ccccc3cc2-c2c(-c3ccc(-c4cc(-c5ccc(-c6ccc7ccccc7c6)cc5)nc(-c5ccccc5)n4)c4ccccc34)cccc21 ; CC1(C)c2cc3ccccc3cc2-c2c(-c3ccc(-c4cc(-c5cccc(-c6ccc7ccccc7c6)c5)nc(-c5ccccc5)n4)c4ccccc34)cccc21 ; CC1(C)c2cc3ccccc3cc2-c2c(-c3ccc(-c4cc(-c5ccccc5-c5ccc6ccccc6c5)nc(-c5ccccc5)n4)c4ccccc34)cccc21]

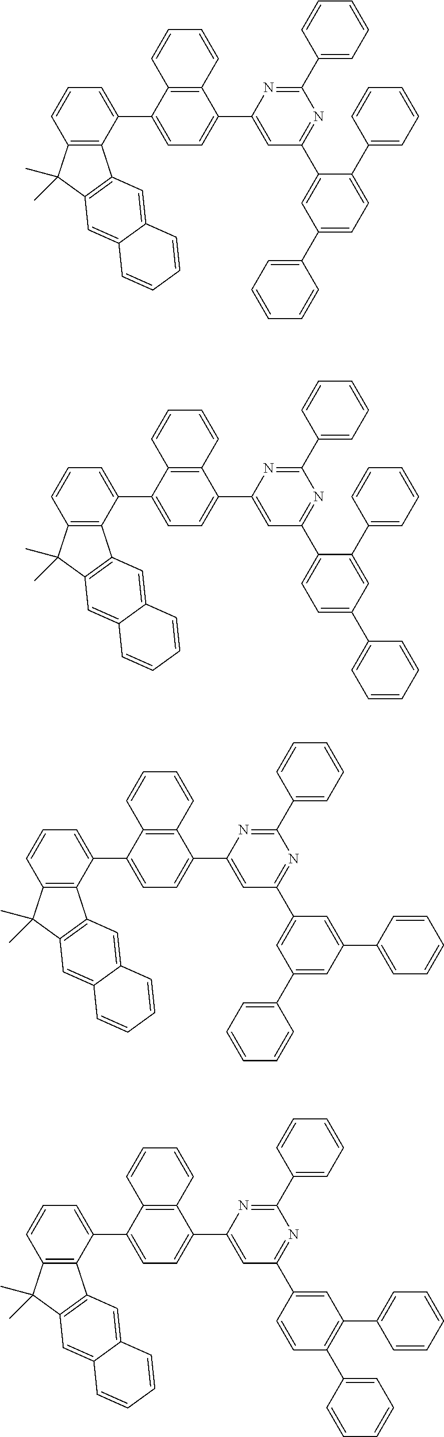 [SMILES: CC1(C)c2cc3ccccc3cc2-c2c(-c3ccc(-c4cc(-c5cc(-c6ccccc6)ccc5-c5ccccc5)nc(-c5ccccc5)n4)c4ccccc34)cccc21 ; CC1(C)c2cc3ccccc3cc2-c2c(-c3ccc(-c4cc(-c5ccc(-c6ccccc6)cc5-c5ccccc5)nc(-c5ccccc5)n4)c4ccccc34)cccc21 ; CC1(C)c2cc3ccccc3cc2-c2c(-c3ccc(-c4cc(-c5cc(-c6ccccc6)cc(-c6ccccc6)c5)nc(-c5ccccc5)n4)c4ccccc34)cccc21 ; CC1(C)c2cc3ccccc3cc2-c2c(-c3ccc(-c4cc(-c5ccc(-c6ccccc6)c(-c6ccccc6)c5)nc(-c5ccccc5)n4)c4ccccc34)cccc21]

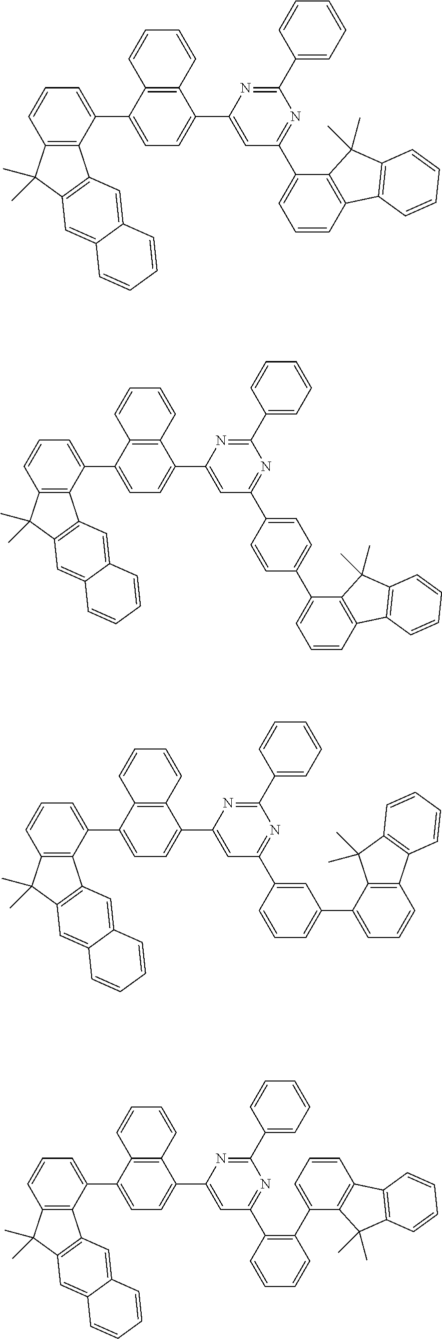 [SMILES: CC1(C)c2cc3ccccc3cc2-c2c(-c3ccc(-c4cc(-c5cccc6c5C(C)(C)c5ccccc5-6)nc(-c5ccccc5)n4)c4ccccc34)cccc21 ; CC1(C)c2cc3ccccc3cc2-c2c(-c3ccc(-c4cc(-c5ccc(-c6cccc7c6C(C)(C)c6ccccc6-7)cc5)nc(-c5ccccc5)n4)c4ccccc34)cccc21 ; CC1(C)c2cc3ccccc3cc2-c2c(-c3ccc(-c4cc(-c5cccc(-c6cccc7c6C(C)(C)c6ccccc6-7)c5)nc(-c5ccccc5)n4)c4ccccc34)cccc21 ; CC1(C)c2cc3ccccc3cc2-c2c(-c3ccc(-c4cc(-c5ccccc5-c5cccc6c5C(C)(C)c5ccccc5-6)nc(-c5ccccc5)n4)c4ccccc34)cccc21]

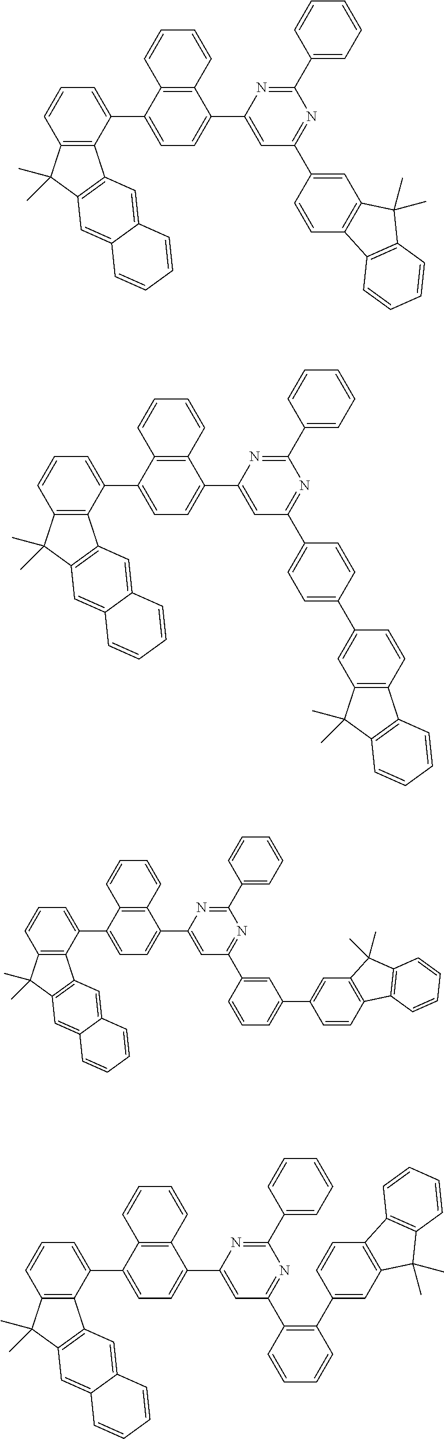

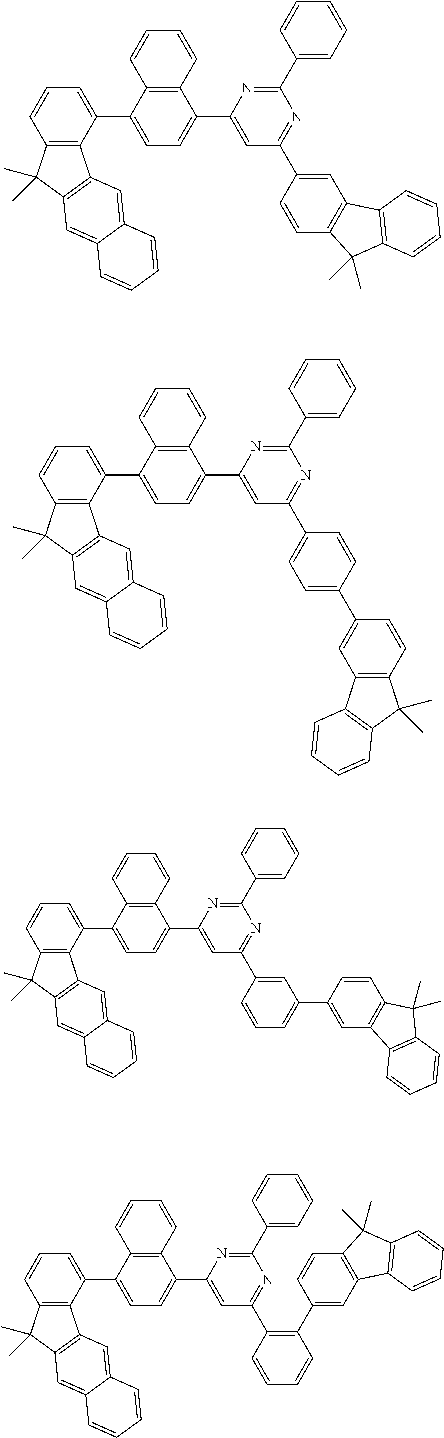

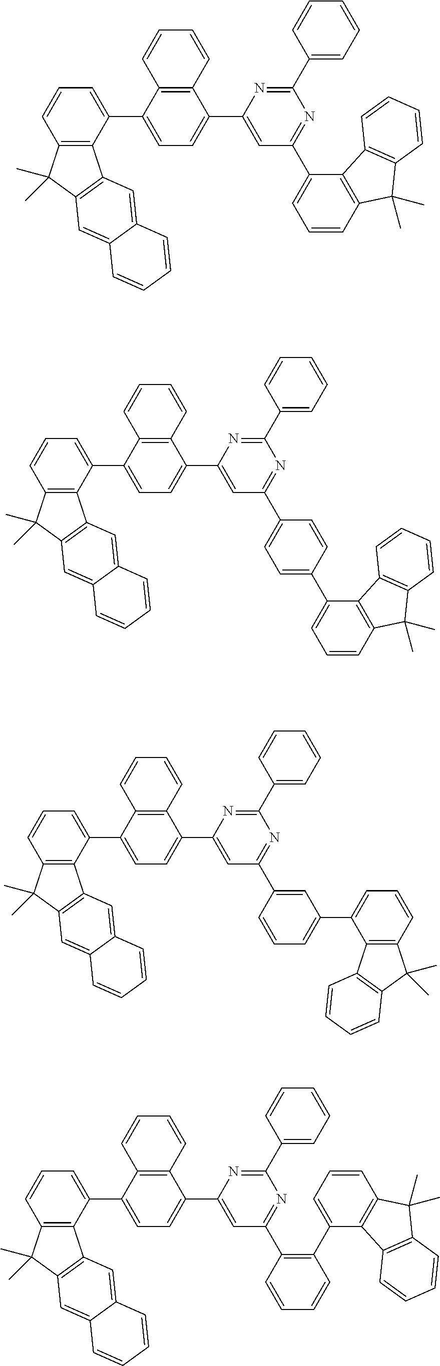

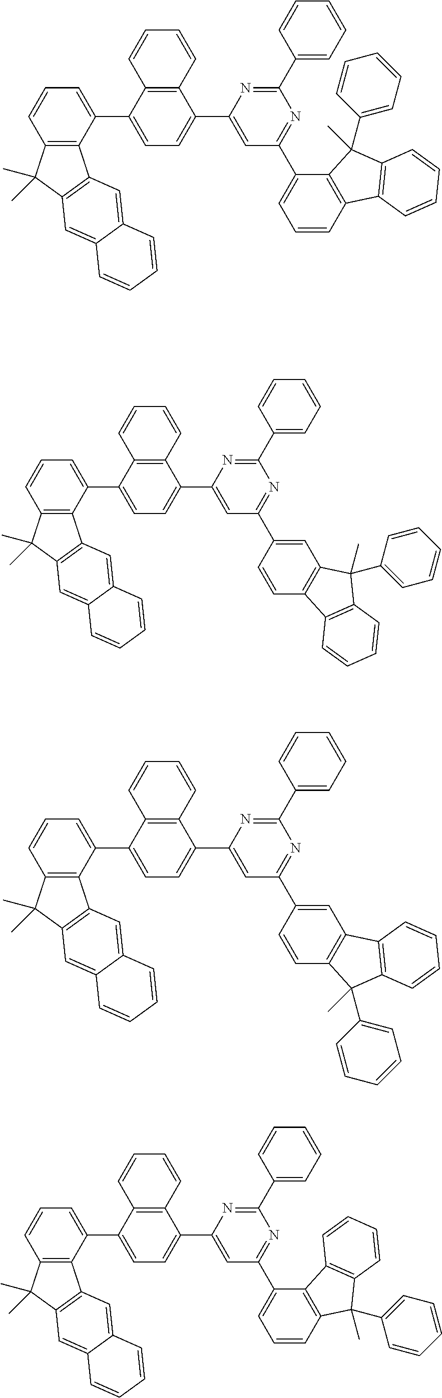 [SMILES: CC1(C)c2cc3ccccc3cc2-c2c(-c3ccc(-c4cc(-c5cccc6c5C(C)(c5ccccc5)c5ccccc5-6)nc(-c5ccccc5)n4)c4ccccc34)cccc21 ; CC1(C)c2cc3ccccc3cc2-c2c(-c3ccc(-c4cc(-c5ccc6c(c5)C(C)(c5ccccc5)c5ccccc5-6)nc(-c5ccccc5)n4)c4ccccc34)cccc21 ; CC1(C)c2cc3ccccc3cc2-c2c(-c3ccc(-c4cc(-c5ccc6c(c5)-c5ccccc5C6(C)c5ccccc5)nc(-c5ccccc5)n4)c4ccccc34)cccc21 ; CC1(C)c2cc3ccccc3cc2-c2c(-c3ccc(-c4cc(-c5cccc6c5-c5ccccc5C6(C)c5ccccc5)nc(-c5ccccc5)n4)c4ccccc34)cccc21]

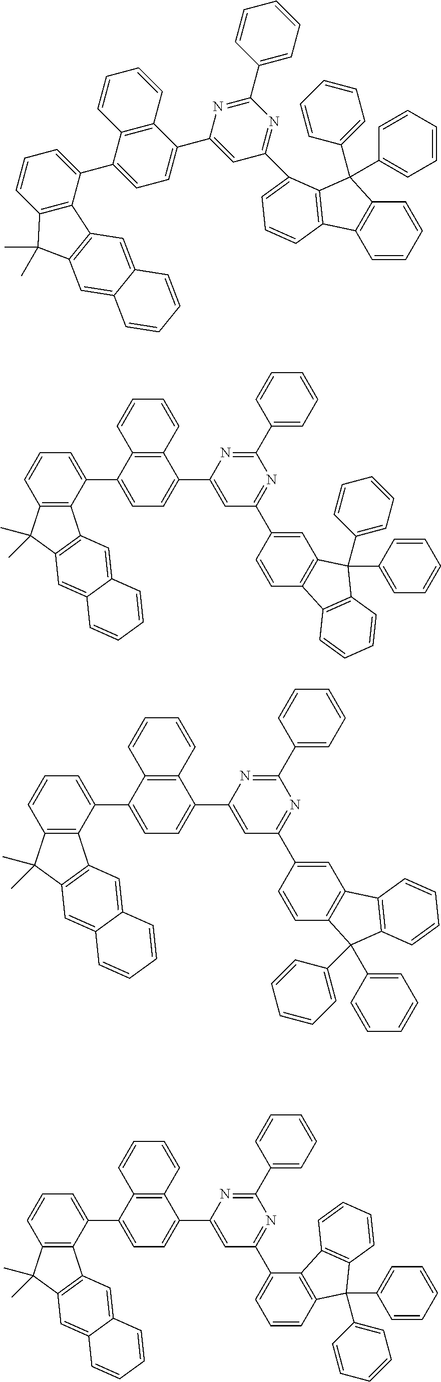

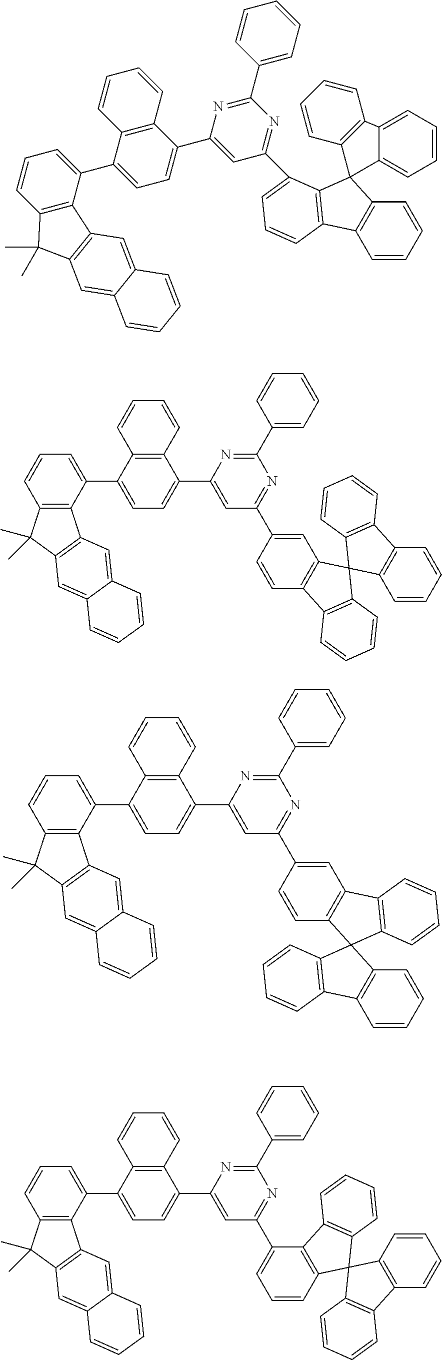

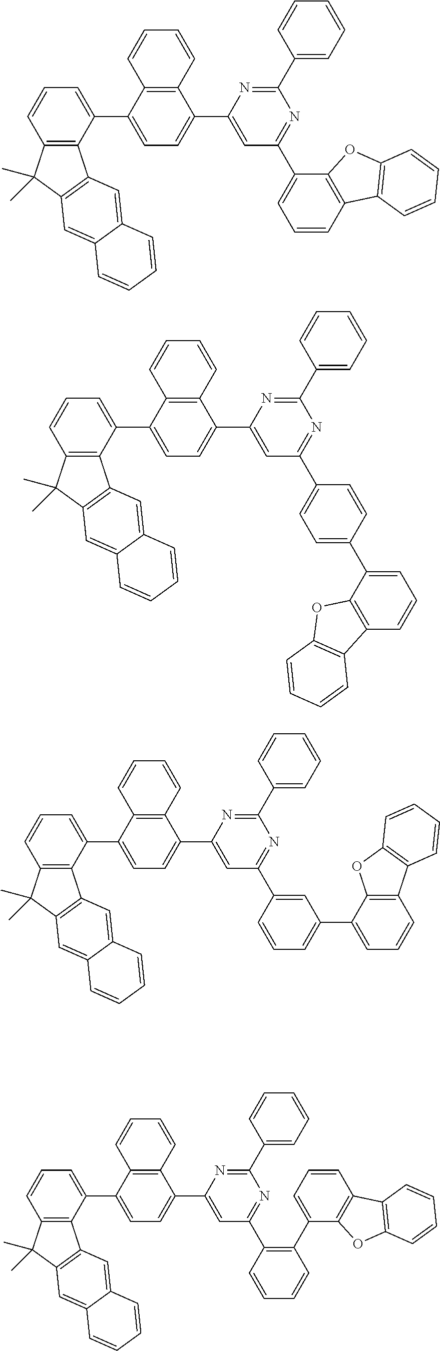

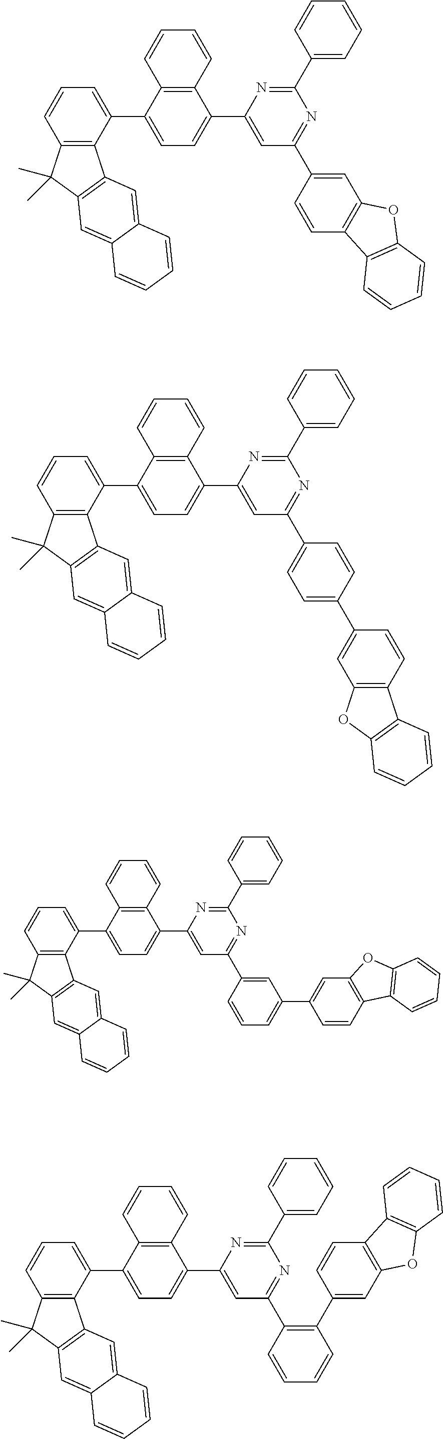 [SMILES: CC1(C)c2cc3ccccc3cc2-c2c(-c3ccc(-c4cc(-c5ccc6c(c5)oc5ccccc56)nc(-c5ccccc5)n4)c4ccccc34)cccc21 ; CC1(C)c2cc3ccccc3cc2-c2c(-c3ccc(-c4cc(-c5ccc(-c6ccc7c(c6)oc6ccccc67)cc5)nc(-c5ccccc5)n4)c4ccccc34)cccc21 ; CC1(C)c2cc3ccccc3cc2-c2c(-c3ccc(-c4cc(-c5cccc(-c6ccc7c(c6)oc6ccccc67)c5)nc(-c5ccccc5)n4)c4ccccc34)cccc21 ; CC1(C)c2cc3ccccc3cc2-c2c(-c3ccc(-c4cc(-c5ccccc5-c5ccc6c(c5)oc5ccccc56)nc(-c5ccccc5)n4)c4ccccc34)cccc21]

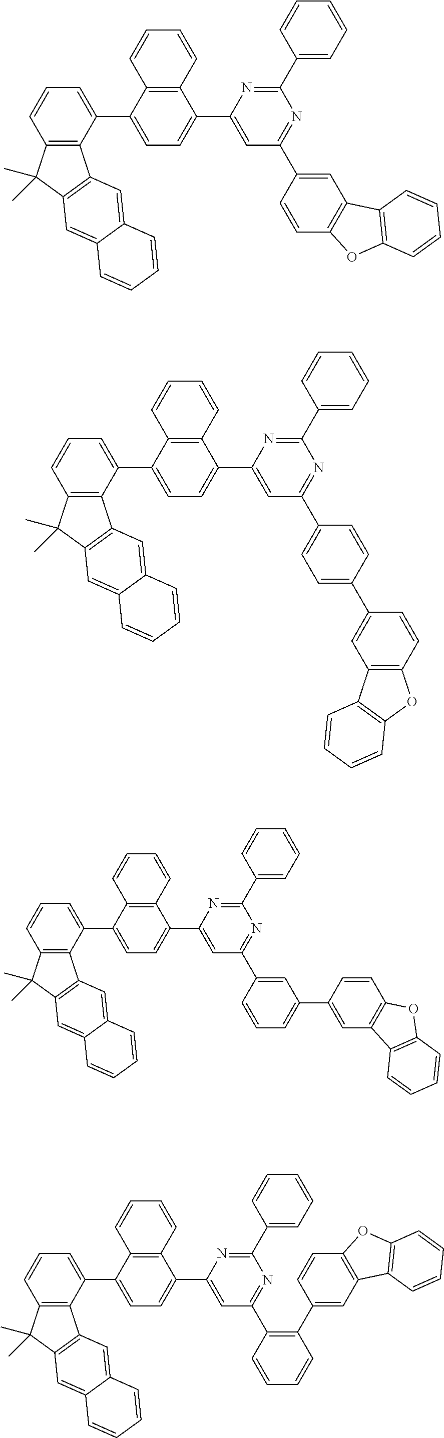 [SMILES: CC1(C)c2cc3ccccc3cc2-c2c(-c3ccc(-c4cc(-c5ccc6oc7ccccc7c6c5)nc(-c5ccccc5)n4)c4ccccc34)cccc21 ; CC1(C)c2cc3ccccc3cc2-c2c(-c3ccc(-c4cc(-c5ccc(-c6ccc7oc8ccccc8c7c6)cc5)nc(-c5ccccc5)n4)c4ccccc34)cccc21 ; CC1(C)c2cc3ccccc3cc2-c2c(-c3ccc(-c4cc(-c5cccc(-c6ccc7oc8ccccc8c7c6)c5)nc(-c5ccccc5)n4)c4ccccc34)cccc21 ; CC1(C)c2cc3ccccc3cc2-c2c(-c3ccc(-c4cc(-c5ccccc5-c5ccc6oc7ccccc7c6c5)nc(-c5ccccc5)n4)c4ccccc34)cccc21]

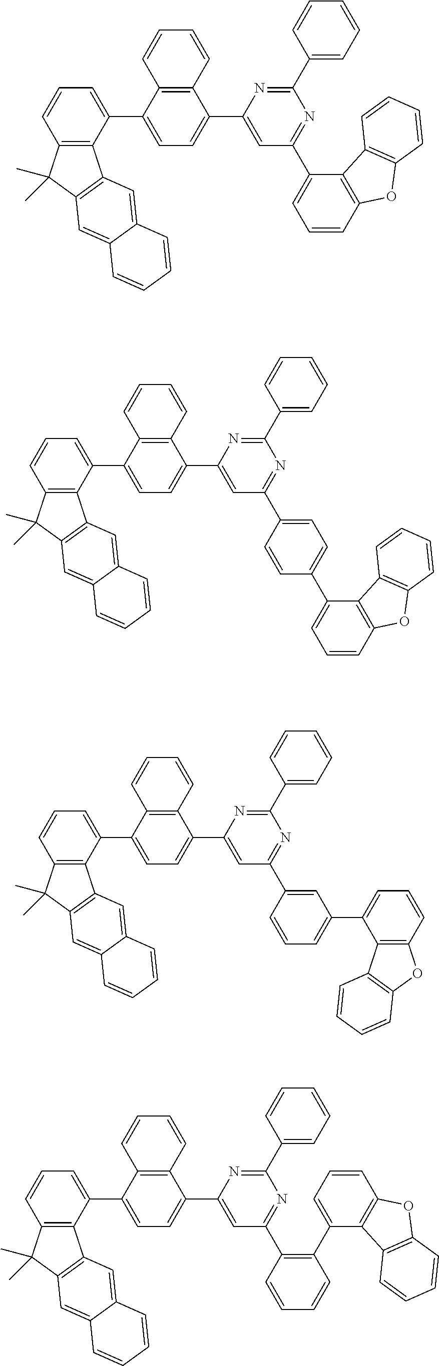 [SMILES: CC1(C)c2cc3ccccc3cc2-c2c(-c3ccc(-c4cc(-c5cccc6oc7ccccc7c56)nc(-c5ccccc5)n4)c4ccccc34)cccc21 ; CC1(C)c2cc3ccccc3cc2-c2c(-c3ccc(-c4cc(-c5ccc(-c6cccc7oc8ccccc8c67)cc5)nc(-c5ccccc5)n4)c4ccccc34)cccc21 ; CC1(C)c2cc3ccccc3cc2-c2c(-c3ccc(-c4cc(-c5cccc(-c6cccc7oc8ccccc8c67)c5)nc(-c5ccccc5)n4)c4ccccc34)cccc21 ; CC1(C)c2cc3ccccc3cc2-c2c(-c3ccc(-c4cc(-c5ccccc5-c5cccc6oc7ccccc7c56)nc(-c5ccccc5)n4)c4ccccc34)cccc21]

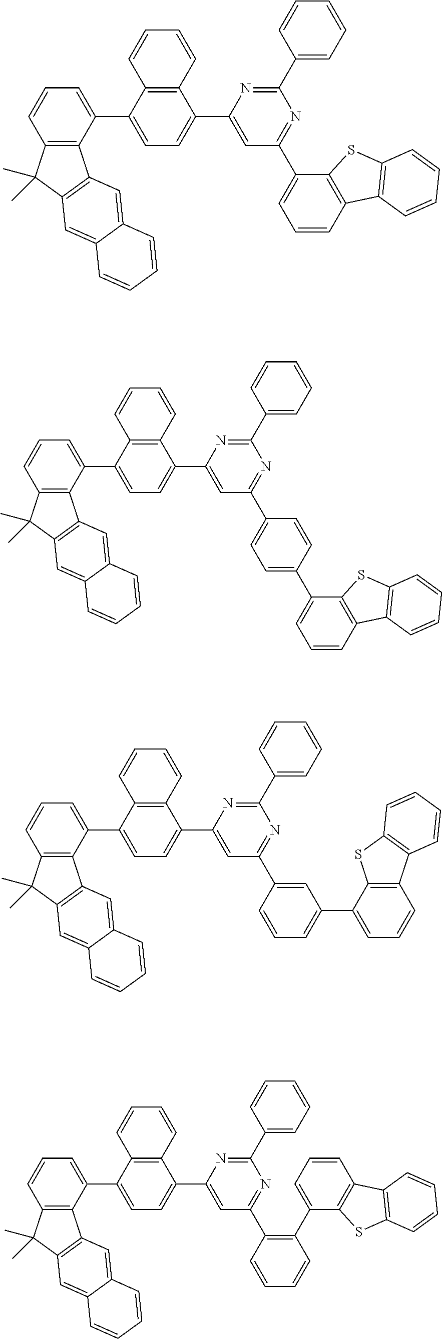 [SMILES: CC1(C)c2cc3ccccc3cc2-c2c(-c3ccc(-c4cc(-c5cccc6c5sc5ccccc56)nc(-c5ccccc5)n4)c4ccccc34)cccc21 ; CC1(C)c2cc3ccccc3cc2-c2c(-c3ccc(-c4cc(-c5ccc(-c6cccc7c6sc6ccccc67)cc5)nc(-c5ccccc5)n4)c4ccccc34)cccc21 ; CC1(C)c2cc3ccccc3cc2-c2c(-c3ccc(-c4cc(-c5cccc(-c6cccc7c6sc6ccccc67)c5)nc(-c5ccccc5)n4)c4ccccc34)cccc21 ; CC1(C)c2cc3ccccc3cc2-c2c(-c3ccc(-c4cc(-c5ccccc5-c5cccc6c5sc5ccccc56)nc(-c5ccccc5)n4)c4ccccc34)cccc21]

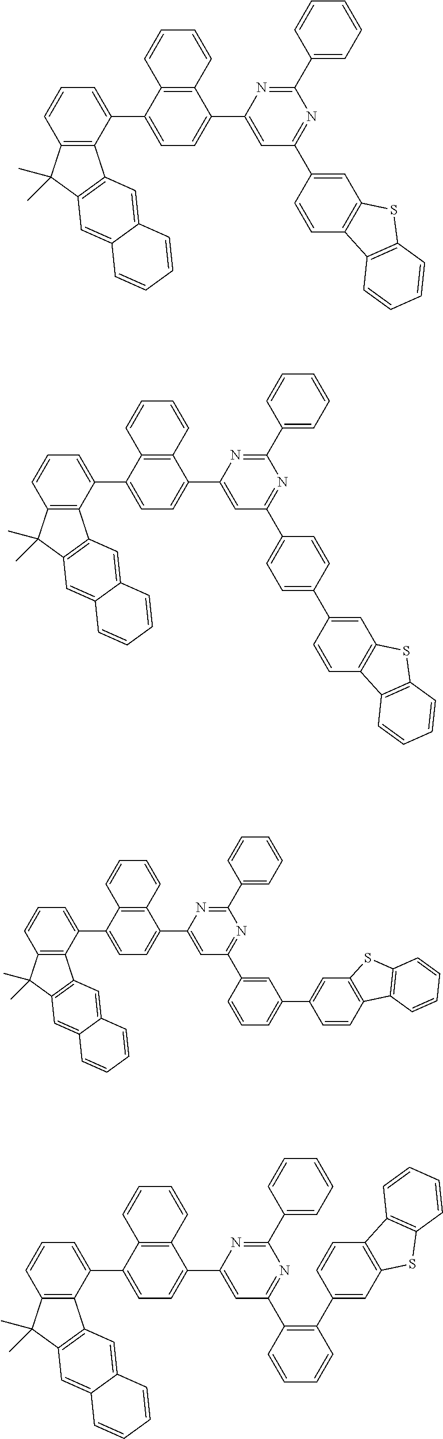 [SMILES: CC1(C)c2cc3ccccc3cc2-c2c(-c3ccc(-c4cc(-c5ccc6c(c5)sc5ccccc56)nc(-c5ccccc5)n4)c4ccccc34)cccc21 ; CC1(C)c2cc3ccccc3cc2-c2c(-c3ccc(-c4cc(-c5ccc(-c6ccc7c(c6)sc6ccccc67)cc5)nc(-c5ccccc5)n4)c4ccccc34)cccc21 ; CC1(C)c2cc3ccccc3cc2-c2c(-c3ccc(-c4cc(-c5cccc(-c6ccc7c(c6)sc6ccccc67)c5)nc(-c5ccccc5)n4)c4ccccc34)cccc21 ; CC1(C)c2cc3ccccc3cc2-c2c(-c3ccc(-c4cc(-c5ccccc5-c5ccc6c(c5)sc5ccccc56)nc(-c5ccccc5)n4)c4ccccc34)cccc21]

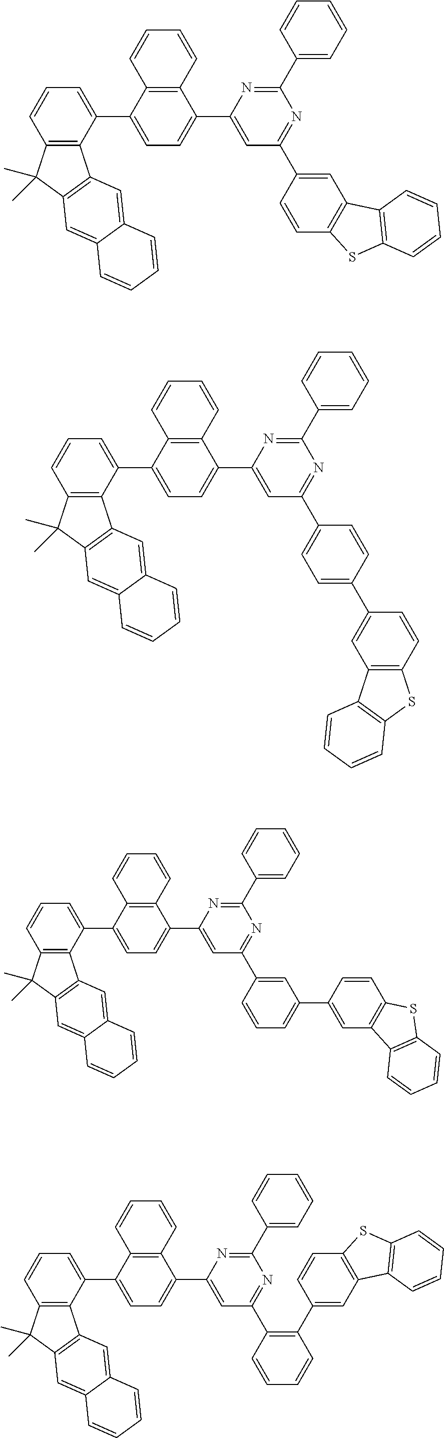 [SMILES: CC1(C)c2cc3ccccc3cc2-c2c(-c3ccc(-c4cc(-c5ccc6sc7ccccc7c6c5)nc(-c5ccccc5)n4)c4ccccc34)cccc21 ; CC1(C)c2cc3ccccc3cc2-c2c(-c3ccc(-c4cc(-c5ccc(-c6ccc7sc8ccccc8c7c6)cc5)nc(-c5ccccc5)n4)c4ccccc34)cccc21 ; CC1(C)c2cc3ccccc3cc2-c2c(-c3ccc(-c4cc(-c5cccc(-c6ccc7sc8ccccc8c7c6)c5)nc(-c5ccccc5)n4)c4ccccc34)cccc21 ; CC1(C)c2cc3ccccc3cc2-c2c(-c3ccc(-c4cc(-c5ccccc5-c5ccc6sc7ccccc7c6c5)nc(-c5ccccc5)n4)c4ccccc34)cccc21]

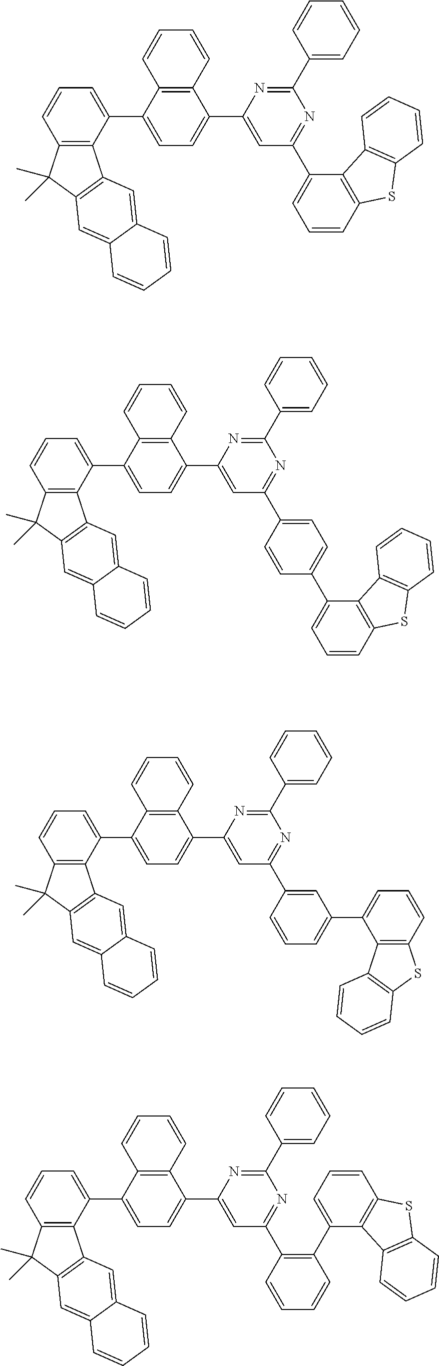 [SMILES: CC1(C)c2cc3ccccc3cc2-c2c(-c3ccc(-c4cc(-c5cccc6sc7ccccc7c56)nc(-c5ccccc5)n4)c4ccccc34)cccc21 ; CC1(C)c2cc3ccccc3cc2-c2c(-c3ccc(-c4cc(-c5ccc(-c6cccc7sc8ccccc8c67)cc5)nc(-c5ccccc5)n4)c4ccccc34)cccc21 ; CC1(C)c2cc3ccccc3cc2-c2c(-c3ccc(-c4cc(-c5cccc(-c6cccc7sc8ccccc8c67)c5)nc(-c5ccccc5)n4)c4ccccc34)cccc21 ; CC1(C)c2cc3ccccc3cc2-c2c(-c3ccc(-c4cc(-c5ccccc5-c5cccc6sc7ccccc7c56)nc(-c5ccccc5)n4)c4ccccc34)cccc21]

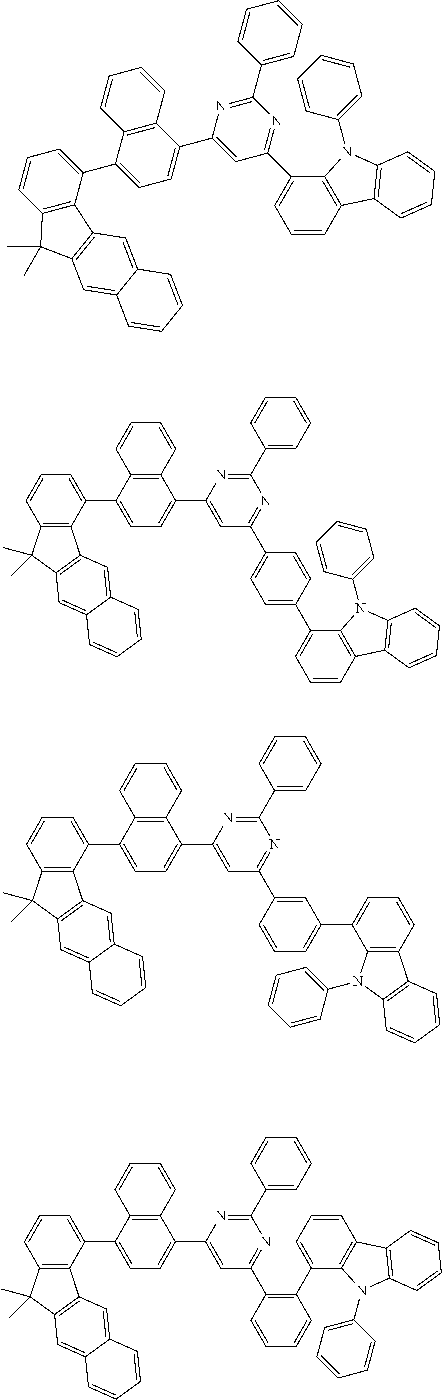 [SMILES: CC1(C)c2cc3ccccc3cc2-c2c(-c3ccc(-c4cc(-c5cccc6c7ccccc7n(-c7ccccc7)c56)nc(-c5ccccc5)n4)c4ccccc34)cccc21 ; CC1(C)c2cc3ccccc3cc2-c2c(-c3ccc(-c4cc(-c5ccc(-c6cccc7c8ccccc8n(-c8ccccc8)c67)cc5)nc(-c5ccccc5)n4)c4ccccc34)cccc21 ; CC1(C)c2cc3ccccc3cc2-c2c(-c3ccc(-c4cc(-c5cccc(-c6cccc7c8ccccc8n(-c8ccccc8)c67)c5)nc(-c5ccccc5)n4)c4ccccc34)cccc21 ; CC1(C)c2cc3ccccc3cc2-c2c(-c3ccc(-c4cc(-c5ccccc5-c5cccc6c7ccccc7n(-c7ccccc7)c56)nc(-c5ccccc5)n4)c4ccccc34)cccc21]

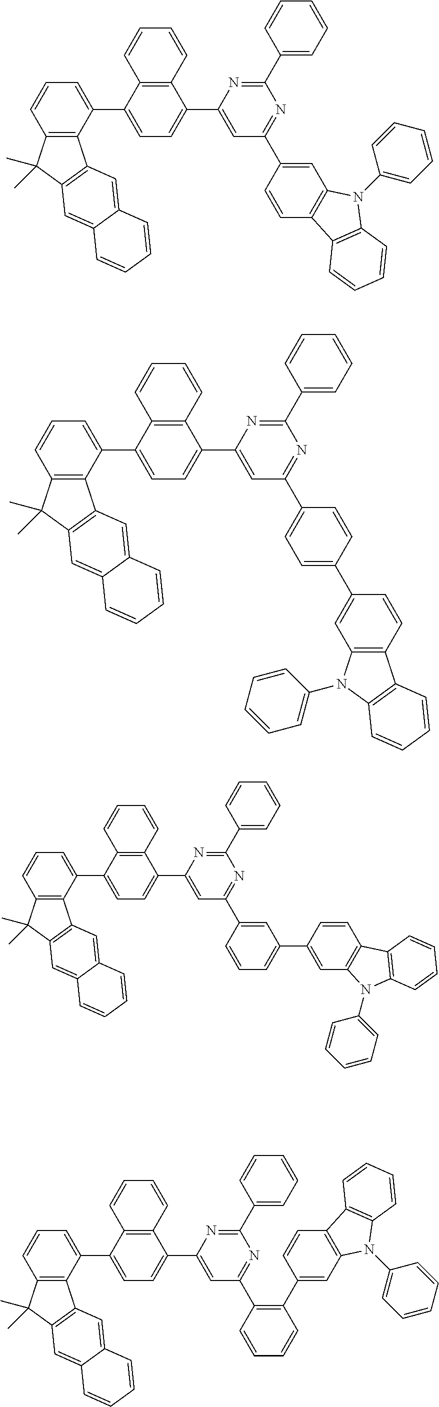 [SMILES: CC1(C)c2cc3ccccc3cc2-c2c(-c3ccc(-c4cc(-c5ccc6c7ccccc7n(-c7ccccc7)c6c5)nc(-c5ccccc5)n4)c4ccccc34)cccc21 ; CC1(C)c2cc3ccccc3cc2-c2c(-c3ccc(-c4cc(-c5ccc(-c6ccc7c8ccccc8n(-c8ccccc8)c7c6)cc5)nc(-c5ccccc5)n4)c4ccccc34)cccc21 ; CC1(C)c2cc3ccccc3cc2-c2c(-c3ccc(-c4cc(-c5cccc(-c6ccc7c8ccccc8n(-c8ccccc8)c7c6)c5)nc(-c5ccccc5)n4)c4ccccc34)cccc21 ; CC1(C)c2cc3ccccc3cc2-c2c(-c3ccc(-c4cc(-c5ccccc5-c5ccc6c7ccccc7n(-c7ccccc7)c6c5)nc(-c5ccccc5)n4)c4ccccc34)cccc21]

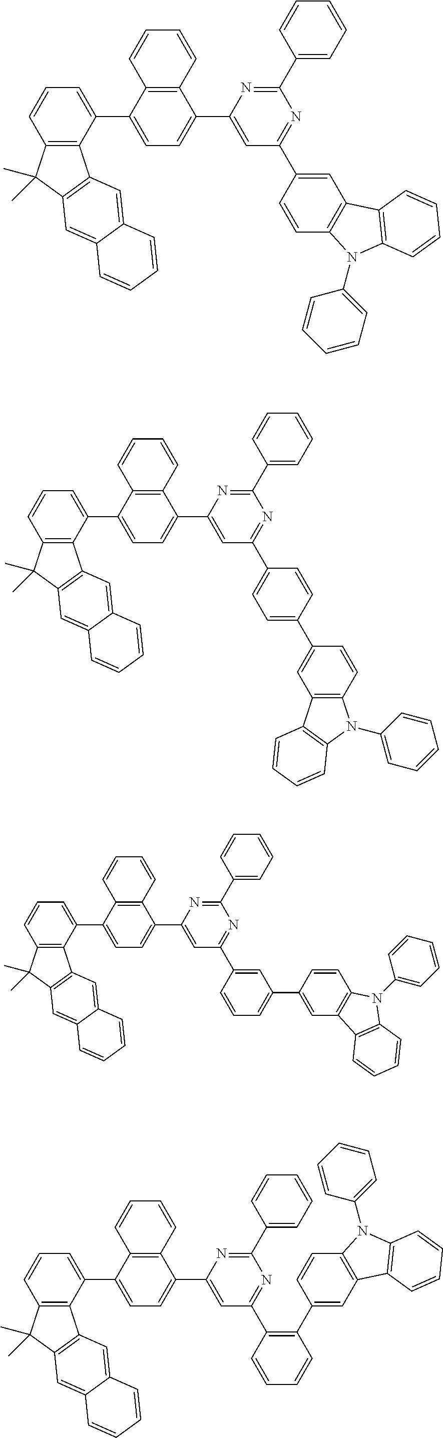 [SMILES: CC1(C)c2cc3ccccc3cc2-c2c(-c3ccc(-c4cc(-c5ccc6c(c5)c5ccccc5n6-c5ccccc5)nc(-c5ccccc5)n4)c4ccccc34)cccc21 ; CC1(C)c2cc3ccccc3cc2-c2c(-c3ccc(-c4cc(-c5ccc(-c6ccc7c(c6)c6ccccc6n7-c6ccccc6)cc5)nc(-c5ccccc5)n4)c4ccccc34)cccc21 ; CC1(C)c2cc3ccccc3cc2-c2c(-c3ccc(-c4cc(-c5cccc(-c6ccc7c(c6)c6ccccc6n7-c6ccccc6)c5)nc(-c5ccccc5)n4)c4ccccc34)cccc21 ; CC1(C)c2cc3ccccc3cc2-c2c(-c3ccc(-c4cc(-c5ccccc5-c5ccc6c(c5)c5ccccc5n6-c5ccccc5)nc(-c5ccccc5)n4)c4ccccc34)cccc21]

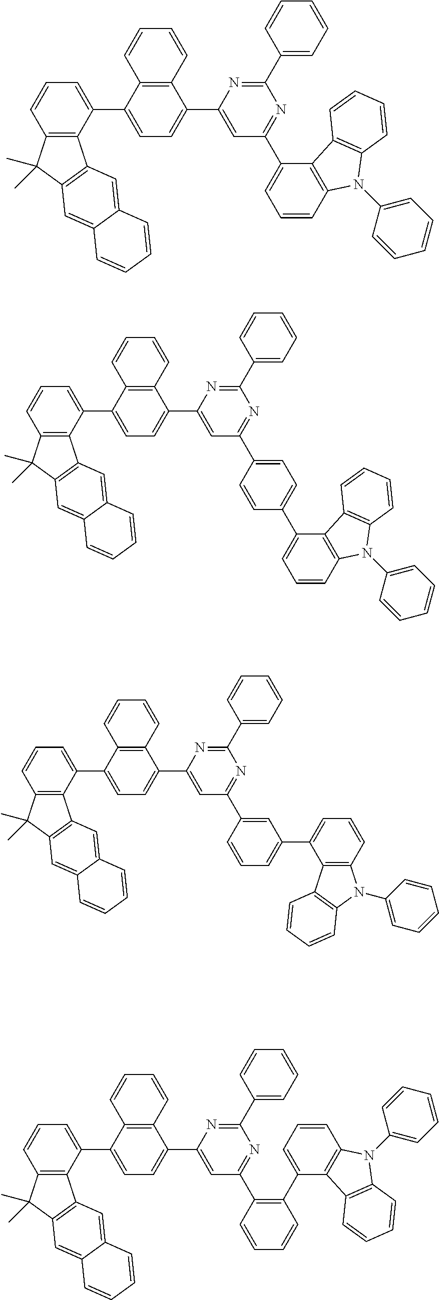 [SMILES: CC1(C)c2cc3ccccc3cc2-c2c(-c3ccc(-c4cc(-c5cccc6c5c5ccccc5n6-c5ccccc5)nc(-c5ccccc5)n4)c4ccccc34)cccc21 ; CC1(C)c2cc3ccccc3cc2-c2c(-c3ccc(-c4cc(-c5ccc(-c6cccc7c6c6ccccc6n7-c6ccccc6)cc5)nc(-c5ccccc5)n4)c4ccccc34)cccc21 ; CC1(C)c2cc3ccccc3cc2-c2c(-c3ccc(-c4cc(-c5cccc(-c6cccc7c6c6ccccc6n7-c6ccccc6)c5)nc(-c5ccccc5)n4)c4ccccc34)cccc21 ; CC1(C)c2cc3ccccc3cc2-c2c(-c3ccc(-c4cc(-c5ccccc5-c5cccc6c5c5ccccc5n6-c5ccccc5)nc(-c5ccccc5)n4)c4ccccc34)cccc21]

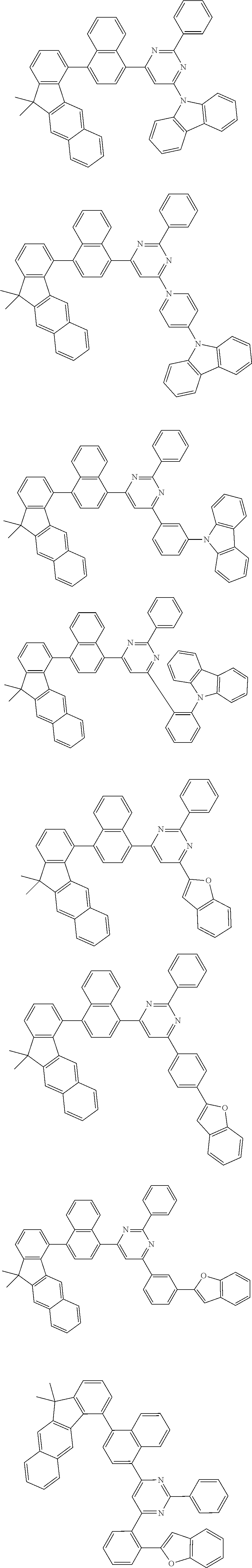

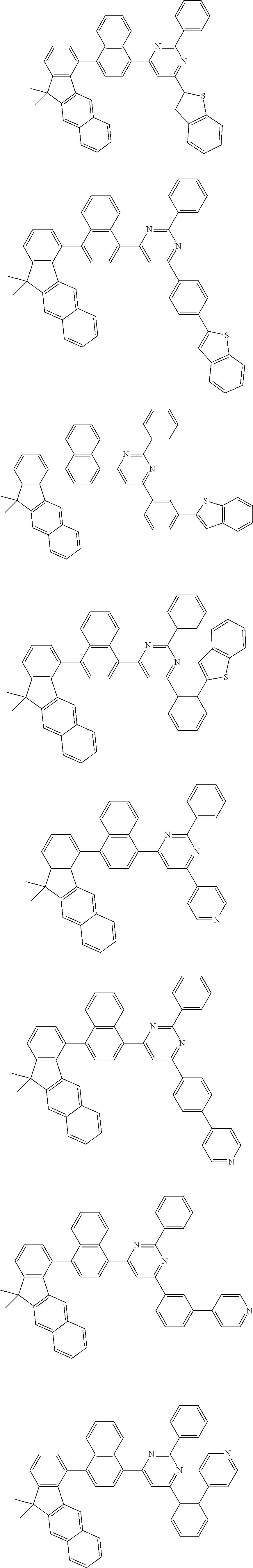

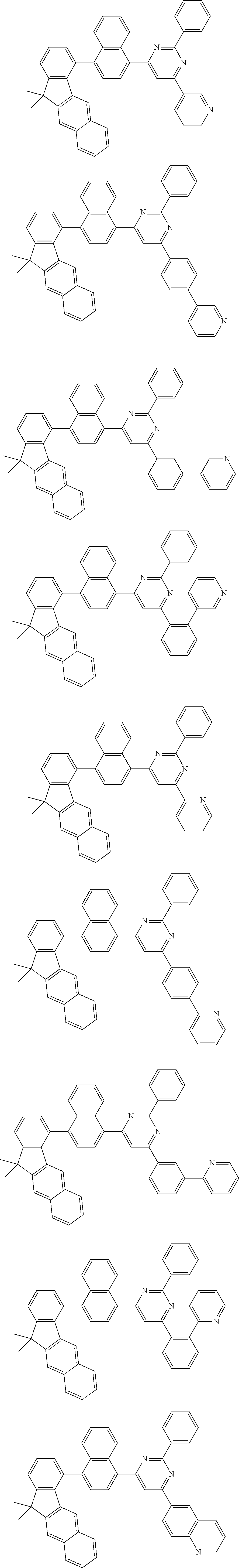

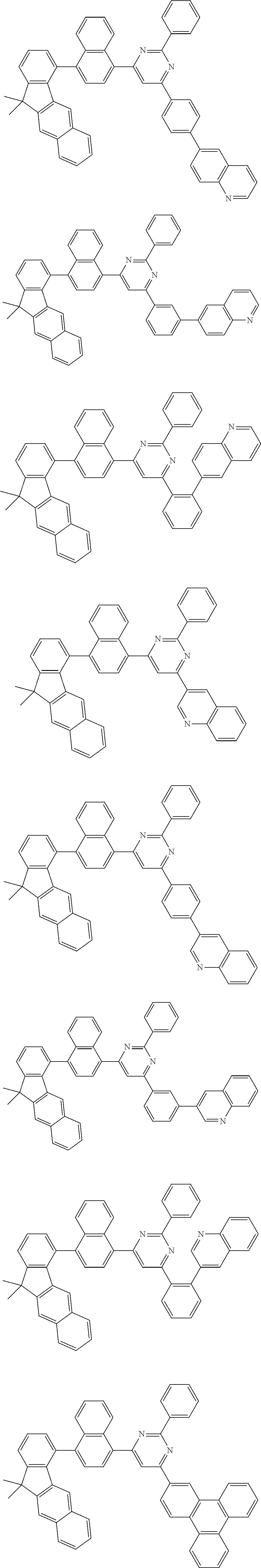

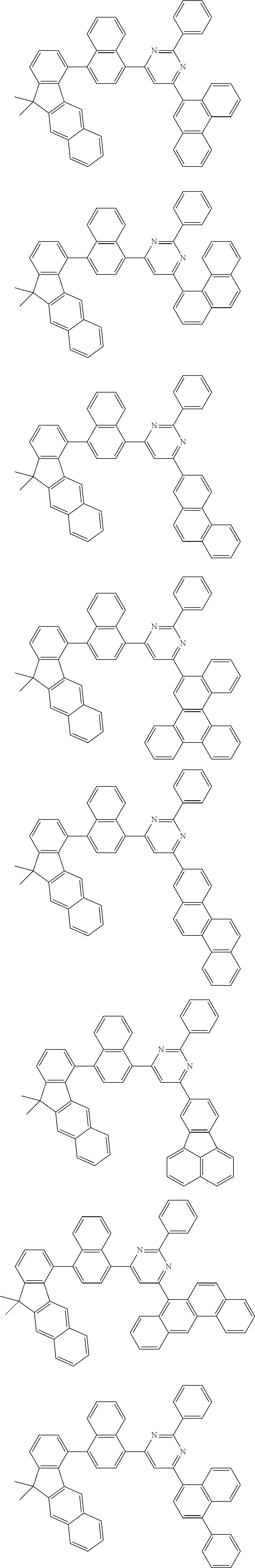

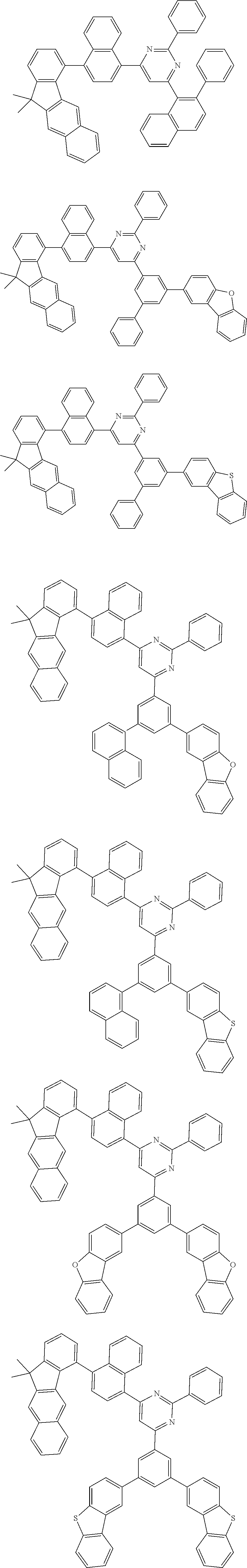

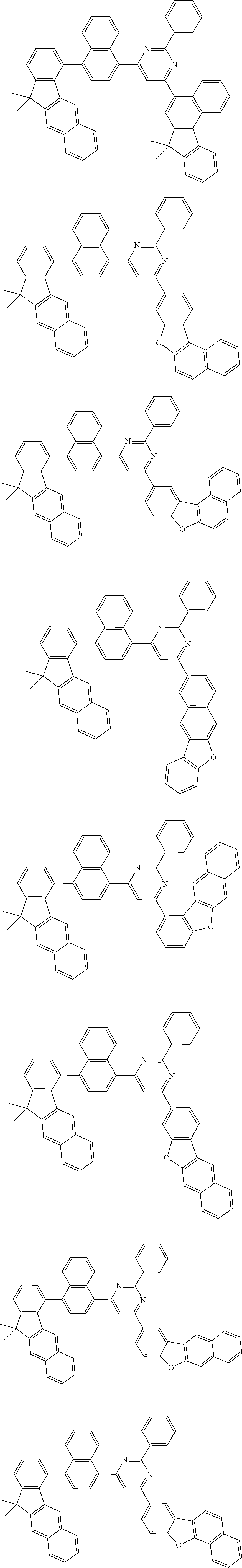

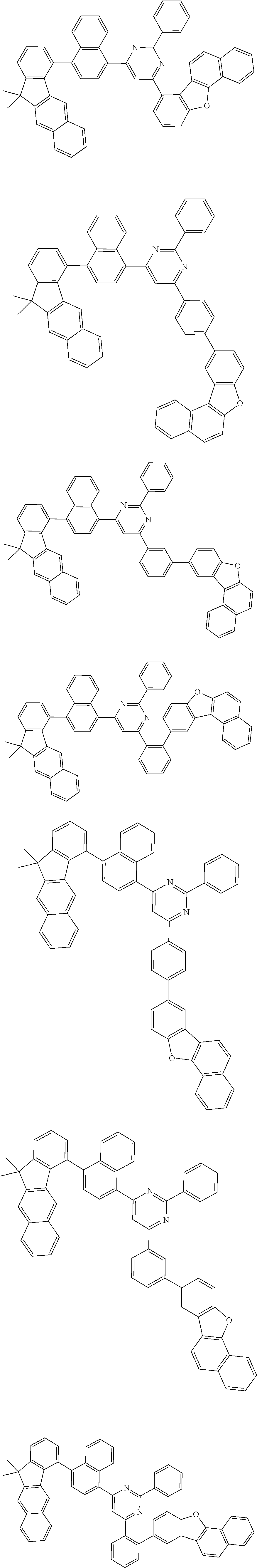

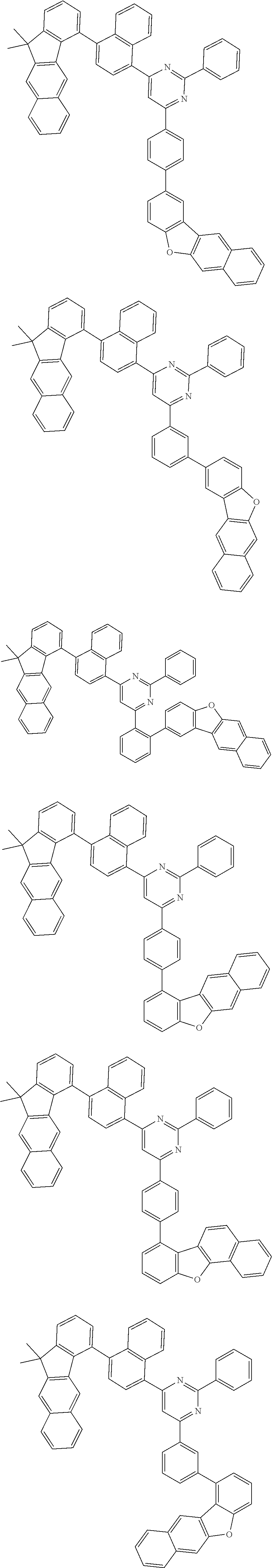

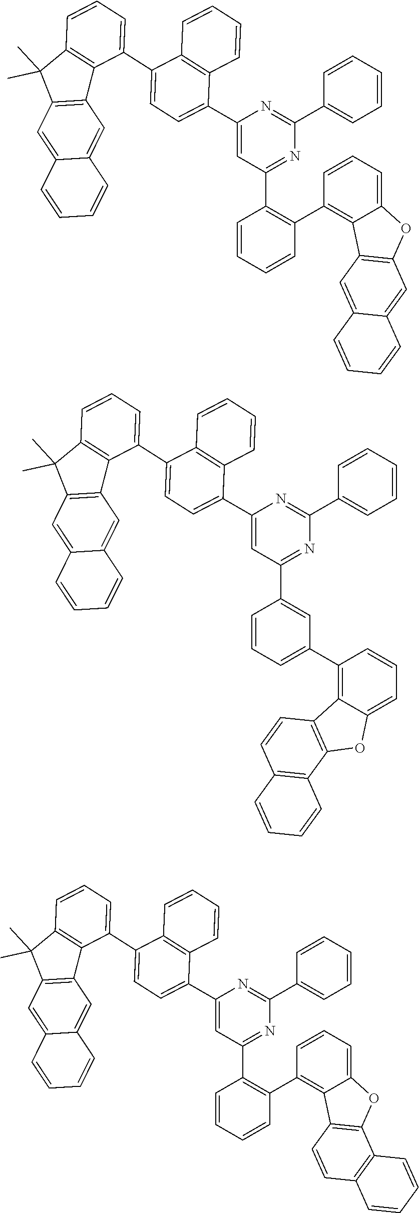 [SMILES: CC1(C)c2cc3ccccc3cc2-c2c(-c3ccc(-c4cc(-c5ccccc5-c5cccc6oc7cc8ccccc8cc7c56)nc(-c5ccccc5)n4)c4ccccc34)cccc21 ; CC1(C)c2cc3ccccc3cc2-c2c(-c3ccc(-c4cc(-c5cccc(-c6cccc7oc8c9ccccc9ccc8c67)c5)nc(-c5ccccc5)n4)c4ccccc34)cccc21 ; CC1(C)c2cc3ccccc3cc2-c2c(-c3ccc(-c4cc(-c5ccccc5-c5cccc6oc7c8ccccc8ccc7c56)nc(-c5ccccc5)n4)c4ccccc34)cccc21]

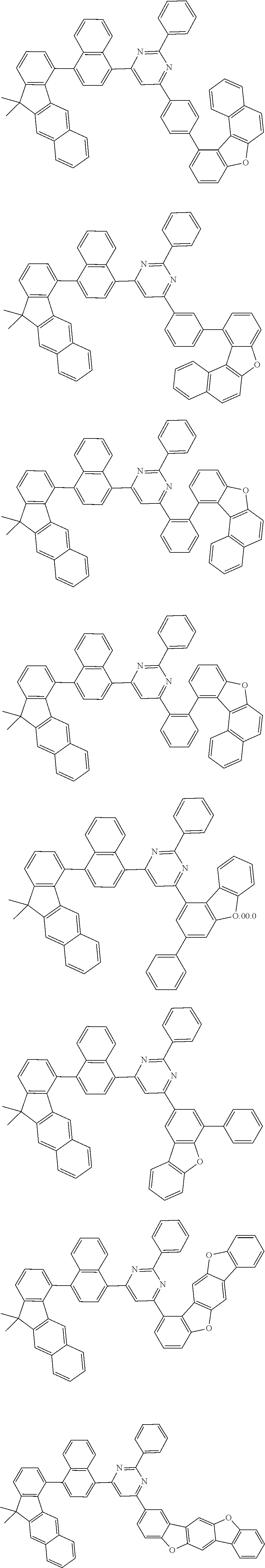

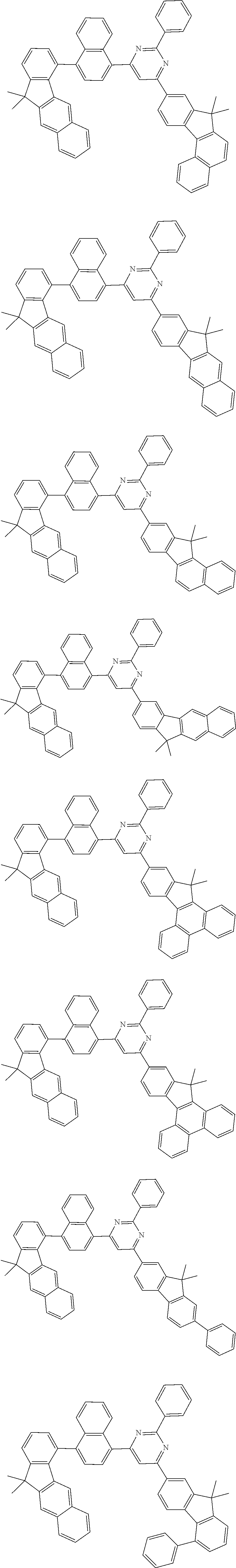

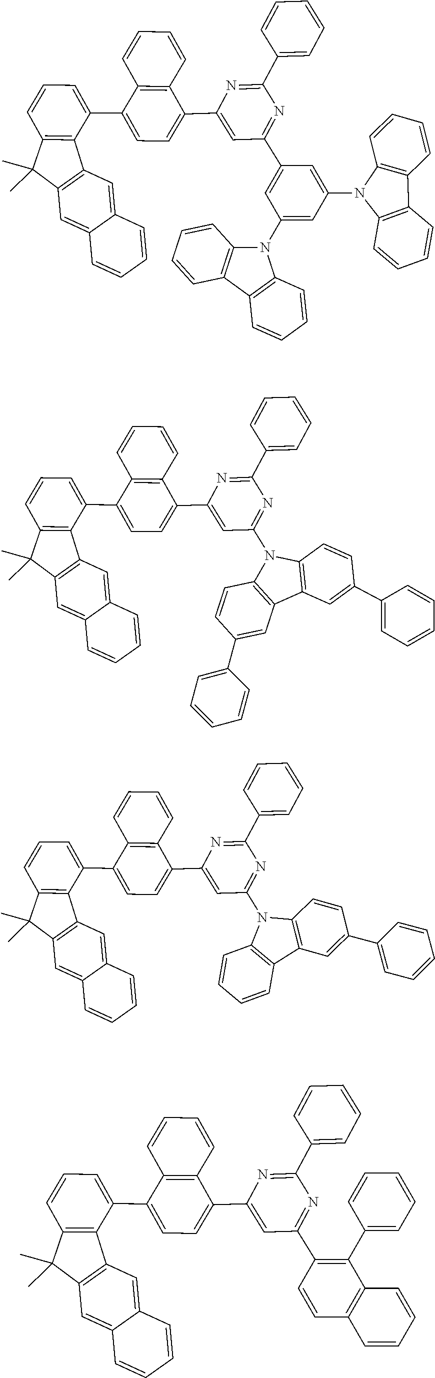 [SMILES: CC1(C)c2cc3ccccc3cc2-c2c(-c3ccc(-c4cc(-c5cc(-n6c7ccccc7c7ccccc76)cc(-n6c7ccccc7c7ccccc76)c5)nc(-c5ccccc5)n4)c4ccccc34)cccc21 ; CC1(C)c2cc3ccccc3cc2-c2c(-c3ccc(-c4cc(-n5c6ccc(-c7ccccc7)cc6c6cc(-c7ccccc7)ccc65)nc(-c5ccccc5)n4)c4ccccc34)cccc21 ; CC1(C)c2cc3ccccc3cc2-c2c(-c3ccc(-c4cc(-n5c6ccccc6c6cc(-c7ccccc7)ccc65)nc(-c5ccccc5)n4)c4ccccc34)cccc21 ; CC1(C)c2cc3ccccc3cc2-c2c(-c3ccc(-c4cc(-c5ccc6ccccc6c5-c5ccccc5)nc(-c5ccccc5)n4)c4ccccc34)cccc21]

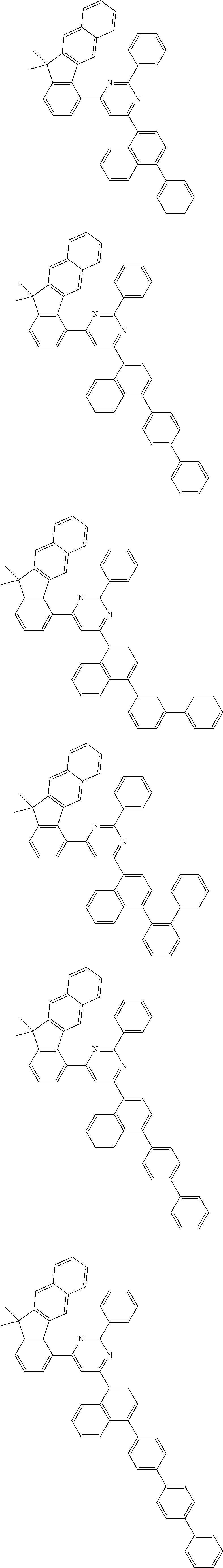

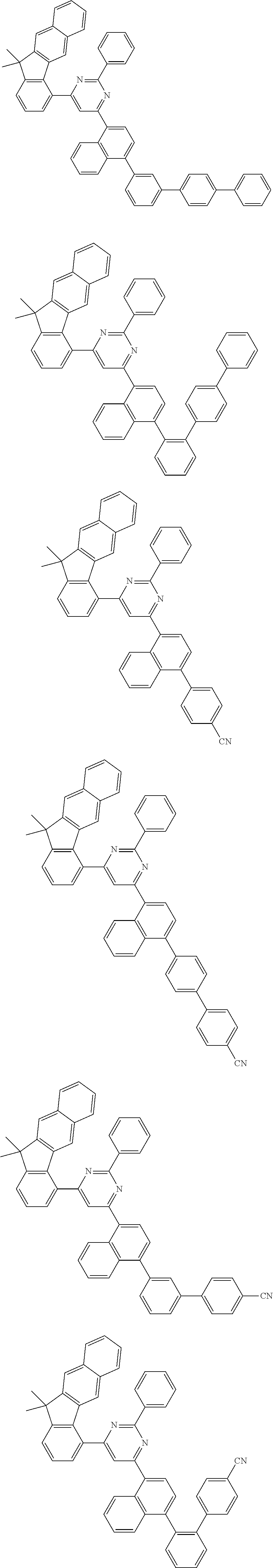

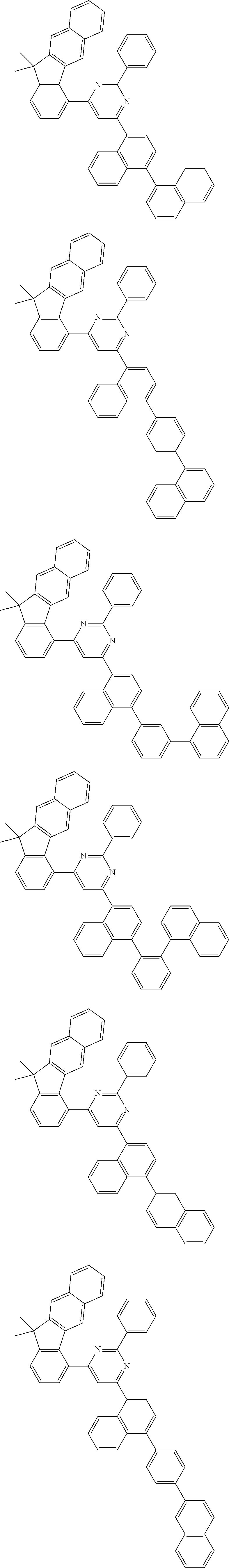

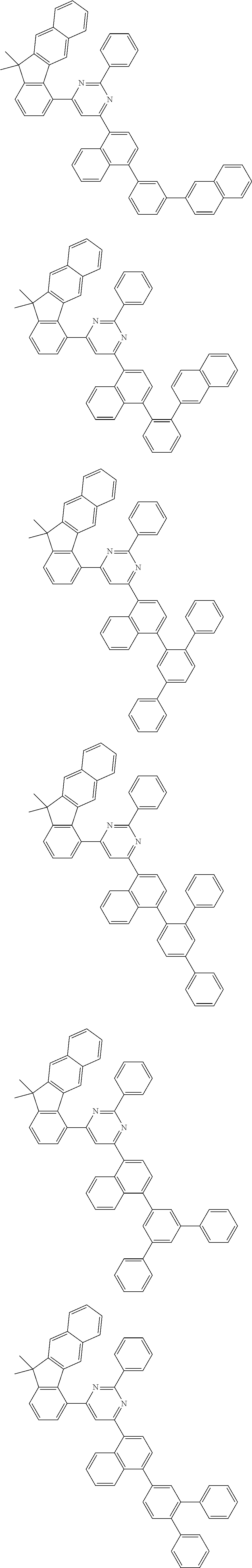

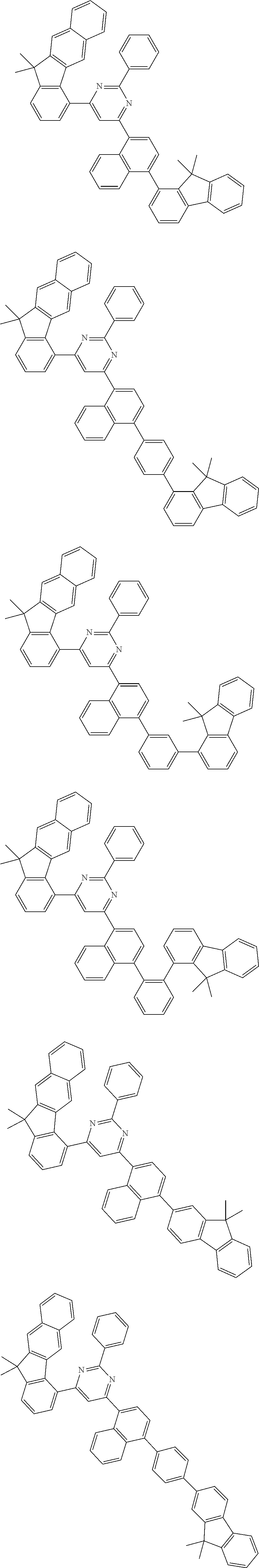

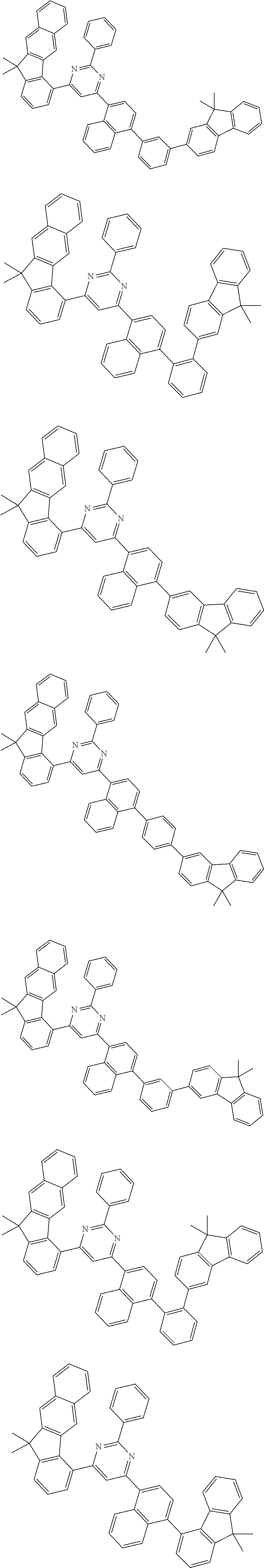

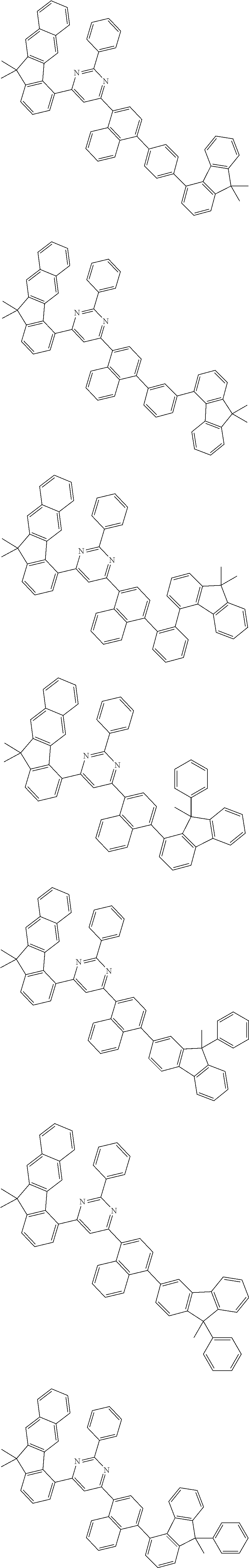

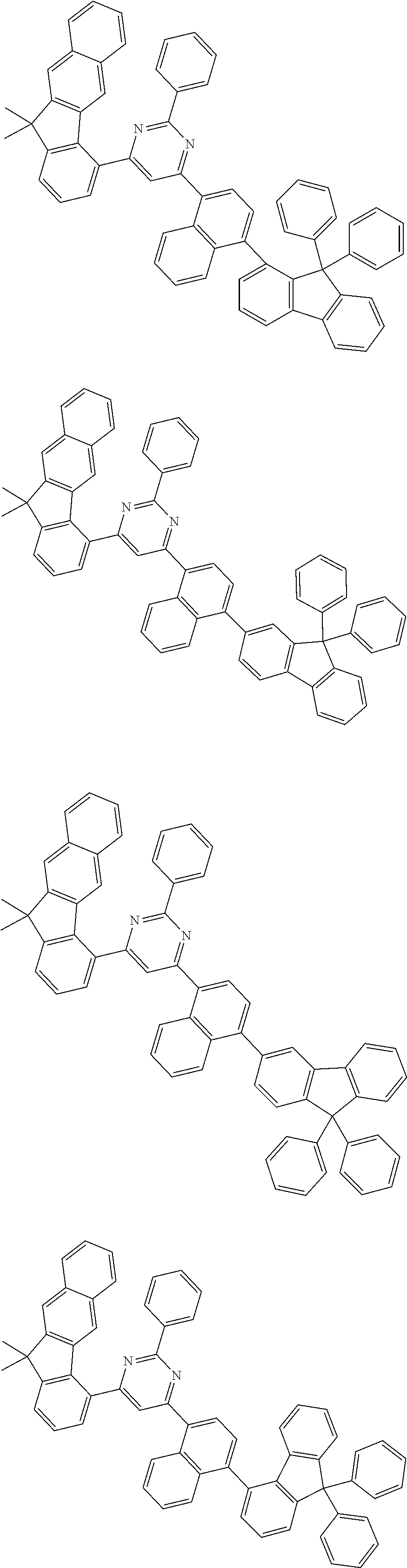 [SMILES: CC1(C)c2cc3ccccc3cc2-c2c(-c3cc(-c4ccc(-c5cccc6c5C(c5ccccc5)(c5ccccc5)c5ccccc5-6)c5ccccc45)nc(-c4ccccc4)n3)cccc21 ; CC1(C)c2cc3ccccc3cc2-c2c(-c3cc(-c4ccc(-c5ccc6c(c5)C(c5ccccc5)(c5ccccc5)c5ccccc5-6)c5ccccc45)nc(-c4ccccc4)n3)cccc21 ; CC1(C)c2cc3ccccc3cc2-c2c(-c3cc(-c4ccc(-c5ccc6c(c5)-c5ccccc5C6(c5ccccc5)c5ccccc5)c5ccccc45)nc(-c4ccccc4)n3)cccc21 ; CC1(C)c2cc3ccccc3cc2-c2c(-c3cc(-c4ccc(-c5cccc6c5-c5ccccc5C6(c5ccccc5)c5ccccc5)c5ccccc45)nc(-c4ccccc4)n3)cccc21]

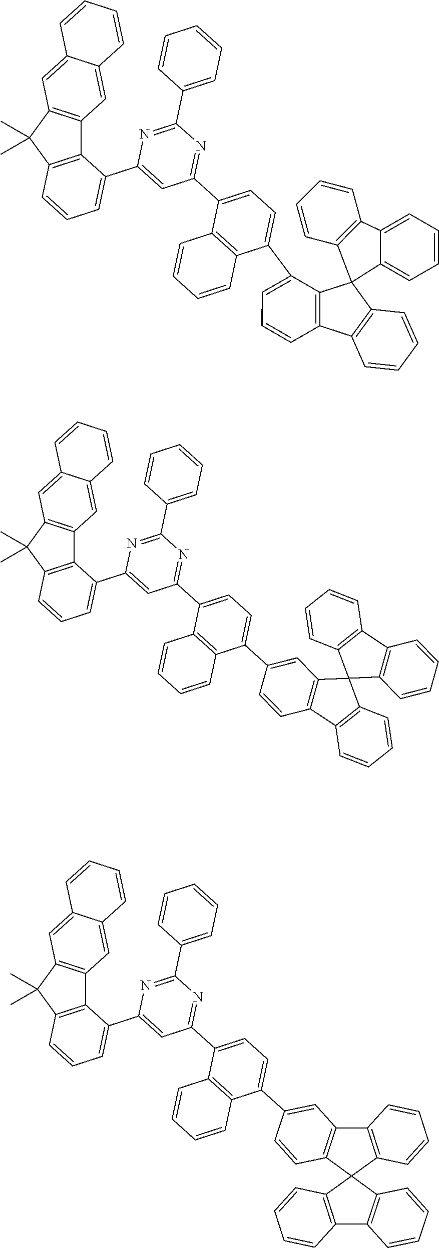 [SMILES: CC1(C)c2cc3ccccc3cc2-c2c(-c3cc(-c4ccc(-c5cccc6c5C5(c7ccccc7-c7ccccc75)c5ccccc5-6)c5ccccc45)nc(-c4ccccc4)n3)cccc21 ; CC1(C)c2cc3ccccc3cc2-c2c(-c3cc(-c4ccc(-c5ccc6c(c5)C5(c7ccccc7-c7ccccc75)c5ccccc5-6)c5ccccc45)nc(-c4ccccc4)n3)cccc21 ; CC1(C)c2cc3ccccc3cc2-c2c(-c3cc(-c4ccc(-c5ccc6c(c5)-c5ccccc5C65c6ccccc6-c6ccccc65)c5ccccc45)nc(-c4ccccc4)n3)cccc21]

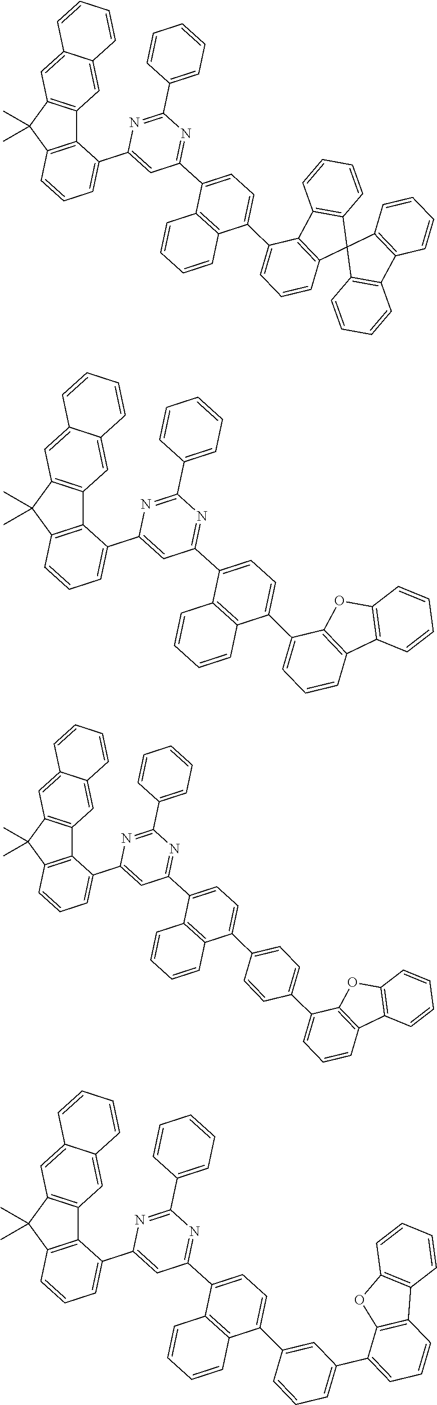 [SMILES: CC1(C)c2cc3ccccc3cc2-c2c(-c3cc(-c4ccc(-c5cccc6c5-c5ccccc5C65c6ccccc6-c6ccccc65)c5ccccc45)nc(-c4ccccc4)n3)cccc21 ; CC1(C)c2cc3ccccc3cc2-c2c(-c3cc(-c4ccc(-c5cccc6c5oc5ccccc56)c5ccccc45)nc(-c4ccccc4)n3)cccc21 ; CC1(C)c2cc3ccccc3cc2-c2c(-c3cc(-c4ccc(-c5ccc(-c6cccc7c6oc6ccccc67)cc5)c5ccccc45)nc(-c4ccccc4)n3)cccc21 ; CC1(C)c2cc3ccccc3cc2-c2c(-c3cc(-c4ccc(-c5cccc(-c6cccc7c6oc6ccccc67)c5)c5ccccc45)nc(-c4ccccc4)n3)cccc21]

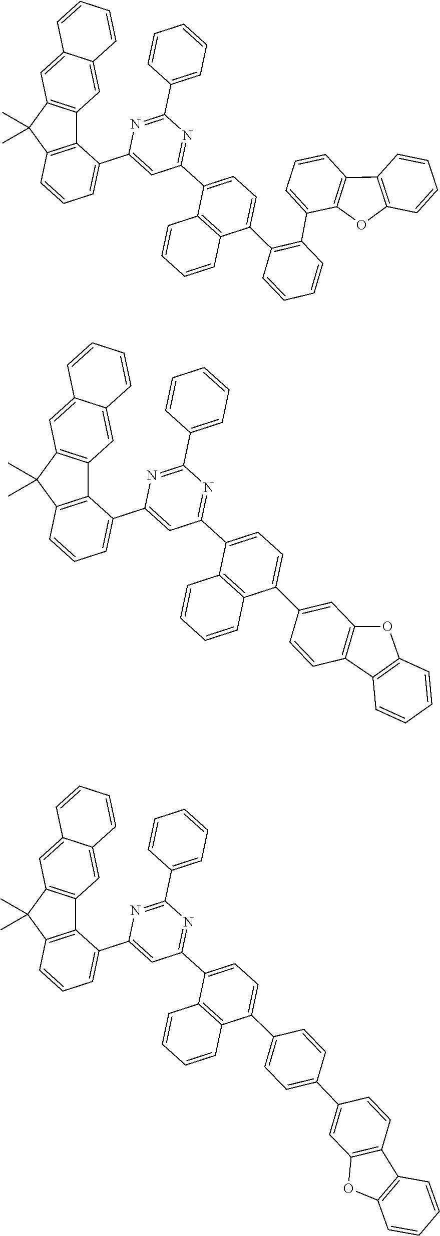 [SMILES: CC1(C)c2cc3ccccc3cc2-c2c(-c3cc(-c4ccc(-c5ccccc5-c5cccc6c5oc5ccccc56)c5ccccc45)nc(-c4ccccc4)n3)cccc21 ; CC1(C)c2cc3ccccc3cc2-c2c(-c3cc(-c4ccc(-c5ccc6c(c5)oc5ccccc56)c5ccccc45)nc(-c4ccccc4)n3)cccc21 ; CC1(C)c2cc3ccccc3cc2-c2c(-c3cc(-c4ccc(-c5ccc(-c6ccc7c(c6)oc6ccccc67)cc5)c5ccccc45)nc(-c4ccccc4)n3)cccc21]

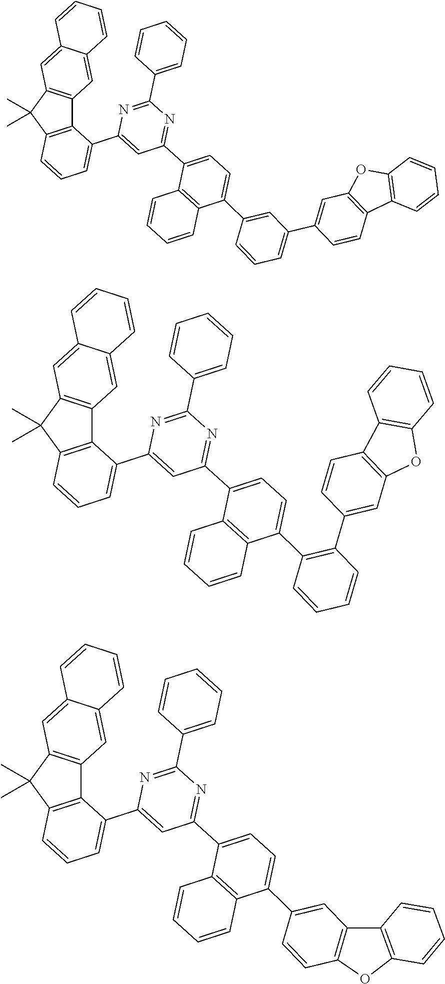 [SMILES: CC1(C)c2cc3ccccc3cc2-c2c(-c3cc(-c4ccc(-c5cccc(-c6ccc7c(c6)oc6ccccc67)c5)c5ccccc45)nc(-c4ccccc4)n3)cccc21 ; CC1(C)c2cc3ccccc3cc2-c2c(-c3cc(-c4ccc(-c5ccccc5-c5ccc6c(c5)oc5ccccc56)c5ccccc45)nc(-c4ccccc4)n3)cccc21 ; CC1(C)c2cc3ccccc3cc2-c2c(-c3cc(-c4ccc(-c5ccc6oc7ccccc7c6c5)c5ccccc45)nc(-c4ccccc4)n3)cccc21]

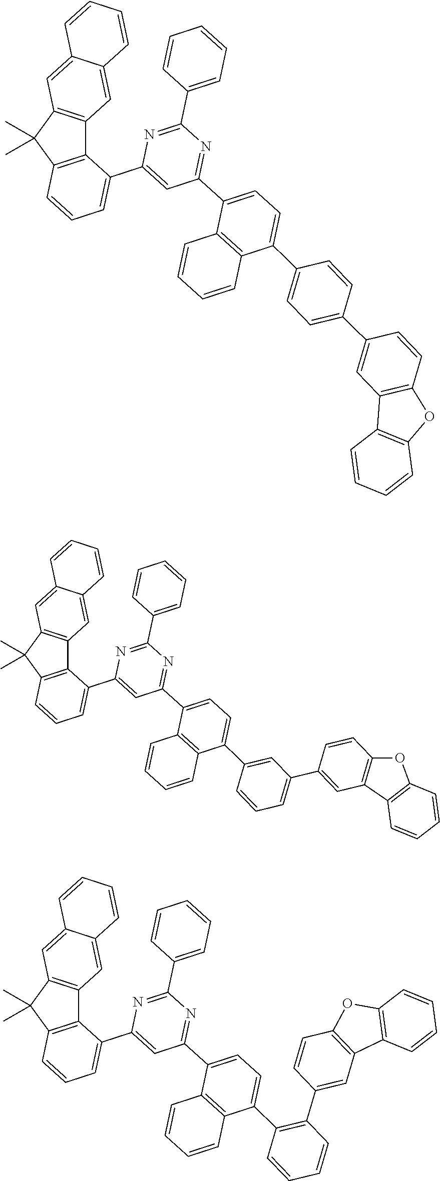 [SMILES: CC1(C)c2cc3ccccc3cc2-c2c(-c3cc(-c4ccc(-c5ccc(-c6ccc7oc8ccccc8c7c6)cc5)c5ccccc45)nc(-c4ccccc4)n3)cccc21 ; CC1(C)c2cc3ccccc3cc2-c2c(-c3cc(-c4ccc(-c5cccc(-c6ccc7oc8ccccc8c7c6)c5)c5ccccc45)nc(-c4ccccc4)n3)cccc21 ; CC1(C)c2cc3ccccc3cc2-c2c(-c3cc(-c4ccc(-c5ccccc5-c5ccc6oc7ccccc7c6c5)c5ccccc45)nc(-c4ccccc4)n3)cccc21]

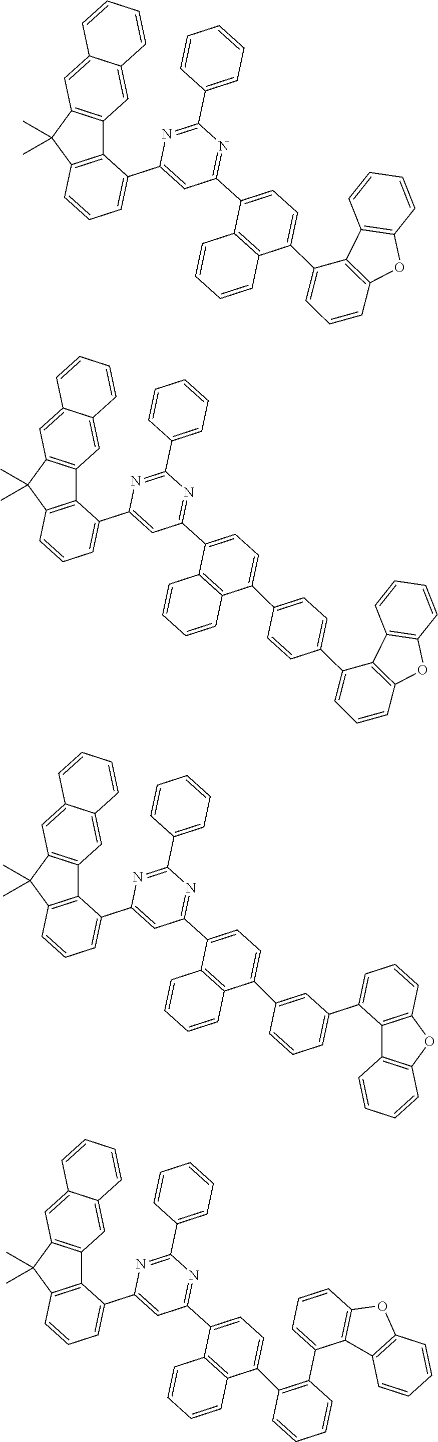 [SMILES: CC1(C)c2cc3ccccc3cc2-c2c(-c3cc(-c4ccc(-c5cccc6oc7ccccc7c56)c5ccccc45)nc(-c4ccccc4)n3)cccc21 ; CC1(C)c2cc3ccccc3cc2-c2c(-c3cc(-c4ccc(-c5ccc(-c6cccc7oc8ccccc8c67)cc5)c5ccccc45)nc(-c4ccccc4)n3)cccc21 ; CC1(C)c2cc3ccccc3cc2-c2c(-c3cc(-c4ccc(-c5cccc(-c6cccc7oc8ccccc8c67)c5)c5ccccc45)nc(-c4ccccc4)n3)cccc21 ; CC1(C)c2cc3ccccc3cc2-c2c(-c3cc(-c4ccc(-c5ccccc5-c5cccc6oc7ccccc7c56)c5ccccc45)nc(-c4ccccc4)n3)cccc21]

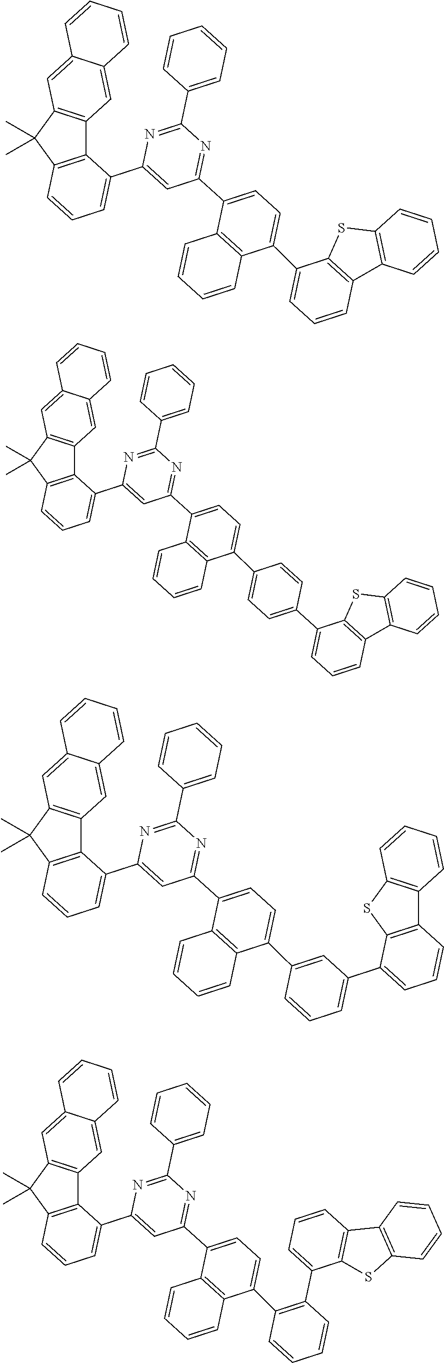 [SMILES: CC1(C)c2cc3ccccc3cc2-c2c(-c3cc(-c4ccc(-c5cccc6c5sc5ccccc56)c5ccccc45)nc(-c4ccccc4)n3)cccc21 ; CC1(C)c2cc3ccccc3cc2-c2c(-c3cc(-c4ccc(-c5ccc(-c6cccc7c6sc6ccccc67)cc5)c5ccccc45)nc(-c4ccccc4)n3)cccc21 ; CC1(C)c2cc3ccccc3cc2-c2c(-c3cc(-c4ccc(-c5cccc(-c6cccc7c6sc6ccccc67)c5)c5ccccc45)nc(-c4ccccc4)n3)cccc21 ; CC1(C)c2cc3ccccc3cc2-c2c(-c3cc(-c4ccc(-c5ccccc5-c5cccc6c5sc5ccccc56)c5ccccc45)nc(-c4ccccc4)n3)cccc21]

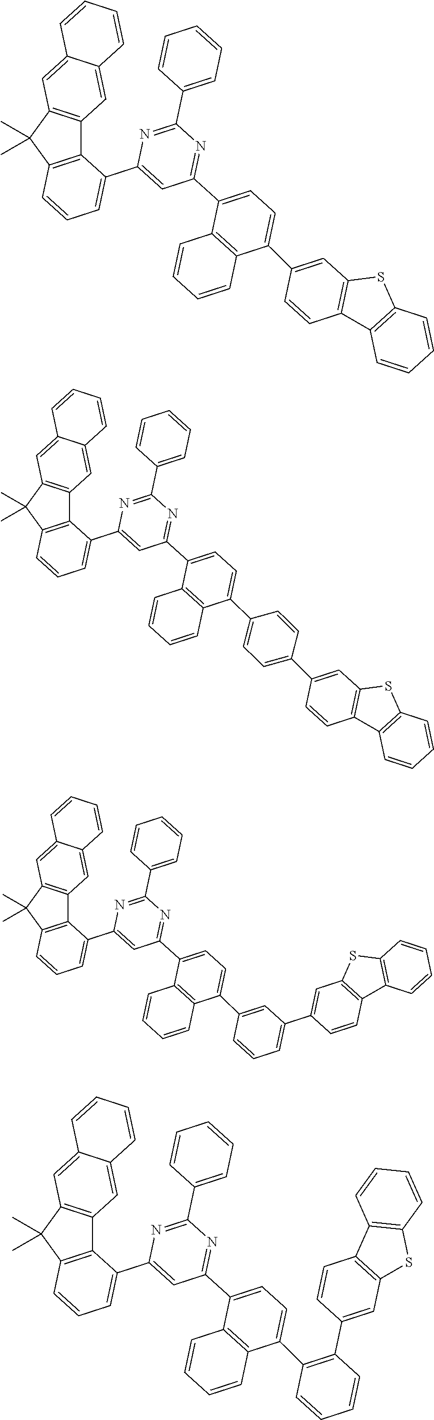 [SMILES: CC1(C)c2cc3ccccc3cc2-c2c(-c3cc(-c4ccc(-c5ccc6c(c5)sc5ccccc56)c5ccccc45)nc(-c4ccccc4)n3)cccc21 ; CC1(C)c2cc3ccccc3cc2-c2c(-c3cc(-c4ccc(-c5ccc(-c6ccc7c(c6)sc6ccccc67)cc5)c5ccccc45)nc(-c4ccccc4)n3)cccc21 ; CC1(C)c2cc3ccccc3cc2-c2c(-c3cc(-c4ccc(-c5cccc(-c6ccc7c(c6)sc6ccccc67)c5)c5ccccc45)nc(-c4ccccc4)n3)cccc21 ; CC1(C)c2cc3ccccc3cc2-c2c(-c3cc(-c4ccc(-c5ccccc5-c5ccc6c(c5)sc5ccccc56)c5ccccc45)nc(-c4ccccc4)n3)cccc21]

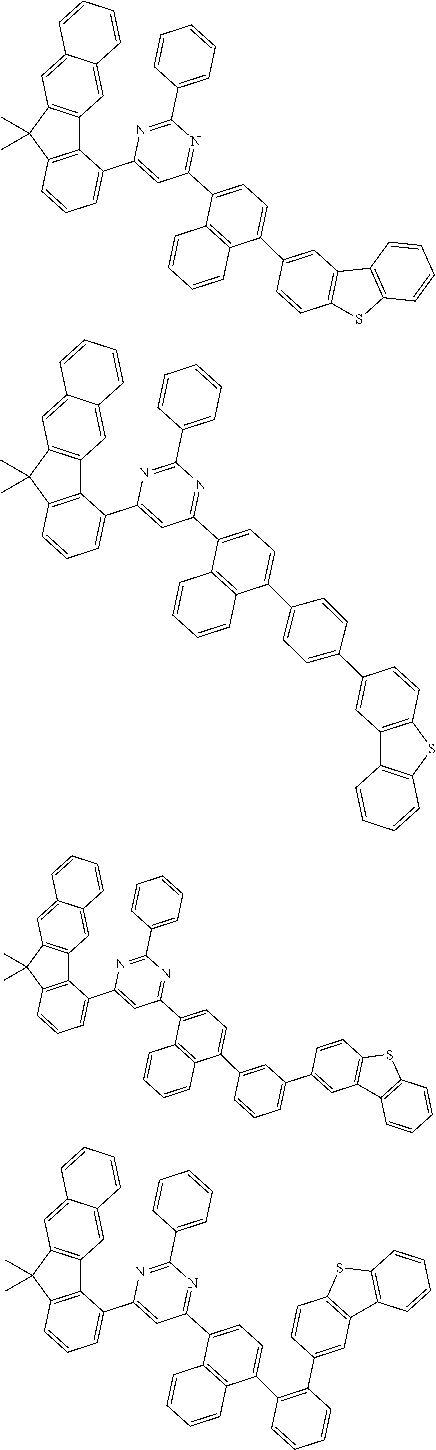 [SMILES: CC1(C)c2cc3ccccc3cc2-c2c(-c3cc(-c4ccc(-c5ccc6sc7ccccc7c6c5)c5ccccc45)nc(-c4ccccc4)n3)cccc21 ; CC1(C)c2cc3ccccc3cc2-c2c(-c3cc(-c4ccc(-c5ccc(-c6ccc7sc8ccccc8c7c6)cc5)c5ccccc45)nc(-c4ccccc4)n3)cccc21 ; CC1(C)c2cc3ccccc3cc2-c2c(-c3cc(-c4ccc(-c5cccc(-c6ccc7sc8ccccc8c7c6)c5)c5ccccc45)nc(-c4ccccc4)n3)cccc21 ; CC1(C)c2cc3ccccc3cc2-c2c(-c3cc(-c4ccc(-c5ccccc5-c5ccc6sc7ccccc7c6c5)c5ccccc45)nc(-c4ccccc4)n3)cccc21]

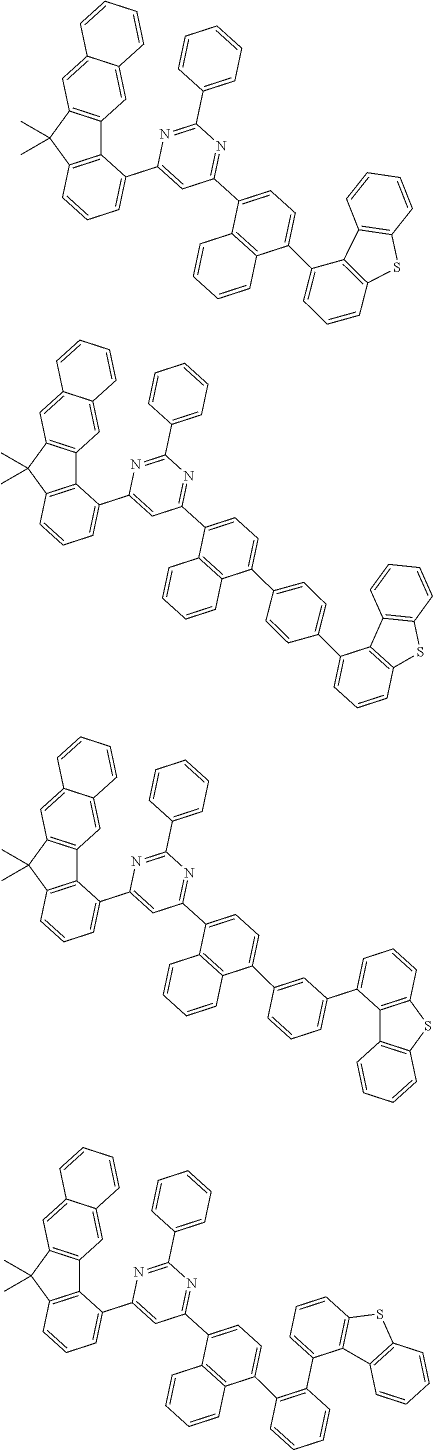 [SMILES: CC1(C)c2cc3ccccc3cc2-c2c(-c3cc(-c4ccc(-c5cccc6sc7ccccc7c56)c5ccccc45)nc(-c4ccccc4)n3)cccc21 ; CC1(C)c2cc3ccccc3cc2-c2c(-c3cc(-c4ccc(-c5ccc(-c6cccc7sc8ccccc8c67)cc5)c5ccccc45)nc(-c4ccccc4)n3)cccc21 ; CC1(C)c2cc3ccccc3cc2-c2c(-c3cc(-c4ccc(-c5cccc(-c6cccc7sc8ccccc8c67)c5)c5ccccc45)nc(-c4ccccc4)n3)cccc21 ; CC1(C)c2cc3ccccc3cc2-c2c(-c3cc(-c4ccc(-c5ccccc5-c5cccc6sc7ccccc7c56)c5ccccc45)nc(-c4ccccc4)n3)cccc21]

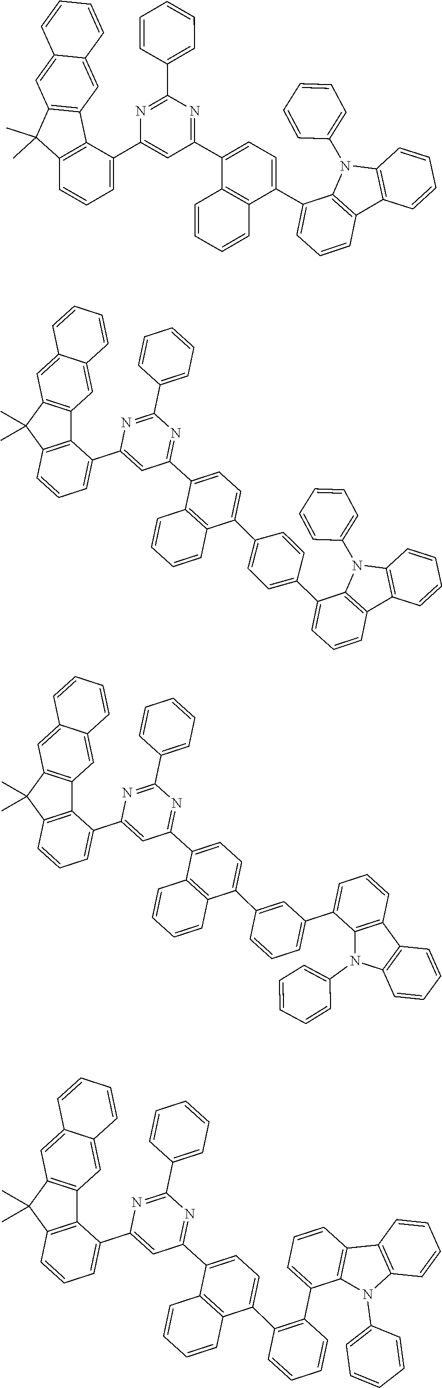 [SMILES: CC1(C)c2cc3ccccc3cc2-c2c(-c3cc(-c4ccc(-c5cccc6c7ccccc7n(-c7ccccc7)c56)c5ccccc45)nc(-c4ccccc4)n3)cccc21 ; CC1(C)c2cc3ccccc3cc2-c2c(-c3cc(-c4ccc(-c5ccc(-c6cccc7c8ccccc8n(-c8ccccc8)c67)cc5)c5ccccc45)nc(-c4ccccc4)n3)cccc21 ; CC1(C)c2cc3ccccc3cc2-c2c(-c3cc(-c4ccc(-c5cccc(-c6cccc7c8ccccc8n(-c8ccccc8)c67)c5)c5ccccc45)nc(-c4ccccc4)n3)cccc21 ; CC1(C)c2cc3ccccc3cc2-c2c(-c3cc(-c4ccc(-c5ccccc5-c5cccc6c7ccccc7n(-c7ccccc7)c56)c5ccccc45)nc(-c4ccccc4)n3)cccc21]

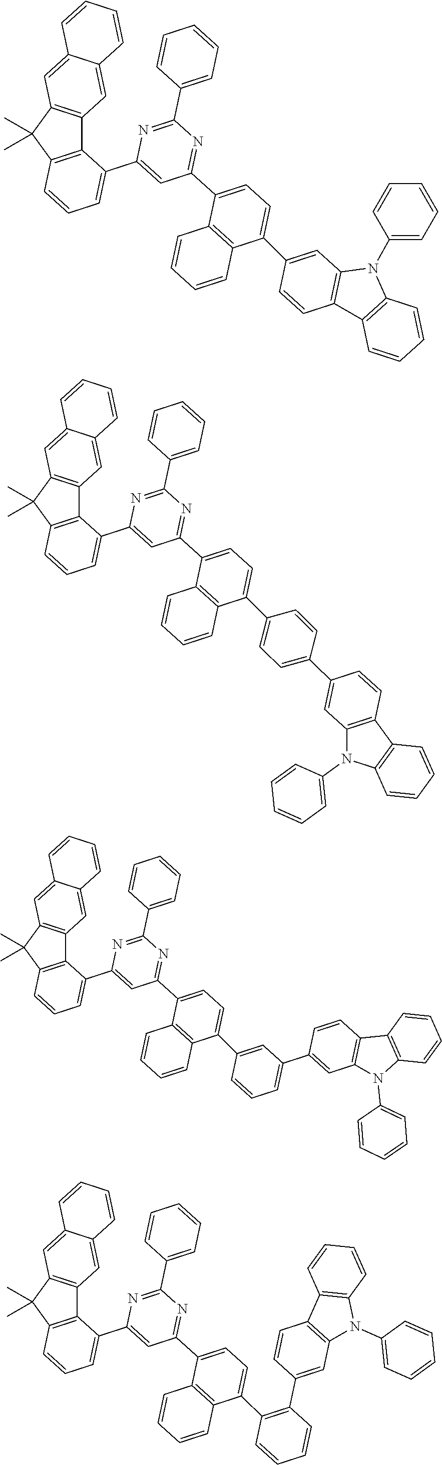 [SMILES: CC1(C)c2cc3ccccc3cc2-c2c(-c3cc(-c4ccc(-c5ccc6c7ccccc7n(-c7ccccc7)c6c5)c5ccccc45)nc(-c4ccccc4)n3)cccc21 ; CC1(C)c2cc3ccccc3cc2-c2c(-c3cc(-c4ccc(-c5ccc(-c6ccc7c8ccccc8n(-c8ccccc8)c7c6)cc5)c5ccccc45)nc(-c4ccccc4)n3)cccc21 ; CC1(C)c2cc3ccccc3cc2-c2c(-c3cc(-c4ccc(-c5cccc(-c6ccc7c8ccccc8n(-c8ccccc8)c7c6)c5)c5ccccc45)nc(-c4ccccc4)n3)cccc21 ; CC1(C)c2cc3ccccc3cc2-c2c(-c3cc(-c4ccc(-c5ccccc5-c5ccc6c7ccccc7n(-c7ccccc7)c6c5)c5ccccc45)nc(-c4ccccc4)n3)cccc21]

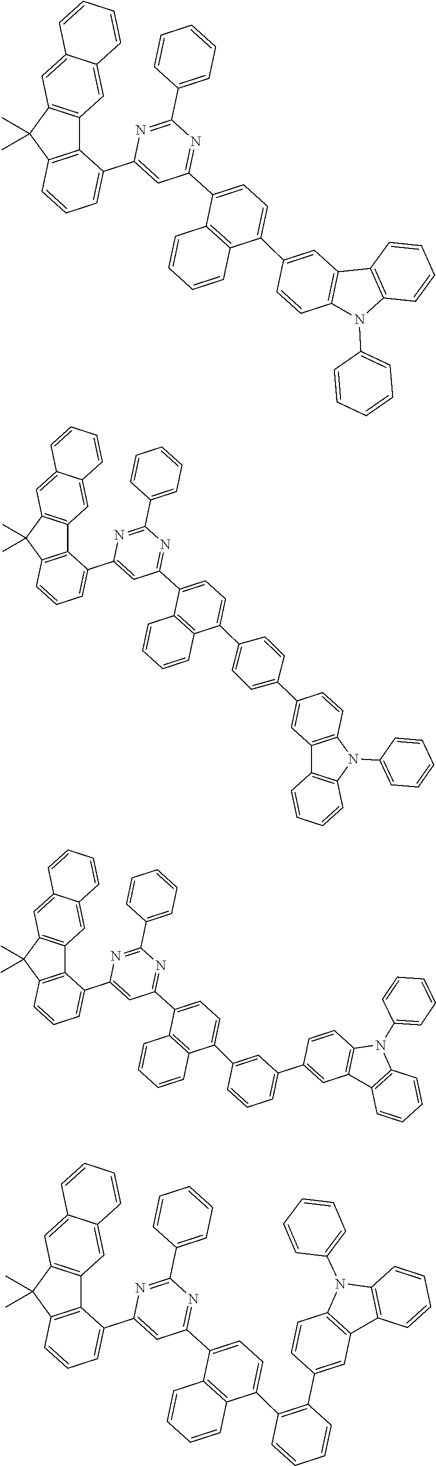 [SMILES: CC1(C)c2cc3ccccc3cc2-c2c(-c3cc(-c4ccc(-c5ccc6c(c5)c5ccccc5n6-c5ccccc5)c5ccccc45)nc(-c4ccccc4)n3)cccc21 ; CC1(C)c2cc3ccccc3cc2-c2c(-c3cc(-c4ccc(-c5ccc(-c6ccc7c(c6)c6ccccc6n7-c6ccccc6)cc5)c5ccccc45)nc(-c4ccccc4)n3)cccc21 ; CC1(C)c2cc3ccccc3cc2-c2c(-c3cc(-c4ccc(-c5cccc(-c6ccc7c(c6)c6ccccc6n7-c6ccccc6)c5)c5ccccc45)nc(-c4ccccc4)n3)cccc21 ; CC1(C)c2cc3ccccc3cc2-c2c(-c3cc(-c4ccc(-c5ccccc5-c5ccc6c(c5)c5ccccc5n6-c5ccccc5)c5ccccc45)nc(-c4ccccc4)n3)cccc21]

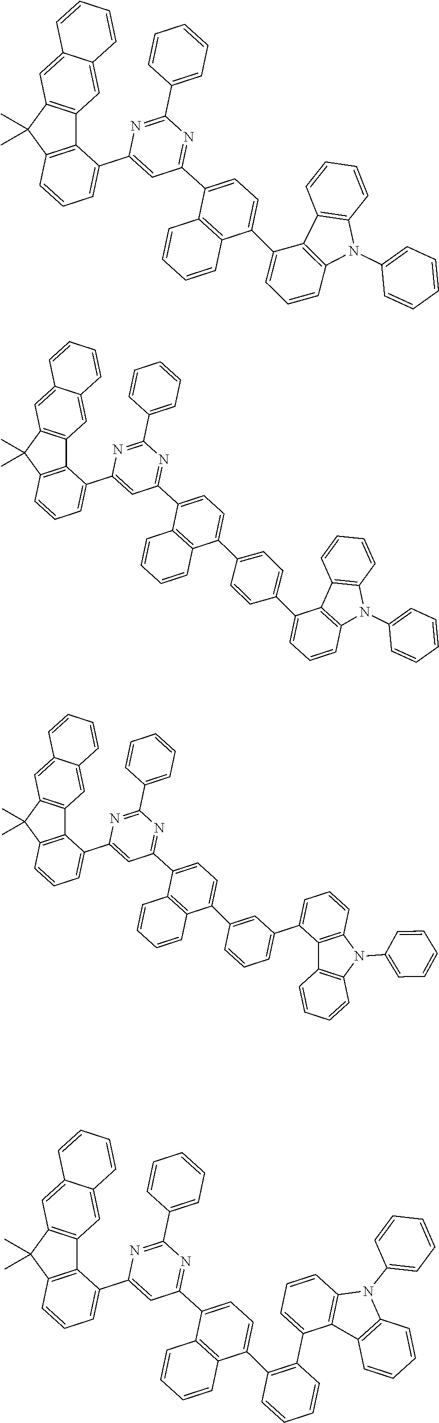 [SMILES: CC1(C)c2cc3ccccc3cc2-c2c(-c3cc(-c4ccc(-c5cccc6c5c5ccccc5n6-c5ccccc5)c5ccccc45)nc(-c4ccccc4)n3)cccc21 ; CC1(C)c2cc3ccccc3cc2-c2c(-c3cc(-c4ccc(-c5ccc(-c6cccc7c6c6ccccc6n7-c6ccccc6)cc5)c5ccccc45)nc(-c4ccccc4)n3)cccc21 ; CC1(C)c2cc3ccccc3cc2-c2c(-c3cc(-c4ccc(-c5cccc(-c6cccc7c6c6ccccc6n7-c6ccccc6)c5)c5ccccc45)nc(-c4ccccc4)n3)cccc21 ; CC1(C)c2cc3ccccc3cc2-c2c(-c3cc(-c4ccc(-c5ccccc5-c5cccc6c5c5ccccc5n6-c5ccccc5)c5ccccc45)nc(-c4ccccc4)n3)cccc21]

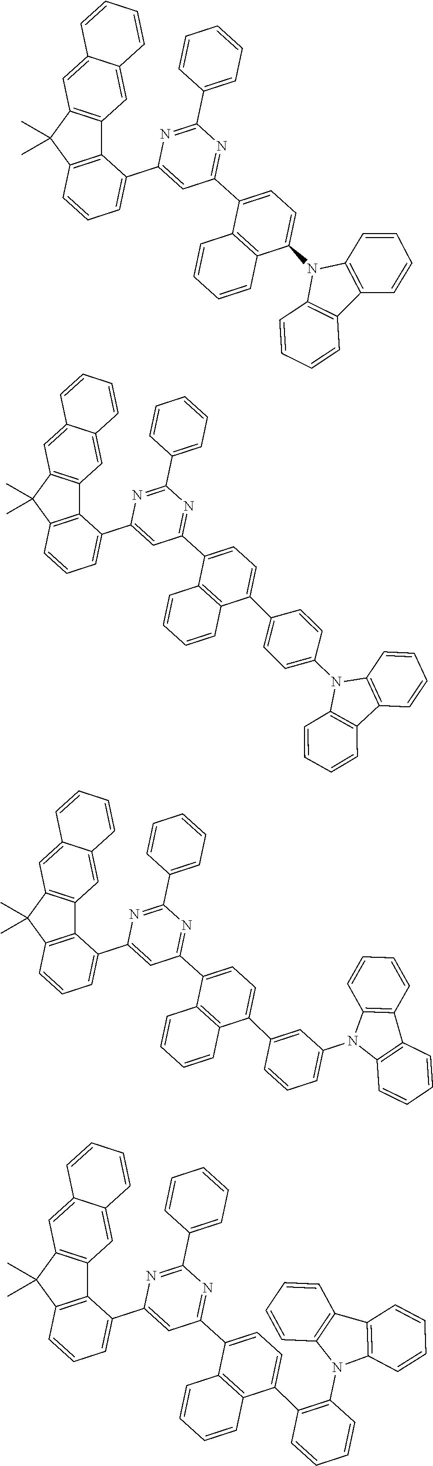

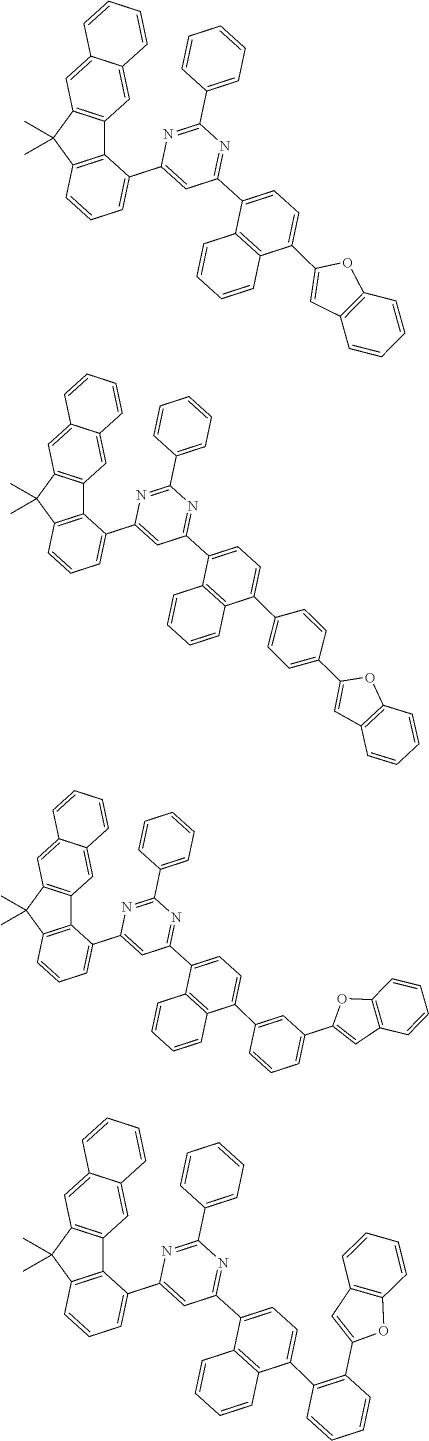 [SMILES: CC1(C)c2cc3ccccc3cc2-c2c(-c3cc(-c4ccc(-c5cc6ccccc6o5)c5ccccc45)nc(-c4ccccc4)n3)cccc21 ; CC1(C)c2cc3ccccc3cc2-c2c(-c3cc(-c4ccc(-c5ccc(-c6cc7ccccc7o6)cc5)c5ccccc45)nc(-c4ccccc4)n3)cccc21 ; CC1(C)c2cc3ccccc3cc2-c2c(-c3cc(-c4ccc(-c5cccc(-c6cc7ccccc7o6)c5)c5ccccc45)nc(-c4ccccc4)n3)cccc21 ; CC1(C)c2cc3ccccc3cc2-c2c(-c3cc(-c4ccc(-c5ccccc5-c5cc6ccccc6o5)c5ccccc45)nc(-c4ccccc4)n3)cccc21]

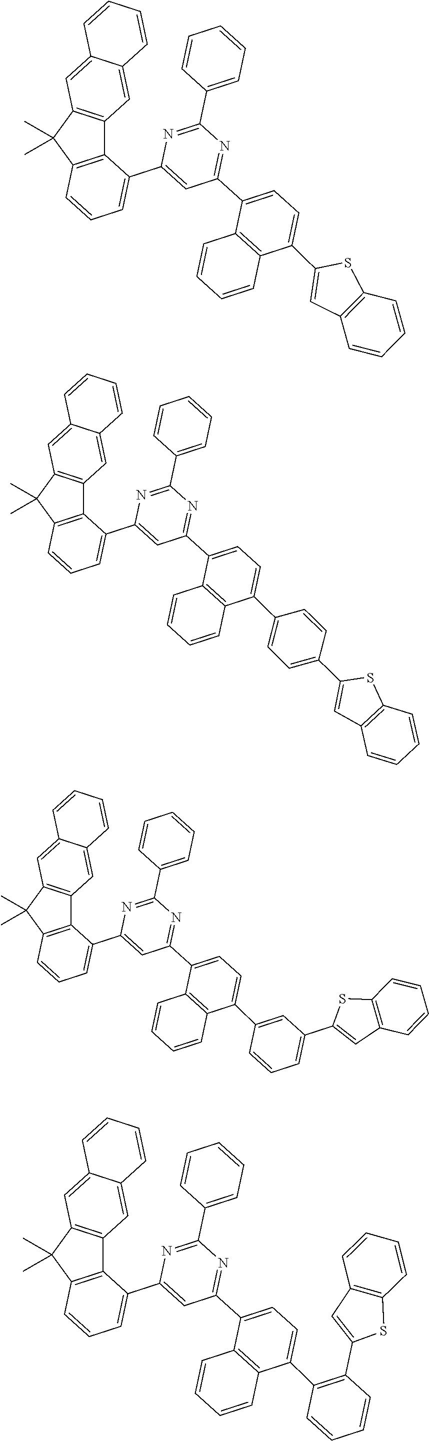 [SMILES: CC1(C)c2cc3ccccc3cc2-c2c(-c3cc(-c4ccc(-c5cc6ccccc6s5)c5ccccc45)nc(-c4ccccc4)n3)cccc21 ; CC1(C)c2cc3ccccc3cc2-c2c(-c3cc(-c4ccc(-c5ccc(-c6cc7ccccc7s6)cc5)c5ccccc45)nc(-c4ccccc4)n3)cccc21 ; CC1(C)c2cc3ccccc3cc2-c2c(-c3cc(-c4ccc(-c5cccc(-c6cc7ccccc7s6)c5)c5ccccc45)nc(-c4ccccc4)n3)cccc21 ; CC1(C)c2cc3ccccc3cc2-c2c(-c3cc(-c4ccc(-c5ccccc5-c5cc6ccccc6s5)c5ccccc45)nc(-c4ccccc4)n3)cccc21]

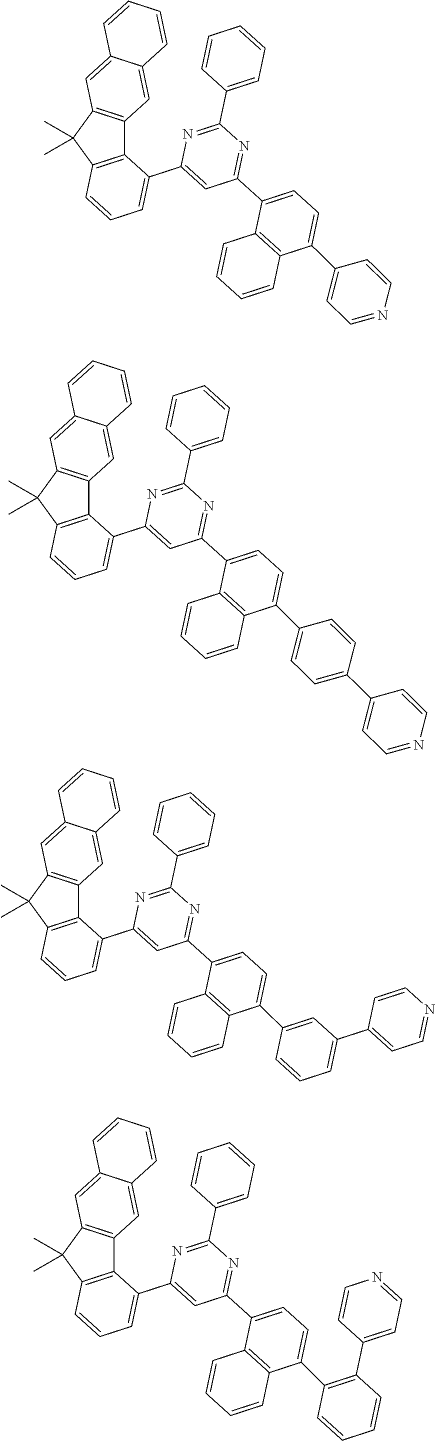 [SMILES: CC1(C)c2cc3ccccc3cc2-c2c(-c3cc(-c4ccc(-c5ccncc5)c5ccccc45)nc(-c4ccccc4)n3)cccc21 ; CC1(C)c2cc3ccccc3cc2-c2c(-c3cc(-c4ccc(-c5ccc(-c6ccncc6)cc5)c5ccccc45)nc(-c4ccccc4)n3)cccc21 ; CC1(C)c2cc3ccccc3cc2-c2c(-c3cc(-c4ccc(-c5cccc(-c6ccncc6)c5)c5ccccc45)nc(-c4ccccc4)n3)cccc21 ; CC1(C)c2cc3ccccc3cc2-c2c(-c3cc(-c4ccc(-c5ccccc5-c5ccncc5)c5ccccc45)nc(-c4ccccc4)n3)cccc21]

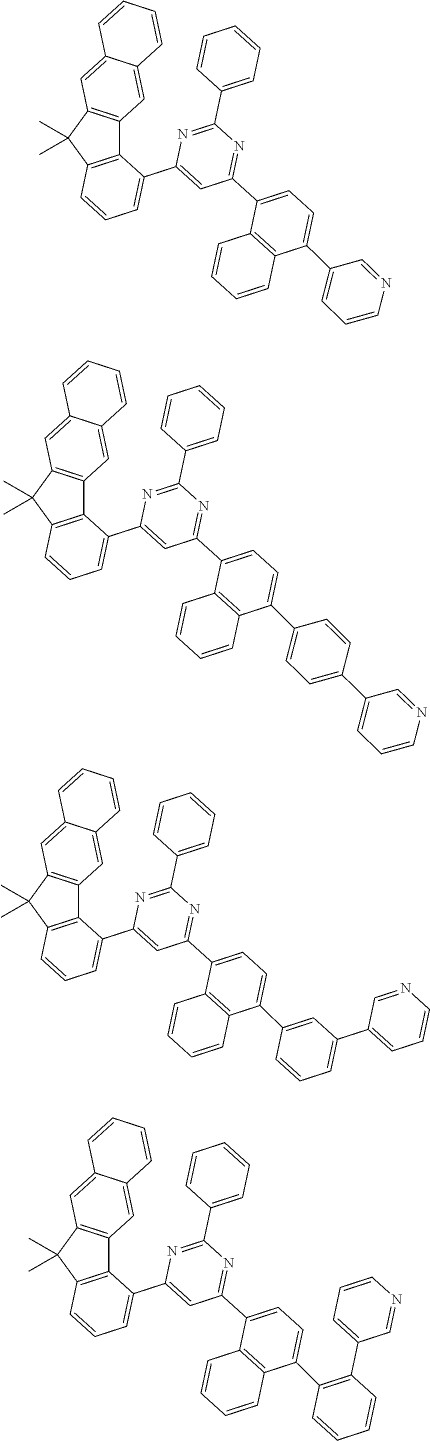 [SMILES: CC1(C)c2cc3ccccc3cc2-c2c(-c3cc(-c4ccc(-c5cccnc5)c5ccccc45)nc(-c4ccccc4)n3)cccc21 ; CC1(C)c2cc3ccccc3cc2-c2c(-c3cc(-c4ccc(-c5ccc(-c6cccnc6)cc5)c5ccccc45)nc(-c4ccccc4)n3)cccc21 ; CC1(C)c2cc3ccccc3cc2-c2c(-c3cc(-c4ccc(-c5cccc(-c6cccnc6)c5)c5ccccc45)nc(-c4ccccc4)n3)cccc21 ; CC1(C)c2cc3ccccc3cc2-c2c(-c3cc(-c4ccc(-c5ccccc5-c5cccnc5)c5ccccc45)nc(-c4ccccc4)n3)cccc21]

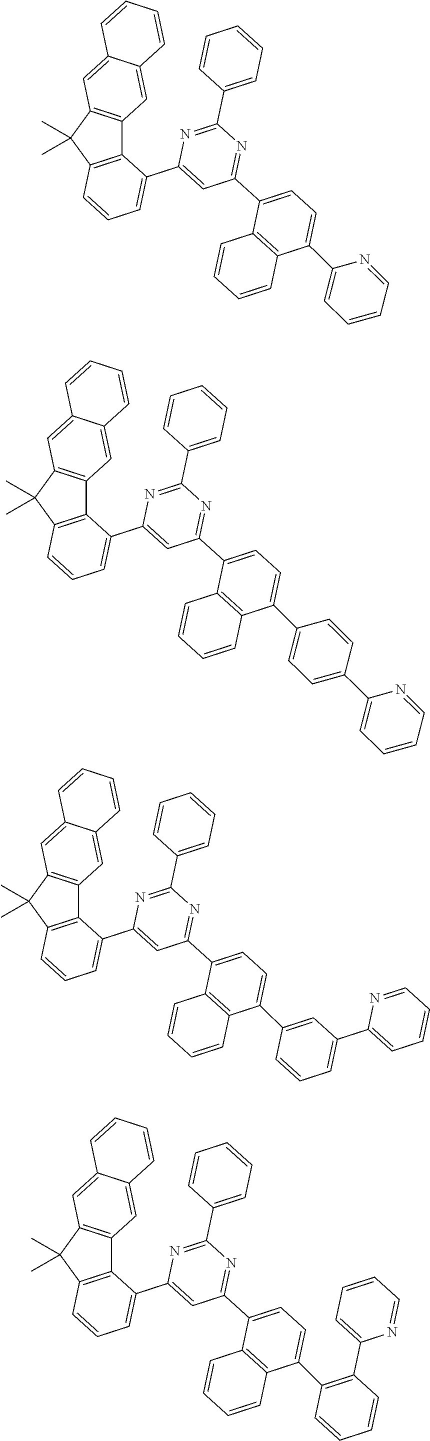 [SMILES: CC1(C)c2cc3ccccc3cc2-c2c(-c3cc(-c4ccc(-c5ccccn5)c5ccccc45)nc(-c4ccccc4)n3)cccc21 ; CC1(C)c2cc3ccccc3cc2-c2c(-c3cc(-c4ccc(-c5ccc(-c6ccccn6)cc5)c5ccccc45)nc(-c4ccccc4)n3)cccc21 ; CC1(C)c2cc3ccccc3cc2-c2c(-c3cc(-c4ccc(-c5cccc(-c6ccccn6)c5)c5ccccc45)nc(-c4ccccc4)n3)cccc21 ; CC1(C)c2cc3ccccc3cc2-c2c(-c3cc(-c4ccc(-c5ccccc5-c5ccccn5)c5ccccc45)nc(-c4ccccc4)n3)cccc21]

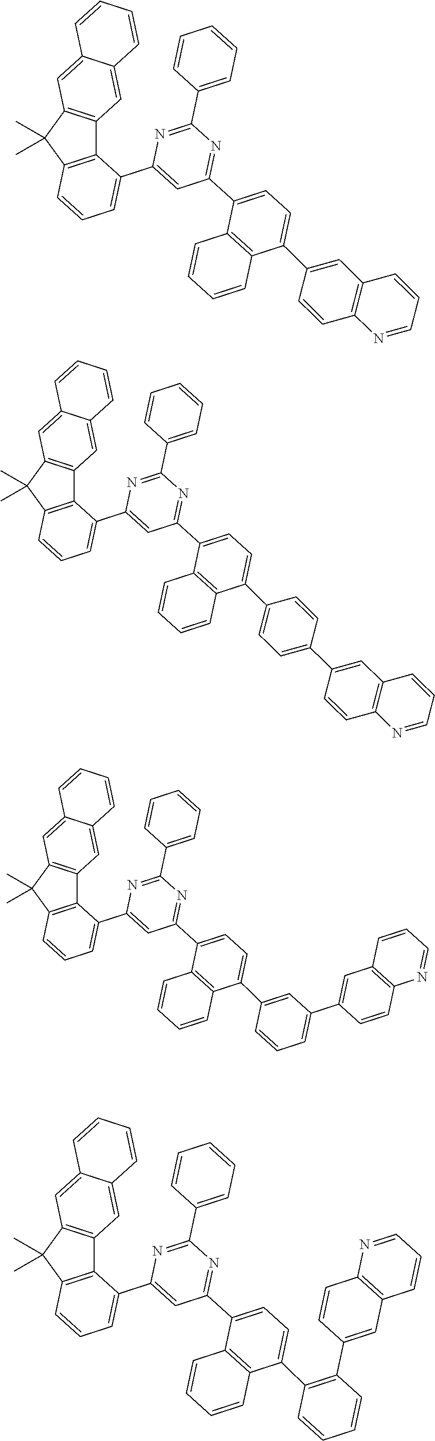 [SMILES: CC1(C)c2cc3ccccc3cc2-c2c(-c3cc(-c4ccc(-c5ccc6ncccc6c5)c5ccccc45)nc(-c4ccccc4)n3)cccc21 ; CC1(C)c2cc3ccccc3cc2-c2c(-c3cc(-c4ccc(-c5ccc(-c6ccc7ncccc7c6)cc5)c5ccccc45)nc(-c4ccccc4)n3)cccc21 ; CC1(C)c2cc3ccccc3cc2-c2c(-c3cc(-c4ccc(-c5cccc(-c6ccc7ncccc7c6)c5)c5ccccc45)nc(-c4ccccc4)n3)cccc21 ; CC1(C)c2cc3ccccc3cc2-c2c(-c3cc(-c4ccc(-c5ccccc5-c5ccc6ncccc6c5)c5ccccc45)nc(-c4ccccc4)n3)cccc21]

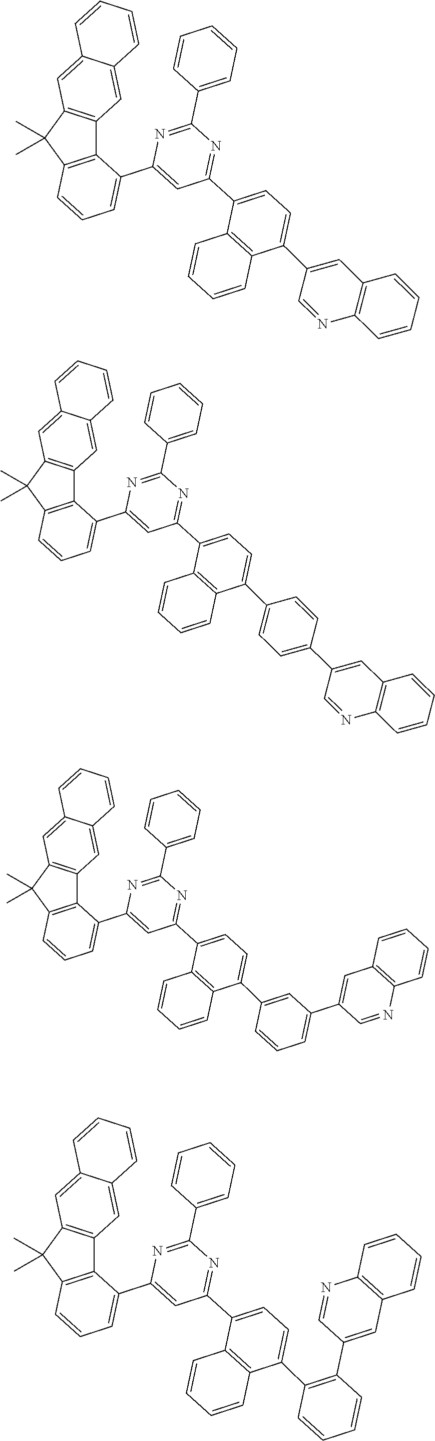 [SMILES: CC1(C)c2cc3ccccc3cc2-c2c(-c3cc(-c4ccc(-c5cnc6ccccc6c5)c5ccccc45)nc(-c4ccccc4)n3)cccc21 ; CC1(C)c2cc3ccccc3cc2-c2c(-c3cc(-c4ccc(-c5ccc(-c6cnc7ccccc7c6)cc5)c5ccccc45)nc(-c4ccccc4)n3)cccc21 ; CC1(C)c2cc3ccccc3cc2-c2c(-c3cc(-c4ccc(-c5cccc(-c6cnc7ccccc7c6)c5)c5ccccc45)nc(-c4ccccc4)n3)cccc21 ; CC1(C)c2cc3ccccc3cc2-c2c(-c3cc(-c4ccc(-c5ccccc5-c5cnc6ccccc6c5)c5ccccc45)nc(-c4ccccc4)n3)cccc21]

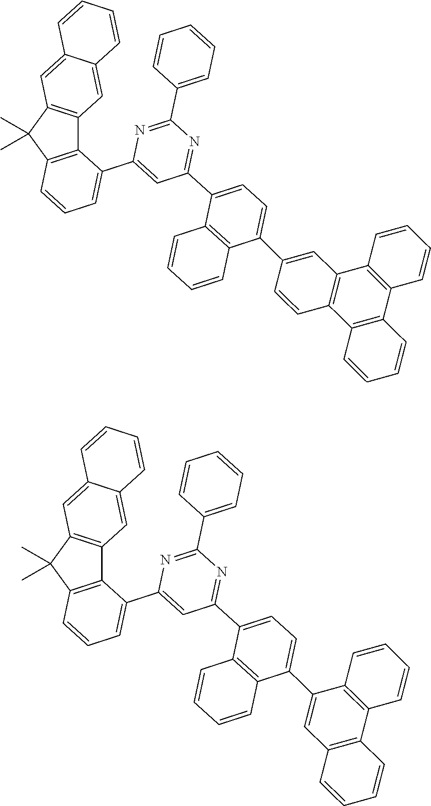 [SMILES: CC1(C)c2cc3ccccc3cc2-c2c(-c3cc(-c4ccc(-c5ccc6c7ccccc7c7ccccc7c6c5)c5ccccc45)nc(-c4ccccc4)n3)cccc21 ; CC1(C)c2cc3ccccc3cc2-c2c(-c3cc(-c4ccc(-c5cc6ccccc6c6ccccc56)c5ccccc45)nc(-c4ccccc4)n3)cccc21]

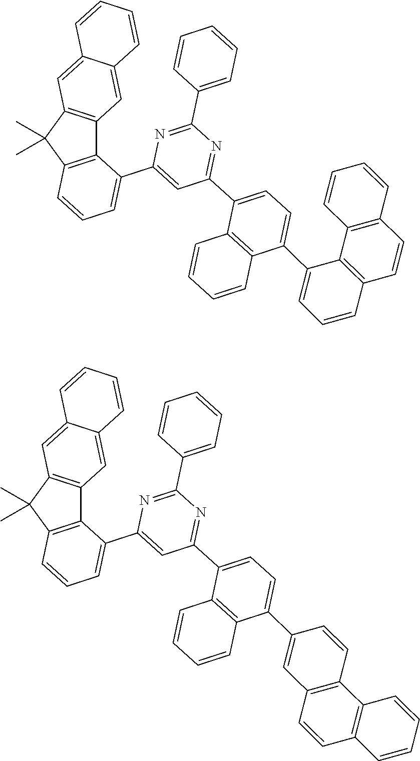 [SMILES: CC1(C)c2cc3ccccc3cc2-c2c(-c3cc(-c4ccc(-c5cccc6ccc7ccccc7c56)c5ccccc45)nc(-c4ccccc4)n3)cccc21 ; CC1(C)c2cc3ccccc3cc2-c2c(-c3cc(-c4ccc(-c5ccc6c(ccc7ccccc76)c5)c5ccccc45)nc(-c4ccccc4)n3)cccc21]

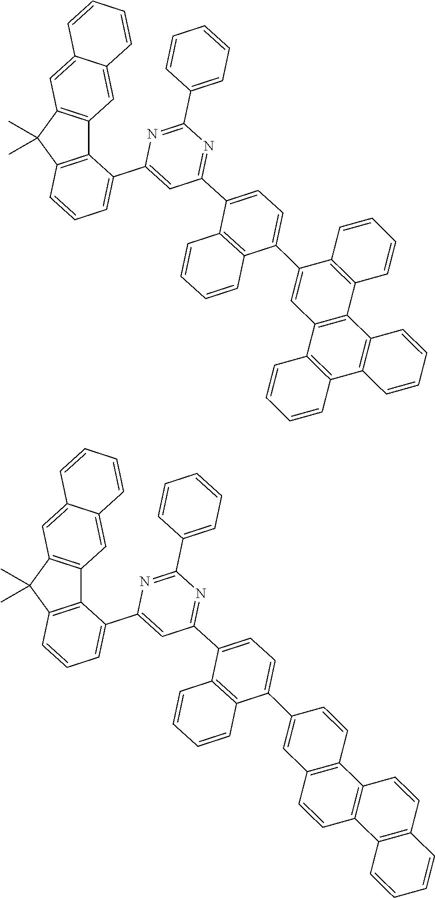 [SMILES: CC1(C)c2cc3ccccc3cc2-c2c(-c3cc(-c4ccc(-c5cc6c7ccccc7c7ccccc7c6c6ccccc56)c5ccccc45)nc(-c4ccccc4)n3)cccc21 ; CC1(C)c2cc3ccccc3cc2-c2c(-c3cc(-c4ccc(-c5ccc6c(ccc7c8ccccc8ccc67)c5)c5ccccc45)nc(-c4ccccc4)n3)cccc21]

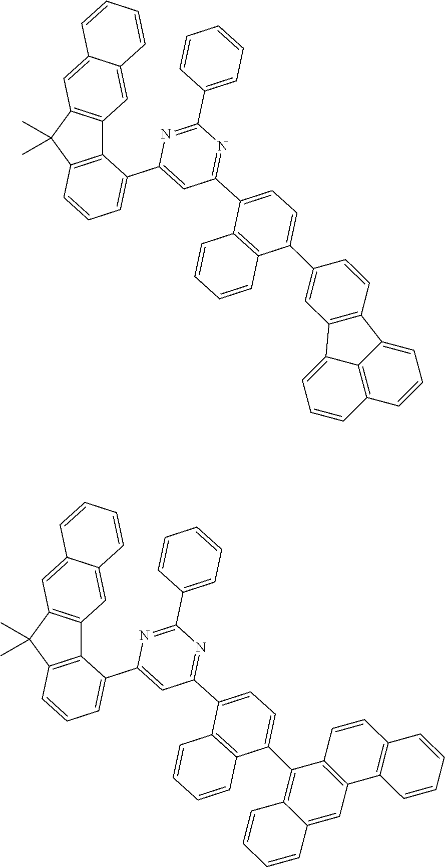 [SMILES: CC1(C)c2cc3ccccc3cc2-c2c(-c3cc(-c4ccc(-c5ccc6c(c5)-c5cccc7cccc-6c57)c5ccccc45)nc(-c4ccccc4)n3)cccc21 ; CC1(C)c2cc3ccccc3cc2-c2c(-c3cc(-c4ccc(-c5c6ccccc6cc6c5ccc5ccccc56)c5ccccc45)nc(-c4ccccc4)n3)cccc21]

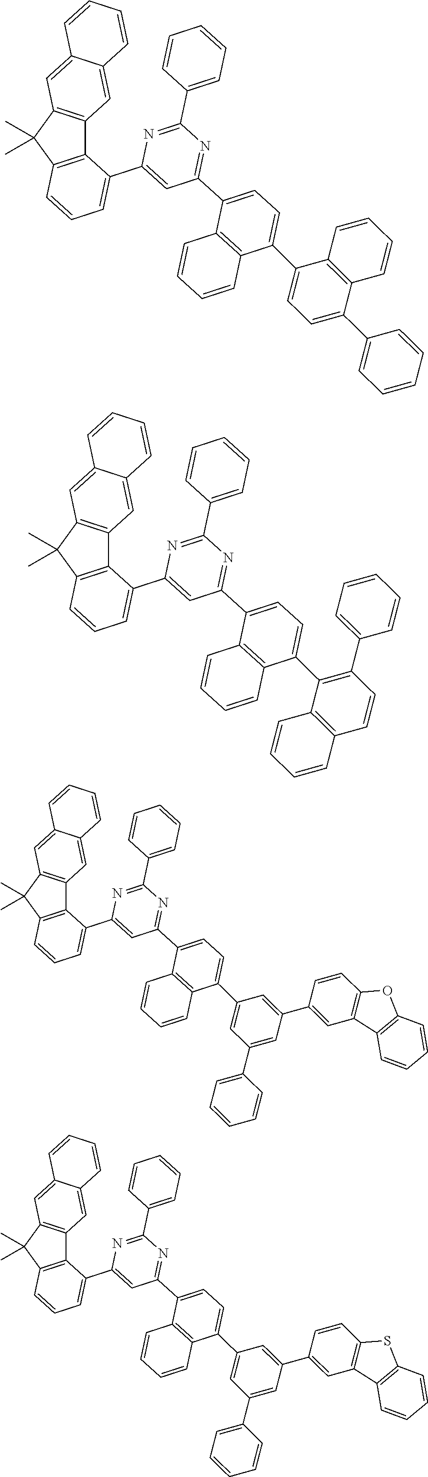 [SMILES: CC1(C)c2cc3ccccc3cc2-c2c(-c3cc(-c4ccc(-c5ccc(-c6ccccc6)c6ccccc56)c5ccccc45)nc(-c4ccccc4)n3)cccc21 ; CC1(C)c2cc3ccccc3cc2-c2c(-c3cc(-c4ccc(-c5c(-c6ccccc6)ccc6ccccc56)c5ccccc45)nc(-c4ccccc4)n3)cccc21 ; CC1(C)c2cc3ccccc3cc2-c2c(-c3cc(-c4ccc(-c5cc(-c6ccccc6)cc(-c6ccc7oc8ccccc8c7c6)c5)c5ccccc45)nc(-c4ccccc4)n3)cccc21 ; CC1(C)c2cc3ccccc3cc2-c2c(-c3cc(-c4ccc(-c5cc(-c6ccccc6)cc(-c6ccc7sc8ccccc8c7c6)c5)c5ccccc45)nc(-c4ccccc4)n3)cccc21]

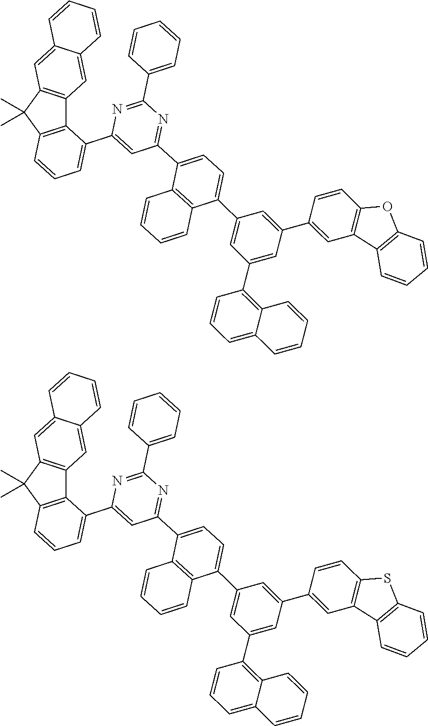 [SMILES: CC1(C)c2cc3ccccc3cc2-c2c(-c3cc(-c4ccc(-c5cc(-c6ccc7oc8ccccc8c7c6)cc(-c6cccc7ccccc67)c5)c5ccccc45)nc(-c4ccccc4)n3)cccc21 ; CC1(C)c2cc3ccccc3cc2-c2c(-c3cc(-c4ccc(-c5cc(-c6ccc7sc8ccccc8c7c6)cc(-c6cccc7ccccc67)c5)c5ccccc45)nc(-c4ccccc4)n3)cccc21]

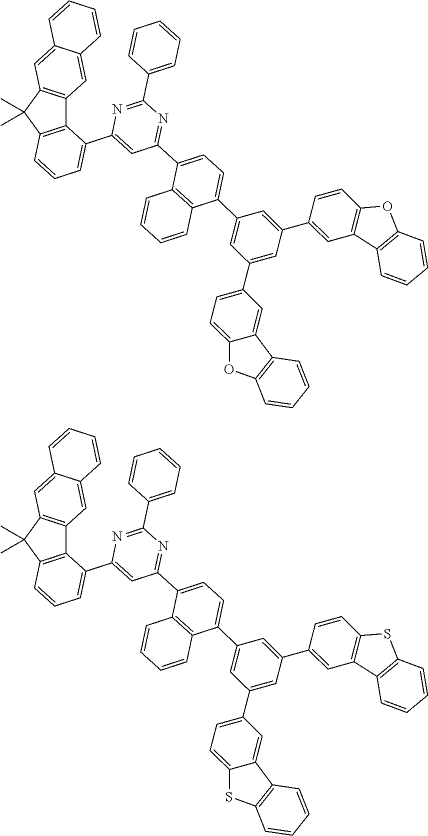 [SMILES: CC1(C)c2cc3ccccc3cc2-c2c(-c3cc(-c4ccc(-c5cc(-c6ccc7oc8ccccc8c7c6)cc(-c6ccc7oc8ccccc8c7c6)c5)c5ccccc45)nc(-c4ccccc4)n3)cccc21 ; CC1(C)c2cc3ccccc3cc2-c2c(-c3cc(-c4ccc(-c5cc(-c6ccc7sc8ccccc8c7c6)cc(-c6ccc7sc8ccccc8c7c6)c5)c5ccccc45)nc(-c4ccccc4)n3)cccc21]

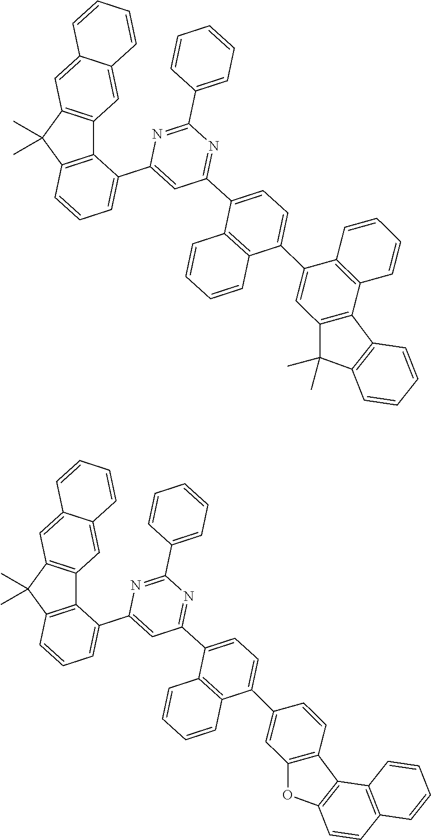 [SMILES: CC1(C)c2cc3ccccc3cc2-c2c(-c3cc(-c4ccc(-c5cc6c(c7ccccc57)-c5ccccc5C6(C)C)c5ccccc45)nc(-c4ccccc4)n3)cccc21 ; CC1(C)c2cc3ccccc3cc2-c2c(-c3cc(-c4ccc(-c5ccc6c(c5)oc5ccc7ccccc7c56)c5ccccc45)nc(-c4ccccc4)n3)cccc21]

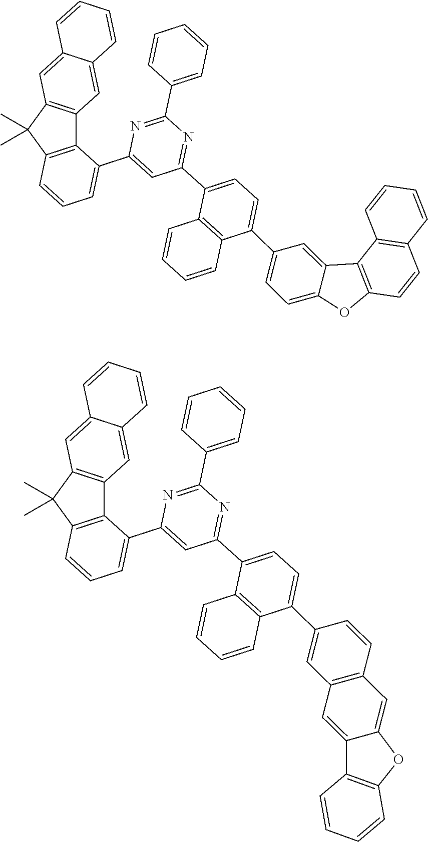 [SMILES: CC1(C)c2cc3ccccc3cc2-c2c(-c3cc(-c4ccc(-c5ccc6oc7ccc8ccccc8c7c6c5)c5ccccc45)nc(-c4ccccc4)n3)cccc21 ; CC1(C)c2cc3ccccc3cc2-c2c(-c3cc(-c4ccc(-c5ccc6cc7oc8ccccc8c7cc6c5)c5ccccc45)nc(-c4ccccc4)n3)cccc21]

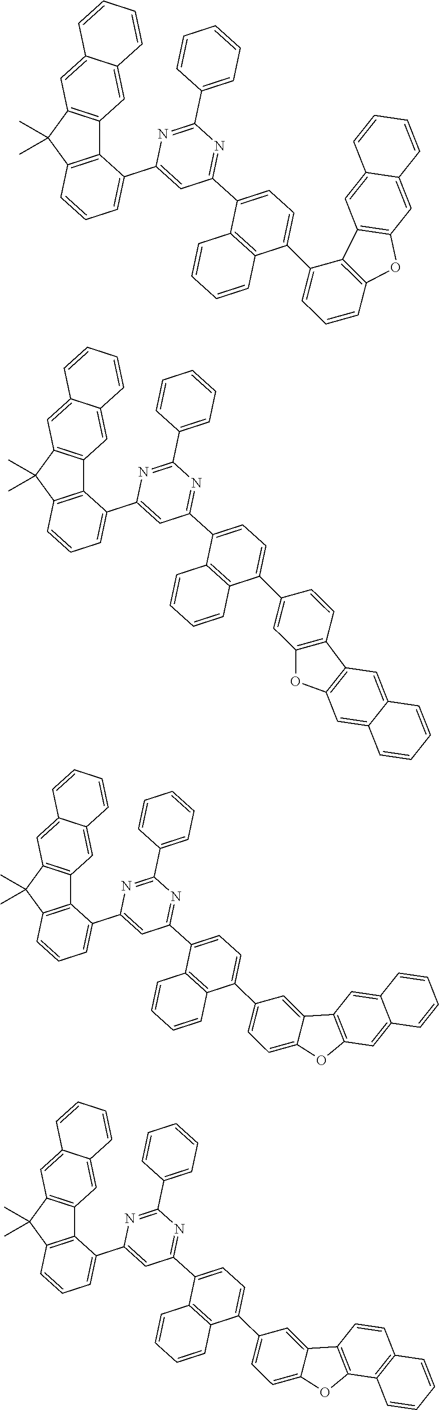 [SMILES: CC1(C)c2cc3ccccc3cc2-c2c(-c3cc(-c4ccc(-c5cccc6oc7cc8ccccc8cc7c56)c5ccccc45)nc(-c4ccccc4)n3)cccc21 ; CC1(C)c2cc3ccccc3cc2-c2c(-c3cc(-c4ccc(-c5ccc6c(c5)oc5cc7ccccc7cc56)c5ccccc45)nc(-c4ccccc4)n3)cccc21 ; CC1(C)c2cc3ccccc3cc2-c2c(-c3cc(-c4ccc(-c5ccc6oc7cc8ccccc8cc7c6c5)c5ccccc45)nc(-c4ccccc4)n3)cccc21 ; CC1(C)c2cc3ccccc3cc2-c2c(-c3cc(-c4ccc(-c5ccc6oc7c8ccccc8ccc7c6c5)c5ccccc45)nc(-c4ccccc4)n3)cccc21]

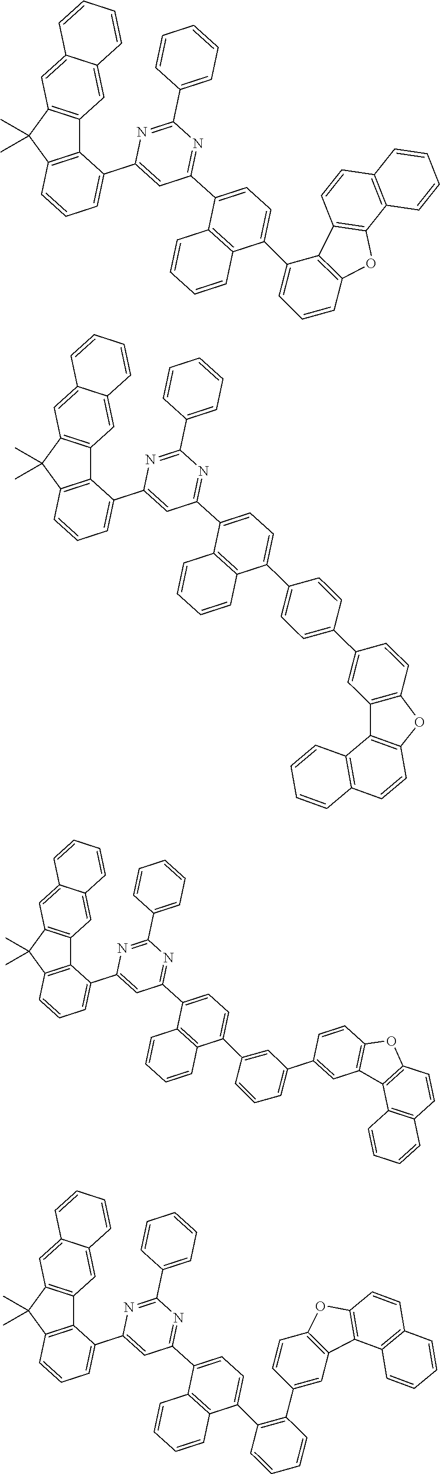 [SMILES: CC1(C)c2cc3ccccc3cc2-c2c(-c3cc(-c4ccc(-c5cccc6oc7c8ccccc8ccc7c56)c5ccccc45)nc(-c4ccccc4)n3)cccc21 ; CC1(C)c2cc3ccccc3cc2-c2c(-c3cc(-c4ccc(-c5ccc(-c6ccc7oc8ccc9ccccc9c8c7c6)cc5)c5ccccc45)nc(-c4ccccc4)n3)cccc21 ; CC1(C)c2cc3ccccc3cc2-c2c(-c3cc(-c4ccc(-c5cccc(-c6ccc7oc8ccc9ccccc9c8c7c6)c5)c5ccccc45)nc(-c4ccccc4)n3)cccc21 ; CC1(C)c2cc3ccccc3cc2-c2c(-c3cc(-c4ccc(-c5ccccc5-c5ccc6oc7ccc8ccccc8c7c6c5)c5ccccc45)nc(-c4ccccc4)n3)cccc21]

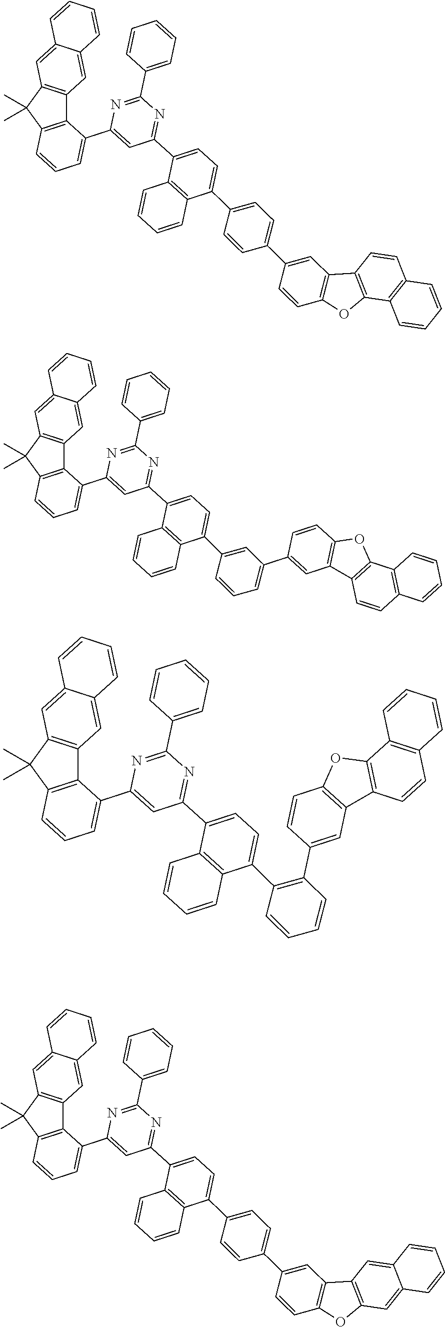 [SMILES: CC1(C)c2cc3ccccc3cc2-c2c(-c3cc(-c4ccc(-c5ccc(-c6ccc7oc8c9ccccc9ccc8c7c6)cc5)c5ccccc45)nc(-c4ccccc4)n3)cccc21 ; CC1(C)c2cc3ccccc3cc2-c2c(-c3cc(-c4ccc(-c5cccc(-c6ccc7oc8c9ccccc9ccc8c7c6)c5)c5ccccc45)nc(-c4ccccc4)n3)cccc21 ; CC1(C)c2cc3ccccc3cc2-c2c(-c3cc(-c4ccc(-c5ccccc5-c5ccc6oc7c8ccccc8ccc7c6c5)c5ccccc45)nc(-c4ccccc4)n3)cccc21 ; CC1(C)c2cc3ccccc3cc2-c2c(-c3cc(-c4ccc(-c5ccc(-c6ccc7oc8cc9ccccc9cc8c7c6)cc5)c5ccccc45)nc(-c4ccccc4)n3)cccc21]

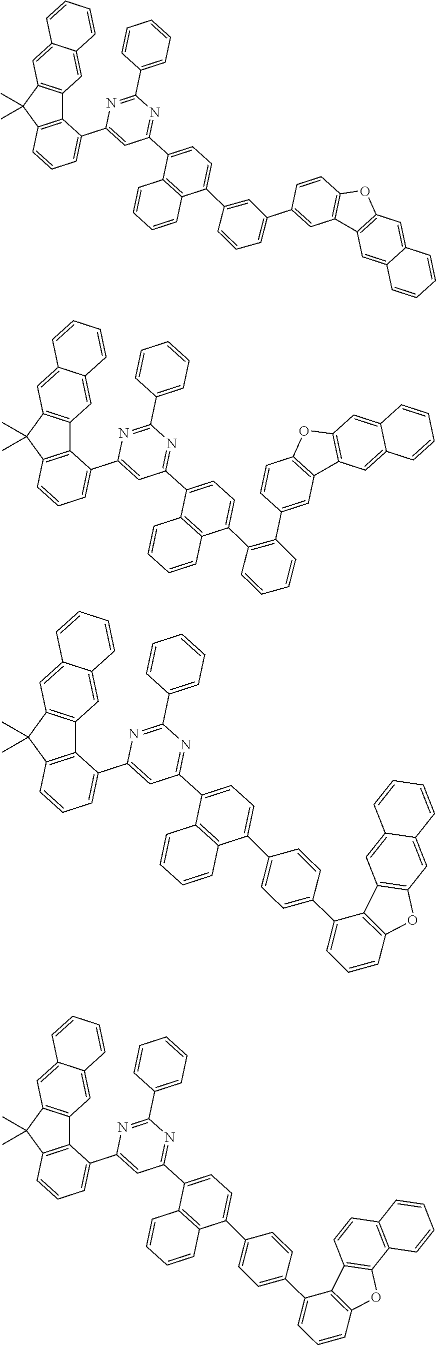 [SMILES: CC1(C)c2cc3ccccc3cc2-c2c(-c3cc(-c4ccc(-c5cccc(-c6ccc7oc8cc9ccccc9cc8c7c6)c5)c5ccccc45)nc(-c4ccccc4)n3)cccc21 ; CC1(C)c2cc3ccccc3cc2-c2c(-c3cc(-c4ccc(-c5ccccc5-c5ccc6oc7cc8ccccc8cc7c6c5)c5ccccc45)nc(-c4ccccc4)n3)cccc21 ; CC1(C)c2cc3ccccc3cc2-c2c(-c3cc(-c4ccc(-c5ccc(-c6cccc7oc8cc9ccccc9cc8c67)cc5)c5ccccc45)nc(-c4ccccc4)n3)cccc21 ; CC1(C)c2cc3ccccc3cc2-c2c(-c3cc(-c4ccc(-c5ccc(-c6cccc7oc8c9ccccc9ccc8c67)cc5)c5ccccc45)nc(-c4ccccc4)n3)cccc21]

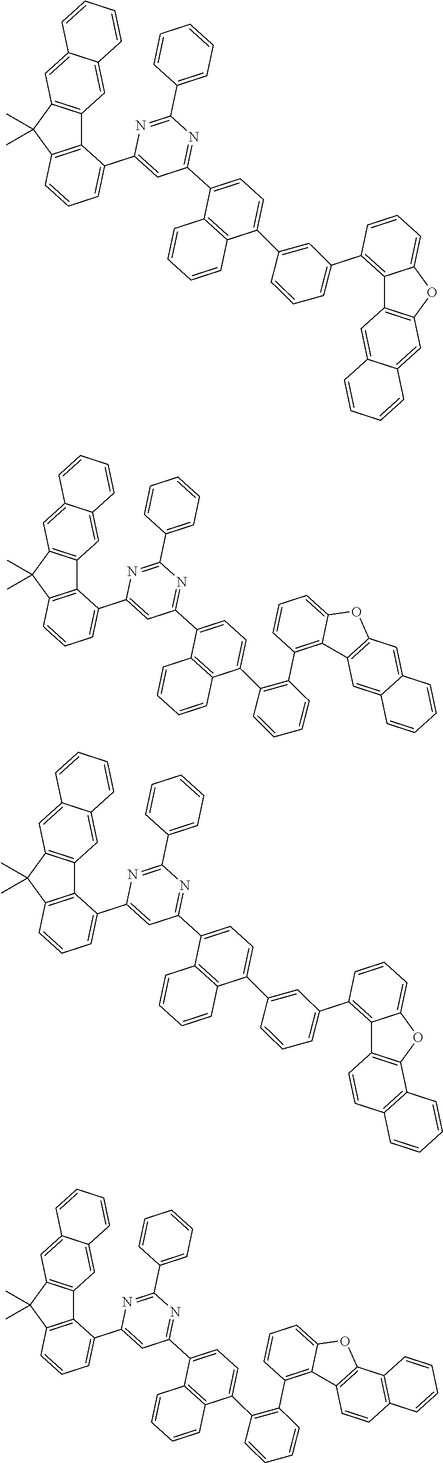 [SMILES: CC1(C)c2cc3ccccc3cc2-c2c(-c3cc(-c4ccc(-c5cccc(-c6cccc7oc8cc9ccccc9cc8c67)c5)c5ccccc45)nc(-c4ccccc4)n3)cccc21 ; CC1(C)c2cc3ccccc3cc2-c2c(-c3cc(-c4ccc(-c5ccccc5-c5cccc6oc7cc8ccccc8cc7c56)c5ccccc45)nc(-c4ccccc4)n3)cccc21 ; CC1(C)c2cc3ccccc3cc2-c2c(-c3cc(-c4ccc(-c5cccc(-c6cccc7oc8c9ccccc9ccc8c67)c5)c5ccccc45)nc(-c4ccccc4)n3)cccc21 ; CC1(C)c2cc3ccccc3cc2-c2c(-c3cc(-c4ccc(-c5ccccc5-c5cccc6oc7c8ccccc8ccc7c56)c5ccccc45)nc(-c4ccccc4)n3)cccc21]

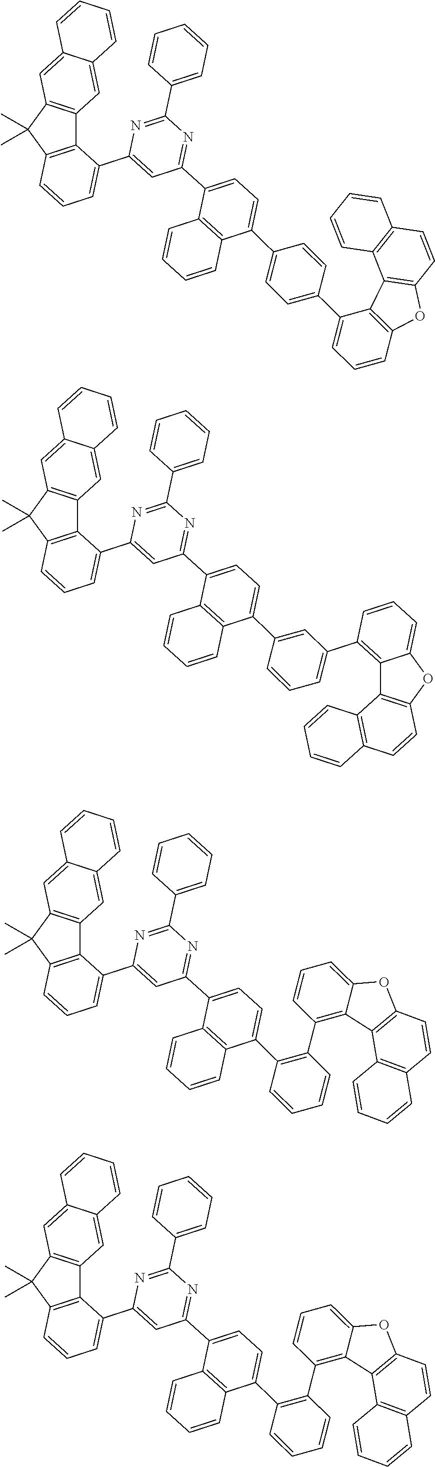 [SMILES: CC1(C)c2cc3ccccc3cc2-c2c(-c3cc(-c4ccc(-c5ccc(-c6cccc7oc8ccc9ccccc9c8c67)cc5)c5ccccc45)nc(-c4ccccc4)n3)cccc21 ; CC1(C)c2cc3ccccc3cc2-c2c(-c3cc(-c4ccc(-c5cccc(-c6cccc7oc8ccc9ccccc9c8c67)c5)c5ccccc45)nc(-c4ccccc4)n3)cccc21 ; CC1(C)c2cc3ccccc3cc2-c2c(-c3cc(-c4ccc(-c5ccccc5-c5cccc6oc7ccc8ccccc8c7c56)c5ccccc45)nc(-c4ccccc4)n3)cccc21 ; CC1(C)c2cc3ccccc3cc2-c2c(-c3cc(-c4ccc(-c5ccccc5-c5cccc6oc7ccc8ccccc8c7c56)c5ccccc45)nc(-c4ccccc4)n3)cccc21]

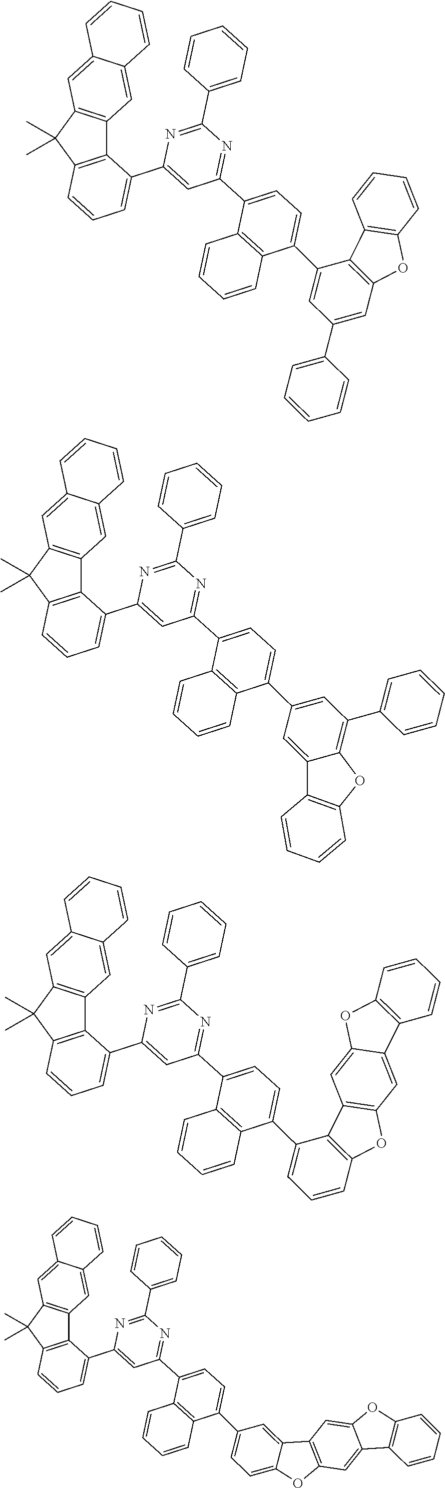 [SMILES: CC1(C)c2cc3ccccc3cc2-c2c(-c3cc(-c4ccc(-c5cc(-c6ccccc6)cc6oc7ccccc7c56)c5ccccc45)nc(-c4ccccc4)n3)cccc21 ; CC1(C)c2cc3ccccc3cc2-c2c(-c3cc(-c4ccc(-c5cc(-c6ccccc6)c6oc7ccccc7c6c5)c5ccccc45)nc(-c4ccccc4)n3)cccc21 ; CC1(C)c2cc3ccccc3cc2-c2c(-c3cc(-c4ccc(-c5cccc6oc7cc8c(cc7c56)oc5ccccc58)c5ccccc45)nc(-c4ccccc4)n3)cccc21 ; CC1(C)c2cc3ccccc3cc2-c2c(-c3cc(-c4ccc(-c5ccc6oc7cc8c(cc7c6c5)oc5ccccc58)c5ccccc45)nc(-c4ccccc4)n3)cccc21]

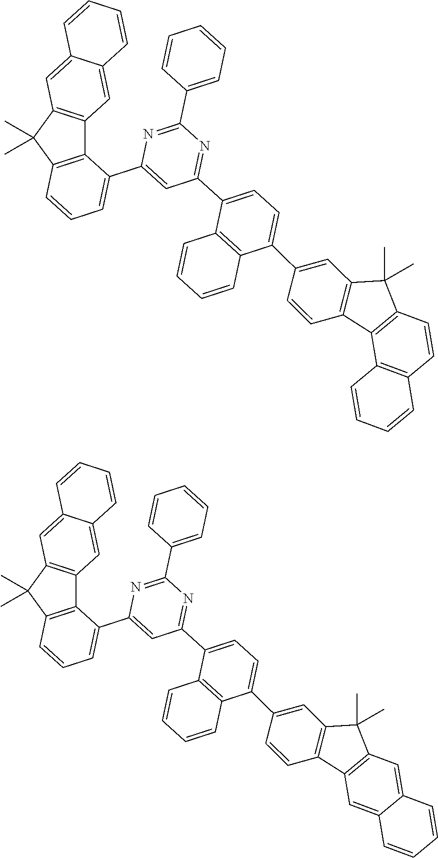 [SMILES: CC1(C)c2cc3ccccc3cc2-c2c(-c3cc(-c4ccc(-c5ccc6c(c5)C(C)(C)c5ccc7ccccc7c5-6)c5ccccc45)nc(-c4ccccc4)n3)cccc21 ; CC1(C)c2cc(-c3ccc(-c4cc(-c5cccc6c5-c5cc7ccccc7cc5C6(C)C)nc(-c5ccccc5)n4)c4ccccc34)ccc2-c2cc3ccccc3cc21]

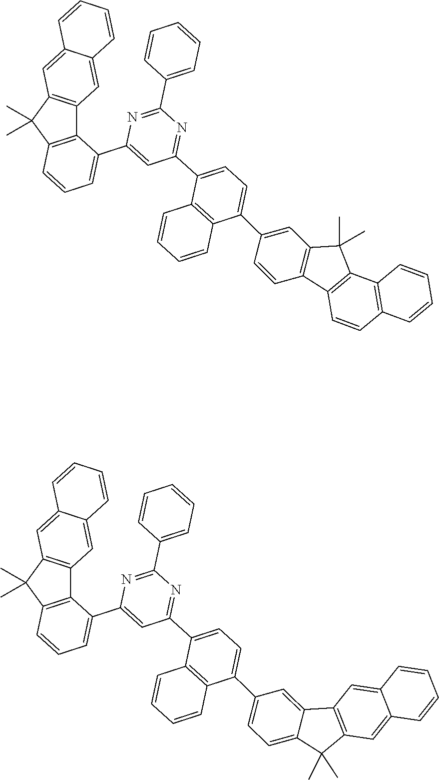 [SMILES: CC1(C)c2cc3ccccc3cc2-c2c(-c3cc(-c4ccc(-c5ccc6c(c5)C(C)(C)c5c-6ccc6ccccc56)c5ccccc45)nc(-c4ccccc4)n3)cccc21 ; CC1(C)c2ccc(-c3ccc(-c4cc(-c5cccc6c5-c5cc7ccccc7cc5C6(C)C)nc(-c5ccccc5)n4)c4ccccc34)cc2-c2cc3ccccc3cc21]

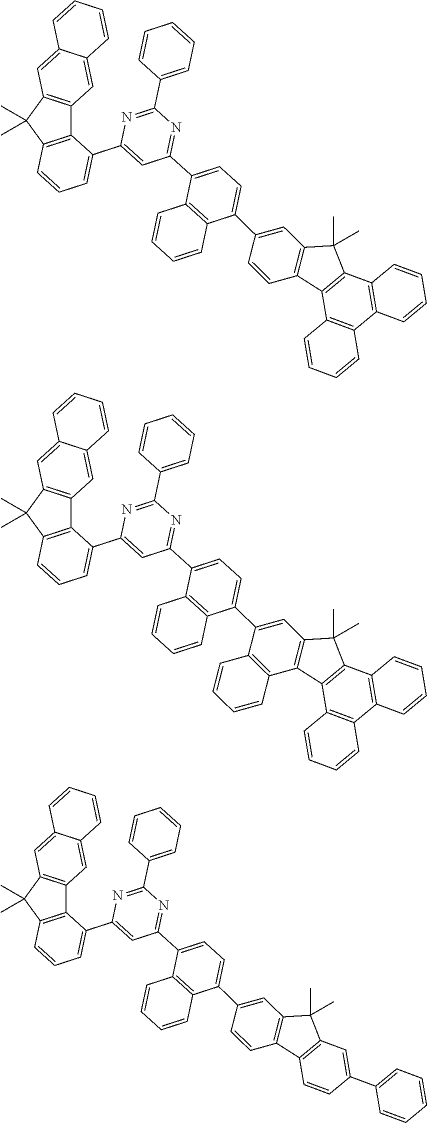 [SMILES: CC1(C)c2cc3ccccc3cc2-c2c(-c3cc(-c4ccc(-c5ccc6c(c5)C(C)(C)c5c-6c6ccccc6c6ccccc56)c5ccccc45)nc(-c4ccccc4)n3)cccc21 ; CC1(C)c2cc3ccccc3cc2-c2c(-c3cc(-c4ccc(-c5cc6c(c7ccccc57)-c5c(c7ccccc7c7ccccc57)C6(C)C)c5ccccc45)nc(-c4ccccc4)n3)cccc21 ; CC1(C)c2cc(-c3ccccc3)ccc2-c2ccc(-c3ccc(-c4cc(-c5cccc6c5-c5cc7ccccc7cc5C6(C)C)nc(-c5ccccc5)n4)c4ccccc34)cc21]

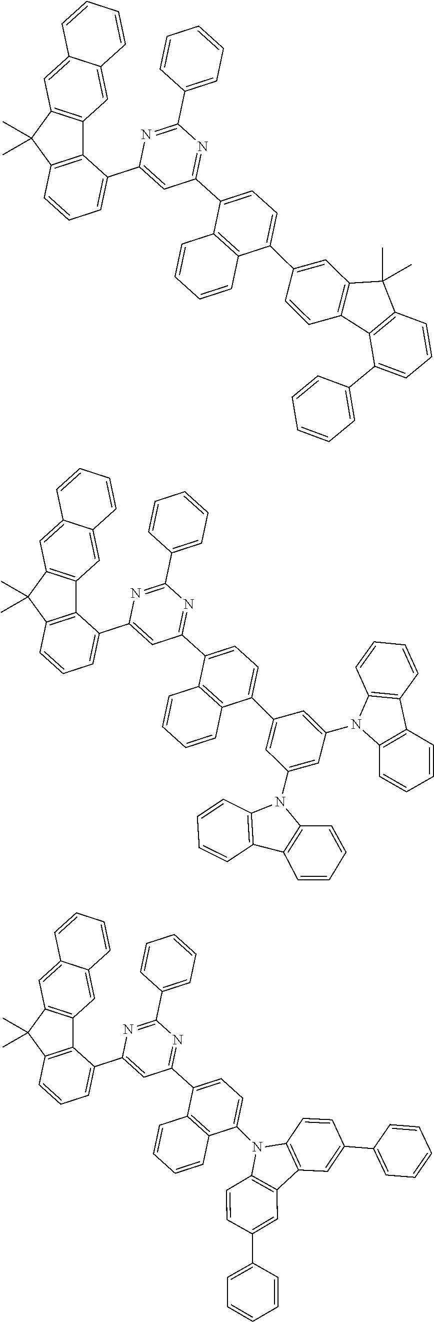 [SMILES: CC1(C)c2cc(-c3ccc(-c4cc(-c5cccc6c5-c5cc7ccccc7cc5C6(C)C)nc(-c5ccccc5)n4)c4ccccc34)ccc2-c2c(-c3ccccc3)cccc21 ; CC1(C)c2cc3ccccc3cc2-c2c(-c3cc(-c4ccc(-c5cc(-n6c7ccccc7c7ccccc76)cc(-n6c7ccccc7c7ccccc76)c5)c5ccccc45)nc(-c4ccccc4)n3)cccc21 ; CC1(C)c2cc3ccccc3cc2-c2c(-c3cc(-c4ccc(-n5c6ccc(-c7ccccc7)cc6c6cc(-c7ccccc7)ccc65)c5ccccc45)nc(-c4ccccc4)n3)cccc21]

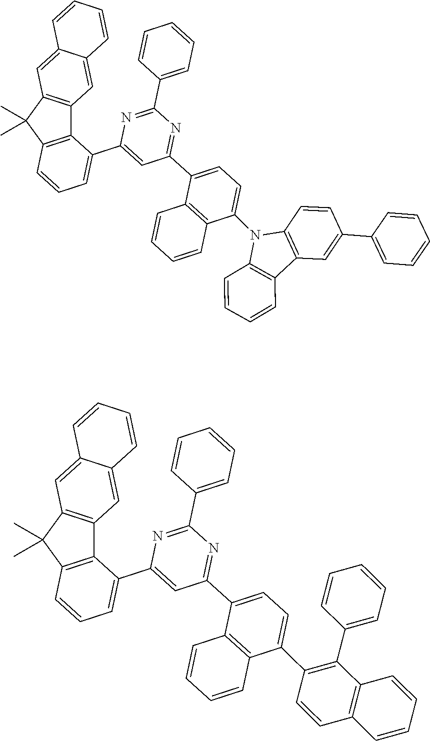 [SMILES: CC1(C)c2cc3ccccc3cc2-c2c(-c3cc(-c4ccc(-n5c6ccccc6c6cc(-c7ccccc7)ccc65)c5ccccc45)nc(-c4ccccc4)n3)cccc21 ; CC1(C)c2cc3ccccc3cc2-c2c(-c3cc(-c4ccc(-c5ccc6ccccc6c5-c5ccccc5)c5ccccc45)nc(-c4ccccc4)n3)cccc21]

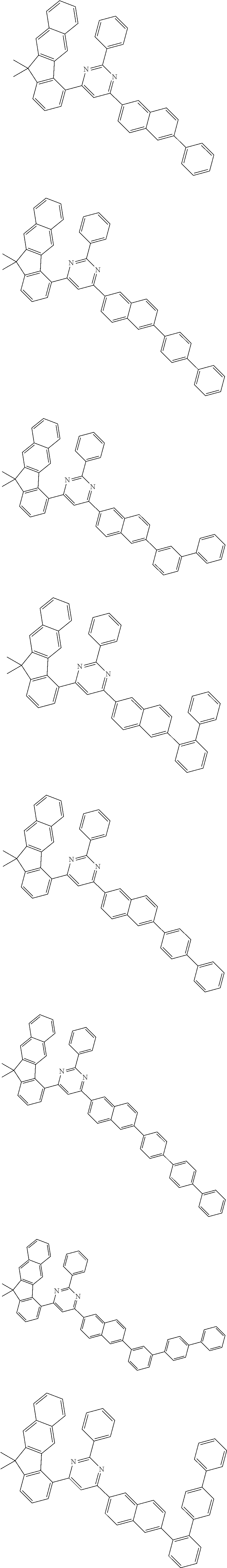

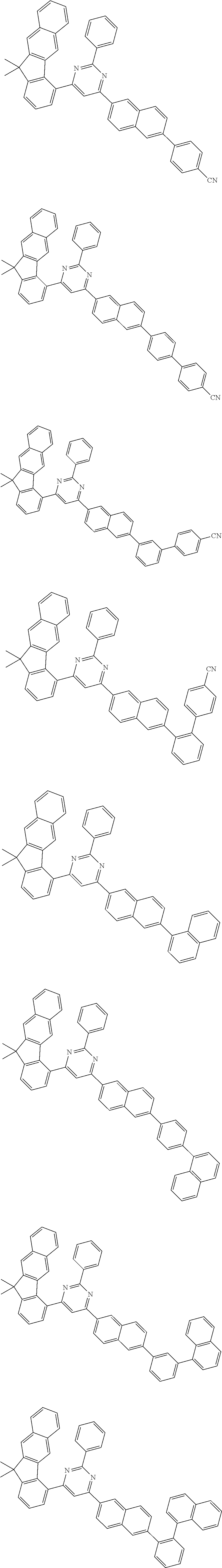

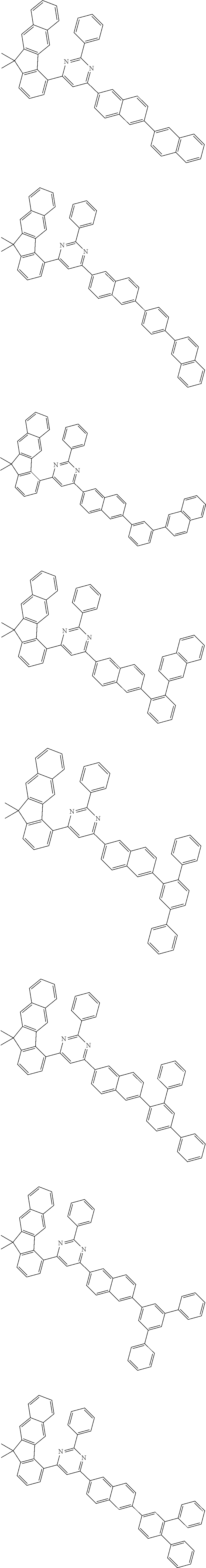

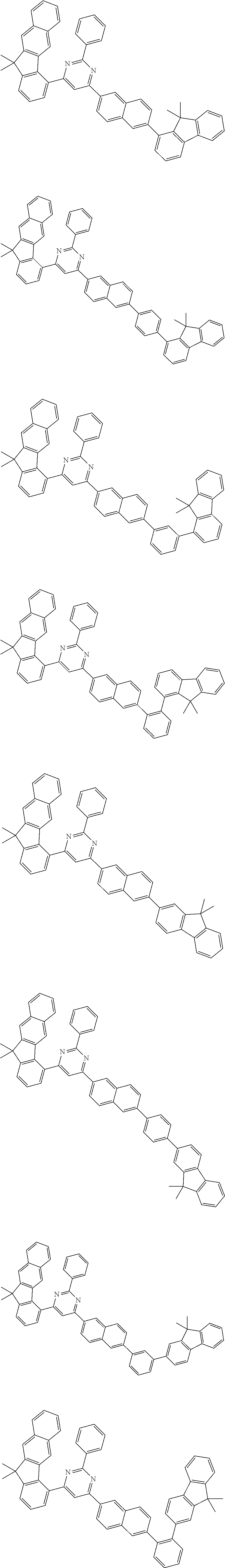

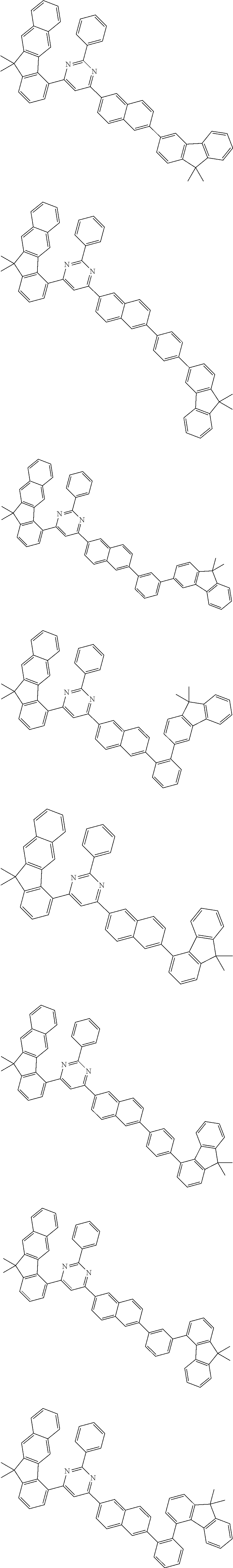

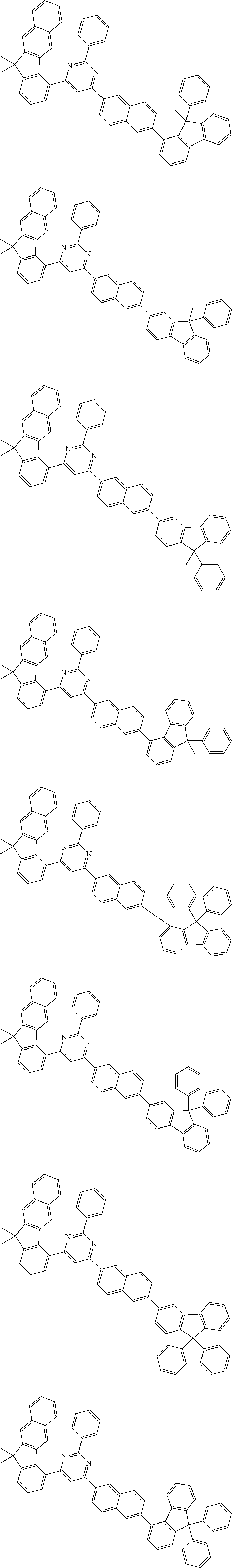

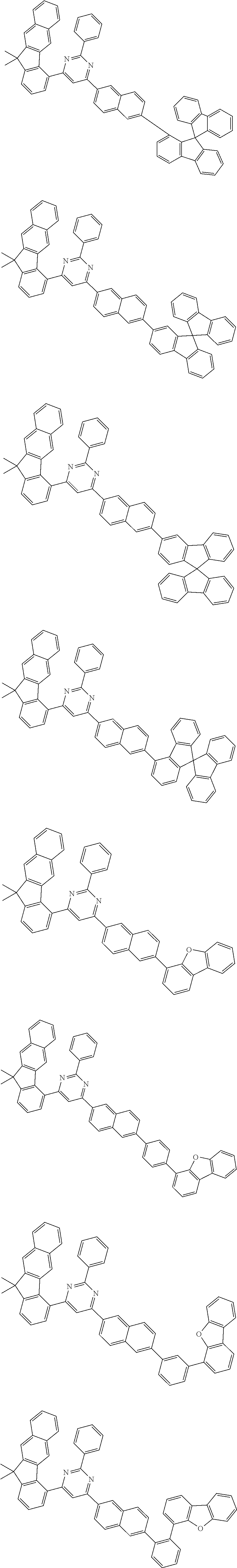

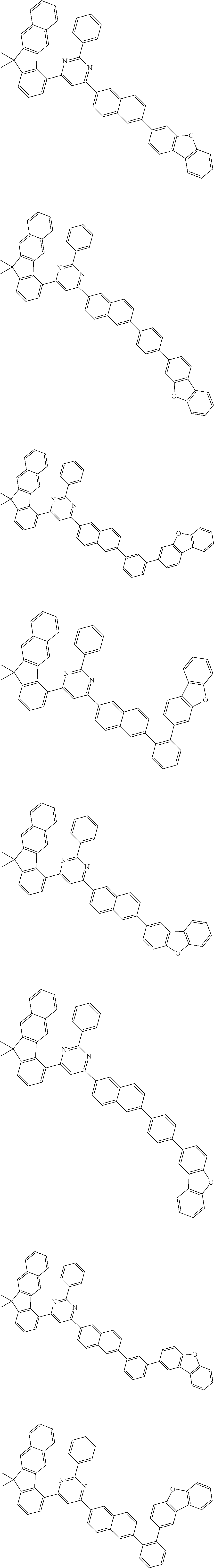

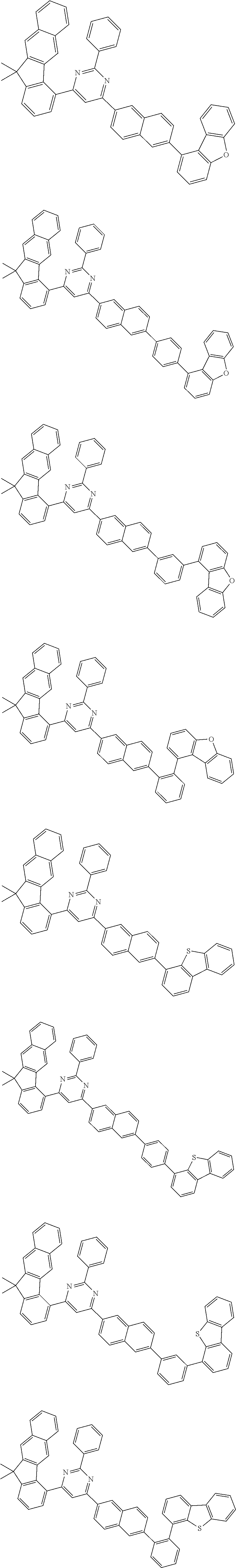

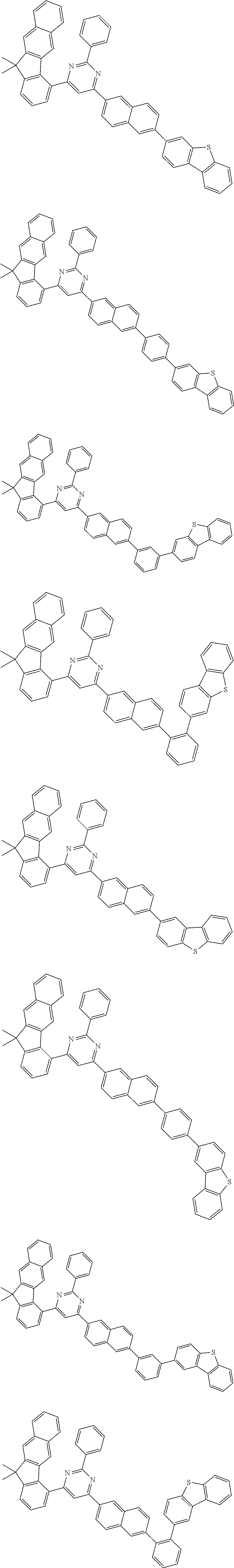

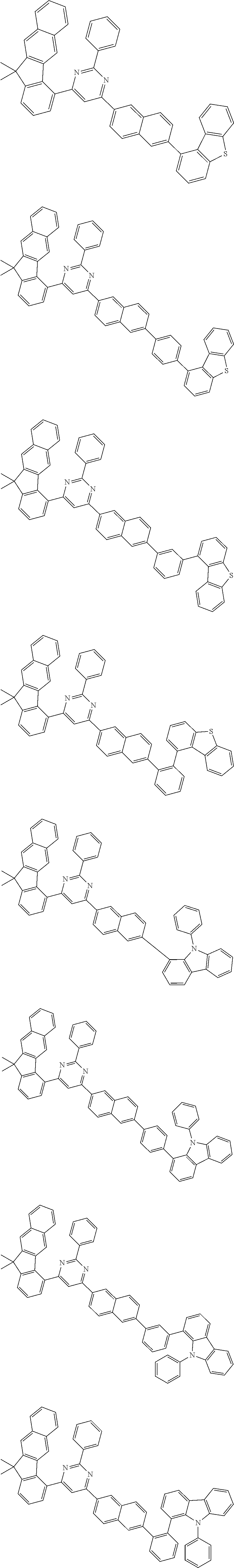

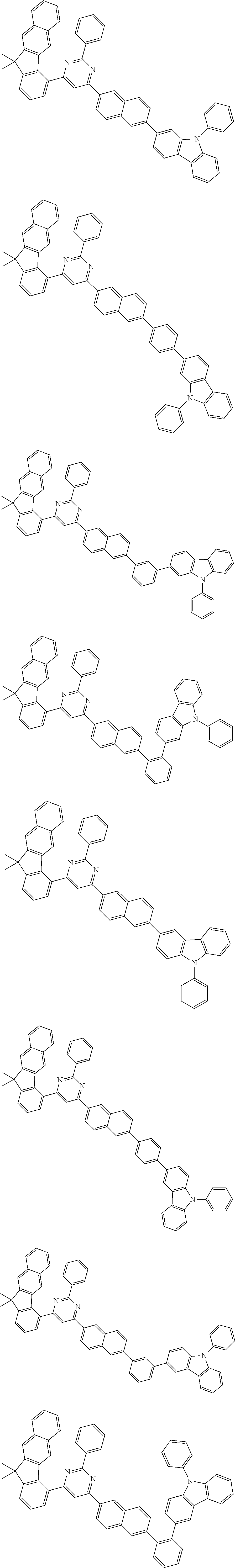

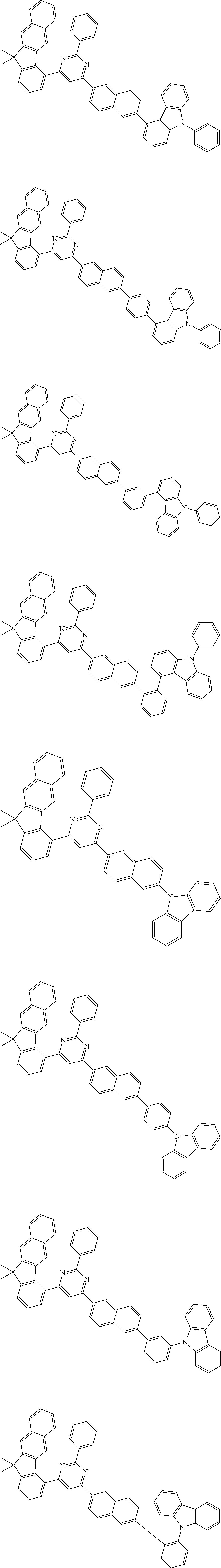

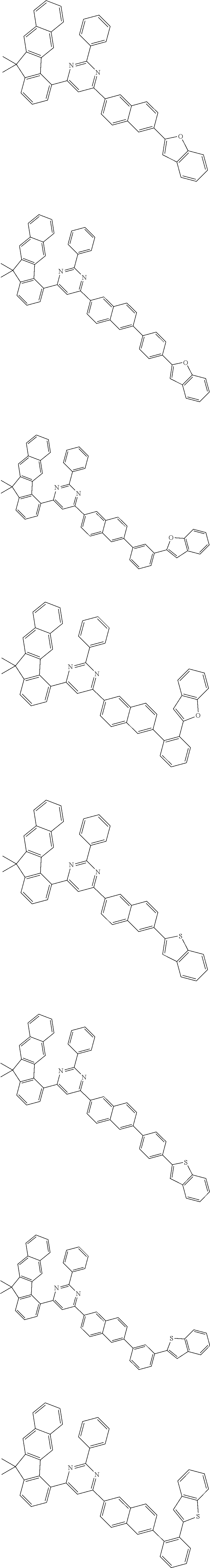

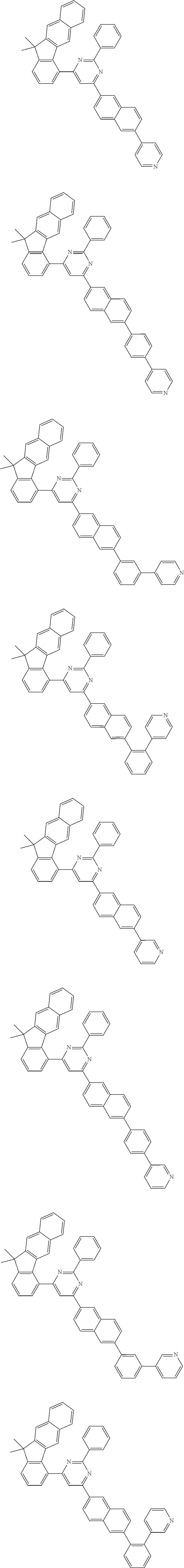

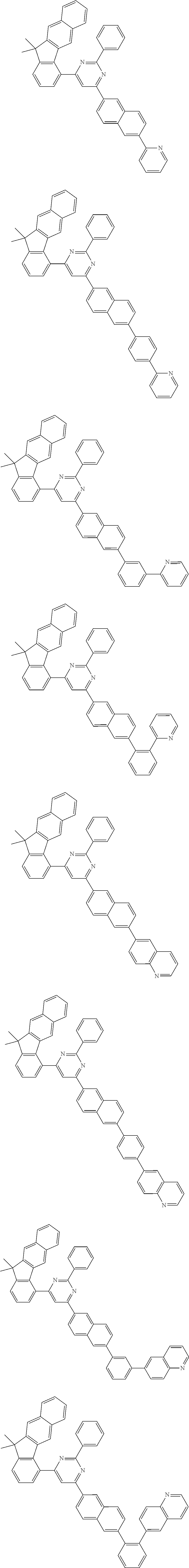

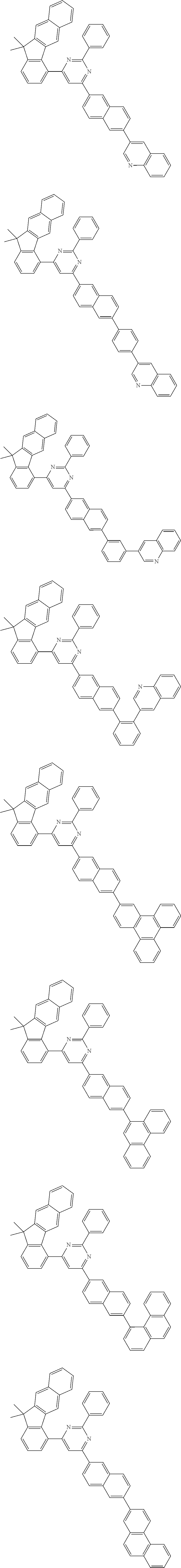

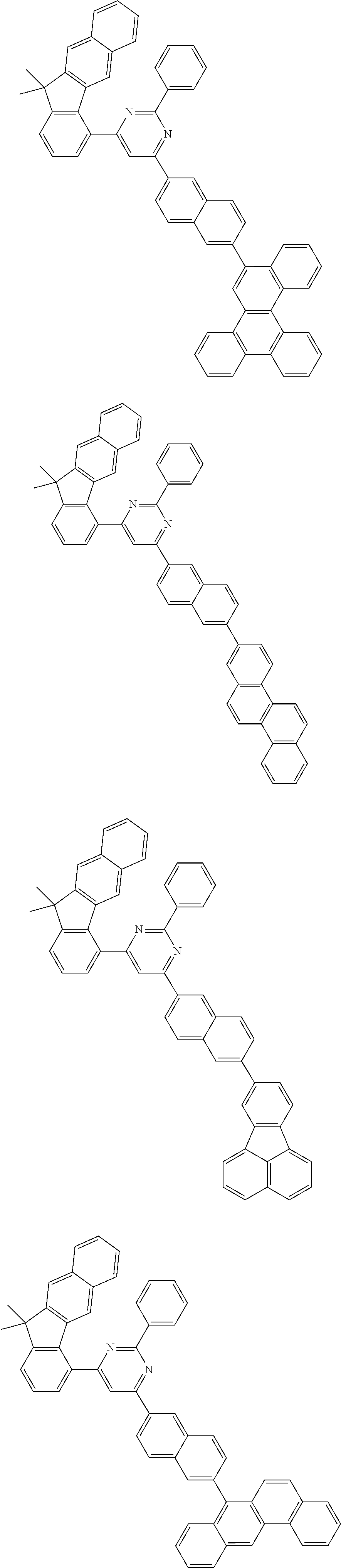 [SMILES: CC1(C)c2cc3ccccc3cc2-c2c(-c3cc(-c4ccc5cc(-c6cc7c8ccccc8c8ccccc8c7c7ccccc67)ccc5c4)nc(-c4ccccc4)n3)cccc21 ; CC1(C)c2cc3ccccc3cc2-c2c(-c3cc(-c4ccc5cc(-c6ccc7c(ccc8c9ccccc9ccc78)c6)ccc5c4)nc(-c4ccccc4)n3)cccc21 ; CC1(C)c2cc3ccccc3cc2-c2c(-c3cc(-c4ccc5cc(-c6ccc7c(c6)-c6cccc8cccc-7c68)ccc5c4)nc(-c4ccccc4)n3)cccc21 ; CC1(C)c2cc3ccccc3cc2-c2c(-c3cc(-c4ccc5cc(-c6c7ccccc7cc7c6ccc6ccccc67)ccc5c4)nc(-c4ccccc4)n3)cccc21]

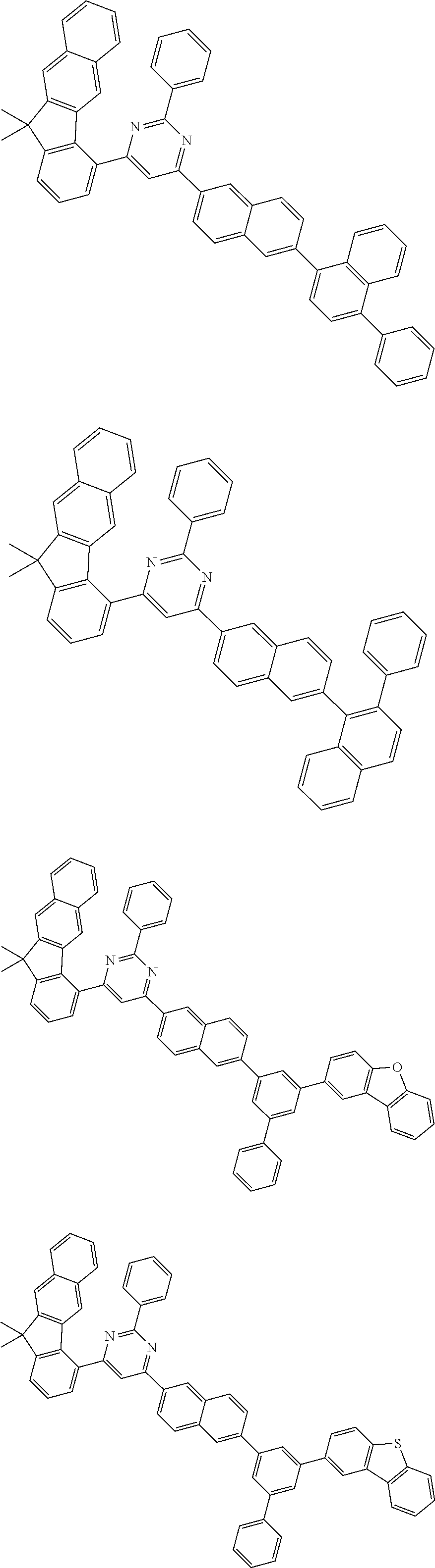 [SMILES: CC1(C)c2cc3ccccc3cc2-c2c(-c3cc(-c4ccc5cc(-c6ccc(-c7ccccc7)c7ccccc67)ccc5c4)nc(-c4ccccc4)n3)cccc21 ; CC1(C)c2cc3ccccc3cc2-c2c(-c3cc(-c4ccc5cc(-c6c(-c7ccccc7)ccc7ccccc67)ccc5c4)nc(-c4ccccc4)n3)cccc21 ; CC1(C)c2cc3ccccc3cc2-c2c(-c3cc(-c4ccc5cc(-c6cc(-c7ccccc7)cc(-c7ccc8oc9ccccc9c8c7)c6)ccc5c4)nc(-c4ccccc4)n3)cccc21 ; CC1(C)c2cc3ccccc3cc2-c2c(-c3cc(-c4ccc5cc(-c6cc(-c7ccccc7)cc(-c7ccc8sc9ccccc9c8c7)c6)ccc5c4)nc(-c4ccccc4)n3)cccc21]

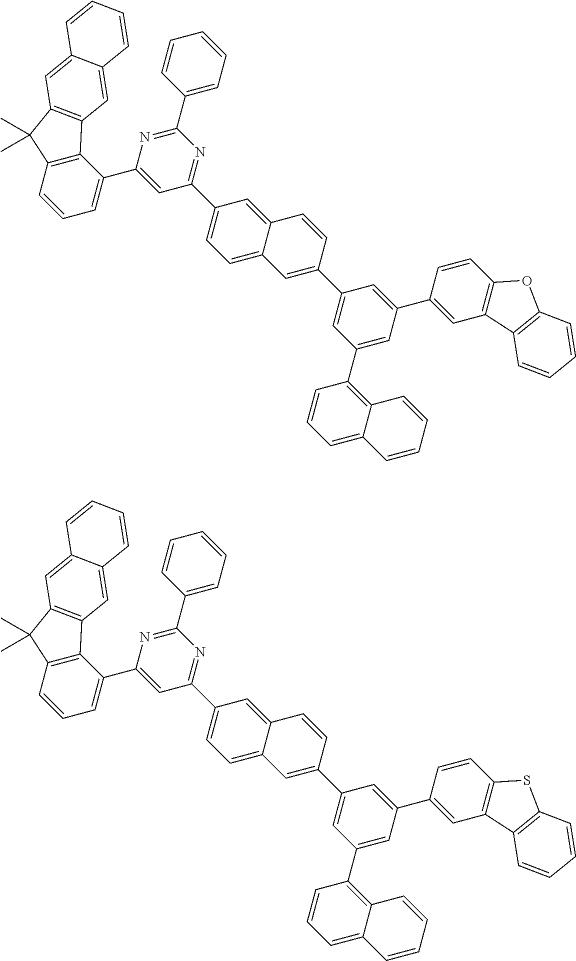 [SMILES: CC1(C)c2cc3ccccc3cc2-c2c(-c3cc(-c4ccc5cc(-c6cc(-c7ccc8oc9ccccc9c8c7)cc(-c7cccc8ccccc78)c6)ccc5c4)nc(-c4ccccc4)n3)cccc21 ; CC1(C)c2cc3ccccc3cc2-c2c(-c3cc(-c4ccc5cc(-c6cc(-c7ccc8sc9ccccc9c8c7)cc(-c7cccc8ccccc78)c6)ccc5c4)nc(-c4ccccc4)n3)cccc21]

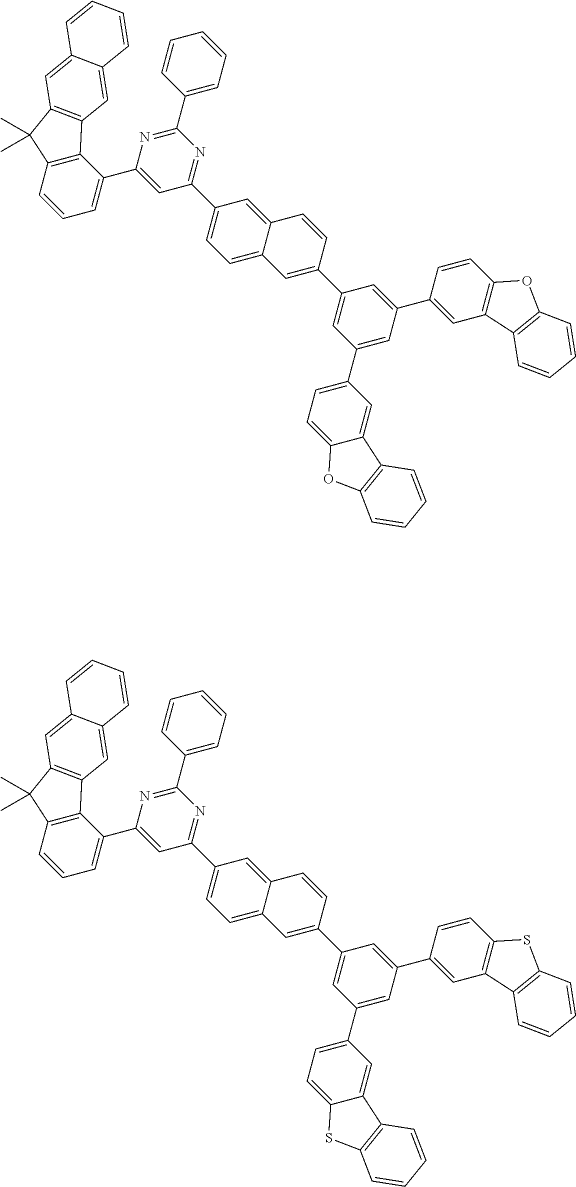 [SMILES: CC1(C)c2cc3ccccc3cc2-c2c(-c3cc(-c4ccc5cc(-c6cc(-c7ccc8oc9ccccc9c8c7)cc(-c7ccc8oc9ccccc9c8c7)c6)ccc5c4)nc(-c4ccccc4)n3)cccc21 ; CC1(C)c2cc3ccccc3cc2-c2c(-c3cc(-c4ccc5cc(-c6cc(-c7ccc8sc9ccccc9c8c7)cc(-c7ccc8sc9ccccc9c8c7)c6)ccc5c4)nc(-c4ccccc4)n3)cccc21]

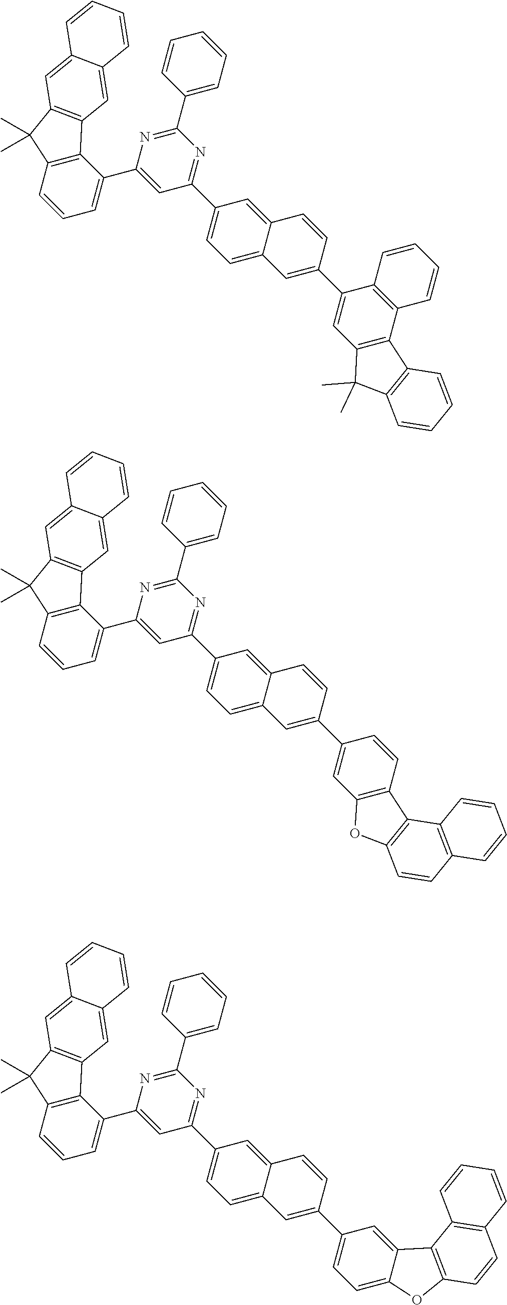 [SMILES: CC1(C)c2cc3ccccc3cc2-c2c(-c3cc(-c4ccc5cc(-c6cc7c(c8ccccc68)-c6ccccc6C7(C)C)ccc5c4)nc(-c4ccccc4)n3)cccc21 ; CC1(C)c2cc3ccccc3cc2-c2c(-c3cc(-c4ccc5cc(-c6ccc7c(c6)oc6ccc8ccccc8c67)ccc5c4)nc(-c4ccccc4)n3)cccc21 ; CC1(C)c2cc3ccccc3cc2-c2c(-c3cc(-c4ccc5cc(-c6ccc7oc8ccc9ccccc9c8c7c6)ccc5c4)nc(-c4ccccc4)n3)cccc21]

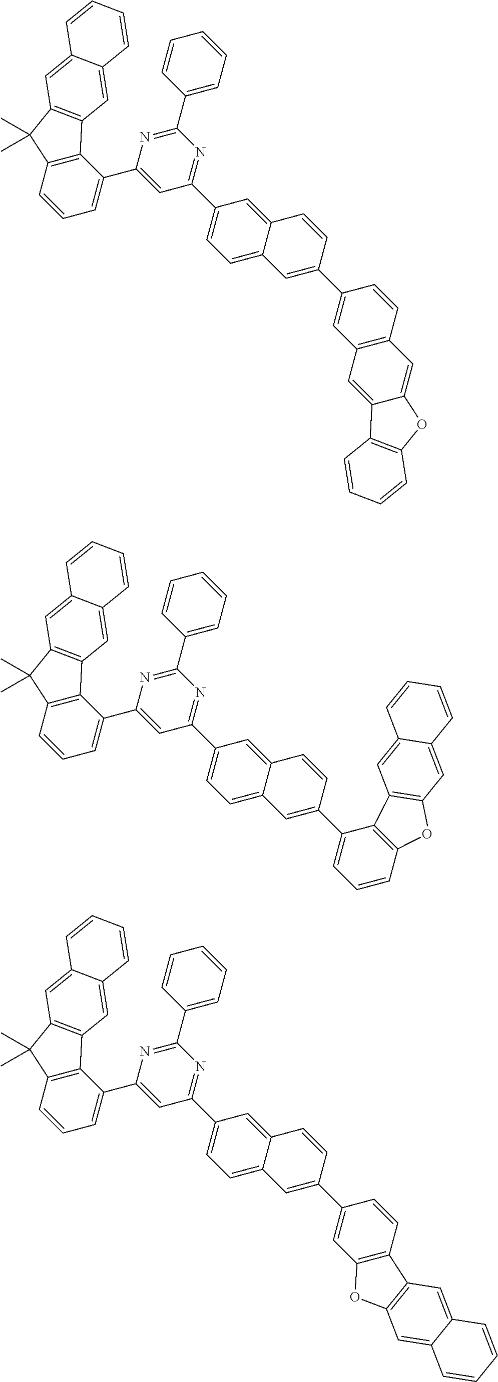 [SMILES: CC1(C)c2cc3ccccc3cc2-c2c(-c3cc(-c4ccc5cc(-c6ccc7cc8oc9ccccc9c8cc7c6)ccc5c4)nc(-c4ccccc4)n3)cccc21 ; CC1(C)c2cc3ccccc3cc2-c2c(-c3cc(-c4ccc5cc(-c6cccc7oc8cc9ccccc9cc8c67)ccc5c4)nc(-c4ccccc4)n3)cccc21 ; CC1(C)c2cc3ccccc3cc2-c2c(-c3cc(-c4ccc5cc(-c6ccc7c(c6)oc6cc8ccccc8cc67)ccc5c4)nc(-c4ccccc4)n3)cccc21]

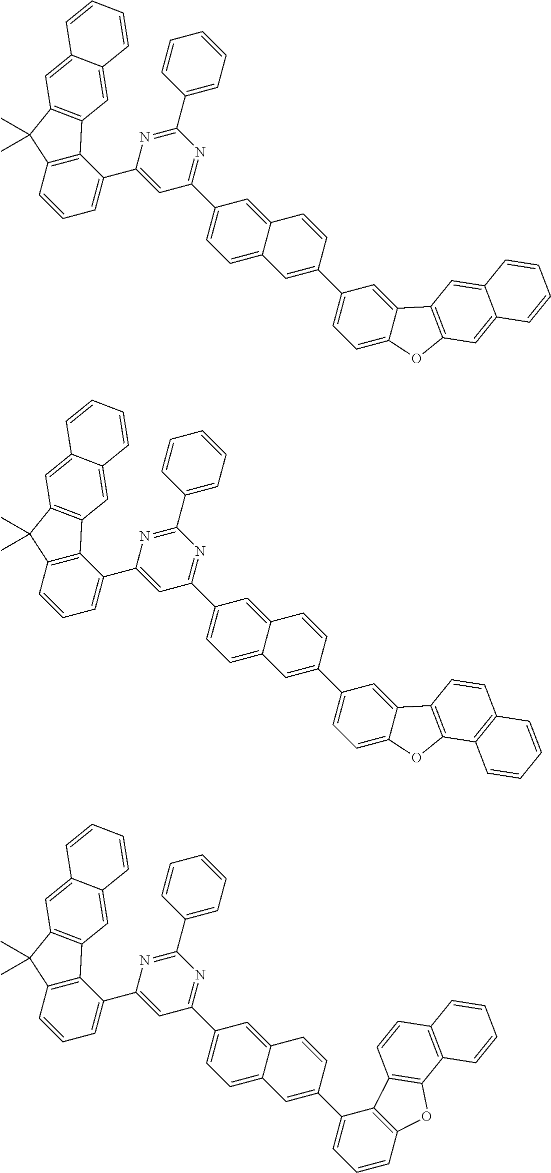 [SMILES: CC1(C)c2cc3ccccc3cc2-c2c(-c3cc(-c4ccc5cc(-c6ccc7oc8cc9ccccc9cc8c7c6)ccc5c4)nc(-c4ccccc4)n3)cccc21 ; CC1(C)c2cc3ccccc3cc2-c2c(-c3cc(-c4ccc5cc(-c6ccc7oc8c9ccccc9ccc8c7c6)ccc5c4)nc(-c4ccccc4)n3)cccc21 ; CC1(C)c2cc3ccccc3cc2-c2c(-c3cc(-c4ccc5cc(-c6cccc7oc8c9ccccc9ccc8c67)ccc5c4)nc(-c4ccccc4)n3)cccc21]

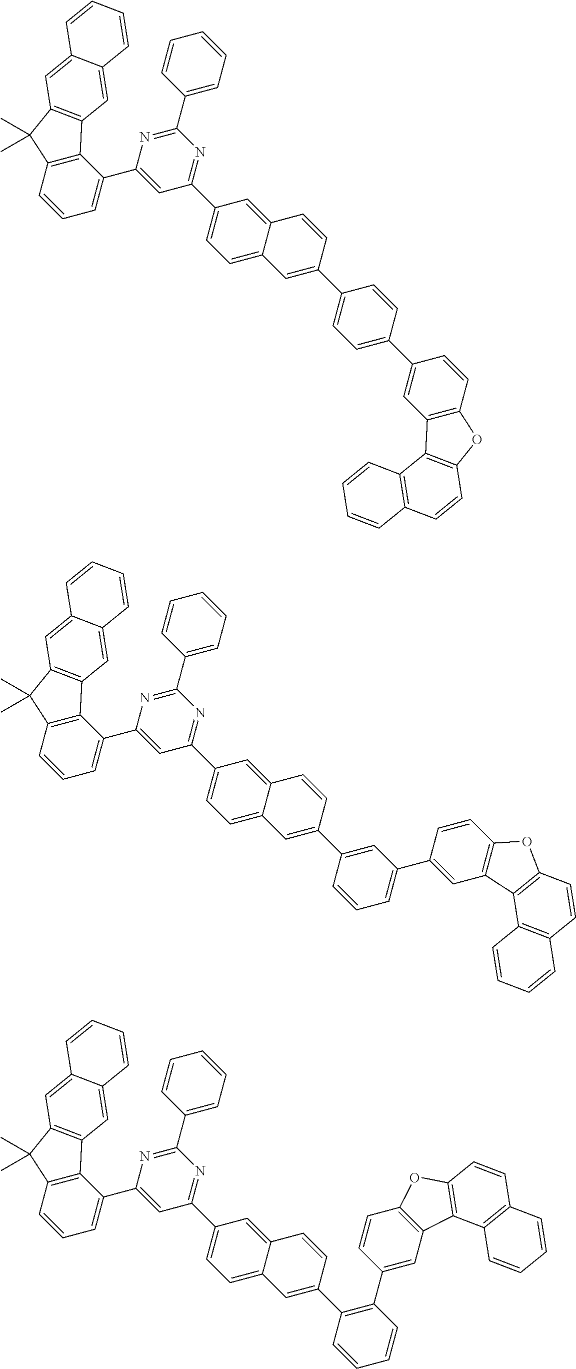 [SMILES: CC1(C)c2cc3ccccc3cc2-c2c(-c3cc(-c4ccc5cc(-c6ccc(-c7ccc8oc9ccc%10ccccc%10c9c8c7)cc6)ccc5c4)nc(-c4ccccc4)n3)cccc21 ; CC1(C)c2cc3ccccc3cc2-c2c(-c3cc(-c4ccc5cc(-c6cccc(-c7ccc8oc9ccc%10ccccc%10c9c8c7)c6)ccc5c4)nc(-c4ccccc4)n3)cccc21 ; CC1(C)c2cc3ccccc3cc2-c2c(-c3cc(-c4ccc5cc(-c6ccccc6-c6ccc7oc8ccc9ccccc9c8c7c6)ccc5c4)nc(-c4ccccc4)n3)cccc21]

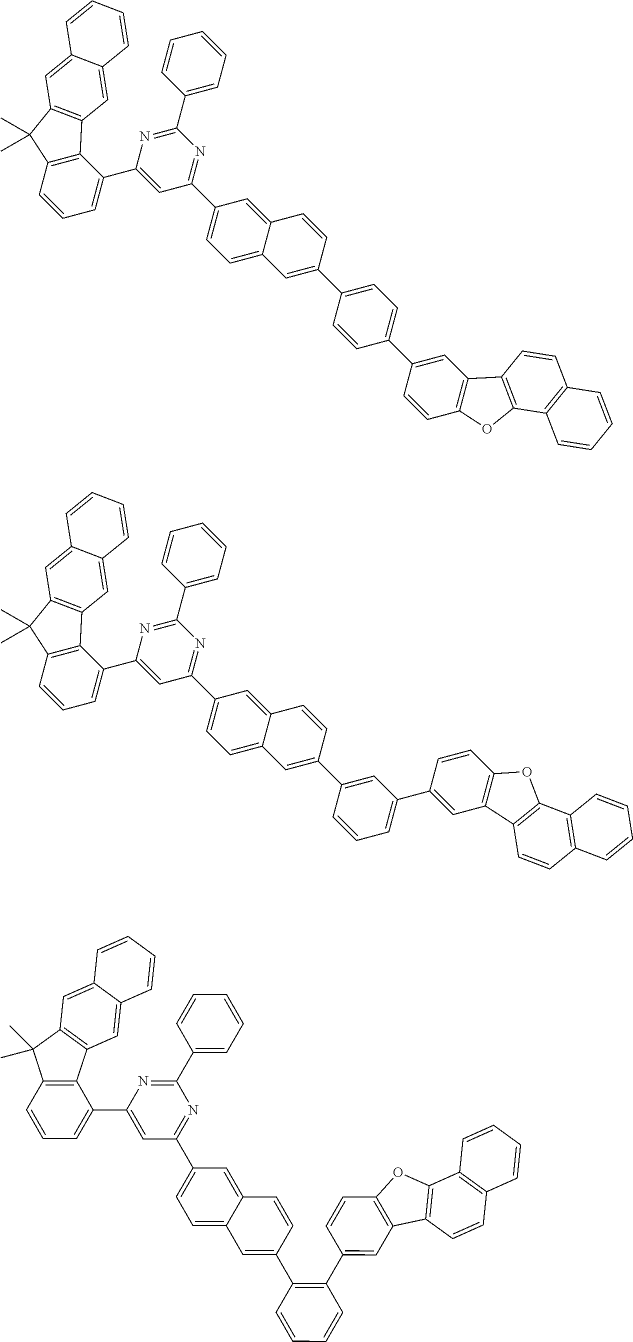 [SMILES: CC1(C)c2cc3ccccc3cc2-c2c(-c3cc(-c4ccc5cc(-c6ccc(-c7ccc8oc9c%10ccccc%10ccc9c8c7)cc6)ccc5c4)nc(-c4ccccc4)n3)cccc21 ; CC1(C)c2cc3ccccc3cc2-c2c(-c3cc(-c4ccc5cc(-c6cccc(-c7ccc8oc9c%10ccccc%10ccc9c8c7)c6)ccc5c4)nc(-c4ccccc4)n3)cccc21 ; CC1(C)c2cc3ccccc3cc2-c2c(-c3cc(-c4ccc5cc(-c6ccccc6-c6ccc7oc8c9ccccc9ccc8c7c6)ccc5c4)nc(-c4ccccc4)n3)cccc21]

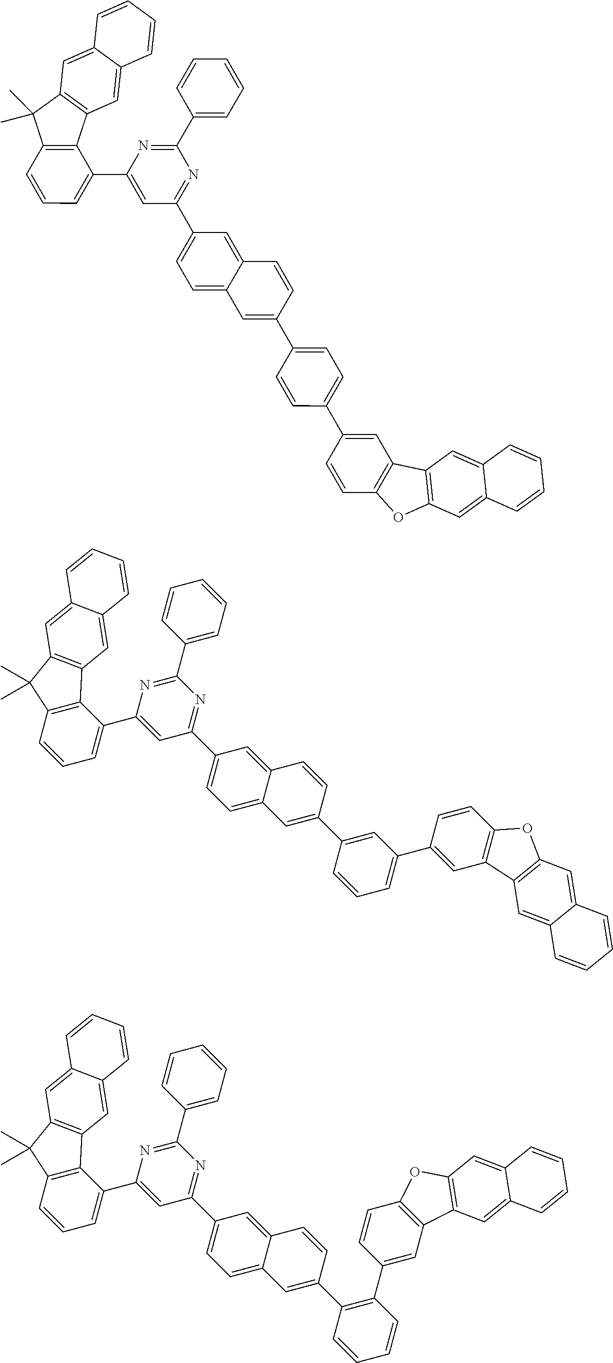 [SMILES: CC1(C)c2cc3ccccc3cc2-c2c(-c3cc(-c4ccc5cc(-c6ccc(-c7ccc8oc9cc%10ccccc%10cc9c8c7)cc6)ccc5c4)nc(-c4ccccc4)n3)cccc21 ; CC1(C)c2cc3ccccc3cc2-c2c(-c3cc(-c4ccc5cc(-c6cccc(-c7ccc8oc9cc%10ccccc%10cc9c8c7)c6)ccc5c4)nc(-c4ccccc4)n3)cccc21 ; CC1(C)c2cc3ccccc3cc2-c2c(-c3cc(-c4ccc5cc(-c6ccccc6-c6ccc7oc8cc9ccccc9cc8c7c6)ccc5c4)nc(-c4ccccc4)n3)cccc21]

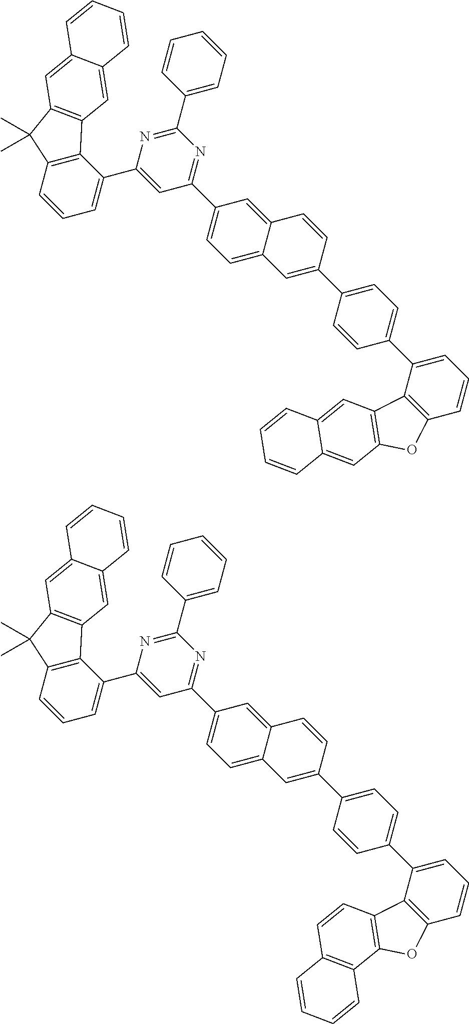 [SMILES: CC1(C)c2cc3ccccc3cc2-c2c(-c3cc(-c4ccc5cc(-c6ccc(-c7cccc8oc9cc%10ccccc%10cc9c78)cc6)ccc5c4)nc(-c4ccccc4)n3)cccc21 ; CC1(C)c2cc3ccccc3cc2-c2c(-c3cc(-c4ccc5cc(-c6ccc(-c7cccc8oc9c%10ccccc%10ccc9c78)cc6)ccc5c4)nc(-c4ccccc4)n3)cccc21]

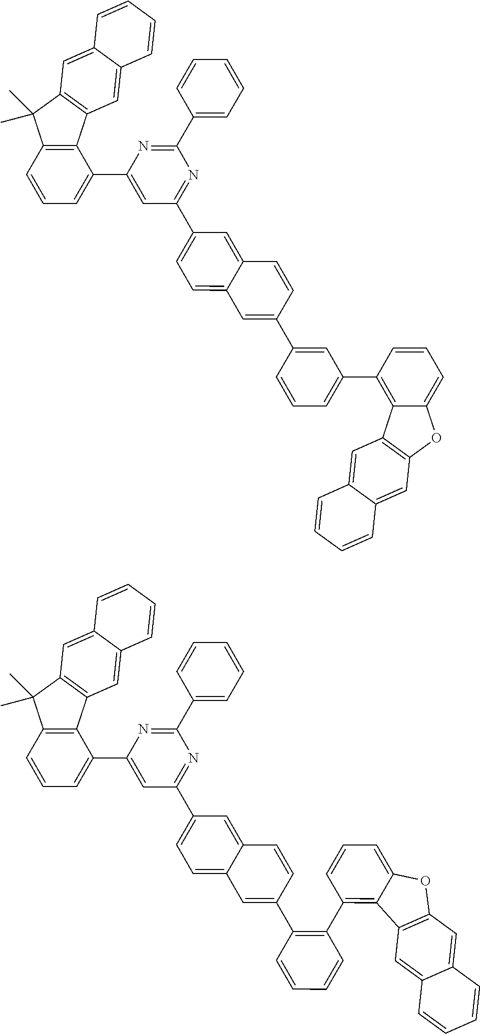 [SMILES: CC1(C)c2cc3ccccc3cc2-c2c(-c3cc(-c4ccc5cc(-c6cccc(-c7cccc8oc9cc%10ccccc%10cc9c78)c6)ccc5c4)nc(-c4ccccc4)n3)cccc21 ; CC1(C)c2cc3ccccc3cc2-c2c(-c3cc(-c4ccc5cc(-c6ccccc6-c6cccc7oc8cc9ccccc9cc8c67)ccc5c4)nc(-c4ccccc4)n3)cccc21]

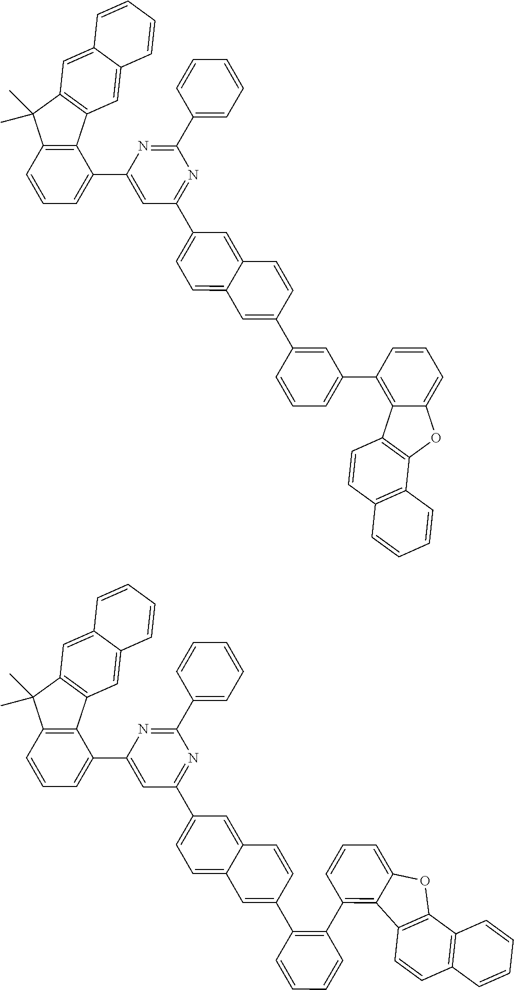 [SMILES: CC1(C)c2cc3ccccc3cc2-c2c(-c3cc(-c4ccc5cc(-c6cccc(-c7cccc8oc9c%10ccccc%10ccc9c78)c6)ccc5c4)nc(-c4ccccc4)n3)cccc21 ; CC1(C)c2cc3ccccc3cc2-c2c(-c3cc(-c4ccc5cc(-c6ccccc6-c6cccc7oc8c9ccccc9ccc8c67)ccc5c4)nc(-c4ccccc4)n3)cccc21]

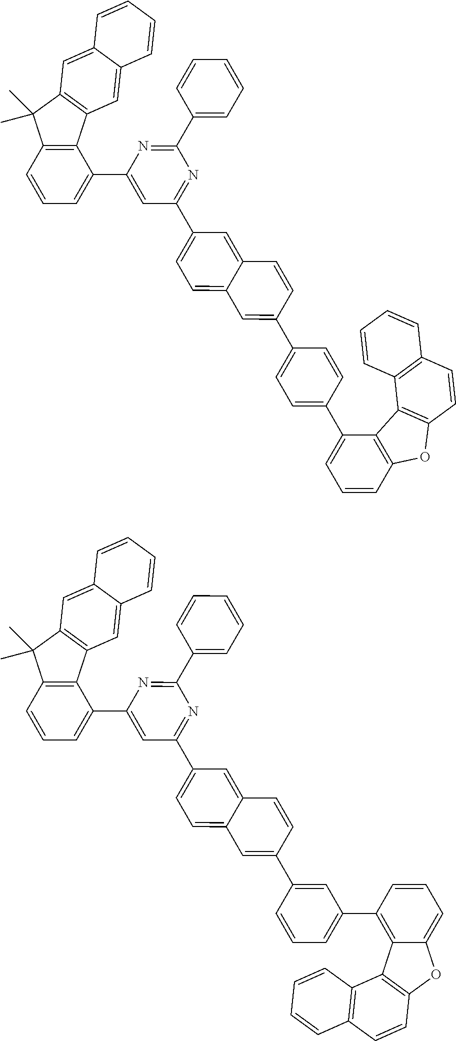 [SMILES: CC1(C)c2cc3ccccc3cc2-c2c(-c3cc(-c4ccc5cc(-c6ccc(-c7cccc8oc9ccc%10ccccc%10c9c78)cc6)ccc5c4)nc(-c4ccccc4)n3)cccc21 ; CC1(C)c2cc3ccccc3cc2-c2c(-c3cc(-c4ccc5cc(-c6cccc(-c7cccc8oc9ccc%10ccccc%10c9c78)c6)ccc5c4)nc(-c4ccccc4)n3)cccc21]

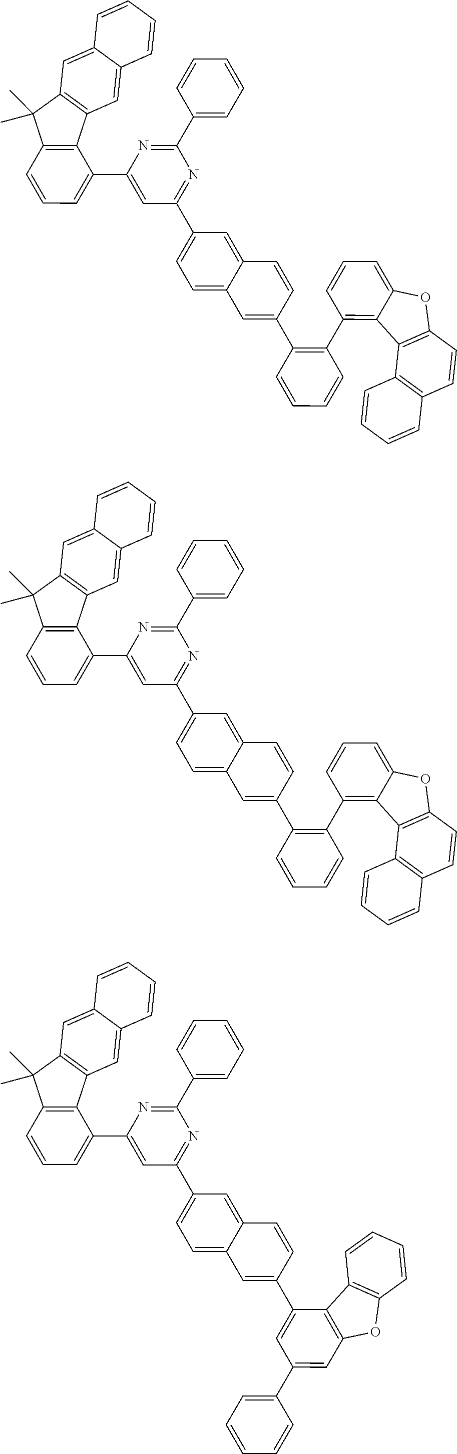 [SMILES: CC1(C)c2cc3ccccc3cc2-c2c(-c3cc(-c4ccc5cc(-c6ccccc6-c6cccc7oc8ccc9ccccc9c8c67)ccc5c4)nc(-c4ccccc4)n3)cccc21 ; CC1(C)c2cc3ccccc3cc2-c2c(-c3cc(-c4ccc5cc(-c6ccccc6-c6cccc7oc8ccc9ccccc9c8c67)ccc5c4)nc(-c4ccccc4)n3)cccc21 ; CC1(C)c2cc3ccccc3cc2-c2c(-c3cc(-c4ccc5cc(-c6cc(-c7ccccc7)cc7oc8ccccc8c67)ccc5c4)nc(-c4ccccc4)n3)cccc21]

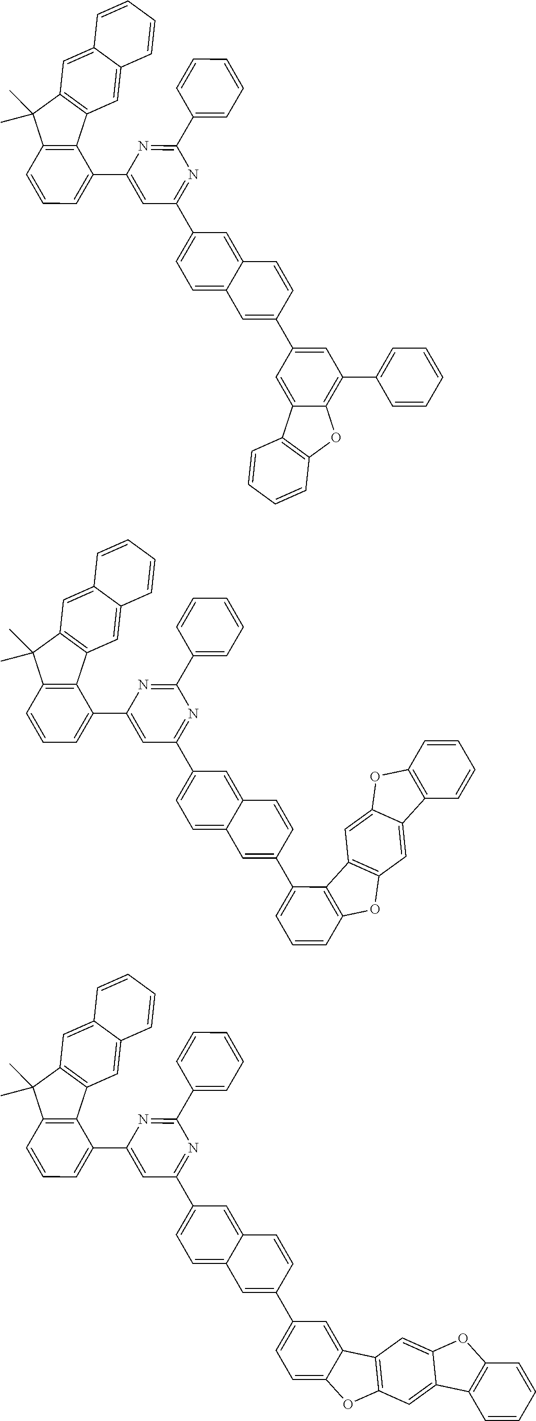 [SMILES: CC1(C)c2cc3ccccc3cc2-c2c(-c3cc(-c4ccc5cc(-c6cc(-c7ccccc7)c7oc8ccccc8c7c6)ccc5c4)nc(-c4ccccc4)n3)cccc21 ; CC1(C)c2cc3ccccc3cc2-c2c(-c3cc(-c4ccc5cc(-c6cccc7oc8cc9c(cc8c67)oc6ccccc69)ccc5c4)nc(-c4ccccc4)n3)cccc21 ; CC1(C)c2cc3ccccc3cc2-c2c(-c3cc(-c4ccc5cc(-c6ccc7oc8cc9c(cc8c7c6)oc6ccccc69)ccc5c4)nc(-c4ccccc4)n3)cccc21]

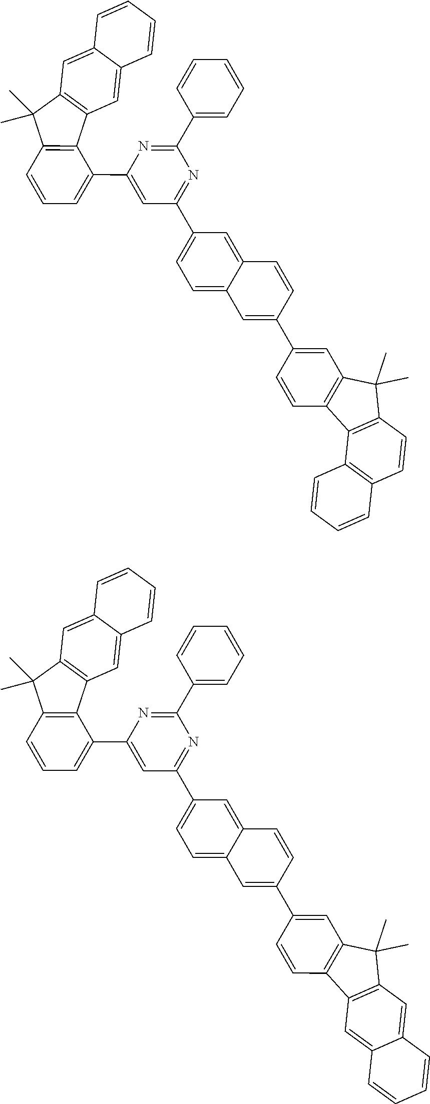

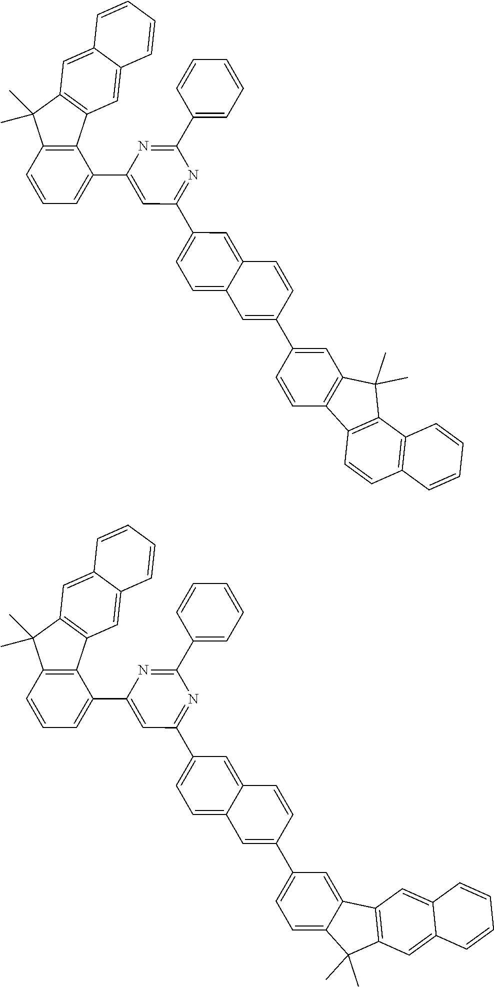 [SMILES: CC1(C)c2cc3ccccc3cc2-c2c(-c3cc(-c4ccc5cc(-c6ccc7c(c6)C(C)(C)c6c-7ccc7ccccc67)ccc5c4)nc(-c4ccccc4)n3)cccc21 ; CC1(C)c2ccc(-c3ccc4cc(-c5cc(-c6cccc7c6-c6cc8ccccc8cc6C7(C)C)nc(-c6ccccc6)n5)ccc4c3)cc2-c2cc3ccccc3cc21]

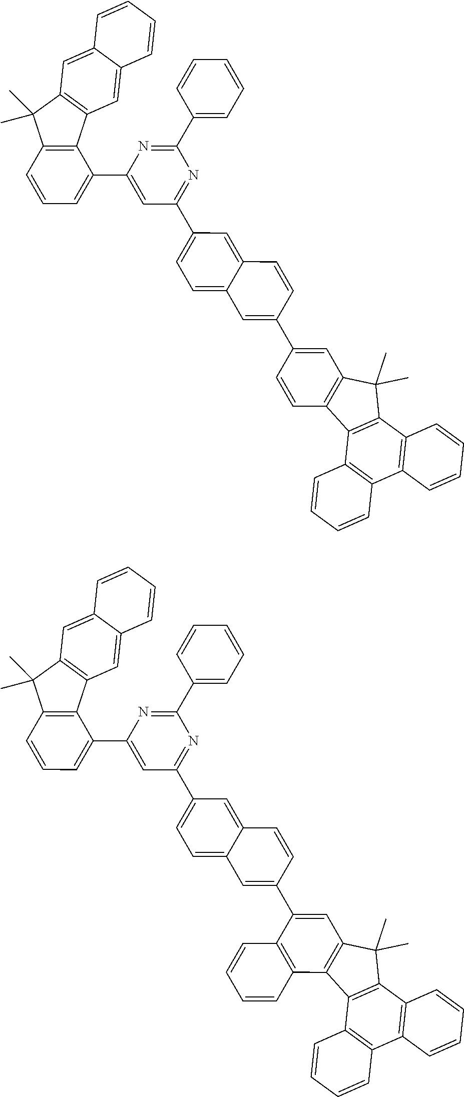 [SMILES: CC1(C)c2cc3ccccc3cc2-c2c(-c3cc(-c4ccc5cc(-c6ccc7c(c6)C(C)(C)c6c-7c7ccccc7c7ccccc67)ccc5c4)nc(-c4ccccc4)n3)cccc21 ; CC1(C)c2cc3ccccc3cc2-c2c(-c3cc(-c4ccc5cc(-c6cc7c(c8ccccc68)-c6c(c8ccccc8c8ccccc68)C7(C)C)ccc5c4)nc(-c4ccccc4)n3)cccc21]

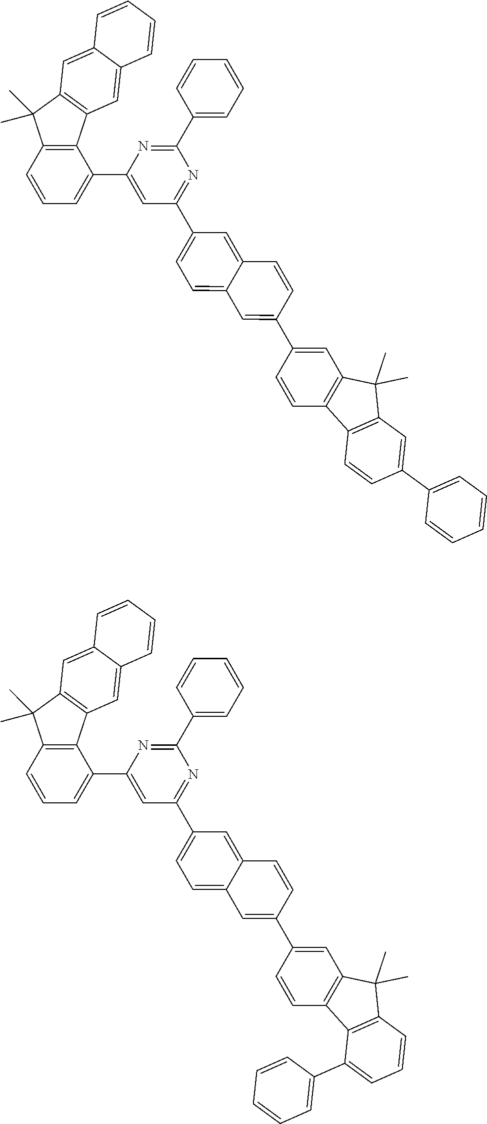

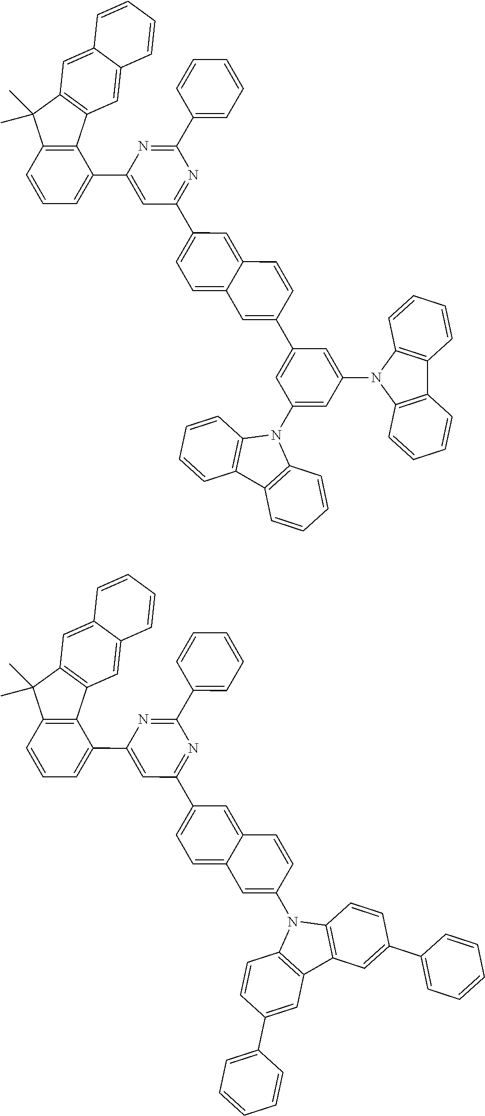 [SMILES: CC1(C)c2cc3ccccc3cc2-c2c(-c3cc(-c4ccc5cc(-c6cc(-n7c8ccccc8c8ccccc87)cc(-n7c8ccccc8c8ccccc87)c6)ccc5c4)nc(-c4ccccc4)n3)cccc21 ; CC1(C)c2cc3ccccc3cc2-c2c(-c3cc(-c4ccc5cc(-n6c7ccc(-c8ccccc8)cc7c7cc(-c8ccccc8)ccc76)ccc5c4)nc(-c4ccccc4)n3)cccc21]

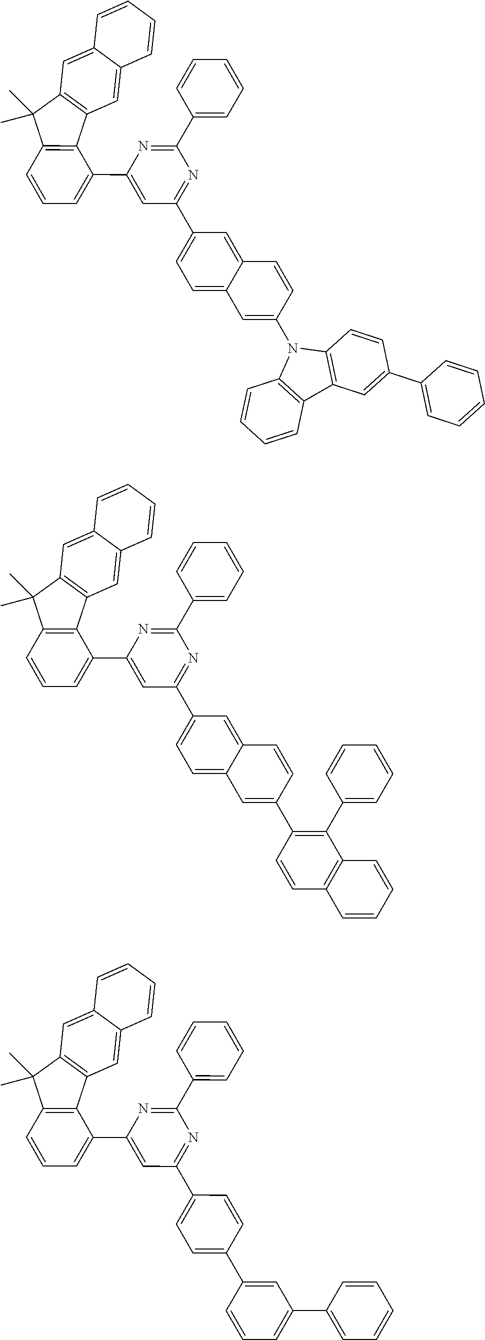 [SMILES: CC1(C)c2cc3ccccc3cc2-c2c(-c3cc(-c4ccc5cc(-n6c7ccccc7c7cc(-c8ccccc8)ccc76)ccc5c4)nc(-c4ccccc4)n3)cccc21 ; CC1(C)c2cc3ccccc3cc2-c2c(-c3cc(-c4ccc5cc(-c6ccc7ccccc7c6-c6ccccc6)ccc5c4)nc(-c4ccccc4)n3)cccc21 ; CC1(C)c2cc3ccccc3cc2-c2c(-c3cc(-c4ccc(-c5cccc(-c6ccccc6)c5)cc4)nc(-c4ccccc4)n3)cccc21]

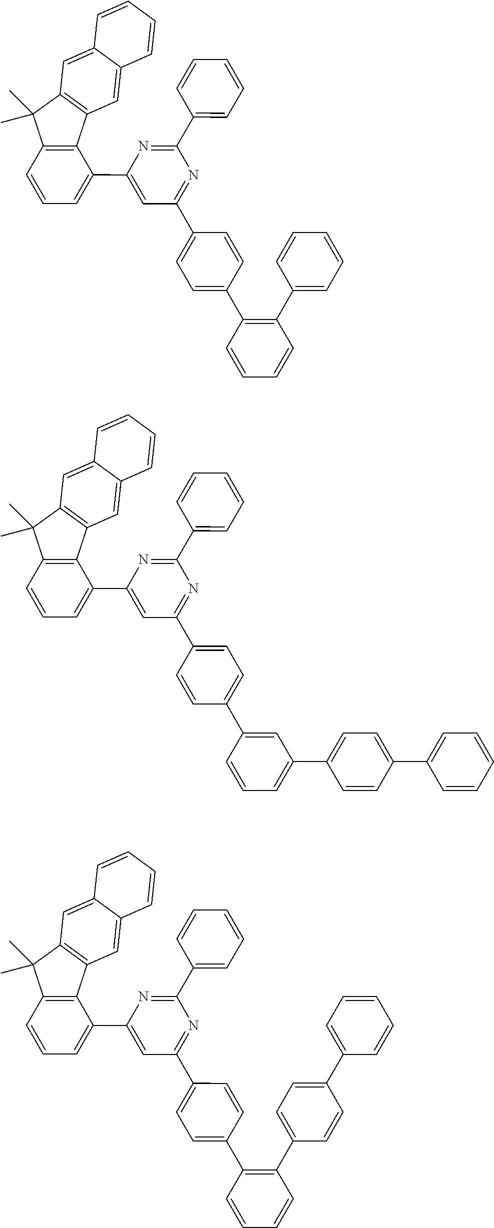 [SMILES: CC1(C)c2cc3ccccc3cc2-c2c(-c3cc(-c4ccc(-c5ccccc5-c5ccccc5)cc4)nc(-c4ccccc4)n3)cccc21 ; CC1(C)c2cc3ccccc3cc2-c2c(-c3cc(-c4ccc(-c5cccc(-c6ccc(-c7ccccc7)cc6)c5)cc4)nc(-c4ccccc4)n3)cccc21 ; CC1(C)c2cc3ccccc3cc2-c2c(-c3cc(-c4ccc(-c5ccccc5-c5ccc(-c6ccccc6)cc5)cc4)nc(-c4ccccc4)n3)cccc21]

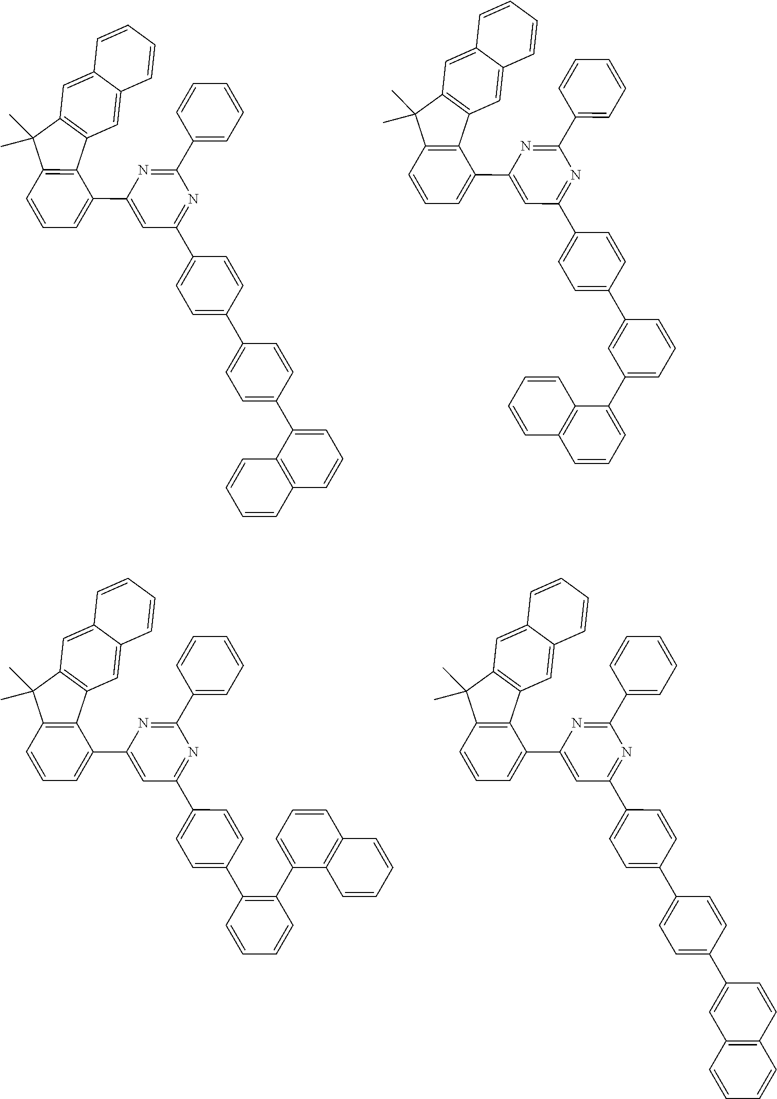 [SMILES: CC1(C)c2cc3ccccc3cc2-c2c(-c3cc(-c4ccc(-c5ccc(-c6cccc7ccccc67)cc5)cc4)nc(-c4ccccc4)n3)cccc21 ; CC1(C)c2cc3ccccc3cc2-c2c(-c3cc(-c4ccc(-c5cccc(-c6cccc7ccccc67)c5)cc4)nc(-c4ccccc4)n3)cccc21 ; CC1(C)c2cc3ccccc3cc2-c2c(-c3cc(-c4ccc(-c5ccccc5-c5cccc6ccccc56)cc4)nc(-c4ccccc4)n3)cccc21 ; CC1(C)c2cc3ccccc3cc2-c2c(-c3cc(-c4ccc(-c5ccc(-c6ccc7ccccc7c6)cc5)cc4)nc(-c4ccccc4)n3)cccc21]

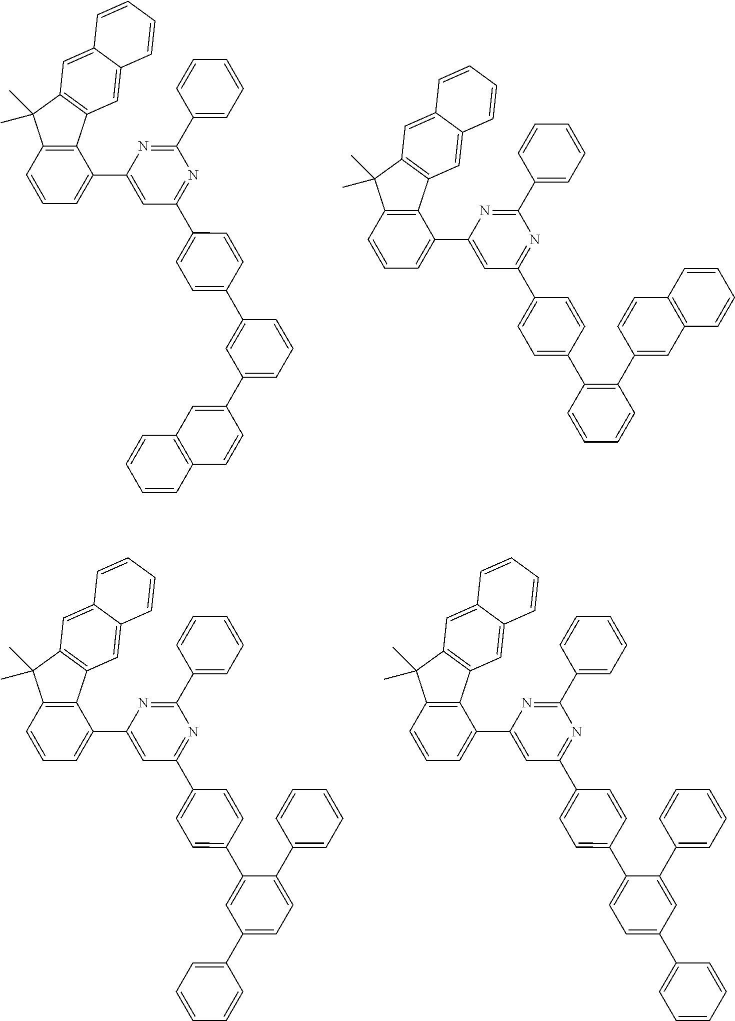 [SMILES: CC1(C)c2cc3ccccc3cc2-c2c(-c3cc(-c4ccc(-c5cccc(-c6ccc7ccccc7c6)c5)cc4)nc(-c4ccccc4)n3)cccc21 ; CC1(C)c2cc3ccccc3cc2-c2c(-c3cc(-c4ccc(-c5ccccc5-c5ccc6ccccc6c5)cc4)nc(-c4ccccc4)n3)cccc21 ; CC1(C)c2cc3ccccc3cc2-c2c(-c3cc(-c4ccc(-c5cc(-c6ccccc6)ccc5-c5ccccc5)cc4)nc(-c4ccccc4)n3)cccc21 ; CC1(C)c2cc3ccccc3cc2-c2c(-c3cc(-c4ccc(-c5ccc(-c6ccccc6)cc5-c5ccccc5)cc4)nc(-c4ccccc4)n3)cccc21]

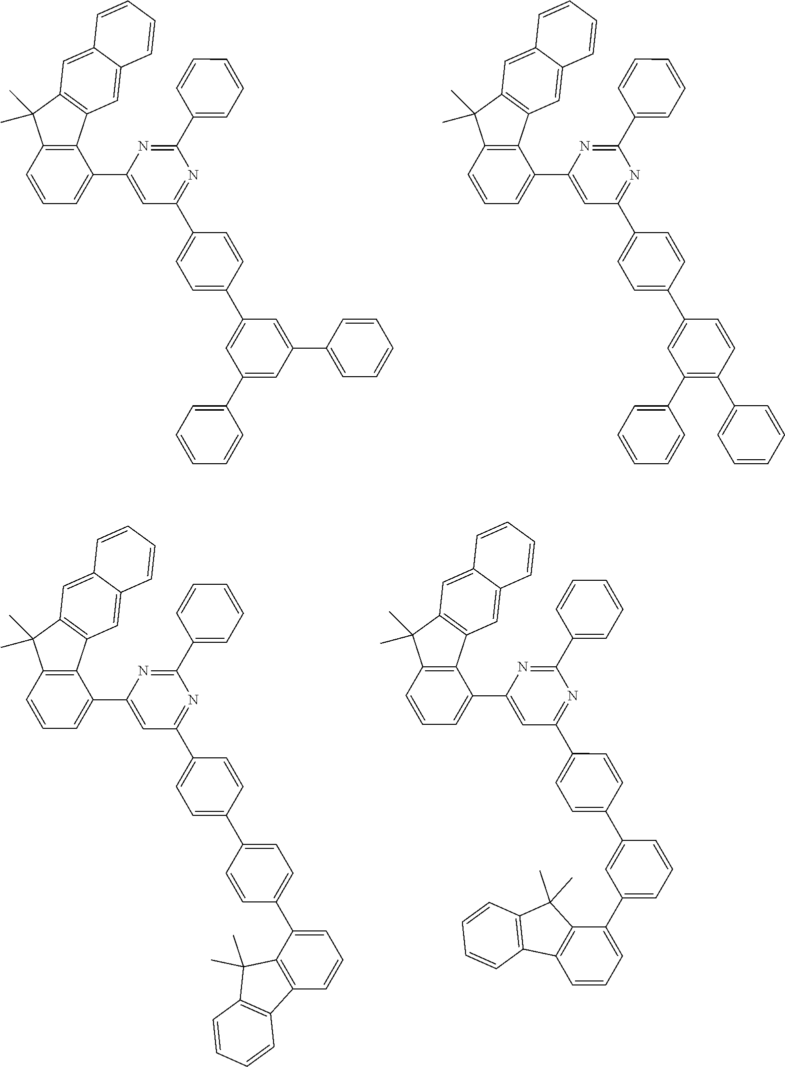 [SMILES: CC1(C)c2cc3ccccc3cc2-c2c(-c3cc(-c4ccc(-c5cc(-c6ccccc6)cc(-c6ccccc6)c5)cc4)nc(-c4ccccc4)n3)cccc21 ; CC1(C)c2cc3ccccc3cc2-c2c(-c3cc(-c4ccc(-c5ccc(-c6ccccc6)c(-c6ccccc6)c5)cc4)nc(-c4ccccc4)n3)cccc21 ; CC1(C)c2cc3ccccc3cc2-c2c(-c3cc(-c4ccc(-c5ccc(-c6cccc7c6C(C)(C)c6ccccc6-7)cc5)cc4)nc(-c4ccccc4)n3)cccc21 ; CC1(C)c2cc3ccccc3cc2-c2c(-c3cc(-c4ccc(-c5cccc(-c6cccc7c6C(C)(C)c6ccccc6-7)c5)cc4)nc(-c4ccccc4)n3)cccc21]

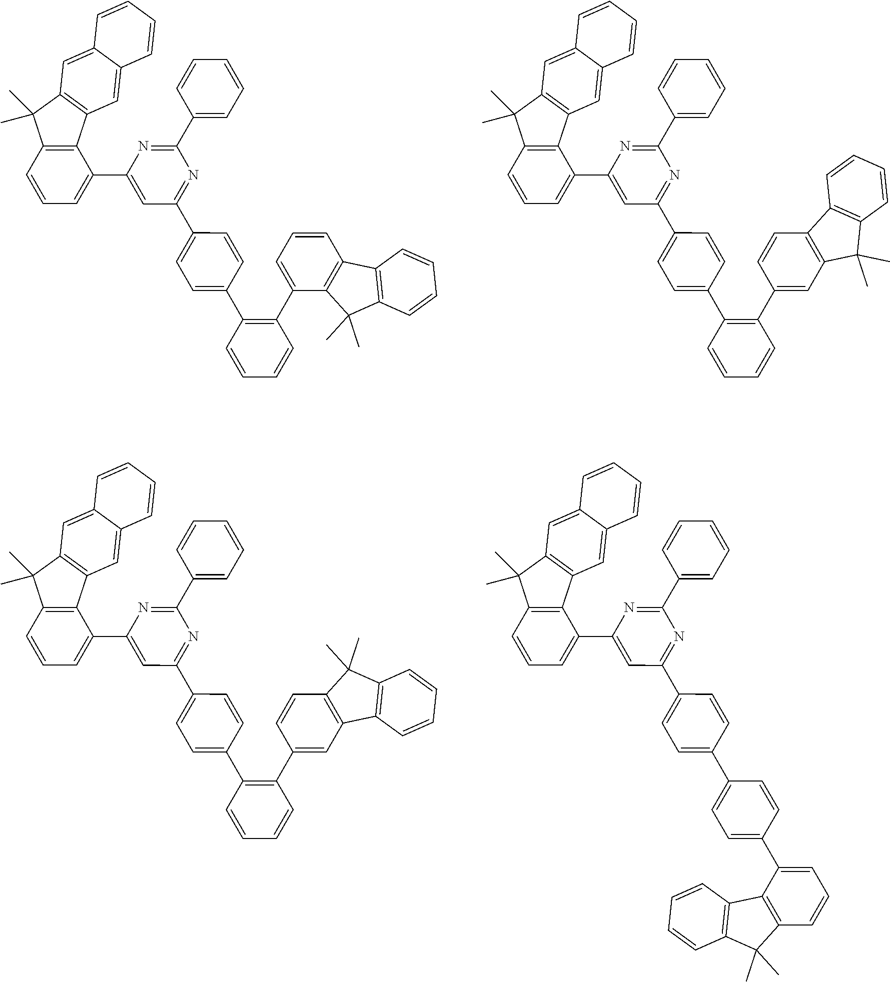

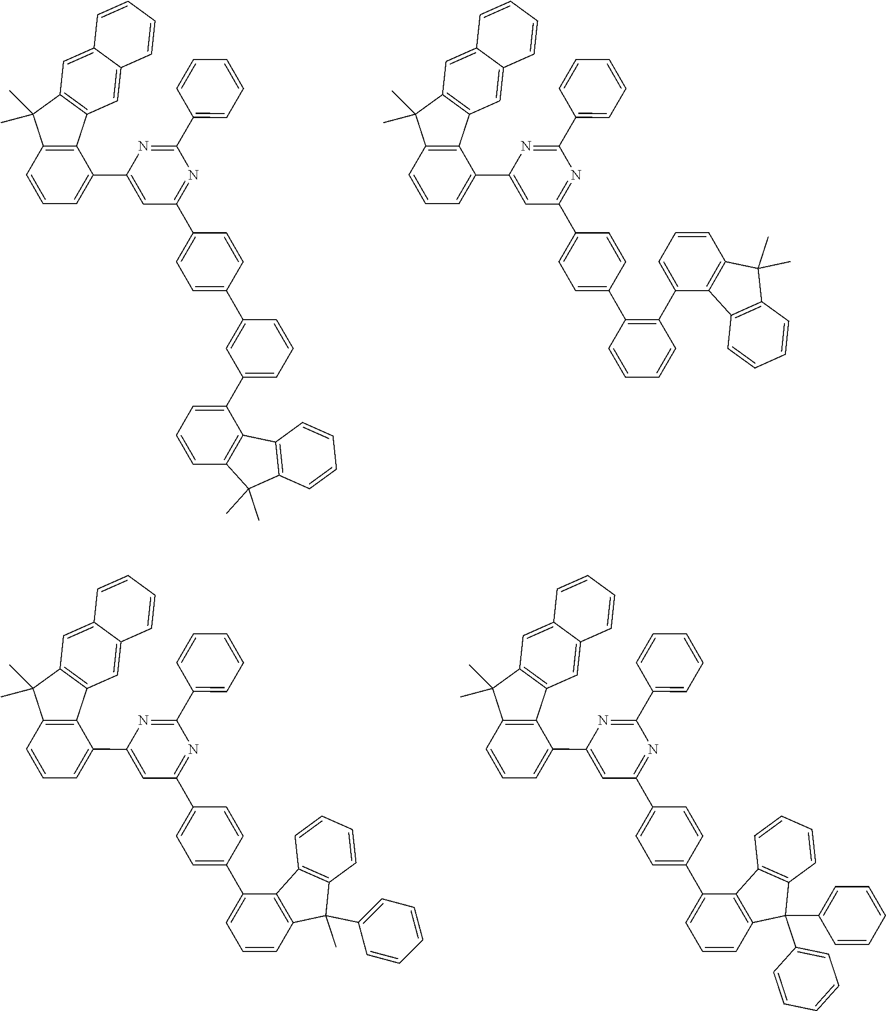

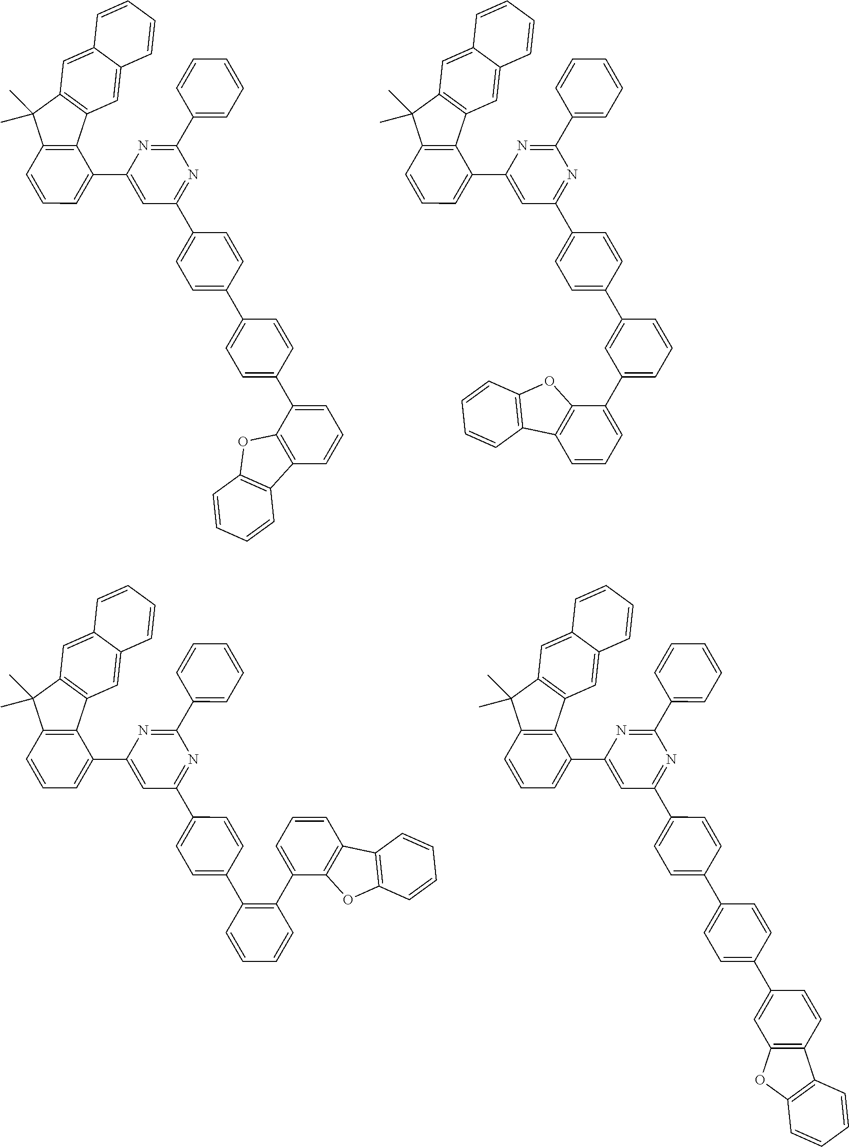 [SMILES: CC1(C)c2cc3ccccc3cc2-c2c(-c3cc(-c4ccc(-c5ccc(-c6cccc7c6oc6ccccc67)cc5)cc4)nc(-c4ccccc4)n3)cccc21 ; CC1(C)c2cc3ccccc3cc2-c2c(-c3cc(-c4ccc(-c5cccc(-c6cccc7c6oc6ccccc67)c5)cc4)nc(-c4ccccc4)n3)cccc21 ; CC1(C)c2cc3ccccc3cc2-c2c(-c3cc(-c4ccc(-c5ccccc5-c5cccc6c5oc5ccccc56)cc4)nc(-c4ccccc4)n3)cccc21 ; CC1(C)c2cc3ccccc3cc2-c2c(-c3cc(-c4ccc(-c5ccc(-c6ccc7c(c6)oc6ccccc67)cc5)cc4)nc(-c4ccccc4)n3)cccc21]

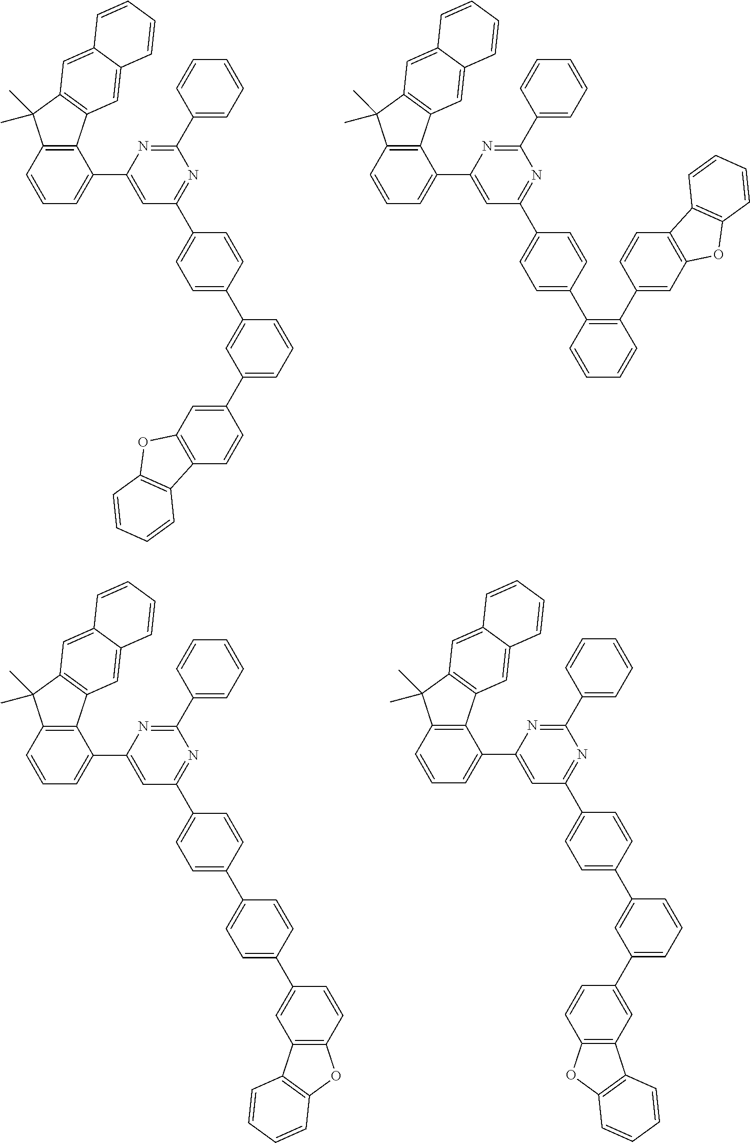

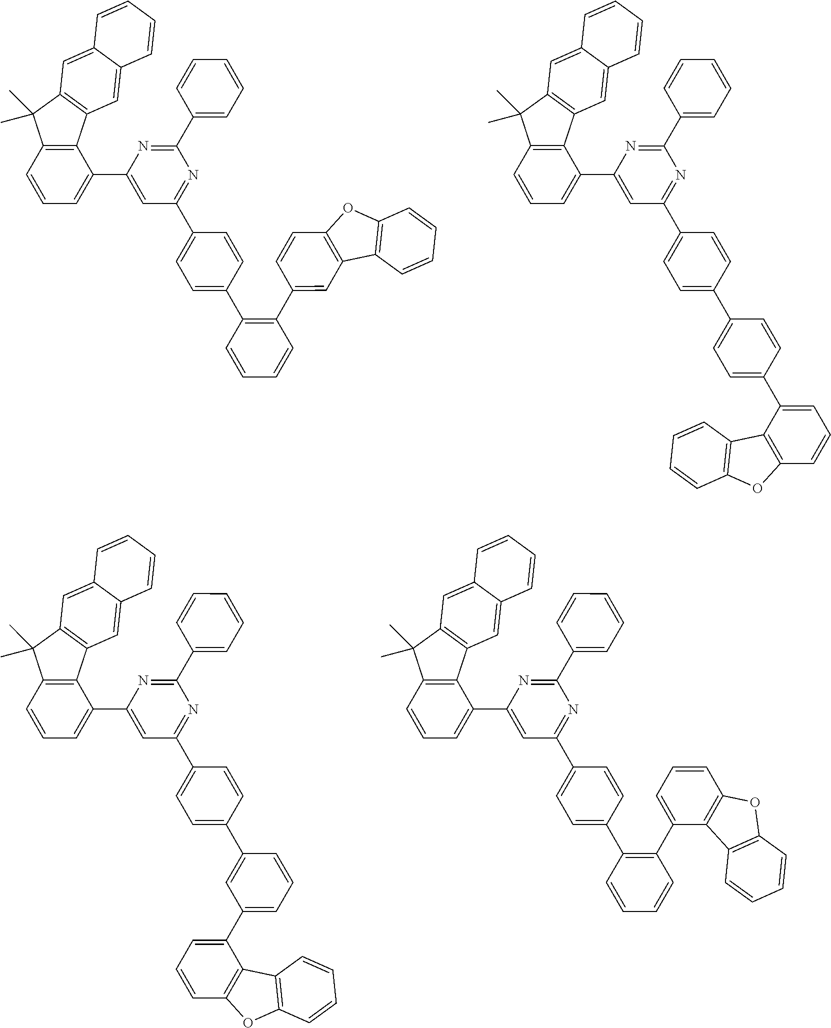 [SMILES: CC1(C)c2cc3ccccc3cc2-c2c(-c3cc(-c4ccc(-c5ccccc5-c5ccc6oc7ccccc7c6c5)cc4)nc(-c4ccccc4)n3)cccc21 ; CC1(C)c2cc3ccccc3cc2-c2c(-c3cc(-c4ccc(-c5ccc(-c6cccc7oc8ccccc8c67)cc5)cc4)nc(-c4ccccc4)n3)cccc21 ; CC1(C)c2cc3ccccc3cc2-c2c(-c3cc(-c4ccc(-c5cccc(-c6cccc7oc8ccccc8c67)c5)cc4)nc(-c4ccccc4)n3)cccc21 ; CC1(C)c2cc3ccccc3cc2-c2c(-c3cc(-c4ccc(-c5ccccc5-c5cccc6oc7ccccc7c56)cc4)nc(-c4ccccc4)n3)cccc21]

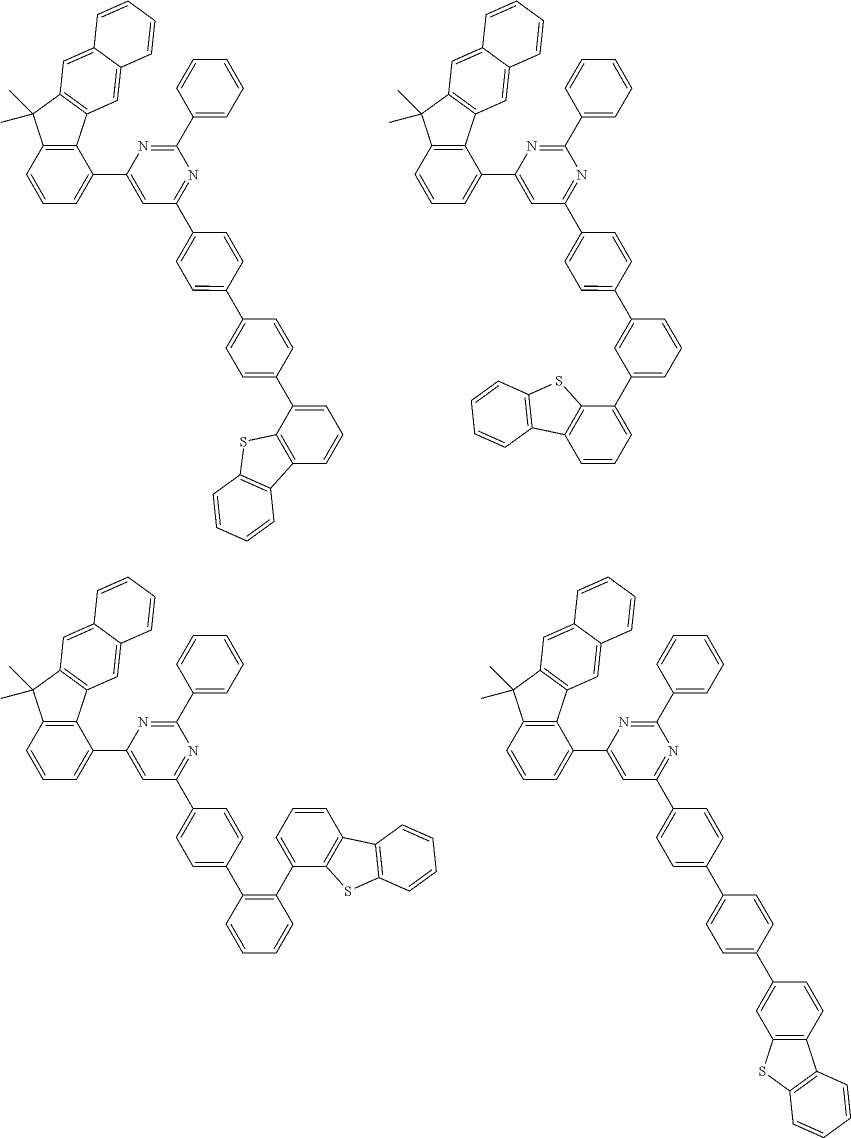 [SMILES: CC1(C)c2cc3ccccc3cc2-c2c(-c3cc(-c4ccc(-c5ccc(-c6cccc7c6sc6ccccc67)cc5)cc4)nc(-c4ccccc4)n3)cccc21 ; CC1(C)c2cc3ccccc3cc2-c2c(-c3cc(-c4ccc(-c5cccc(-c6cccc7c6sc6ccccc67)c5)cc4)nc(-c4ccccc4)n3)cccc21 ; CC1(C)c2cc3ccccc3cc2-c2c(-c3cc(-c4ccc(-c5ccccc5-c5cccc6c5sc5ccccc56)cc4)nc(-c4ccccc4)n3)cccc21 ; CC1(C)c2cc3ccccc3cc2-c2c(-c3cc(-c4ccc(-c5ccc(-c6ccc7c(c6)sc6ccccc67)cc5)cc4)nc(-c4ccccc4)n3)cccc21]

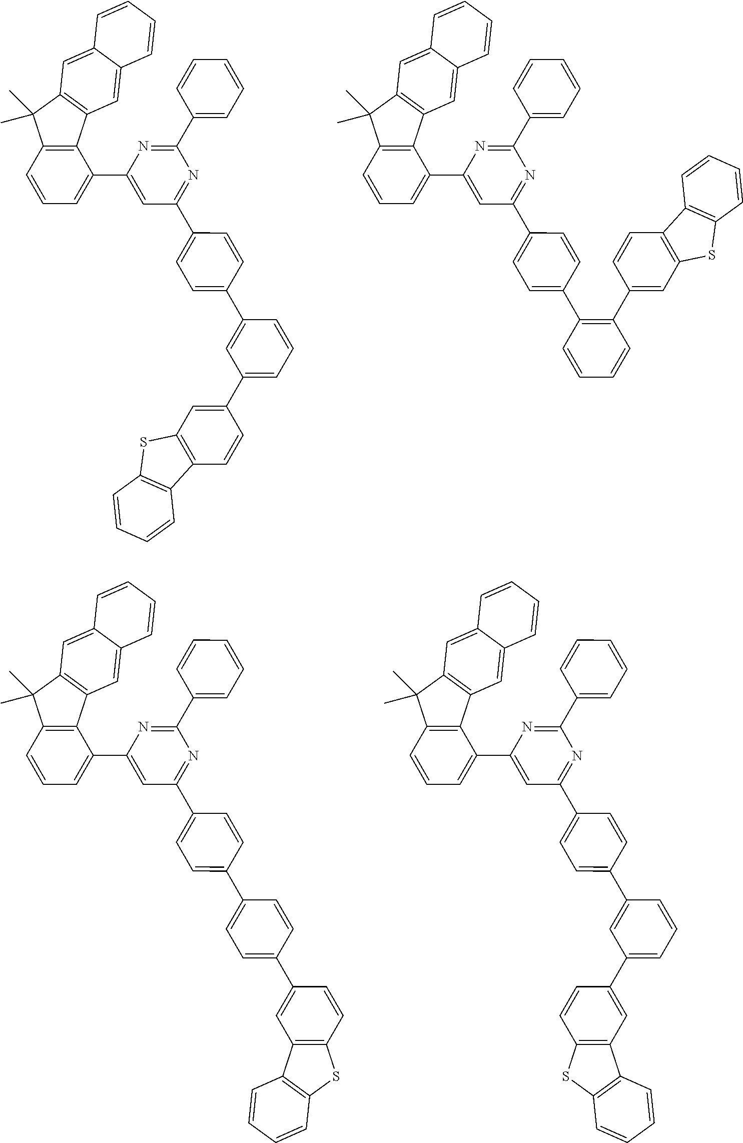

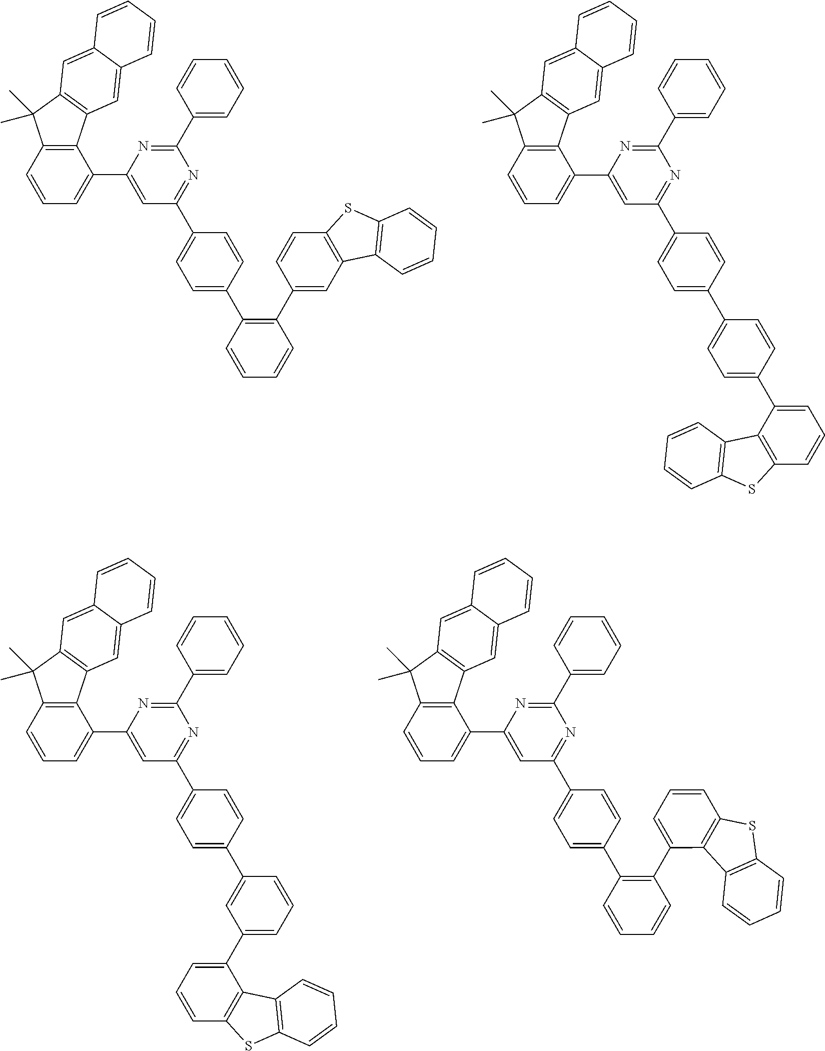 [SMILES: CC1(C)c2cc3ccccc3cc2-c2c(-c3cc(-c4ccc(-c5ccccc5-c5ccc6sc7ccccc7c6c5)cc4)nc(-c4ccccc4)n3)cccc21 ; CC1(C)c2cc3ccccc3cc2-c2c(-c3cc(-c4ccc(-c5ccc(-c6cccc7sc8ccccc8c67)cc5)cc4)nc(-c4ccccc4)n3)cccc21 ; CC1(C)c2cc3ccccc3cc2-c2c(-c3cc(-c4ccc(-c5cccc(-c6cccc7sc8ccccc8c67)c5)cc4)nc(-c4ccccc4)n3)cccc21 ; CC1(C)c2cc3ccccc3cc2-c2c(-c3cc(-c4ccc(-c5ccccc5-c5cccc6sc7ccccc7c56)cc4)nc(-c4ccccc4)n3)cccc21]

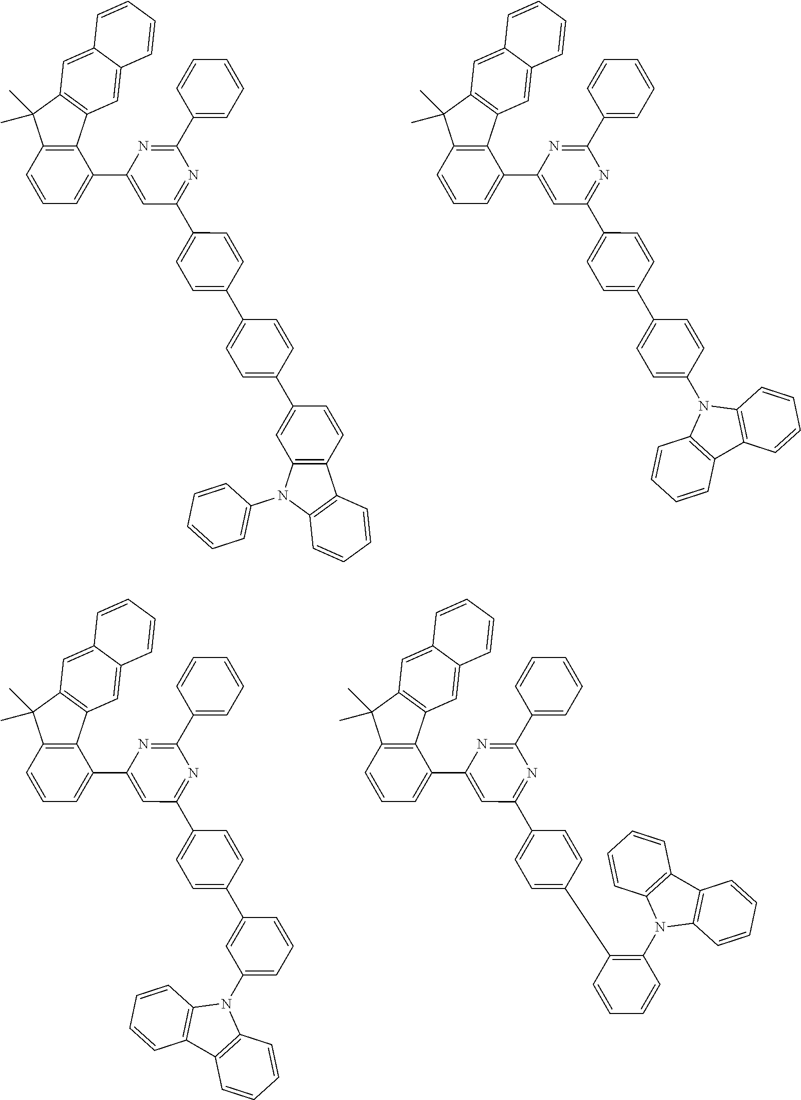 [SMILES: CC1(C)c2cc3ccccc3cc2-c2c(-c3cc(-c4ccc(-c5ccc(-c6ccc7c8ccccc8n(-c8ccccc8)c7c6)cc5)cc4)nc(-c4ccccc4)n3)cccc21 ; CC1(C)c2cc3ccccc3cc2-c2c(-c3cc(-c4ccc(-c5ccc(-n6c7ccccc7c7ccccc76)cc5)cc4)nc(-c4ccccc4)n3)cccc21 ; CC1(C)c2cc3ccccc3cc2-c2c(-c3cc(-c4ccc(-c5cccc(-n6c7ccccc7c7ccccc76)c5)cc4)nc(-c4ccccc4)n3)cccc21 ; CC1(C)c2cc3ccccc3cc2-c2c(-c3cc(-c4ccc(-c5ccccc5-n5c6ccccc6c6ccccc65)cc4)nc(-c4ccccc4)n3)cccc21]

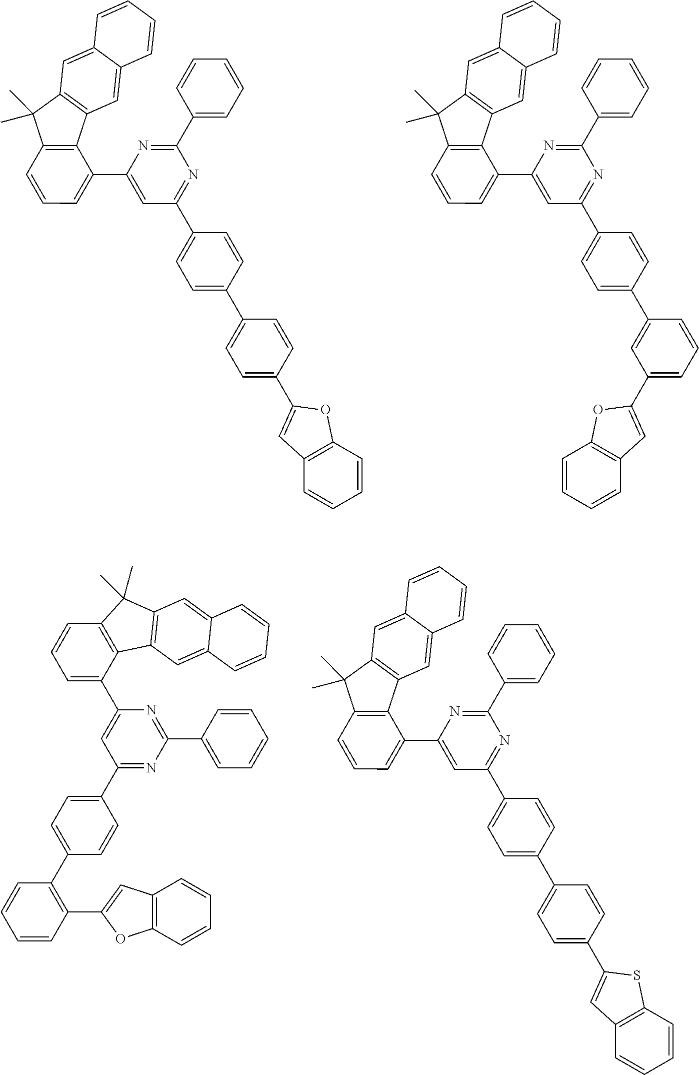 [SMILES: CC1(C)c2cc3ccccc3cc2-c2c(-c3cc(-c4ccc(-c5ccc(-c6cc7ccccc7o6)cc5)cc4)nc(-c4ccccc4)n3)cccc21 ; CC1(C)c2cc3ccccc3cc2-c2c(-c3cc(-c4ccc(-c5cccc(-c6cc7ccccc7o6)c5)cc4)nc(-c4ccccc4)n3)cccc21 ; CC1(C)c2cc3ccccc3cc2-c2c(-c3cc(-c4ccc(-c5ccccc5-c5cc6ccccc6o5)cc4)nc(-c4ccccc4)n3)cccc21 ; CC1(C)c2cc3ccccc3cc2-c2c(-c3cc(-c4ccc(-c5ccc(-c6cc7ccccc7s6)cc5)cc4)nc(-c4ccccc4)n3)cccc21]

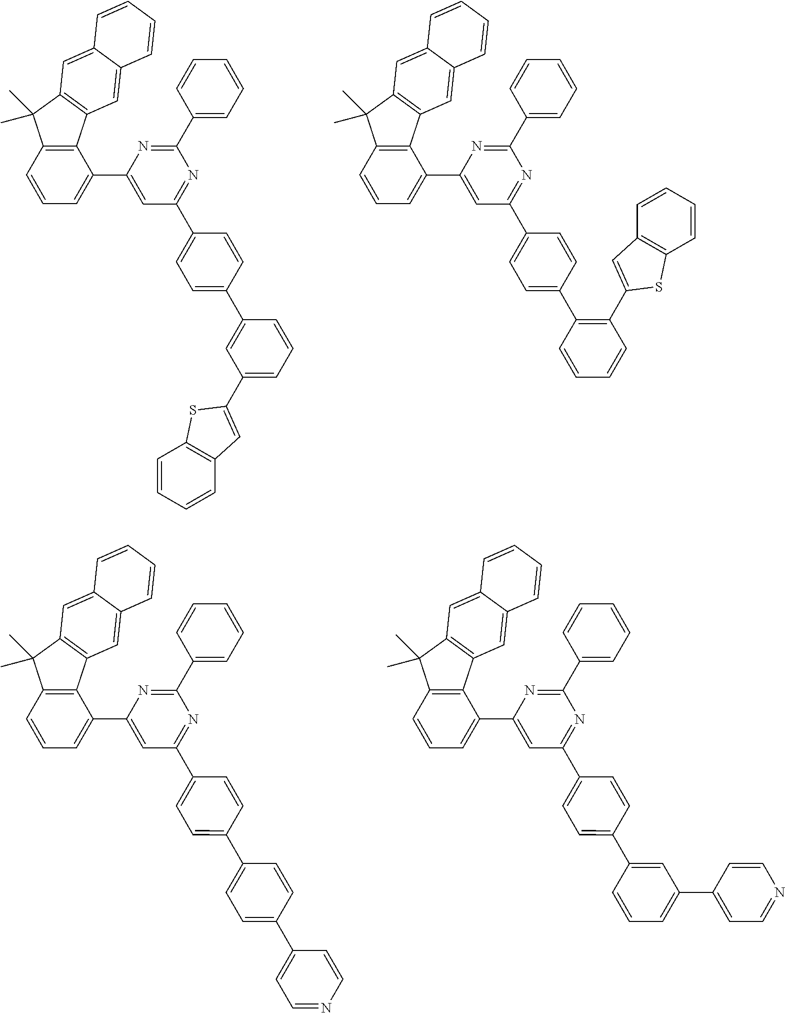 [SMILES: CC1(C)c2cc3ccccc3cc2-c2c(-c3cc(-c4ccc(-c5cccc(-c6cc7ccccc7s6)c5)cc4)nc(-c4ccccc4)n3)cccc21 ; CC1(C)c2cc3ccccc3cc2-c2c(-c3cc(-c4ccc(-c5ccccc5-c5cc6ccccc6s5)cc4)nc(-c4ccccc4)n3)cccc21 ; CC1(C)c2cc3ccccc3cc2-c2c(-c3cc(-c4ccc(-c5ccc(-c6ccncc6)cc5)cc4)nc(-c4ccccc4)n3)cccc21 ; CC1(C)c2cc3ccccc3cc2-c2c(-c3cc(-c4ccc(-c5cccc(-c6ccncc6)c5)cc4)nc(-c4ccccc4)n3)cccc21]

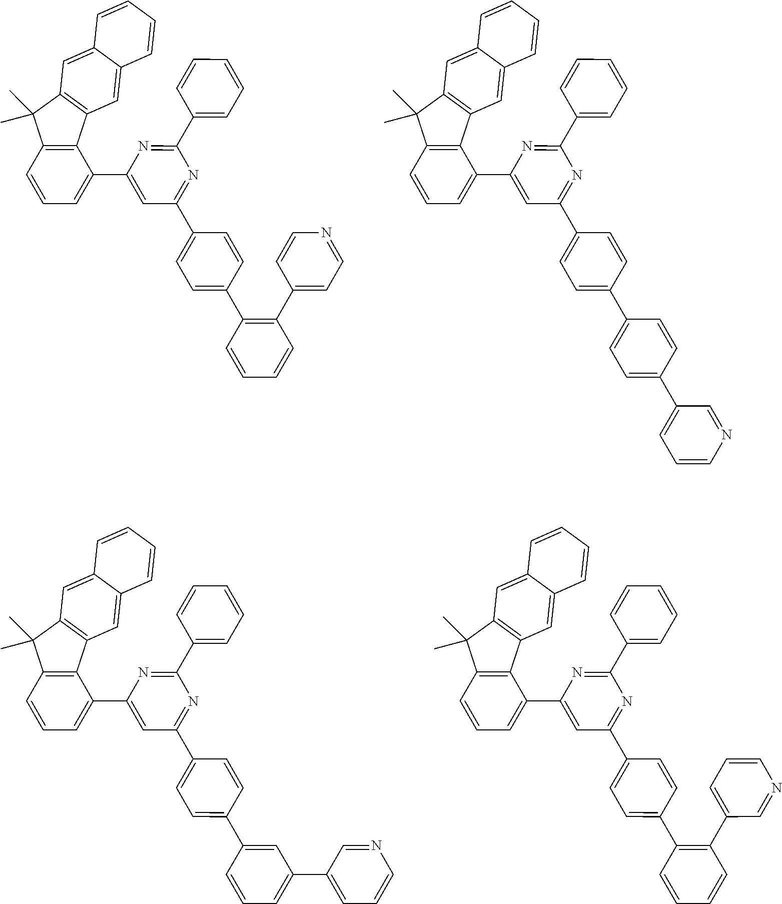 [SMILES: CC1(C)c2cc3ccccc3cc2-c2c(-c3cc(-c4ccc(-c5ccccc5-c5ccncc5)cc4)nc(-c4ccccc4)n3)cccc21 ; CC1(C)c2cc3ccccc3cc2-c2c(-c3cc(-c4ccc(-c5ccc(-c6cccnc6)cc5)cc4)nc(-c4ccccc4)n3)cccc21 ; CC1(C)c2cc3ccccc3cc2-c2c(-c3cc(-c4ccc(-c5cccc(-c6cccnc6)c5)cc4)nc(-c4ccccc4)n3)cccc21 ; CC1(C)c2cc3ccccc3cc2-c2c(-c3cc(-c4ccc(-c5ccccc5-c5cccnc5)cc4)nc(-c4ccccc4)n3)cccc21]

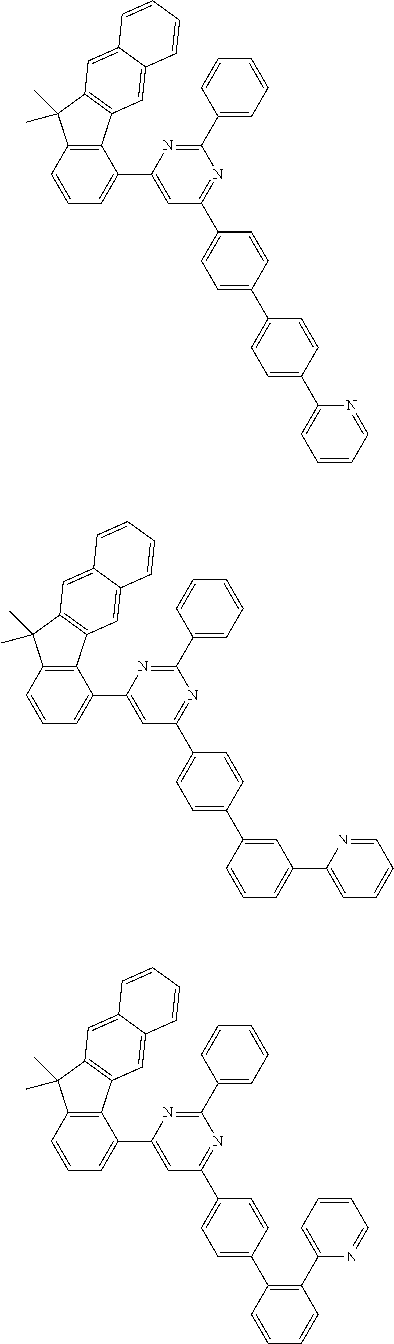 [SMILES: CC1(C)c2cc3ccccc3cc2-c2c(-c3cc(-c4ccc(-c5ccc(-c6ccccn6)cc5)cc4)nc(-c4ccccc4)n3)cccc21 ; CC1(C)c2cc3ccccc3cc2-c2c(-c3cc(-c4ccc(-c5cccc(-c6ccccn6)c5)cc4)nc(-c4ccccc4)n3)cccc21 ; CC1(C)c2cc3ccccc3cc2-c2c(-c3cc(-c4ccc(-c5ccccc5-c5ccccn5)cc4)nc(-c4ccccc4)n3)cccc21]

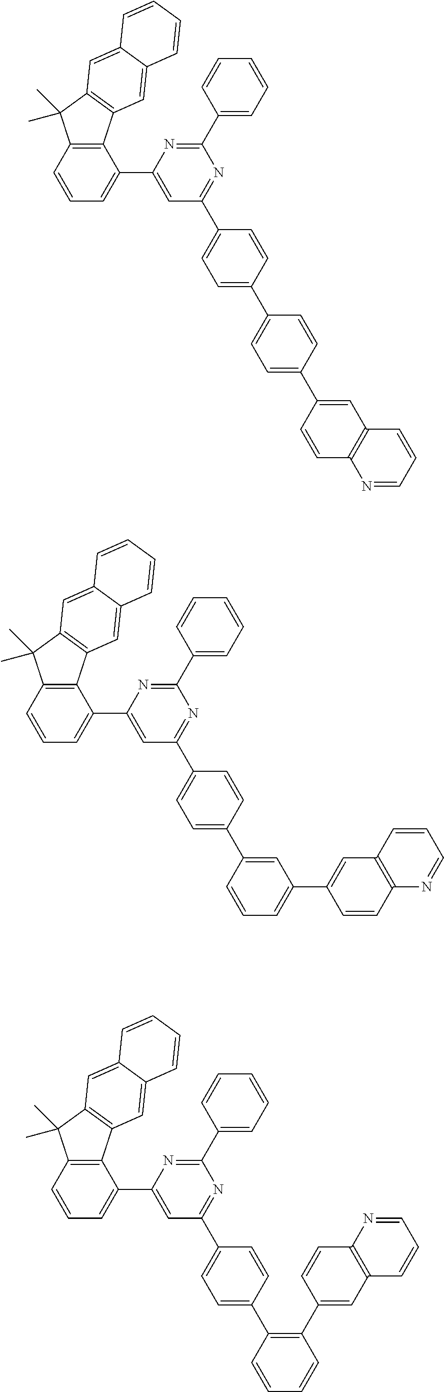 [SMILES: CC1(C)c2cc3ccccc3cc2-c2c(-c3cc(-c4ccc(-c5ccc(-c6ccc7ncccc7c6)cc5)cc4)nc(-c4ccccc4)n3)cccc21 ; CC1(C)c2cc3ccccc3cc2-c2c(-c3cc(-c4ccc(-c5cccc(-c6ccc7ncccc7c6)c5)cc4)nc(-c4ccccc4)n3)cccc21 ; CC1(C)c2cc3ccccc3cc2-c2c(-c3cc(-c4ccc(-c5ccccc5-c5ccc6ncccc6c5)cc4)nc(-c4ccccc4)n3)cccc21]

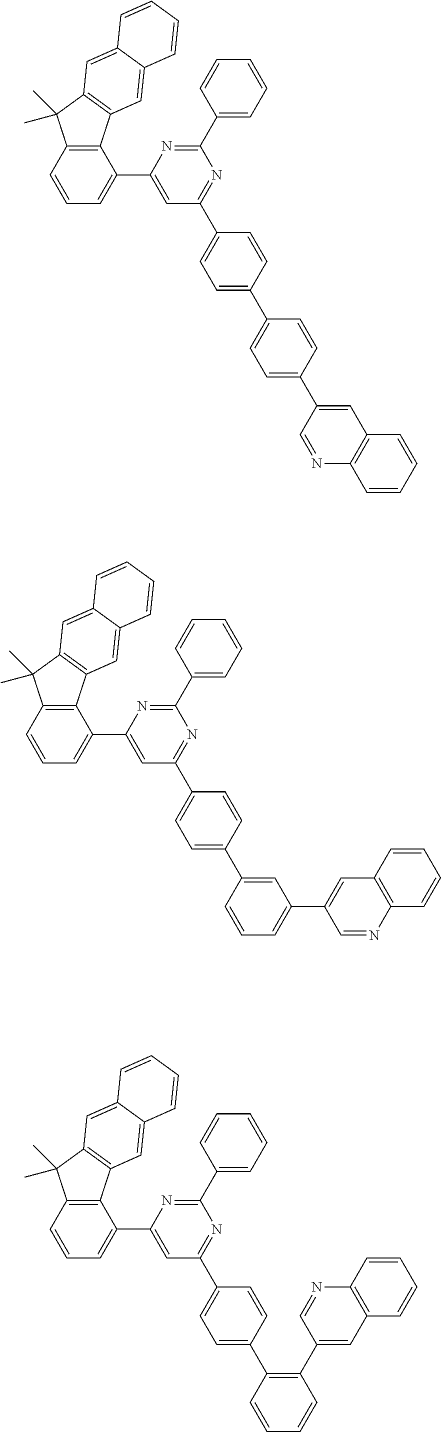 [SMILES: CC1(C)c2cc3ccccc3cc2-c2c(-c3cc(-c4ccc(-c5ccc(-c6cnc7ccccc7c6)cc5)cc4)nc(-c4ccccc4)n3)cccc21 ; CC1(C)c2cc3ccccc3cc2-c2c(-c3cc(-c4ccc(-c5cccc(-c6cnc7ccccc7c6)c5)cc4)nc(-c4ccccc4)n3)cccc21 ; CC1(C)c2cc3ccccc3cc2-c2c(-c3cc(-c4ccc(-c5ccccc5-c5cnc6ccccc6c5)cc4)nc(-c4ccccc4)n3)cccc21]

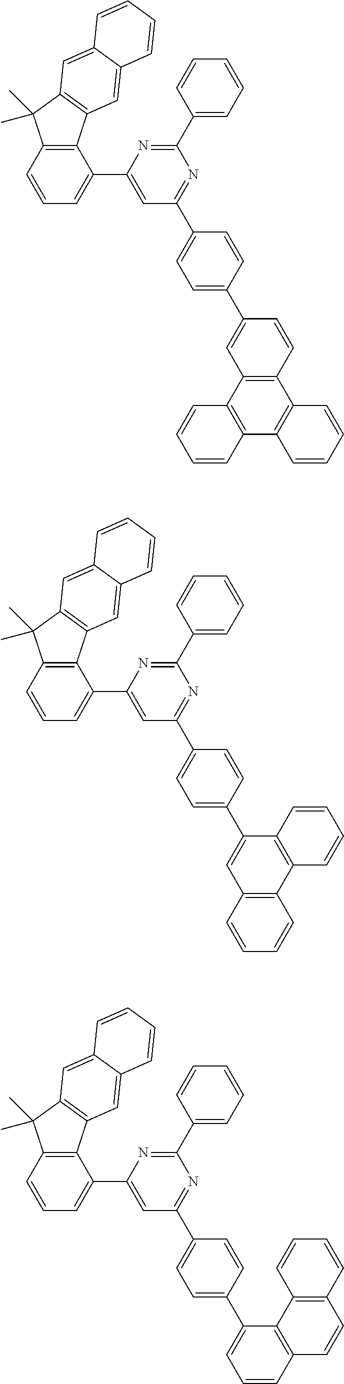 [SMILES: CC1(C)c2cc3ccccc3cc2-c2c(-c3cc(-c4ccc(-c5ccc6c7ccccc7c7ccccc7c6c5)cc4)nc(-c4ccccc4)n3)cccc21 ; CC1(C)c2cc3ccccc3cc2-c2c(-c3cc(-c4ccc(-c5cc6ccccc6c6ccccc56)cc4)nc(-c4ccccc4)n3)cccc21 ; CC1(C)c2cc3ccccc3cc2-c2c(-c3cc(-c4ccc(-c5cccc6ccc7ccccc7c56)cc4)nc(-c4ccccc4)n3)cccc21]

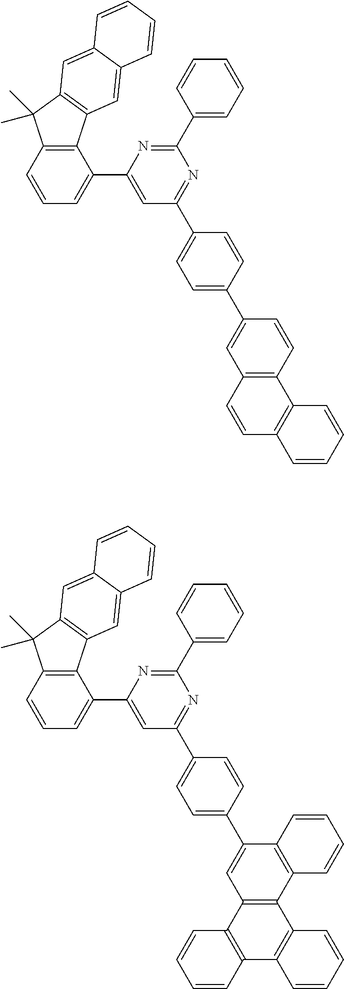 [SMILES: CC1(C)c2cc3ccccc3cc2-c2c(-c3cc(-c4ccc(-c5ccc6c(ccc7ccccc76)c5)cc4)nc(-c4ccccc4)n3)cccc21 ; CC1(C)c2cc3ccccc3cc2-c2c(-c3cc(-c4ccc(-c5cc6c7ccccc7c7ccccc7c6c6ccccc56)cc4)nc(-c4ccccc4)n3)cccc21]

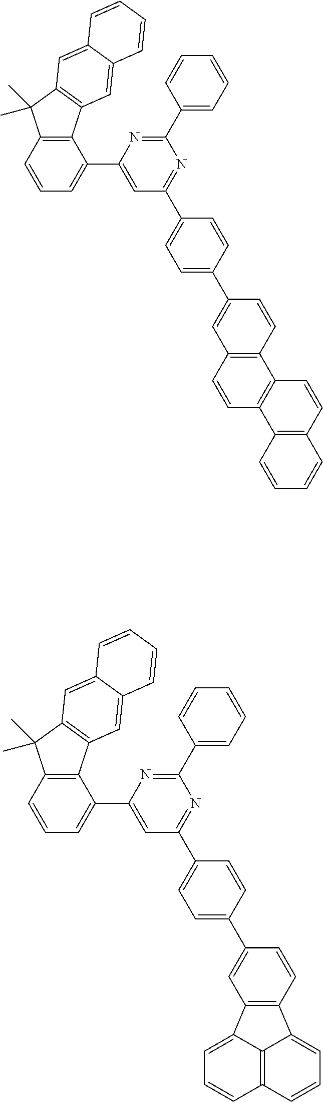 [SMILES: CC1(C)c2cc3ccccc3cc2-c2c(-c3cc(-c4ccc(-c5ccc6c(ccc7c8ccccc8ccc67)c5)cc4)nc(-c4ccccc4)n3)cccc21 ; CC1(C)c2cc3ccccc3cc2-c2c(-c3cc(-c4ccc(-c5ccc6c(c5)-c5cccc7cccc-6c57)cc4)nc(-c4ccccc4)n3)cccc21]

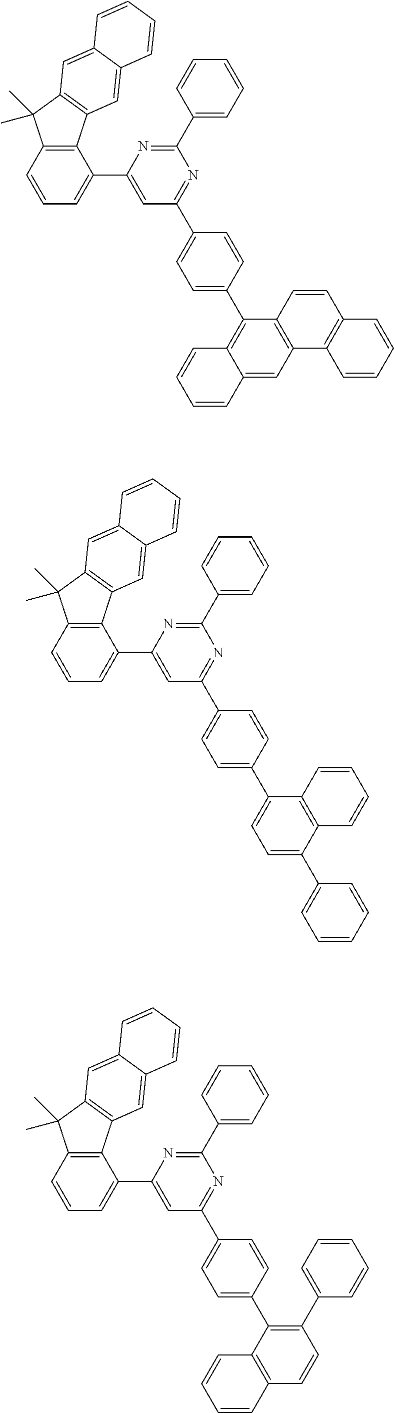 [SMILES: CC1(C)c2cc3ccccc3cc2-c2c(-c3cc(-c4ccc(-c5c6ccccc6cc6c5ccc5ccccc56)cc4)nc(-c4ccccc4)n3)cccc21 ; CC1(C)c2cc3ccccc3cc2-c2c(-c3cc(-c4ccc(-c5ccc(-c6ccccc6)c6ccccc56)cc4)nc(-c4ccccc4)n3)cccc21 ; CC1(C)c2cc3ccccc3cc2-c2c(-c3cc(-c4ccc(-c5c(-c6ccccc6)ccc6ccccc56)cc4)nc(-c4ccccc4)n3)cccc21]

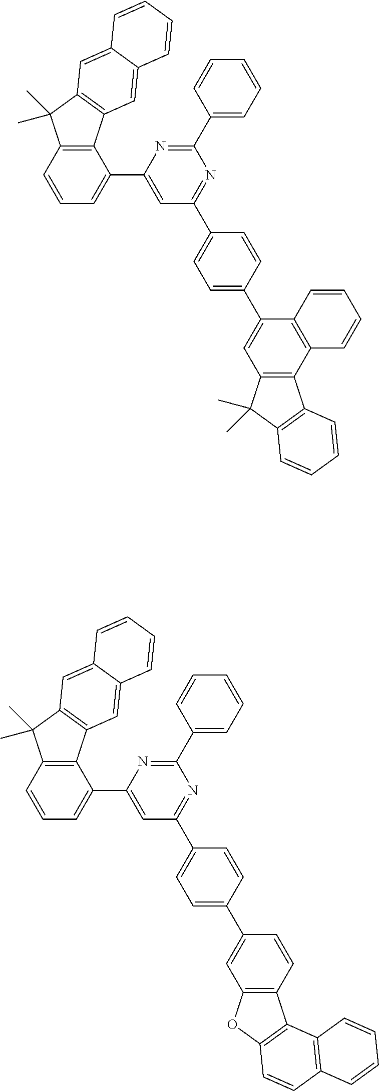 [SMILES: CC1(C)c2cc3ccccc3cc2-c2c(-c3cc(-c4ccc(-c5cc6c(c7ccccc57)-c5ccccc5C6(C)C)cc4)nc(-c4ccccc4)n3)cccc21 ; CC1(C)c2cc3ccccc3cc2-c2c(-c3cc(-c4ccc(-c5ccc6c(c5)oc5ccc7ccccc7c56)cc4)nc(-c4ccccc4)n3)cccc21]

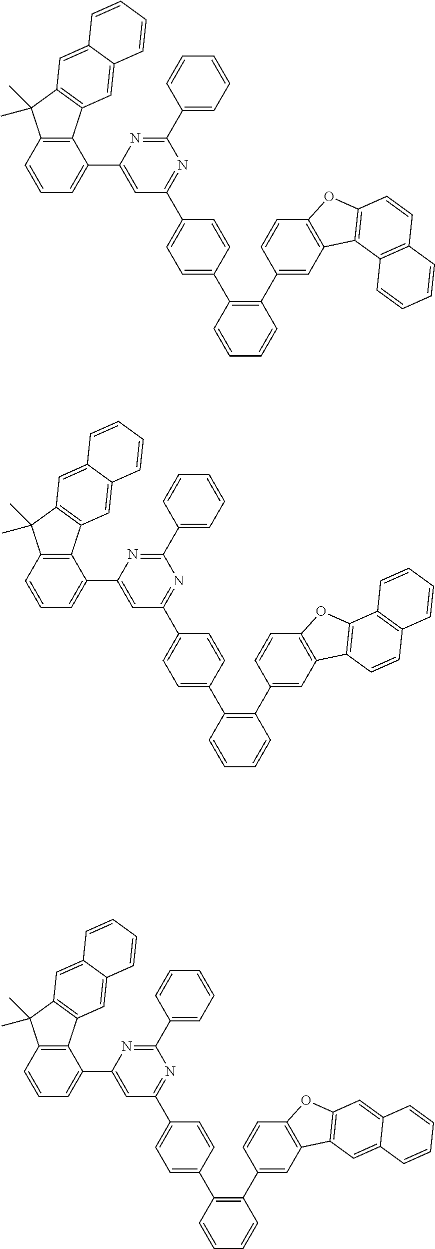 [SMILES: CC1(C)c2cc3ccccc3cc2-c2c(-c3cc(-c4ccc(-c5ccccc5-c5ccc6oc7ccc8ccccc8c7c6c5)cc4)nc(-c4ccccc4)n3)cccc21 ; CC1(C)c2cc3ccccc3cc2-c2c(-c3cc(-c4ccc(-c5ccccc5-c5ccc6oc7c8ccccc8ccc7c6c5)cc4)nc(-c4ccccc4)n3)cccc21 ; CC1(C)c2cc3ccccc3cc2-c2c(-c3cc(-c4ccc(-c5ccccc5-c5ccc6oc7cc8ccccc8cc7c6c5)cc4)nc(-c4ccccc4)n3)cccc21]

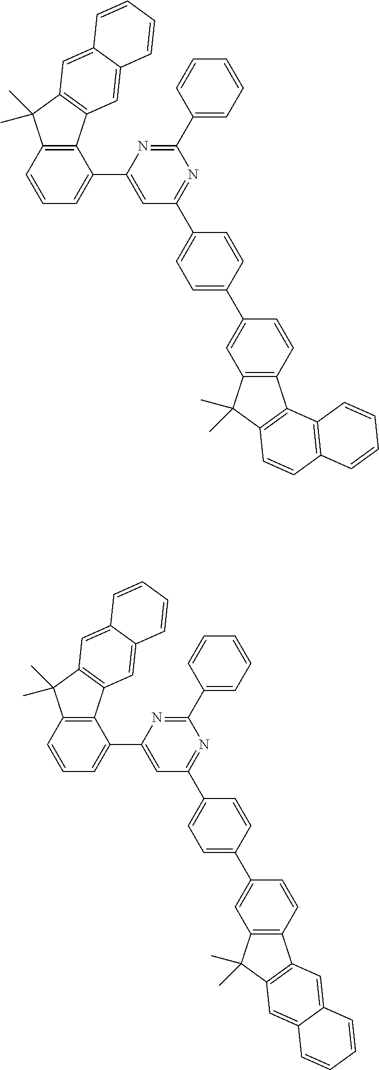 [SMILES: CC1(C)c2cc3ccccc3cc2-c2c(-c3cc(-c4ccc(-c5ccc6c(c5)C(C)(C)c5ccc7ccccc7c5-6)cc4)nc(-c4ccccc4)n3)cccc21 ; CC1(C)c2cc(-c3ccc(-c4cc(-c5cccc6c5-c5cc7ccccc7cc5C6(C)C)nc(-c5ccccc5)n4)cc3)ccc2-c2cc3ccccc3cc21]

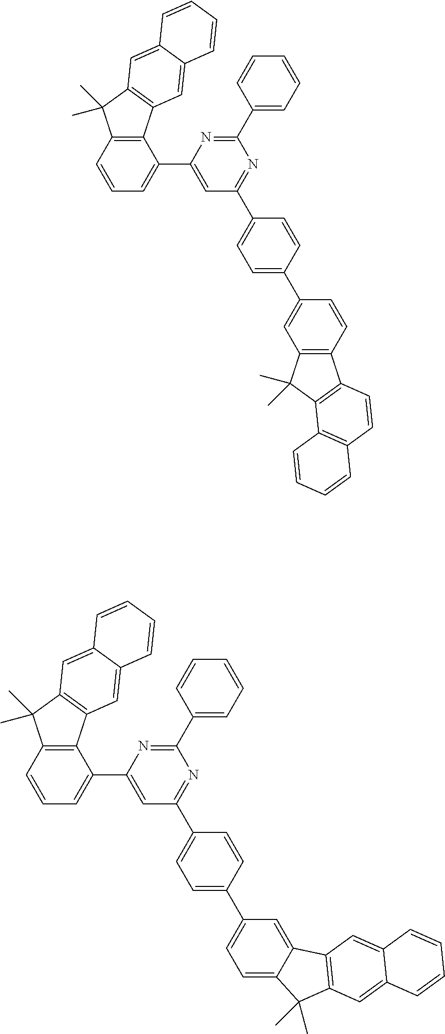 [SMILES: CC1(C)c2cc3ccccc3cc2-c2c(-c3cc(-c4ccc(-c5ccc6c(c5)C(C)(C)c5c-6ccc6ccccc56)cc4)nc(-c4ccccc4)n3)cccc21 ; CC1(C)c2ccc(-c3ccc(-c4cc(-c5cccc6c5-c5cc7ccccc7cc5C6(C)C)nc(-c5ccccc5)n4)cc3)cc2-c2cc3ccccc3cc21]

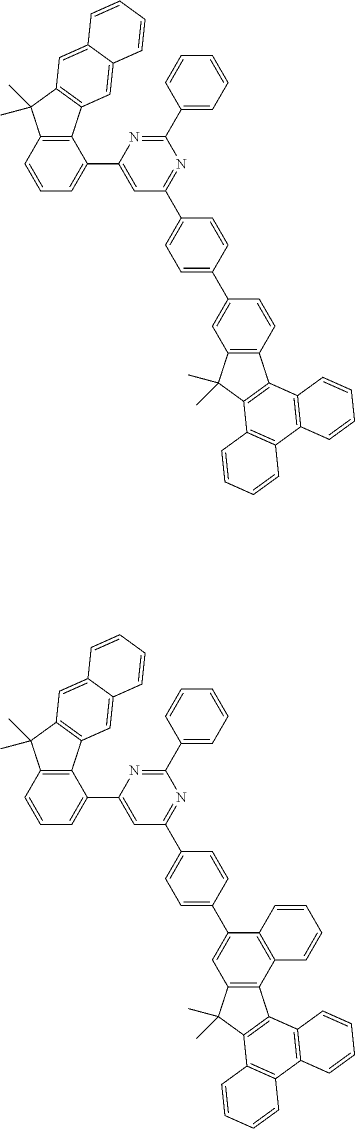 [SMILES: CC1(C)c2cc3ccccc3cc2-c2c(-c3cc(-c4ccc(-c5ccc6c(c5)C(C)(C)c5c-6c6ccccc6c6ccccc56)cc4)nc(-c4ccccc4)n3)cccc21 ; CC1(C)c2cc3ccccc3cc2-c2c(-c3cc(-c4ccc(-c5cc6c(c7ccccc57)-c5c(c7ccccc7c7ccccc57)C6(C)C)cc4)nc(-c4ccccc4)n3)cccc21]

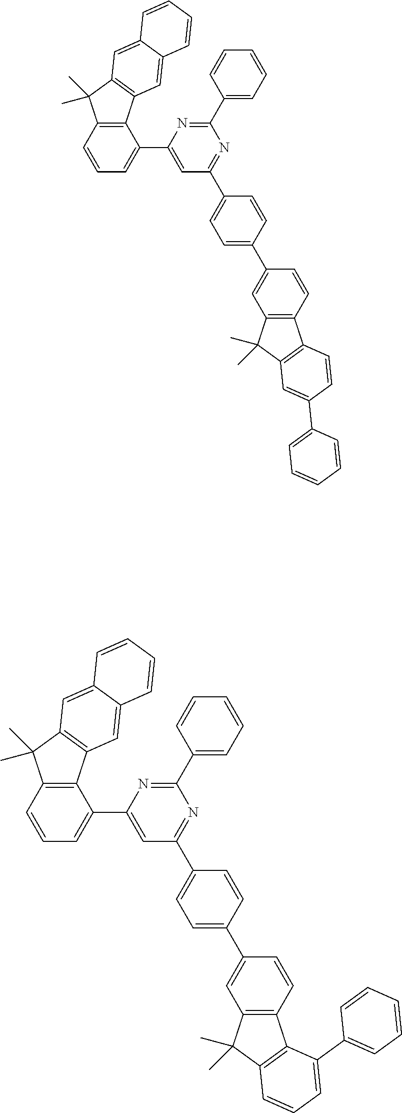 [SMILES: CC1(C)c2cc(-c3ccccc3)ccc2-c2ccc(-c3ccc(-c4cc(-c5cccc6c5-c5cc7ccccc7cc5C6(C)C)nc(-c5ccccc5)n4)cc3)cc21 ; CC1(C)c2cc(-c3ccc(-c4cc(-c5cccc6c5-c5cc7ccccc7cc5C6(C)C)nc(-c5ccccc5)n4)cc3)ccc2-c2c(-c3ccccc3)cccc21]

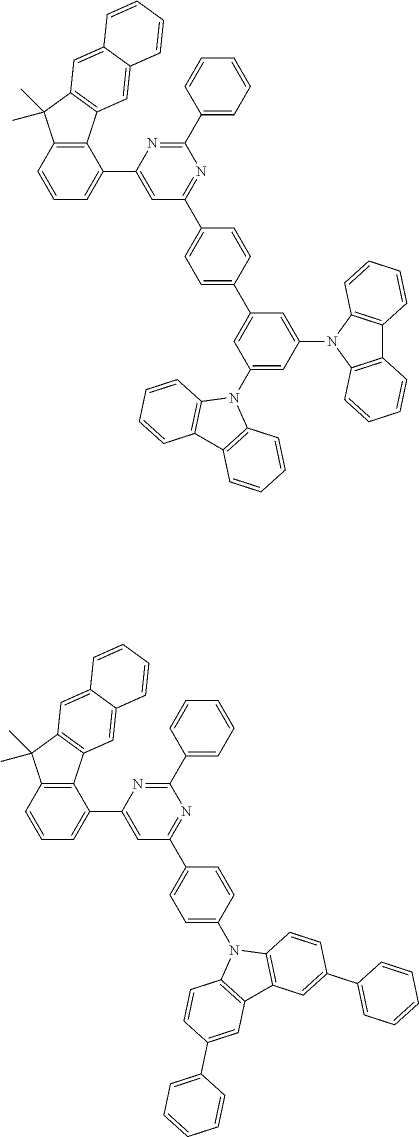 [SMILES: CC1(C)c2cc3ccccc3cc2-c2c(-c3cc(-c4ccc(-c5cc(-n6c7ccccc7c7ccccc76)cc(-n6c7ccccc7c7ccccc76)c5)cc4)nc(-c4ccccc4)n3)cccc21 ; CC1(C)c2cc3ccccc3cc2-c2c(-c3cc(-c4ccc(-n5c6ccc(-c7ccccc7)cc6c6cc(-c7ccccc7)ccc65)cc4)nc(-c4ccccc4)n3)cccc21]

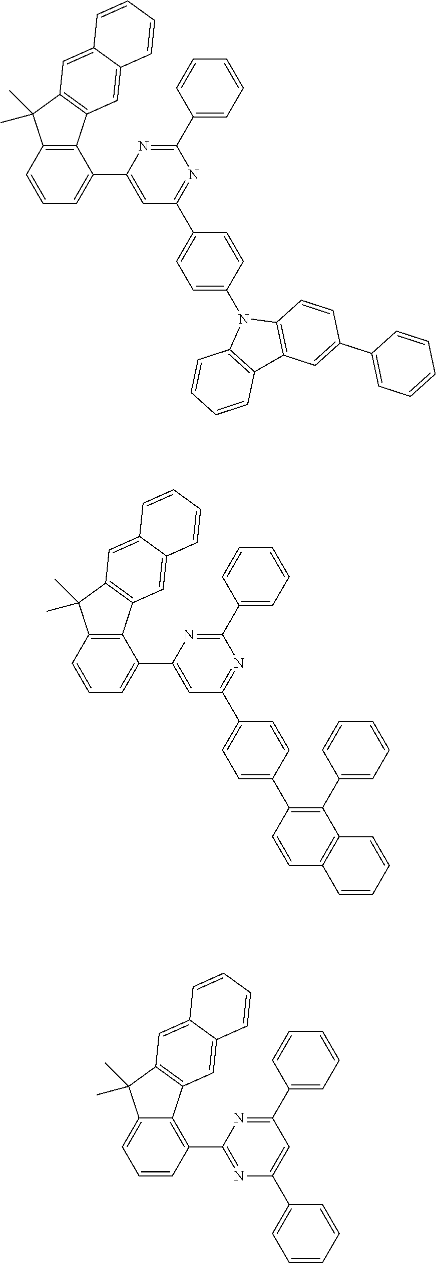 [SMILES: CC1(C)c2cc3ccccc3cc2-c2c(-c3cc(-c4ccc(-n5c6ccccc6c6cc(-c7ccccc7)ccc65)cc4)nc(-c4ccccc4)n3)cccc21 ; CC1(C)c2cc3ccccc3cc2-c2c(-c3cc(-c4ccc(-c5ccc6ccccc6c5-c5ccccc5)cc4)nc(-c4ccccc4)n3)cccc21 ; CC1(C)c2cc3ccccc3cc2-c2c(-c3nc(-c4ccccc4)cc(-c4ccccc4)n3)cccc21]

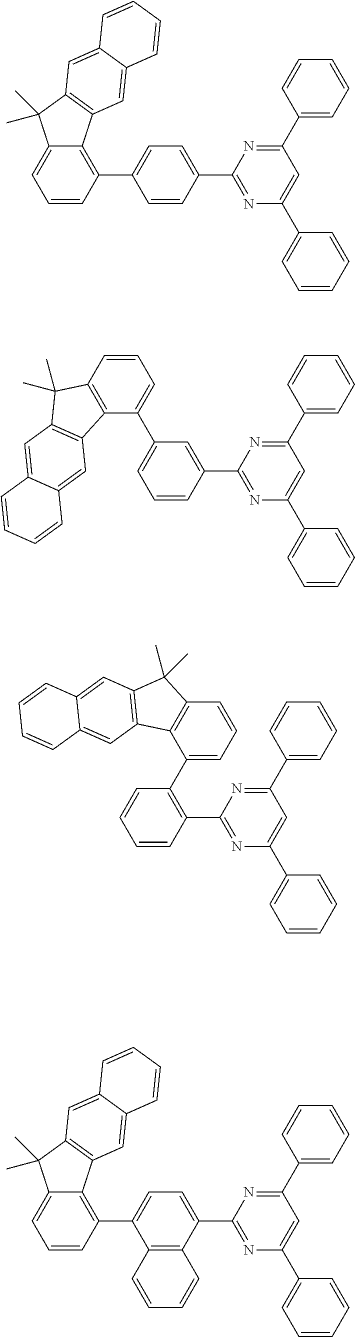 [SMILES: CC1(C)c2cc3ccccc3cc2-c2c(-c3ccc(-c4nc(-c5ccccc5)cc(-c5ccccc5)n4)cc3)cccc21 ; CC1(C)c2cc3ccccc3cc2-c2c(-c3cccc(-c4nc(-c5ccccc5)cc(-c5ccccc5)n4)c3)cccc21 ; CC1(C)c2cc3ccccc3cc2-c2c(-c3ccccc3-c3nc(-c4ccccc4)cc(-c4ccccc4)n3)cccc21 ; CC1(C)c2cc3ccccc3cc2-c2c(-c3ccc(-c4nc(-c5ccccc5)cc(-c5ccccc5)n4)c4ccccc34)cccc21]

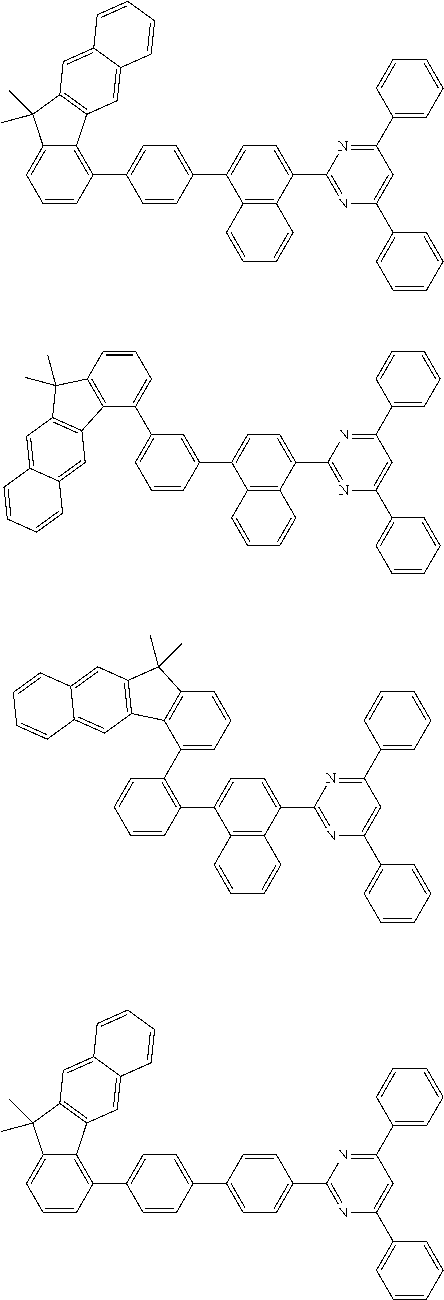 [SMILES: CC1(C)c2cc3ccccc3cc2-c2c(-c3ccc(-c4ccc(-c5nc(-c6ccccc6)cc(-c6ccccc6)n5)c5ccccc45)cc3)cccc21 ; CC1(C)c2cc3ccccc3cc2-c2c(-c3cccc(-c4ccc(-c5nc(-c6ccccc6)cc(-c6ccccc6)n5)c5ccccc45)c3)cccc21 ; CC1(C)c2cc3ccccc3cc2-c2c(-c3ccccc3-c3ccc(-c4nc(-c5ccccc5)cc(-c5ccccc5)n4)c4ccccc34)cccc21 ; CC1(C)c2cc3ccccc3cc2-c2c(-c3ccc(-c4ccc(-c5nc(-c6ccccc6)cc(-c6ccccc6)n5)cc4)cc3)cccc21]

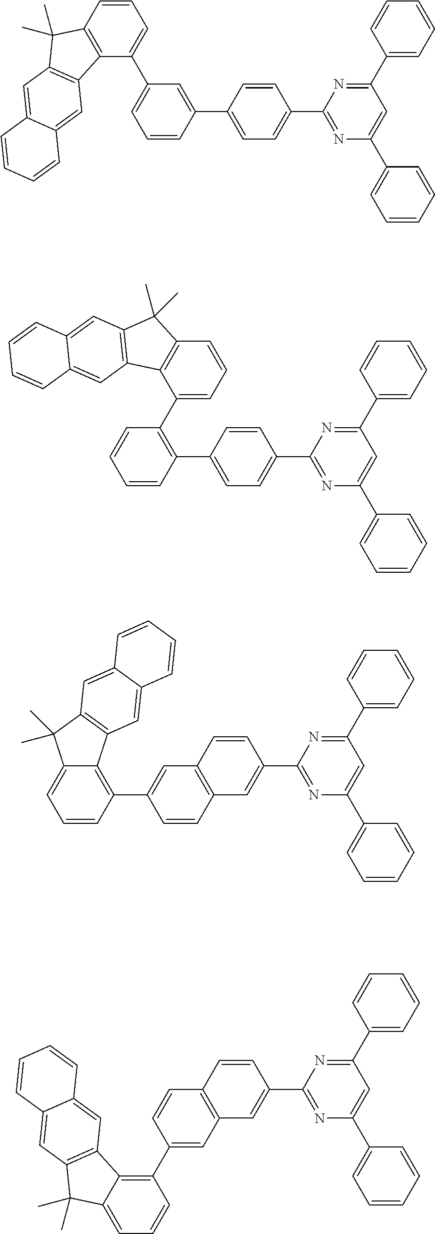 [SMILES: CC1(C)c2cc3ccccc3cc2-c2c(-c3cccc(-c4ccc(-c5nc(-c6ccccc6)cc(-c6ccccc6)n5)cc4)c3)cccc21 ; CC1(C)c2cc3ccccc3cc2-c2c(-c3ccccc3-c3ccc(-c4nc(-c5ccccc5)cc(-c5ccccc5)n4)cc3)cccc21 ; CC1(C)c2cc3ccccc3cc2-c2c(-c3ccc4cc(-c5nc(-c6ccccc6)cc(-c6ccccc6)n5)ccc4c3)cccc21 ; CC1(C)c2cc3ccccc3cc2-c2c(-c3ccc4ccc(-c5nc(-c6ccccc6)cc(-c6ccccc6)n5)cc4c3)cccc21]

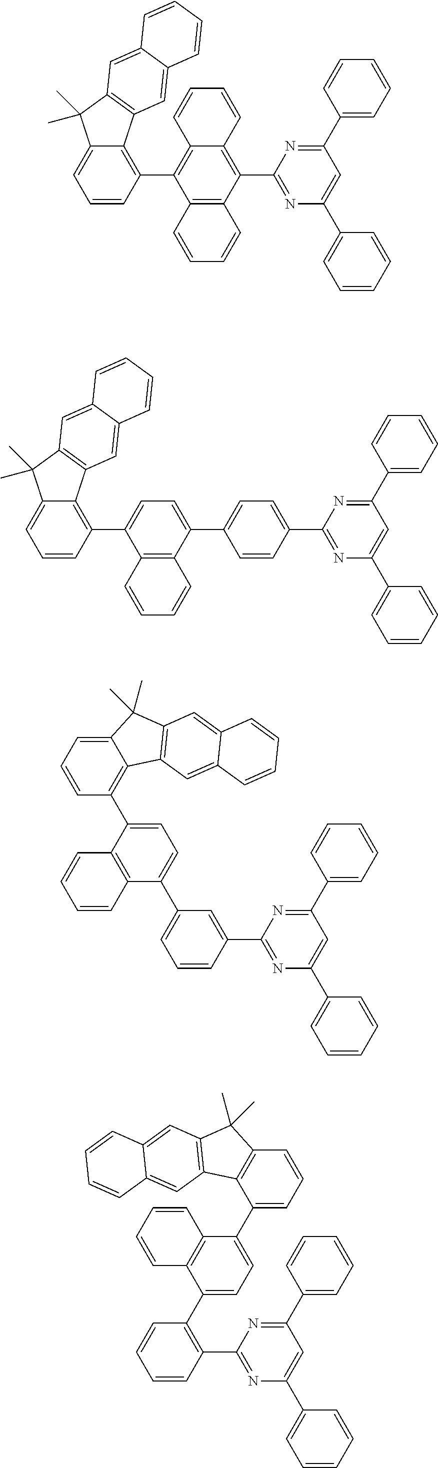 [SMILES: CC1(C)c2cc3ccccc3cc2-c2c(-c3c4ccccc4c(-c4nc(-c5ccccc5)cc(-c5ccccc5)n4)c4ccccc34)cccc21 ; CC1(C)c2cc3ccccc3cc2-c2c(-c3ccc(-c4ccc(-c5nc(-c6ccccc6)cc(-c6ccccc6)n5)cc4)c4ccccc34)cccc21 ; CC1(C)c2cc3ccccc3cc2-c2c(-c3ccc(-c4cccc(-c5nc(-c6ccccc6)cc(-c6ccccc6)n5)c4)c4ccccc34)cccc21 ; CC1(C)c2cc3ccccc3cc2-c2c(-c3ccc(-c4ccccc4-c4nc(-c5ccccc5)cc(-c5ccccc5)n4)c4ccccc34)cccc21]

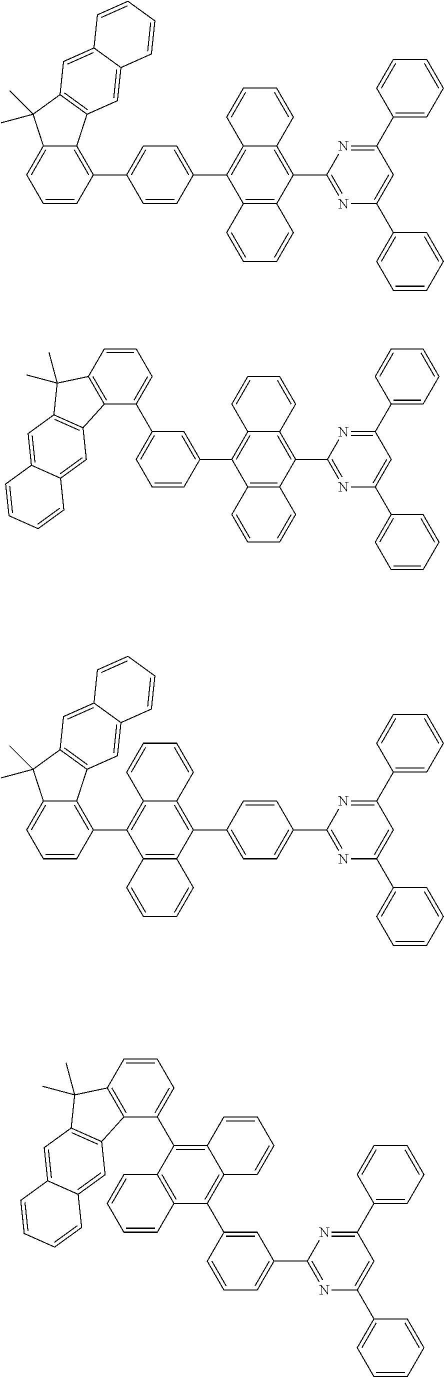 [SMILES: CC1(C)c2cc3ccccc3cc2-c2c(-c3ccc(-c4c5ccccc5c(-c5nc(-c6ccccc6)cc(-c6ccccc6)n5)c5ccccc45)cc3)cccc21 ; CC1(C)c2cc3ccccc3cc2-c2c(-c3cccc(-c4c5ccccc5c(-c5nc(-c6ccccc6)cc(-c6ccccc6)n5)c5ccccc45)c3)cccc21 ; CC1(C)c2cc3ccccc3cc2-c2c(-c3c4ccccc4c(-c4ccc(-c5nc(-c6ccccc6)cc(-c6ccccc6)n5)cc4)c4ccccc34)cccc21 ; CC1(C)c2cc3ccccc3cc2-c2c(-c3c4ccccc4c(-c4cccc(-c5nc(-c6ccccc6)cc(-c6ccccc6)n5)c4)c4ccccc34)cccc21]

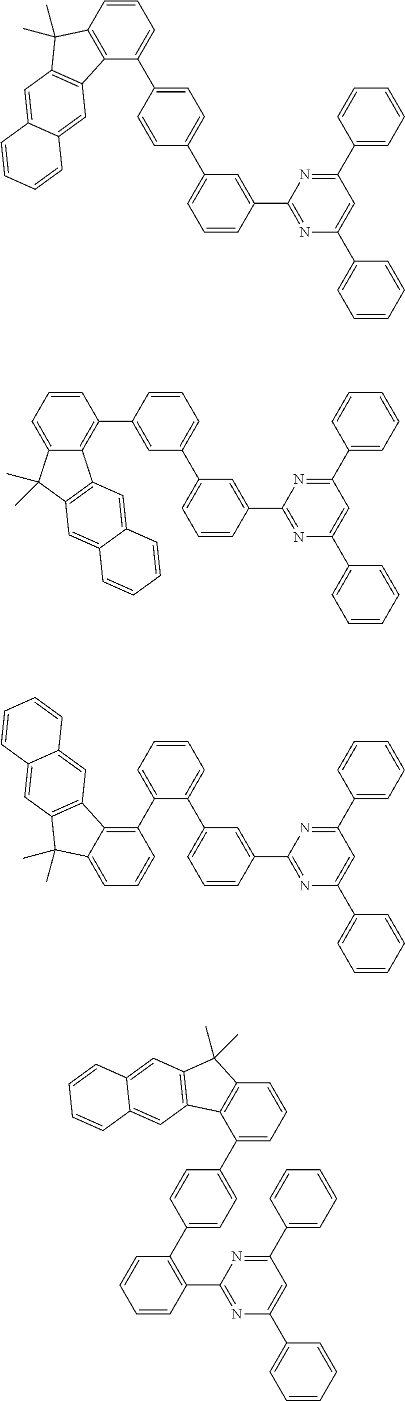 [SMILES: CC1(C)c2cc3ccccc3cc2-c2c(-c3ccc(-c4cccc(-c5nc(-c6ccccc6)cc(-c6ccccc6)n5)c4)cc3)cccc21 ; CC1(C)c2cc3ccccc3cc2-c2c(-c3cccc(-c4cccc(-c5nc(-c6ccccc6)cc(-c6ccccc6)n5)c4)c3)cccc21 ; CC1(C)c2cc3ccccc3cc2-c2c(-c3ccccc3-c3cccc(-c4nc(-c5ccccc5)cc(-c5ccccc5)n4)c3)cccc21 ; CC1(C)c2cc3ccccc3cc2-c2c(-c3ccc(-c4ccccc4-c4nc(-c5ccccc5)cc(-c5ccccc5)n4)cc3)cccc21]

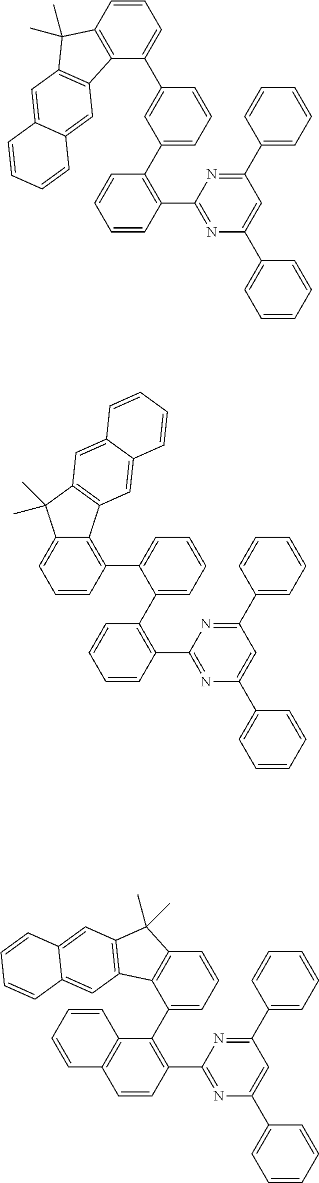 [SMILES: CC1(C)c2cc3ccccc3cc2-c2c(-c3cccc(-c4ccccc4-c4nc(-c5ccccc5)cc(-c5ccccc5)n4)c3)cccc21 ; CC1(C)c2cc3ccccc3cc2-c2c(-c3ccccc3-c3ccccc3-c3nc(-c4ccccc4)cc(-c4ccccc4)n3)cccc21 ; CC1(C)c2cc3ccccc3cc2-c2c(-c3c(-c4nc(-c5ccccc5)cc(-c5ccccc5)n4)ccc4ccccc34)cccc21]

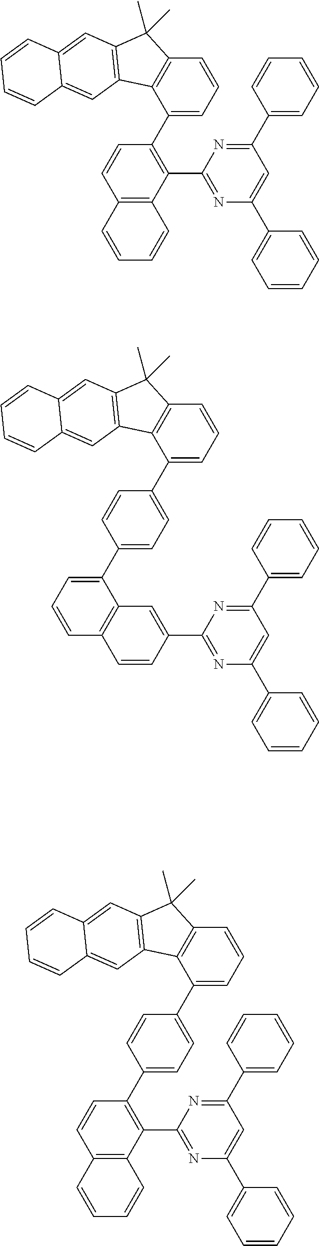 [SMILES: CC1(C)c2cc3ccccc3cc2-c2c(-c3ccc4ccccc4c3-c3nc(-c4ccccc4)cc(-c4ccccc4)n3)cccc21 ; CC1(C)c2cc3ccccc3cc2-c2c(-c3ccc(-c4cccc5ccc(-c6nc(-c7ccccc7)cc(-c7ccccc7)n6)cc45)cc3)cccc21 ; CC1(C)c2cc3ccccc3cc2-c2c(-c3ccc(-c4ccc5ccccc5c4-c4nc(-c5ccccc5)cc(-c5ccccc5)n4)cc3)cccc21]

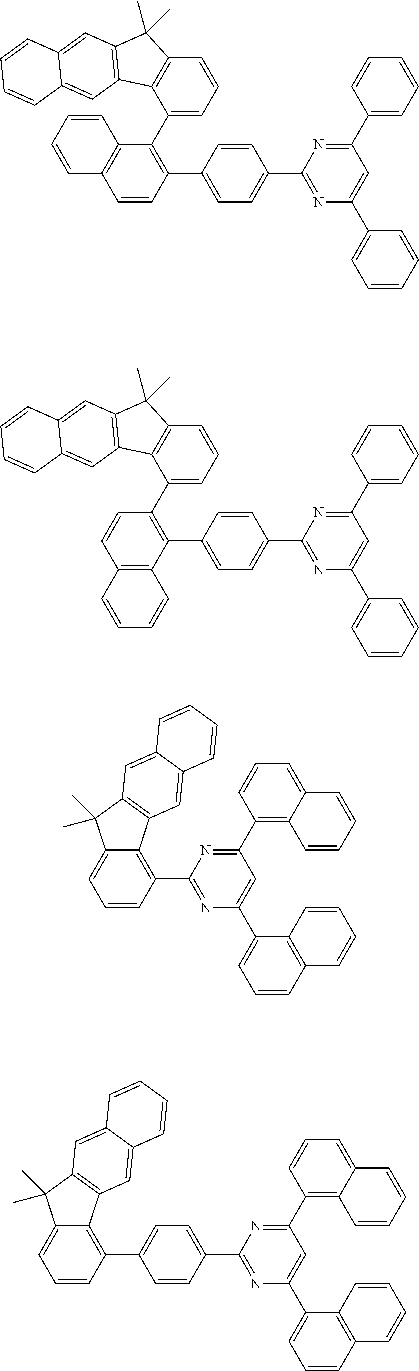 [SMILES: CC1(C)c2cc3ccccc3cc2-c2c(-c3c(-c4ccc(-c5nc(-c6ccccc6)cc(-c6ccccc6)n5)cc4)ccc4ccccc34)cccc21 ; CC1(C)c2cc3ccccc3cc2-c2c(-c3ccc4ccccc4c3-c3ccc(-c4nc(-c5ccccc5)cc(-c5ccccc5)n4)cc3)cccc21 ; CC1(C)c2cc3ccccc3cc2-c2c(-c3nc(-c4cccc5ccccc45)cc(-c4cccc5ccccc45)n3)cccc21 ; CC1(C)c2cc3ccccc3cc2-c2c(-c3ccc(-c4nc(-c5cccc6ccccc56)cc(-c5cccc6ccccc56)n4)cc3)cccc21]

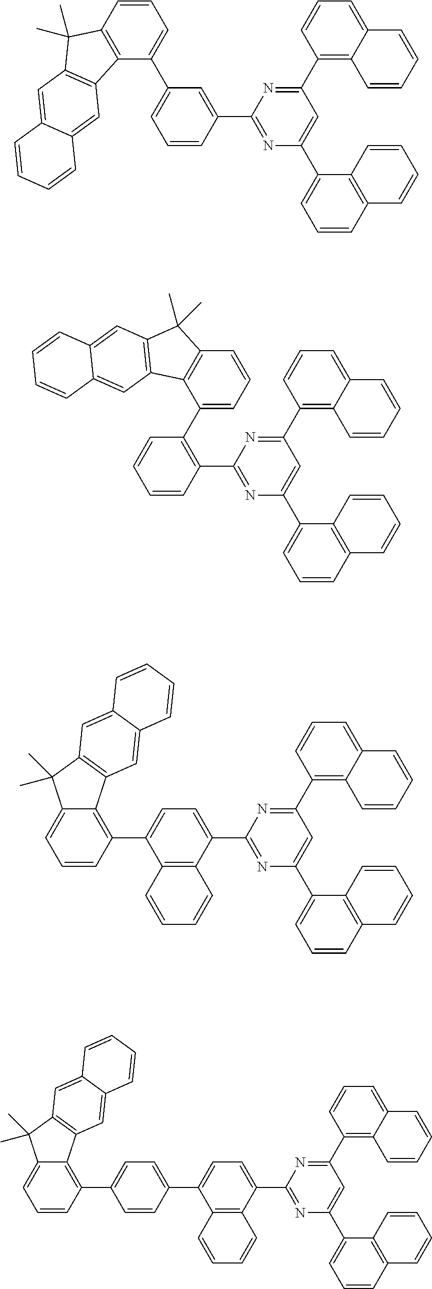 [SMILES: CC1(C)c2cc3ccccc3cc2-c2c(-c3cccc(-c4nc(-c5cccc6ccccc56)cc(-c5cccc6ccccc56)n4)c3)cccc21 ; CC1(C)c2cc3ccccc3cc2-c2c(-c3ccccc3-c3nc(-c4cccc5ccccc45)cc(-c4cccc5ccccc45)n3)cccc21 ; CC1(C)c2cc3ccccc3cc2-c2c(-c3ccc(-c4nc(-c5cccc6ccccc56)cc(-c5cccc6ccccc56)n4)c4ccccc34)cccc21 ; CC1(C)c2cc3ccccc3cc2-c2c(-c3ccc(-c4ccc(-c5nc(-c6cccc7ccccc67)cc(-c6cccc7ccccc67)n5)c5ccccc45)cc3)cccc21]

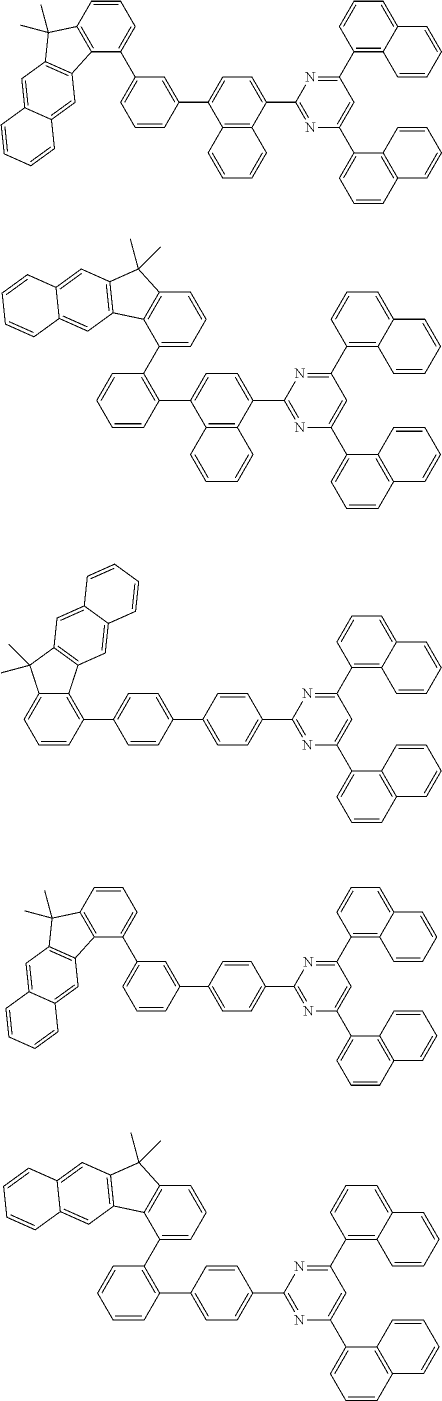

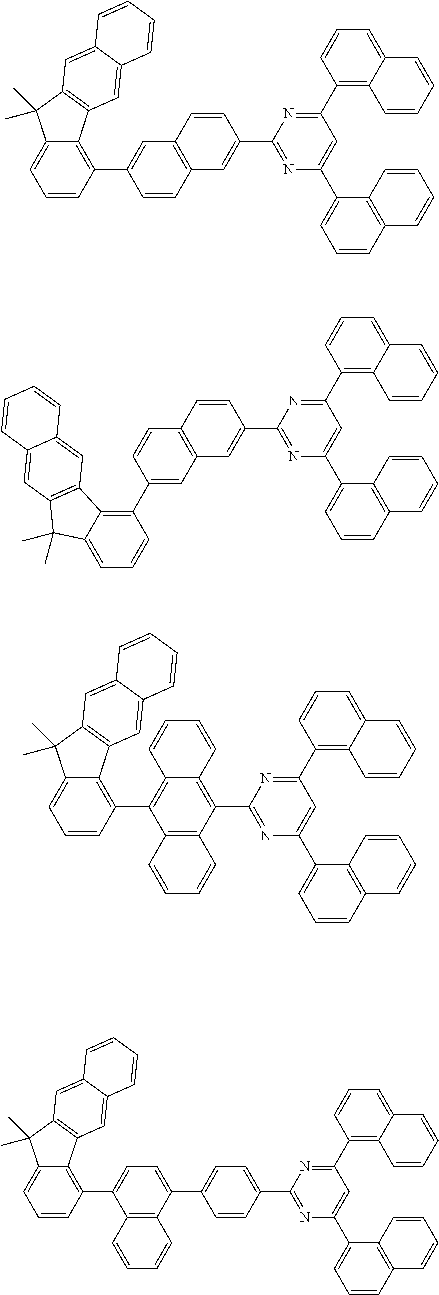 [SMILES: CC1(C)c2cc3ccccc3cc2-c2c(-c3ccc4cc(-c5nc(-c6cccc7ccccc67)cc(-c6cccc7ccccc67)n5)ccc4c3)cccc21 ; CC1(C)c2cc3ccccc3cc2-c2c(-c3ccc4ccc(-c5nc(-c6cccc7ccccc67)cc(-c6cccc7ccccc67)n5)cc4c3)cccc21 ; CC1(C)c2cc3ccccc3cc2-c2c(-c3c4ccccc4c(-c4nc(-c5cccc6ccccc56)cc(-c5cccc6ccccc56)n4)c4ccccc34)cccc21 ; CC1(C)c2cc3ccccc3cc2-c2c(-c3ccc(-c4ccc(-c5nc(-c6cccc7ccccc67)cc(-c6cccc7ccccc67)n5)cc4)c4ccccc34)cccc21]

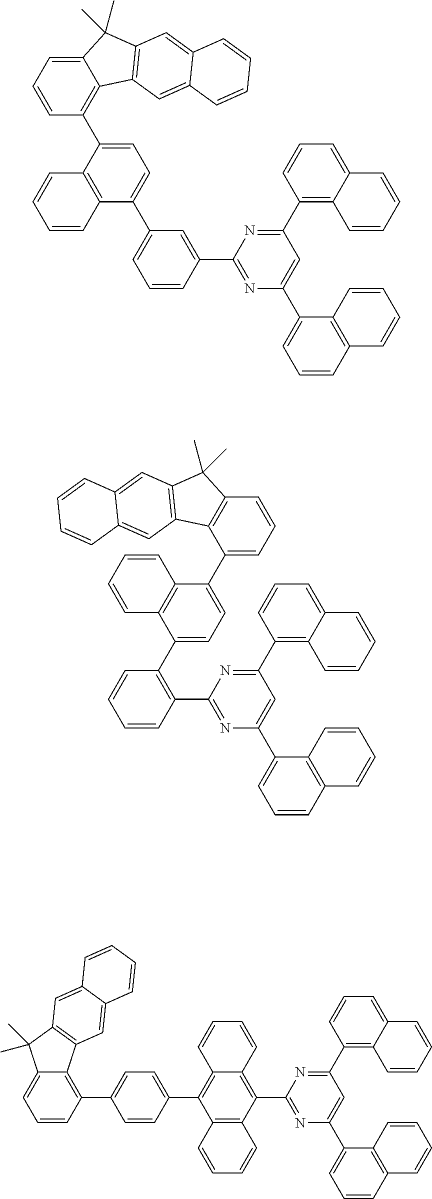 [SMILES: CC1(C)c2cc3ccccc3cc2-c2c(-c3ccc(-c4cccc(-c5nc(-c6cccc7ccccc67)cc(-c6cccc7ccccc67)n5)c4)c4ccccc34)cccc21 ; CC1(C)c2cc3ccccc3cc2-c2c(-c3ccc(-c4ccccc4-c4nc(-c5cccc6ccccc56)cc(-c5cccc6ccccc56)n4)c4ccccc34)cccc21 ; CC1(C)c2cc3ccccc3cc2-c2c(-c3ccc(-c4c5ccccc5c(-c5nc(-c6cccc7ccccc67)cc(-c6cccc7ccccc67)n5)c5ccccc45)cc3)cccc21]

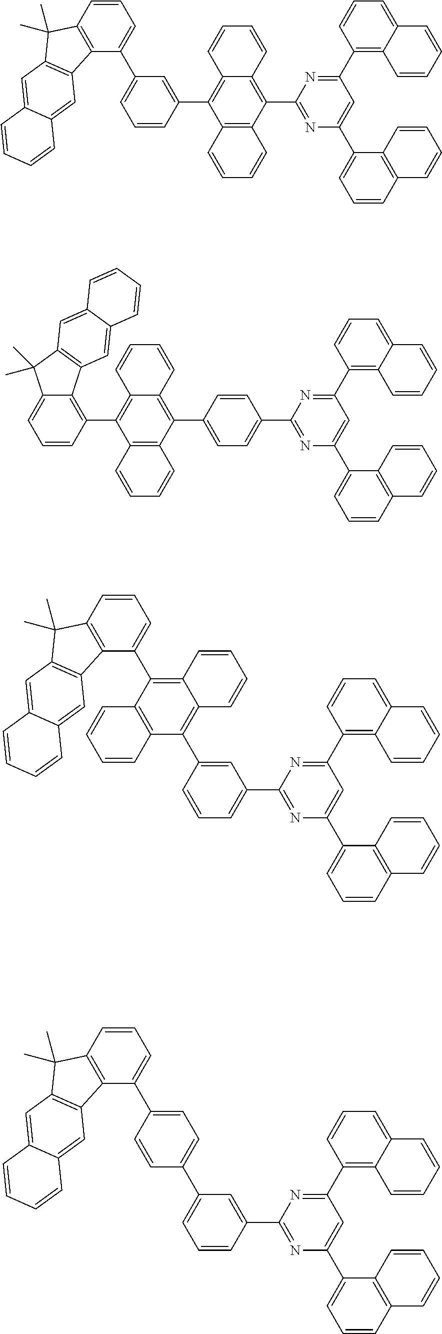 [SMILES: CC1(C)c2cc3ccccc3cc2-c2c(-c3cccc(-c4c5ccccc5c(-c5nc(-c6cccc7ccccc67)cc(-c6cccc7ccccc67)n5)c5ccccc45)c3)cccc21 ; CC1(C)c2cc3ccccc3cc2-c2c(-c3c4ccccc4c(-c4ccc(-c5nc(-c6cccc7ccccc67)cc(-c6cccc7ccccc67)n5)cc4)c4ccccc34)cccc21 ; CC1(C)c2cc3ccccc3cc2-c2c(-c3c4ccccc4c(-c4cccc(-c5nc(-c6cccc7ccccc67)cc(-c6cccc7ccccc67)n5)c4)c4ccccc34)cccc21 ; CC1(C)c2cc3ccccc3cc2-c2c(-c3ccc(-c4cccc(-c5nc(-c6cccc7ccccc67)cc(-c6cccc7ccccc67)n5)c4)cc3)cccc21]

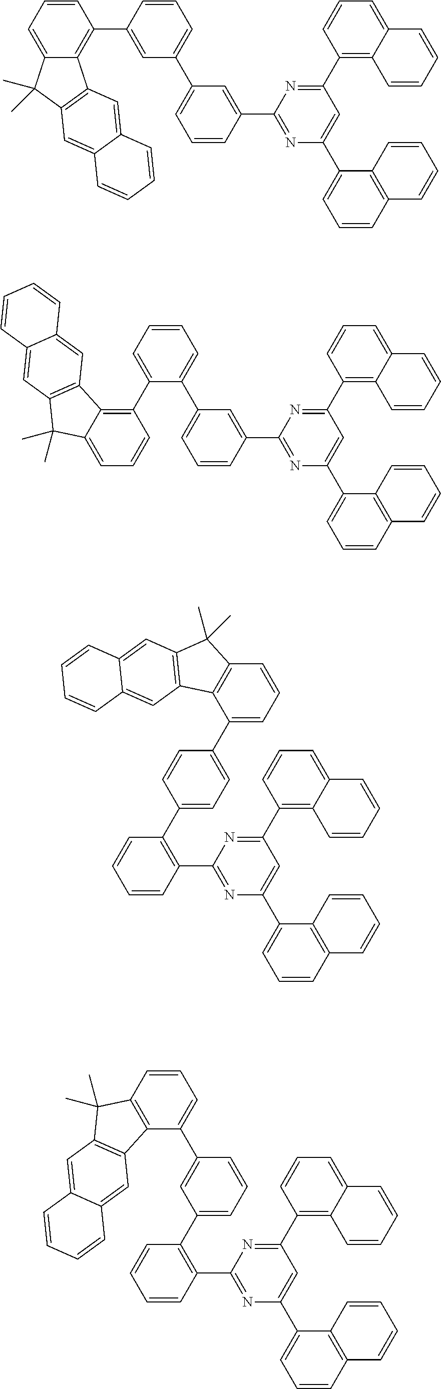 [SMILES: CC1(C)c2cc3ccccc3cc2-c2c(-c3cccc(-c4cccc(-c5nc(-c6cccc7ccccc67)cc(-c6cccc7ccccc67)n5)c4)c3)cccc21 ; CC1(C)c2cc3ccccc3cc2-c2c(-c3ccccc3-c3cccc(-c4nc(-c5cccc6ccccc56)cc(-c5cccc6ccccc56)n4)c3)cccc21 ; CC1(C)c2cc3ccccc3cc2-c2c(-c3ccc(-c4ccccc4-c4nc(-c5cccc6ccccc56)cc(-c5cccc6ccccc56)n4)cc3)cccc21 ; CC1(C)c2cc3ccccc3cc2-c2c(-c3cccc(-c4ccccc4-c4nc(-c5cccc6ccccc56)cc(-c5cccc6ccccc56)n4)c3)cccc21]

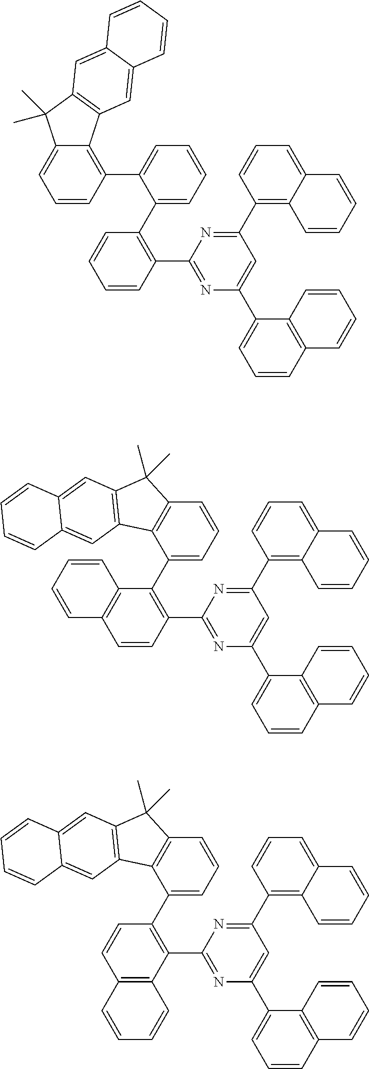 [SMILES: CC1(C)c2cc3ccccc3cc2-c2c(-c3ccccc3-c3ccccc3-c3nc(-c4cccc5ccccc45)cc(-c4cccc5ccccc45)n3)cccc21 ; CC1(C)c2cc3ccccc3cc2-c2c(-c3c(-c4nc(-c5cccc6ccccc56)cc(-c5cccc6ccccc56)n4)ccc4ccccc34)cccc21 ; CC1(C)c2cc3ccccc3cc2-c2c(-c3ccc4ccccc4c3-c3nc(-c4cccc5ccccc45)cc(-c4cccc5ccccc45)n3)cccc21]

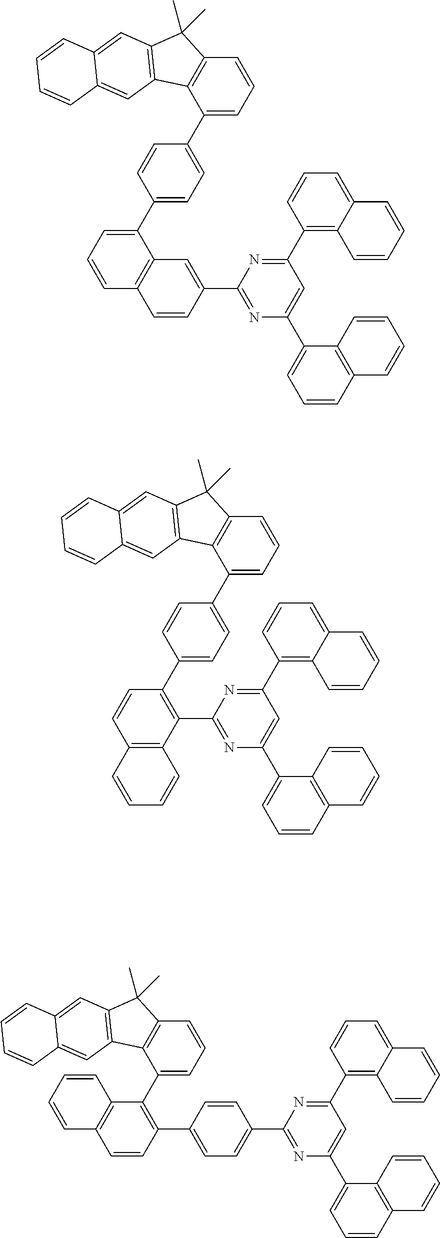 [SMILES: CC1(C)c2cc3ccccc3cc2-c2c(-c3ccc(-c4cccc5ccc(-c6nc(-c7cccc8ccccc78)cc(-c7cccc8ccccc78)n6)cc45)cc3)cccc21 ; CC1(C)c2cc3ccccc3cc2-c2c(-c3ccc(-c4ccc5ccccc5c4-c4nc(-c5cccc6ccccc56)cc(-c5cccc6ccccc56)n4)cc3)cccc21 ; CC1(C)c2cc3ccccc3cc2-c2c(-c3c(-c4ccc(-c5nc(-c6cccc7ccccc67)cc(-c6cccc7ccccc67)n5)cc4)ccc4ccccc34)cccc21]

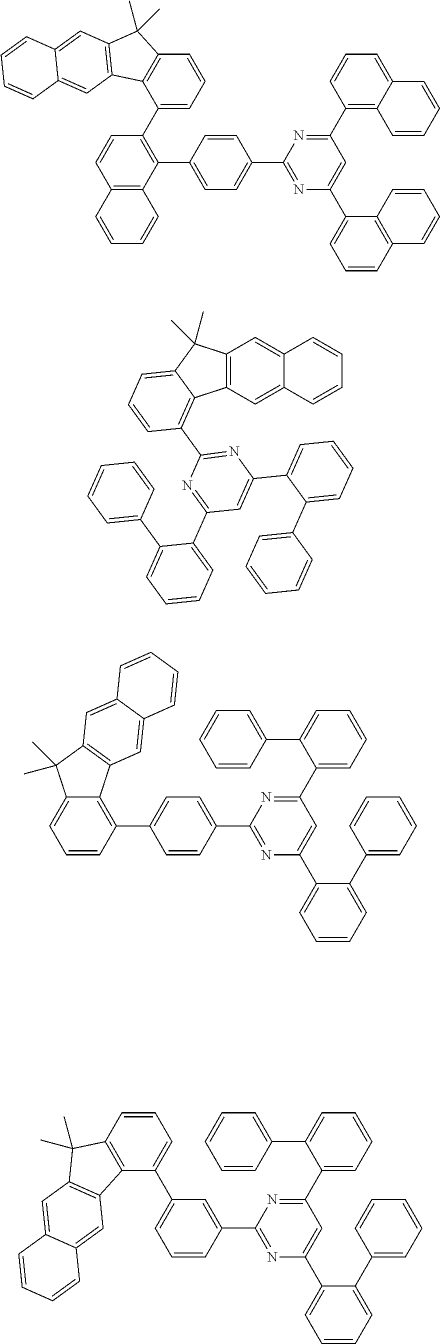 [SMILES: CC1(C)c2cc3ccccc3cc2-c2c(-c3ccc4ccccc4c3-c3ccc(-c4nc(-c5cccc6ccccc56)cc(-c5cccc6ccccc56)n4)cc3)cccc21 ; CC1(C)c2cc3ccccc3cc2-c2c(-c3nc(-c4ccccc4-c4ccccc4)cc(-c4ccccc4-c4ccccc4)n3)cccc21 ; CC1(C)c2cc3ccccc3cc2-c2c(-c3ccc(-c4nc(-c5ccccc5-c5ccccc5)cc(-c5ccccc5-c5ccccc5)n4)cc3)cccc21 ; CC1(C)c2cc3ccccc3cc2-c2c(-c3cccc(-c4nc(-c5ccccc5-c5ccccc5)cc(-c5ccccc5-c5ccccc5)n4)c3)cccc21]

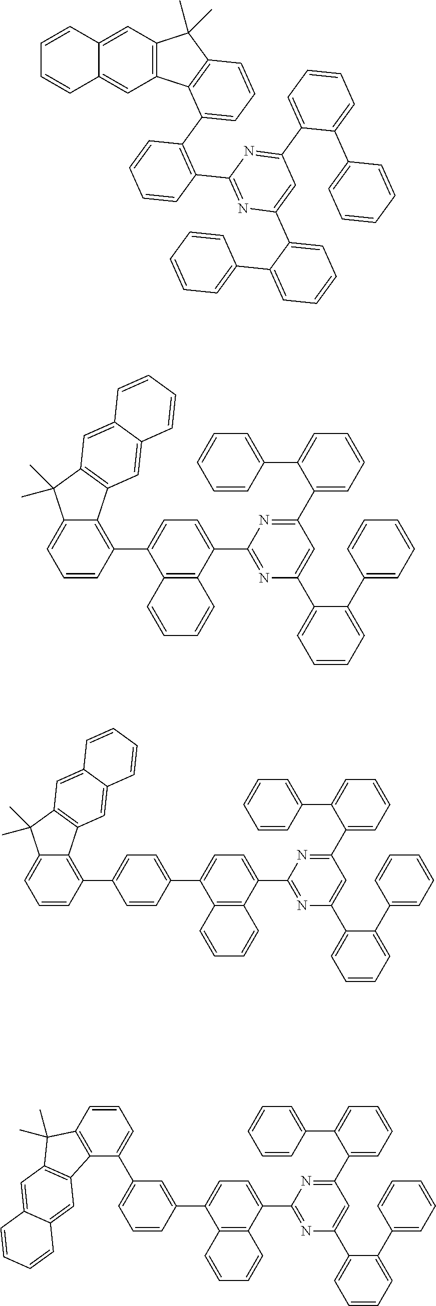 [SMILES: CC1(C)c2cc3ccccc3cc2-c2c(-c3ccccc3-c3nc(-c4ccccc4-c4ccccc4)cc(-c4ccccc4-c4ccccc4)n3)cccc21 ; CC1(C)c2cc3ccccc3cc2-c2c(-c3ccc(-c4nc(-c5ccccc5-c5ccccc5)cc(-c5ccccc5-c5ccccc5)n4)c4ccccc34)cccc21 ; CC1(C)c2cc3ccccc3cc2-c2c(-c3ccc(-c4ccc(-c5nc(-c6ccccc6-c6ccccc6)cc(-c6ccccc6-c6ccccc6)n5)c5ccccc45)cc3)cccc21 ; CC1(C)c2cc3ccccc3cc2-c2c(-c3cccc(-c4ccc(-c5nc(-c6ccccc6-c6ccccc6)cc(-c6ccccc6-c6ccccc6)n5)c5ccccc45)c3)cccc21]

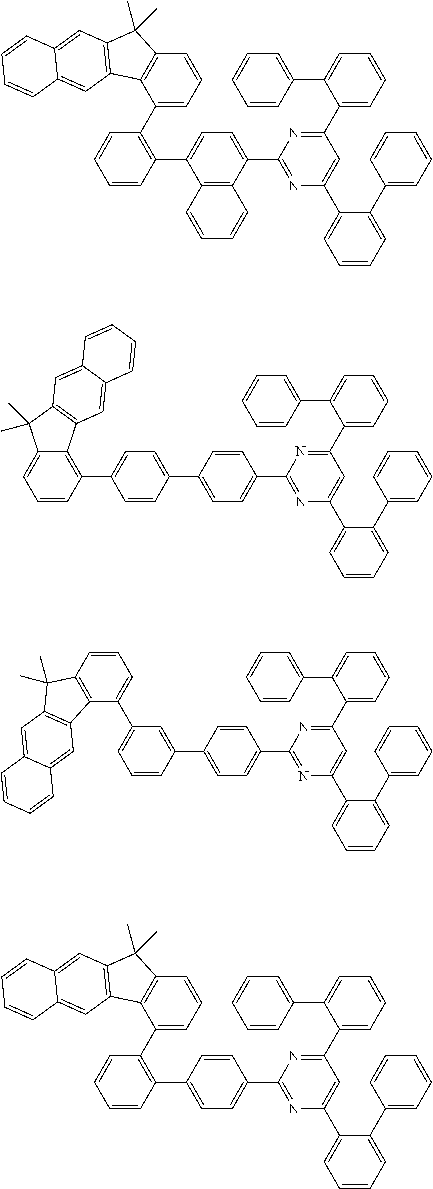 [SMILES: CC1(C)c2cc3ccccc3cc2-c2c(-c3ccccc3-c3ccc(-c4nc(-c5ccccc5-c5ccccc5)cc(-c5ccccc5-c5ccccc5)n4)c4ccccc34)cccc21 ; CC1(C)c2cc3ccccc3cc2-c2c(-c3ccc(-c4ccc(-c5nc(-c6ccccc6-c6ccccc6)cc(-c6ccccc6-c6ccccc6)n5)cc4)cc3)cccc21 ; CC1(C)c2cc3ccccc3cc2-c2c(-c3cccc(-c4ccc(-c5nc(-c6ccccc6-c6ccccc6)cc(-c6ccccc6-c6ccccc6)n5)cc4)c3)cccc21 ; CC1(C)c2cc3ccccc3cc2-c2c(-c3ccccc3-c3ccc(-c4nc(-c5ccccc5-c5ccccc5)cc(-c5ccccc5-c5ccccc5)n4)cc3)cccc21]

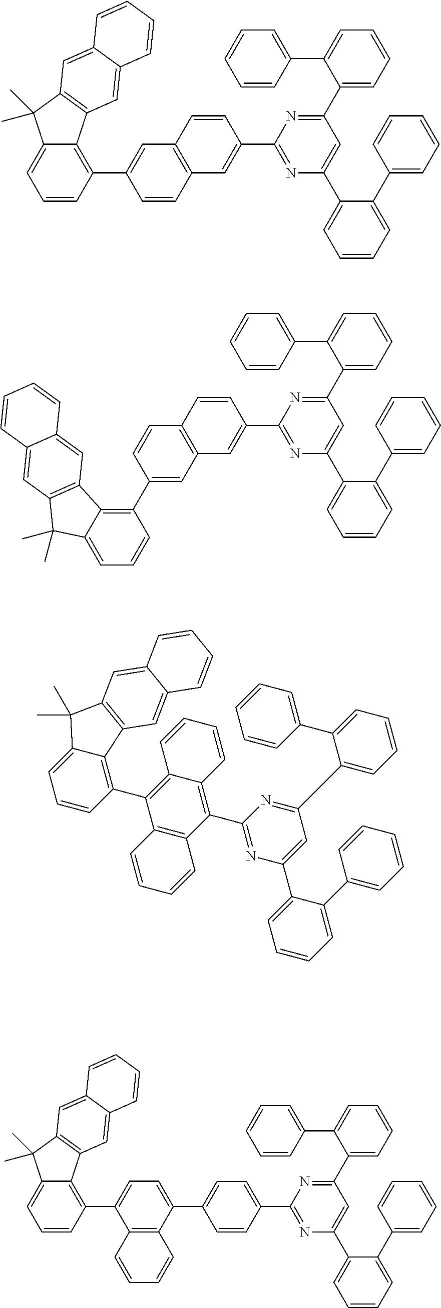 [SMILES: CC1(C)c2cc3ccccc3cc2-c2c(-c3ccc4cc(-c5nc(-c6ccccc6-c6ccccc6)cc(-c6ccccc6-c6ccccc6)n5)ccc4c3)cccc21 ; CC1(C)c2cc3ccccc3cc2-c2c(-c3ccc4ccc(-c5nc(-c6ccccc6-c6ccccc6)cc(-c6ccccc6-c6ccccc6)n5)cc4c3)cccc21 ; CC1(C)c2cc3ccccc3cc2-c2c(-c3c4ccccc4c(-c4nc(-c5ccccc5-c5ccccc5)cc(-c5ccccc5-c5ccccc5)n4)c4ccccc34)cccc21 ; CC1(C)c2cc3ccccc3cc2-c2c(-c3ccc(-c4ccc(-c5nc(-c6ccccc6-c6ccccc6)cc(-c6ccccc6-c6ccccc6)n5)cc4)c4ccccc34)cccc21]

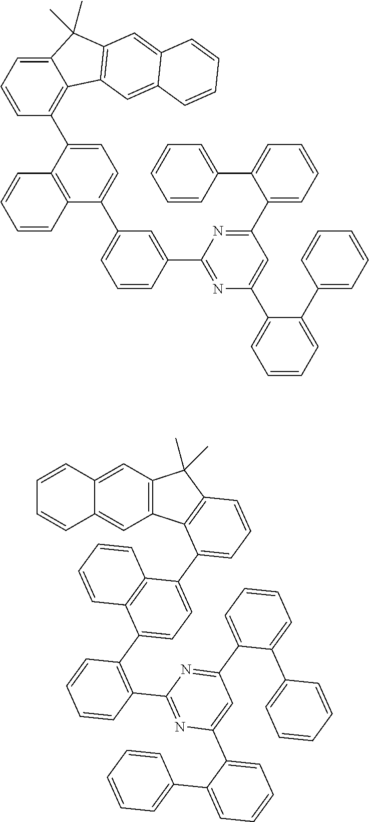 [SMILES: CC1(C)c2cc3ccccc3cc2-c2c(-c3ccc(-c4cccc(-c5nc(-c6ccccc6-c6ccccc6)cc(-c6ccccc6-c6ccccc6)n5)c4)c4ccccc34)cccc21 ; CC1(C)c2cc3ccccc3cc2-c2c(-c3ccc(-c4ccccc4-c4nc(-c5ccccc5-c5ccccc5)cc(-c5ccccc5-c5ccccc5)n4)c4ccccc34)cccc21]

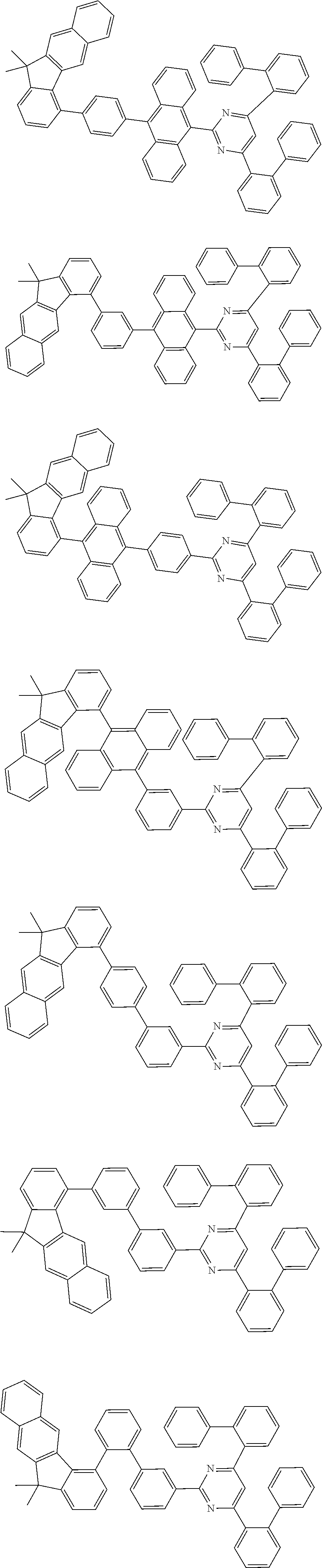

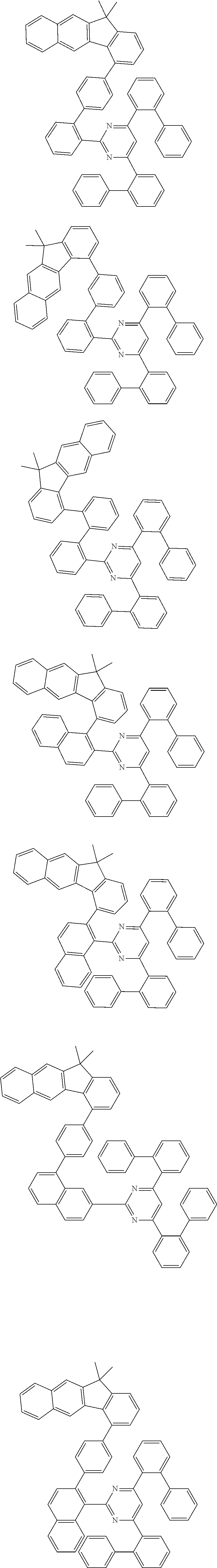

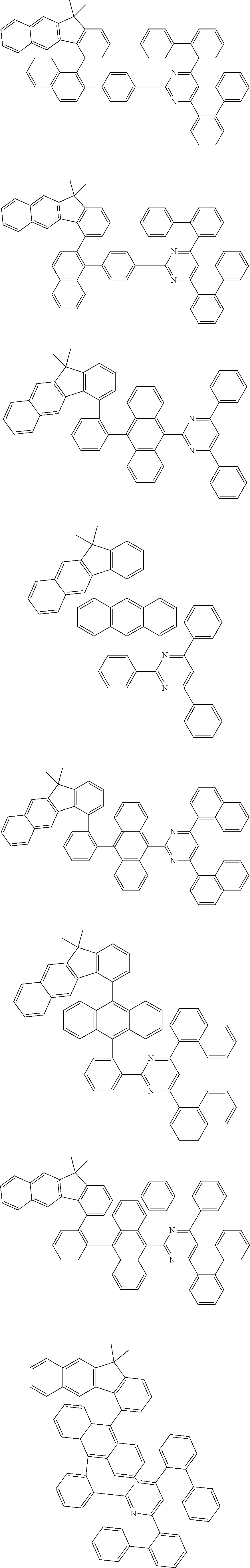

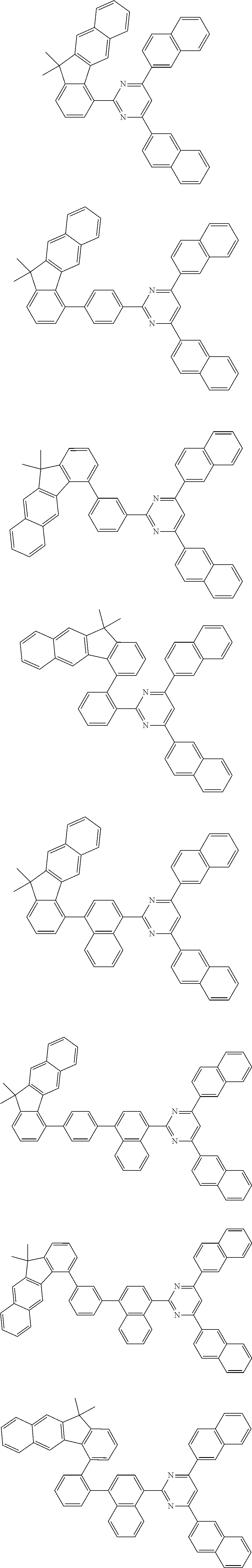

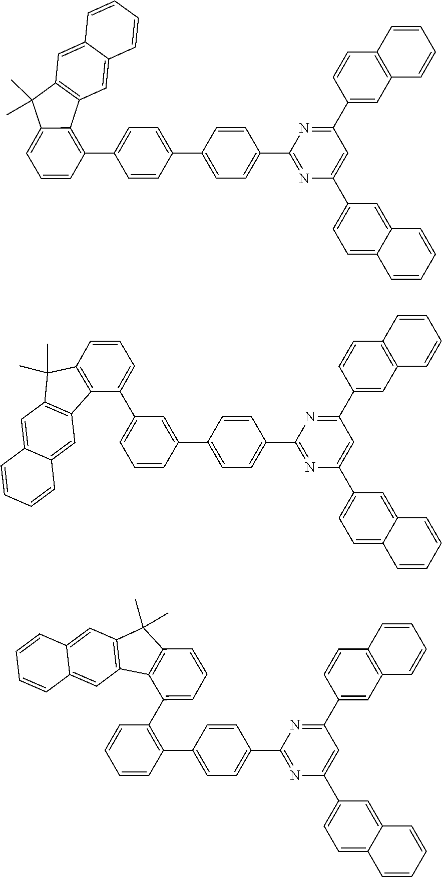

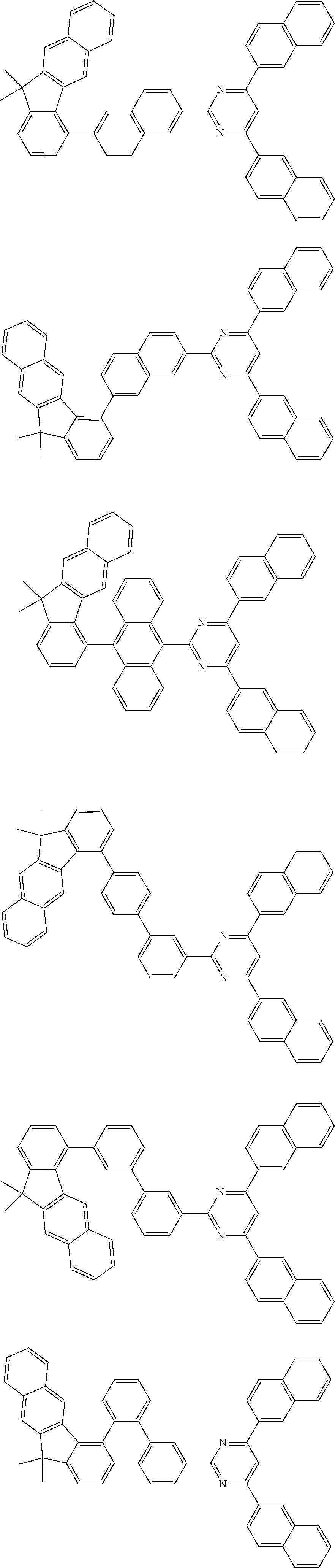

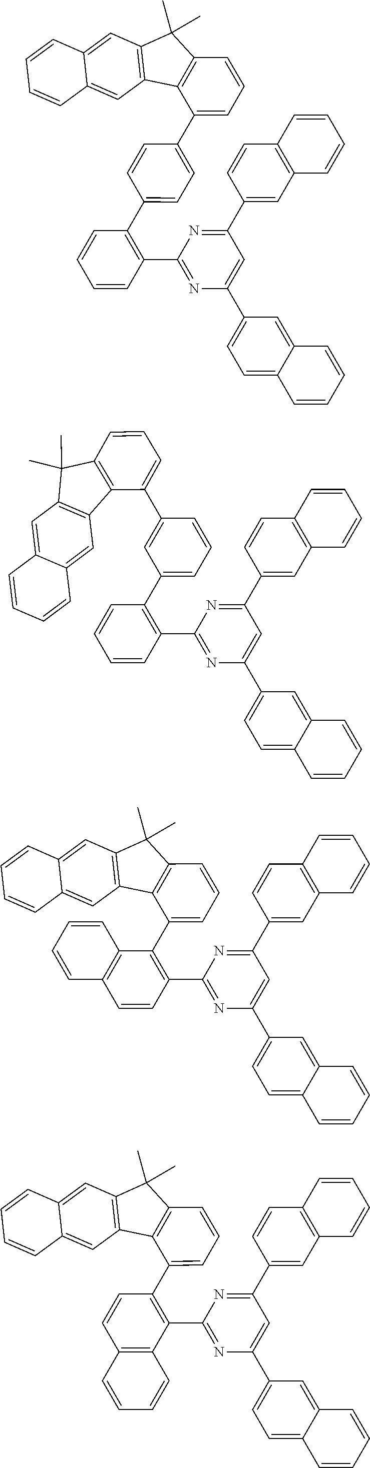 [SMILES: CC1(C)c2cc3ccccc3cc2-c2c(-c3ccc(-c4ccccc4-c4nc(-c5ccc6ccccc6c5)cc(-c5ccc6ccccc6c5)n4)cc3)cccc21 ; CC1(C)c2cc3ccccc3cc2-c2c(-c3cccc(-c4ccccc4-c4nc(-c5ccc6ccccc6c5)cc(-c5ccc6ccccc6c5)n4)c3)cccc21 ; CC1(C)c2cc3ccccc3cc2-c2c(-c3c(-c4nc(-c5ccc6ccccc6c5)cc(-c5ccc6ccccc6c5)n4)ccc4ccccc34)cccc21 ; CC1(C)c2cc3ccccc3cc2-c2c(-c3ccc4ccccc4c3-c3nc(-c4ccc5ccccc5c4)cc(-c4ccc5ccccc5c4)n3)cccc21]

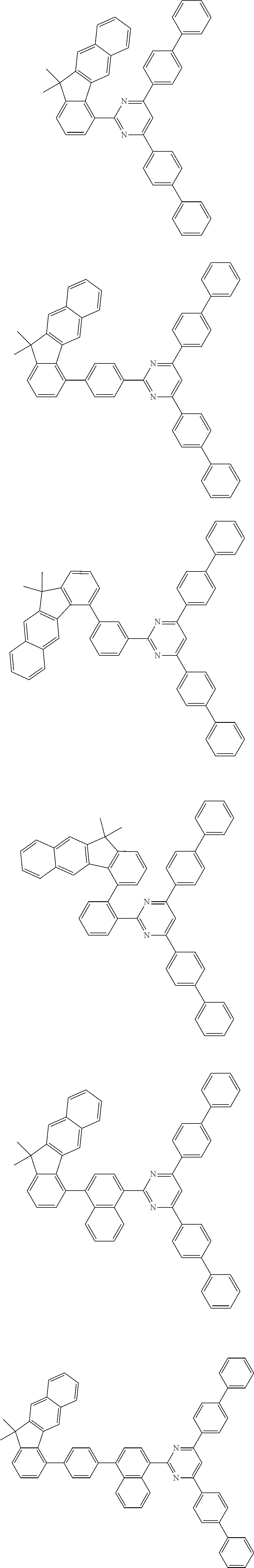

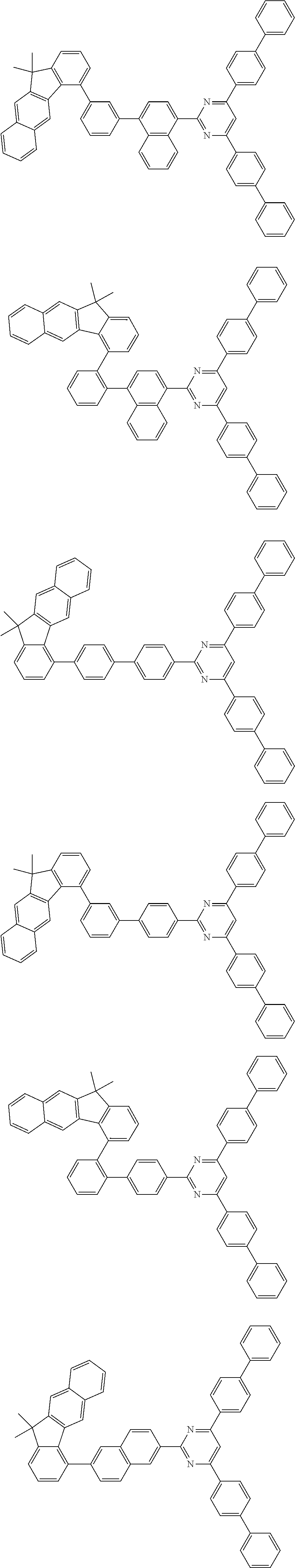

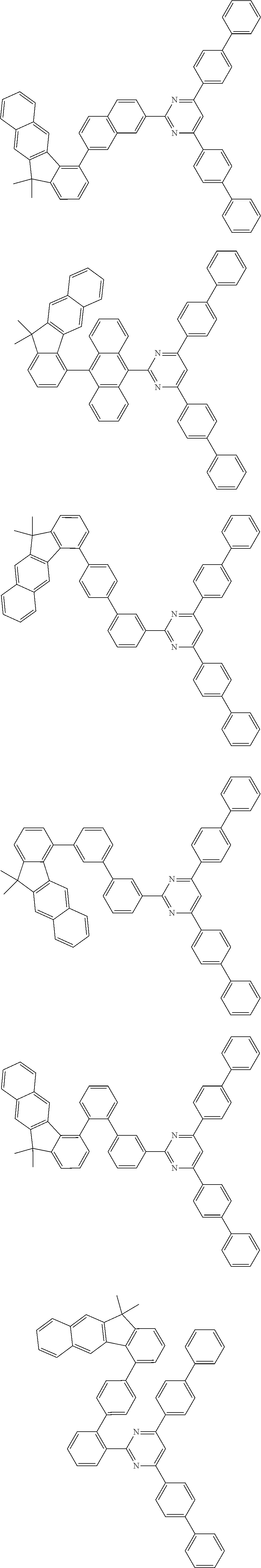

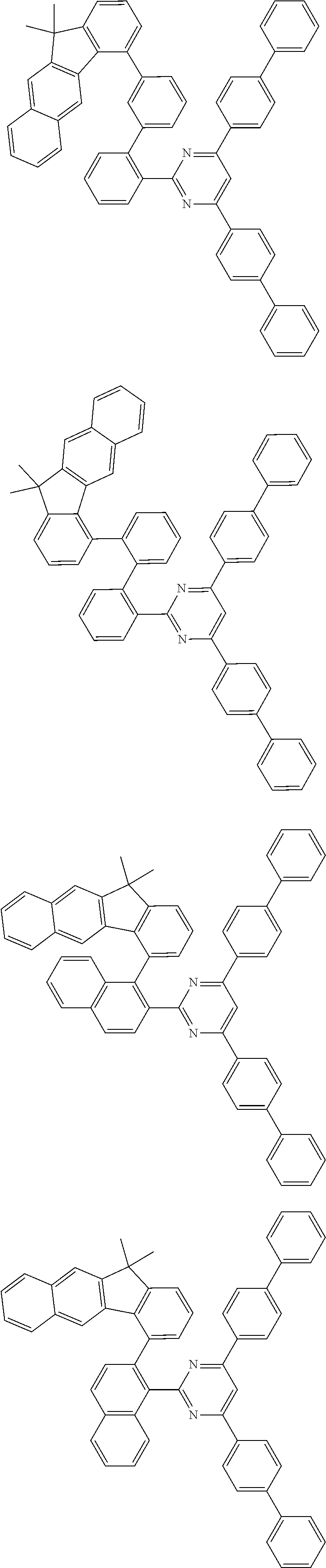

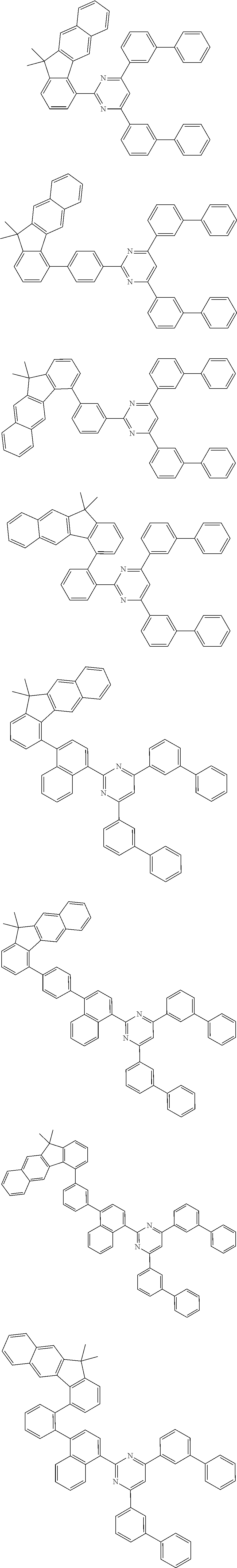

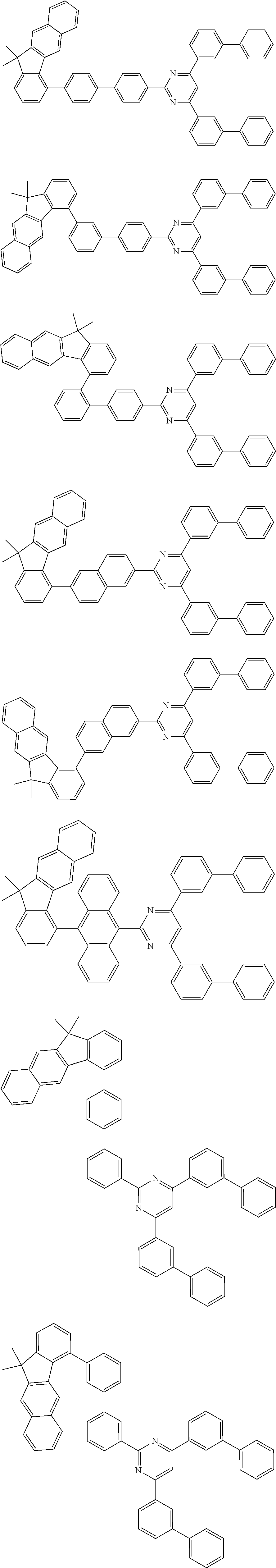

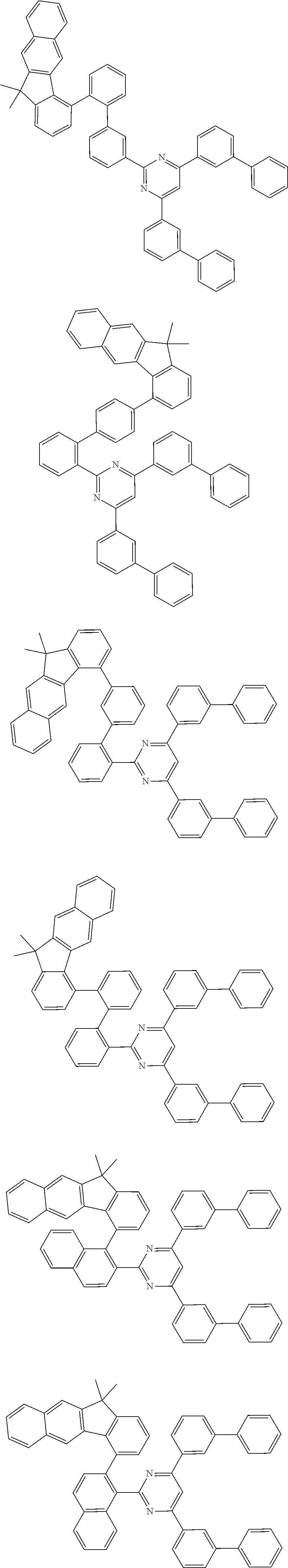

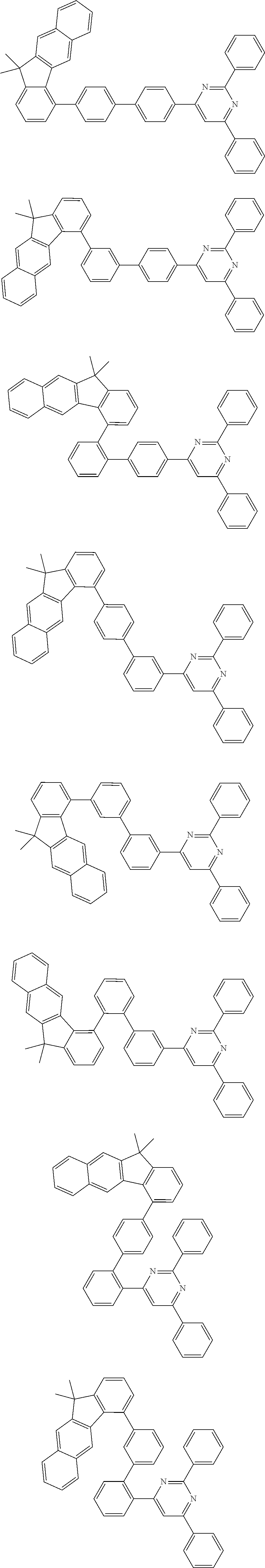

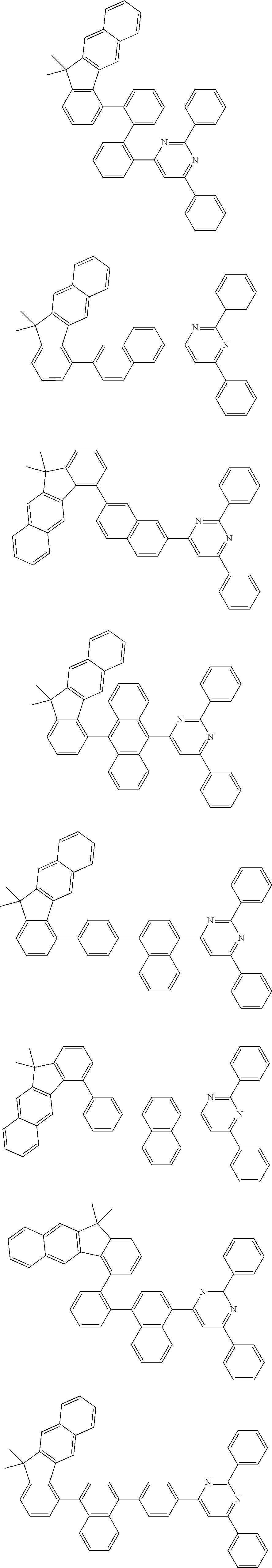

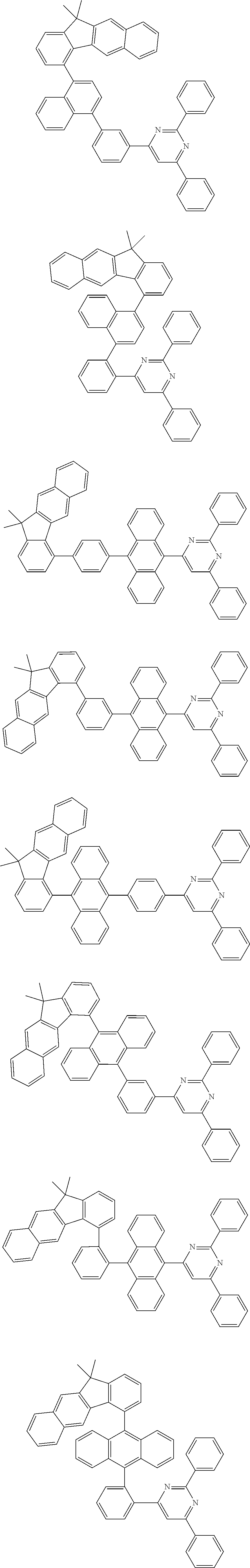

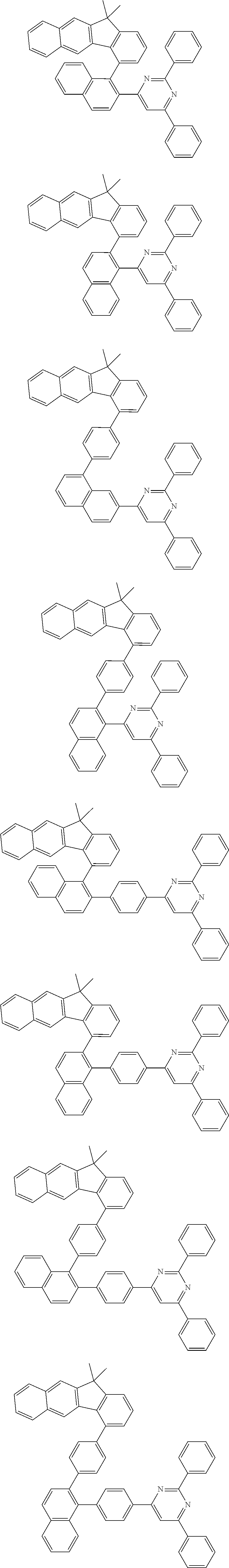

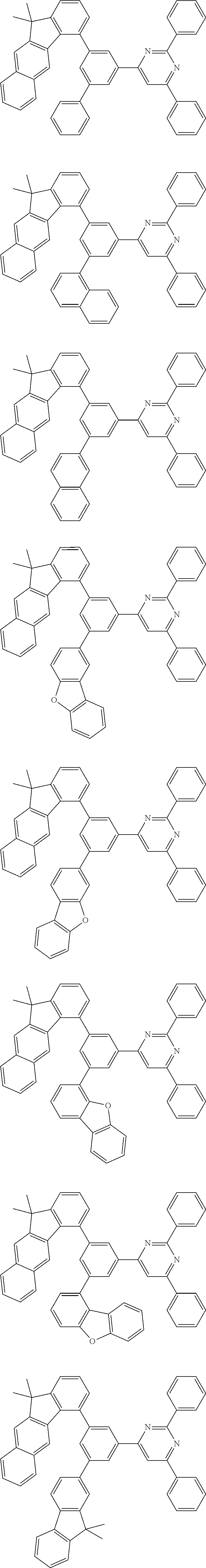

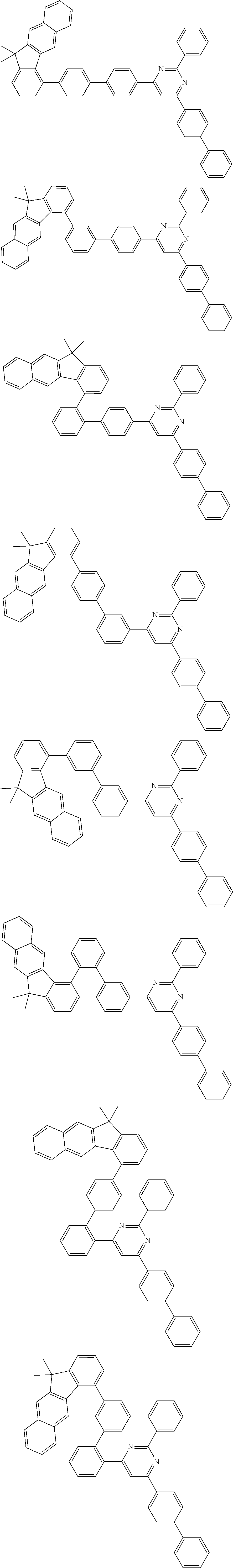

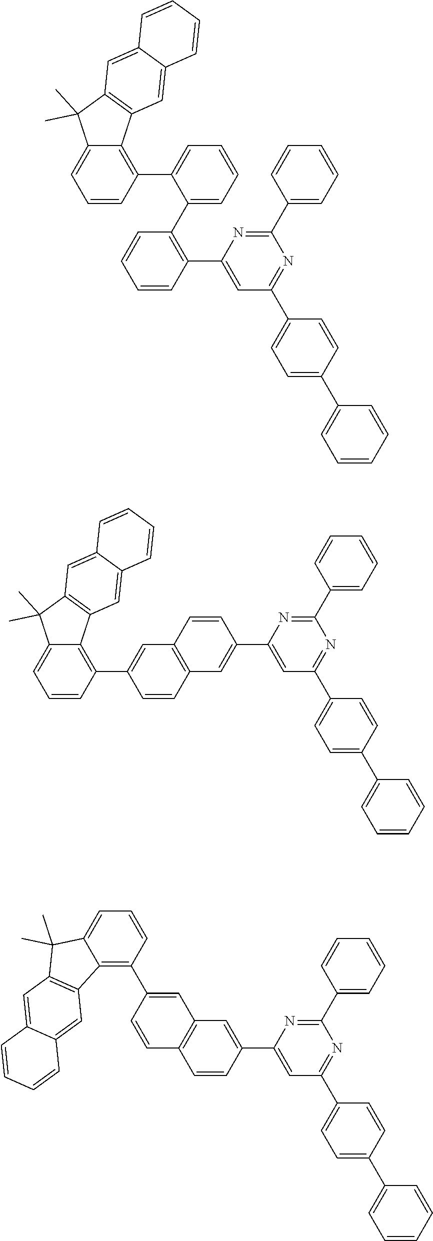 [SMILES: CC1(C)c2cc3ccccc3cc2-c2c(-c3ccccc3-c3ccccc3-c3cc(-c4ccc(-c5ccccc5)cc4)nc(-c4ccccc4)n3)cccc21 ; CC1(C)c2cc3ccccc3cc2-c2c(-c3ccc4cc(-c5cc(-c6ccc(-c7ccccc7)cc6)nc(-c6ccccc6)n5)ccc4c3)cccc21 ; CC1(C)c2cc3ccccc3cc2-c2c(-c3ccc4ccc(-c5cc(-c6ccc(-c7ccccc7)cc6)nc(-c6ccccc6)n5)cc4c3)cccc21]

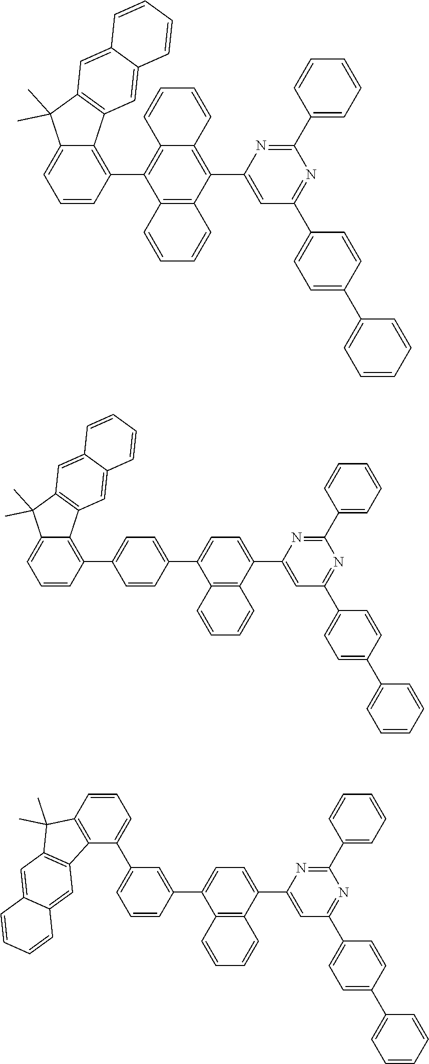 [SMILES: CC1(C)c2cc3ccccc3cc2-c2c(-c3c4ccccc4c(-c4cc(-c5ccc(-c6ccccc6)cc5)nc(-c5ccccc5)n4)c4ccccc34)cccc21 ; CC1(C)c2cc3ccccc3cc2-c2c(-c3ccc(-c4ccc(-c5cc(-c6ccc(-c7ccccc7)cc6)nc(-c6ccccc6)n5)c5ccccc45)cc3)cccc21 ; CC1(C)c2cc3ccccc3cc2-c2c(-c3cccc(-c4ccc(-c5cc(-c6ccc(-c7ccccc7)cc6)nc(-c6ccccc6)n5)c5ccccc45)c3)cccc21]

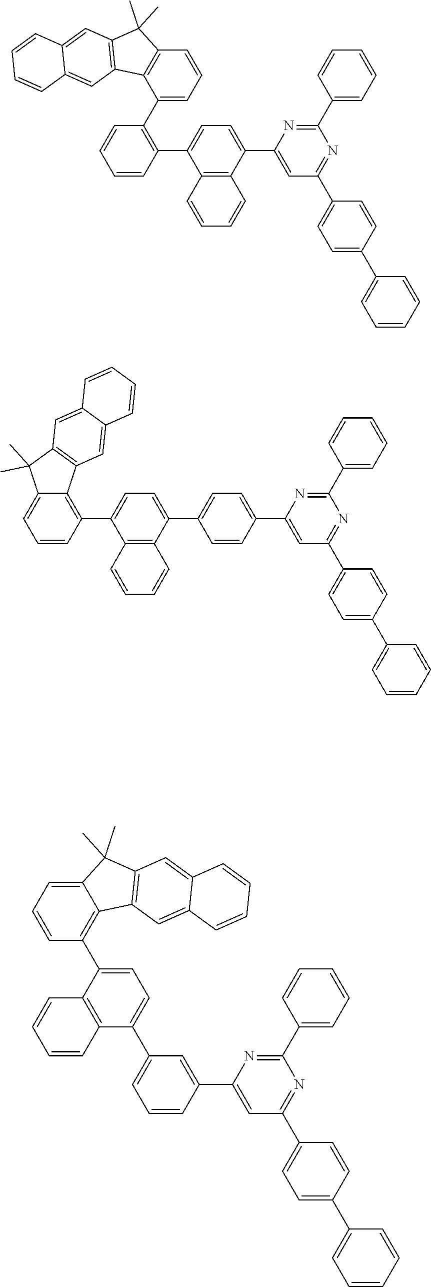 [SMILES: CC1(C)c2cc3ccccc3cc2-c2c(-c3ccccc3-c3ccc(-c4cc(-c5ccc(-c6ccccc6)cc5)nc(-c5ccccc5)n4)c4ccccc34)cccc21 ; CC1(C)c2cc3ccccc3cc2-c2c(-c3ccc(-c4ccc(-c5cc(-c6ccc(-c7ccccc7)cc6)nc(-c6ccccc6)n5)cc4)c4ccccc34)cccc21 ; CC1(C)c2cc3ccccc3cc2-c2c(-c3ccc(-c4cccc(-c5cc(-c6ccc(-c7ccccc7)cc6)nc(-c6ccccc6)n5)c4)c4ccccc34)cccc21]

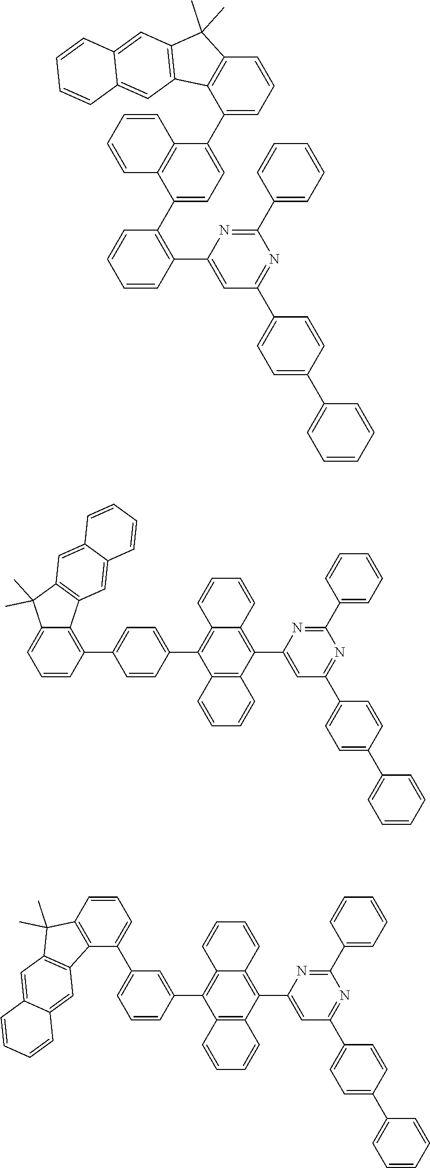 [SMILES: CC1(C)c2cc3ccccc3cc2-c2c(-c3ccc(-c4ccccc4-c4cc(-c5ccc(-c6ccccc6)cc5)nc(-c5ccccc5)n4)c4ccccc34)cccc21 ; CC1(C)c2cc3ccccc3cc2-c2c(-c3ccc(-c4c5ccccc5c(-c5cc(-c6ccc(-c7ccccc7)cc6)nc(-c6ccccc6)n5)c5ccccc45)cc3)cccc21 ; CC1(C)c2cc3ccccc3cc2-c2c(-c3cccc(-c4c5ccccc5c(-c5cc(-c6ccc(-c7ccccc7)cc6)nc(-c6ccccc6)n5)c5ccccc45)c3)cccc21]

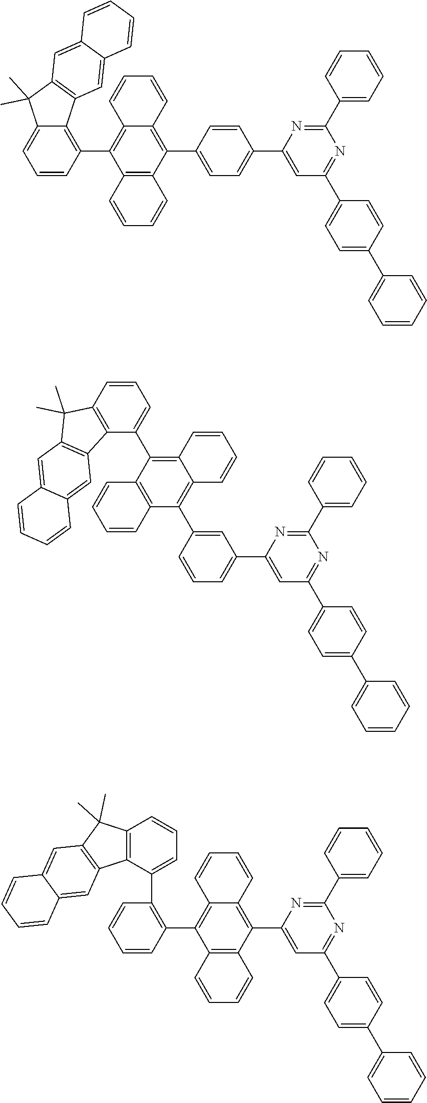 [SMILES: CC1(C)c2cc3ccccc3cc2-c2c(-c3c4ccccc4c(-c4ccc(-c5cc(-c6ccc(-c7ccccc7)cc6)nc(-c6ccccc6)n5)cc4)c4ccccc34)cccc21 ; CC1(C)c2cc3ccccc3cc2-c2c(-c3c4ccccc4c(-c4cccc(-c5cc(-c6ccc(-c7ccccc7)cc6)nc(-c6ccccc6)n5)c4)c4ccccc34)cccc21 ; CC1(C)c2cc3ccccc3cc2-c2c(-c3ccccc3-c3c4ccccc4c(-c4cc(-c5ccc(-c6ccccc6)cc5)nc(-c5ccccc5)n4)c4ccccc34)cccc21]

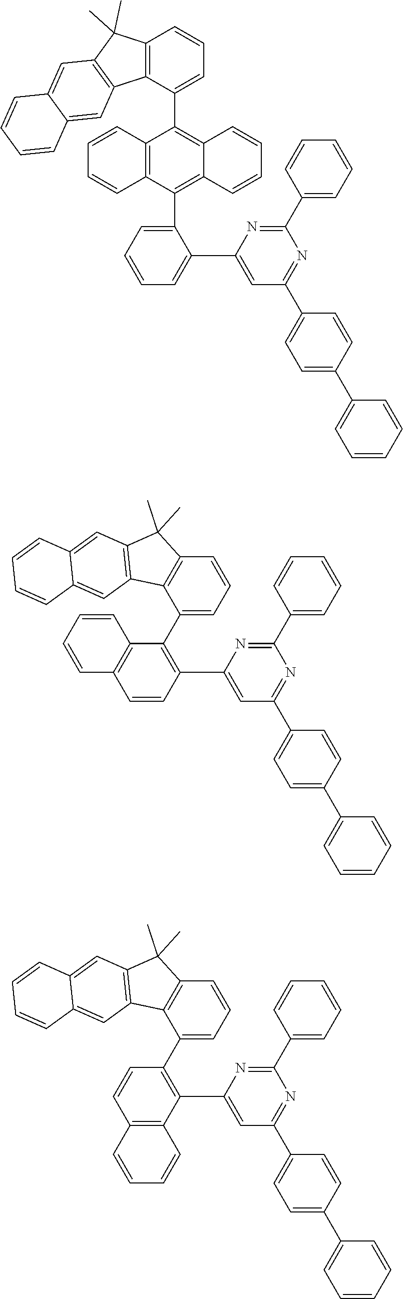 [SMILES: CC1(C)c2cc3ccccc3cc2-c2c(-c3c4ccccc4c(-c4ccccc4-c4cc(-c5ccc(-c6ccccc6)cc5)nc(-c5ccccc5)n4)c4ccccc34)cccc21 ; CC1(C)c2cc3ccccc3cc2-c2c(-c3c(-c4cc(-c5ccc(-c6ccccc6)cc5)nc(-c5ccccc5)n4)ccc4ccccc34)cccc21 ; CC1(C)c2cc3ccccc3cc2-c2c(-c3ccc4ccccc4c3-c3cc(-c4ccc(-c5ccccc5)cc4)nc(-c4ccccc4)n3)cccc21]

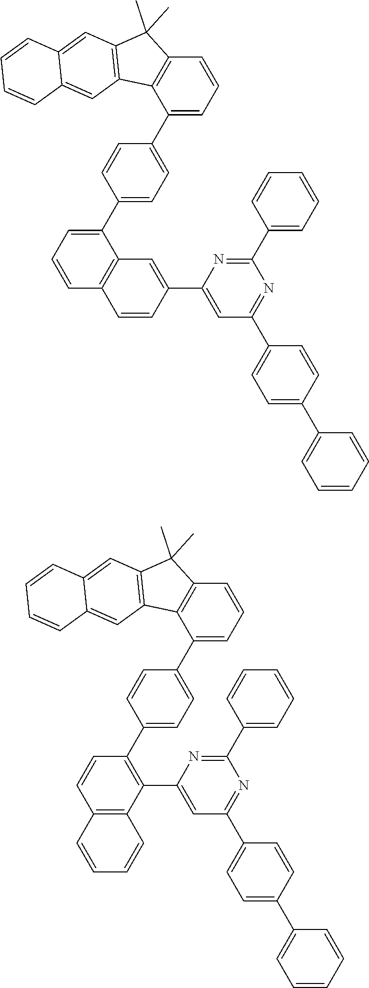 [SMILES: CC1(C)c2cc3ccccc3cc2-c2c(-c3ccc(-c4cccc5ccc(-c6cc(-c7ccc(-c8ccccc8)cc7)nc(-c7ccccc7)n6)cc45)cc3)cccc21 ; CC1(C)c2cc3ccccc3cc2-c2c(-c3ccc(-c4ccc5ccccc5c4-c4cc(-c5ccc(-c6ccccc6)cc5)nc(-c5ccccc5)n4)cc3)cccc21]

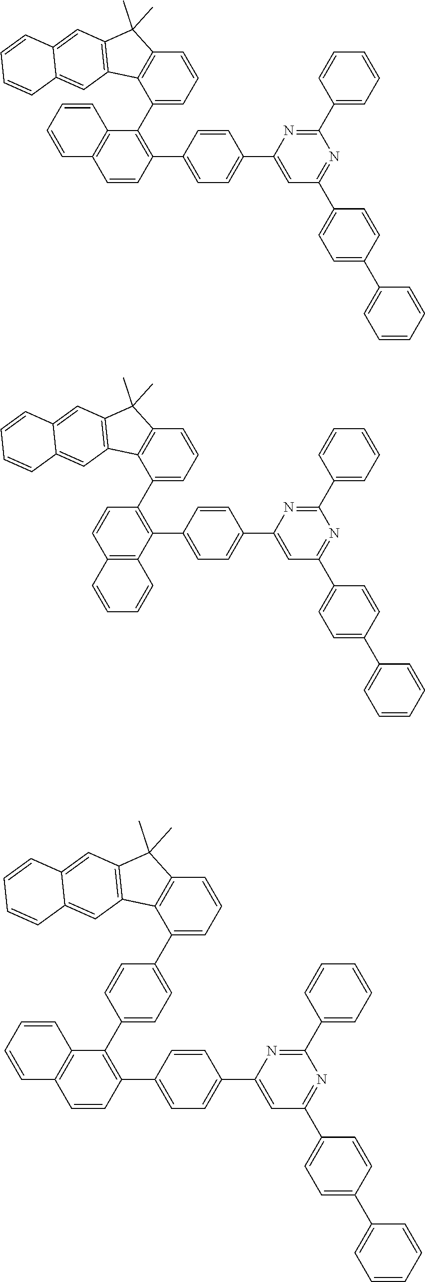 [SMILES: CC1(C)c2cc3ccccc3cc2-c2c(-c3c(-c4ccc(-c5cc(-c6ccc(-c7ccccc7)cc6)nc(-c6ccccc6)n5)cc4)ccc4ccccc34)cccc21 ; CC1(C)c2cc3ccccc3cc2-c2c(-c3ccc4ccccc4c3-c3ccc(-c4cc(-c5ccc(-c6ccccc6)cc5)nc(-c5ccccc5)n4)cc3)cccc21 ; CC1(C)c2cc3ccccc3cc2-c2c(-c3ccc(-c4c(-c5ccc(-c6cc(-c7ccc(-c8ccccc8)cc7)nc(-c7ccccc7)n6)cc5)ccc5ccccc45)cc3)cccc21]

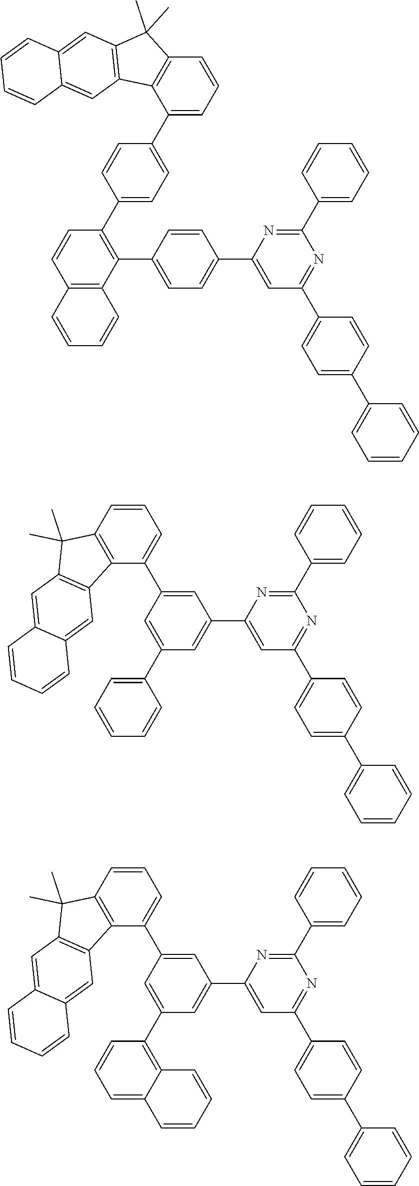 [SMILES: CC1(C)c2cc3ccccc3cc2-c2c(-c3ccc(-c4ccc5ccccc5c4-c4ccc(-c5cc(-c6ccc(-c7ccccc7)cc6)nc(-c6ccccc6)n5)cc4)cc3)cccc21 ; CC1(C)c2cc3ccccc3cc2-c2c(-c3cc(-c4ccccc4)cc(-c4cc(-c5ccc(-c6ccccc6)cc5)nc(-c5ccccc5)n4)c3)cccc21 ; CC1(C)c2cc3ccccc3cc2-c2c(-c3cc(-c4cc(-c5ccc(-c6ccccc6)cc5)nc(-c5ccccc5)n4)cc(-c4cccc5ccccc45)c3)cccc21]

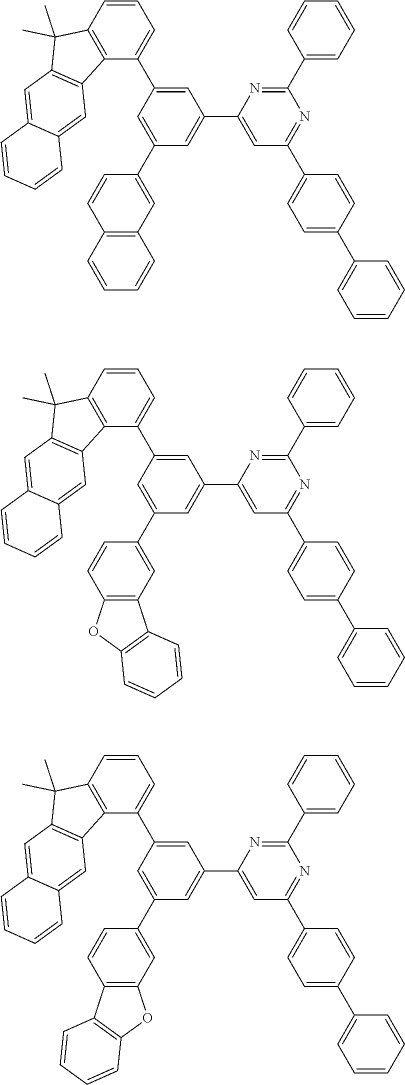 [SMILES: CC1(C)c2cc3ccccc3cc2-c2c(-c3cc(-c4ccc5ccccc5c4)cc(-c4cc(-c5ccc(-c6ccccc6)cc5)nc(-c5ccccc5)n4)c3)cccc21 ; CC1(C)c2cc3ccccc3cc2-c2c(-c3cc(-c4ccc5oc6ccccc6c5c4)cc(-c4cc(-c5ccc(-c6ccccc6)cc5)nc(-c5ccccc5)n4)c3)cccc21 ; CC1(C)c2cc3ccccc3cc2-c2c(-c3cc(-c4ccc5c(c4)oc4ccccc45)cc(-c4cc(-c5ccc(-c6ccccc6)cc5)nc(-c5ccccc5)n4)c3)cccc21]

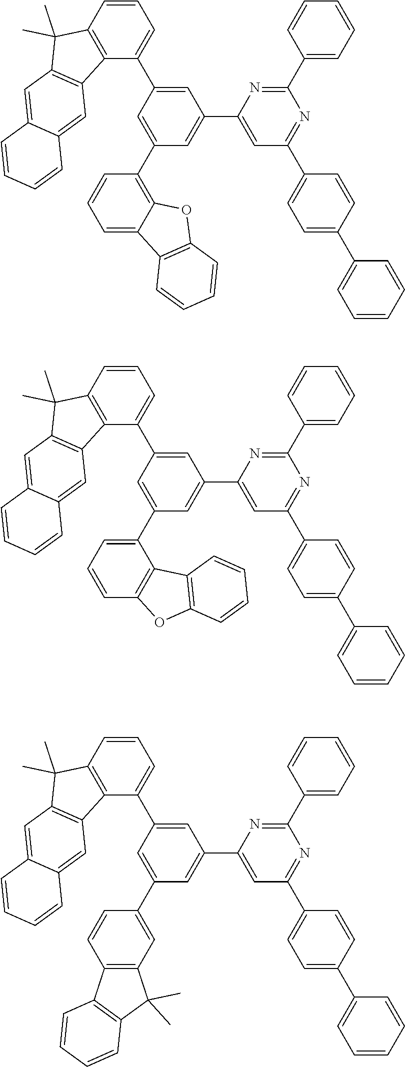 [SMILES: CC1(C)c2cc3ccccc3cc2-c2c(-c3cc(-c4cc(-c5ccc(-c6ccccc6)cc5)nc(-c5ccccc5)n4)cc(-c4cccc5c4oc4ccccc45)c3)cccc21 ; CC1(C)c2cc3ccccc3cc2-c2c(-c3cc(-c4cc(-c5ccc(-c6ccccc6)cc5)nc(-c5ccccc5)n4)cc(-c4cccc5oc6ccccc6c45)c3)cccc21 ; CC1(C)c2ccccc2-c2ccc(-c3cc(-c4cc(-c5ccc(-c6ccccc6)cc5)nc(-c5ccccc5)n4)cc(-c4cccc5c4-c4cc6ccccc6cc4C5(C)C)c3)cc21]

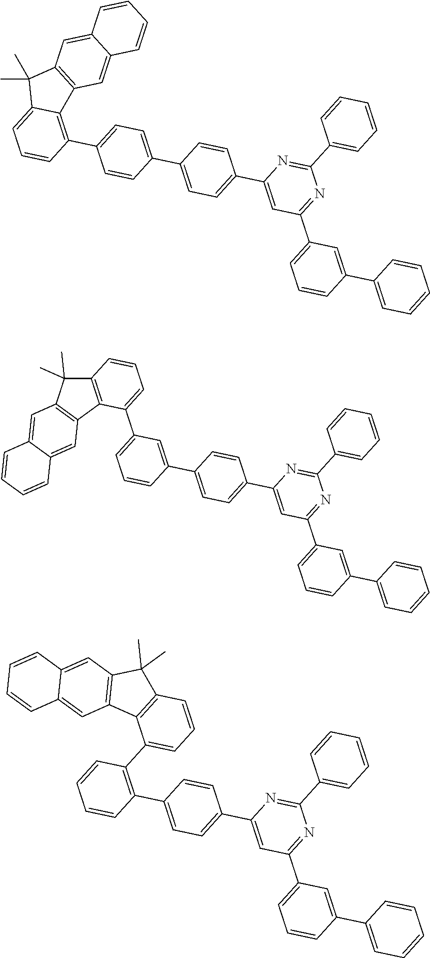 [SMILES: CC1(C)c2cc3ccccc3cc2-c2c(-c3ccc(-c4ccc(-c5cc(-c6cccc(-c7ccccc7)c6)nc(-c6ccccc6)n5)cc4)cc3)cccc21 ; CC1(C)c2cc3ccccc3cc2-c2c(-c3cccc(-c4ccc(-c5cc(-c6cccc(-c7ccccc7)c6)nc(-c6ccccc6)n5)cc4)c3)cccc21 ; CC1(C)c2cc3ccccc3cc2-c2c(-c3ccccc3-c3ccc(-c4cc(-c5cccc(-c6ccccc6)c5)nc(-c5ccccc5)n4)cc3)cccc21]

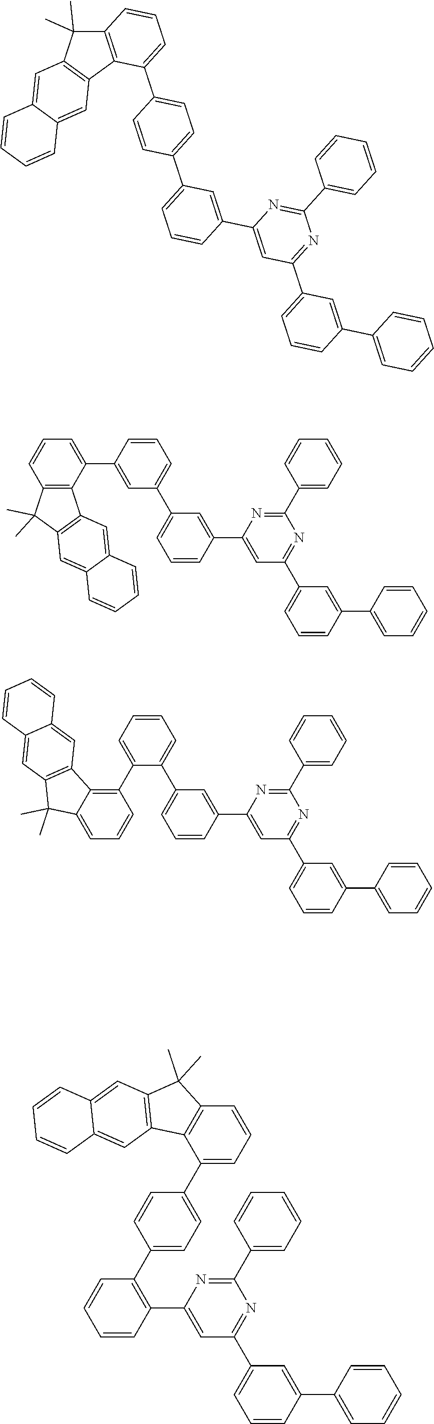 [SMILES: CC1(C)c2cc3ccccc3cc2-c2c(-c3ccc(-c4cccc(-c5cc(-c6cccc(-c7ccccc7)c6)nc(-c6ccccc6)n5)c4)cc3)cccc21 ; CC1(C)c2cc3ccccc3cc2-c2c(-c3cccc(-c4cccc(-c5cc(-c6cccc(-c7ccccc7)c6)nc(-c6ccccc6)n5)c4)c3)cccc21 ; CC1(C)c2cc3ccccc3cc2-c2c(-c3ccccc3-c3cccc(-c4cc(-c5cccc(-c6ccccc6)c5)nc(-c5ccccc5)n4)c3)cccc21 ; CC1(C)c2cc3ccccc3cc2-c2c(-c3ccc(-c4ccccc4-c4cc(-c5cccc(-c6ccccc6)c5)nc(-c5ccccc5)n4)cc3)cccc21]

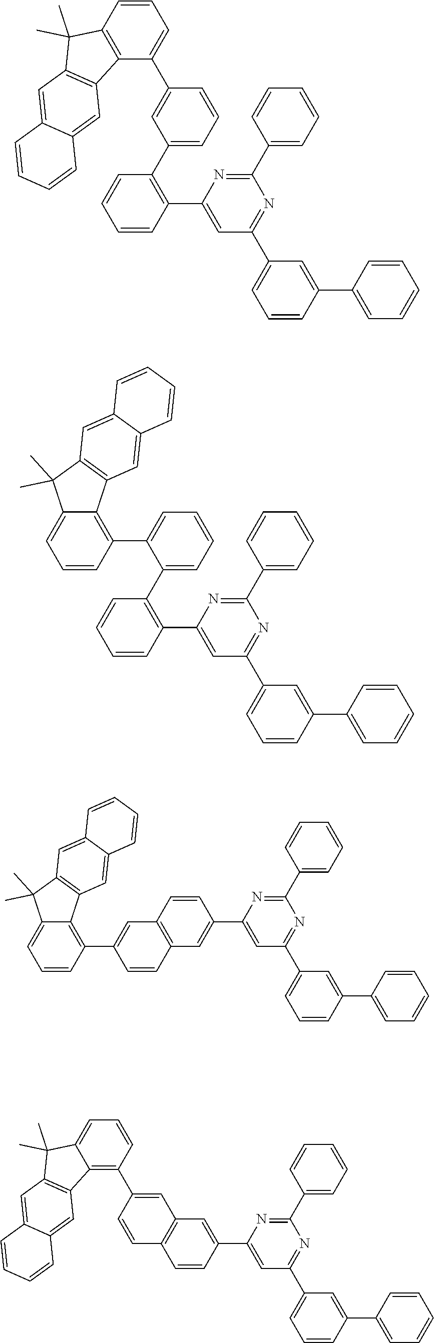 [SMILES: CC1(C)c2cc3ccccc3cc2-c2c(-c3cccc(-c4ccccc4-c4cc(-c5cccc(-c6ccccc6)c5)nc(-c5ccccc5)n4)c3)cccc21 ; CC1(C)c2cc3ccccc3cc2-c2c(-c3ccccc3-c3ccccc3-c3cc(-c4cccc(-c5ccccc5)c4)nc(-c4ccccc4)n3)cccc21 ; CC1(C)c2cc3ccccc3cc2-c2c(-c3ccc4cc(-c5cc(-c6cccc(-c7ccccc7)c6)nc(-c6ccccc6)n5)ccc4c3)cccc21 ; CC1(C)c2cc3ccccc3cc2-c2c(-c3ccc4ccc(-c5cc(-c6cccc(-c7ccccc7)c6)nc(-c6ccccc6)n5)cc4c3)cccc21]

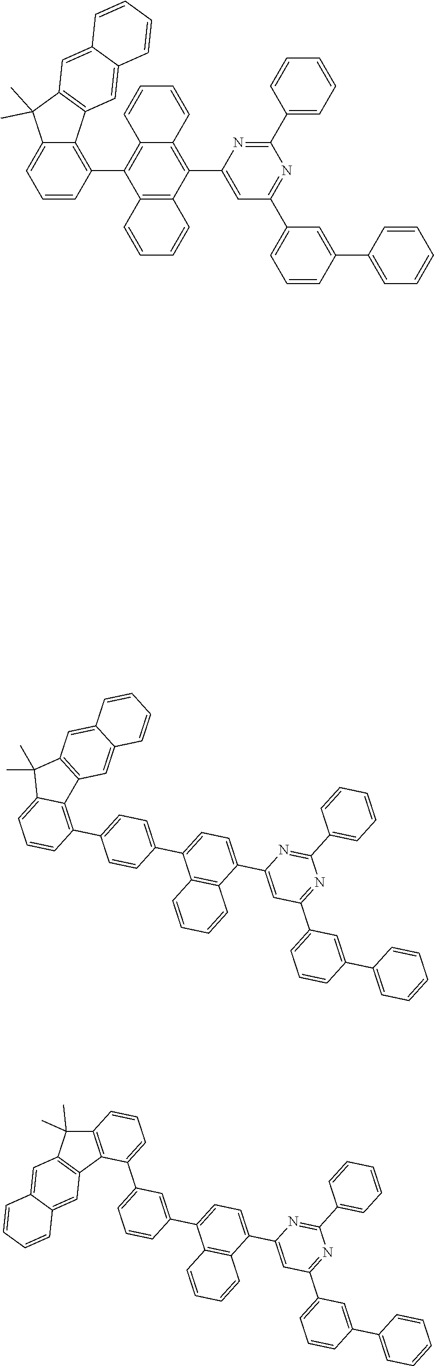 [SMILES: CC1(C)c2cc3ccccc3cc2-c2c(-c3c4ccccc4c(-c4cc(-c5cccc(-c6ccccc6)c5)nc(-c5ccccc5)n4)c4ccccc34)cccc21 ; CC1(C)c2cc3ccccc3cc2-c2c(-c3ccc(-c4ccc(-c5cc(-c6cccc(-c7ccccc7)c6)nc(-c6ccccc6)n5)c5ccccc45)cc3)cccc21 ; CC1(C)c2cc3ccccc3cc2-c2c(-c3cccc(-c4ccc(-c5cc(-c6cccc(-c7ccccc7)c6)nc(-c6ccccc6)n5)c5ccccc45)c3)cccc21]

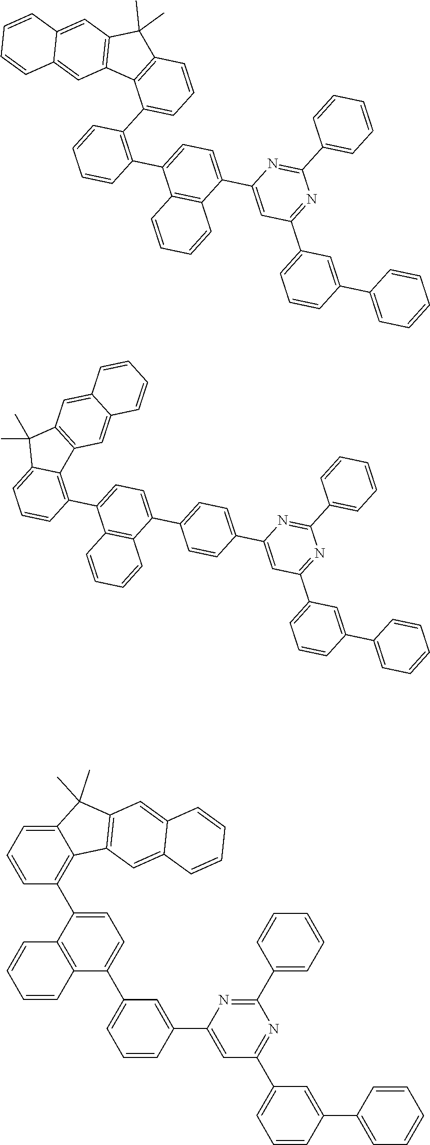 [SMILES: CC1(C)c2cc3ccccc3cc2-c2c(-c3ccccc3-c3ccc(-c4cc(-c5cccc(-c6ccccc6)c5)nc(-c5ccccc5)n4)c4ccccc34)cccc21 ; CC1(C)c2cc3ccccc3cc2-c2c(-c3ccc(-c4ccc(-c5cc(-c6cccc(-c7ccccc7)c6)nc(-c6ccccc6)n5)cc4)c4ccccc34)cccc21 ; CC1(C)c2cc3ccccc3cc2-c2c(-c3ccc(-c4cccc(-c5cc(-c6cccc(-c7ccccc7)c6)nc(-c6ccccc6)n5)c4)c4ccccc34)cccc21]

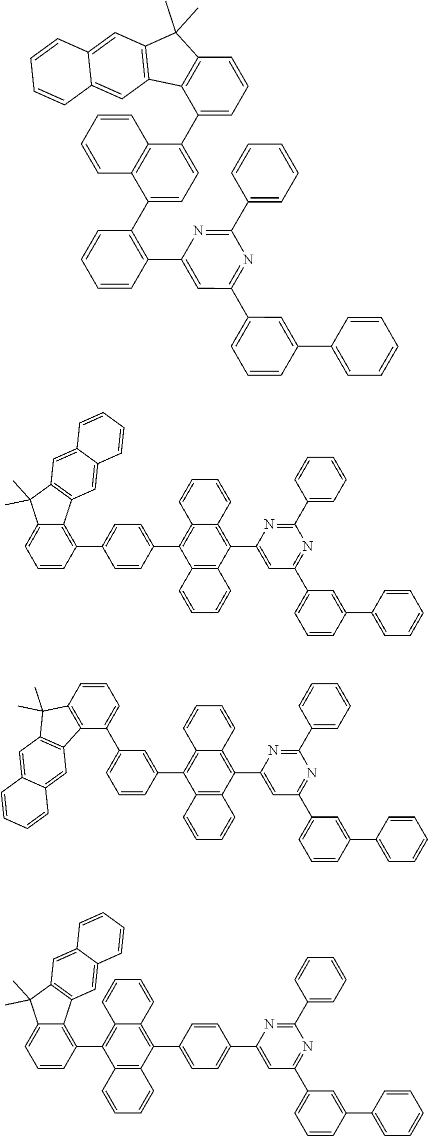 [SMILES: CC1(C)c2cc3ccccc3cc2-c2c(-c3ccc(-c4ccccc4-c4cc(-c5cccc(-c6ccccc6)c5)nc(-c5ccccc5)n4)c4ccccc34)cccc21 ; CC1(C)c2cc3ccccc3cc2-c2c(-c3ccc(-c4c5ccccc5c(-c5cc(-c6cccc(-c7ccccc7)c6)nc(-c6ccccc6)n5)c5ccccc45)cc3)cccc21 ; CC1(C)c2cc3ccccc3cc2-c2c(-c3cccc(-c4c5ccccc5c(-c5cc(-c6cccc(-c7ccccc7)c6)nc(-c6ccccc6)n5)c5ccccc45)c3)cccc21 ; CC1(C)c2cc3ccccc3cc2-c2c(-c3c4ccccc4c(-c4ccc(-c5cc(-c6cccc(-c7ccccc7)c6)nc(-c6ccccc6)n5)cc4)c4ccccc34)cccc21]

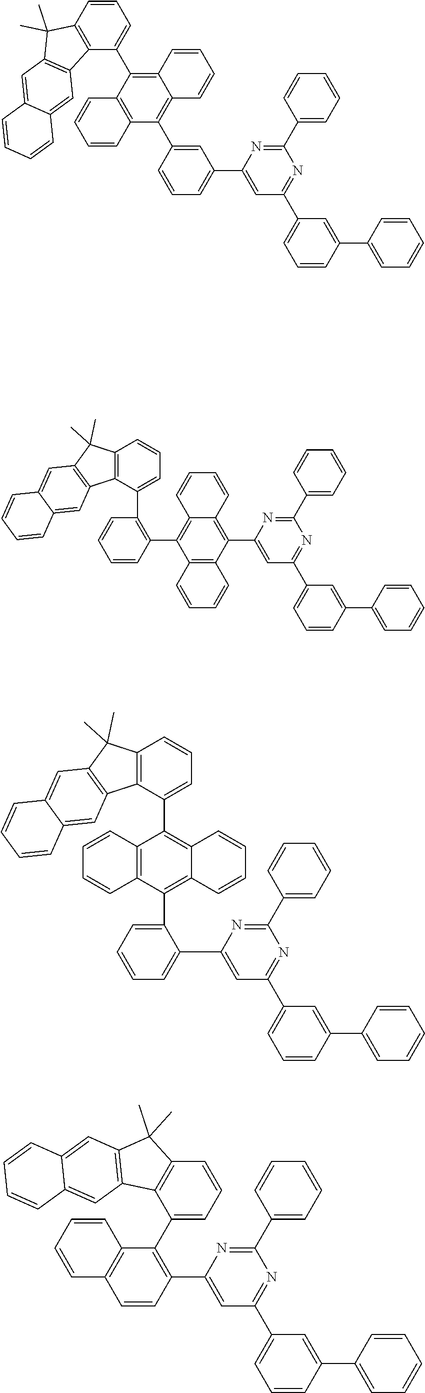 [SMILES: CC1(C)c2cc3ccccc3cc2-c2c(-c3c4ccccc4c(-c4cccc(-c5cc(-c6cccc(-c7ccccc7)c6)nc(-c6ccccc6)n5)c4)c4ccccc34)cccc21 ; CC1(C)c2cc3ccccc3cc2-c2c(-c3ccccc3-c3c4ccccc4c(-c4cc(-c5cccc(-c6ccccc6)c5)nc(-c5ccccc5)n4)c4ccccc34)cccc21 ; CC1(C)c2cc3ccccc3cc2-c2c(-c3c4ccccc4c(-c4ccccc4-c4cc(-c5cccc(-c6ccccc6)c5)nc(-c5ccccc5)n4)c4ccccc34)cccc21 ; CC1(C)c2cc3ccccc3cc2-c2c(-c3c(-c4cc(-c5cccc(-c6ccccc6)c5)nc(-c5ccccc5)n4)ccc4ccccc34)cccc21]

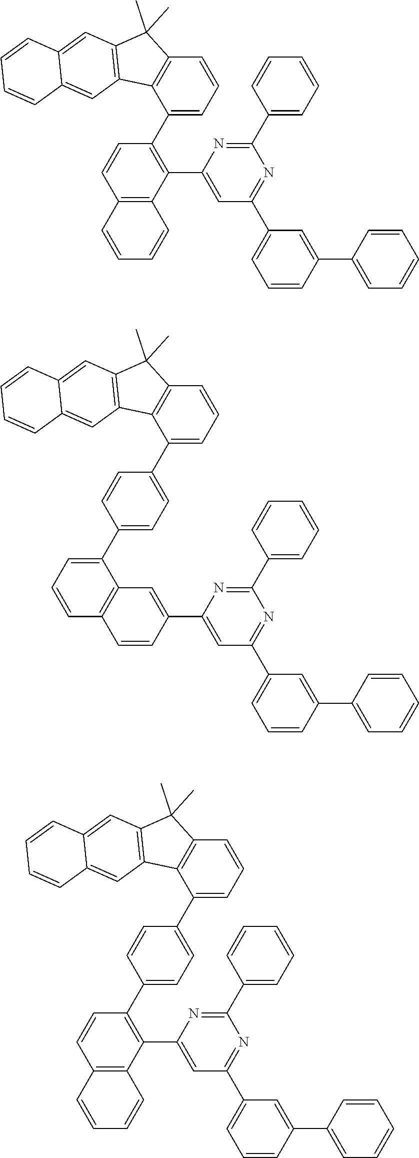 [SMILES: CC1(C)c2cc3ccccc3cc2-c2c(-c3ccc4ccccc4c3-c3cc(-c4cccc(-c5ccccc5)c4)nc(-c4ccccc4)n3)cccc21 ; CC1(C)c2cc3ccccc3cc2-c2c(-c3ccc(-c4cccc5ccc(-c6cc(-c7cccc(-c8ccccc8)c7)nc(-c7ccccc7)n6)cc45)cc3)cccc21 ; CC1(C)c2cc3ccccc3cc2-c2c(-c3ccc(-c4ccc5ccccc5c4-c4cc(-c5cccc(-c6ccccc6)c5)nc(-c5ccccc5)n4)cc3)cccc21]

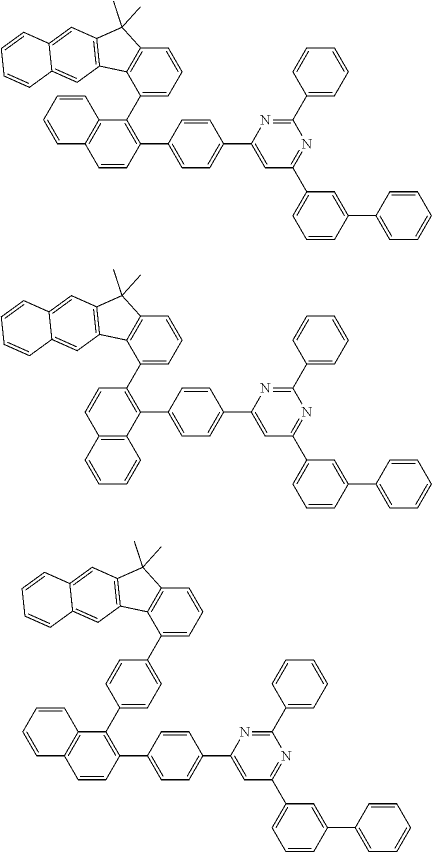 [SMILES: CC1(C)c2cc3ccccc3cc2-c2c(-c3c(-c4ccc(-c5cc(-c6cccc(-c7ccccc7)c6)nc(-c6ccccc6)n5)cc4)ccc4ccccc34)cccc21 ; CC1(C)c2cc3ccccc3cc2-c2c(-c3ccc4ccccc4c3-c3ccc(-c4cc(-c5cccc(-c6ccccc6)c5)nc(-c5ccccc5)n4)cc3)cccc21 ; CC1(C)c2cc3ccccc3cc2-c2c(-c3ccc(-c4c(-c5ccc(-c6cc(-c7cccc(-c8ccccc8)c7)nc(-c7ccccc7)n6)cc5)ccc5ccccc45)cc3)cccc21]

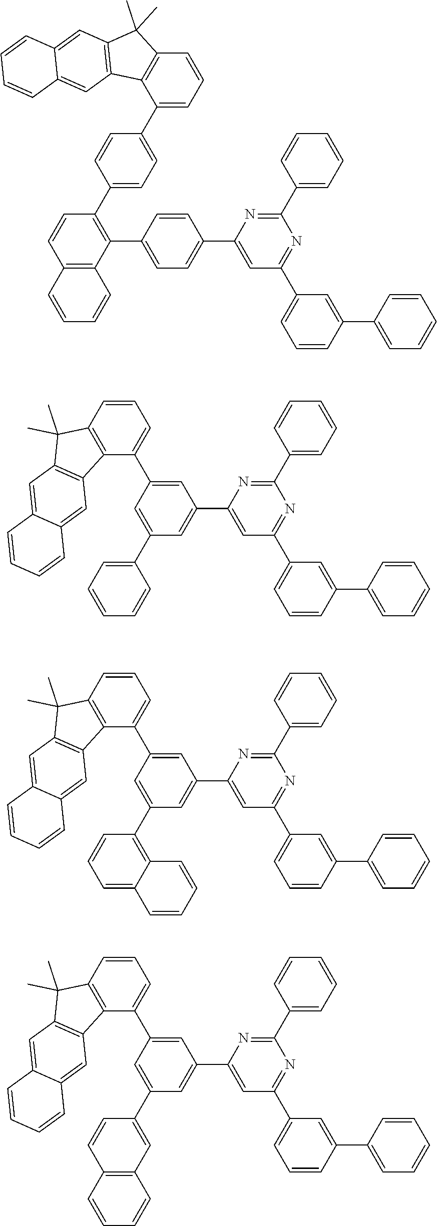 [SMILES: CC1(C)c2cc3ccccc3cc2-c2c(-c3ccc(-c4ccc5ccccc5c4-c4ccc(-c5cc(-c6cccc(-c7ccccc7)c6)nc(-c6ccccc6)n5)cc4)cc3)cccc21 ; CC1(C)c2cc3ccccc3cc2-c2c(-c3cc(-c4ccccc4)cc(-c4cc(-c5cccc(-c6ccccc6)c5)nc(-c5ccccc5)n4)c3)cccc21 ; CC1(C)c2cc3ccccc3cc2-c2c(-c3cc(-c4cc(-c5cccc(-c6ccccc6)c5)nc(-c5ccccc5)n4)cc(-c4cccc5ccccc45)c3)cccc21 ; CC1(C)c2cc3ccccc3cc2-c2c(-c3cc(-c4ccc5ccccc5c4)cc(-c4cc(-c5cccc(-c6ccccc6)c5)nc(-c5ccccc5)n4)c3)cccc21]

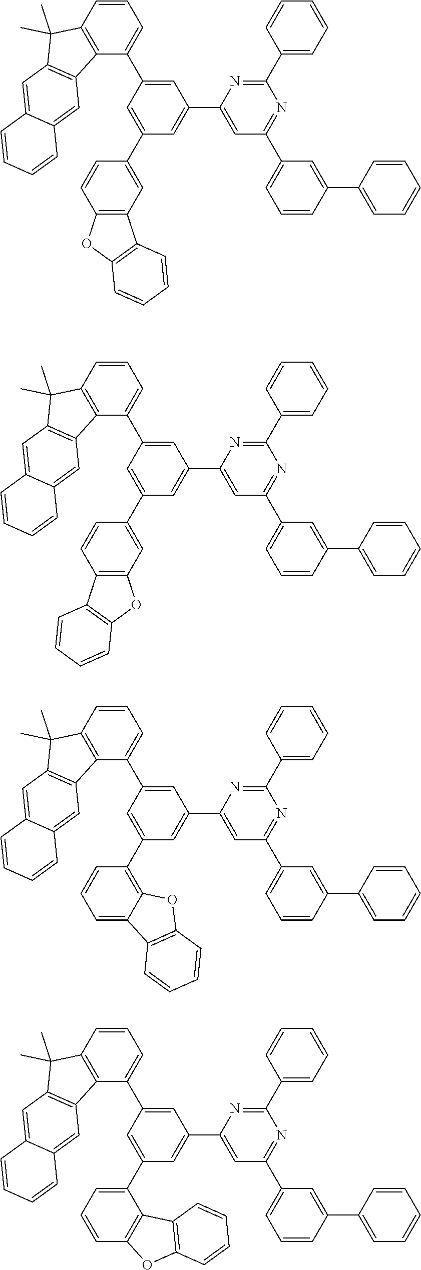 [SMILES: CC1(C)c2cc3ccccc3cc2-c2c(-c3cc(-c4ccc5oc6ccccc6c5c4)cc(-c4cc(-c5cccc(-c6ccccc6)c5)nc(-c5ccccc5)n4)c3)cccc21 ; CC1(C)c2cc3ccccc3cc2-c2c(-c3cc(-c4ccc5c(c4)oc4ccccc45)cc(-c4cc(-c5cccc(-c6ccccc6)c5)nc(-c5ccccc5)n4)c3)cccc21 ; CC1(C)c2cc3ccccc3cc2-c2c(-c3cc(-c4cc(-c5cccc(-c6ccccc6)c5)nc(-c5ccccc5)n4)cc(-c4cccc5c4oc4ccccc45)c3)cccc21 ; CC1(C)c2cc3ccccc3cc2-c2c(-c3cc(-c4cc(-c5cccc(-c6ccccc6)c5)nc(-c5ccccc5)n4)cc(-c4cccc5oc6ccccc6c45)c3)cccc21]

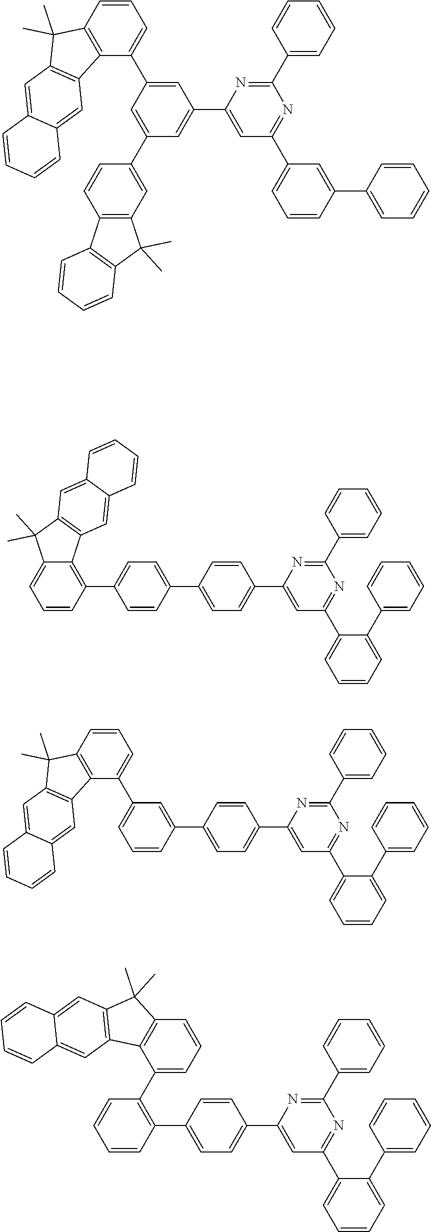 [SMILES: CC1(C)c2ccccc2-c2ccc(-c3cc(-c4cc(-c5cccc(-c6ccccc6)c5)nc(-c5ccccc5)n4)cc(-c4cccc5c4-c4cc6ccccc6cc4C5(C)C)c3)cc21 ; CC1(C)c2cc3ccccc3cc2-c2c(-c3ccc(-c4ccc(-c5cc(-c6ccccc6-c6ccccc6)nc(-c6ccccc6)n5)cc4)cc3)cccc21 ; CC1(C)c2cc3ccccc3cc2-c2c(-c3cccc(-c4ccc(-c5cc(-c6ccccc6-c6ccccc6)nc(-c6ccccc6)n5)cc4)c3)cccc21 ; CC1(C)c2cc3ccccc3cc2-c2c(-c3ccccc3-c3ccc(-c4cc(-c5ccccc5-c5ccccc5)nc(-c5ccccc5)n4)cc3)cccc21]

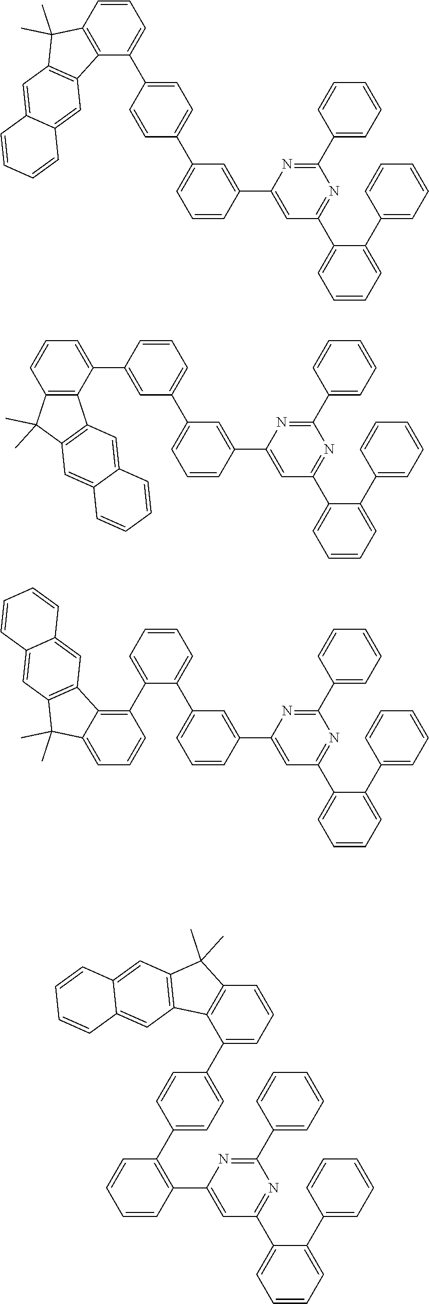

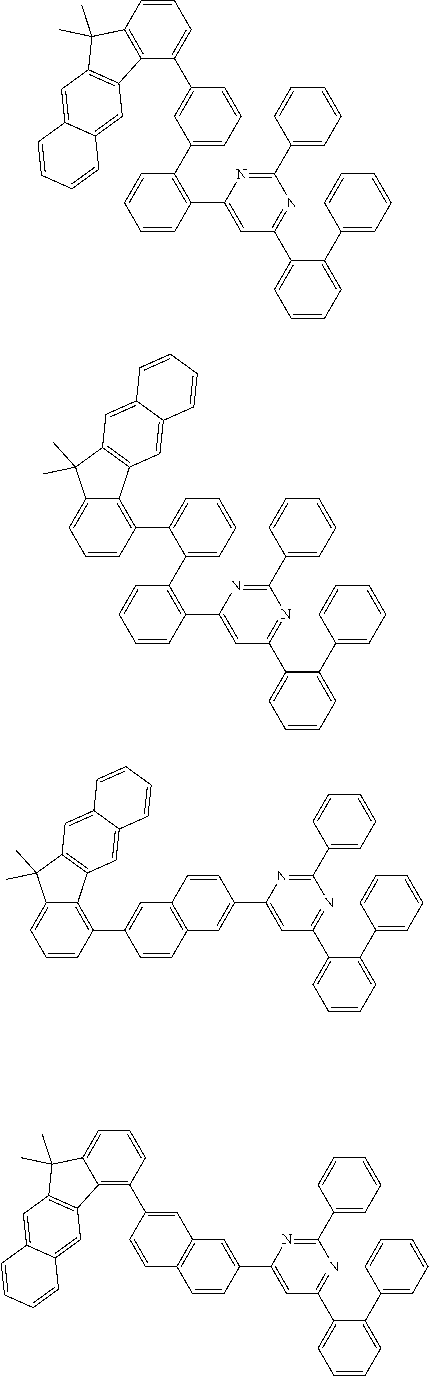 [SMILES: CC1(C)c2cc3ccccc3cc2-c2c(-c3cccc(-c4ccccc4-c4cc(-c5ccccc5-c5ccccc5)nc(-c5ccccc5)n4)c3)cccc21 ; CC1(C)c2cc3ccccc3cc2-c2c(-c3ccccc3-c3ccccc3-c3cc(-c4ccccc4-c4ccccc4)nc(-c4ccccc4)n3)cccc21 ; CC1(C)c2cc3ccccc3cc2-c2c(-c3ccc4cc(-c5cc(-c6ccccc6-c6ccccc6)nc(-c6ccccc6)n5)ccc4c3)cccc21 ; CC1(C)c2cc3ccccc3cc2-c2c(-c3ccc4ccc(-c5cc(-c6ccccc6-c6ccccc6)nc(-c6ccccc6)n5)cc4c3)cccc21]

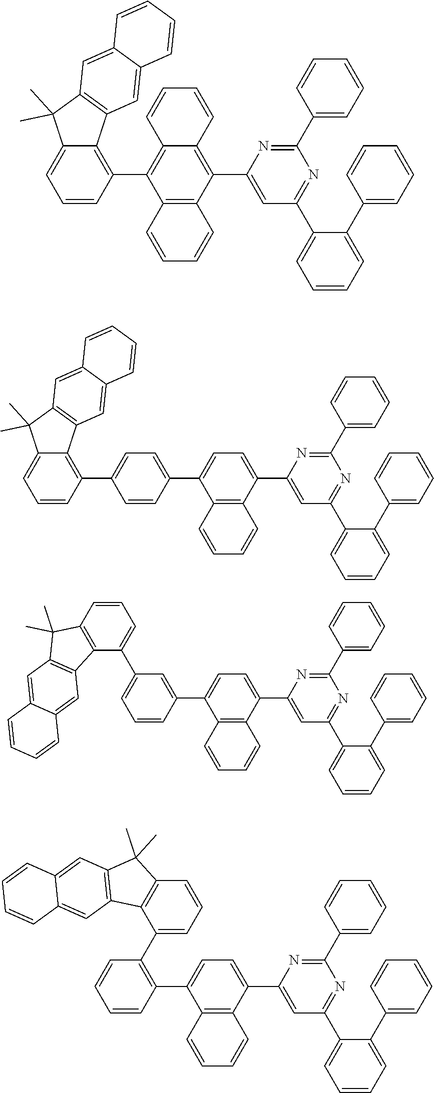

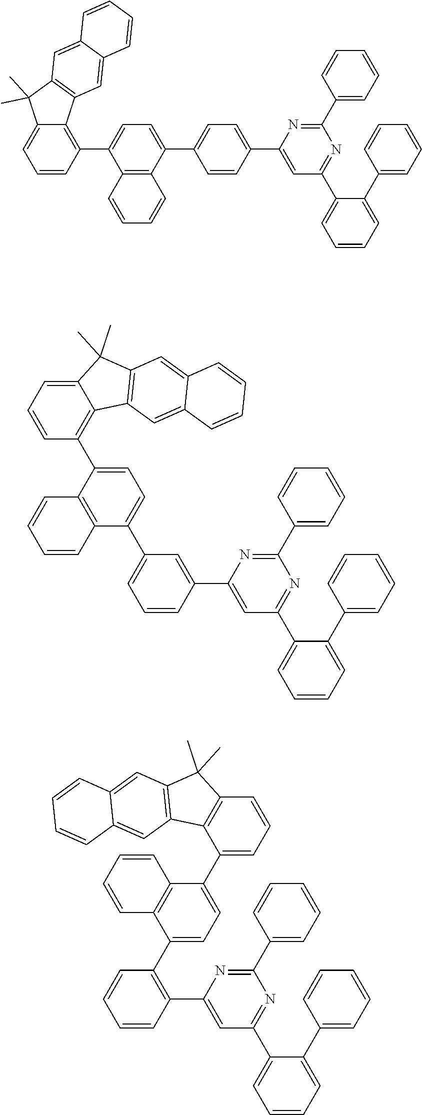 [SMILES: CC1(C)c2cc3ccccc3cc2-c2c(-c3ccc(-c4ccc(-c5cc(-c6ccccc6-c6ccccc6)nc(-c6ccccc6)n5)cc4)c4ccccc34)cccc21 ; CC1(C)c2cc3ccccc3cc2-c2c(-c3ccc(-c4cccc(-c5cc(-c6ccccc6-c6ccccc6)nc(-c6ccccc6)n5)c4)c4ccccc34)cccc21 ; CC1(C)c2cc3ccccc3cc2-c2c(-c3ccc(-c4ccccc4-c4cc(-c5ccccc5-c5ccccc5)nc(-c5ccccc5)n4)c4ccccc34)cccc21]

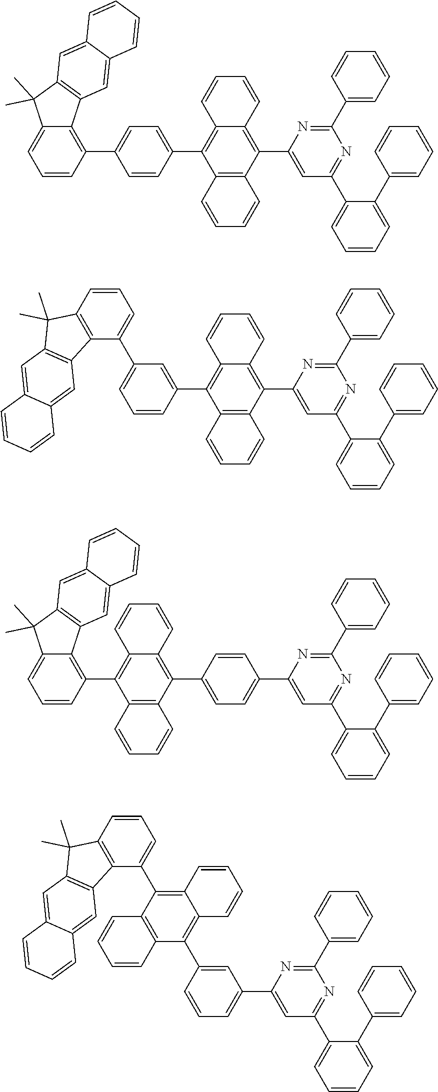

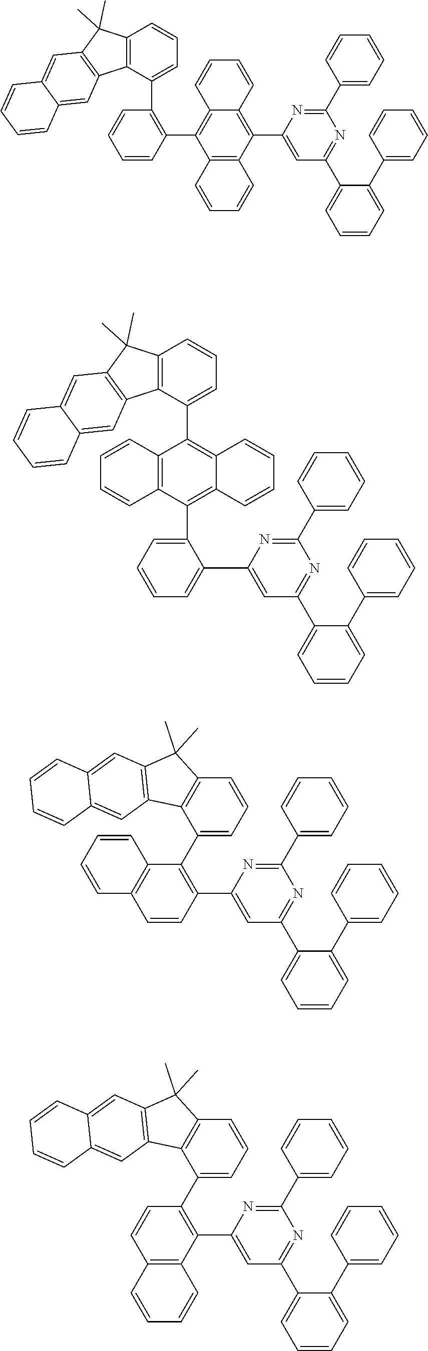 [SMILES: CC1(C)c2cc3ccccc3cc2-c2c(-c3ccccc3-c3c4ccccc4c(-c4cc(-c5ccccc5-c5ccccc5)nc(-c5ccccc5)n4)c4ccccc34)cccc21 ; CC1(C)c2cc3ccccc3cc2-c2c(-c3c4ccccc4c(-c4ccccc4-c4cc(-c5ccccc5-c5ccccc5)nc(-c5ccccc5)n4)c4ccccc34)cccc21 ; CC1(C)c2cc3ccccc3cc2-c2c(-c3c(-c4cc(-c5ccccc5-c5ccccc5)nc(-c5ccccc5)n4)ccc4ccccc34)cccc21 ; CC1(C)c2cc3ccccc3cc2-c2c(-c3ccc4ccccc4c3-c3cc(-c4ccccc4-c4ccccc4)nc(-c4ccccc4)n3)cccc21]

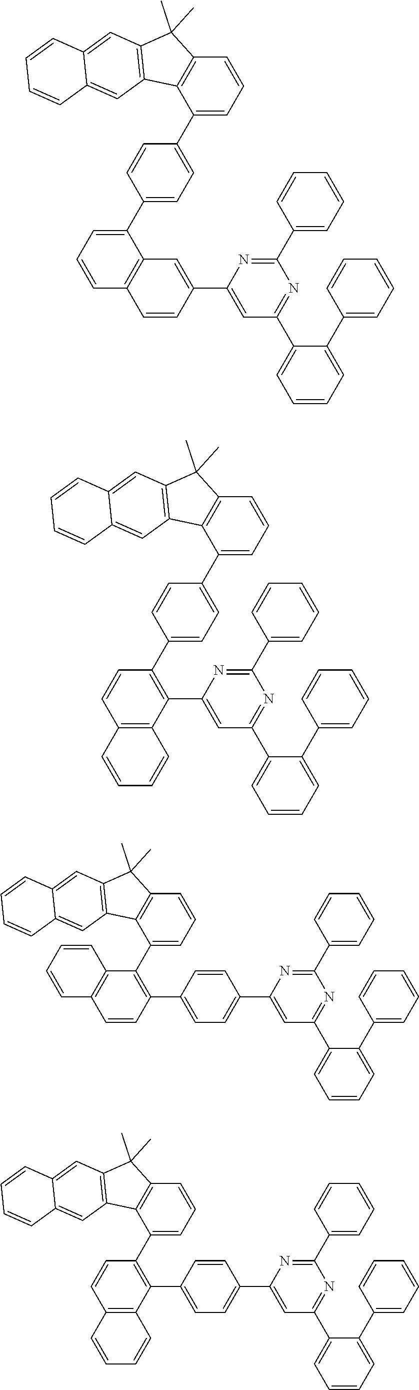 [SMILES: CC1(C)c2cc3ccccc3cc2-c2c(-c3ccc(-c4cccc5ccc(-c6cc(-c7ccccc7-c7ccccc7)nc(-c7ccccc7)n6)cc45)cc3)cccc21 ; CC1(C)c2cc3ccccc3cc2-c2c(-c3ccc(-c4ccc5ccccc5c4-c4cc(-c5ccccc5-c5ccccc5)nc(-c5ccccc5)n4)cc3)cccc21 ; CC1(C)c2cc3ccccc3cc2-c2c(-c3c(-c4ccc(-c5cc(-c6ccccc6-c6ccccc6)nc(-c6ccccc6)n5)cc4)ccc4ccccc34)cccc21 ; CC1(C)c2cc3ccccc3cc2-c2c(-c3ccc4ccccc4c3-c3ccc(-c4cc(-c5ccccc5-c5ccccc5)nc(-c5ccccc5)n4)cc3)cccc21]

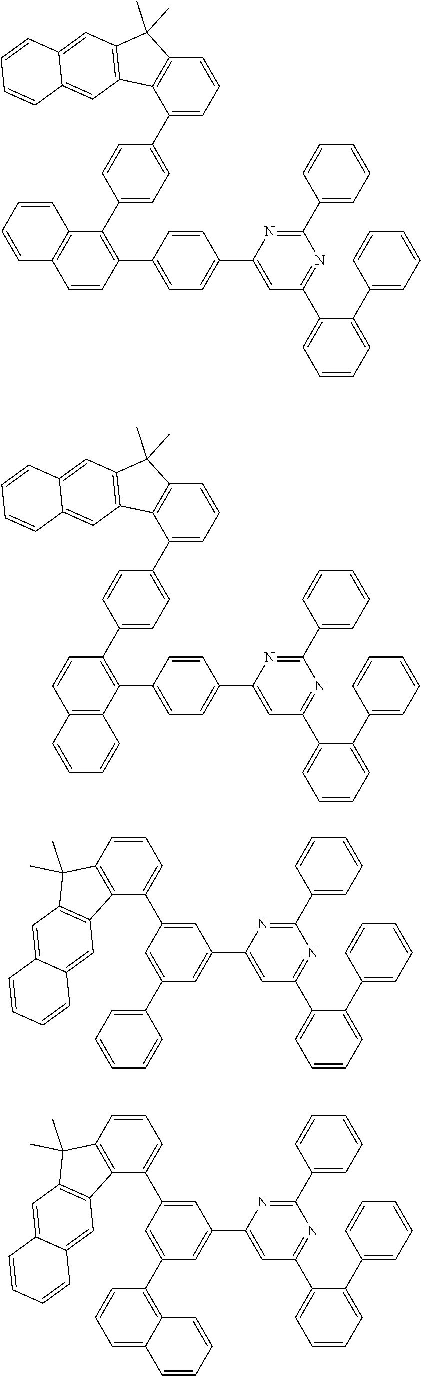

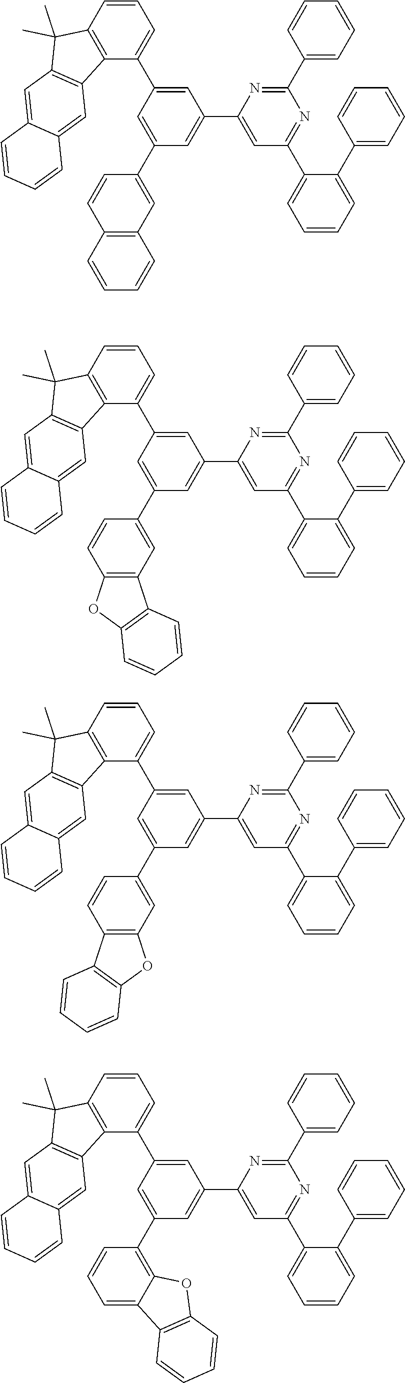

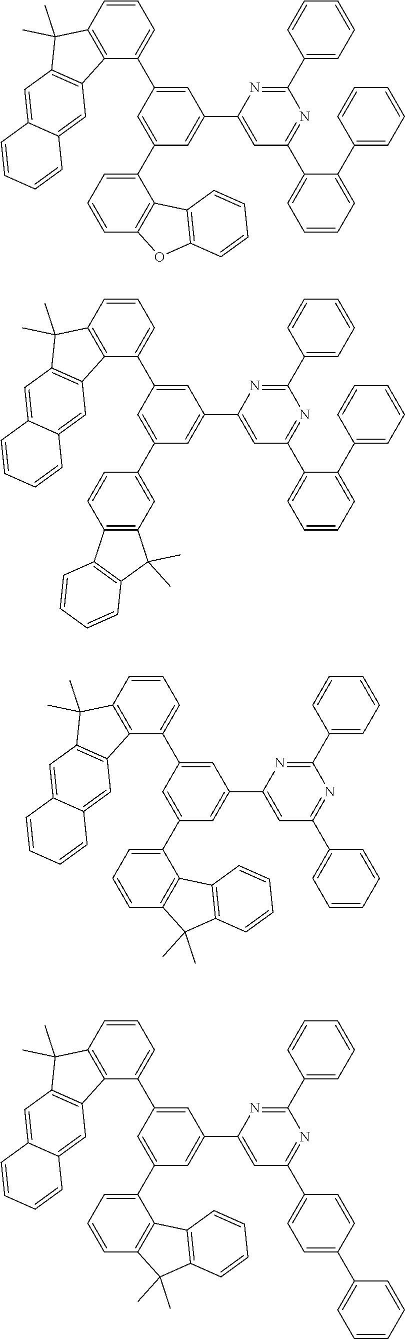 [SMILES: CC1(C)c2cc3ccccc3cc2-c2c(-c3cc(-c4cc(-c5ccccc5-c5ccccc5)nc(-c5ccccc5)n4)cc(-c4cccc5oc6ccccc6c45)c3)cccc21 ; CC1(C)c2ccccc2-c2ccc(-c3cc(-c4cc(-c5ccccc5-c5ccccc5)nc(-c5ccccc5)n4)cc(-c4cccc5c4-c4cc6ccccc6cc4C5(C)C)c3)cc21 ; CC1(C)c2ccccc2-c2c(-c3cc(-c4cc(-c5ccccc5)nc(-c5ccccc5)n4)cc(-c4cccc5c4-c4cc6ccccc6cc4C5(C)C)c3)cccc21 ; CC1(C)c2ccccc2-c2c(-c3cc(-c4cc(-c5ccc(-c6ccccc6)cc5)nc(-c5ccccc5)n4)cc(-c4cccc5c4-c4cc6ccccc6cc4C5(C)C)c3)cccc21]

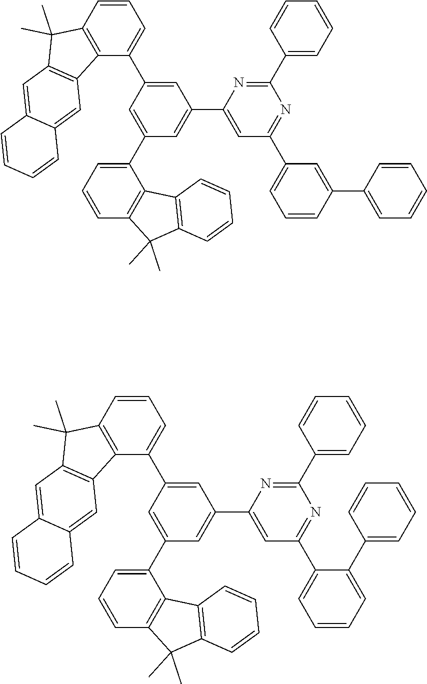 [SMILES: CC1(C)c2ccccc2-c2c(-c3cc(-c4cc(-c5cccc(-c6ccccc6)c5)nc(-c5ccccc5)n4)cc(-c4cccc5c4-c4cc6ccccc6cc4C5(C)C)c3)cccc21 ; CC1(C)c2ccccc2-c2c(-c3cc(-c4cc(-c5ccccc5-c5ccccc5)nc(-c5ccccc5)n4)cc(-c4cccc5c4-c4cc6ccccc6cc4C5(C)C)c3)cccc21]

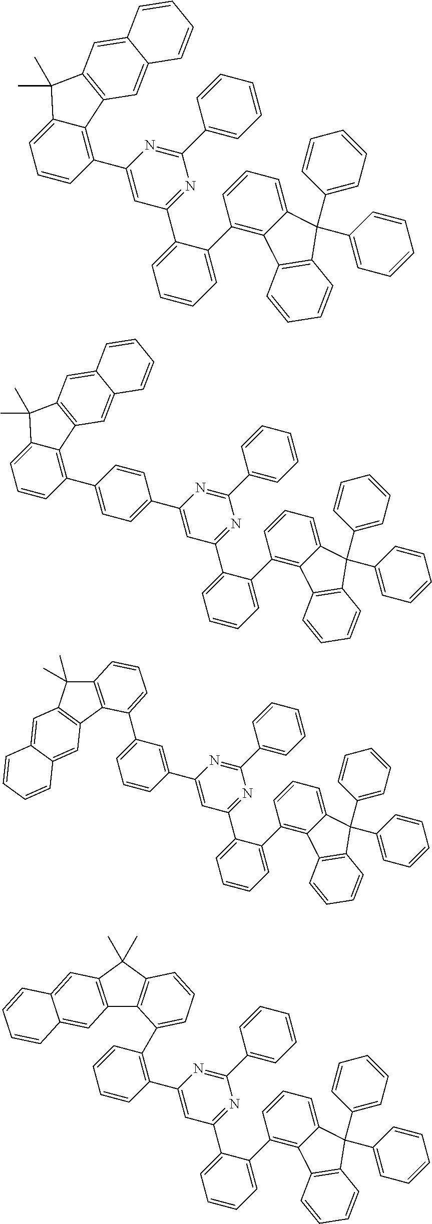 [SMILES: CC1(C)c2cc3ccccc3cc2-c2c(-c3cc(-c4ccccc4-c4cccc5c4-c4ccccc4C5(c4ccccc4)c4ccccc4)nc(-c4ccccc4)n3)cccc21 ; CC1(C)c2cc3ccccc3cc2-c2c(-c3ccc(-c4cc(-c5ccccc5-c5cccc6c5-c5ccccc5C6(c5ccccc5)c5ccccc5)nc(-c5ccccc5)n4)cc3)cccc21 ; CC1(C)c2cc3ccccc3cc2-c2c(-c3cccc(-c4cc(-c5ccccc5-c5cccc6c5-c5ccccc5C6(c5ccccc5)c5ccccc5)nc(-c5ccccc5)n4)c3)cccc21 ; CC1(C)c2cc3ccccc3cc2-c2c(-c3ccccc3-c3cc(-c4ccccc4-c4cccc5c4-c4ccccc4C5(c4ccccc4)c4ccccc4)nc(-c4ccccc4)n3)cccc21]

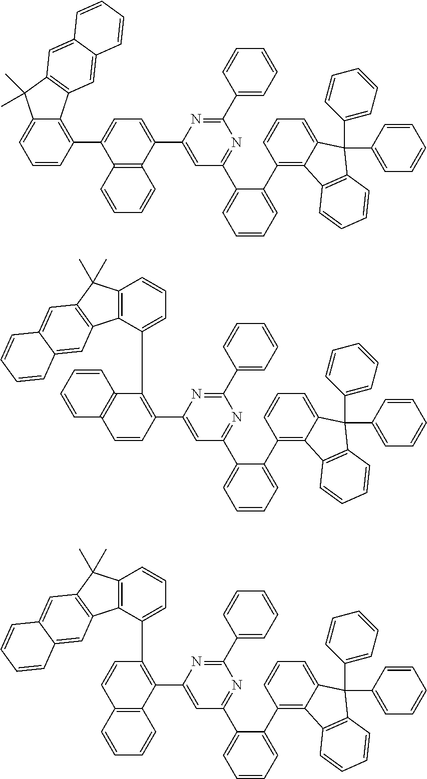 [SMILES: CC1(C)c2cc3ccccc3cc2-c2c(-c3ccc(-c4cc(-c5ccccc5-c5cccc6c5-c5ccccc5C6(c5ccccc5)c5ccccc5)nc(-c5ccccc5)n4)c4ccccc34)cccc21 ; CC1(C)c2cc3ccccc3cc2-c2c(-c3c(-c4cc(-c5ccccc5-c5cccc6c5-c5ccccc5C6(c5ccccc5)c5ccccc5)nc(-c5ccccc5)n4)ccc4ccccc34)cccc21 ; CC1(C)c2cc3ccccc3cc2-c2c(-c3ccc4ccccc4c3-c3cc(-c4ccccc4-c4cccc5c4-c4ccccc4C5(c4ccccc4)c4ccccc4)nc(-c4ccccc4)n3)cccc21]

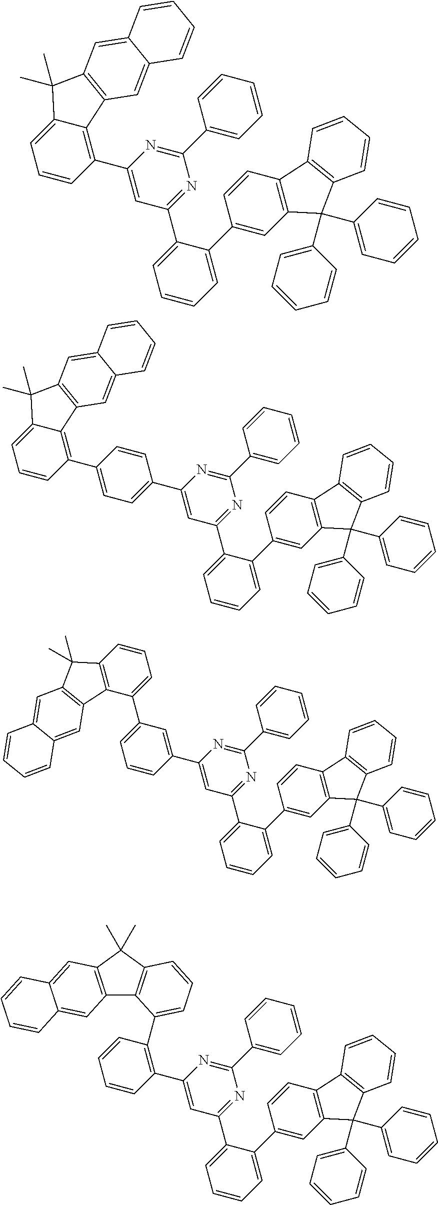

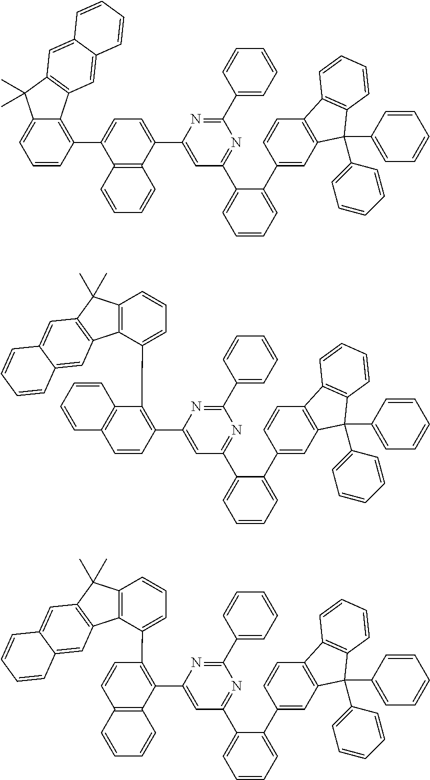 [SMILES: CC1(C)c2cc3ccccc3cc2-c2c(-c3ccc(-c4cc(-c5ccccc5-c5ccc6c(c5)C(c5ccccc5)(c5ccccc5)c5ccccc5-6)nc(-c5ccccc5)n4)c4ccccc34)cccc21 ; CC1(C)c2cc3ccccc3cc2-c2c(-c3c(-c4cc(-c5ccccc5-c5ccc6c(c5)C(c5ccccc5)(c5ccccc5)c5ccccc5-6)nc(-c5ccccc5)n4)ccc4ccccc34)cccc21 ; CC1(C)c2cc3ccccc3cc2-c2c(-c3ccc4ccccc4c3-c3cc(-c4ccccc4-c4ccc5c(c4)C(c4ccccc4)(c4ccccc4)c4ccccc4-5)nc(-c4ccccc4)n3)cccc21]

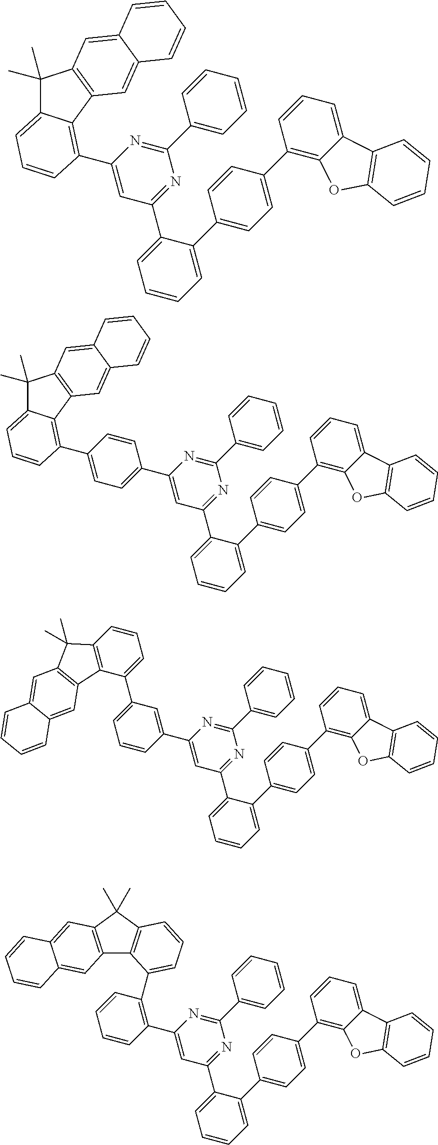

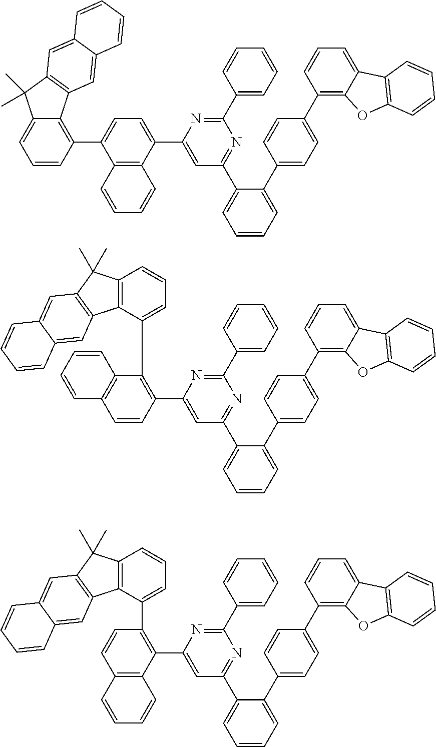

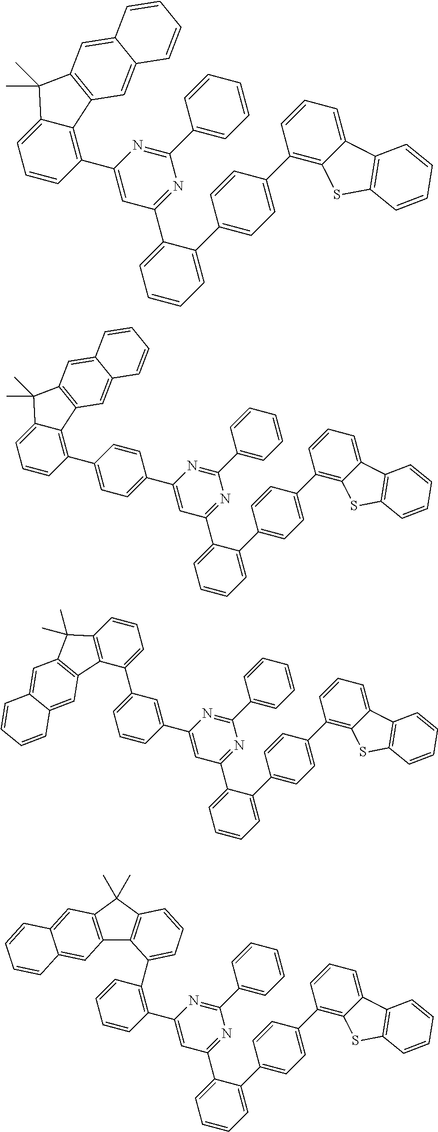

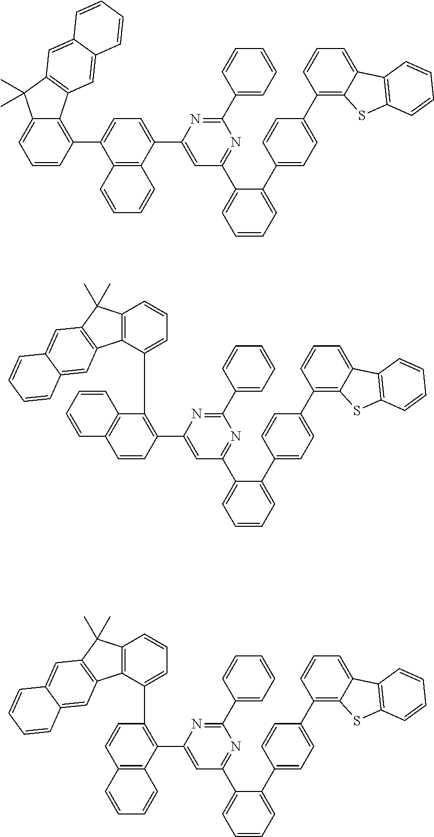 [SMILES: CC1(C)c2cc3ccccc3cc2-c2c(-c3ccc(-c4cc(-c5ccccc5-c5ccc(-c6cccc7c6sc6ccccc67)cc5)nc(-c5ccccc5)n4)c4ccccc34)cccc21 ; CC1(C)c2cc3ccccc3cc2-c2c(-c3c(-c4cc(-c5ccccc5-c5ccc(-c6cccc7c6sc6ccccc67)cc5)nc(-c5ccccc5)n4)ccc4ccccc34)cccc21 ; CC1(C)c2cc3ccccc3cc2-c2c(-c3ccc4ccccc4c3-c3cc(-c4ccccc4-c4ccc(-c5cccc6c5sc5ccccc56)cc4)nc(-c4ccccc4)n3)cccc21]

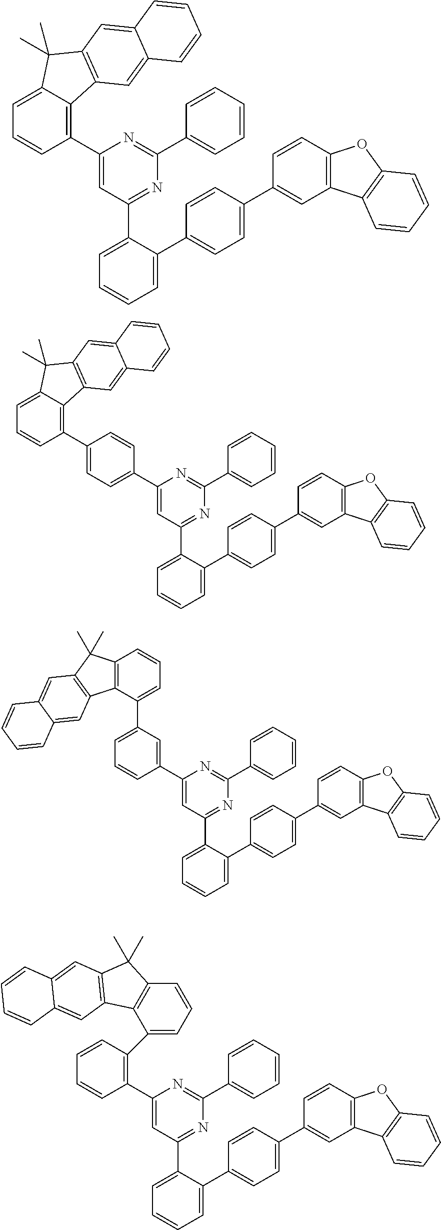

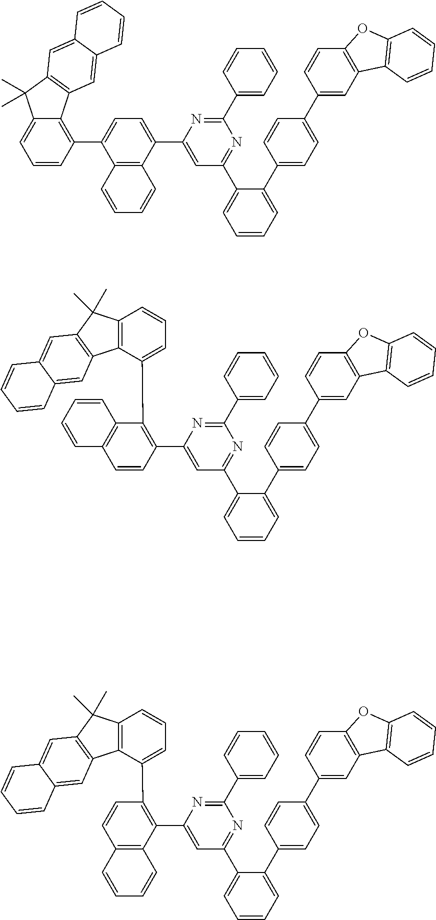 [SMILES: CC1(C)c2cc3ccccc3cc2-c2c(-c3ccc(-c4cc(-c5ccccc5-c5ccc(-c6ccc7oc8ccccc8c7c6)cc5)nc(-c5ccccc5)n4)c4ccccc34)cccc21 ; CC1(C)c2cc3ccccc3cc2-c2c(-c3c(-c4cc(-c5ccccc5-c5ccc(-c6ccc7oc8ccccc8c7c6)cc5)nc(-c5ccccc5)n4)ccc4ccccc34)cccc21 ; CC1(C)c2cc3ccccc3cc2-c2c(-c3ccc4ccccc4c3-c3cc(-c4ccccc4-c4ccc(-c5ccc6oc7ccccc7c6c5)cc4)nc(-c4ccccc4)n3)cccc21]

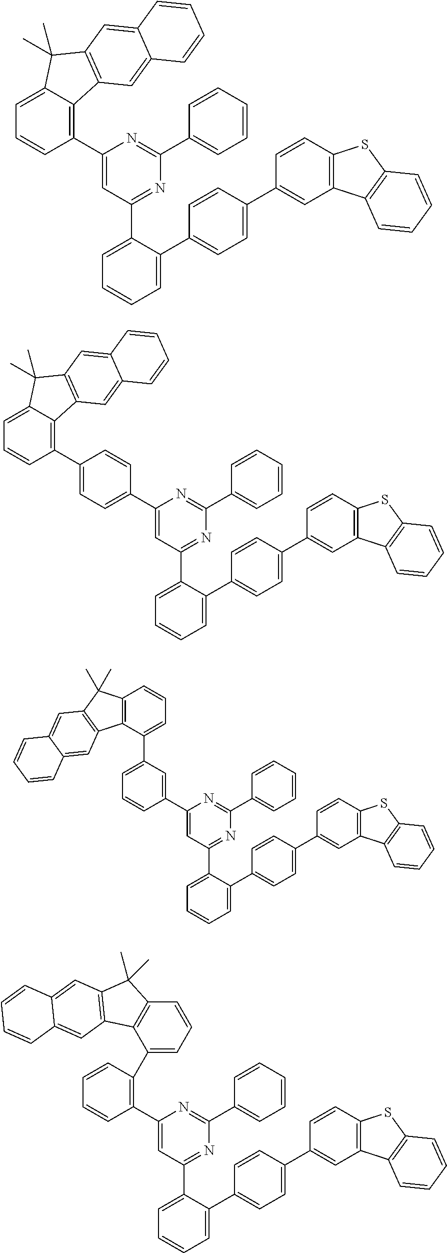

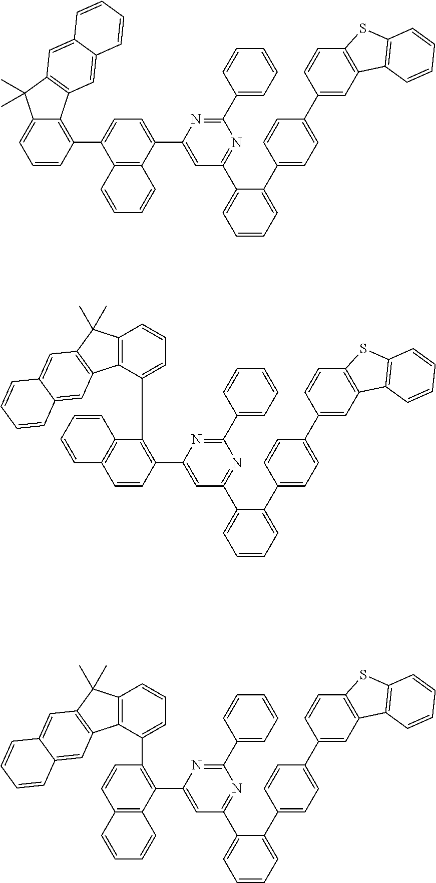 [SMILES: CC1(C)c2cc3ccccc3cc2-c2c(-c3ccc(-c4cc(-c5ccccc5-c5ccc(-c6ccc7sc8ccccc8c7c6)cc5)nc(-c5ccccc5)n4)c4ccccc34)cccc21 ; CC1(C)c2cc3ccccc3cc2-c2c(-c3c(-c4cc(-c5ccccc5-c5ccc(-c6ccc7sc8ccccc8c7c6)cc5)nc(-c5ccccc5)n4)ccc4ccccc34)cccc21 ; CC1(C)c2cc3ccccc3cc2-c2c(-c3ccc4ccccc4c3-c3cc(-c4ccccc4-c4ccc(-c5ccc6sc7ccccc7c6c5)cc4)nc(-c4ccccc4)n3)cccc21]

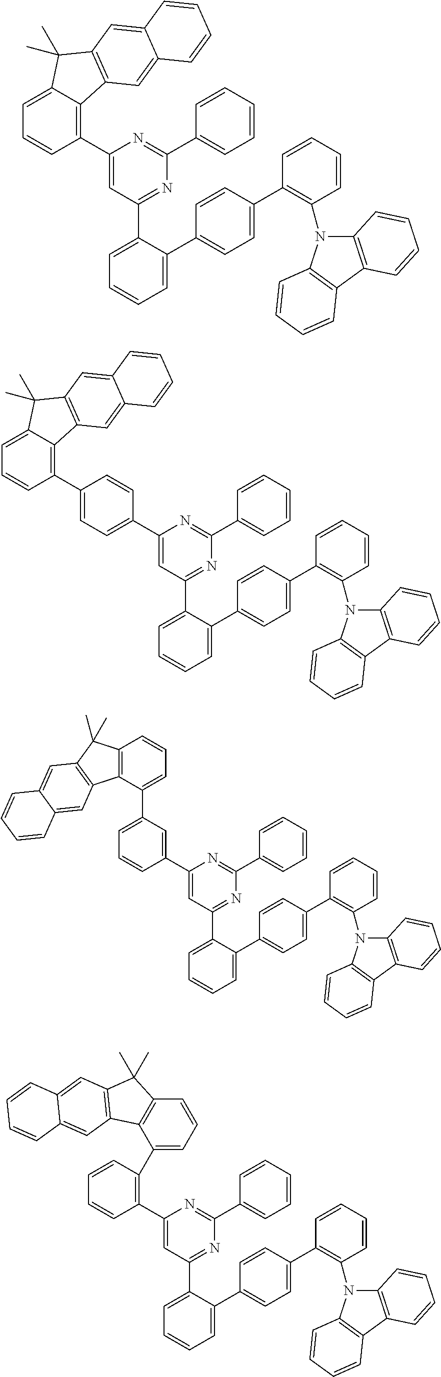 [SMILES: CC1(C)c2cc3ccccc3cc2-c2c(-c3cc(-c4ccccc4-c4ccc(-c5ccccc5-n5c6ccccc6c6ccccc65)cc4)nc(-c4ccccc4)n3)cccc21 ; CC1(C)c2cc3ccccc3cc2-c2c(-c3ccc(-c4cc(-c5ccccc5-c5ccc(-c6ccccc6-n6c7ccccc7c7ccccc76)cc5)nc(-c5ccccc5)n4)cc3)cccc21 ; CC1(C)c2cc3ccccc3cc2-c2c(-c3cccc(-c4cc(-c5ccccc5-c5ccc(-c6ccccc6-n6c7ccccc7c7ccccc76)cc5)nc(-c5ccccc5)n4)c3)cccc21 ; CC1(C)c2cc3ccccc3cc2-c2c(-c3ccccc3-c3cc(-c4ccccc4-c4ccc(-c5ccccc5-n5c6ccccc6c6ccccc65)cc4)nc(-c4ccccc4)n3)cccc21]

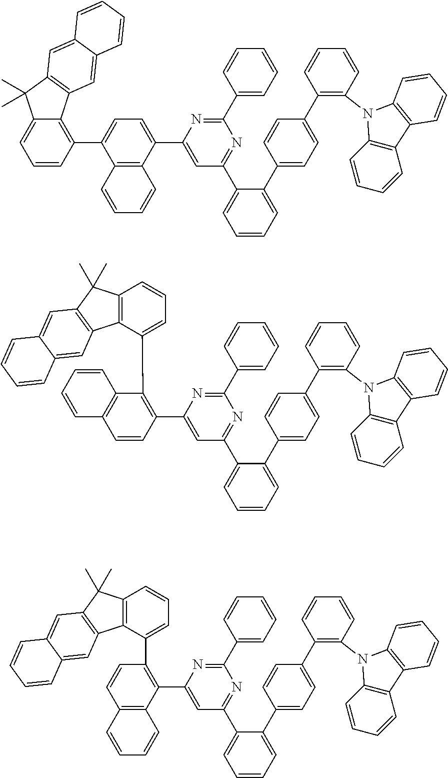 [SMILES: CC1(C)c2cc3ccccc3cc2-c2c(-c3ccc(-c4cc(-c5ccccc5-c5ccc(-c6ccccc6-n6c7ccccc7c7ccccc76)cc5)nc(-c5ccccc5)n4)c4ccccc34)cccc21 ; CC1(C)c2cc3ccccc3cc2-c2c(-c3c(-c4cc(-c5ccccc5-c5ccc(-c6ccccc6-n6c7ccccc7c7ccccc76)cc5)nc(-c5ccccc5)n4)ccc4ccccc34)cccc21 ; CC1(C)c2cc3ccccc3cc2-c2c(-c3ccc4ccccc4c3-c3cc(-c4ccccc4-c4ccc(-c5ccccc5-n5c6ccccc6c6ccccc65)cc4)nc(-c4ccccc4)n3)cccc21]

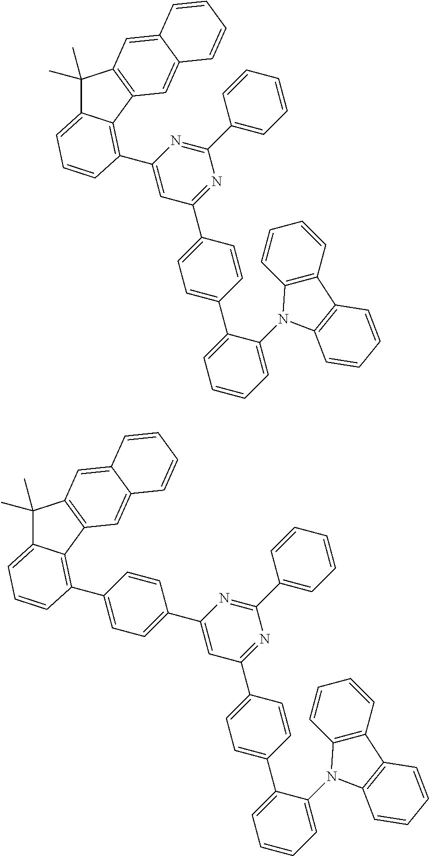 [SMILES: CC1(C)c2cc3ccccc3cc2-c2c(-c3cc(-c4ccc(-c5ccccc5-n5c6ccccc6c6ccccc65)cc4)nc(-c4ccccc4)n3)cccc21 ; CC1(C)c2cc3ccccc3cc2-c2c(-c3ccc(-c4cc(-c5ccc(-c6ccccc6-n6c7ccccc7c7ccccc76)cc5)nc(-c5ccccc5)n4)cc3)cccc21]

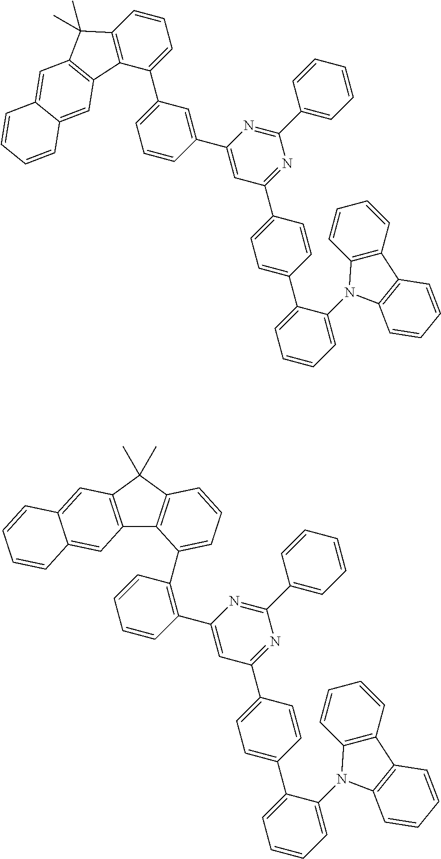 [SMILES: CC1(C)c2cc3ccccc3cc2-c2c(-c3cccc(-c4cc(-c5ccc(-c6ccccc6-n6c7ccccc7c7ccccc76)cc5)nc(-c5ccccc5)n4)c3)cccc21 ; CC1(C)c2cc3ccccc3cc2-c2c(-c3ccccc3-c3cc(-c4ccc(-c5ccccc5-n5c6ccccc6c6ccccc65)cc4)nc(-c4ccccc4)n3)cccc21]

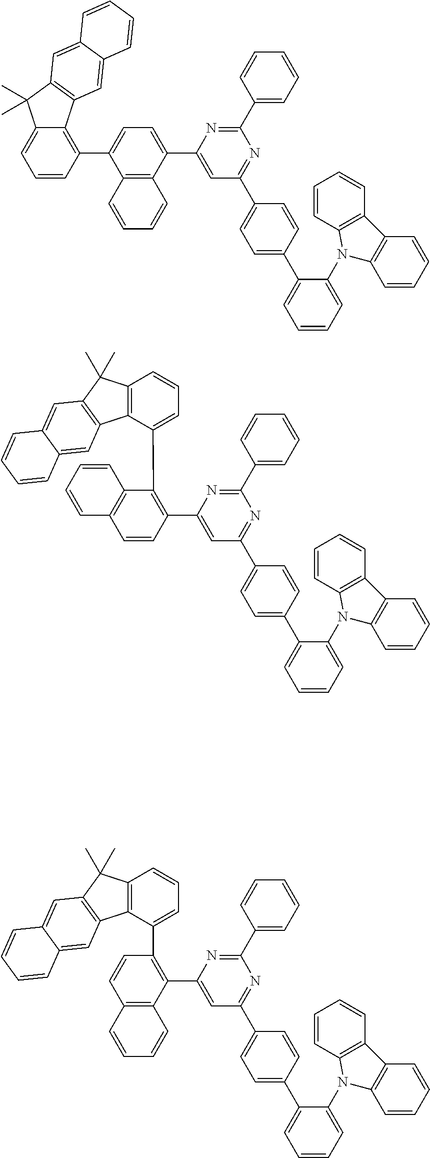 [SMILES: CC1(C)c2cc3ccccc3cc2-c2c(-c3ccc(-c4cc(-c5ccc(-c6ccccc6-n6c7ccccc7c7ccccc76)cc5)nc(-c5ccccc5)n4)c4ccccc34)cccc21 ; CC1(C)c2cc3ccccc3cc2-c2c(-c3c(-c4cc(-c5ccc(-c6ccccc6-n6c7ccccc7c7ccccc76)cc5)nc(-c5ccccc5)n4)ccc4ccccc34)cccc21 ; CC1(C)c2cc3ccccc3cc2-c2c(-c3ccc4ccccc4c3-c3cc(-c4ccc(-c5ccccc5-n5c6ccccc6c6ccccc65)cc4)nc(-c4ccccc4)n3)cccc21]

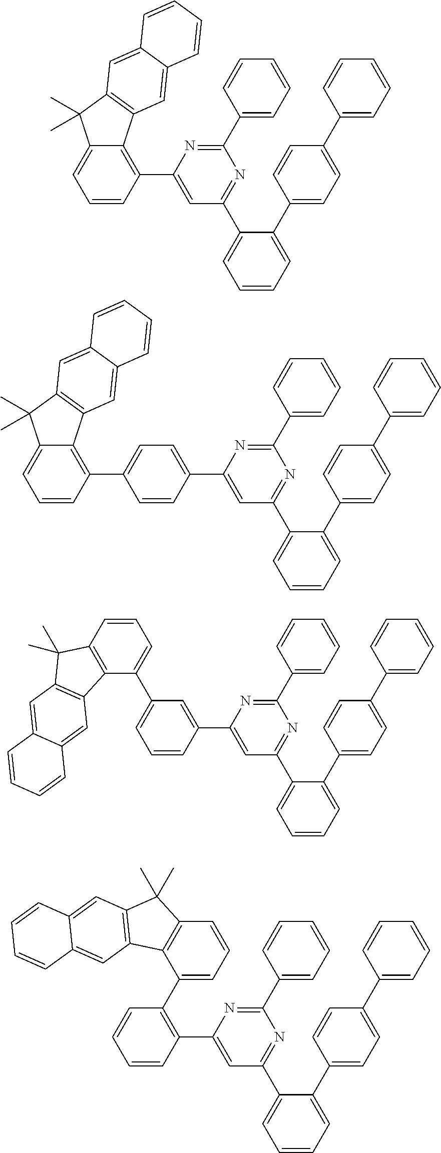

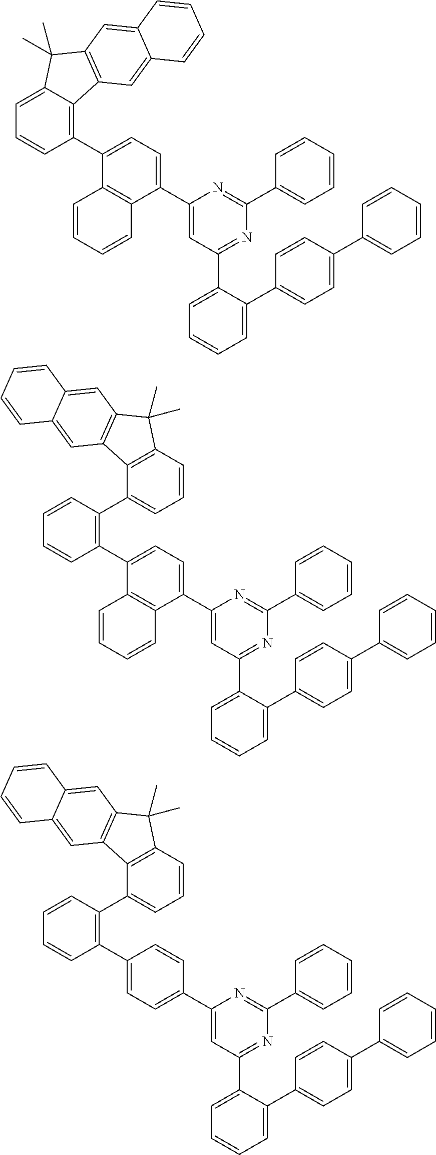 [SMILES: CC1(C)c2cc3ccccc3cc2-c2c(-c3ccc(-c4cc(-c5ccccc5-c5ccc(-c6ccccc6)cc5)nc(-c5ccccc5)n4)c4ccccc34)cccc21 ; CC1(C)c2cc3ccccc3cc2-c2c(-c3ccccc3-c3ccc(-c4cc(-c5ccccc5-c5ccc(-c6ccccc6)cc5)nc(-c5ccccc5)n4)c4ccccc34)cccc21 ; CC1(C)c2cc3ccccc3cc2-c2c(-c3ccccc3-c3ccc(-c4cc(-c5ccccc5-c5ccc(-c6ccccc6)cc5)nc(-c5ccccc5)n4)cc3)cccc21]

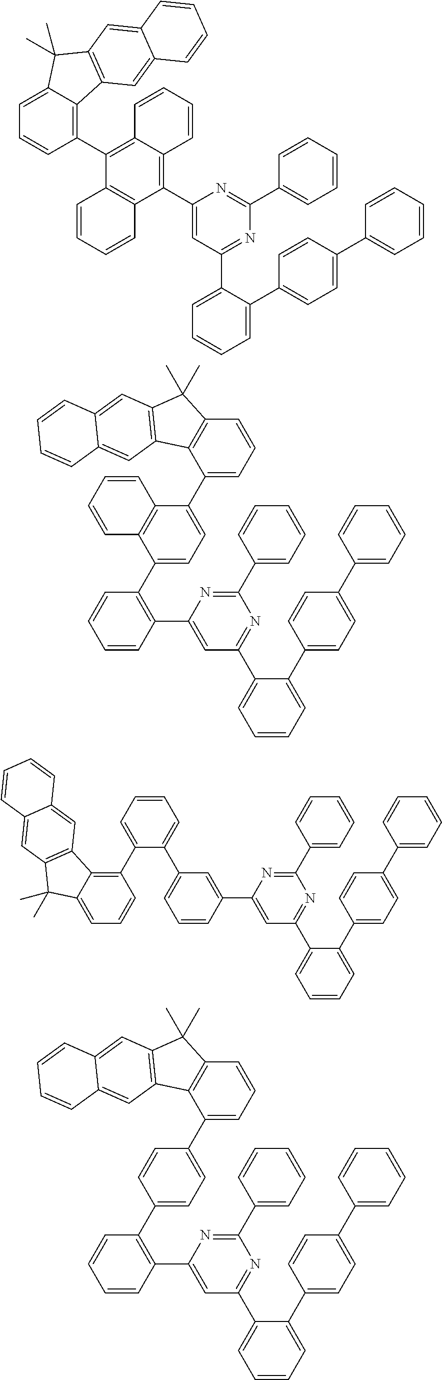

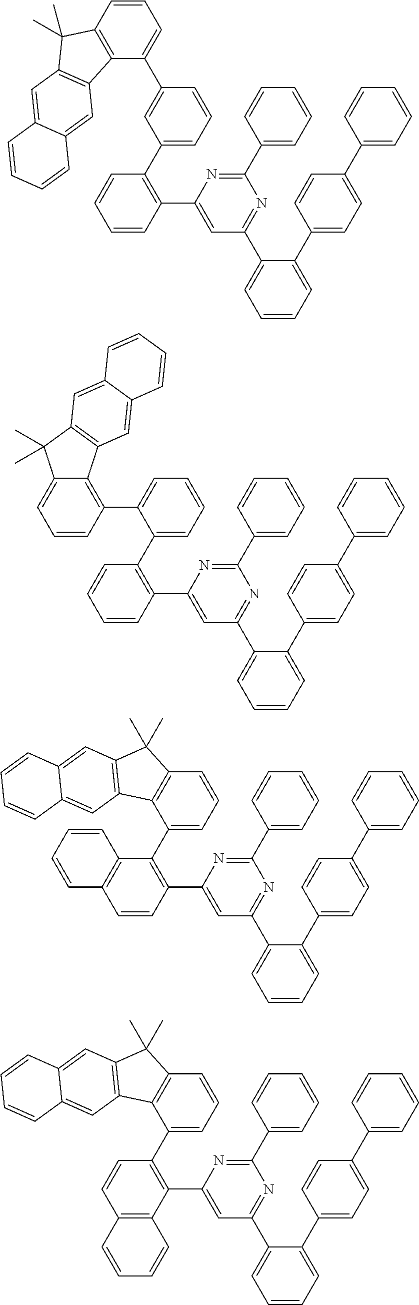 [SMILES: CC1(C)c2cc3ccccc3cc2-c2c(-c3cccc(-c4ccccc4-c4cc(-c5ccccc5-c5ccc(-c6ccccc6)cc5)nc(-c5ccccc5)n4)c3)cccc21 ; CC1(C)c2cc3ccccc3cc2-c2c(-c3ccccc3-c3ccccc3-c3cc(-c4ccccc4-c4ccc(-c5ccccc5)cc4)nc(-c4ccccc4)n3)cccc21 ; CC1(C)c2cc3ccccc3cc2-c2c(-c3c(-c4cc(-c5ccccc5-c5ccc(-c6ccccc6)cc5)nc(-c5ccccc5)n4)ccc4ccccc34)cccc21 ; CC1(C)c2cc3ccccc3cc2-c2c(-c3ccc4ccccc4c3-c3cc(-c4ccccc4-c4ccc(-c5ccccc5)cc4)nc(-c4ccccc4)n3)cccc21]

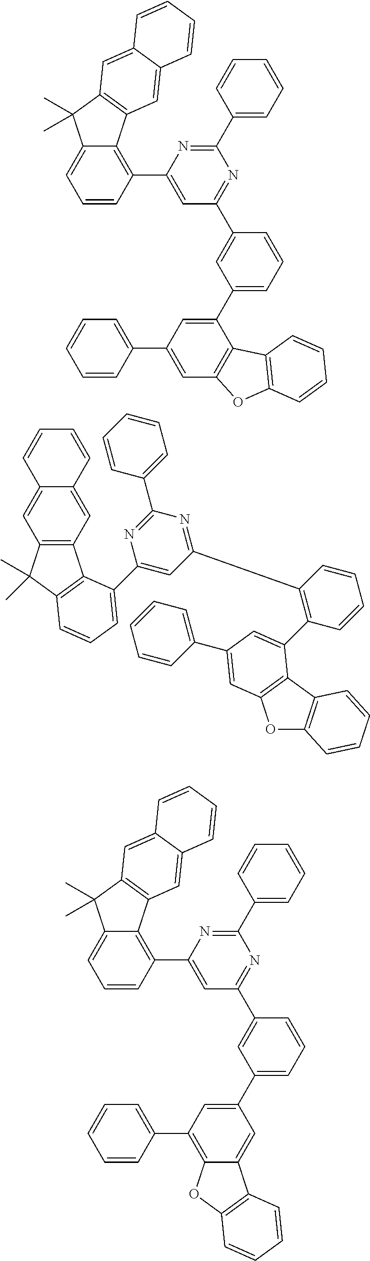

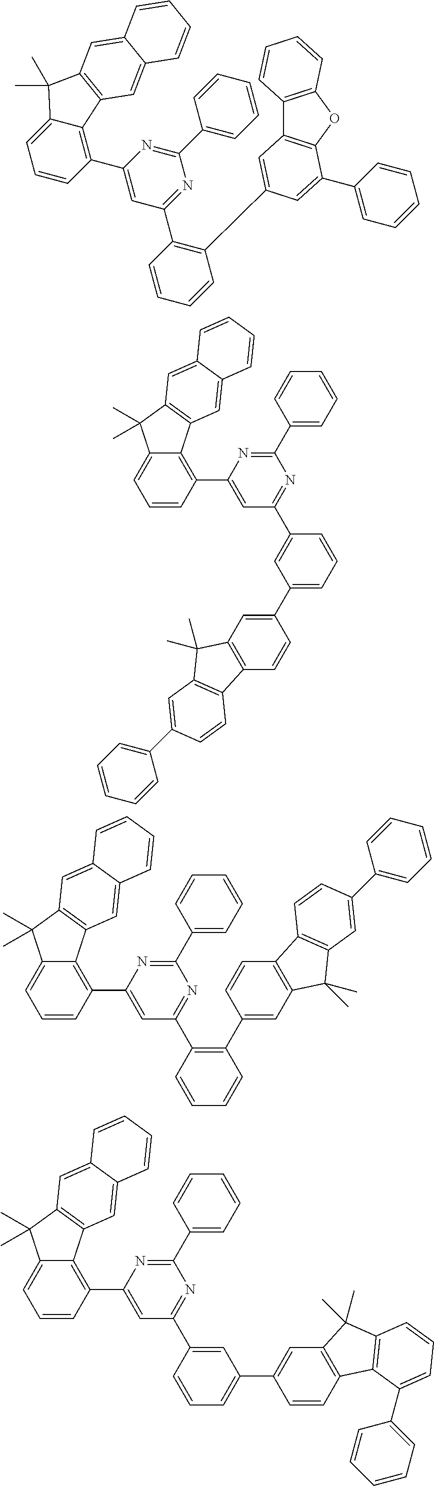

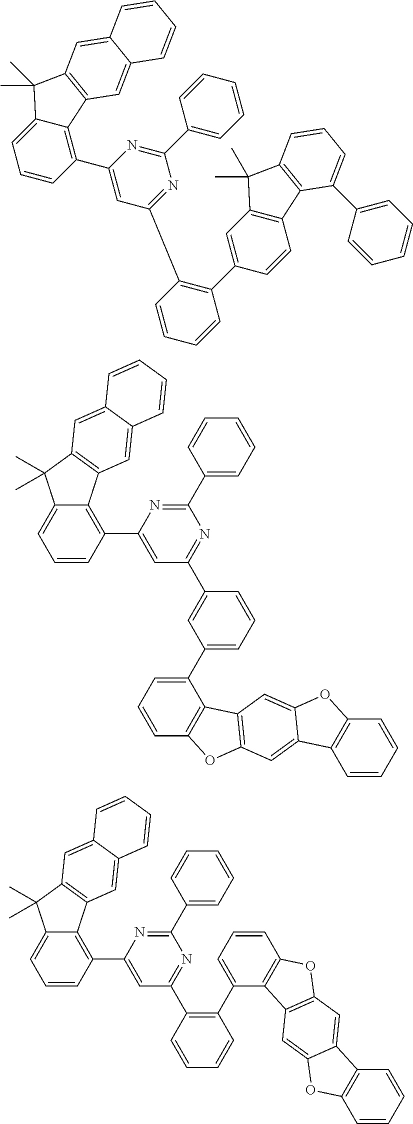

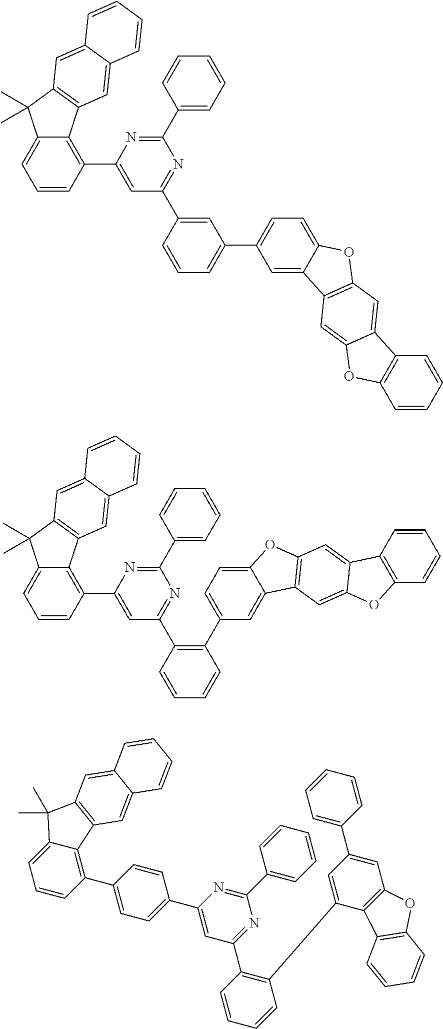 [SMILES: CC1(C)c2cc3ccccc3cc2-c2c(-c3cc(-c4cccc(-c5ccc6oc7cc8c(cc7c6c5)oc5ccccc58)c4)nc(-c4ccccc4)n3)cccc21 ; CC1(C)c2cc3ccccc3cc2-c2c(-c3cc(-c4ccccc4-c4ccc5oc6cc7c(cc6c5c4)oc4ccccc47)nc(-c4ccccc4)n3)cccc21 ; CC1(C)c2cc3ccccc3cc2-c2c(-c3ccc(-c4cc(-c5ccccc5-c5cc(-c6ccccc6)cc6oc7ccccc7c56)nc(-c5ccccc5)n4)cc3)cccc21]

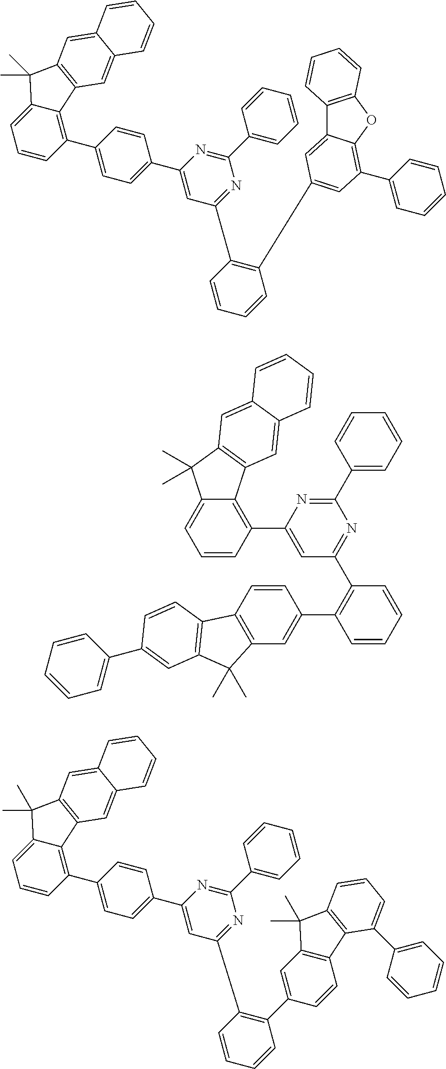 [SMILES: CC1(C)c2cc3ccccc3cc2-c2c(-c3ccc(-c4cc(-c5ccccc5-c5cc(-c6ccccc6)c6oc7ccccc7c6c5)nc(-c5ccccc5)n4)cc3)cccc21 ; CC1(C)c2cc(-c3ccccc3)ccc2-c2ccc(-c3ccccc3-c3cc(-c4cccc5c4-c4cc6ccccc6cc4C5(C)C)nc(-c4ccccc4)n3)cc21 ; CC1(C)c2cc(-c3ccccc3-c3cc(-c4ccc(-c5cccc6c5-c5cc7ccccc7cc5C6(C)C)cc4)nc(-c4ccccc4)n3)ccc2-c2c(-c3ccccc3)cccc21]

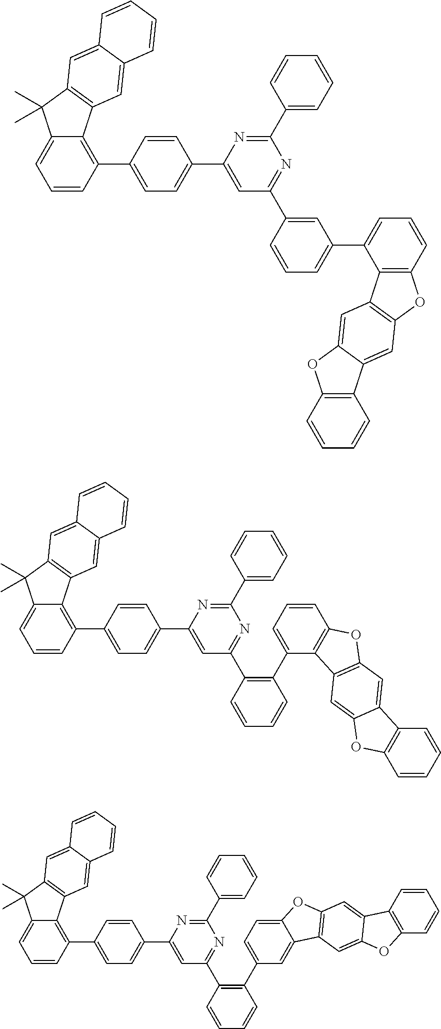 [SMILES: CC1(C)c2cc3ccccc3cc2-c2c(-c3ccc(-c4cc(-c5cccc(-c6cccc7oc8cc9c(cc8c67)oc6ccccc69)c5)nc(-c5ccccc5)n4)cc3)cccc21 ; CC1(C)c2cc3ccccc3cc2-c2c(-c3ccc(-c4cc(-c5ccccc5-c5cccc6oc7cc8c(cc7c56)oc5ccccc58)nc(-c5ccccc5)n4)cc3)cccc21 ; CC1(C)c2cc3ccccc3cc2-c2c(-c3ccc(-c4cc(-c5ccccc5-c5ccc6oc7cc8c(cc7c6c5)oc5ccccc58)nc(-c5ccccc5)n4)cc3)cccc21]

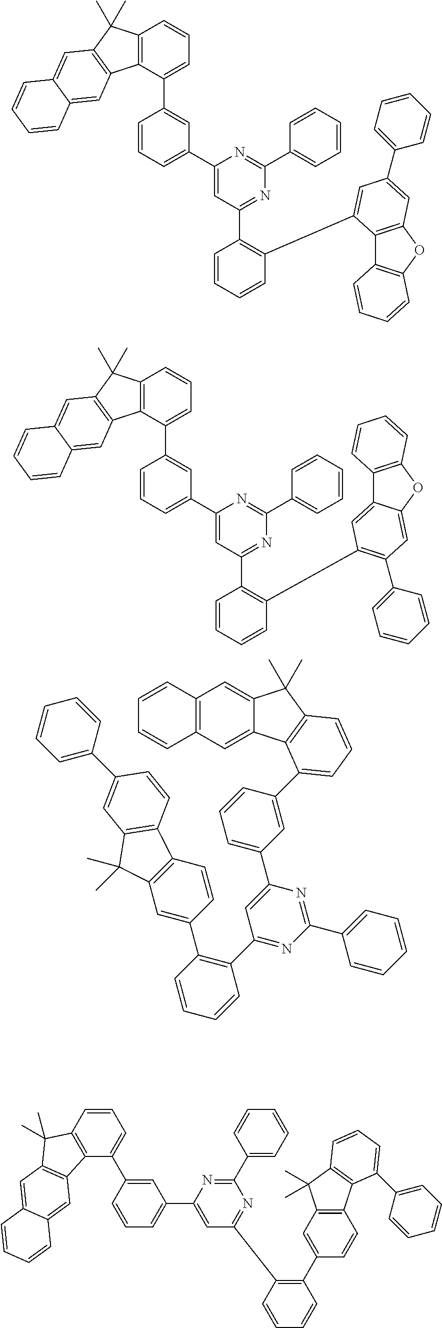

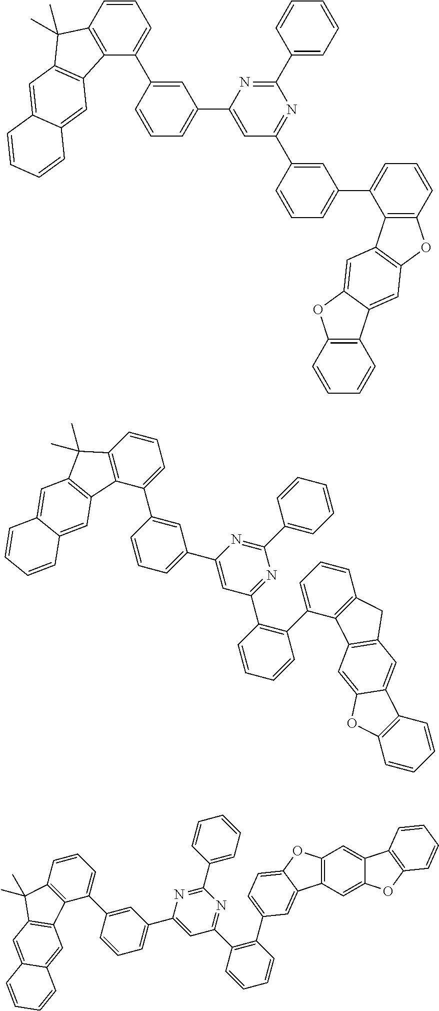 [SMILES: CC1(C)c2cc3ccccc3cc2-c2c(-c3cccc(-c4cc(-c5cccc(-c6cccc7oc8cc9c(cc8c67)oc6ccccc69)c5)nc(-c5ccccc5)n4)c3)cccc21 ; CC1(C)c2cc3ccccc3cc2-c2c(-c3cccc(-c4cc(-c5ccccc5-c5cccc6c5-c5cc7oc8ccccc8c7cc5C6)nc(-c5ccccc5)n4)c3)cccc21 ; CC1(C)c2cc3ccccc3cc2-c2c(-c3cccc(-c4cc(-c5ccccc5-c5ccc6oc7cc8c(cc7c6c5)oc5ccccc58)nc(-c5ccccc5)n4)c3)cccc21]

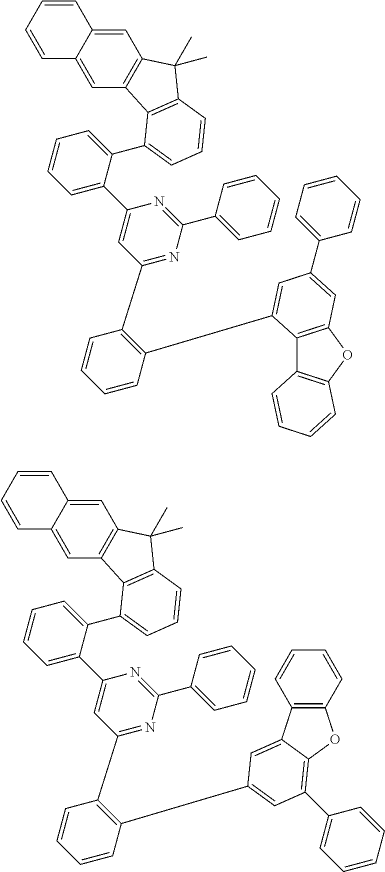 [SMILES: CC1(C)c2cc3ccccc3cc2-c2c(-c3ccccc3-c3cc(-c4ccccc4-c4cc(-c5ccccc5)cc5oc6ccccc6c45)nc(-c4ccccc4)n3)cccc21 ; CC1(C)c2cc3ccccc3cc2-c2c(-c3ccccc3-c3cc(-c4ccccc4-c4cc(-c5ccccc5)c5oc6ccccc6c5c4)nc(-c4ccccc4)n3)cccc21]

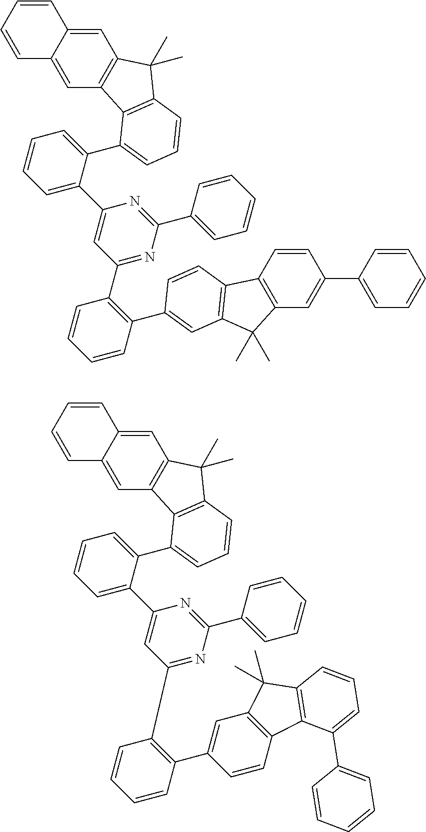

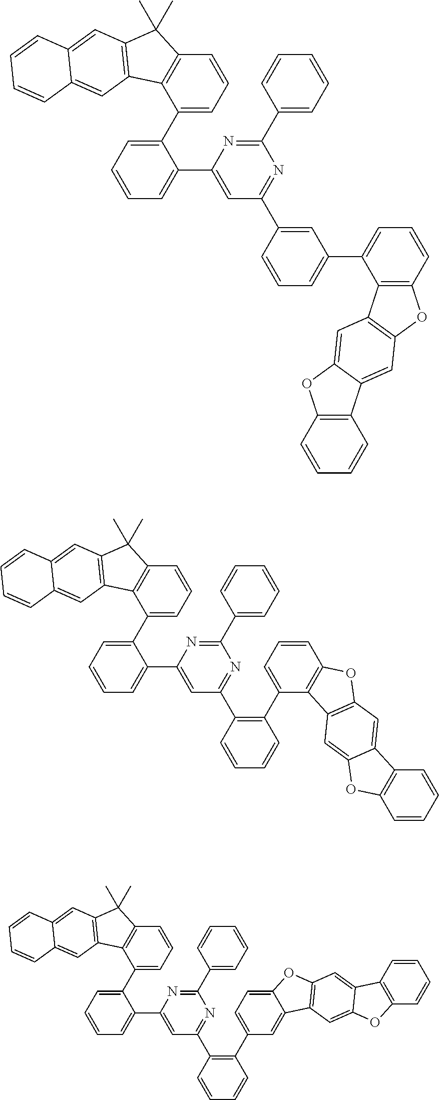 [SMILES: CC1(C)c2cc3ccccc3cc2-c2c(-c3ccccc3-c3cc(-c4cccc(-c5cccc6oc7cc8c(cc7c56)oc5ccccc58)c4)nc(-c4ccccc4)n3)cccc21 ; CC1(C)c2cc3ccccc3cc2-c2c(-c3ccccc3-c3cc(-c4ccccc4-c4cccc5oc6cc7c(cc6c45)oc4ccccc47)nc(-c4ccccc4)n3)cccc21 ; CC1(C)c2cc3ccccc3cc2-c2c(-c3ccccc3-c3cc(-c4ccccc4-c4ccc5oc6cc7c(cc6c5c4)oc4ccccc47)nc(-c4ccccc4)n3)cccc21]

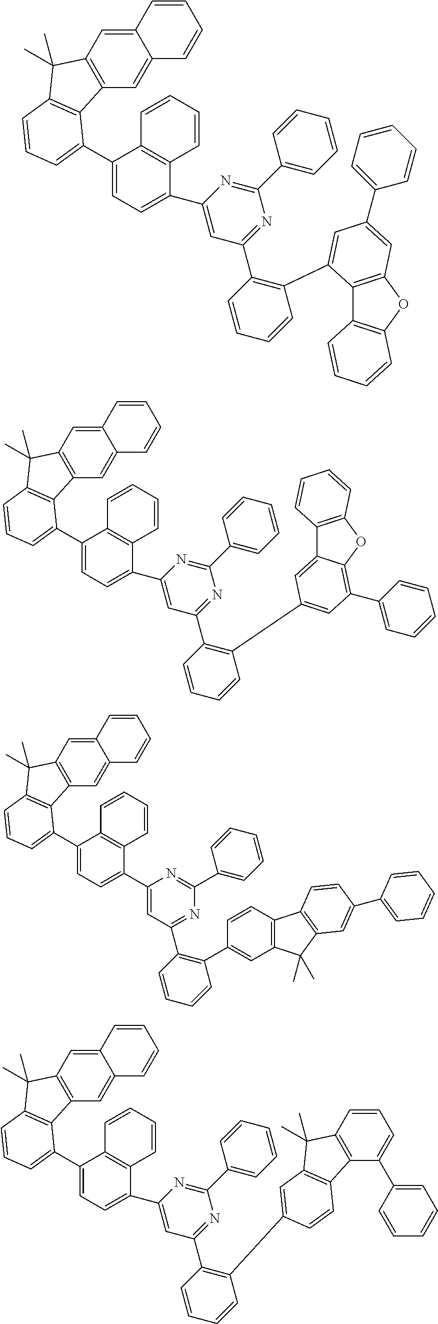

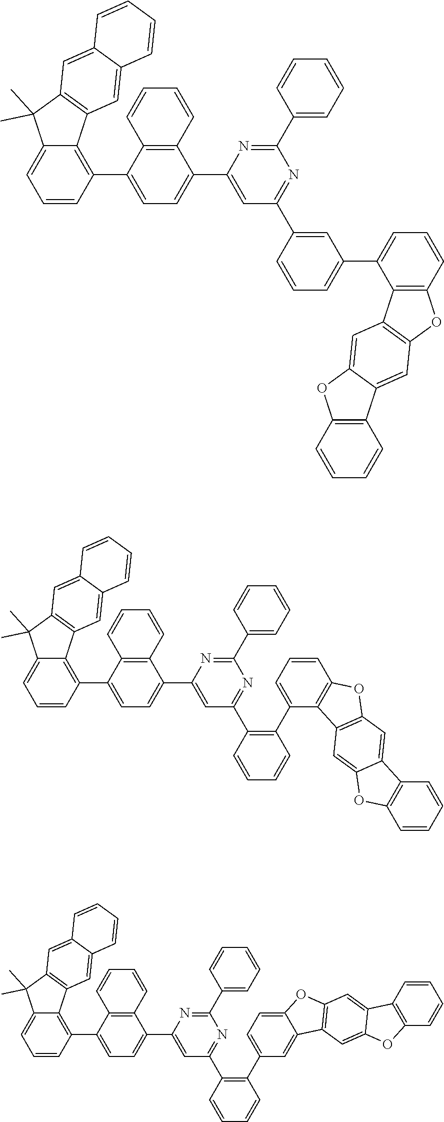 [SMILES: CC1(C)c2cc3ccccc3cc2-c2c(-c3ccc(-c4cc(-c5cccc(-c6cccc7oc8cc9c(cc8c67)oc6ccccc69)c5)nc(-c5ccccc5)n4)c4ccccc34)cccc21 ; CC1(C)c2cc3ccccc3cc2-c2c(-c3ccc(-c4cc(-c5ccccc5-c5cccc6oc7cc8c(cc7c56)oc5ccccc58)nc(-c5ccccc5)n4)c4ccccc34)cccc21 ; CC1(C)c2cc3ccccc3cc2-c2c(-c3ccc(-c4cc(-c5ccccc5-c5ccc6oc7cc8c(cc7c6c5)oc5ccccc58)nc(-c5ccccc5)n4)c4ccccc34)cccc21]

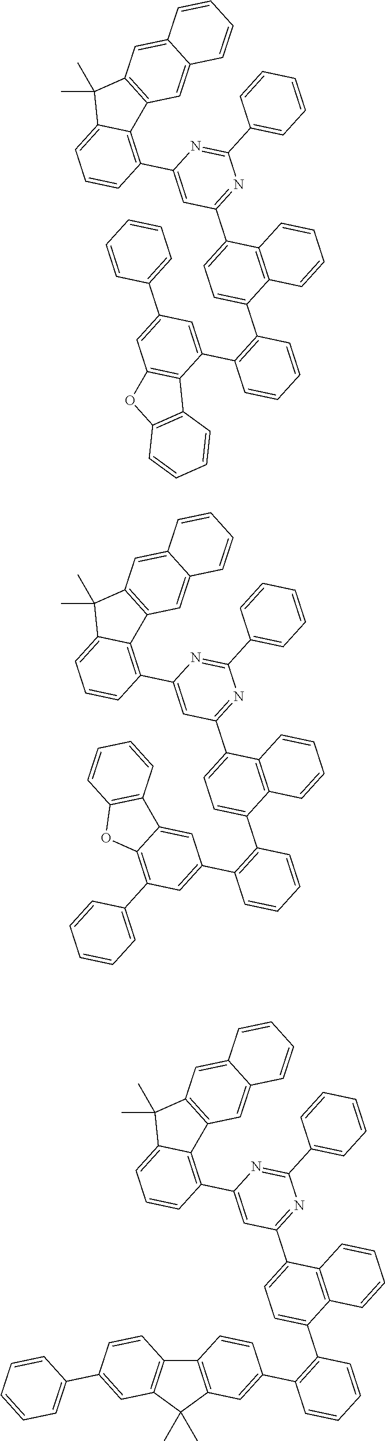

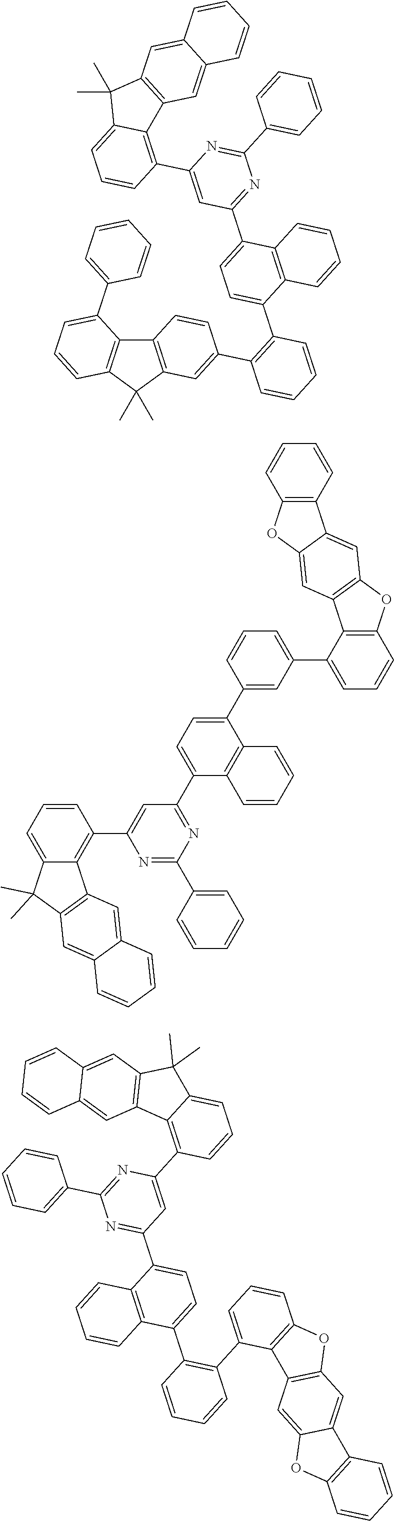

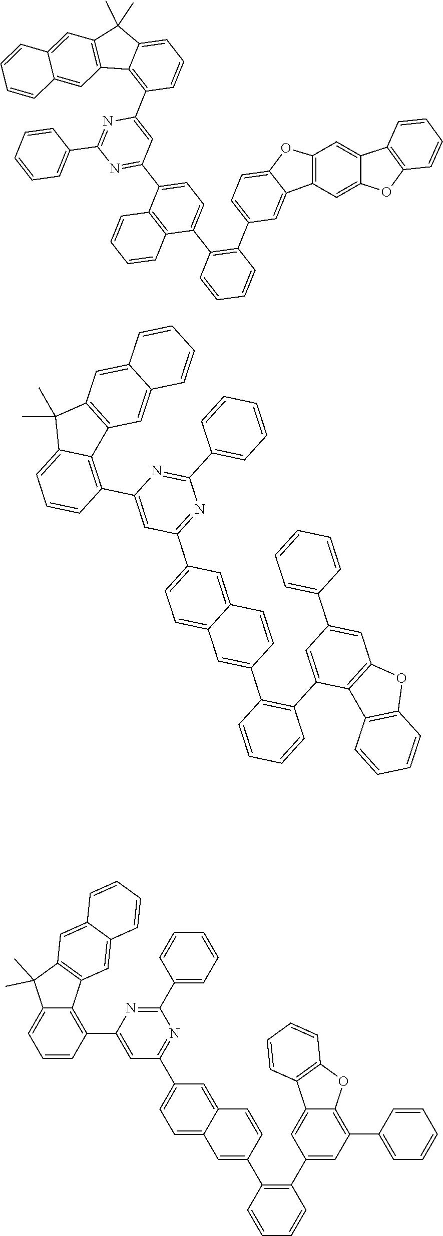 [SMILES: CC1(C)c2cc3ccccc3cc2-c2c(-c3cc(-c4ccc(-c5ccccc5-c5ccc6oc7cc8c(cc7c6c5)oc5ccccc58)c5ccccc45)nc(-c4ccccc4)n3)cccc21 ; CC1(C)c2cc3ccccc3cc2-c2c(-c3cc(-c4ccc5cc(-c6ccccc6-c6cc(-c7ccccc7)cc7oc8ccccc8c67)ccc5c4)nc(-c4ccccc4)n3)cccc21 ; CC1(C)c2cc3ccccc3cc2-c2c(-c3cc(-c4ccc5cc(-c6ccccc6-c6cc(-c7ccccc7)c7oc8ccccc8c7c6)ccc5c4)nc(-c4ccccc4)n3)cccc21]

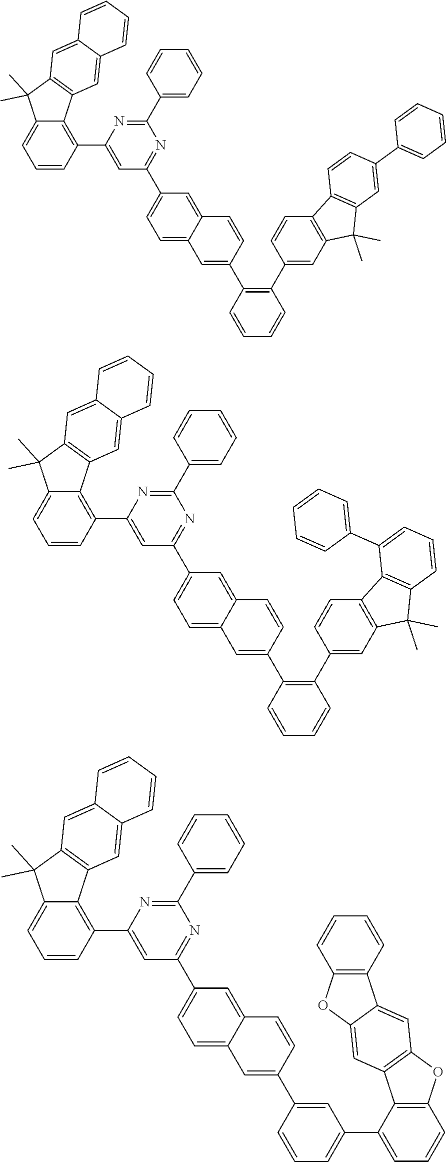

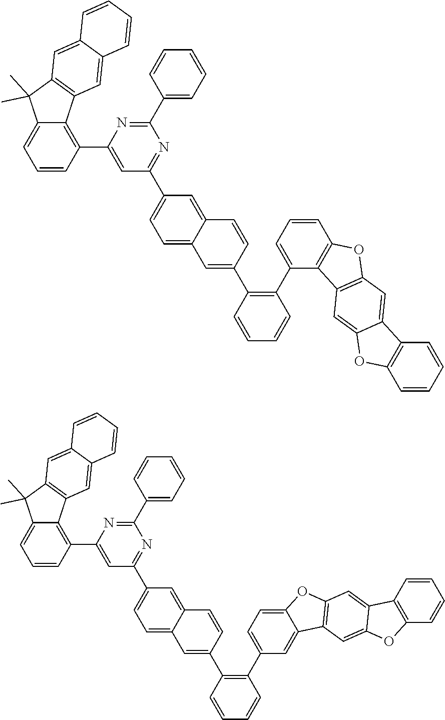 [SMILES: CC1(C)c2cc3ccccc3cc2-c2c(-c3cc(-c4ccc5cc(-c6ccccc6-c6cccc7oc8cc9c(cc8c67)oc6ccccc69)ccc5c4)nc(-c4ccccc4)n3)cccc21 ; CC1(C)c2cc3ccccc3cc2-c2c(-c3cc(-c4ccc5cc(-c6ccccc6-c6ccc7oc8cc9c(cc8c7c6)oc6ccccc69)ccc5c4)nc(-c4ccccc4)n3)cccc21]

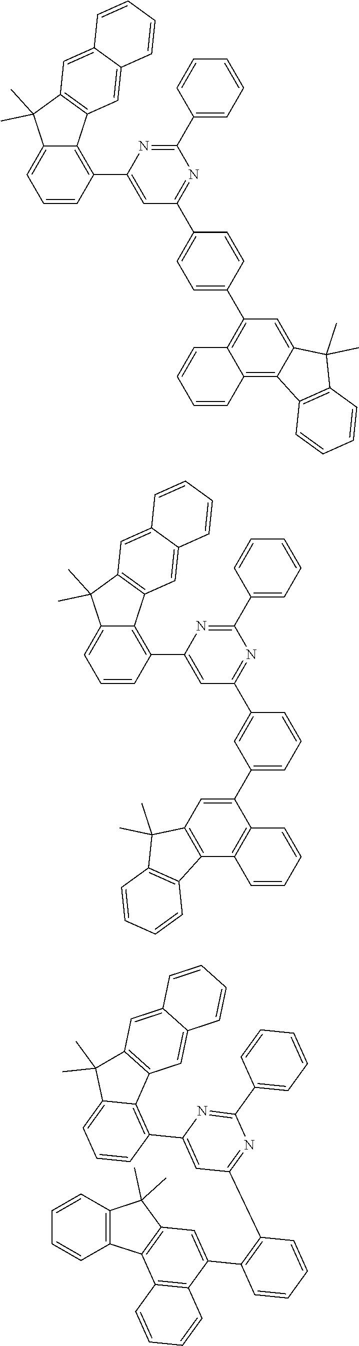

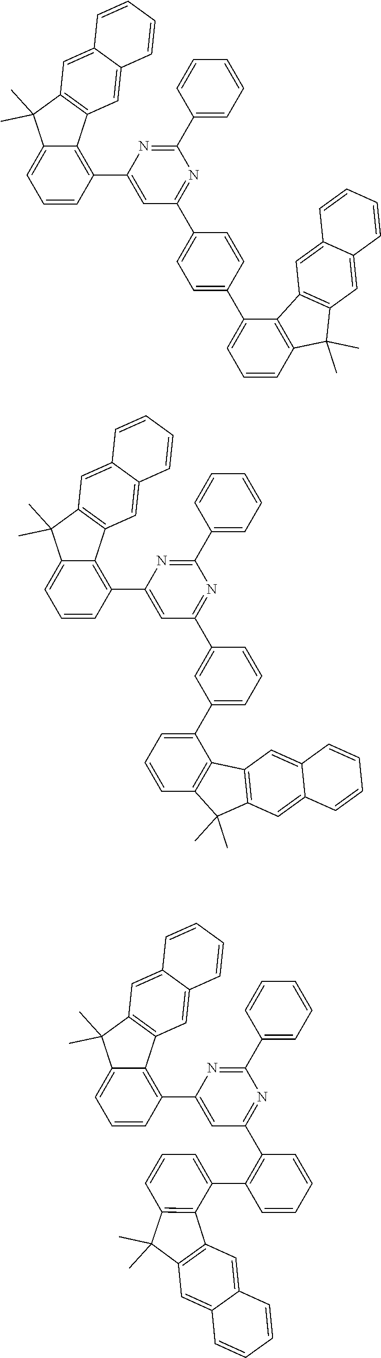 [SMILES: CC1(C)c2cc3ccccc3cc2-c2c(-c3ccc(-c4cc(-c5cccc6c5-c5cc7ccccc7cc5C6(C)C)nc(-c5ccccc5)n4)cc3)cccc21 ; CC1(C)c2cc3ccccc3cc2-c2c(-c3cccc(-c4cc(-c5cccc6c5-c5cc7ccccc7cc5C6(C)C)nc(-c5ccccc5)n4)c3)cccc21 ; CC1(C)c2cc3ccccc3cc2-c2c(-c3cc(-c4ccccc4-c4cccc5c4-c4cc6ccccc6cc4C5(C)C)nc(-c4ccccc4)n3)cccc21]

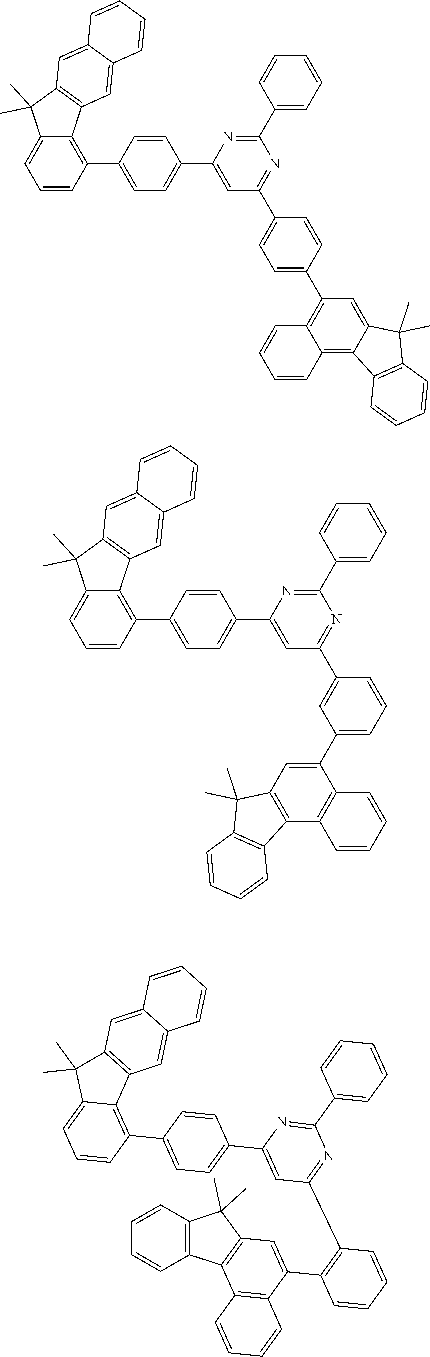

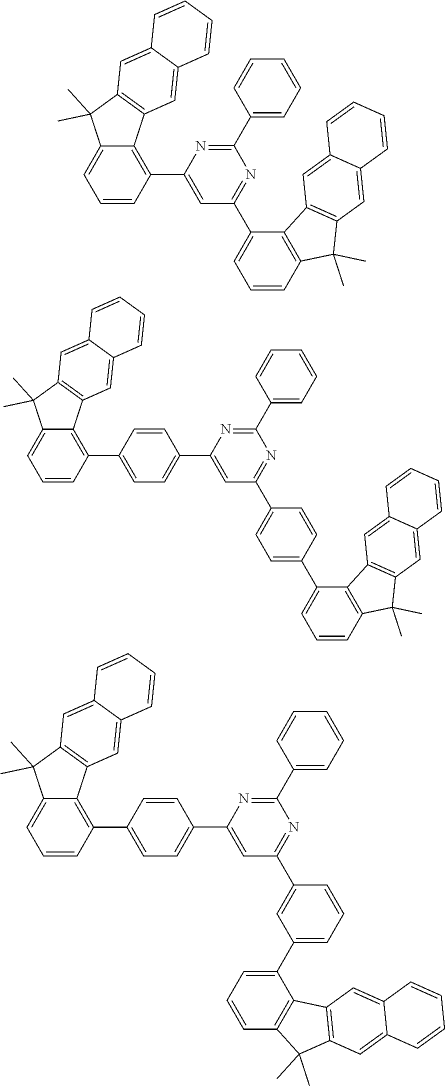 [SMILES: CC1(C)c2cc3ccccc3cc2-c2c(-c3cc(-c4cccc5c4-c4cc6ccccc6cc4C5(C)C)nc(-c4ccccc4)n3)cccc21 ; CC1(C)c2cc3ccccc3cc2-c2c(-c3ccc(-c4cc(-c5ccc(-c6cccc7c6-c6cc8ccccc8cc6C7(C)C)cc5)nc(-c5ccccc5)n4)cc3)cccc21 ; CC1(C)c2cc3ccccc3cc2-c2c(-c3ccc(-c4cc(-c5cccc(-c6cccc7c6-c6cc8ccccc8cc6C7(C)C)c5)nc(-c5ccccc5)n4)cc3)cccc21]

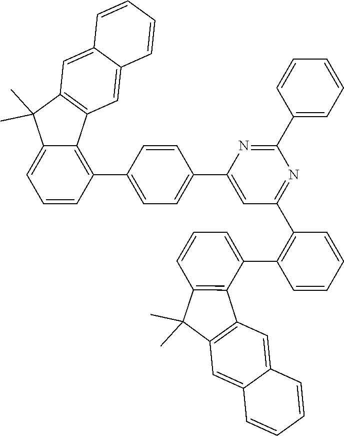 [SMILES: CC1(C)c2cc3ccccc3cc2-c2c(-c3ccc(-c4cc(-c5ccccc5-c5cccc6c5-c5cc7ccccc7cc5C6(C)C)nc(-c5ccccc5)n4)cc3)cccc21]

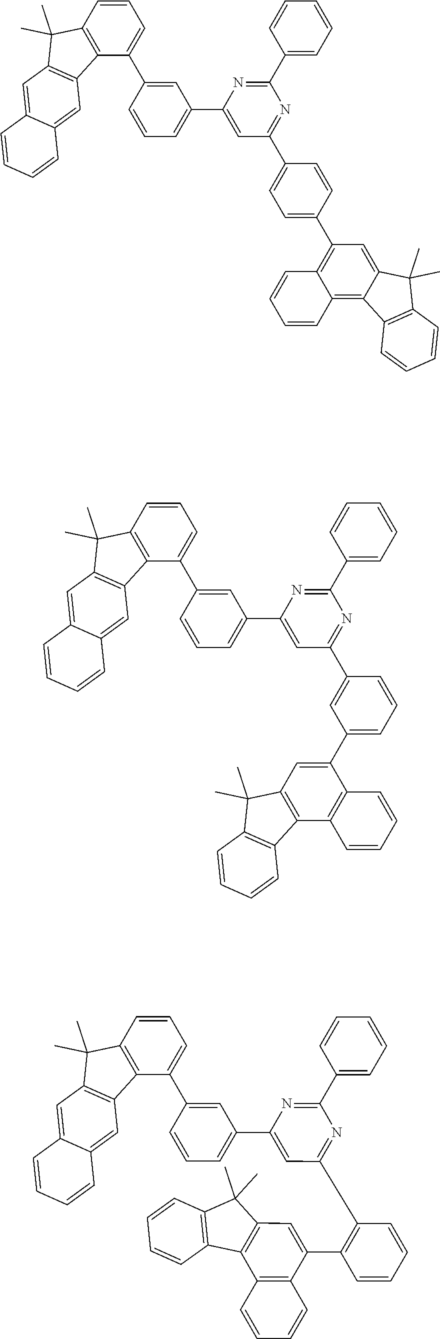 [SMILES: CC1(C)c2cc3ccccc3cc2-c2c(-c3cccc(-c4cc(-c5ccc(-c6cc7c(c8ccccc68)-c6ccccc6C7(C)C)cc5)nc(-c5ccccc5)n4)c3)cccc21 ; CC1(C)c2cc3ccccc3cc2-c2c(-c3cccc(-c4cc(-c5cccc(-c6cc7c(c8ccccc68)-c6ccccc6C7(C)C)c5)nc(-c5ccccc5)n4)c3)cccc21 ; CC1(C)c2cc3ccccc3cc2-c2c(-c3cccc(-c4cc(-c5ccccc5-c5cc6c(c7ccccc57)-c5ccccc5C6(C)C)nc(-c5ccccc5)n4)c3)cccc21]

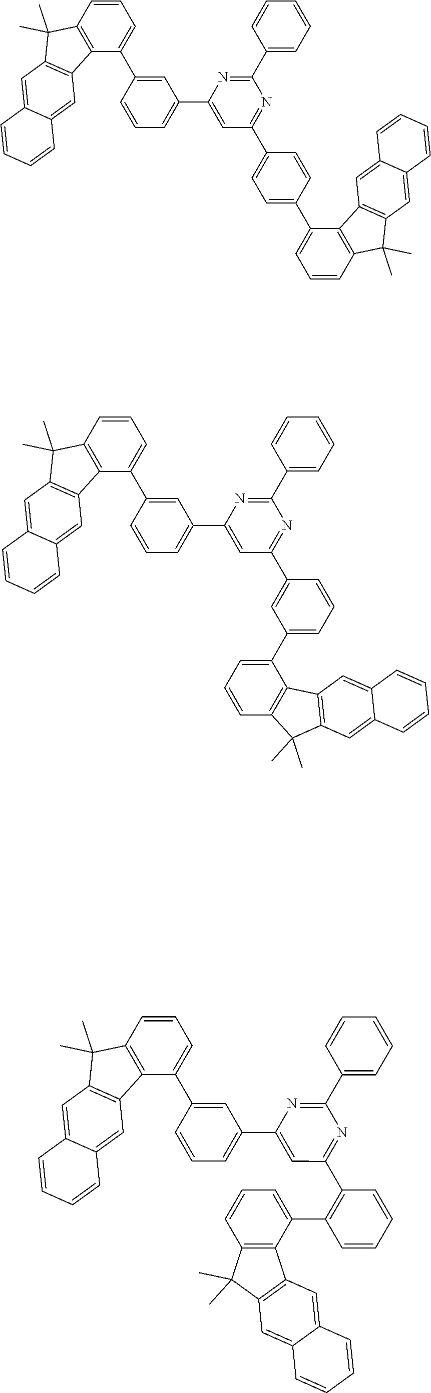 [SMILES: CC1(C)c2cc3ccccc3cc2-c2c(-c3ccc(-c4cc(-c5cccc(-c6cccc7c6-c6cc8ccccc8cc6C7(C)C)c5)nc(-c5ccccc5)n4)cc3)cccc21 ; CC1(C)c2cc3ccccc3cc2-c2c(-c3cccc(-c4cc(-c5cccc(-c6cccc7c6-c6cc8ccccc8cc6C7(C)C)c5)nc(-c5ccccc5)n4)c3)cccc21 ; CC1(C)c2cc3ccccc3cc2-c2c(-c3cccc(-c4cc(-c5ccccc5-c5cccc6c5-c5cc7ccccc7cc5C6(C)C)nc(-c5ccccc5)n4)c3)cccc21]

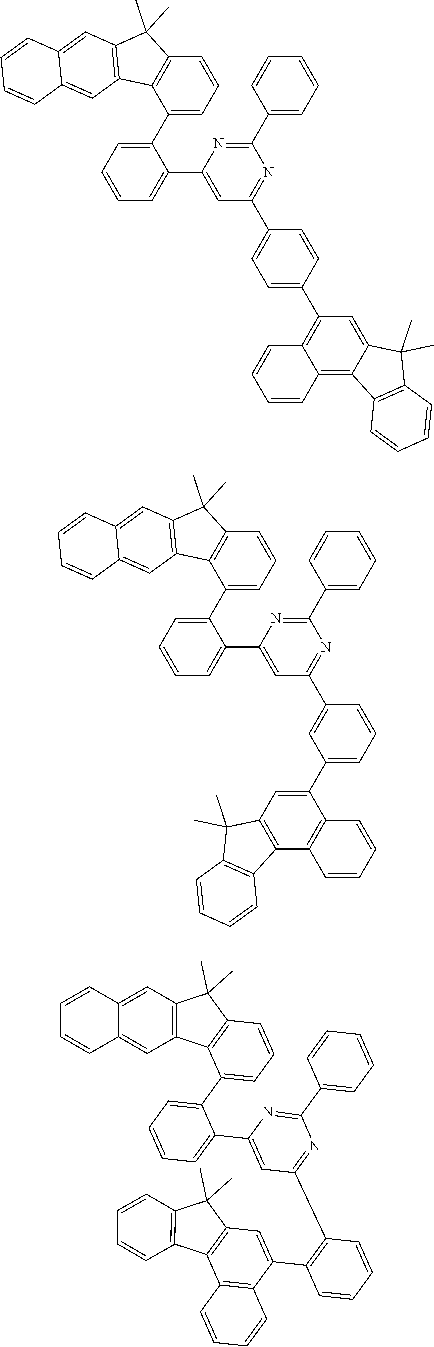

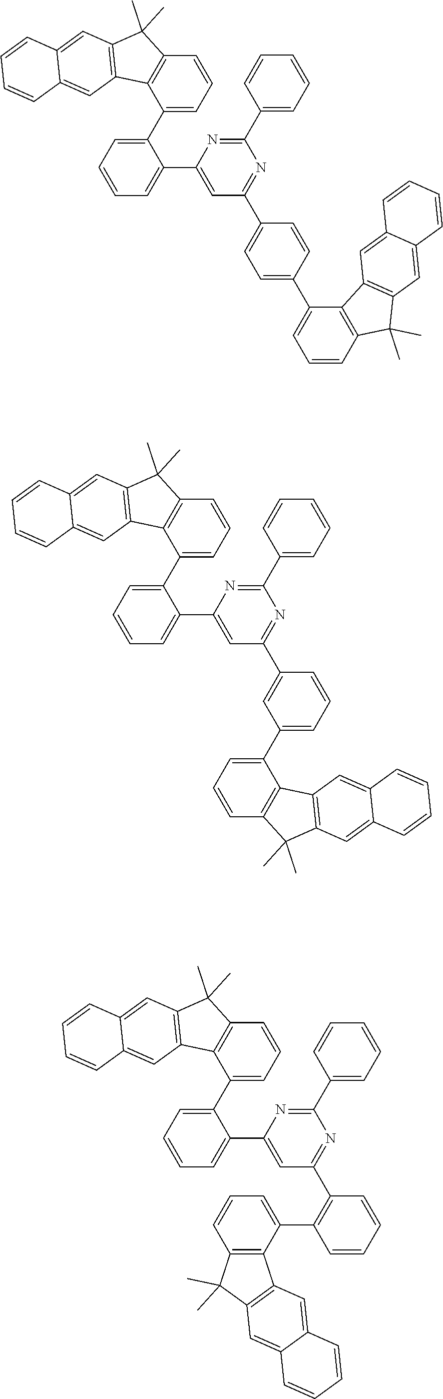 [SMILES: CC1(C)c2cc3ccccc3cc2-c2c(-c3ccc(-c4cc(-c5ccccc5-c5cccc6c5-c5cc7ccccc7cc5C6(C)C)nc(-c5ccccc5)n4)cc3)cccc21 ; CC1(C)c2cc3ccccc3cc2-c2c(-c3cccc(-c4cc(-c5ccccc5-c5cccc6c5-c5cc7ccccc7cc5C6(C)C)nc(-c5ccccc5)n4)c3)cccc21 ; CC1(C)c2cc3ccccc3cc2-c2c(-c3ccccc3-c3cc(-c4ccccc4-c4cccc5c4-c4cc6ccccc6cc4C5(C)C)nc(-c4ccccc4)n3)cccc21]

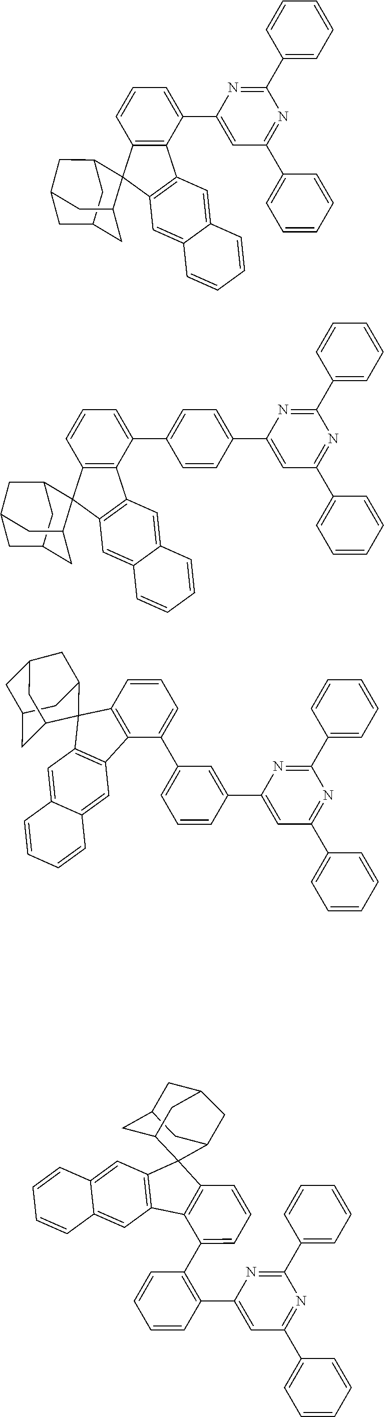 [SMILES: c1ccc(-c2cc(-c3cccc4c3-c3cc5ccccc5cc3C43C4CC5CC(C4)CC3C5)nc(-c3ccccc3)n2)cc1 ; c1ccc(-c2cc(-c3ccc(-c4cccc5c4-c4cc6ccccc6cc4C54C5CC6CC(C5)CC4C6)cc3)nc(-c3ccccc3)n2)cc1 ; c1ccc(-c2cc(-c3cccc(-c4cccc5c4-c4cc6ccccc6cc4C54C5CC6CC(C5)CC4C6)c3)nc(-c3ccccc3)n2)cc1 ; c1ccc(-c2cc(-c3ccccc3-c3cccc4c3-c3cc5ccccc5cc3C43C4CC5CC(C4)CC3C5)nc(-c3ccccc3)n2)cc1]

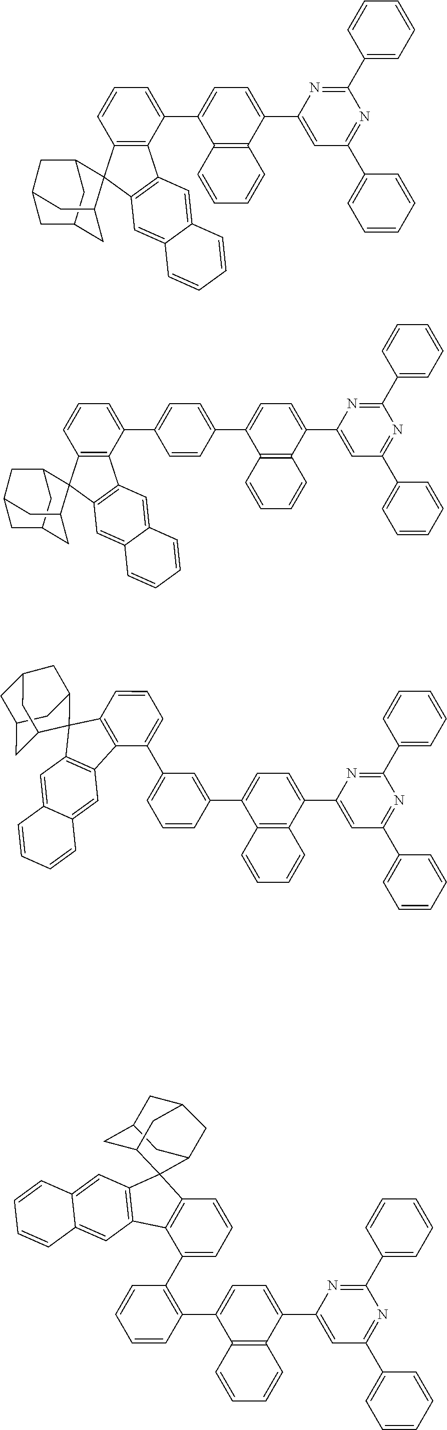 [SMILES: c1ccc(-c2cc(-c3ccc(-c4cccc5c4-c4cc6ccccc6cc4C54C5CC6CC(C5)CC4C6)c4ccccc34)nc(-c3ccccc3)n2)cc1 ; c1ccc(-c2cc(-c3ccc(-c4ccc(-c5cccc6c5-c5cc7ccccc7cc5C65C6CC7CC(C6)CC5C7)cc4)c4ccccc34)nc(-c3ccccc3)n2)cc1 ; c1ccc(-c2cc(-c3ccc(-c4cccc(-c5cccc6c5-c5cc7ccccc7cc5C65C6CC7CC(C6)CC5C7)c4)c4ccccc34)nc(-c3ccccc3)n2)cc1 ; c1ccc(-c2cc(-c3ccc(-c4ccccc4-c4cccc5c4-c4cc6ccccc6cc4C54C5CC6CC(C5)CC4C6)c4ccccc34)nc(-c3ccccc3)n2)cc1]

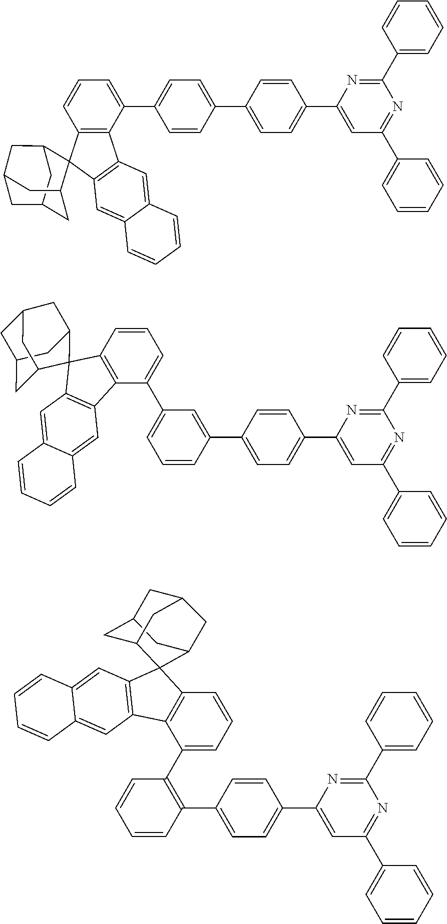 [SMILES: c1ccc(-c2cc(-c3ccc(-c4ccc(-c5cccc6c5-c5cc7ccccc7cc5C65C6CC7CC(C6)CC5C7)cc4)cc3)nc(-c3ccccc3)n2)cc1 ; c1ccc(-c2cc(-c3ccc(-c4cccc(-c5cccc6c5-c5cc7ccccc7cc5C65C6CC7CC(C6)CC5C7)c4)cc3)nc(-c3ccccc3)n2)cc1 ; c1ccc(-c2cc(-c3ccc(-c4ccccc4-c4cccc5c4-c4cc6ccccc6cc4C54C5CC6CC(C5)CC4C6)cc3)nc(-c3ccccc3)n2)cc1]

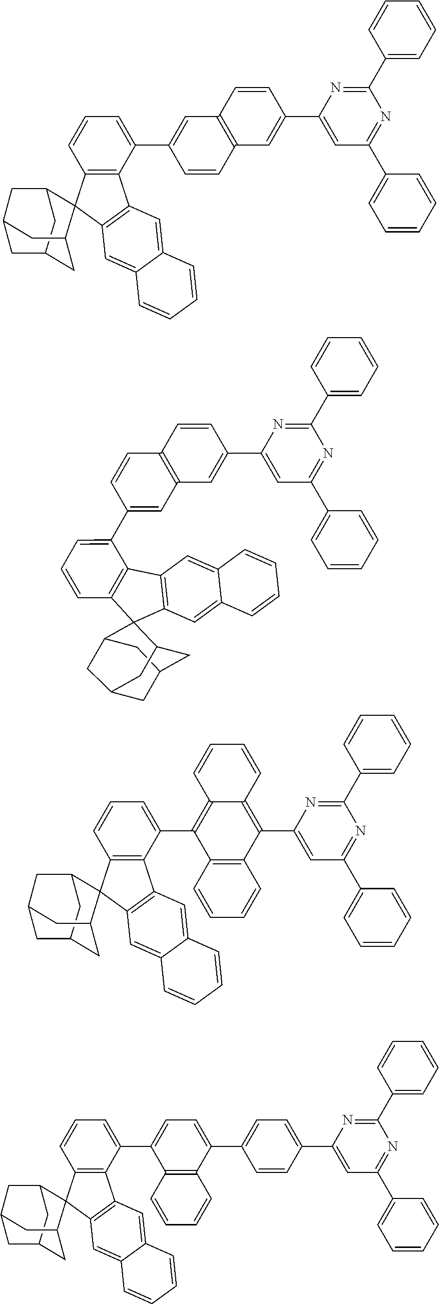 [SMILES: c1ccc(-c2cc(-c3ccc4cc(-c5cccc6c5-c5cc7ccccc7cc5C65C6CC7CC(C6)CC5C7)ccc4c3)nc(-c3ccccc3)n2)cc1 ; c1ccc(-c2cc(-c3ccc4ccc(-c5cccc6c5-c5cc7ccccc7cc5C65C6CC7CC(C6)CC5C7)cc4c3)nc(-c3ccccc3)n2)cc1 ; c1ccc(-c2cc(-c3c4ccccc4c(-c4cccc5c4-c4cc6ccccc6cc4C54C5CC6CC(C5)CC4C6)c4ccccc34)nc(-c3ccccc3)n2)cc1 ; c1ccc(-c2cc(-c3ccc(-c4ccc(-c5cccc6c5-c5cc7ccccc7cc5C65C6CC7CC(C6)CC5C7)c5ccccc45)cc3)nc(-c3ccccc3)n2)cc1]

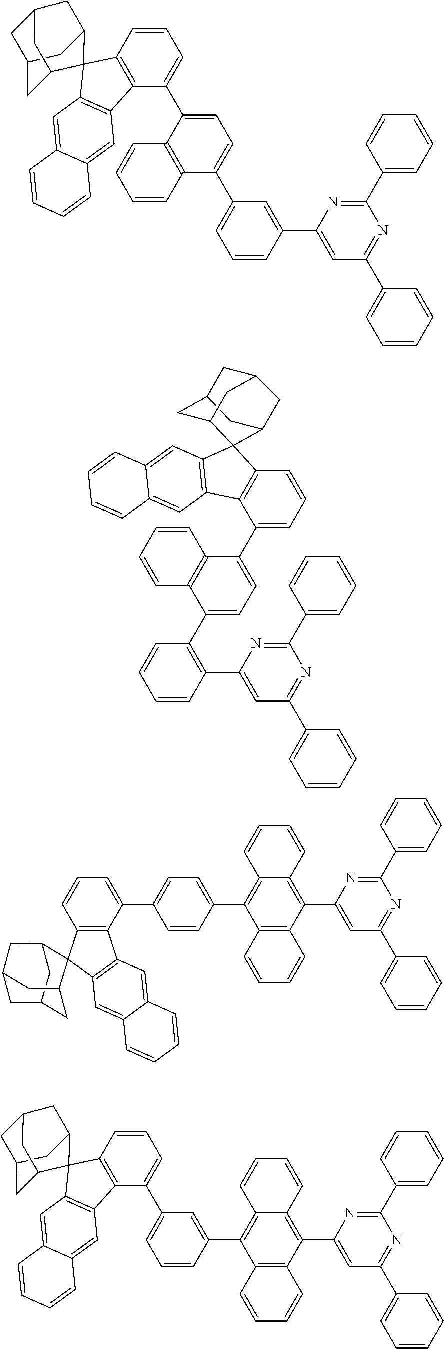

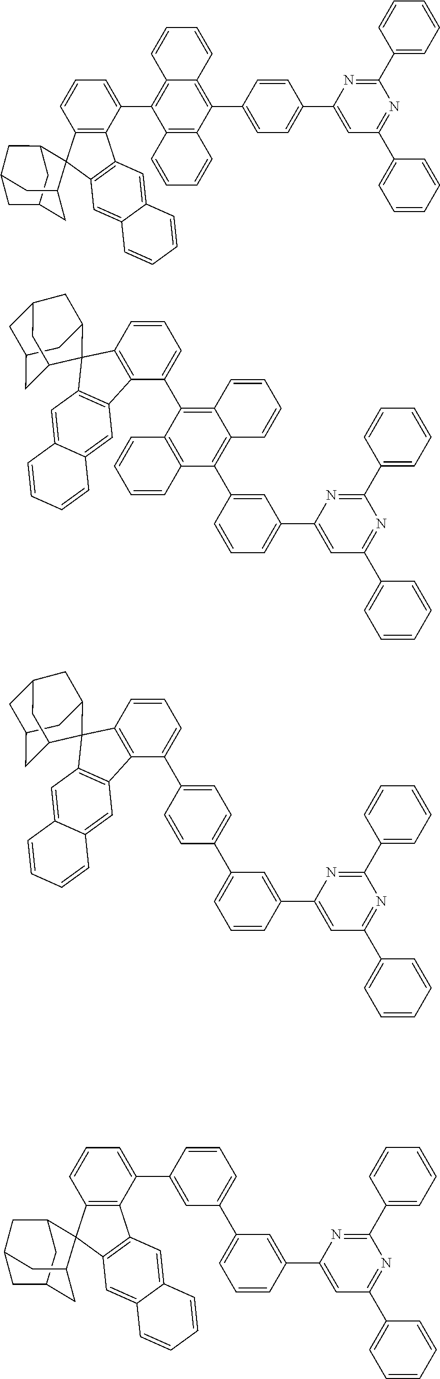 [SMILES: c1ccc(-c2cc(-c3ccc(-c4c5ccccc5c(-c5cccc6c5-c5cc7ccccc7cc5C65C6CC7CC(C6)CC5C7)c5ccccc45)cc3)nc(-c3ccccc3)n2)cc1 ; c1ccc(-c2cc(-c3cccc(-c4c5ccccc5c(-c5cccc6c5-c5cc7ccccc7cc5C65C6CC7CC(C6)CC5C7)c5ccccc45)c3)nc(-c3ccccc3)n2)cc1 ; c1ccc(-c2cc(-c3cccc(-c4ccc(-c5cccc6c5-c5cc7ccccc7cc5C65C6CC7CC(C6)CC5C7)cc4)c3)nc(-c3ccccc3)n2)cc1 ; c1ccc(-c2cc(-c3cccc(-c4cccc(-c5cccc6c5-c5cc7ccccc7cc5C65C6CC7CC(C6)CC5C7)c4)c3)nc(-c3ccccc3)n2)cc1]

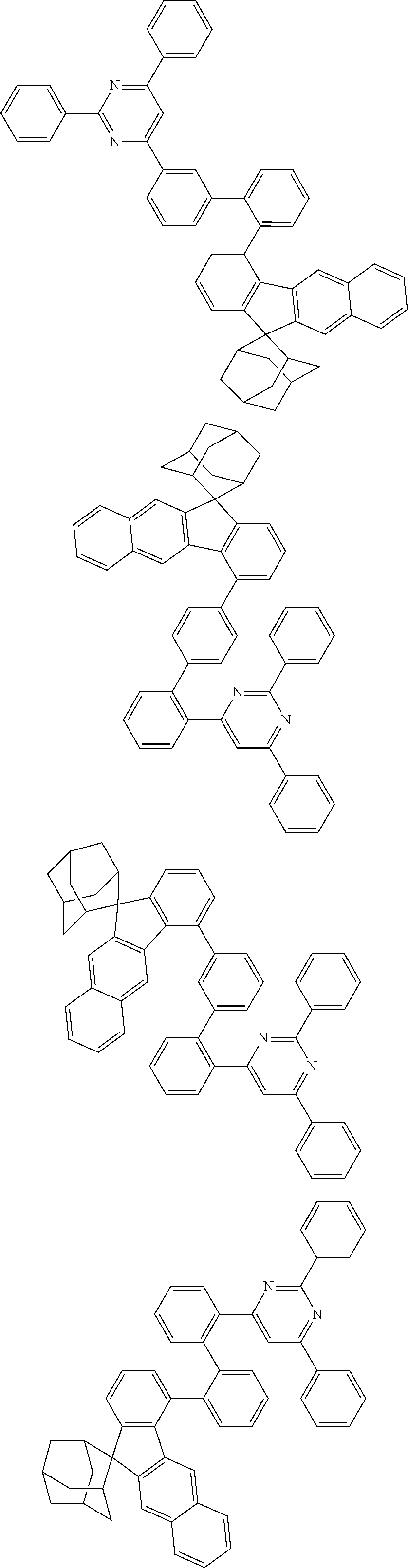

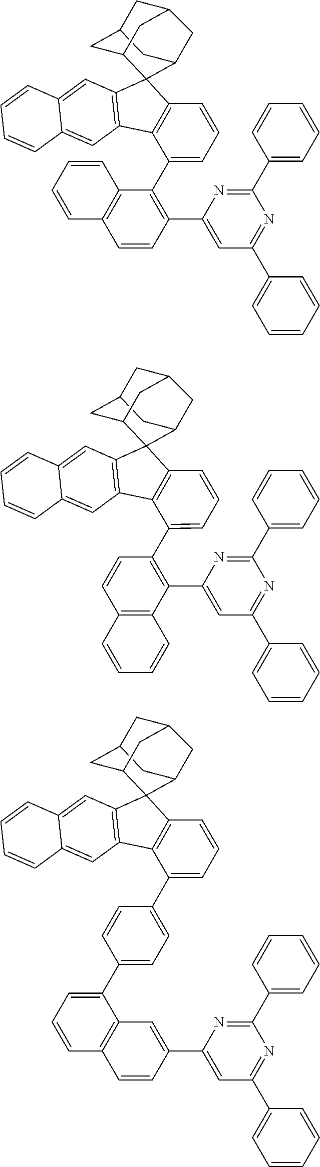 [SMILES: c1ccc(-c2cc(-c3ccc4ccccc4c3-c3cccc4c3-c3cc5ccccc5cc3C43C4CC5CC(C4)CC3C5)nc(-c3ccccc3)n2)cc1 ; c1ccc(-c2cc(-c3c(-c4cccc5c4-c4cc6ccccc6cc4C54C5CC6CC(C5)CC4C6)ccc4ccccc34)nc(-c3ccccc3)n2)cc1 ; c1ccc(-c2cc(-c3ccc4cccc(-c5ccc(-c6cccc7c6-c6cc8ccccc8cc6C76C7CC8CC(C7)CC6C8)cc5)c4c3)nc(-c3ccccc3)n2)cc1]

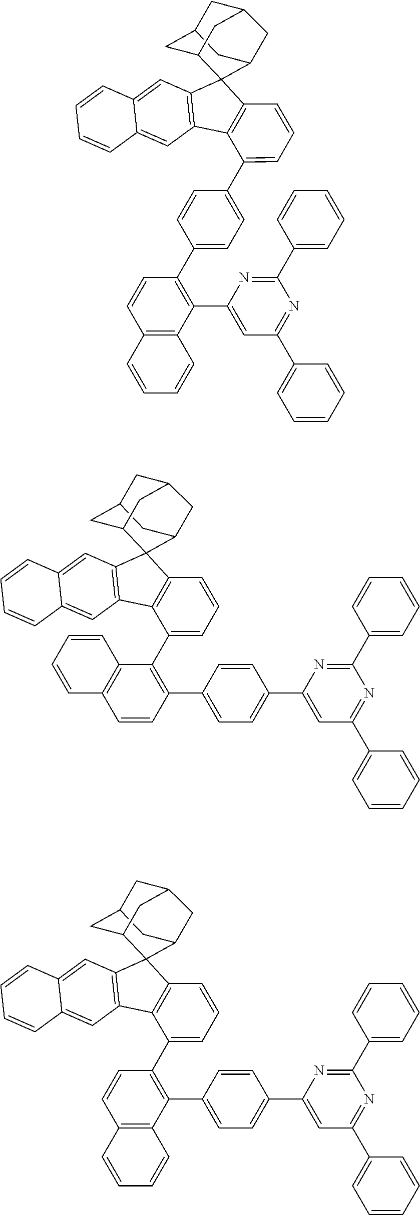 [SMILES: c1ccc(-c2cc(-c3c(-c4ccc(-c5cccc6c5-c5cc7ccccc7cc5C65C6CC7CC(C6)CC5C7)cc4)ccc4ccccc34)nc(-c3ccccc3)n2)cc1 ; c1ccc(-c2cc(-c3ccc(-c4ccc5ccccc5c4-c4cccc5c4-c4cc6ccccc6cc4C54C5CC6CC(C5)CC4C6)cc3)nc(-c3ccccc3)n2)cc1 ; c1ccc(-c2cc(-c3ccc(-c4c(-c5cccc6c5-c5cc7ccccc7cc5C65C6CC7CC(C6)CC5C7)ccc5ccccc45)cc3)nc(-c3ccccc3)n2)cc1]

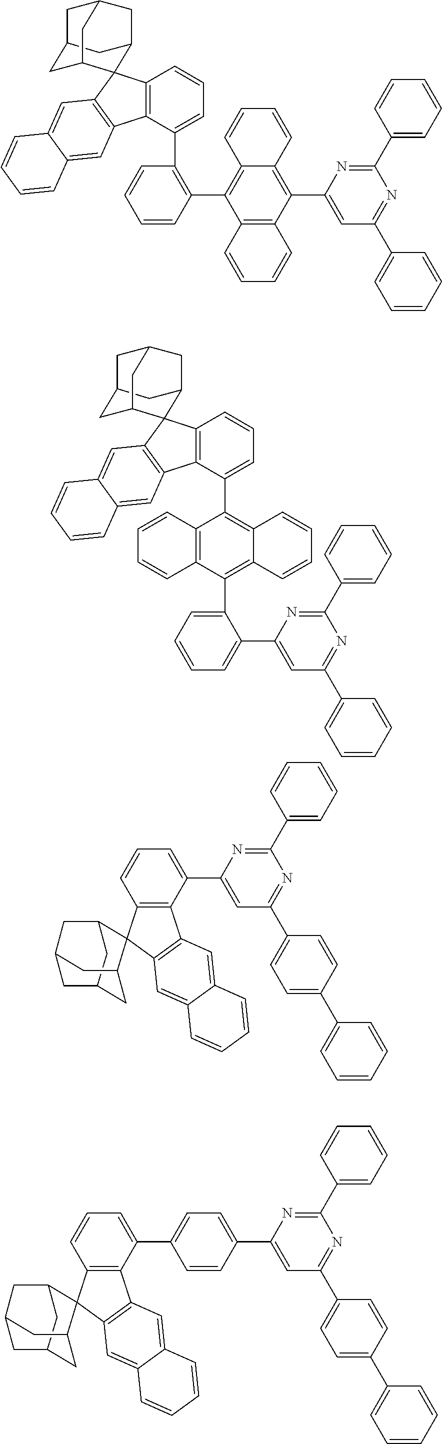

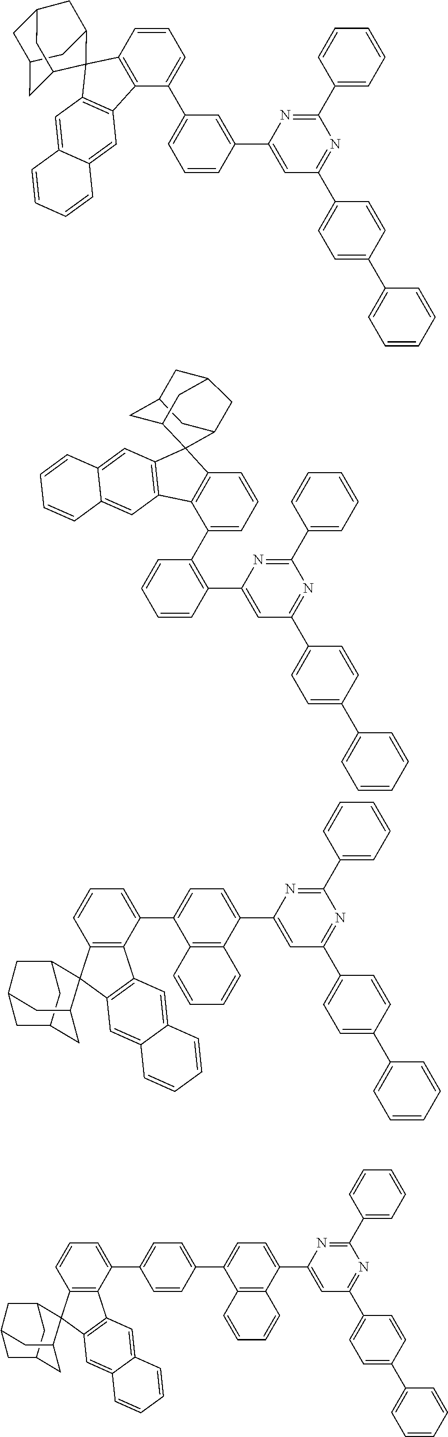

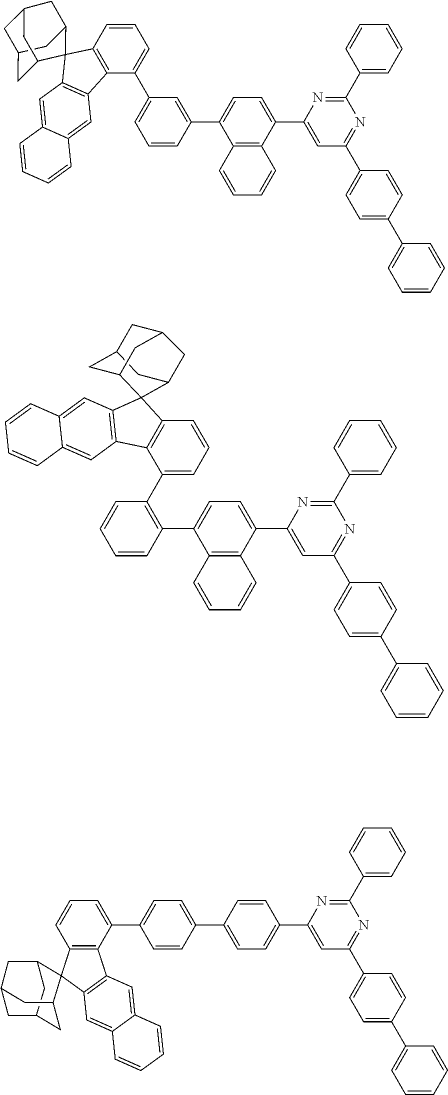 [SMILES: c1ccc(-c2ccc(-c3cc(-c4ccc(-c5cccc(-c6cccc7c6-c6cc8ccccc8cc6C76C7CC8CC(C7)CC6C8)c5)c5ccccc45)nc(-c4ccccc4)n3)cc2)cc1 ; c1ccc(-c2ccc(-c3cc(-c4ccc(-c5ccccc5-c5cccc6c5-c5cc7ccccc7cc5C65C6CC7CC(C6)CC5C7)c5ccccc45)nc(-c4ccccc4)n3)cc2)cc1 ; c1ccc(-c2ccc(-c3cc(-c4ccc(-c5ccc(-c6cccc7c6-c6cc8ccccc8cc6C76C7CC8CC(C7)CC6C8)cc5)cc4)nc(-c4ccccc4)n3)cc2)cc1]

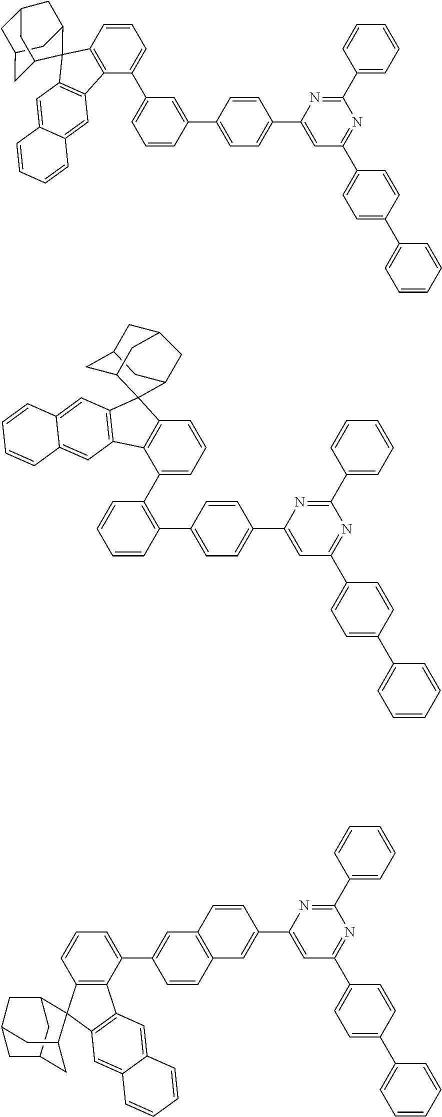 [SMILES: c1ccc(-c2ccc(-c3cc(-c4ccc(-c5cccc(-c6cccc7c6-c6cc8ccccc8cc6C76C7CC8CC(C7)CC6C8)c5)cc4)nc(-c4ccccc4)n3)cc2)cc1 ; c1ccc(-c2ccc(-c3cc(-c4ccc(-c5ccccc5-c5cccc6c5-c5cc7ccccc7cc5C65C6CC7CC(C6)CC5C7)cc4)nc(-c4ccccc4)n3)cc2)cc1 ; c1ccc(-c2ccc(-c3cc(-c4ccc5cc(-c6cccc7c6-c6cc8ccccc8cc6C76C7CC8CC(C7)CC6C8)ccc5c4)nc(-c4ccccc4)n3)cc2)cc1]

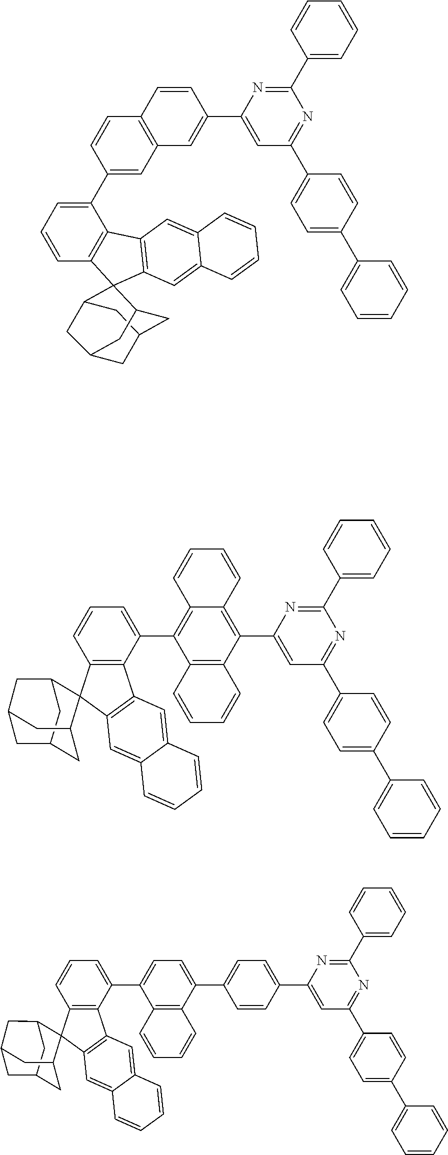 [SMILES: c1ccc(-c2ccc(-c3cc(-c4ccc5ccc(-c6cccc7c6-c6cc8ccccc8cc6C76C7CC8CC(C7)CC6C8)cc5c4)nc(-c4ccccc4)n3)cc2)cc1 ; c1ccc(-c2ccc(-c3cc(-c4c5ccccc5c(-c5cccc6c5-c5cc7ccccc7cc5C65C6CC7CC(C6)CC5C7)c5ccccc45)nc(-c4ccccc4)n3)cc2)cc1 ; c1ccc(-c2ccc(-c3cc(-c4ccc(-c5ccc(-c6cccc7c6-c6cc8ccccc8cc6C76C7CC8CC(C7)CC6C8)c6ccccc56)cc4)nc(-c4ccccc4)n3)cc2)cc1]

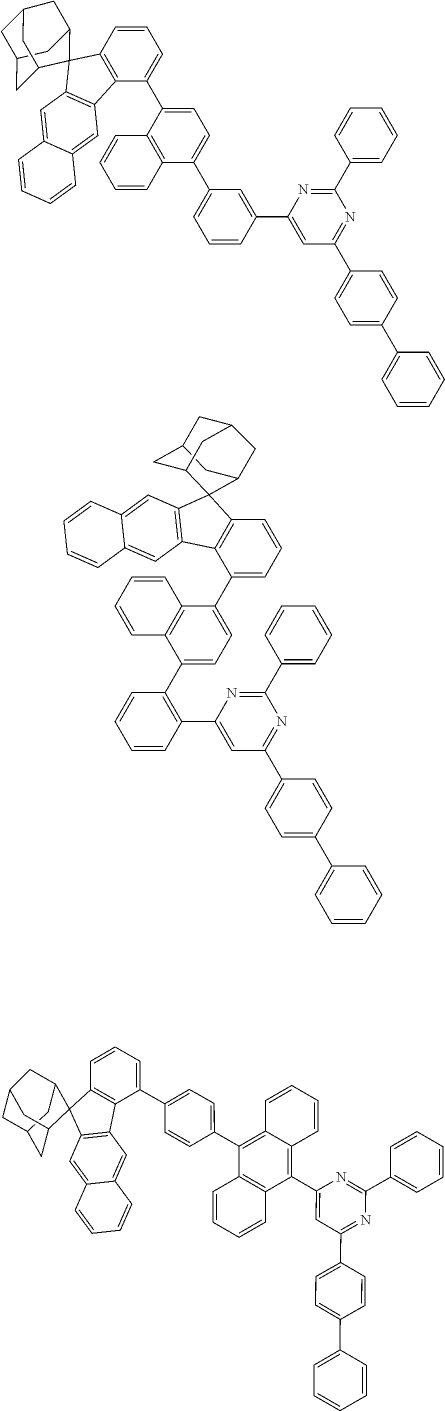 [SMILES: c1ccc(-c2ccc(-c3cc(-c4cccc(-c5ccc(-c6cccc7c6-c6cc8ccccc8cc6C76C7CC8CC(C7)CC6C8)c6ccccc56)c4)nc(-c4ccccc4)n3)cc2)cc1 ; c1ccc(-c2ccc(-c3cc(-c4ccccc4-c4ccc(-c5cccc6c5-c5cc7ccccc7cc5C65C6CC7CC(C6)CC5C7)c5ccccc45)nc(-c4ccccc4)n3)cc2)cc1 ; c1ccc(-c2ccc(-c3cc(-c4c5ccccc5c(-c5ccc(-c6cccc7c6-c6cc8ccccc8cc6C76C7CC8CC(C7)CC6C8)cc5)c5ccccc45)nc(-c4ccccc4)n3)cc2)cc1]

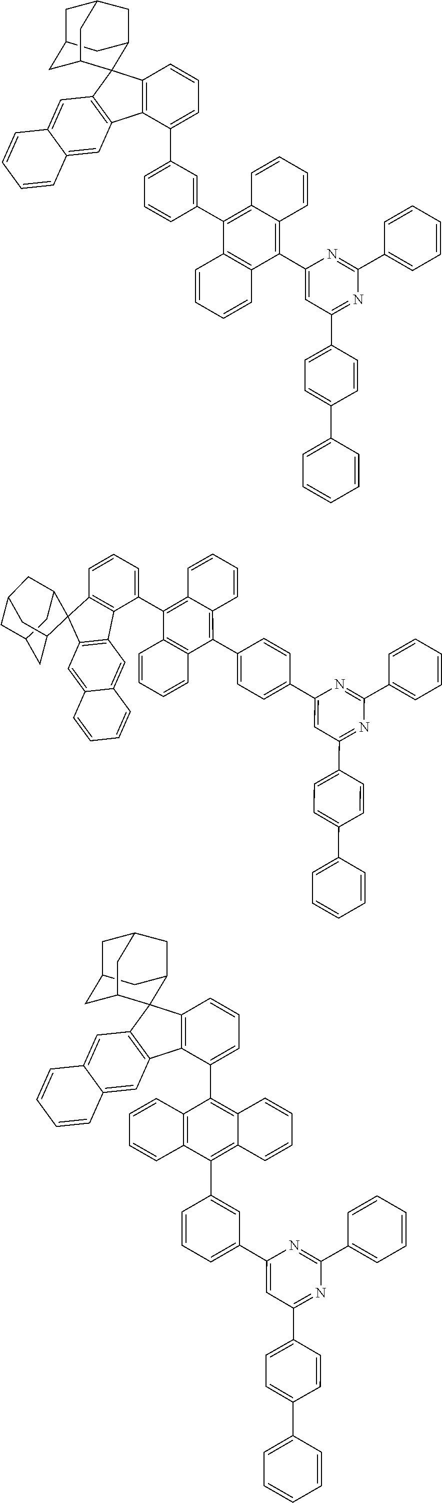 [SMILES: c1ccc(-c2ccc(-c3cc(-c4c5ccccc5c(-c5cccc(-c6cccc7c6-c6cc8ccccc8cc6C76C7CC8CC(C7)CC6C8)c5)c5ccccc45)nc(-c4ccccc4)n3)cc2)cc1 ; c1ccc(-c2ccc(-c3cc(-c4ccc(-c5c6ccccc6c(-c6cccc7c6-c6cc8ccccc8cc6C76C7CC8CC(C7)CC6C8)c6ccccc56)cc4)nc(-c4ccccc4)n3)cc2)cc1 ; c1ccc(-c2ccc(-c3cc(-c4cccc(-c5c6ccccc6c(-c6cccc7c6-c6cc8ccccc8cc6C76C7CC8CC(C7)CC6C8)c6ccccc56)c4)nc(-c4ccccc4)n3)cc2)cc1]

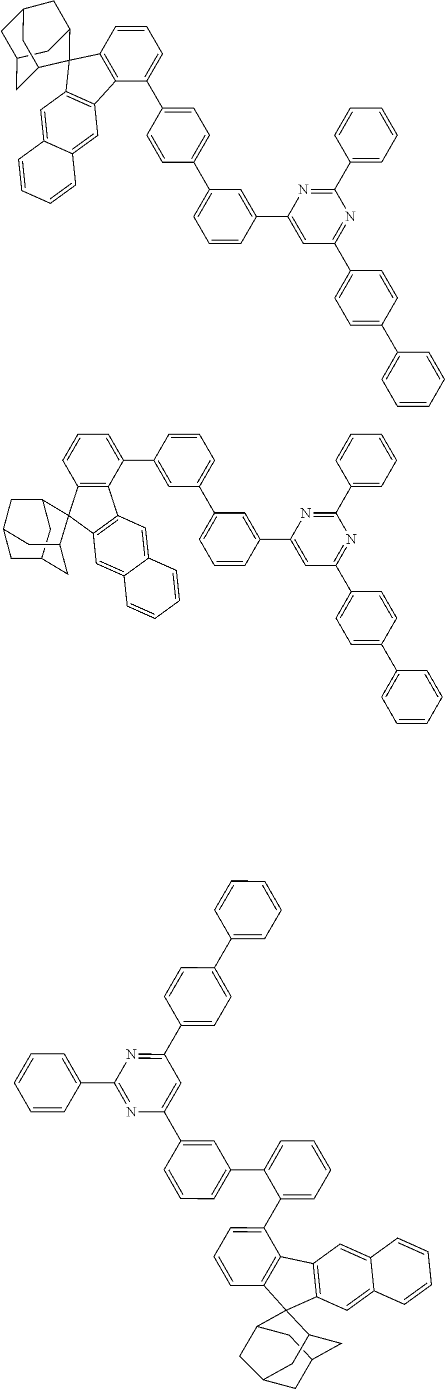

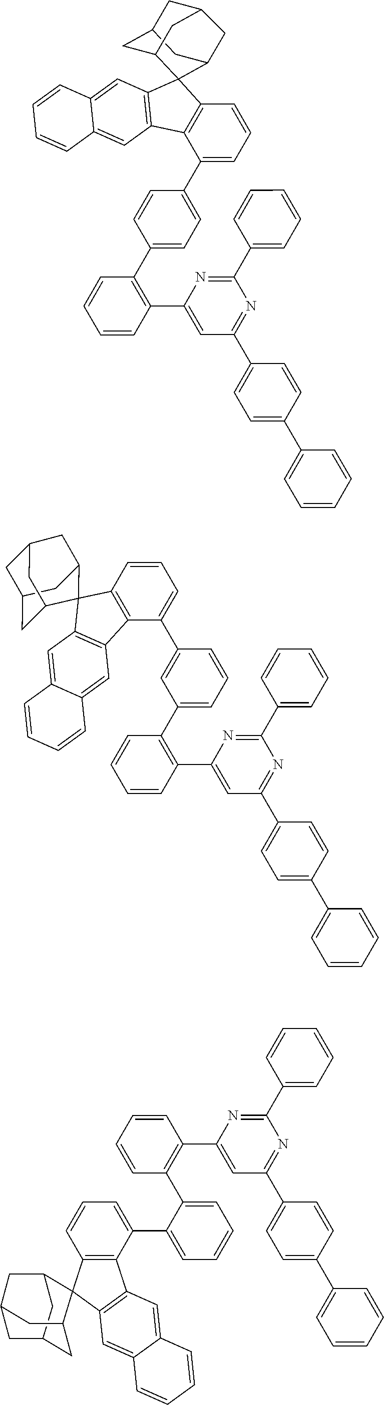 [SMILES: c1ccc(-c2ccc(-c3cc(-c4ccccc4-c4ccc(-c5cccc6c5-c5cc7ccccc7cc5C65C6CC7CC(C6)CC5C7)cc4)nc(-c4ccccc4)n3)cc2)cc1 ; c1ccc(-c2ccc(-c3cc(-c4ccccc4-c4cccc(-c5cccc6c5-c5cc7ccccc7cc5C65C6CC7CC(C6)CC5C7)c4)nc(-c4ccccc4)n3)cc2)cc1 ; c1ccc(-c2ccc(-c3cc(-c4ccccc4-c4ccccc4-c4cccc5c4-c4cc6ccccc6cc4C54C5CC6CC(C5)CC4C6)nc(-c4ccccc4)n3)cc2)cc1]

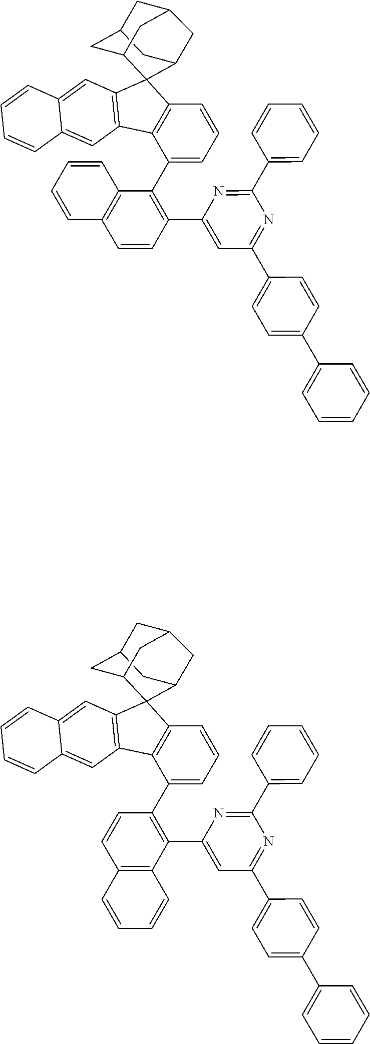 [SMILES: c1ccc(-c2ccc(-c3cc(-c4ccc5ccccc5c4-c4cccc5c4-c4cc6ccccc6cc4C54C5CC6CC(C5)CC4C6)nc(-c4ccccc4)n3)cc2)cc1 ; c1ccc(-c2ccc(-c3cc(-c4c(-c5cccc6c5-c5cc7ccccc7cc5C65C6CC7CC(C6)CC5C7)ccc5ccccc45)nc(-c4ccccc4)n3)cc2)cc1]

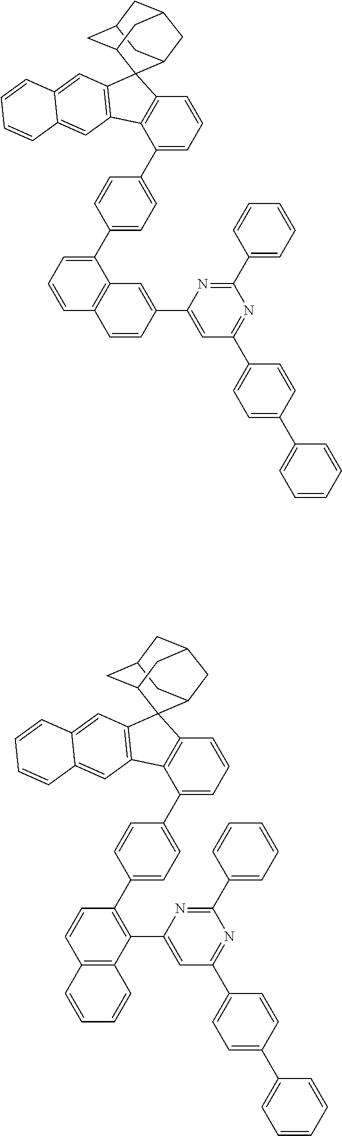 [SMILES: c1ccc(-c2ccc(-c3cc(-c4ccc5cccc(-c6ccc(-c7cccc8c7-c7cc9ccccc9cc7C87C8CC9CC(C8)CC7C9)cc6)c5c4)nc(-c4ccccc4)n3)cc2)cc1 ; c1ccc(-c2ccc(-c3cc(-c4c(-c5ccc(-c6cccc7c6-c6cc8ccccc8cc6C76C7CC8CC(C7)CC6C8)cc5)ccc5ccccc45)nc(-c4ccccc4)n3)cc2)cc1]

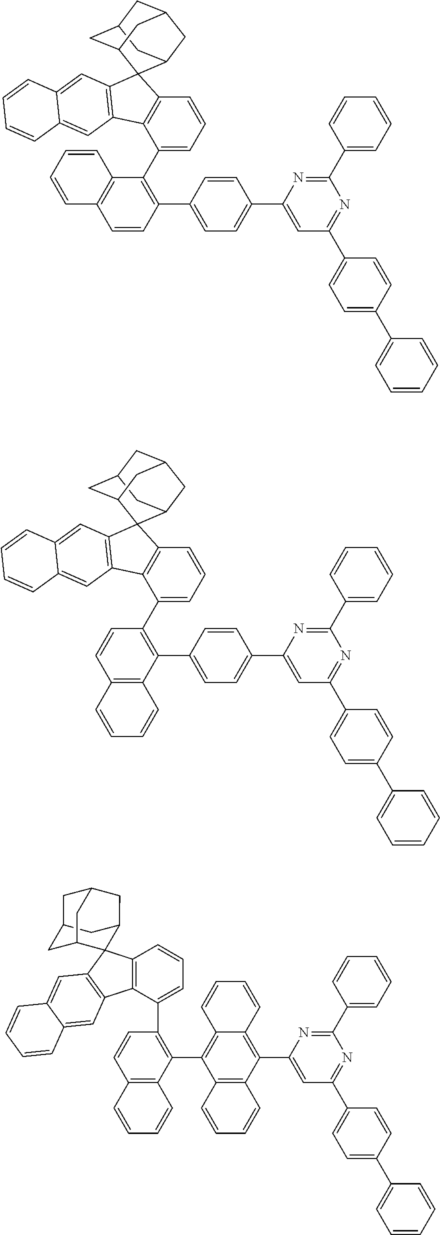 [SMILES: c1ccc(-c2ccc(-c3cc(-c4ccc(-c5ccc6ccccc6c5-c5cccc6c5-c5cc7ccccc7cc5C65C6CC7CC(C6)CC5C7)cc4)nc(-c4ccccc4)n3)cc2)cc1 ; c1ccc(-c2ccc(-c3cc(-c4ccc(-c5c(-c6cccc7c6-c6cc8ccccc8cc6C76C7CC8CC(C7)CC6C8)ccc6ccccc56)cc4)nc(-c4ccccc4)n3)cc2)cc1 ; c1ccc(-c2ccc(-c3cc(-c4c5ccccc5c(-c5c(-c6cccc7c6-c6cc8ccccc8cc6C76C7CC8CC(C7)CC6C8)ccc6ccccc56)c5ccccc45)nc(-c4ccccc4)n3)cc2)cc1]

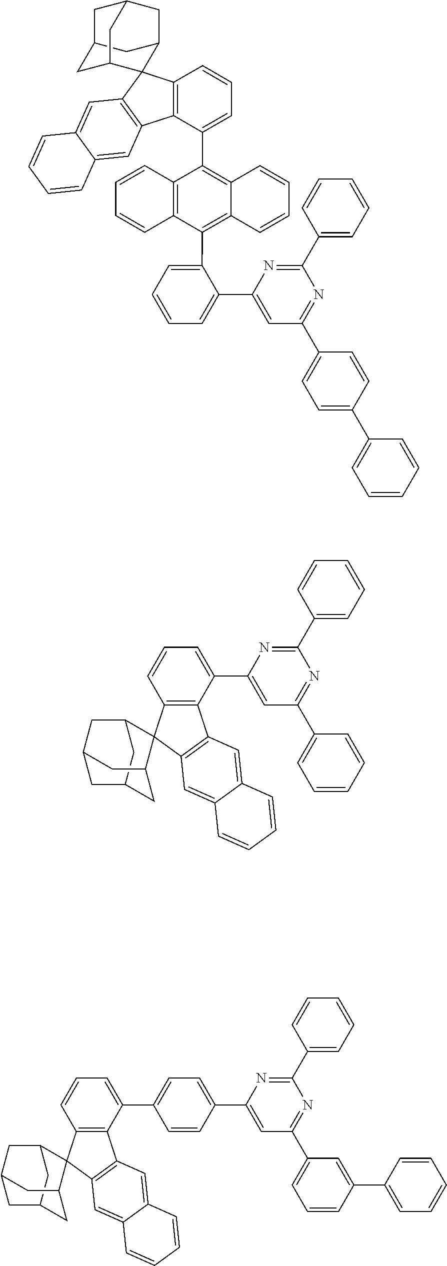 [SMILES: c1ccc(-c2ccc(-c3cc(-c4ccccc4-c4c5ccccc5c(-c5cccc6c5-c5cc7ccccc7cc5C65C6CC7CC(C6)CC5C7)c5ccccc45)nc(-c4ccccc4)n3)cc2)cc1 ; c1ccc(-c2cc(-c3cccc4c3-c3cc5ccccc5cc3C43C4CC5CC(C4)CC3C5)nc(-c3ccccc3)n2)cc1 ; c1ccc(-c2cccc(-c3cc(-c4ccc(-c5cccc6c5-c5cc7ccccc7cc5C65C6CC7CC(C6)CC5C7)cc4)nc(-c4ccccc4)n3)c2)cc1]

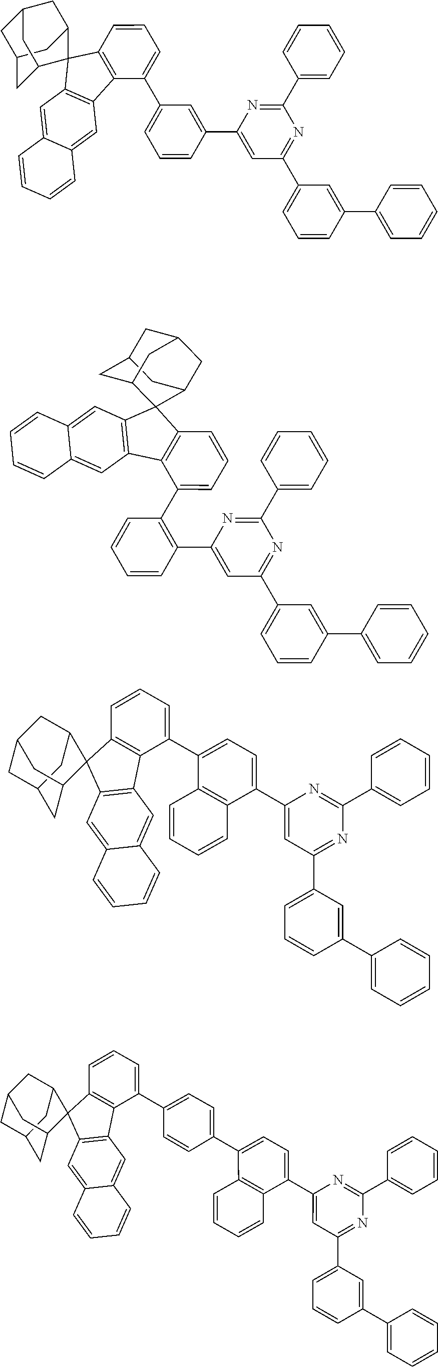 [SMILES: c1ccc(-c2cccc(-c3cc(-c4cccc(-c5cccc6c5-c5cc7ccccc7cc5C65C6CC7CC(C6)CC5C7)c4)nc(-c4ccccc4)n3)c2)cc1 ; c1ccc(-c2cccc(-c3cc(-c4ccccc4-c4cccc5c4-c4cc6ccccc6cc4C54C5CC6CC(C5)CC4C6)nc(-c4ccccc4)n3)c2)cc1 ; c1ccc(-c2cccc(-c3cc(-c4ccc(-c5cccc6c5-c5cc7ccccc7cc5C65C6CC7CC(C6)CC5C7)c5ccccc45)nc(-c4ccccc4)n3)c2)cc1 ; c1ccc(-c2cccc(-c3cc(-c4ccc(-c5ccc(-c6cccc7c6-c6cc8ccccc8cc6C76C7CC8CC(C7)CC6C8)cc5)c5ccccc45)nc(-c4ccccc4)n3)c2)cc1]

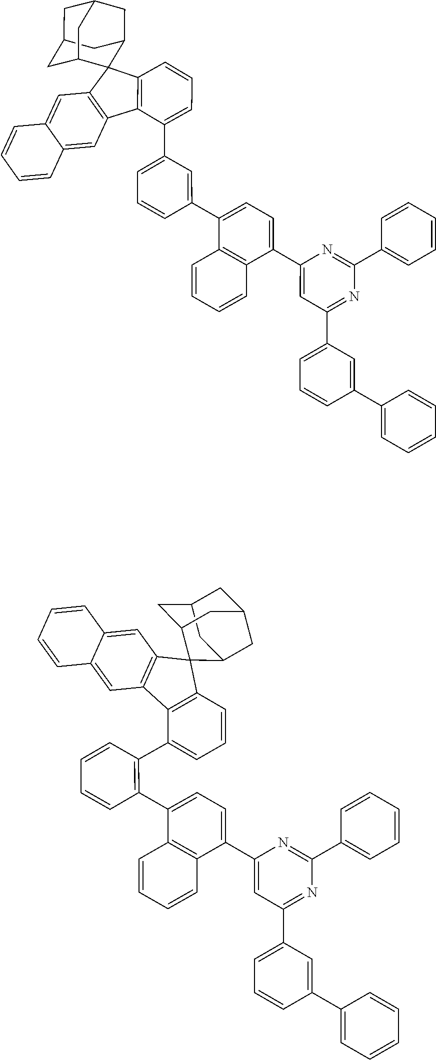 [SMILES: c1ccc(-c2cccc(-c3cc(-c4ccc(-c5cccc(-c6cccc7c6-c6cc8ccccc8cc6C76C7CC8CC(C7)CC6C8)c5)c5ccccc45)nc(-c4ccccc4)n3)c2)cc1 ; c1ccc(-c2cccc(-c3cc(-c4ccc(-c5ccccc5-c5cccc6c5-c5cc7ccccc7cc5C65C6CC7CC(C6)CC5C7)c5ccccc45)nc(-c4ccccc4)n3)c2)cc1]

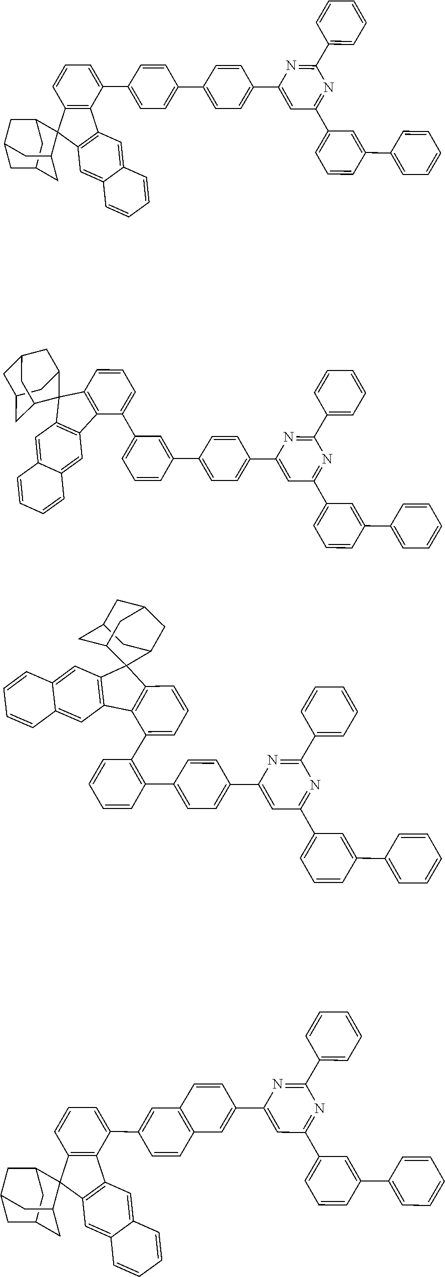 [SMILES: c1ccc(-c2cccc(-c3cc(-c4ccc(-c5ccc(-c6cccc7c6-c6cc8ccccc8cc6C76C7CC8CC(C7)CC6C8)cc5)cc4)nc(-c4ccccc4)n3)c2)cc1 ; c1ccc(-c2cccc(-c3cc(-c4ccc(-c5cccc(-c6cccc7c6-c6cc8ccccc8cc6C76C7CC8CC(C7)CC6C8)c5)cc4)nc(-c4ccccc4)n3)c2)cc1 ; c1ccc(-c2cccc(-c3cc(-c4ccc(-c5ccccc5-c5cccc6c5-c5cc7ccccc7cc5C65C6CC7CC(C6)CC5C7)cc4)nc(-c4ccccc4)n3)c2)cc1 ; c1ccc(-c2cccc(-c3cc(-c4ccc5cc(-c6cccc7c6-c6cc8ccccc8cc6C76C7CC8CC(C7)CC6C8)ccc5c4)nc(-c4ccccc4)n3)c2)cc1]

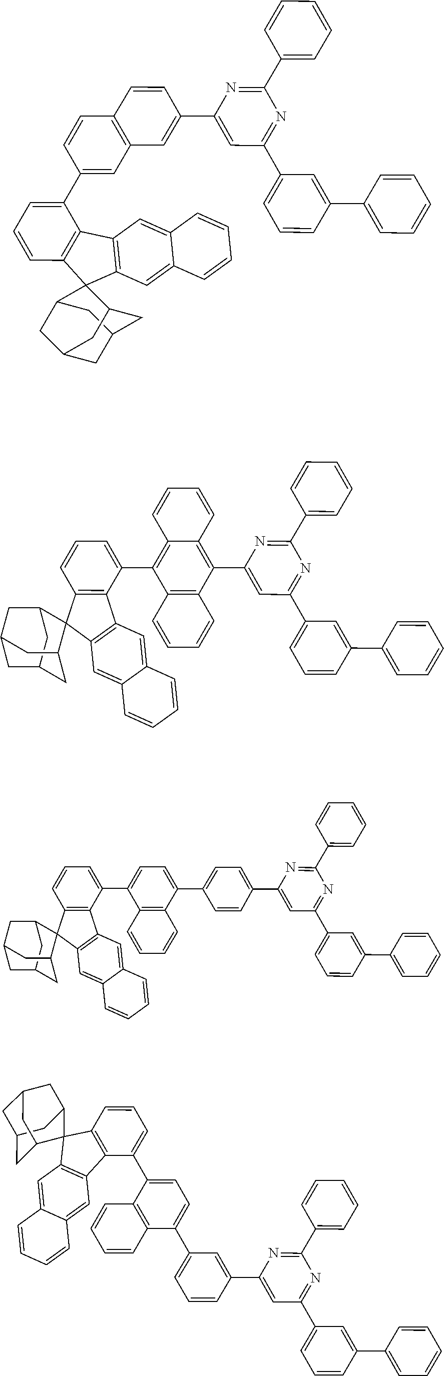 [SMILES: c1ccc(-c2cccc(-c3cc(-c4ccc5ccc(-c6cccc7c6-c6cc8ccccc8cc6C76C7CC8CC(C7)CC6C8)cc5c4)nc(-c4ccccc4)n3)c2)cc1 ; c1ccc(-c2cccc(-c3cc(-c4c5ccccc5c(-c5cccc6c5-c5cc7ccccc7cc5C65C6CC7CC(C6)CC5C7)c5ccccc45)nc(-c4ccccc4)n3)c2)cc1 ; c1ccc(-c2cccc(-c3cc(-c4ccc(-c5ccc(-c6cccc7c6-c6cc8ccccc8cc6C76C7CC8CC(C7)CC6C8)c6ccccc56)cc4)nc(-c4ccccc4)n3)c2)cc1 ; c1ccc(-c2cccc(-c3cc(-c4cccc(-c5ccc(-c6cccc7c6-c6cc8ccccc8cc6C76C7CC8CC(C7)CC6C8)c6ccccc56)c4)nc(-c4ccccc4)n3)c2)cc1]

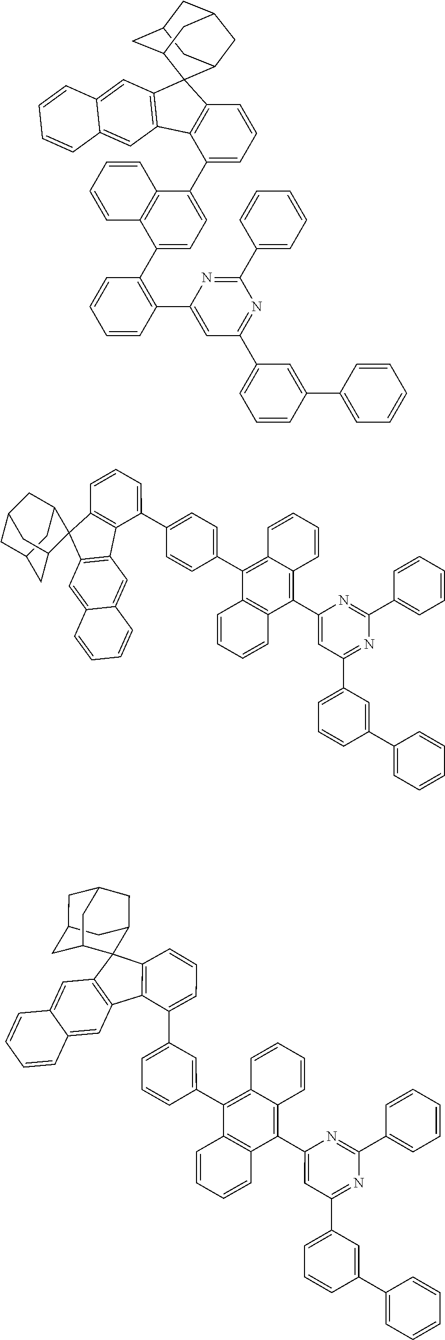 [SMILES: c1ccc(-c2cccc(-c3cc(-c4ccccc4-c4ccc(-c5cccc6c5-c5cc7ccccc7cc5C65C6CC7CC(C6)CC5C7)c5ccccc45)nc(-c4ccccc4)n3)c2)cc1 ; c1ccc(-c2cccc(-c3cc(-c4c5ccccc5c(-c5ccc(-c6cccc7c6-c6cc8ccccc8cc6C76C7CC8CC(C7)CC6C8)cc5)c5ccccc45)nc(-c4ccccc4)n3)c2)cc1 ; c1ccc(-c2cccc(-c3cc(-c4c5ccccc5c(-c5cccc(-c6cccc7c6-c6cc8ccccc8cc6C76C7CC8CC(C7)CC6C8)c5)c5ccccc45)nc(-c4ccccc4)n3)c2)cc1]

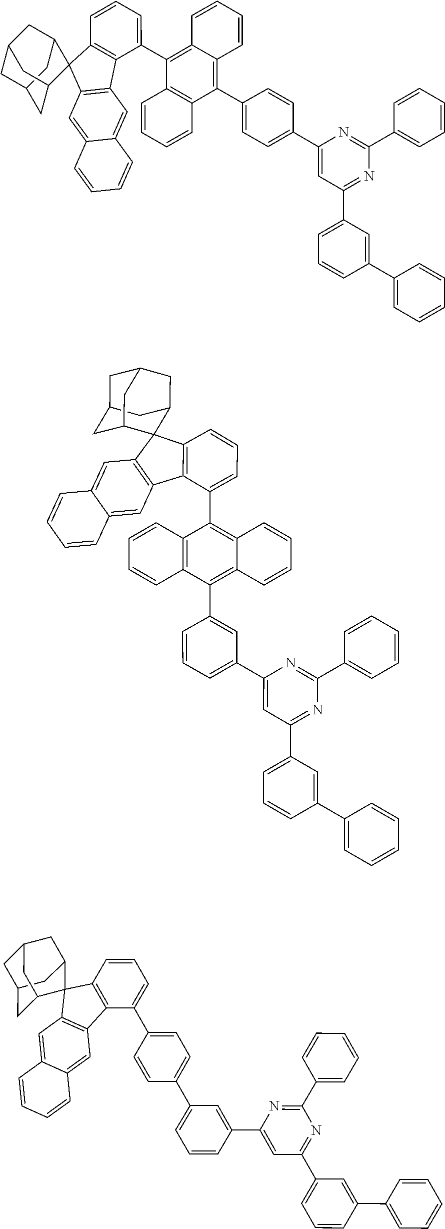 [SMILES: c1ccc(-c2cccc(-c3cc(-c4ccc(-c5c6ccccc6c(-c6cccc7c6-c6cc8ccccc8cc6C76C7CC8CC(C7)CC6C8)c6ccccc56)cc4)nc(-c4ccccc4)n3)c2)cc1 ; c1ccc(-c2cccc(-c3cc(-c4cccc(-c5c6ccccc6c(-c6cccc7c6-c6cc8ccccc8cc6C76C7CC8CC(C7)CC6C8)c6ccccc56)c4)nc(-c4ccccc4)n3)c2)cc1 ; c1ccc(-c2cccc(-c3cc(-c4cccc(-c5ccc(-c6cccc7c6-c6cc8ccccc8cc6C76C7CC8CC(C7)CC6C8)cc5)c4)nc(-c4ccccc4)n3)c2)cc1]

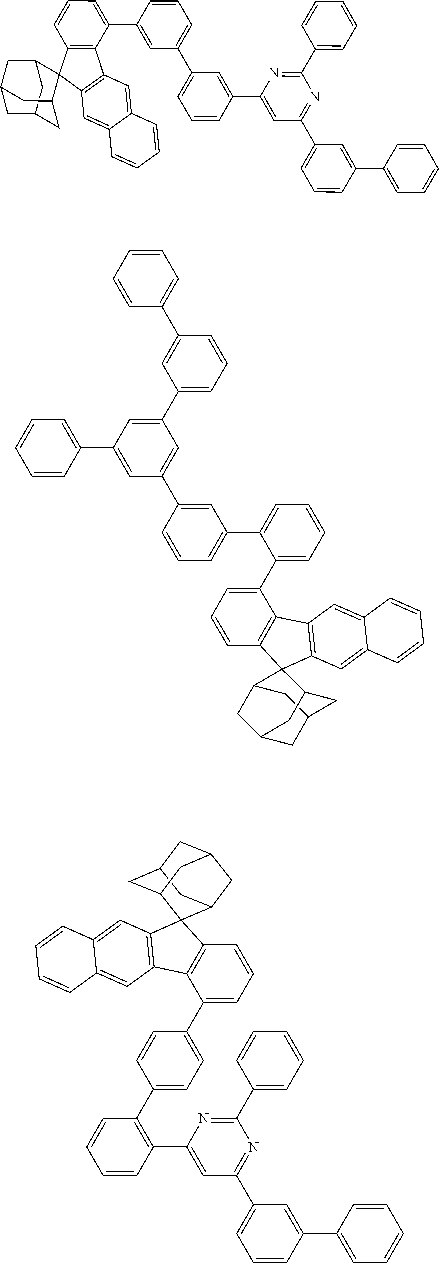

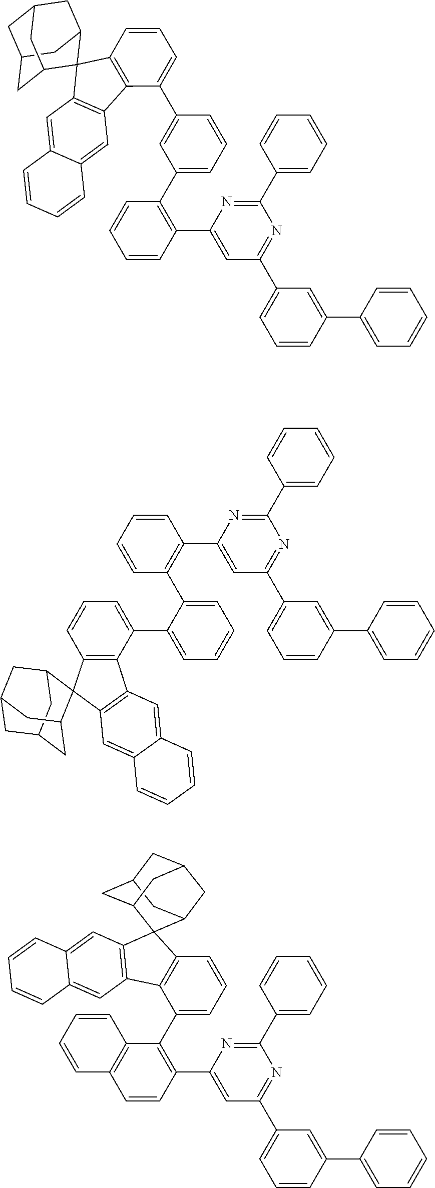 [SMILES: c1ccc(-c2cccc(-c3cc(-c4ccccc4-c4cccc(-c5cccc6c5-c5cc7ccccc7cc5C65C6CC7CC(C6)CC5C7)c4)nc(-c4ccccc4)n3)c2)cc1 ; c1ccc(-c2cccc(-c3cc(-c4ccccc4-c4ccccc4-c4cccc5c4-c4cc6ccccc6cc4C54C5CC6CC(C5)CC4C6)nc(-c4ccccc4)n3)c2)cc1 ; c1ccc(-c2cccc(-c3cc(-c4ccc5ccccc5c4-c4cccc5c4-c4cc6ccccc6cc4C54C5CC6CC(C5)CC4C6)nc(-c4ccccc4)n3)c2)cc1]

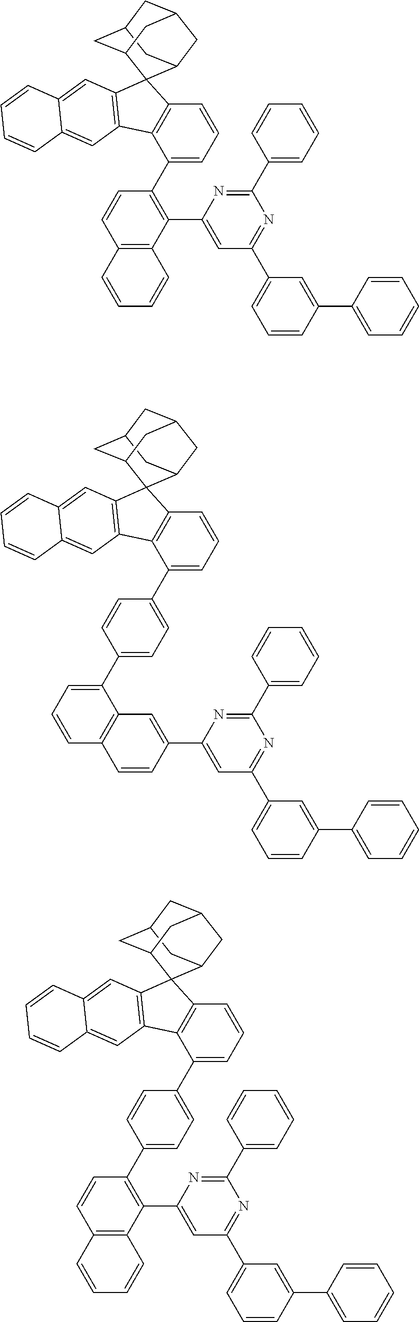 [SMILES: c1ccc(-c2cccc(-c3cc(-c4c(-c5cccc6c5-c5cc7ccccc7cc5C65C6CC7CC(C6)CC5C7)ccc5ccccc45)nc(-c4ccccc4)n3)c2)cc1 ; c1ccc(-c2cccc(-c3cc(-c4ccc5cccc(-c6ccc(-c7cccc8c7-c7cc9ccccc9cc7C87C8CC9CC(C8)CC7C9)cc6)c5c4)nc(-c4ccccc4)n3)c2)cc1 ; c1ccc(-c2cccc(-c3cc(-c4c(-c5ccc(-c6cccc7c6-c6cc8ccccc8cc6C76C7CC8CC(C7)CC6C8)cc5)ccc5ccccc45)nc(-c4ccccc4)n3)c2)cc1]

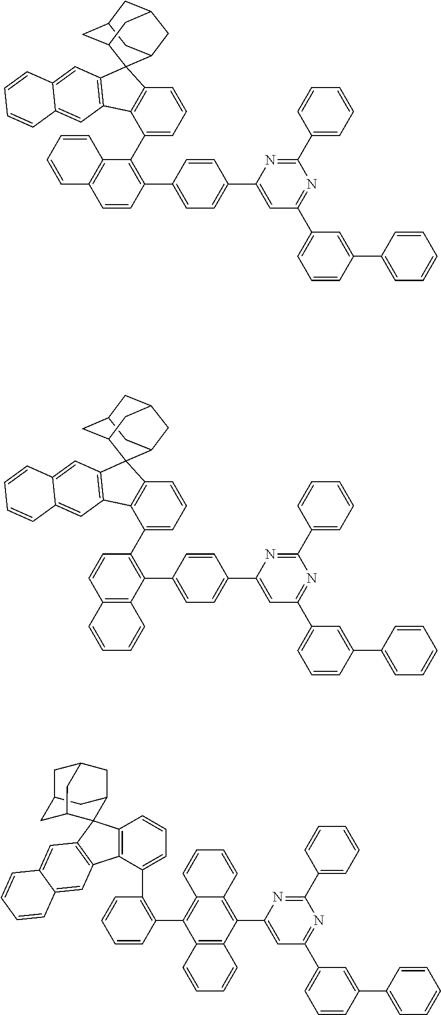 [SMILES: c1ccc(-c2cccc(-c3cc(-c4ccc(-c5ccc6ccccc6c5-c5cccc6c5-c5cc7ccccc7cc5C65C6CC7CC(C6)CC5C7)cc4)nc(-c4ccccc4)n3)c2)cc1 ; c1ccc(-c2cccc(-c3cc(-c4ccc(-c5c(-c6cccc7c6-c6cc8ccccc8cc6C76C7CC8CC(C7)CC6C8)ccc6ccccc56)cc4)nc(-c4ccccc4)n3)c2)cc1 ; c1ccc(-c2cccc(-c3cc(-c4c5ccccc5c(-c5ccccc5-c5cccc6c5-c5cc7ccccc7cc5C65C6CC7CC(C6)CC5C7)c5ccccc45)nc(-c4ccccc4)n3)c2)cc1]

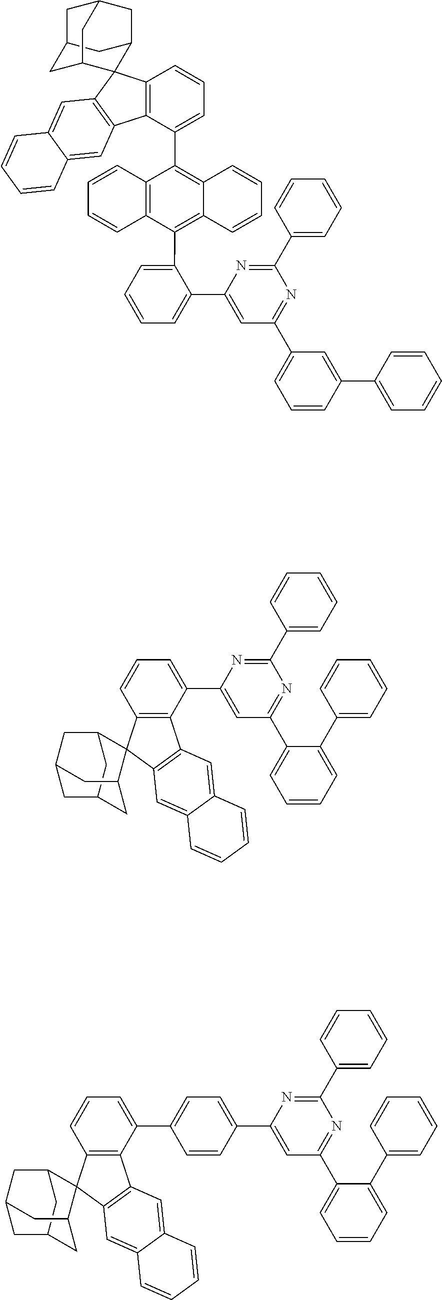 [SMILES: c1ccc(-c2cccc(-c3cc(-c4ccccc4-c4c5ccccc5c(-c5cccc6c5-c5cc7ccccc7cc5C65C6CC7CC(C6)CC5C7)c5ccccc45)nc(-c4ccccc4)n3)c2)cc1 ; c1ccc(-c2nc(-c3ccccc3-c3ccccc3)cc(-c3cccc4c3-c3cc5ccccc5cc3C43C4CC5CC(C4)CC3C5)n2)cc1 ; c1ccc(-c2nc(-c3ccc(-c4cccc5c4-c4cc6ccccc6cc4C54C5CC6CC(C5)CC4C6)cc3)cc(-c3ccccc3-c3ccccc3)n2)cc1]

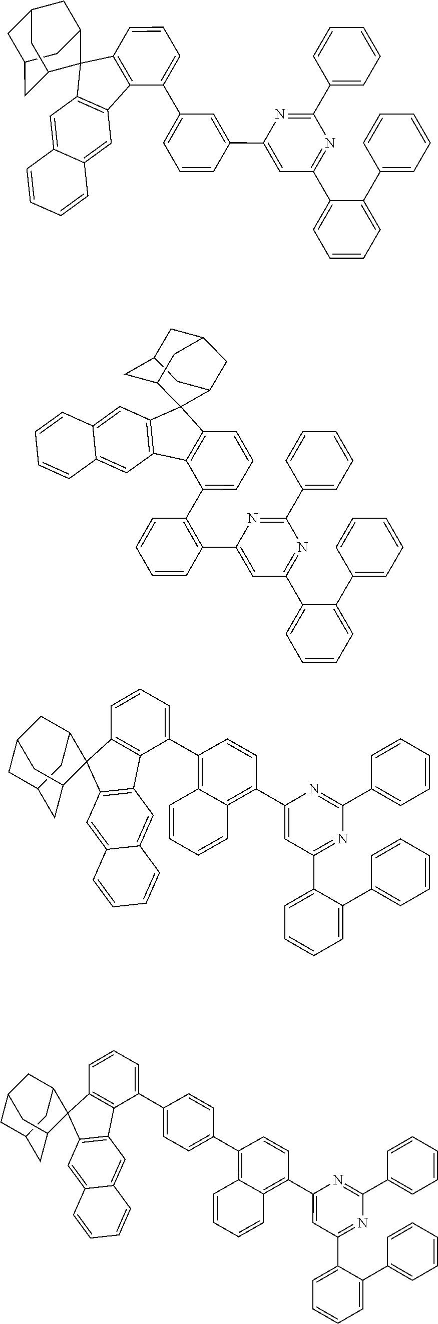 [SMILES: c1ccc(-c2nc(-c3cccc(-c4cccc5c4-c4cc6ccccc6cc4C54C5CC6CC(C5)CC4C6)c3)cc(-c3ccccc3-c3ccccc3)n2)cc1 ; c1ccc(-c2nc(-c3ccccc3-c3ccccc3)cc(-c3ccccc3-c3cccc4c3-c3cc5ccccc5cc3C43C4CC5CC(C4)CC3C5)n2)cc1 ; c1ccc(-c2nc(-c3ccccc3-c3ccccc3)cc(-c3ccc(-c4cccc5c4-c4cc6ccccc6cc4C54C5CC6CC(C5)CC4C6)c4ccccc34)n2)cc1 ; c1ccc(-c2nc(-c3ccccc3-c3ccccc3)cc(-c3ccc(-c4ccc(-c5cccc6c5-c5cc7ccccc7cc5C65C6CC7CC(C6)CC5C7)cc4)c4ccccc34)n2)cc1]

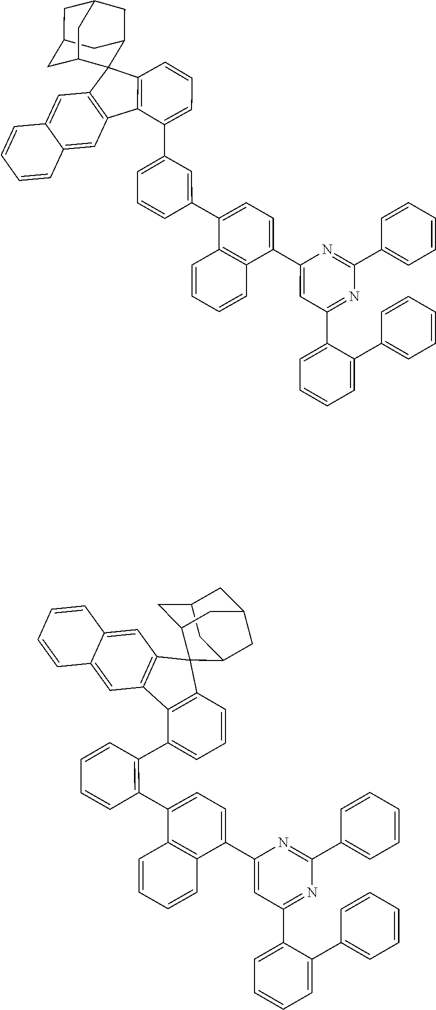 [SMILES: c1ccc(-c2nc(-c3ccccc3-c3ccccc3)cc(-c3ccc(-c4cccc(-c5cccc6c5-c5cc7ccccc7cc5C65C6CC7CC(C6)CC5C7)c4)c4ccccc34)n2)cc1 ; c1ccc(-c2nc(-c3ccccc3-c3ccccc3)cc(-c3ccc(-c4ccccc4-c4cccc5c4-c4cc6ccccc6cc4C54C5CC6CC(C5)CC4C6)c4ccccc34)n2)cc1]

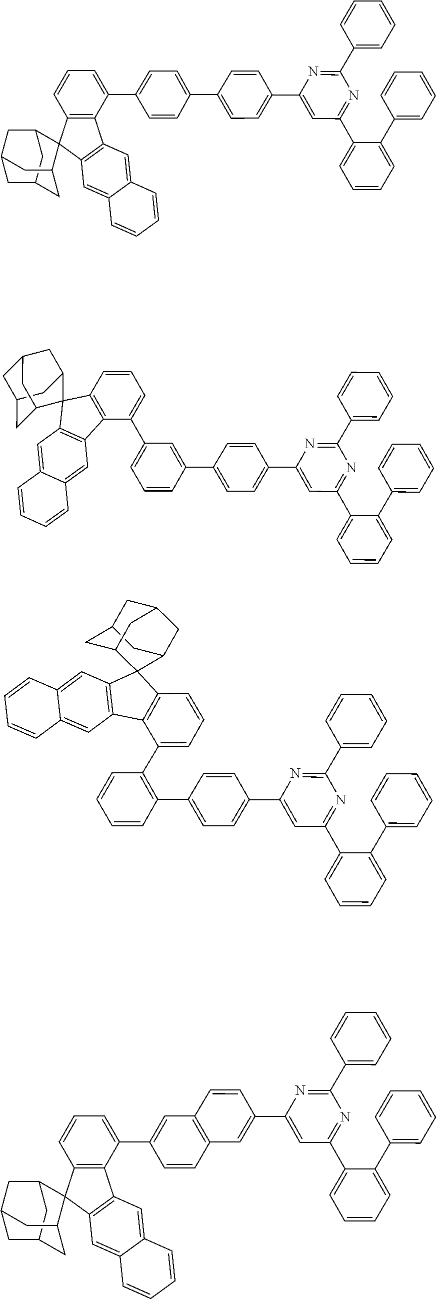 [SMILES: c1ccc(-c2nc(-c3ccc(-c4ccc(-c5cccc6c5-c5cc7ccccc7cc5C65C6CC7CC(C6)CC5C7)cc4)cc3)cc(-c3ccccc3-c3ccccc3)n2)cc1 ; c1ccc(-c2nc(-c3ccc(-c4cccc(-c5cccc6c5-c5cc7ccccc7cc5C65C6CC7CC(C6)CC5C7)c4)cc3)cc(-c3ccccc3-c3ccccc3)n2)cc1 ; c1ccc(-c2nc(-c3ccc(-c4ccccc4-c4cccc5c4-c4cc6ccccc6cc4C54C5CC6CC(C5)CC4C6)cc3)cc(-c3ccccc3-c3ccccc3)n2)cc1 ; c1ccc(-c2nc(-c3ccc4cc(-c5cccc6c5-c5cc7ccccc7cc5C65C6CC7CC(C6)CC5C7)ccc4c3)cc(-c3ccccc3-c3ccccc3)n2)cc1]

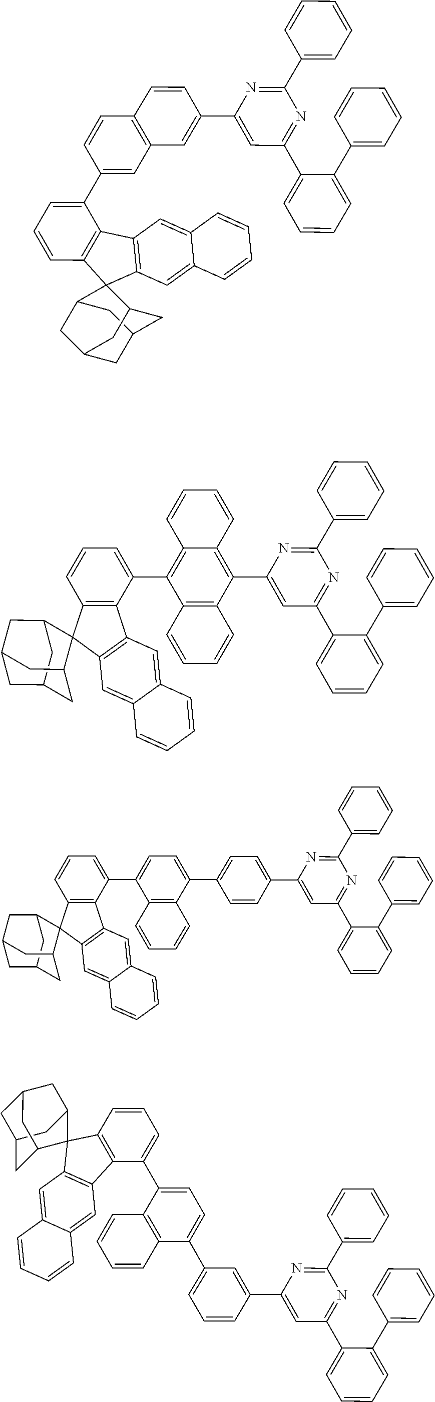 [SMILES: c1ccc(-c2nc(-c3ccc4ccc(-c5cccc6c5-c5cc7ccccc7cc5C65C6CC7CC(C6)CC5C7)cc4c3)cc(-c3ccccc3-c3ccccc3)n2)cc1 ; c1ccc(-c2nc(-c3ccccc3-c3ccccc3)cc(-c3c4ccccc4c(-c4cccc5c4-c4cc6ccccc6cc4C54C5CC6CC(C5)CC4C6)c4ccccc34)n2)cc1 ; c1ccc(-c2nc(-c3ccc(-c4ccc(-c5cccc6c5-c5cc7ccccc7cc5C65C6CC7CC(C6)CC5C7)c5ccccc45)cc3)cc(-c3ccccc3-c3ccccc3)n2)cc1 ; c1ccc(-c2nc(-c3cccc(-c4ccc(-c5cccc6c5-c5cc7ccccc7cc5C65C6CC7CC(C6)CC5C7)c5ccccc45)c3)cc(-c3ccccc3-c3ccccc3)n2)cc1]

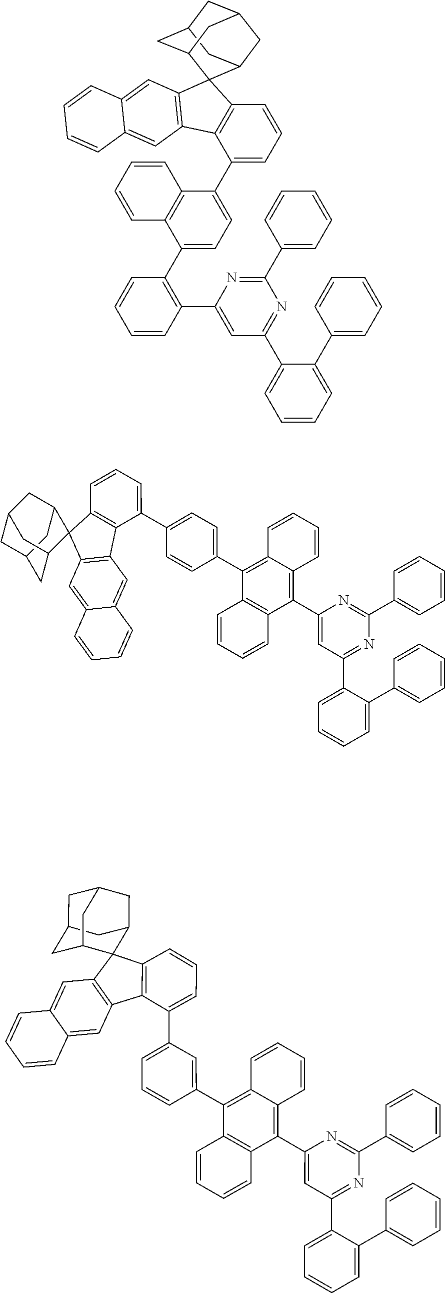 [SMILES: c1ccc(-c2nc(-c3ccccc3-c3ccccc3)cc(-c3ccccc3-c3ccc(-c4cccc5c4-c4cc6ccccc6cc4C54C5CC6CC(C5)CC4C6)c4ccccc34)n2)cc1 ; c1ccc(-c2nc(-c3ccccc3-c3ccccc3)cc(-c3c4ccccc4c(-c4ccc(-c5cccc6c5-c5cc7ccccc7cc5C65C6CC7CC(C6)CC5C7)cc4)c4ccccc34)n2)cc1 ; c1ccc(-c2nc(-c3ccccc3-c3ccccc3)cc(-c3c4ccccc4c(-c4cccc(-c5cccc6c5-c5cc7ccccc7cc5C65C6CC7CC(C6)CC5C7)c4)c4ccccc34)n2)cc1]

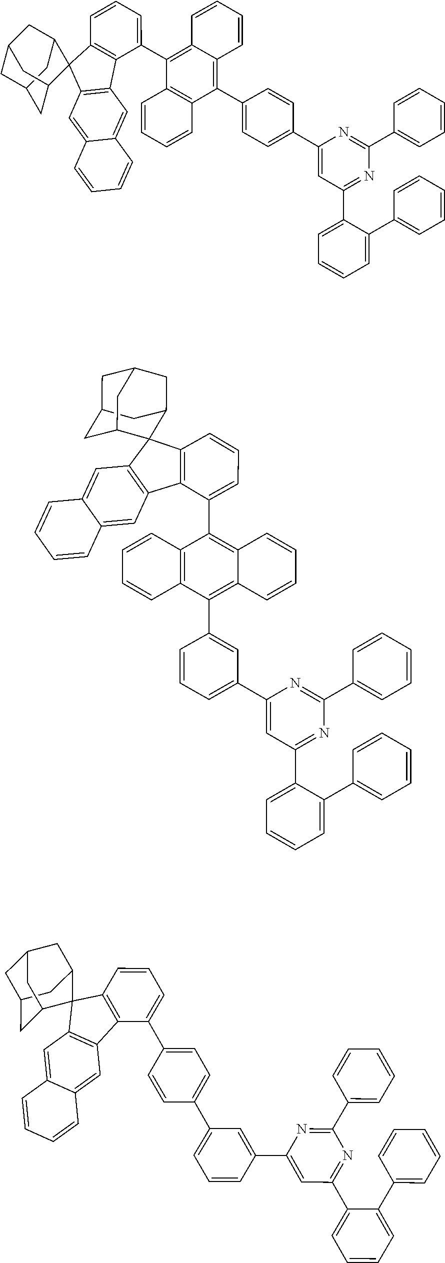 [SMILES: c1ccc(-c2nc(-c3ccc(-c4c5ccccc5c(-c5cccc6c5-c5cc7ccccc7cc5C65C6CC7CC(C6)CC5C7)c5ccccc45)cc3)cc(-c3ccccc3-c3ccccc3)n2)cc1 ; c1ccc(-c2nc(-c3cccc(-c4c5ccccc5c(-c5cccc6c5-c5cc7ccccc7cc5C65C6CC7CC(C6)CC5C7)c5ccccc45)c3)cc(-c3ccccc3-c3ccccc3)n2)cc1 ; c1ccc(-c2nc(-c3cccc(-c4ccc(-c5cccc6c5-c5cc7ccccc7cc5C65C6CC7CC(C6)CC5C7)cc4)c3)cc(-c3ccccc3-c3ccccc3)n2)cc1]

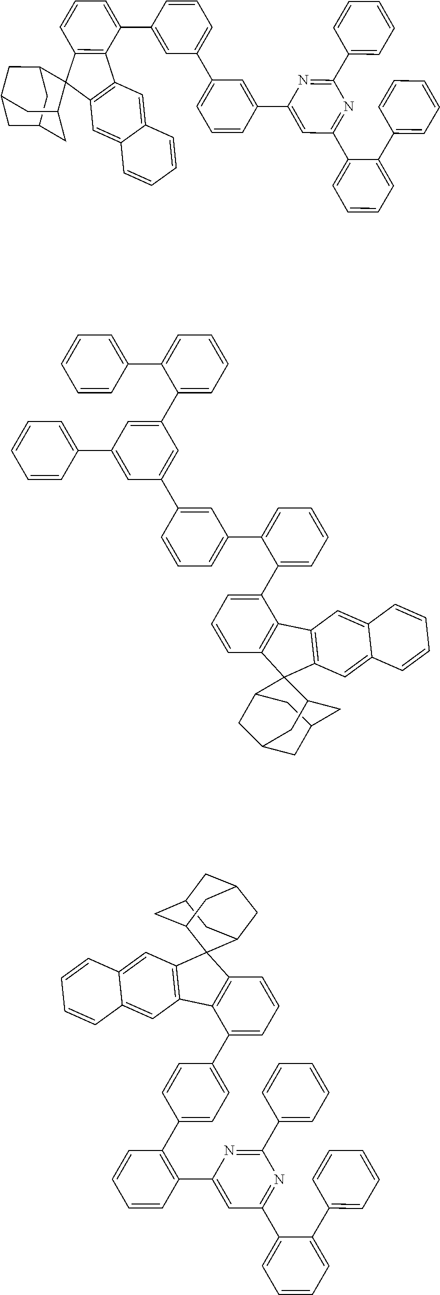

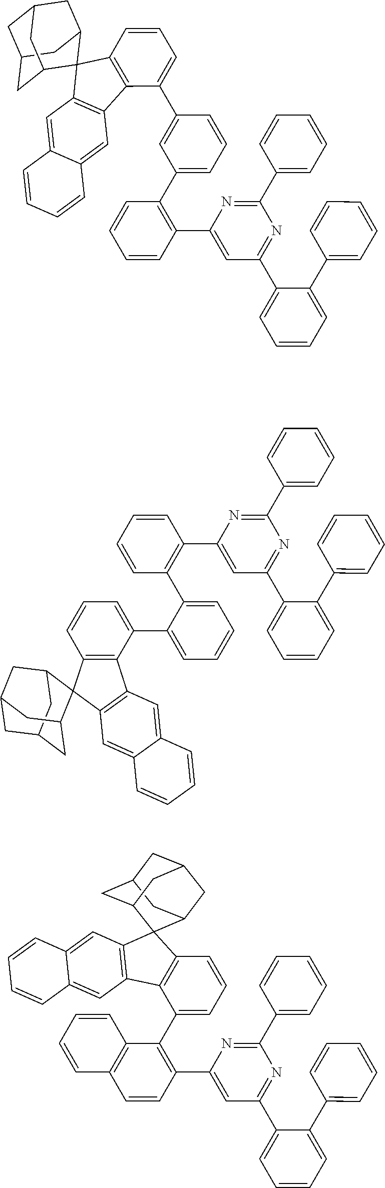 [SMILES: c1ccc(-c2nc(-c3ccccc3-c3ccccc3)cc(-c3ccccc3-c3cccc(-c4cccc5c4-c4cc6ccccc6cc4C54C5CC6CC(C5)CC4C6)c3)n2)cc1 ; c1ccc(-c2nc(-c3ccccc3-c3ccccc3)cc(-c3ccccc3-c3ccccc3-c3cccc4c3-c3cc5ccccc5cc3C43C4CC5CC(C4)CC3C5)n2)cc1 ; c1ccc(-c2nc(-c3ccccc3-c3ccccc3)cc(-c3ccc4ccccc4c3-c3cccc4c3-c3cc5ccccc5cc3C43C4CC5CC(C4)CC3C5)n2)cc1]

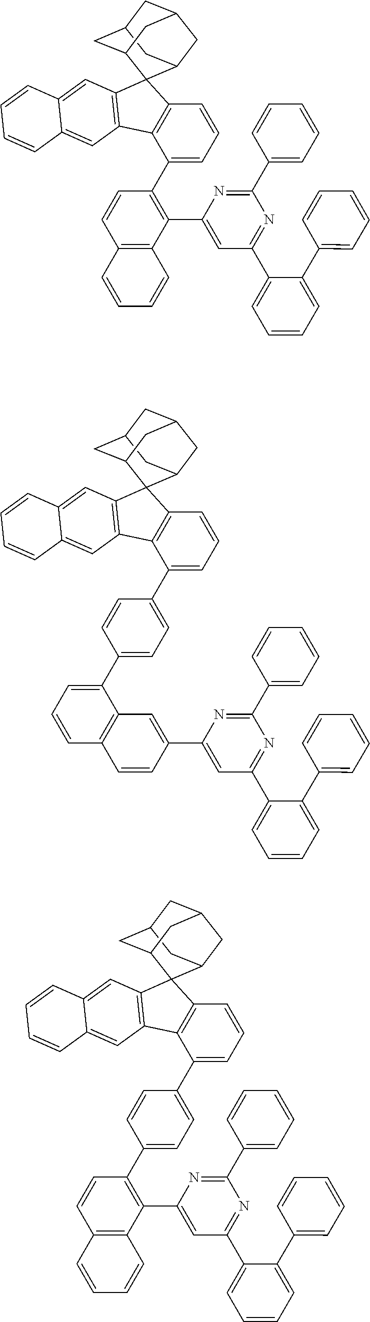 [SMILES: c1ccc(-c2nc(-c3ccccc3-c3ccccc3)cc(-c3c(-c4cccc5c4-c4cc6ccccc6cc4C54C5CC6CC(C5)CC4C6)ccc4ccccc34)n2)cc1 ; c1ccc(-c2nc(-c3ccc4cccc(-c5ccc(-c6cccc7c6-c6cc8ccccc8cc6C76C7CC8CC(C7)CC6C8)cc5)c4c3)cc(-c3ccccc3-c3ccccc3)n2)cc1 ; c1ccc(-c2nc(-c3ccccc3-c3ccccc3)cc(-c3c(-c4ccc(-c5cccc6c5-c5cc7ccccc7cc5C65C6CC7CC(C6)CC5C7)cc4)ccc4ccccc34)n2)cc1]

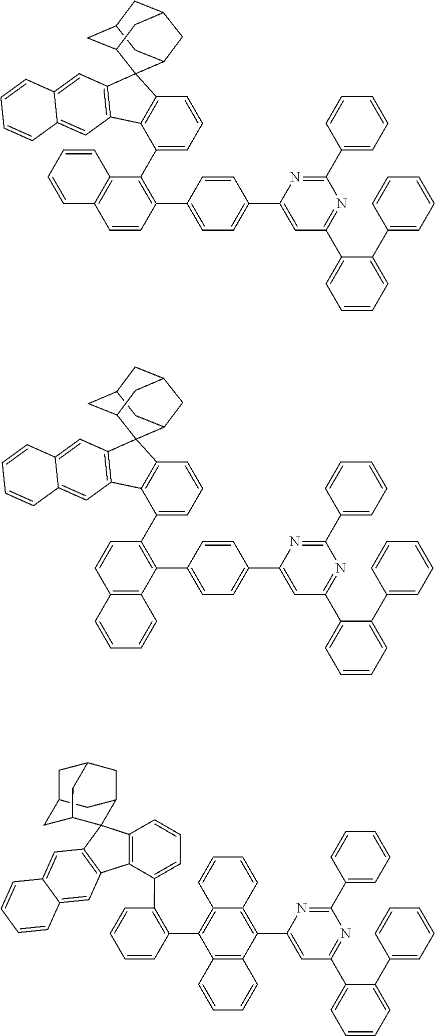 [SMILES: c1ccc(-c2nc(-c3ccc(-c4ccc5ccccc5c4-c4cccc5c4-c4cc6ccccc6cc4C54C5CC6CC(C5)CC4C6)cc3)cc(-c3ccccc3-c3ccccc3)n2)cc1 ; c1ccc(-c2nc(-c3ccc(-c4c(-c5cccc6c5-c5cc7ccccc7cc5C65C6CC7CC(C6)CC5C7)ccc5ccccc45)cc3)cc(-c3ccccc3-c3ccccc3)n2)cc1 ; c1ccc(-c2nc(-c3ccccc3-c3ccccc3)cc(-c3c4ccccc4c(-c4ccccc4-c4cccc5c4-c4cc6ccccc6cc4C54C5CC6CC(C5)CC4C6)c4ccccc34)n2)cc1]

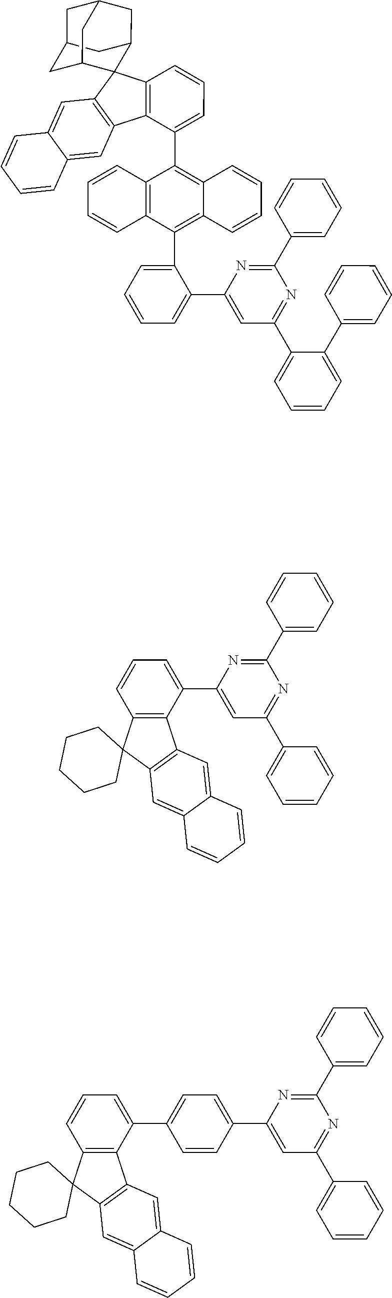 [SMILES: c1ccc(-c2nc(-c3ccccc3-c3ccccc3)cc(-c3ccccc3-c3c4ccccc4c(-c4cccc5c4-c4cc6ccccc6cc4C54C5CC6CC(C5)CC4C6)c4ccccc34)n2)cc1 ; c1ccc(-c2cc(-c3cccc4c3-c3cc5ccccc5cc3C43CCCCC3)nc(-c3ccccc3)n2)cc1 ; c1ccc(-c2cc(-c3ccc(-c4cccc5c4-c4cc6ccccc6cc4C54CCCCC4)cc3)nc(-c3ccccc3)n2)cc1]

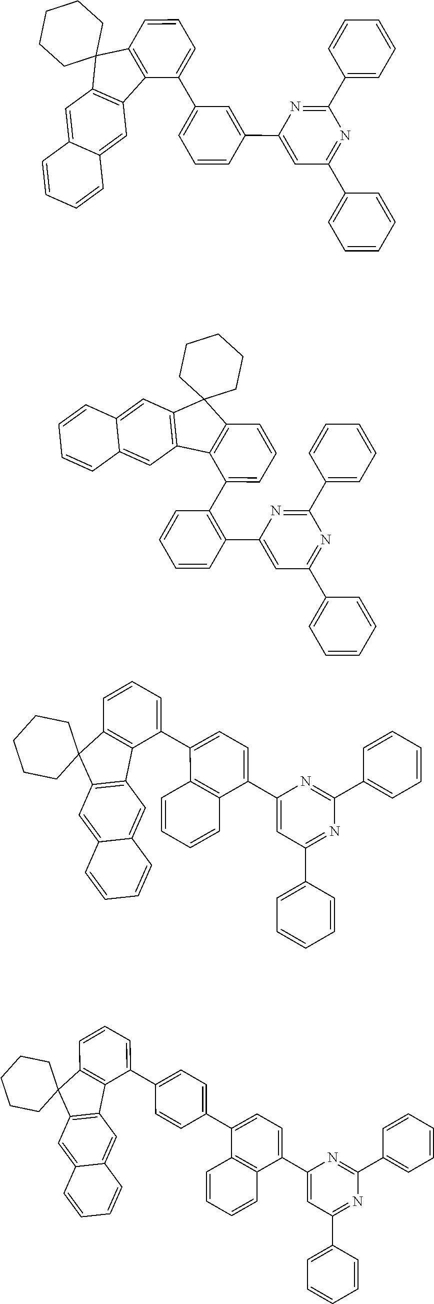 [SMILES: c1ccc(-c2cc(-c3cccc(-c4cccc5c4-c4cc6ccccc6cc4C54CCCCC4)c3)nc(-c3ccccc3)n2)cc1 ; c1ccc(-c2cc(-c3ccccc3-c3cccc4c3-c3cc5ccccc5cc3C43CCCCC3)nc(-c3ccccc3)n2)cc1 ; c1ccc(-c2cc(-c3ccc(-c4cccc5c4-c4cc6ccccc6cc4C54CCCCC4)c4ccccc34)nc(-c3ccccc3)n2)cc1 ; c1ccc(-c2cc(-c3ccc(-c4ccc(-c5cccc6c5-c5cc7ccccc7cc5C65CCCCC5)cc4)c4ccccc34)nc(-c3ccccc3)n2)cc1]

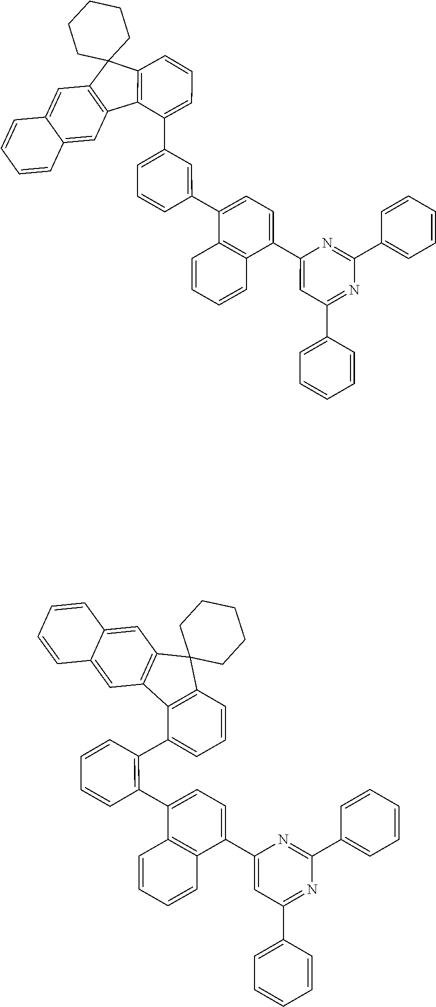 [SMILES: c1ccc(-c2cc(-c3ccc(-c4cccc(-c5cccc6c5-c5cc7ccccc7cc5C65CCCCC5)c4)c4ccccc34)nc(-c3ccccc3)n2)cc1 ; c1ccc(-c2cc(-c3ccc(-c4ccccc4-c4cccc5c4-c4cc6ccccc6cc4C54CCCCC4)c4ccccc34)nc(-c3ccccc3)n2)cc1]

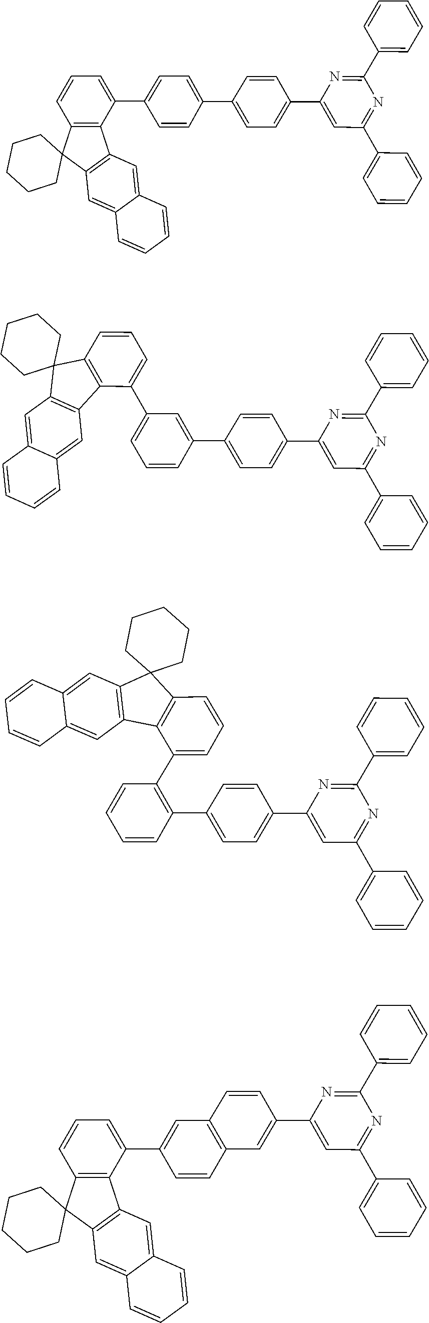 [SMILES: c1ccc(-c2cc(-c3ccc(-c4ccc(-c5cccc6c5-c5cc7ccccc7cc5C65CCCCC5)cc4)cc3)nc(-c3ccccc3)n2)cc1 ; c1ccc(-c2cc(-c3ccc(-c4cccc(-c5cccc6c5-c5cc7ccccc7cc5C65CCCCC5)c4)cc3)nc(-c3ccccc3)n2)cc1 ; c1ccc(-c2cc(-c3ccc(-c4ccccc4-c4cccc5c4-c4cc6ccccc6cc4C54CCCCC4)cc3)nc(-c3ccccc3)n2)cc1 ; c1ccc(-c2cc(-c3ccc4cc(-c5cccc6c5-c5cc7ccccc7cc5C65CCCCC5)ccc4c3)nc(-c3ccccc3)n2)cc1]

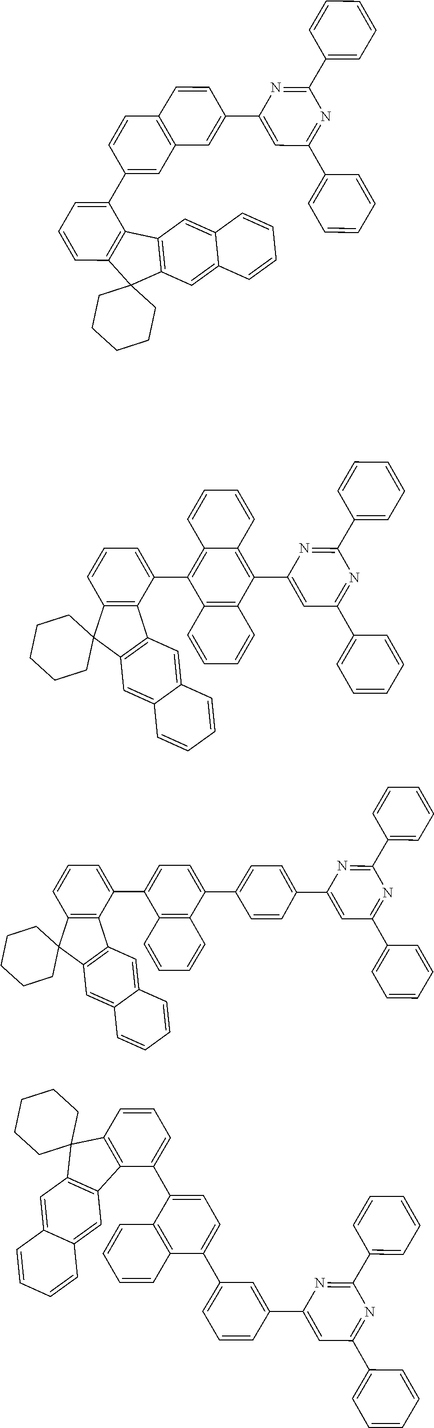 [SMILES: c1ccc(-c2cc(-c3ccc4ccc(-c5cccc6c5-c5cc7ccccc7cc5C65CCCCC5)cc4c3)nc(-c3ccccc3)n2)cc1 ; c1ccc(-c2cc(-c3c4ccccc4c(-c4cccc5c4-c4cc6ccccc6cc4C54CCCCC4)c4ccccc34)nc(-c3ccccc3)n2)cc1 ; c1ccc(-c2cc(-c3ccc(-c4ccc(-c5cccc6c5-c5cc7ccccc7cc5C65CCCCC5)c5ccccc45)cc3)nc(-c3ccccc3)n2)cc1 ; c1ccc(-c2cc(-c3cccc(-c4ccc(-c5cccc6c5-c5cc7ccccc7cc5C65CCCCC5)c5ccccc45)c3)nc(-c3ccccc3)n2)cc1]

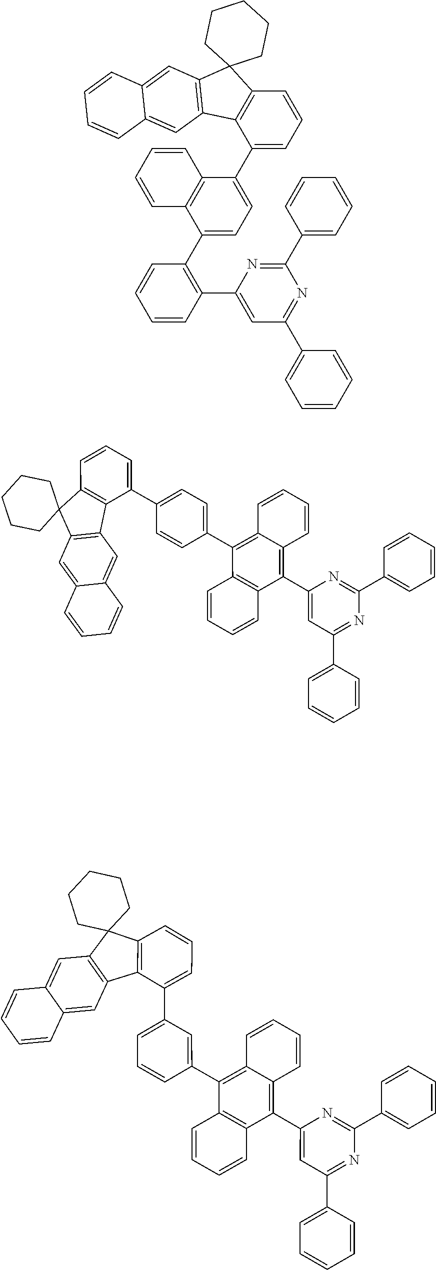 [SMILES: c1ccc(-c2cc(-c3ccccc3-c3ccc(-c4cccc5c4-c4cc6ccccc6cc4C54CCCCC4)c4ccccc34)nc(-c3ccccc3)n2)cc1 ; c1ccc(-c2cc(-c3c4ccccc4c(-c4ccc(-c5cccc6c5-c5cc7ccccc7cc5C65CCCCC5)cc4)c4ccccc34)nc(-c3ccccc3)n2)cc1 ; c1ccc(-c2cc(-c3c4ccccc4c(-c4cccc(-c5cccc6c5-c5cc7ccccc7cc5C65CCCCC5)c4)c4ccccc34)nc(-c3ccccc3)n2)cc1]

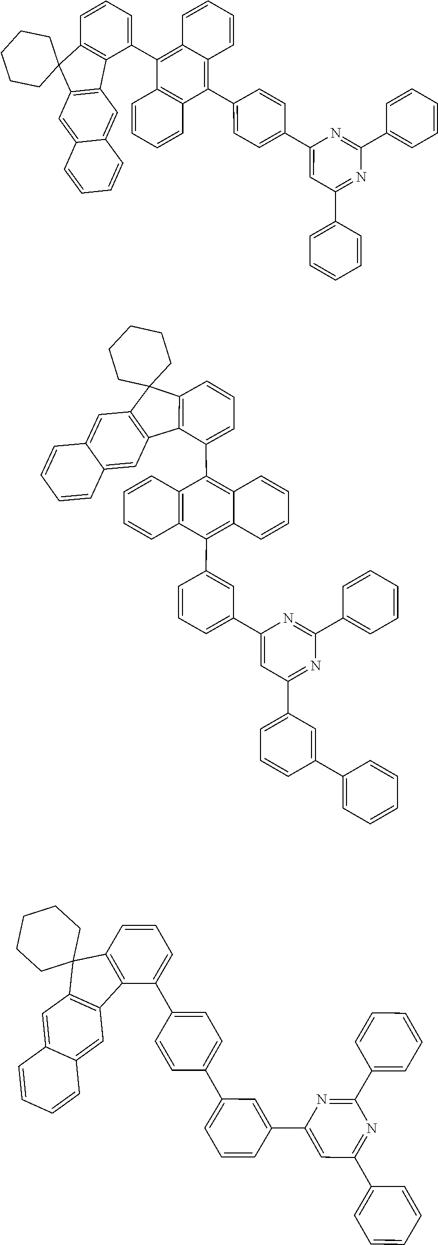 [SMILES: c1ccc(-c2cc(-c3ccc(-c4c5ccccc5c(-c5cccc6c5-c5cc7ccccc7cc5C65CCCCC5)c5ccccc45)cc3)nc(-c3ccccc3)n2)cc1 ; c1ccc(-c2cccc(-c3cc(-c4cccc(-c5c6ccccc6c(-c6cccc7c6-c6cc8ccccc8cc6C76CCCCC6)c6ccccc56)c4)nc(-c4ccccc4)n3)c2)cc1 ; c1ccc(-c2cc(-c3cccc(-c4ccc(-c5cccc6c5-c5cc7ccccc7cc5C65CCCCC5)cc4)c3)nc(-c3ccccc3)n2)cc1]

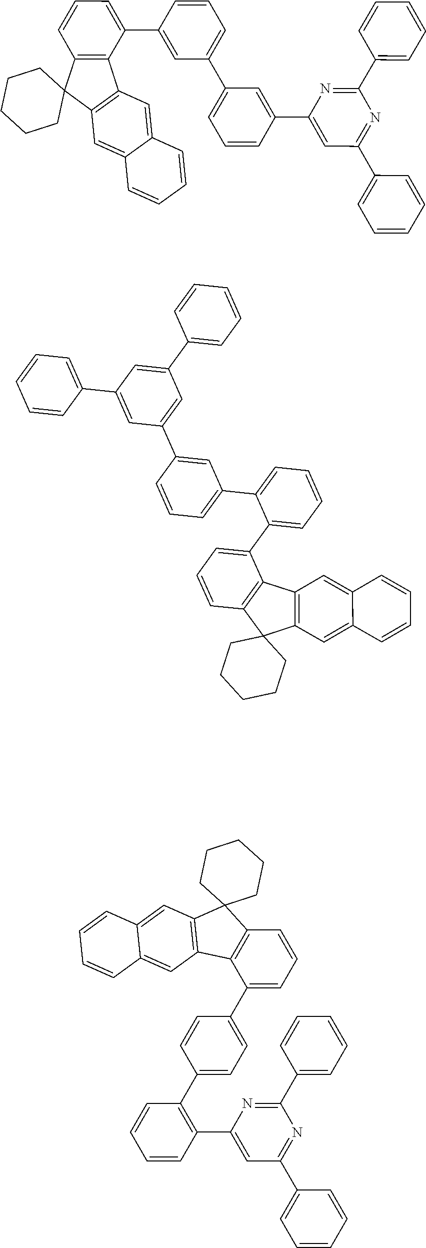

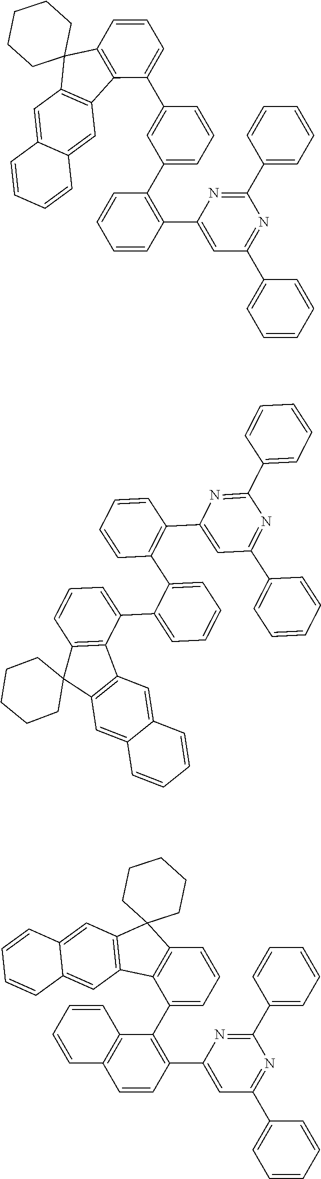 [SMILES: c1ccc(-c2cc(-c3ccccc3-c3cccc(-c4cccc5c4-c4cc6ccccc6cc4C54CCCCC4)c3)nc(-c3ccccc3)n2)cc1 ; c1ccc(-c2cc(-c3ccccc3-c3ccccc3-c3cccc4c3-c3cc5ccccc5cc3C43CCCCC3)nc(-c3ccccc3)n2)cc1 ; c1ccc(-c2cc(-c3ccc4ccccc4c3-c3cccc4c3-c3cc5ccccc5cc3C43CCCCC3)nc(-c3ccccc3)n2)cc1]

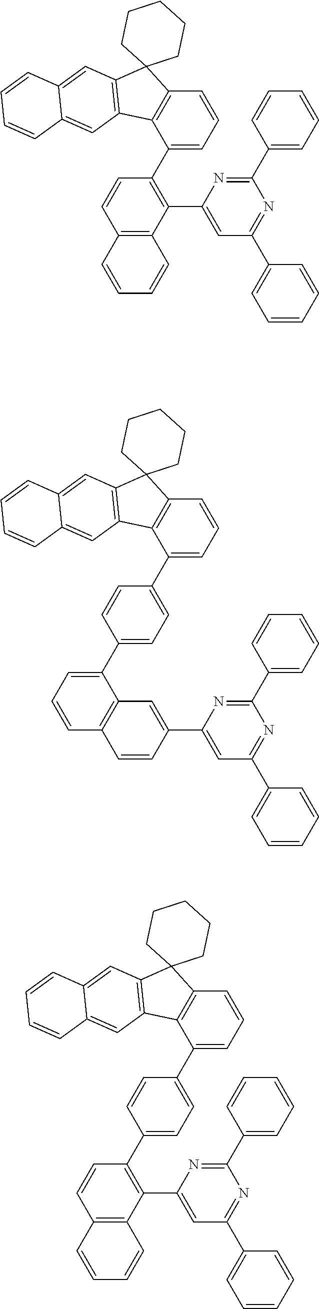 [SMILES: c1ccc(-c2cc(-c3c(-c4cccc5c4-c4cc6ccccc6cc4C54CCCCC4)ccc4ccccc34)nc(-c3ccccc3)n2)cc1 ; c1ccc(-c2cc(-c3ccc4cccc(-c5ccc(-c6cccc7c6-c6cc8ccccc8cc6C76CCCCC6)cc5)c4c3)nc(-c3ccccc3)n2)cc1 ; c1ccc(-c2cc(-c3c(-c4ccc(-c5cccc6c5-c5cc7ccccc7cc5C65CCCCC5)cc4)ccc4ccccc34)nc(-c3ccccc3)n2)cc1]

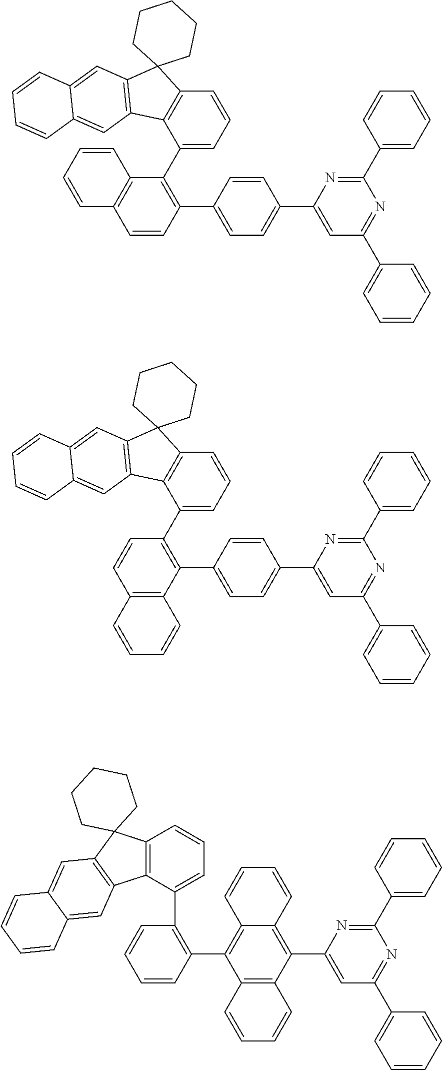 [SMILES: c1ccc(-c2cc(-c3ccc(-c4ccc5ccccc5c4-c4cccc5c4-c4cc6ccccc6cc4C54CCCCC4)cc3)nc(-c3ccccc3)n2)cc1 ; c1ccc(-c2cc(-c3ccc(-c4c(-c5cccc6c5-c5cc7ccccc7cc5C65CCCCC5)ccc5ccccc45)cc3)nc(-c3ccccc3)n2)cc1 ; c1ccc(-c2cc(-c3c4ccccc4c(-c4ccccc4-c4cccc5c4-c4cc6ccccc6cc4C54CCCCC4)c4ccccc34)nc(-c3ccccc3)n2)cc1]

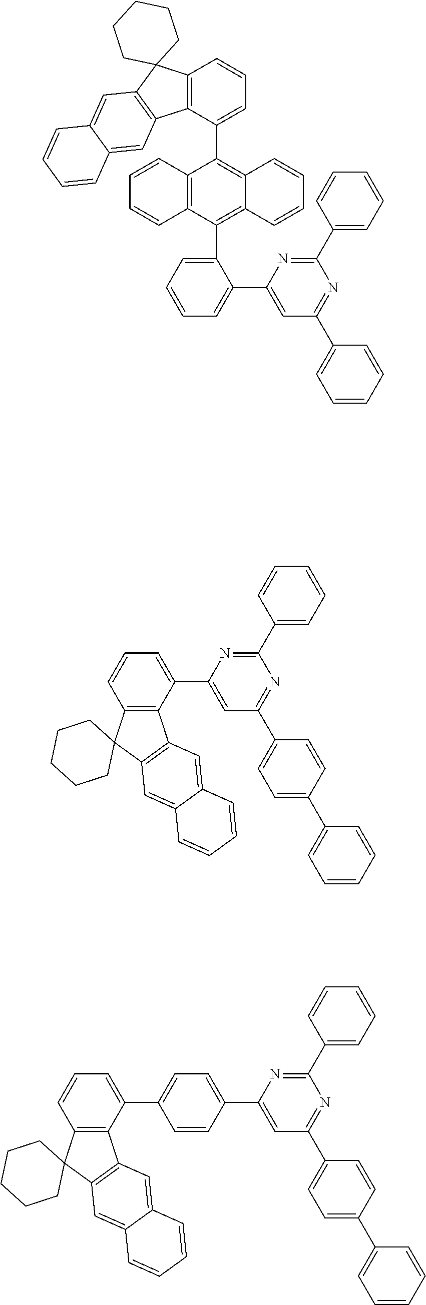 [SMILES: c1ccc(-c2cc(-c3ccccc3-c3c4ccccc4c(-c4cccc5c4-c4cc6ccccc6cc4C54CCCCC4)c4ccccc34)nc(-c3ccccc3)n2)cc1 ; c1ccc(-c2ccc(-c3cc(-c4cccc5c4-c4cc6ccccc6cc4C54CCCCC4)nc(-c4ccccc4)n3)cc2)cc1 ; c1ccc(-c2ccc(-c3cc(-c4ccc(-c5cccc6c5-c5cc7ccccc7cc5C65CCCCC5)cc4)nc(-c4ccccc4)n3)cc2)cc1]

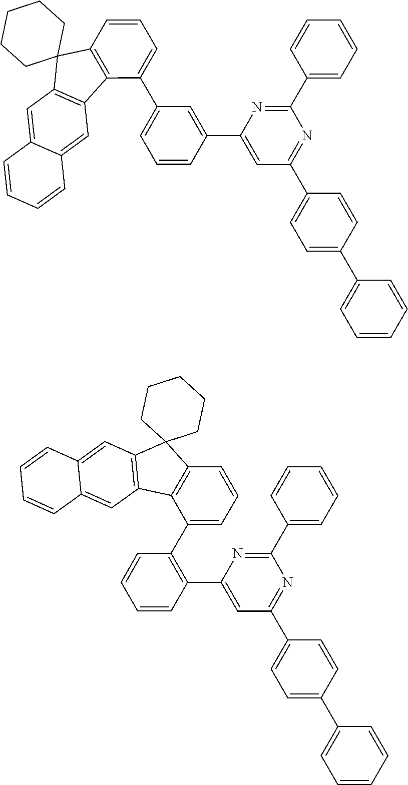 [SMILES: c1ccc(-c2ccc(-c3cc(-c4cccc(-c5cccc6c5-c5cc7ccccc7cc5C65CCCCC5)c4)nc(-c4ccccc4)n3)cc2)cc1 ; c1ccc(-c2ccc(-c3cc(-c4ccccc4-c4cccc5c4-c4cc6ccccc6cc4C54CCCCC4)nc(-c4ccccc4)n3)cc2)cc1]

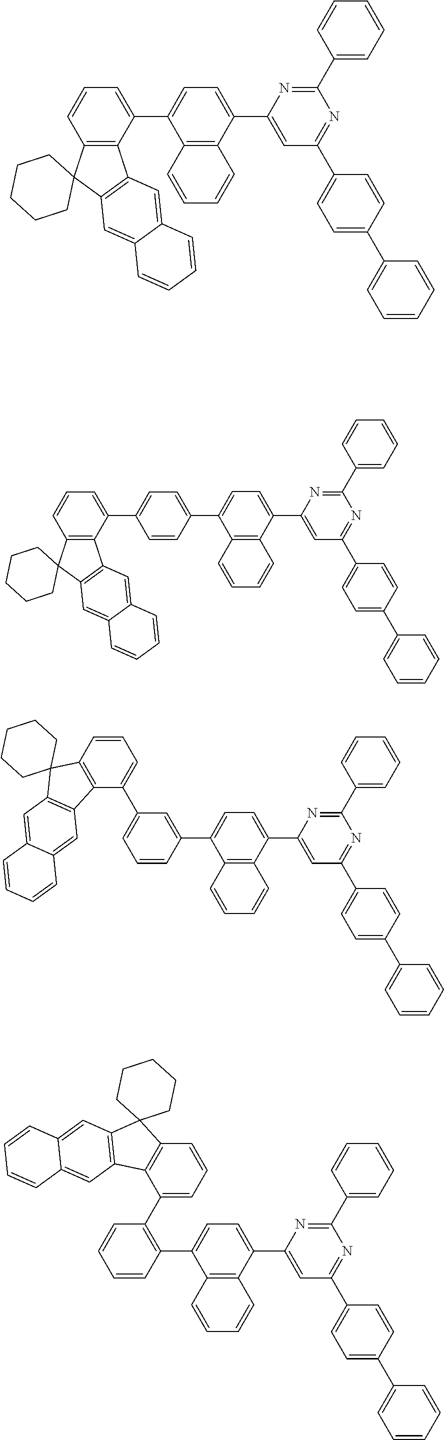 [SMILES: c1ccc(-c2ccc(-c3cc(-c4ccc(-c5cccc6c5-c5cc7ccccc7cc5C65CCCCC5)c5ccccc45)nc(-c4ccccc4)n3)cc2)cc1 ; c1ccc(-c2ccc(-c3cc(-c4ccc(-c5ccc(-c6cccc7c6-c6cc8ccccc8cc6C76CCCCC6)cc5)c5ccccc45)nc(-c4ccccc4)n3)cc2)cc1 ; c1ccc(-c2ccc(-c3cc(-c4ccc(-c5cccc(-c6cccc7c6-c6cc8ccccc8cc6C76CCCCC6)c5)c5ccccc45)nc(-c4ccccc4)n3)cc2)cc1 ; c1ccc(-c2ccc(-c3cc(-c4ccc(-c5ccccc5-c5cccc6c5-c5cc7ccccc7cc5C65CCCCC5)c5ccccc45)nc(-c4ccccc4)n3)cc2)cc1]

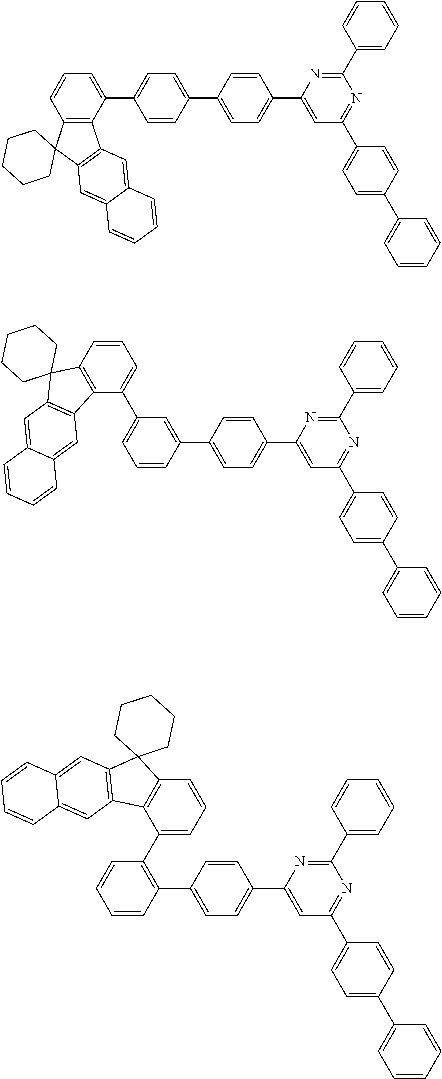 [SMILES: c1ccc(-c2ccc(-c3cc(-c4ccc(-c5ccc(-c6cccc7c6-c6cc8ccccc8cc6C76CCCCC6)cc5)cc4)nc(-c4ccccc4)n3)cc2)cc1 ; c1ccc(-c2ccc(-c3cc(-c4ccc(-c5cccc(-c6cccc7c6-c6cc8ccccc8cc6C76CCCCC6)c5)cc4)nc(-c4ccccc4)n3)cc2)cc1 ; c1ccc(-c2ccc(-c3cc(-c4ccc(-c5ccccc5-c5cccc6c5-c5cc7ccccc7cc5C65CCCCC5)cc4)nc(-c4ccccc4)n3)cc2)cc1]

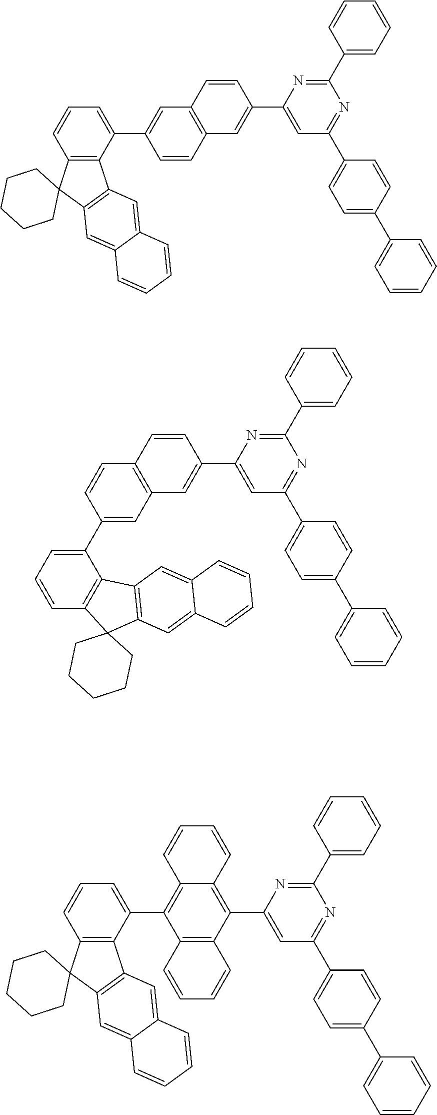 [SMILES: c1ccc(-c2ccc(-c3cc(-c4ccc5cc(-c6cccc7c6-c6cc8ccccc8cc6C76CCCCC6)ccc5c4)nc(-c4ccccc4)n3)cc2)cc1 ; c1ccc(-c2ccc(-c3cc(-c4ccc5ccc(-c6cccc7c6-c6cc8ccccc8cc6C76CCCCC6)cc5c4)nc(-c4ccccc4)n3)cc2)cc1 ; c1ccc(-c2ccc(-c3cc(-c4c5ccccc5c(-c5cccc6c5-c5cc7ccccc7cc5C65CCCCC5)c5ccccc45)nc(-c4ccccc4)n3)cc2)cc1]

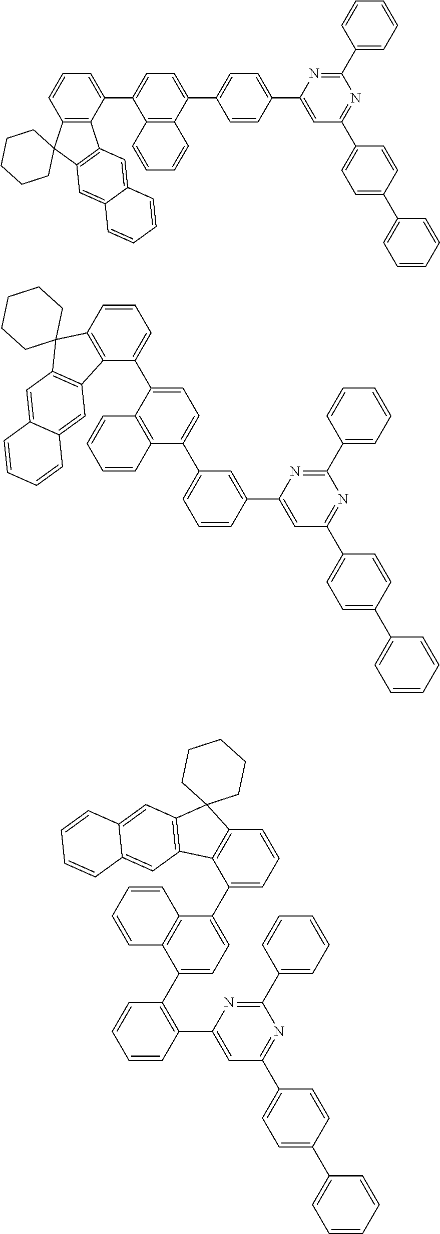 [SMILES: c1ccc(-c2ccc(-c3cc(-c4ccc(-c5ccc(-c6cccc7c6-c6cc8ccccc8cc6C76CCCCC6)c6ccccc56)cc4)nc(-c4ccccc4)n3)cc2)cc1 ; c1ccc(-c2ccc(-c3cc(-c4cccc(-c5ccc(-c6cccc7c6-c6cc8ccccc8cc6C76CCCCC6)c6ccccc56)c4)nc(-c4ccccc4)n3)cc2)cc1 ; c1ccc(-c2ccc(-c3cc(-c4ccccc4-c4ccc(-c5cccc6c5-c5cc7ccccc7cc5C65CCCCC5)c5ccccc45)nc(-c4ccccc4)n3)cc2)cc1]

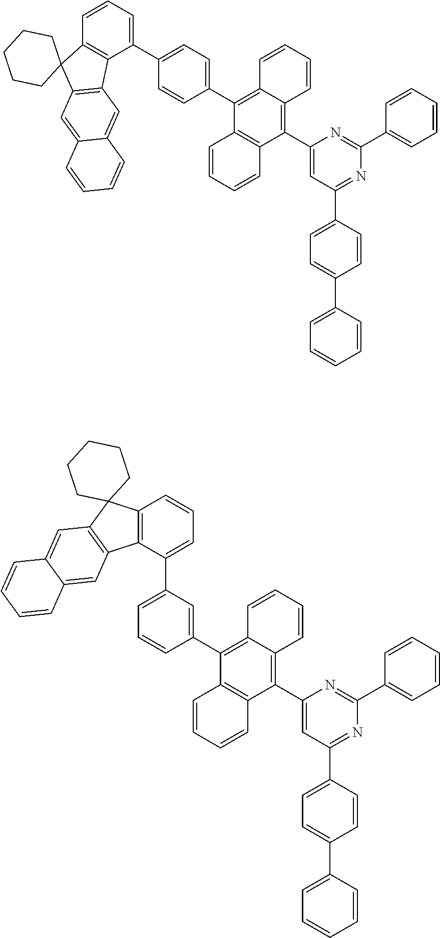 [SMILES: c1ccc(-c2ccc(-c3cc(-c4c5ccccc5c(-c5ccc(-c6cccc7c6-c6cc8ccccc8cc6C76CCCCC6)cc5)c5ccccc45)nc(-c4ccccc4)n3)cc2)cc1 ; c1ccc(-c2ccc(-c3cc(-c4c5ccccc5c(-c5cccc(-c6cccc7c6-c6cc8ccccc8cc6C76CCCCC6)c5)c5ccccc45)nc(-c4ccccc4)n3)cc2)cc1]

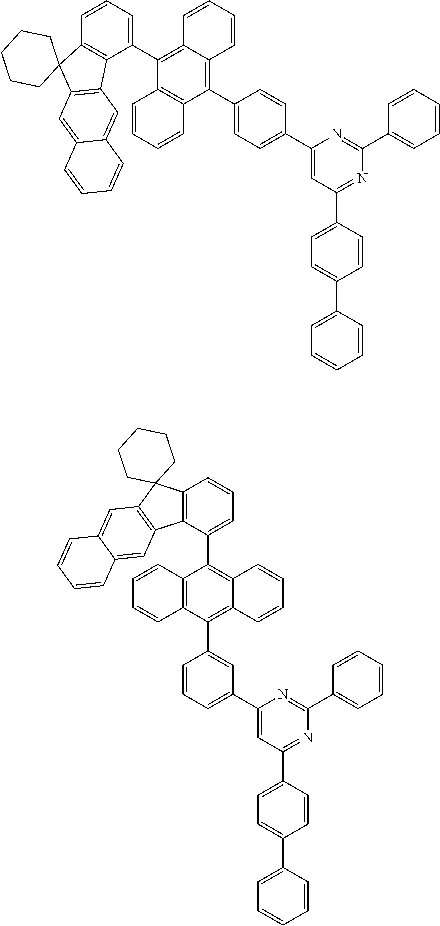 [SMILES: c1ccc(-c2ccc(-c3cc(-c4ccc(-c5c6ccccc6c(-c6cccc7c6-c6cc8ccccc8cc6C76CCCCC6)c6ccccc56)cc4)nc(-c4ccccc4)n3)cc2)cc1 ; c1ccc(-c2ccc(-c3cc(-c4cccc(-c5c6ccccc6c(-c6cccc7c6-c6cc8ccccc8cc6C76CCCCC6)c6ccccc56)c4)nc(-c4ccccc4)n3)cc2)cc1]

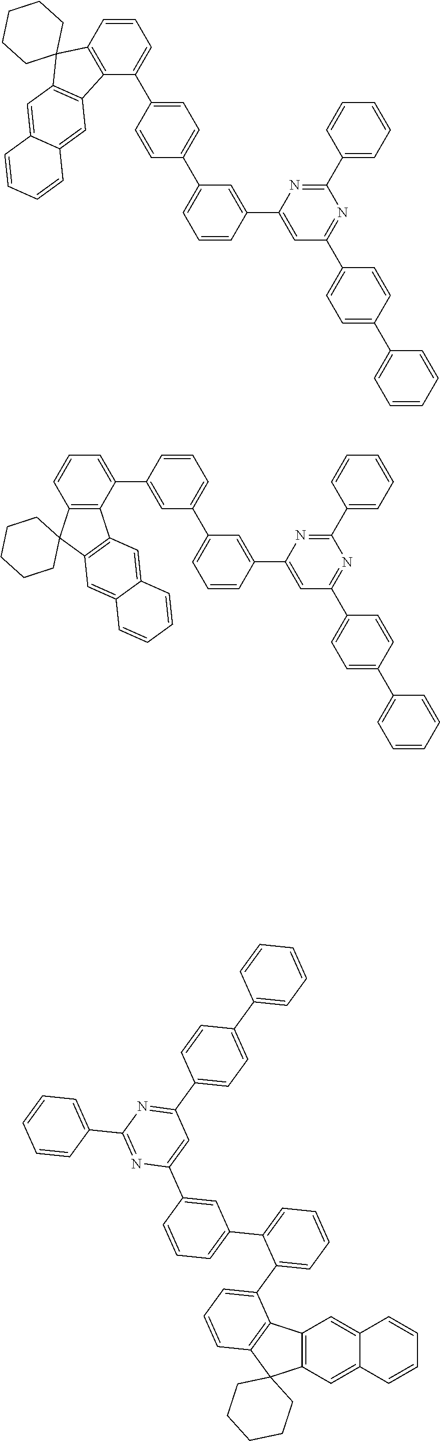 [SMILES: c1ccc(-c2ccc(-c3cc(-c4cccc(-c5ccc(-c6cccc7c6-c6cc8ccccc8cc6C76CCCCC6)cc5)c4)nc(-c4ccccc4)n3)cc2)cc1 ; c1ccc(-c2ccc(-c3cc(-c4cccc(-c5cccc(-c6cccc7c6-c6cc8ccccc8cc6C76CCCCC6)c5)c4)nc(-c4ccccc4)n3)cc2)cc1 ; c1ccc(-c2ccc(-c3cc(-c4cccc(-c5ccccc5-c5cccc6c5-c5cc7ccccc7cc5C65CCCCC5)c4)nc(-c4ccccc4)n3)cc2)cc1]

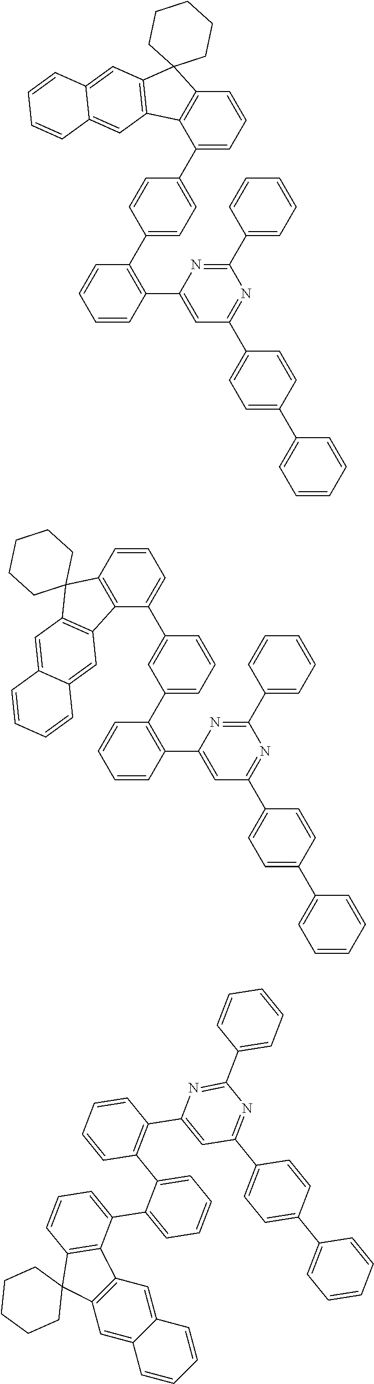 [SMILES: c1ccc(-c2ccc(-c3cc(-c4ccccc4-c4ccc(-c5cccc6c5-c5cc7ccccc7cc5C65CCCCC5)cc4)nc(-c4ccccc4)n3)cc2)cc1 ; c1ccc(-c2ccc(-c3cc(-c4ccccc4-c4cccc(-c5cccc6c5-c5cc7ccccc7cc5C65CCCCC5)c4)nc(-c4ccccc4)n3)cc2)cc1 ; c1ccc(-c2ccc(-c3cc(-c4ccccc4-c4ccccc4-c4cccc5c4-c4cc6ccccc6cc4C54CCCCC4)nc(-c4ccccc4)n3)cc2)cc1]

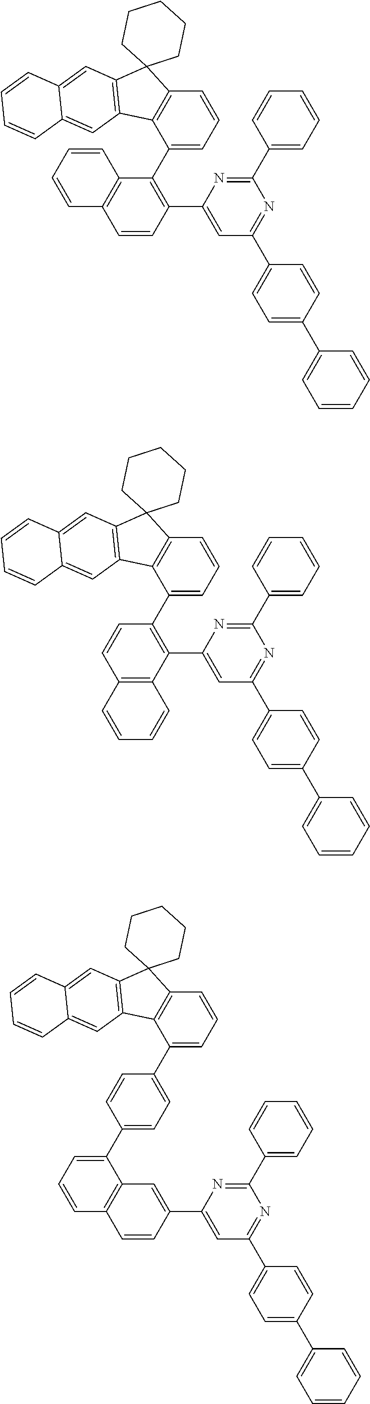 [SMILES: c1ccc(-c2ccc(-c3cc(-c4ccc5ccccc5c4-c4cccc5c4-c4cc6ccccc6cc4C54CCCCC4)nc(-c4ccccc4)n3)cc2)cc1 ; c1ccc(-c2ccc(-c3cc(-c4c(-c5cccc6c5-c5cc7ccccc7cc5C65CCCCC5)ccc5ccccc45)nc(-c4ccccc4)n3)cc2)cc1 ; c1ccc(-c2ccc(-c3cc(-c4ccc5cccc(-c6ccc(-c7cccc8c7-c7cc9ccccc9cc7C87CCCCC7)cc6)c5c4)nc(-c4ccccc4)n3)cc2)cc1]

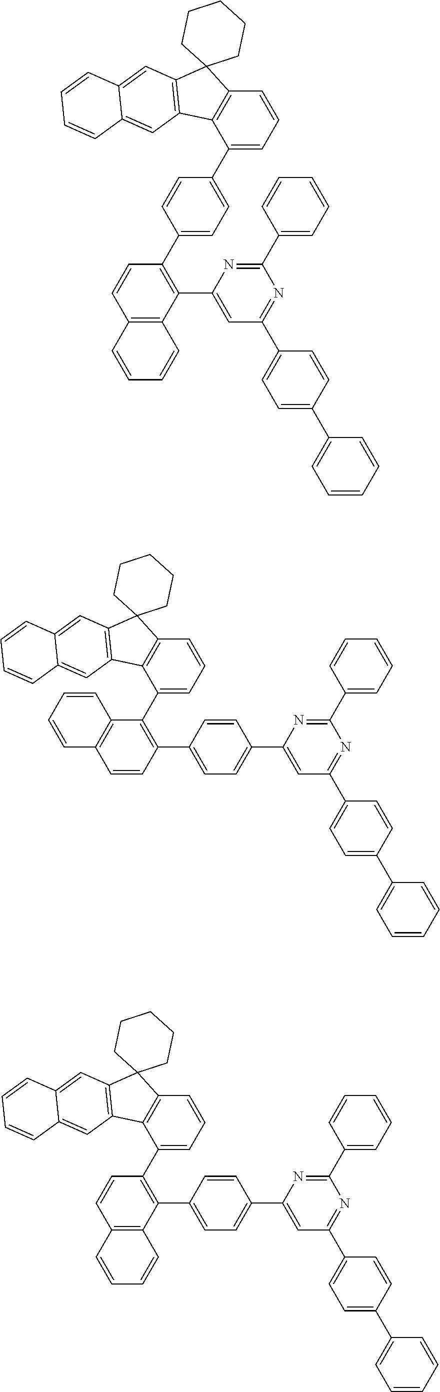 [SMILES: c1ccc(-c2ccc(-c3cc(-c4c(-c5ccc(-c6cccc7c6-c6cc8ccccc8cc6C76CCCCC6)cc5)ccc5ccccc45)nc(-c4ccccc4)n3)cc2)cc1 ; c1ccc(-c2ccc(-c3cc(-c4ccc(-c5ccc6ccccc6c5-c5cccc6c5-c5cc7ccccc7cc5C65CCCCC5)cc4)nc(-c4ccccc4)n3)cc2)cc1 ; c1ccc(-c2ccc(-c3cc(-c4ccc(-c5c(-c6cccc7c6-c6cc8ccccc8cc6C76CCCCC6)ccc6ccccc56)cc4)nc(-c4ccccc4)n3)cc2)cc1]

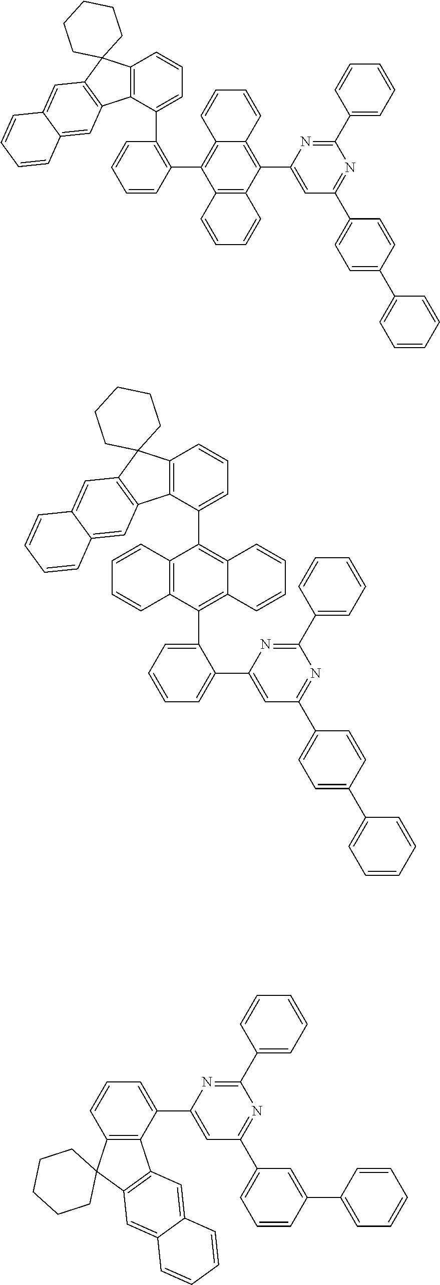 [SMILES: c1ccc(-c2ccc(-c3cc(-c4c5ccccc5c(-c5ccccc5-c5cccc6c5-c5cc7ccccc7cc5C65CCCCC5)c5ccccc45)nc(-c4ccccc4)n3)cc2)cc1 ; c1ccc(-c2ccc(-c3cc(-c4ccccc4-c4c5ccccc5c(-c5cccc6c5-c5cc7ccccc7cc5C65CCCCC5)c5ccccc45)nc(-c4ccccc4)n3)cc2)cc1 ; c1ccc(-c2cccc(-c3cc(-c4cccc5c4-c4cc6ccccc6cc4C54CCCCC4)nc(-c4ccccc4)n3)c2)cc1]

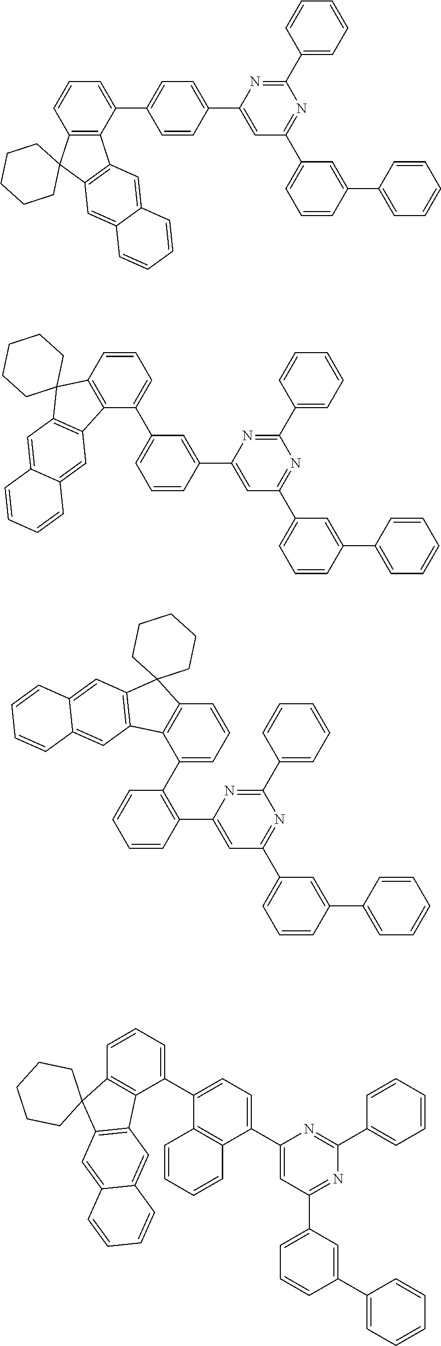 [SMILES: c1ccc(-c2cccc(-c3cc(-c4ccc(-c5cccc6c5-c5cc7ccccc7cc5C65CCCCC5)cc4)nc(-c4ccccc4)n3)c2)cc1 ; c1ccc(-c2cccc(-c3cc(-c4cccc(-c5cccc6c5-c5cc7ccccc7cc5C65CCCCC5)c4)nc(-c4ccccc4)n3)c2)cc1 ; c1ccc(-c2cccc(-c3cc(-c4ccccc4-c4cccc5c4-c4cc6ccccc6cc4C54CCCCC4)nc(-c4ccccc4)n3)c2)cc1 ; c1ccc(-c2cccc(-c3cc(-c4ccc(-c5cccc6c5-c5cc7ccccc7cc5C65CCCCC5)c5ccccc45)nc(-c4ccccc4)n3)c2)cc1]

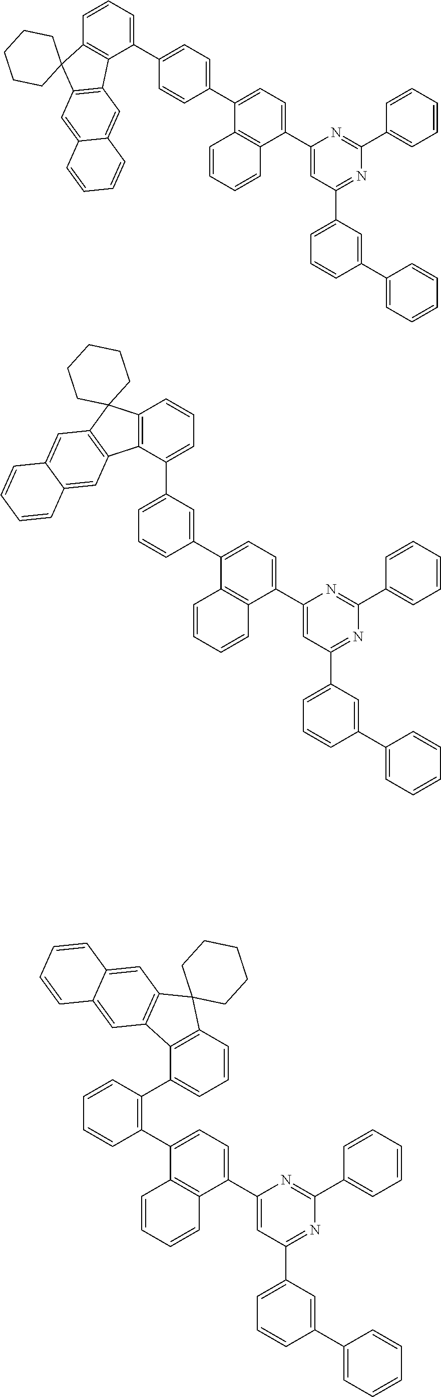 [SMILES: c1ccc(-c2cccc(-c3cc(-c4ccc(-c5ccc(-c6cccc7c6-c6cc8ccccc8cc6C76CCCCC6)cc5)c5ccccc45)nc(-c4ccccc4)n3)c2)cc1 ; c1ccc(-c2cccc(-c3cc(-c4ccc(-c5cccc(-c6cccc7c6-c6cc8ccccc8cc6C76CCCCC6)c5)c5ccccc45)nc(-c4ccccc4)n3)c2)cc1 ; c1ccc(-c2cccc(-c3cc(-c4ccc(-c5ccccc5-c5cccc6c5-c5cc7ccccc7cc5C65CCCCC5)c5ccccc45)nc(-c4ccccc4)n3)c2)cc1]

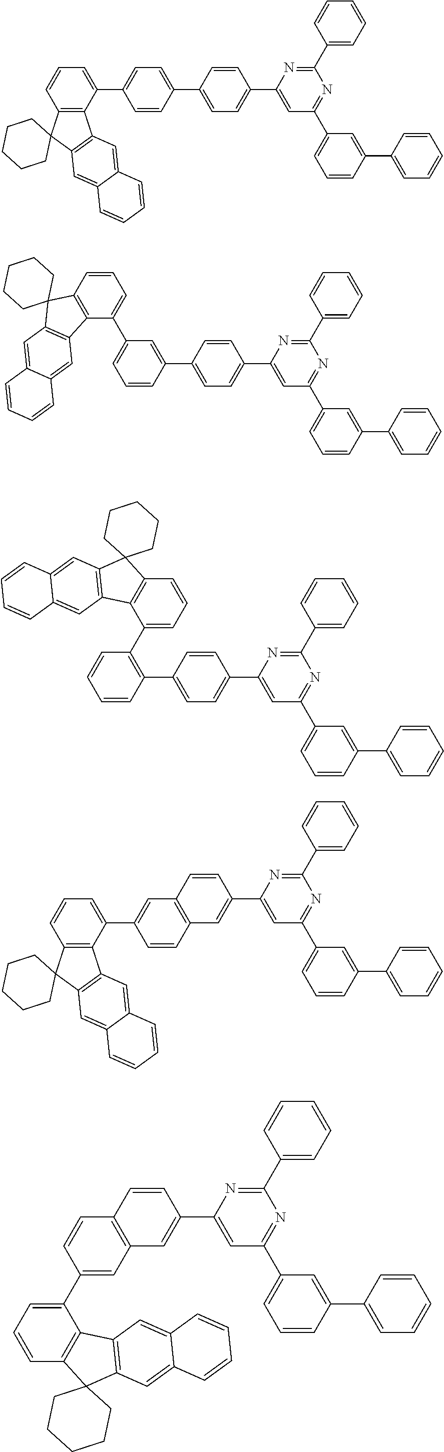

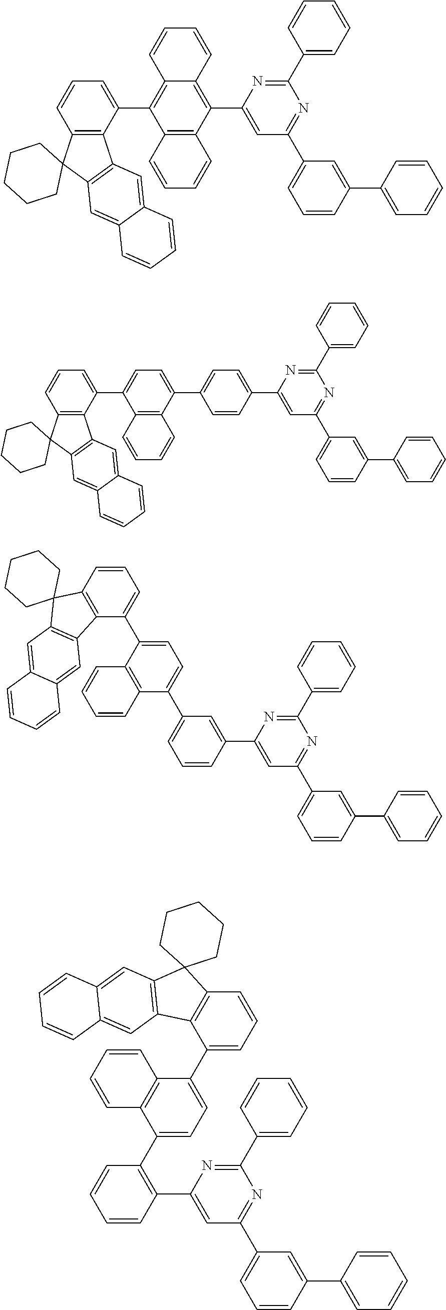 [SMILES: c1ccc(-c2cccc(-c3cc(-c4c5ccccc5c(-c5cccc6c5-c5cc7ccccc7cc5C65CCCCC5)c5ccccc45)nc(-c4ccccc4)n3)c2)cc1 ; c1ccc(-c2cccc(-c3cc(-c4ccc(-c5ccc(-c6cccc7c6-c6cc8ccccc8cc6C76CCCCC6)c6ccccc56)cc4)nc(-c4ccccc4)n3)c2)cc1 ; c1ccc(-c2cccc(-c3cc(-c4cccc(-c5ccc(-c6cccc7c6-c6cc8ccccc8cc6C76CCCCC6)c6ccccc56)c4)nc(-c4ccccc4)n3)c2)cc1 ; c1ccc(-c2cccc(-c3cc(-c4ccccc4-c4ccc(-c5cccc6c5-c5cc7ccccc7cc5C65CCCCC5)c5ccccc45)nc(-c4ccccc4)n3)c2)cc1]

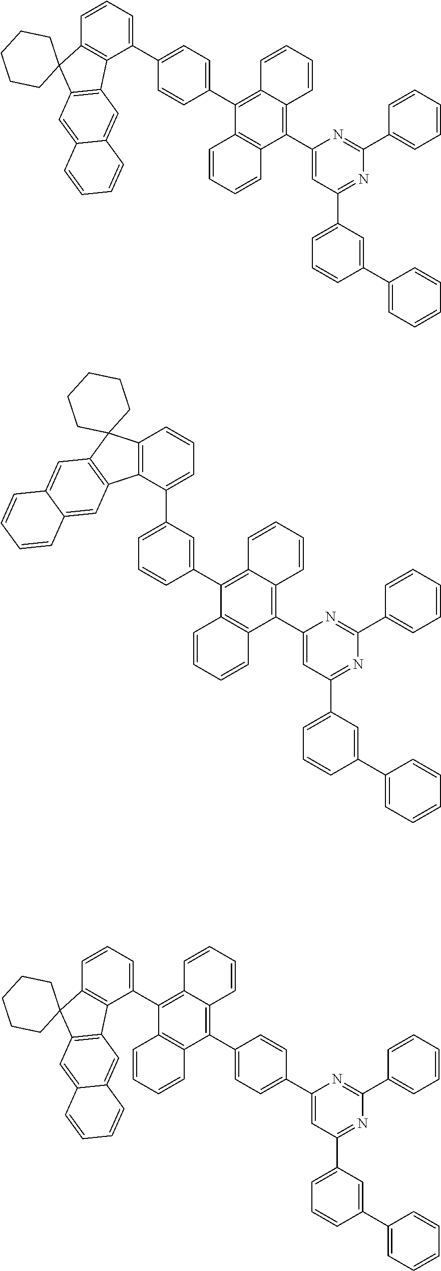 [SMILES: c1ccc(-c2cccc(-c3cc(-c4c5ccccc5c(-c5ccc(-c6cccc7c6-c6cc8ccccc8cc6C76CCCCC6)cc5)c5ccccc45)nc(-c4ccccc4)n3)c2)cc1 ; c1ccc(-c2cccc(-c3cc(-c4c5ccccc5c(-c5cccc(-c6cccc7c6-c6cc8ccccc8cc6C76CCCCC6)c5)c5ccccc45)nc(-c4ccccc4)n3)c2)cc1 ; c1ccc(-c2cccc(-c3cc(-c4ccc(-c5c6ccccc6c(-c6cccc7c6-c6cc8ccccc8cc6C76CCCCC6)c6ccccc56)cc4)nc(-c4ccccc4)n3)c2)cc1]

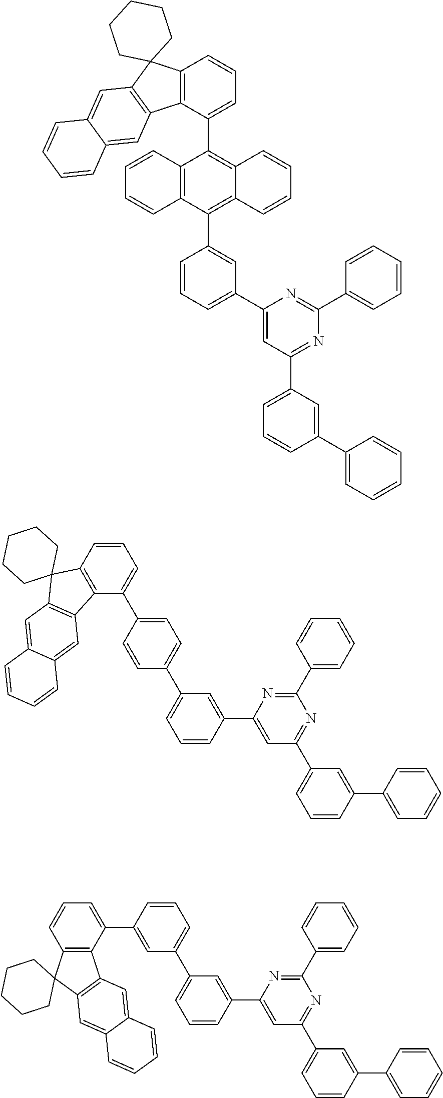 [SMILES: c1ccc(-c2cccc(-c3cc(-c4cccc(-c5c6ccccc6c(-c6cccc7c6-c6cc8ccccc8cc6C76CCCCC6)c6ccccc56)c4)nc(-c4ccccc4)n3)c2)cc1 ; c1ccc(-c2cccc(-c3cc(-c4cccc(-c5ccc(-c6cccc7c6-c6cc8ccccc8cc6C76CCCCC6)cc5)c4)nc(-c4ccccc4)n3)c2)cc1 ; c1ccc(-c2cccc(-c3cc(-c4cccc(-c5cccc(-c6cccc7c6-c6cc8ccccc8cc6C76CCCCC6)c5)c4)nc(-c4ccccc4)n3)c2)cc1]

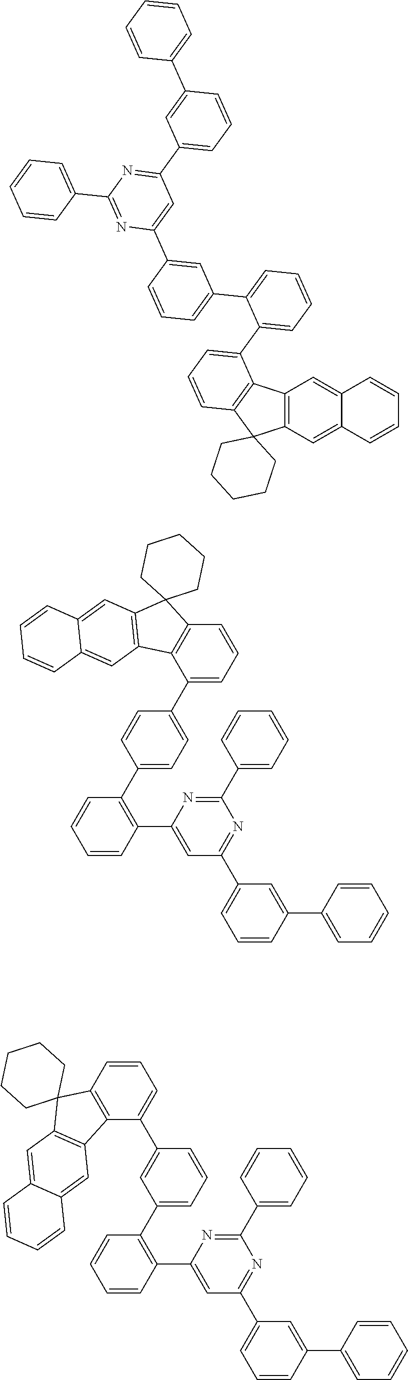 [SMILES: c1ccc(-c2cccc(-c3cc(-c4cccc(-c5ccccc5-c5cccc6c5-c5cc7ccccc7cc5C65CCCCC5)c4)nc(-c4ccccc4)n3)c2)cc1 ; c1ccc(-c2cccc(-c3cc(-c4ccccc4-c4ccc(-c5cccc6c5-c5cc7ccccc7cc5C65CCCCC5)cc4)nc(-c4ccccc4)n3)c2)cc1 ; c1ccc(-c2cccc(-c3cc(-c4ccccc4-c4cccc(-c5cccc6c5-c5cc7ccccc7cc5C65CCCCC5)c4)nc(-c4ccccc4)n3)c2)cc1]

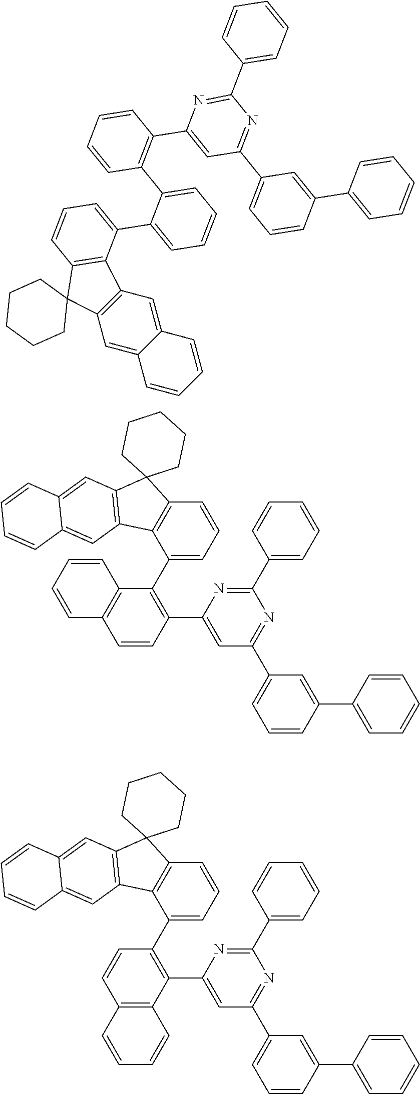

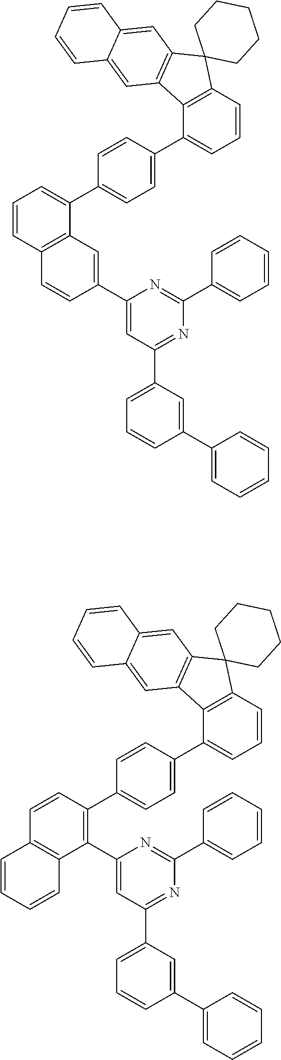 [SMILES: c1ccc(-c2cccc(-c3cc(-c4ccc5cccc(-c6ccc(-c7cccc8c7-c7cc9ccccc9cc7C87CCCCC7)cc6)c5c4)nc(-c4ccccc4)n3)c2)cc1 ; c1ccc(-c2cccc(-c3cc(-c4c(-c5ccc(-c6cccc7c6-c6cc8ccccc8cc6C76CCCCC6)cc5)ccc5ccccc45)nc(-c4ccccc4)n3)c2)cc1]

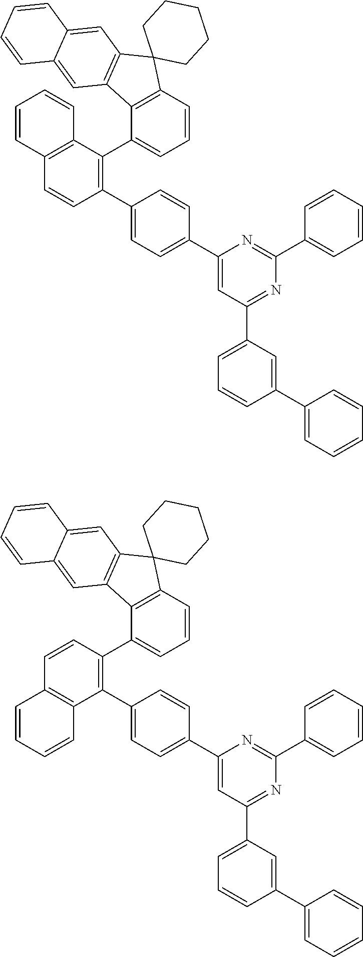 [SMILES: c1ccc(-c2cccc(-c3cc(-c4ccc(-c5ccc6ccccc6c5-c5cccc6c5-c5cc7ccccc7cc5C65CCCCC5)cc4)nc(-c4ccccc4)n3)c2)cc1 ; c1ccc(-c2cccc(-c3cc(-c4ccc(-c5c(-c6cccc7c6-c6cc8ccccc8cc6C76CCCCC6)ccc6ccccc56)cc4)nc(-c4ccccc4)n3)c2)cc1]

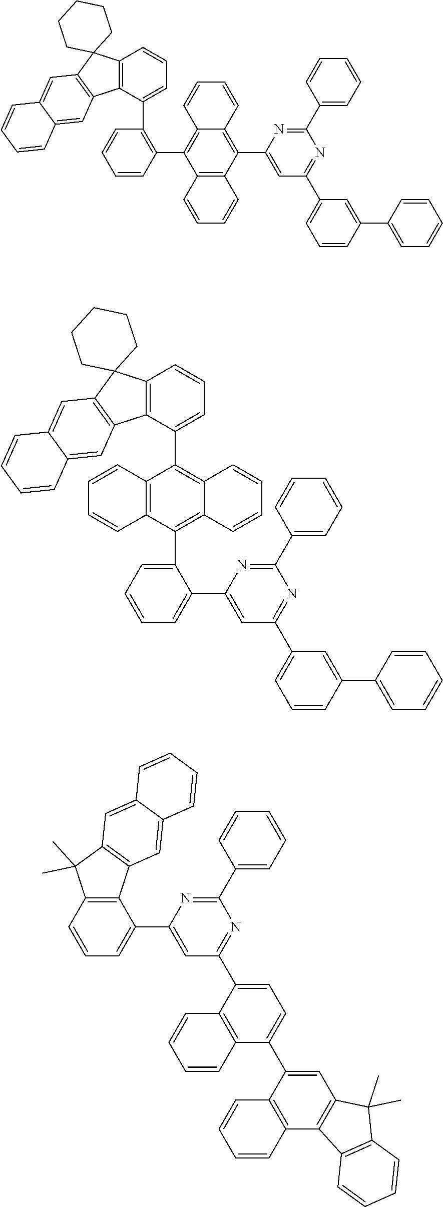

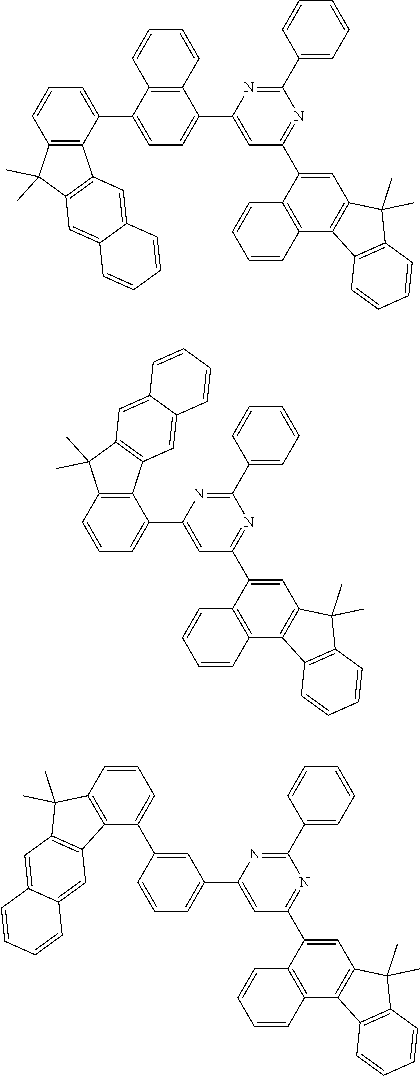

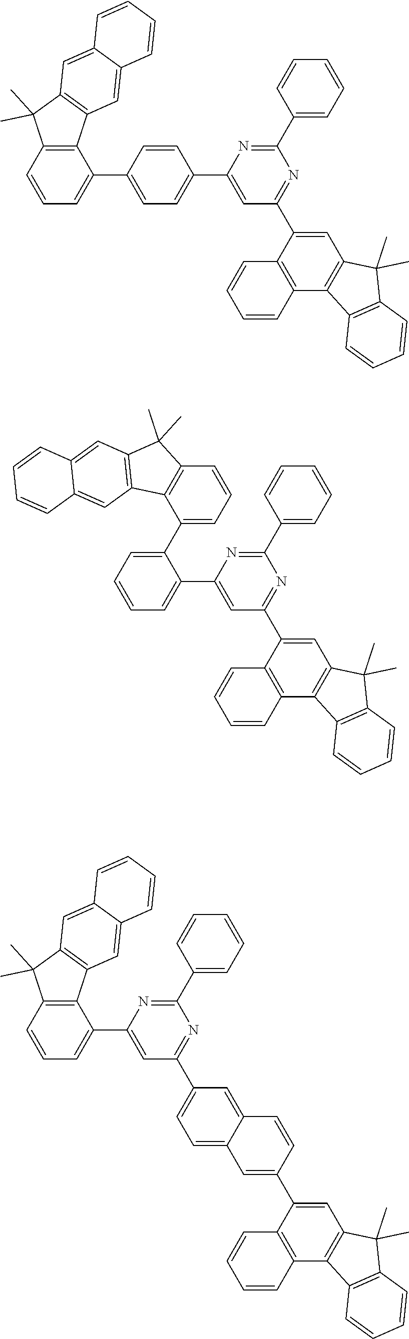

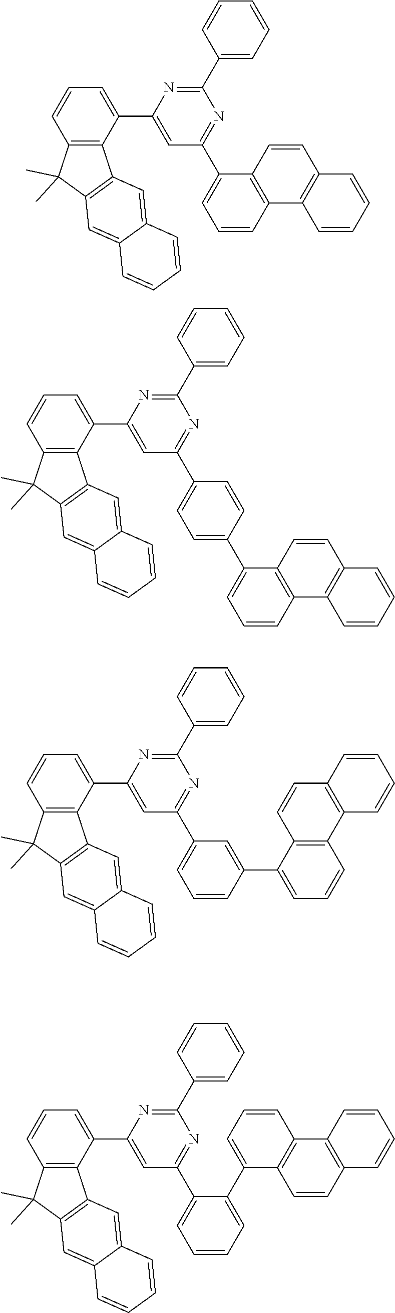

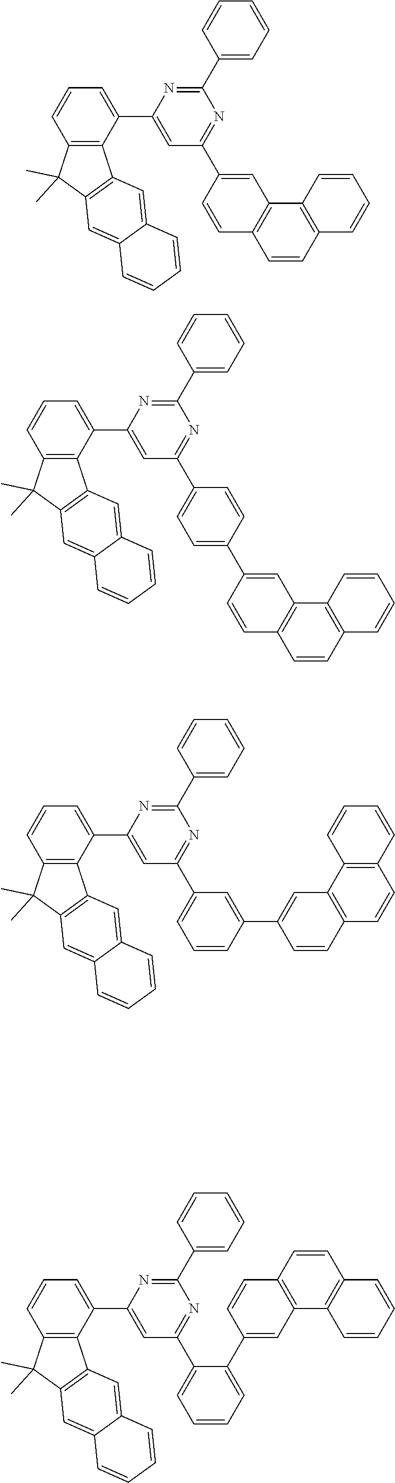

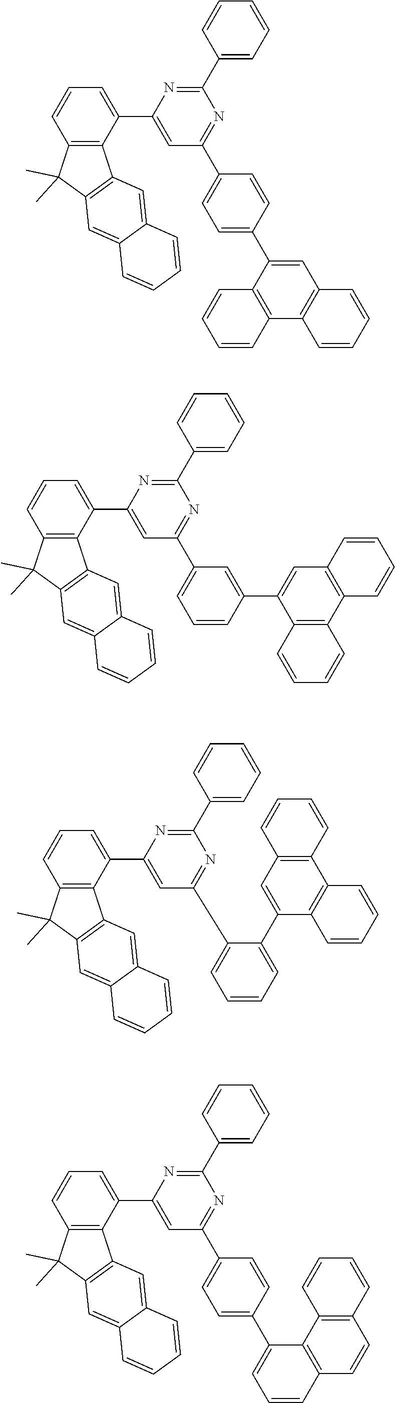 [SMILES: CC1(C)c2cc3ccccc3cc2-c2c(-c3cc(-c4ccc(-c5cc6ccccc6c6ccccc56)cc4)nc(-c4ccccc4)n3)cccc21 ; CC1(C)c2cc3ccccc3cc2-c2c(-c3cc(-c4cccc(-c5cc6ccccc6c6ccccc56)c4)nc(-c4ccccc4)n3)cccc21 ; CC1(C)c2cc3ccccc3cc2-c2c(-c3cc(-c4ccccc4-c4cc5ccccc5c5ccccc45)nc(-c4ccccc4)n3)cccc21 ; CC1(C)c2cc3ccccc3cc2-c2c(-c3cc(-c4ccc(-c5cccc6ccc7ccccc7c56)cc4)nc(-c4ccccc4)n3)cccc21]

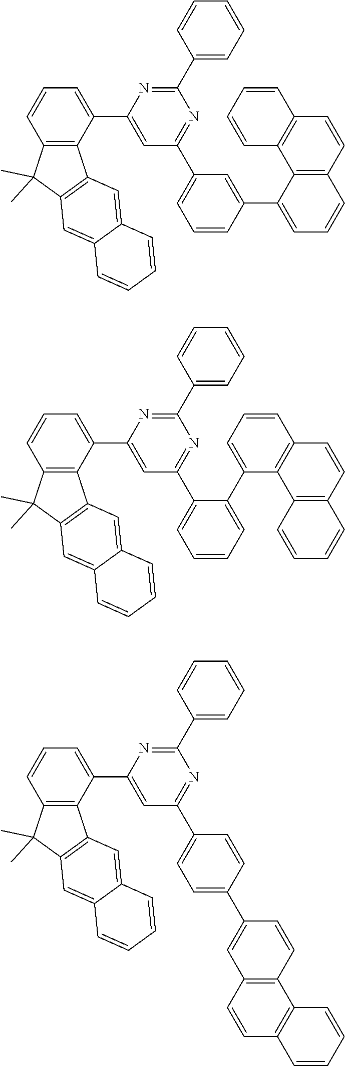 [SMILES: CC1(C)c2cc3ccccc3cc2-c2c(-c3cc(-c4cccc(-c5cccc6ccc7ccccc7c56)c4)nc(-c4ccccc4)n3)cccc21 ; CC1(C)c2cc3ccccc3cc2-c2c(-c3cc(-c4ccccc4-c4cccc5ccc6ccccc6c45)nc(-c4ccccc4)n3)cccc21 ; CC1(C)c2cc3ccccc3cc2-c2c(-c3cc(-c4ccc(-c5ccc6c(ccc7ccccc76)c5)cc4)nc(-c4ccccc4)n3)cccc21]

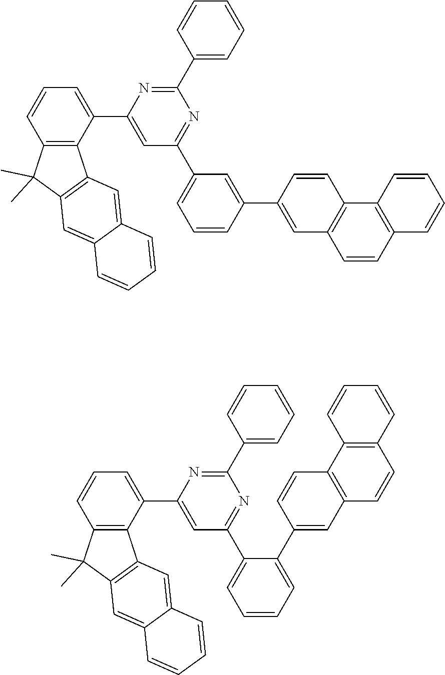 [SMILES: CC1(C)c2cc3ccccc3cc2-c2c(-c3cc(-c4cccc(-c5ccc6c(ccc7ccccc76)c5)c4)nc(-c4ccccc4)n3)cccc21 ; CC1(C)c2cc3ccccc3cc2-c2c(-c3cc(-c4ccccc4-c4ccc5c(ccc6ccccc65)c4)nc(-c4ccccc4)n3)cccc21]

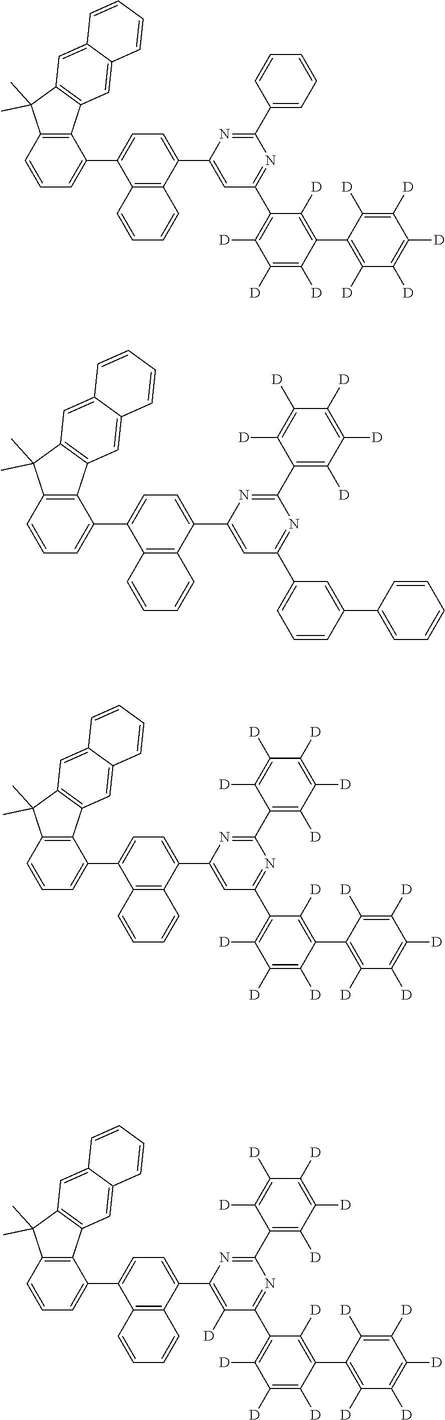

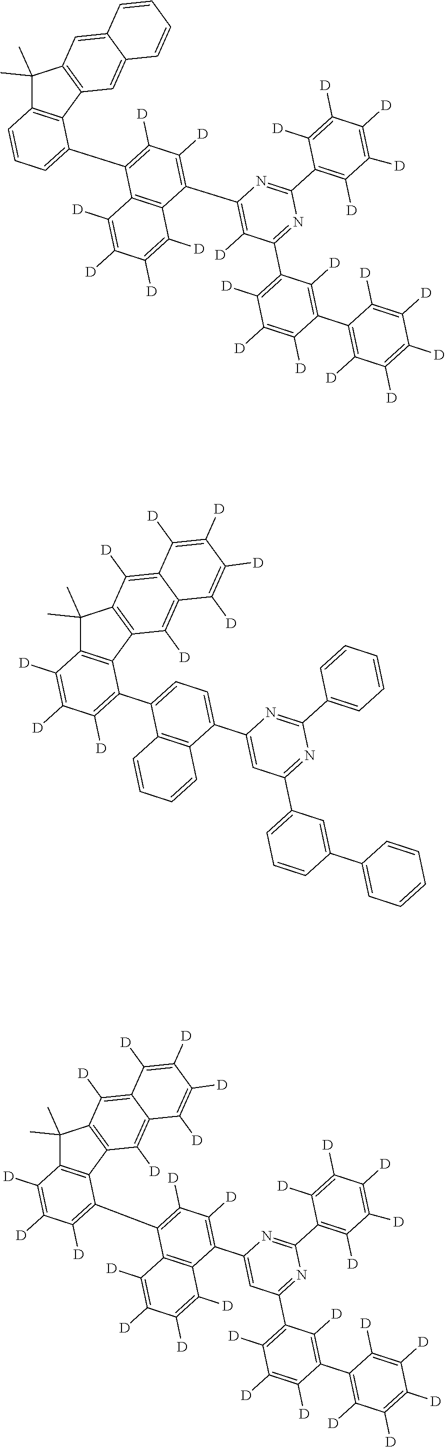

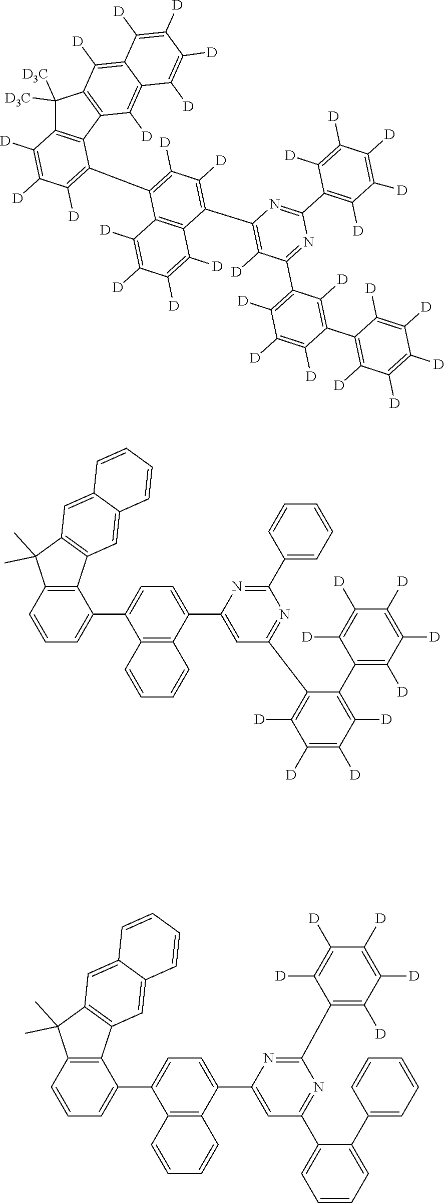

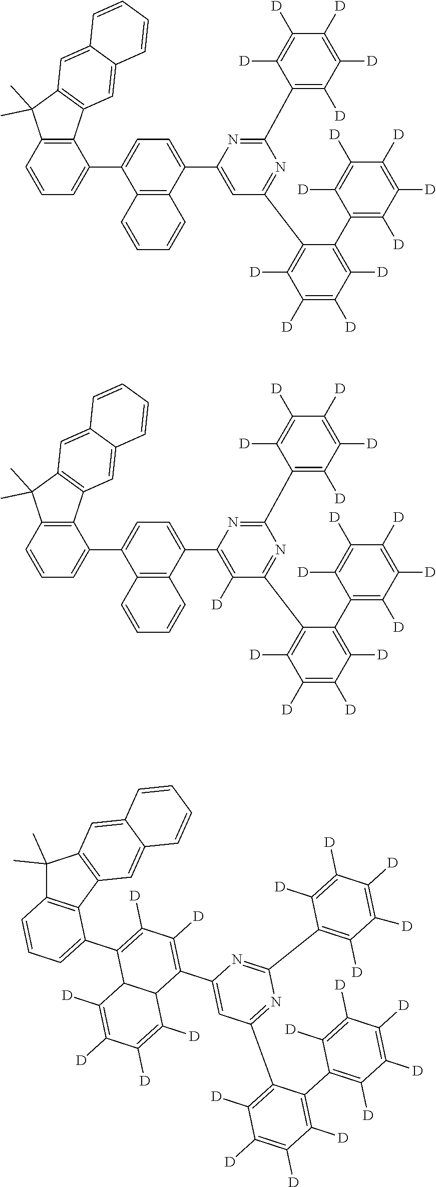

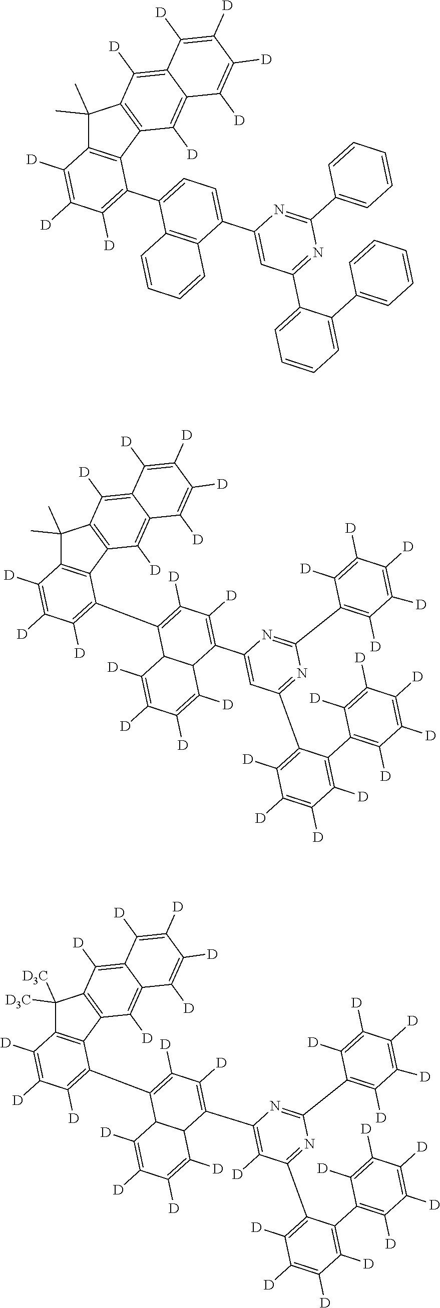

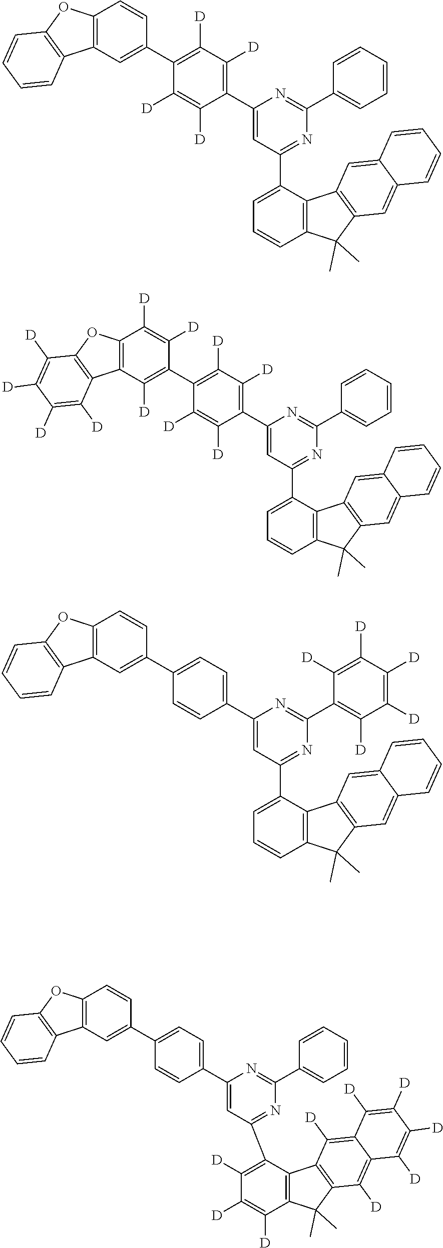

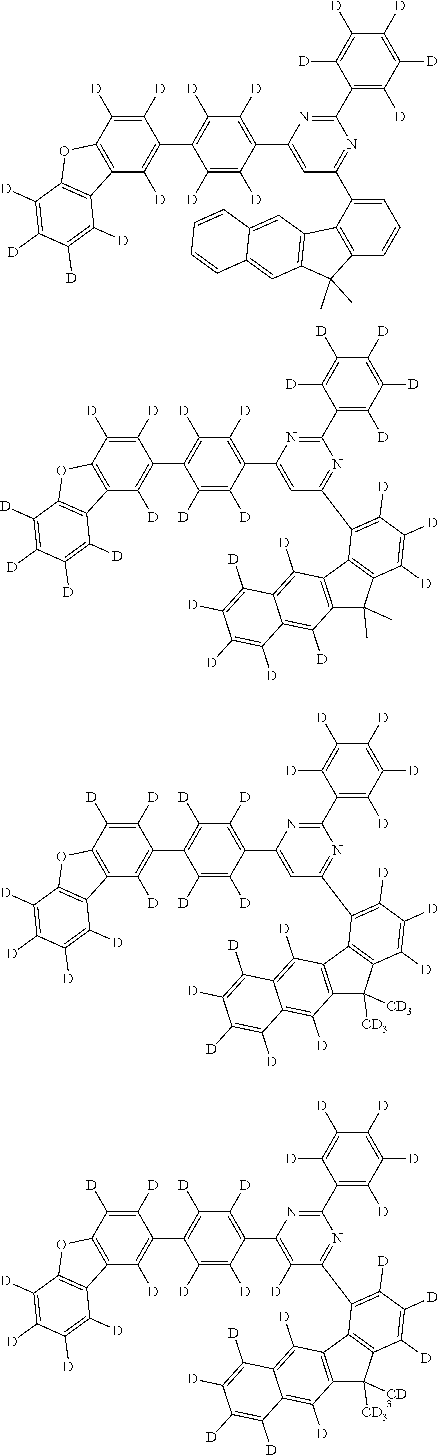

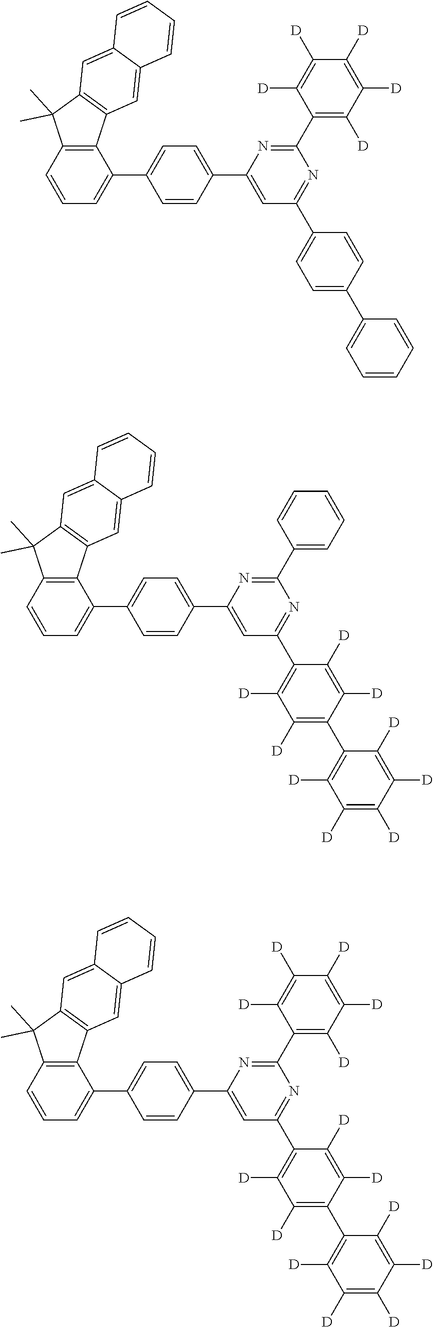 [SMILES: [2H]c1c([2H])c([2H])c(-c2nc(-c3ccc(-c4ccccc4)cc3)cc(-c3ccc(-c4cccc5c4-c4cc6ccccc6cc4C5(C)C)cc3)n2)c([2H])c1[2H] ; [2H]c1c([2H])c([2H])c(-c2c([2H])c([2H])c(-c3cc(-c4ccc(-c5cccc6c5-c5cc7ccccc7cc5C6(C)C)cc4)nc(-c4ccccc4)n3)c([2H])c2[2H])c([2H])c1[2H] ; [2H]c1c([2H])c([2H])c(-c2nc(-c3ccc(-c4cccc5c4-c4cc6ccccc6cc4C5(C)C)cc3)cc(-c3c([2H])c([2H])c(-c4c([2H])c([2H])c([2H])c([2H])c4[2H])c([2H])c3[2H])n2)c([2H])c1[2H]]

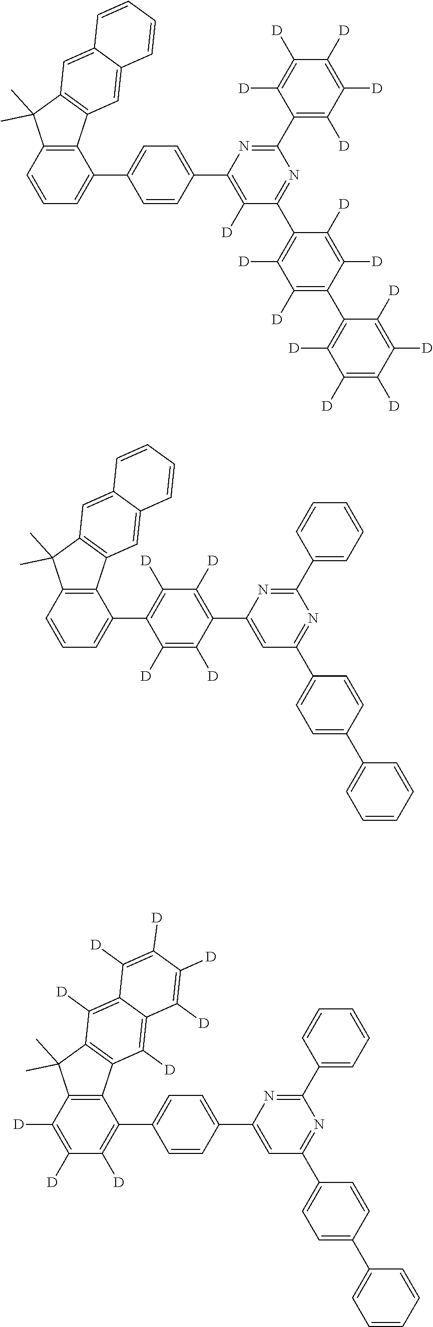 [SMILES: [2H]c1c(-c2ccc(-c3cccc4c3-c3cc5ccccc5cc3C4(C)C)cc2)nc(-c2c([2H])c([2H])c([2H])c([2H])c2[2H])nc1-c1c([2H])c([2H])c(-c2c([2H])c([2H])c([2H])c([2H])c2[2H])c([2H])c1[2H] ; [2H]c1c([2H])c(-c2cccc3c2-c2cc4ccccc4cc2C3(C)C)c([2H])c([2H])c1-c1cc(-c2ccc(-c3ccccc3)cc2)nc(-c2ccccc2)n1 ; [2H]c1c([2H])c(-c2ccc(-c3cc(-c4ccc(-c5ccccc5)cc4)nc(-c4ccccc4)n3)cc2)c2c(c1[2H])C(C)(C)c1c-2c([2H])c2c([2H])c([2H])c([2H])c([2H])c2c1[2H]]

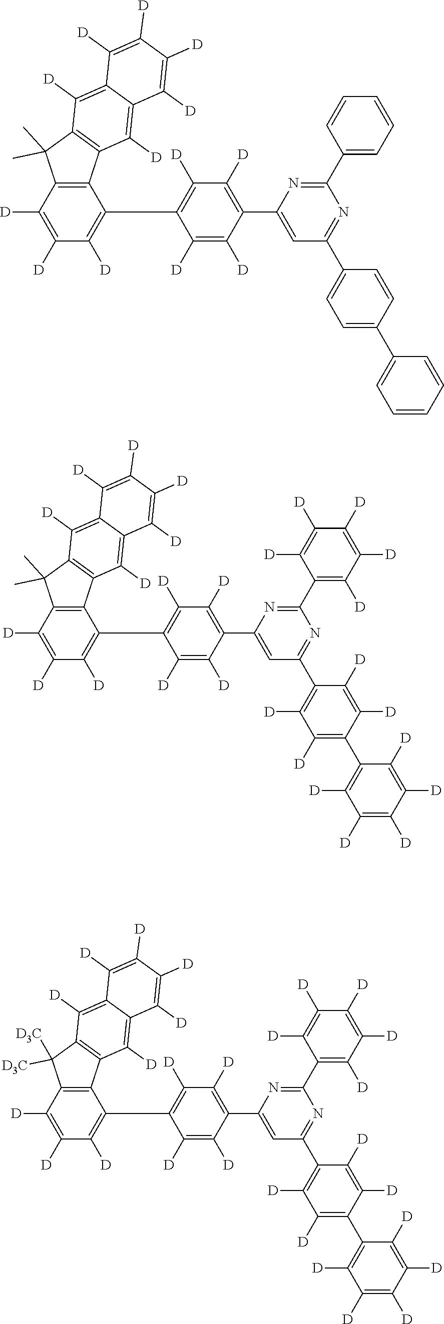

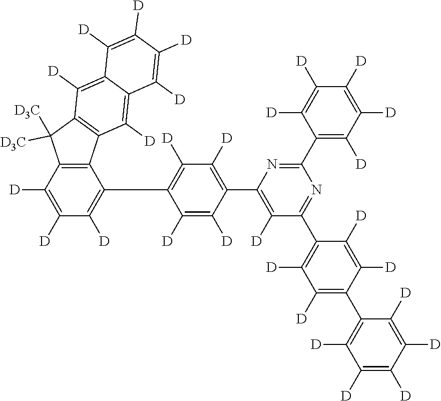 [SMILES: [2H]c1c(-c2c([2H])c([2H])c(-c3c([2H])c([2H])c([2H])c([2H])c3[2H])c([2H])c2[2H])nc(-c2c([2H])c([2H])c([2H])c([2H])c2[2H])nc1-c1c([2H])c([2H])c(-c2c([2H])c([2H])c([2H])c3c2-c2c(c([2H])c4c([2H])c([2H])c([2H])c([2H])c4c2[2H])C3(C([2H])([2H])[2H])C([2H])([2H])[2H])c([2H])c1[2H]]

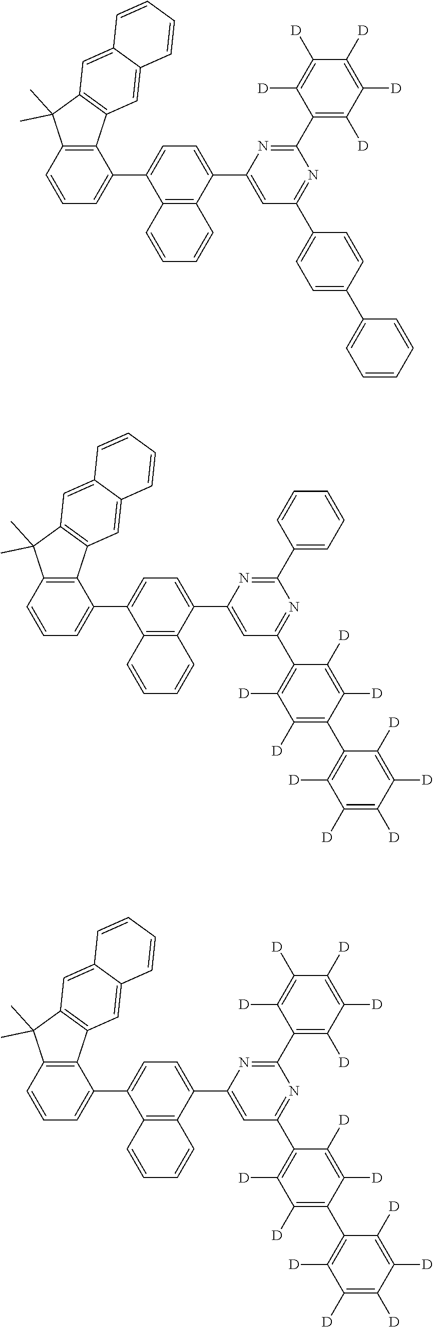 [SMILES: [2H]c1c([2H])c([2H])c(-c2nc(-c3ccc(-c4ccccc4)cc3)cc(-c3ccc(-c4cccc5c4-c4cc6ccccc6cc4C5(C)C)c4ccccc34)n2)c([2H])c1[2H] ; [2H]c1c([2H])c([2H])c(-c2c([2H])c([2H])c(-c3cc(-c4ccc(-c5cccc6c5-c5cc7ccccc7cc5C6(C)C)c5ccccc45)nc(-c4ccccc4)n3)c([2H])c2[2H])c([2H])c1[2H] ; [2H]c1c([2H])c([2H])c(-c2nc(-c3c([2H])c([2H])c(-c4c([2H])c([2H])c([2H])c([2H])c4[2H])c([2H])c3[2H])cc(-c3ccc(-c4cccc5c4-c4cc6ccccc6cc4C5(C)C)c4ccccc34)n2)c([2H])c1[2H]]

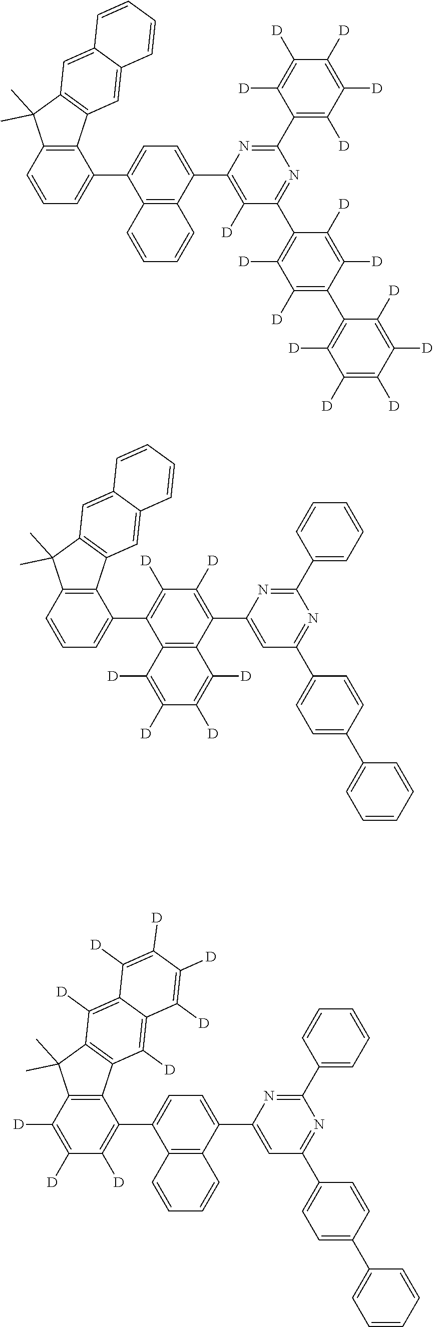 [SMILES: [2H]c1c(-c2c([2H])c([2H])c(-c3c([2H])c([2H])c([2H])c([2H])c3[2H])c([2H])c2[2H])nc(-c2c([2H])c([2H])c([2H])c([2H])c2[2H])nc1-c1ccc(-c2cccc3c2-c2cc4ccccc4cc2C3(C)C)c2ccccc12 ; [2H]c1c([2H])c([2H])c2c(-c3cccc4c3-c3cc5ccccc5cc3C4(C)C)c([2H])c([2H])c(-c3cc(-c4ccc(-c5ccccc5)cc4)nc(-c4ccccc4)n3)c2c1[2H] ; [2H]c1c([2H])c(-c2ccc(-c3cc(-c4ccc(-c5ccccc5)cc4)nc(-c4ccccc4)n3)c3ccccc23)c2c(c1[2H])C(C)(C)c1c-2c([2H])c2c([2H])c([2H])c([2H])c([2H])c2c1[2H]]

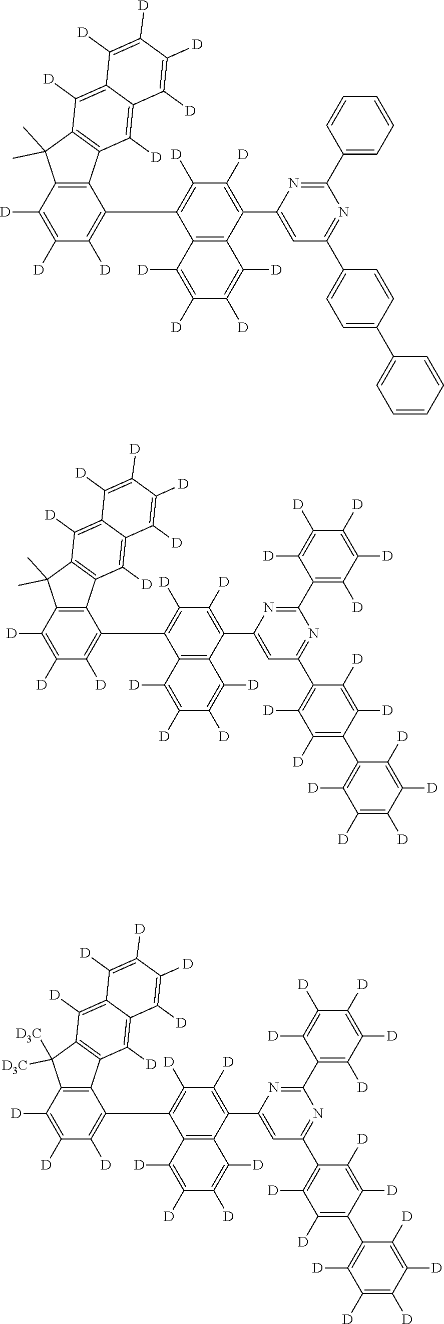

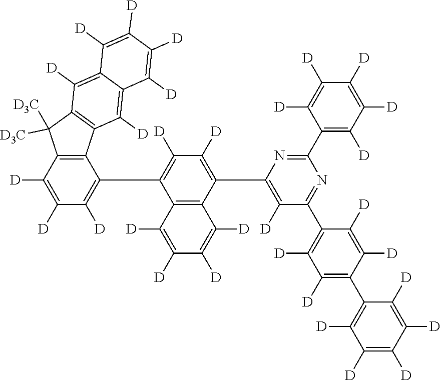 [SMILES: [2H]c1c(-c2c([2H])c([2H])c(-c3c([2H])c([2H])c([2H])c([2H])c3[2H])c([2H])c2[2H])nc(-c2c([2H])c([2H])c([2H])c([2H])c2[2H])nc1-c1c([2H])c([2H])c(-c2c([2H])c([2H])c([2H])c3c2-c2c(c([2H])c4c([2H])c([2H])c([2H])c([2H])c4c2[2H])C3(C([2H])([2H])[2H])C([2H])([2H])[2H])c2c([2H])c([2H])c([2H])c([2H])c12]

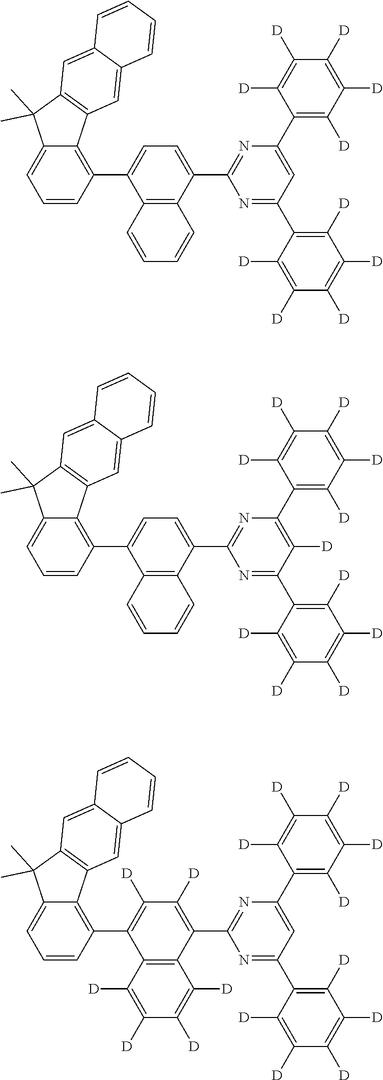 [SMILES: [2H]c1c([2H])c([2H])c(-c2cc(-c3c([2H])c([2H])c([2H])c([2H])c3[2H])nc(-c3ccc(-c4cccc5c4-c4cc6ccccc6cc4C5(C)C)c4ccccc34)n2)c([2H])c1[2H] ; [2H]c1c(-c2c([2H])c([2H])c([2H])c([2H])c2[2H])nc(-c2ccc(-c3cccc4c3-c3cc5ccccc5cc3C4(C)C)c3ccccc23)nc1-c1c([2H])c([2H])c([2H])c([2H])c1[2H] ; [2H]c1c([2H])c([2H])c(-c2cc(-c3c([2H])c([2H])c([2H])c([2H])c3[2H])nc(-c3c([2H])c([2H])c(-c4cccc5c4-c4cc6ccccc6cc4C5(C)C)c4c([2H])c([2H])c([2H])c([2H])c34)n2)c([2H])c1[2H]]

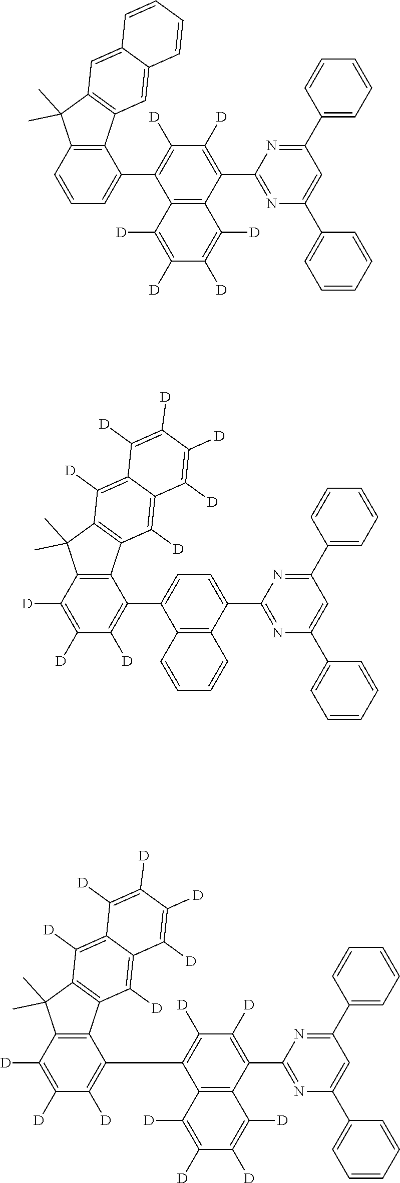 [SMILES: [2H]c1c([2H])c([2H])c2c(-c3cccc4c3-c3cc5ccccc5cc3C4(C)C)c([2H])c([2H])c(-c3nc(-c4ccccc4)cc(-c4ccccc4)n3)c2c1[2H] ; [2H]c1c([2H])c(-c2ccc(-c3nc(-c4ccccc4)cc(-c4ccccc4)n3)c3ccccc23)c2c(c1[2H])C(C)(C)c1c-2c([2H])c2c([2H])c([2H])c([2H])c([2H])c2c1[2H] ; [2H]c1c([2H])c(-c2c([2H])c([2H])c(-c3nc(-c4ccccc4)cc(-c4ccccc4)n3)c3c([2H])c([2H])c([2H])c([2H])c23)c2c(c1[2H])C(C)(C)c1c-2c([2H])c2c([2H])c([2H])c([2H])c([2H])c2c1[2H]]

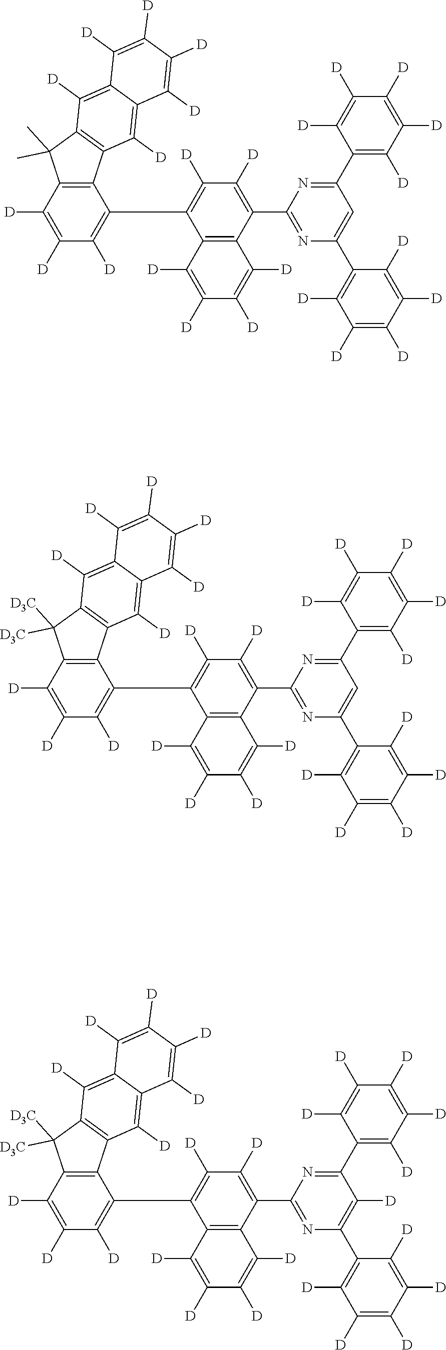

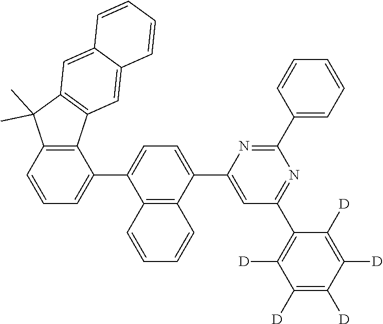 [SMILES: [2H]c1c([2H])c([2H])c(-c2cc(-c3ccc(-c4cccc5c4-c4cc6ccccc6cc4C5(C)C)c4ccccc34)nc(-c3ccccc3)n2)c([2H])c1[2H]]

### [Material for Organic Electroluminescence Device]

The compound according to one aspect of the invention is useful as a material for an organic EL device, and for example, is useful as a material used for electron-transporting region of an organic EL device.

### [Organic EL Device]

An organic EL device according to one aspect of the invention is described.

An organic EL device according to one aspect of the invention includes a cathode, an anode, and one or two or more organic layers arranged between the cathode and anode, wherein at least one of the organic layers includes the compound (represented by the formula (1)) according to one aspect of the invention.

In one embodiment, an organic EL device according to one aspect of the invention includes an anode, an emitting layer, an electron-transporting region, and a cathode in this order, and the electron-transporting region includes the compound (represented by the formula (1)) according to one aspect of the invention.

In one embodiment, the electron-transporting region includes a first layer (also referred to as a “first electron-transporting layer” or “hole barrier layer”) and a second layer (also referred to as a “second electron-transporting layer”) in this order from the emitting layer side, and the first layer includes the compound represented by the formula (1). The above-described second layer, in this case, an electron-transporting layer configuration described later can be applied.

As the typical device configuration of organic EL device, structures in which the following structures are stacked on a substrate are exemplified:
(1) an anode/an emitting layer/an electron-transporting region/a cathode
(2) an anode/a hole-transporting region/an emitting layer/an electron-transporting region/a cathode
“/” indicates that the layers are stacked directly adjacent to each other.

The electron-transporting region is generally composed of one or more layers selected from an electron-injecting layer and an electron-transporting layer. The hole-transporting region is generally composed of one or more layers selected from a hole-injecting layer and a hole-transporting layer.

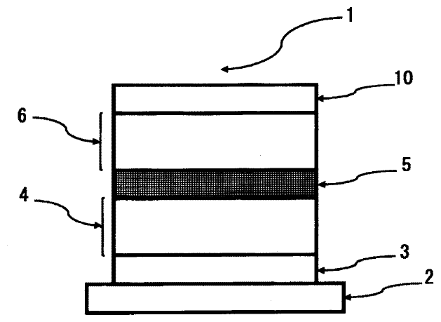

The schematic configuration of the organic EL device of one aspect of the invention will be described referring to FIG. 1.

An organic EL device 1 according to one aspect of the invention comprises a substrate 2, an anode 3, an emitting layer 5, a cathode 10, a hole-transporting region 4 between the anode 3 and the emitting layer 5, and an electron-transporting region 6 between the emitting layer 5 and the cathode 10.

Members which can be used in the organic EL device according to one aspect of the invention, materials for forming each layer, other than the above-mentioned compounds, and the like, are described.

### (Substrate)

The substrate is used as a support of an emitting device. As the substrate, glass, quartz, plastic or the like can be used, for example. Further, a flexible substrate may be used. The “flexible substrate” means a bendable (flexible) substrate, and specific examples thereof include a plastic substrate formed of polycarbonate, polyvinyl chloride, or the like.

### (Anode)

For the anode formed on the substrate, metals, alloys, electrically conductive compounds, mixtures thereof, and the like, which have a large work function (specifically 4.0 eV or more) are preferably used. Specific examples thereof include indium oxide-tin oxide (ITO:Indium Tin Oxide), indium oxide-tin oxide containing silicon or silicon oxide, indium oxide-zinc oxide, tungsten oxide, indium oxide containing zinc oxide, and graphene. In addition thereto, specific examples thereof include gold (Au), platinum (Pt), a nitride of a metallic material (for example, titanium nitride), and the like.

### (Hole-Injecting Layer)

The hole-injecting layer is a layer containing a substance having a high hole-injecting property. As the substance having high hole-injecting property, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, a polymer compound (oligomers, dendrimers, polymers, or the like) and the like can be used.

### (Hole-Transporting Layer)

The hole-transporting layer is a layer containing a substance having a high hole-transporting property. For the hole-transporting layer, an aromatic amine compound, a carbazole derivative, an anthracene derivative, and the like can be used. A polymer compound such as poly(N-vinylcarbazole) (abbreviation: PVK) and poly(4-vinyltriphenylamine) (abbreviation: PVTPA) can also be used. Provided that a substance other than the above-described substances may be used as long as the substance has a higher hole-transporting property than an electron-transporting property. The layer containing the substance having high hole-transporting property may be not only a single layer, but also layers in which two or more layers formed of the above-described substances are stacked.

### (Guest (Dopant) Material of Emitting Layer)

The emitting layer is a layer containing a substance having a high emitting property, and various materials can be used for the emitting layer. For example, as the substance having a high emitting property, a fluorescent compound which emits fluorescence or a phosphorescent compound which emits phosphorescence can be used. The fluorescent compound is a compound which can emit from a singlet excited state, and the phosphorescent compound is a compound which can emit from a triplet excited state.

As a blue fluorescent emitting material which can be used for the emitting layer, pyrene derivatives, styrylamine derivatives, chrysene derivatives, fluoranthene derivatives, fluorene derivatives, diamine derivatives, triarylamine derivatives, and the like can be used. As a green fluorescent emitting material which can be used for the emitting layer, aromatic amine derivatives and the like can be used. As a red fluorescent emitting material which can be used for the emitting layer, tetracene derivatives, diamine derivatives and the like can be used.

As a blue phosphorescent emitting material which can be used for the emitting layer, metal complexes such as iridium complexes, osmium complexes, platinum complexes and the like are used. As a green phosphorescent emitting material which can be used for the emitting layer, iridium complexes and the like are used. As a red phosphorescent emitting material which can be used for the emitting layer, metal complexes such as iridium complexes, platinum complexes, terbium complexes, europium complexes and the like can be used.

### (Host Material for Emitting Layer)

The emitting layer may have a constitution in which the substance having a high emitting property (guest material) is dispersed in another substance (host material). As such another substance for dispersing the substance having a high emitting property, a variety of substances can be used, and it is preferable to use such another substance having a higher lowest unoccupied orbital level (LUMO level) and a lower highest occupied orbital level (HOMO level) than the substance having a high emitting property.

As such another substance for dispersing the substance having a high emitting property (host material), 1) metal complexes such as aluminum complexes, beryllium complexes, zinc complexes, or the like; 2) heterocyclic compounds such as oxadiazole derivatives, benzimidazole derivatives, phenanthroline derivatives, or the like; 3) fused aromatic compounds such as carbazole derivatives, anthracene derivatives, phenanthrene derivatives, pyrene derivatives, chrysene derivatives, or the like; and 4) aromatic amine compound such as triarylamine derivatives, fused polycyclic aromatic amine derivatives, or the like are used.

### (Electron-Transporting Layer)

The electron-transporting layer is a layer containing a substance having a high electron-transporting property. For the electron-transporting layer, 1) metal complexes such as aluminum complexes, beryllium complexes, zinc complexes, or the like; 2) heteroaromatic compounds such as imidazole derivatives, benzimidazole derivatives, azine derivatives, carbazole derivatives, phenanthroline derivatives, or the like; and 3) polymer compounds can be used.

In one embodiment of the invention, the electron-transporting layer may or may not include other materials described above in addition to the compound (represented by the formula (1)) according to one aspect of the invention.

### (Electron-Injecting Layer)

The electron-injecting layer is a layer containing a substance having a high electron-injecting property. For the electron-injecting layer, lithium (Li), ytterbium (Yb), lithium fluoride (LiF), cesium fluoride (CsF), calcium fluoride (CaF₂), metal complexes such as 8-hydroxyquinolinolato-lithium (Liq), alkaline metal, alkaline earth metal, or compound thereof such as lithium oxide (LiOₓ) can be used.

### (Cathode)

For the cathode, metals, alloys, electrically conductive compounds, mixtures thereof, and the like, which have a small work function (specifically, 3.8 eV or lower) are preferably used. Specific examples of such a cathode material include elements each belonging to Group 1 or Group 2 of the Periodic Table of the Elements, i.e., alkali metals such as lithium (Li) and cesium (Cs), alkaline earth metals such as magnesium (Mg), calcium (Ca) and strontium (Sr), and alloys containing these metals (e.g., MgAg and AlLi); rare earth metals such as europium (Eu) and ytterbium (Yb), and alloys containing these metals.

The cathode is generally formed by a vacuum deposition method or a sputtering method. Further, in the case of using a silver paste or the like, a coating method, an inkjet method, or the like can be employed.

In the case where the electron-injecting layer is provided, a cathode can be formed using various electrically conductive materials such as aluminum, silver, ITO, graphene, indium oxide-tin oxide containing silicon or silicon oxide, regardless of the work function value.

In the organic EL device according to an aspect of the invention, the thickness of each layer is not particularly limited, but is generally preferable that the thickness be generally in the range of several nm to 1 μm in order to suppress defects such as pinholes, to suppress applied voltages to be low, and to increase luminous efficiency.

In the organic EL device according to one aspect of the invention, the method for forming the each layer is not particularly limited. A conventionally-known method for forming each layer, such as a vacuum deposition method, a spin coating method or the like, can be used. Each layer such as the emitting layer can be formed by a known method such as a vacuum deposition method, a molecular beam deposition method (MBE method), or an application method such as a dipping method, a spin coating method, a casting method, a bar coating method, or a roll coating method, in which a solution prepared by dissolving the material in a solvent is used.

### [Electronic Apparatus]

The electronic apparatus according to an aspect of the invention is equipped with the organic EL device according to an aspect of the invention.

Specific examples of the electronic apparatus include display components such as an organic EL panel module, and the like; display devices such as a television, a cellular phone, a personal computer, and the like; and emitting devices such as a light, a vehicular lamp, and the like.

### EXAMPLES

### <Compound>

Compounds represented by the formula (1) used in the fabrication of organic EL devices of Examples are shown below.

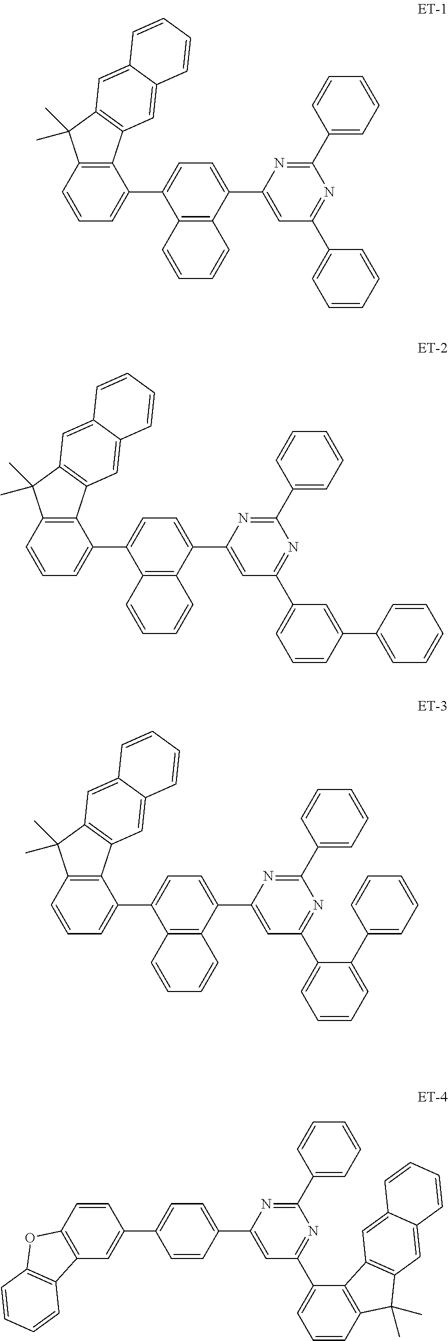 [SMILES: CC1(C)c2cc3ccccc3cc2-c2c(-c3ccc(-c4cc(-c5ccccc5)nc(-c5ccccc5)n4)c4ccccc34)cccc21 ; CC1(C)c2cc3ccccc3cc2-c2c(-c3ccc(-c4cc(-c5cccc(-c6ccccc6)c5)nc(-c5ccccc5)n4)c4ccccc34)cccc21 ; CC1(C)c2cc3ccccc3cc2-c2c(-c3ccc(-c4cc(-c5ccccc5-c5ccccc5)nc(-c5ccccc5)n4)c4ccccc34)cccc21 ; CC1(C)c2cc3ccccc3cc2-c2c(-c3cc(-c4ccc(-c5ccc6oc7ccccc7c6c5)cc4)nc(-c4ccccc4)n3)cccc21]

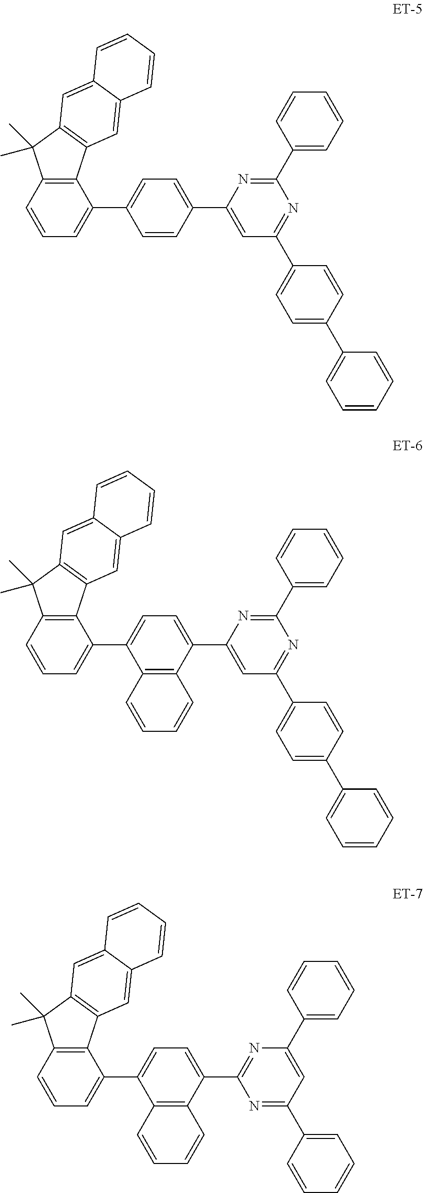 [SMILES: CC1(C)c2cc3ccccc3cc2-c2c(-c3ccc(-c4cc(-c5ccc(-c6ccccc6)cc5)nc(-c5ccccc5)n4)cc3)cccc21 ; CC1(C)c2cc3ccccc3cc2-c2c(-c3ccc(-c4cc(-c5ccc(-c6ccccc6)cc5)nc(-c5ccccc5)n4)c4ccccc34)cccc21 ; CC1(C)c2cc3ccccc3cc2-c2c(-c3ccc(-c4nc(-c5ccccc5)cc(-c5ccccc5)n4)c4ccccc34)cccc21]

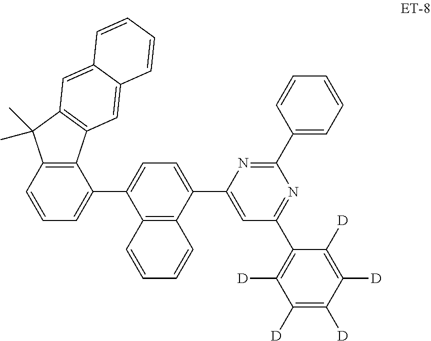 [SMILES: [2H]c1c([2H])c([2H])c(-c2cc(-c3ccc(-c4cccc5c4-c4cc6ccccc6cc4C5(C)C)c4ccccc34)nc(-c3ccccc3)n2)c([2H])c1[2H]]

Compounds used in the fabrication of organic EL devices of the Comparative Examples are shown below.

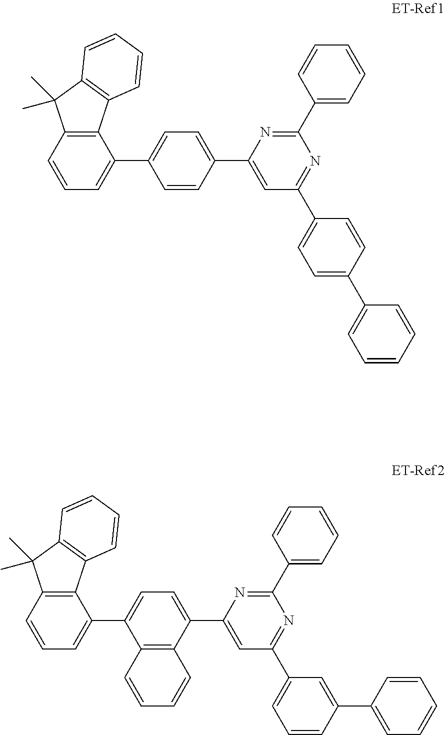 [SMILES: CC1(C)c2ccccc2-c2c(-c3ccc(-c4cc(-c5ccc(-c6ccccc6)cc5)nc(-c5ccccc5)n4)cc3)cccc21 ; CC1(C)c2ccccc2-c2c(-c3ccc(-c4cc(-c5cccc(-c6ccccc6)c5)nc(-c5ccccc5)n4)c4ccccc34)cccc21]

Other compounds used in the fabrication of organic EL devices of Examples and Comparative Examples are shown below.

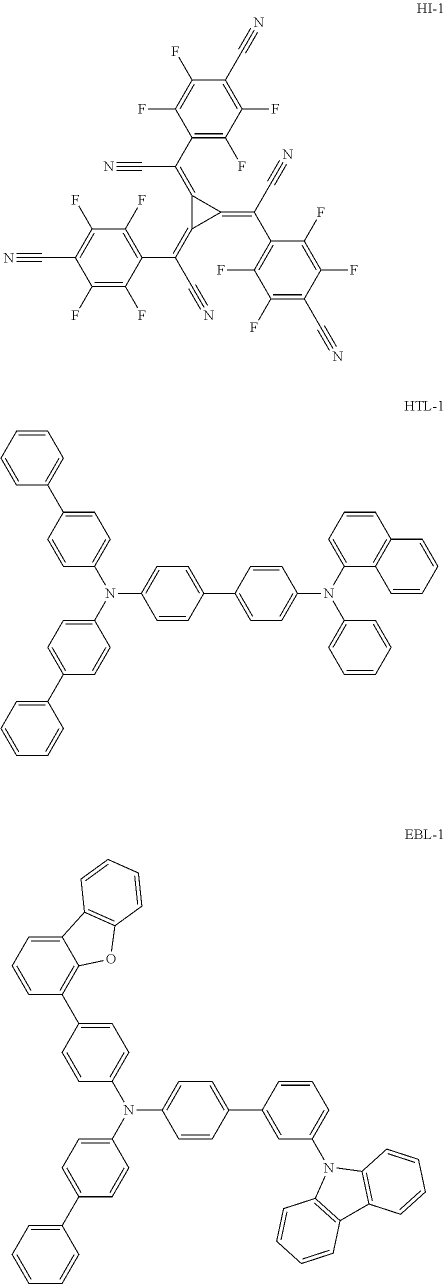 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4ccccc4)cc3)c3ccc(-c4ccc(N(c5ccccc5)c5cccc6ccccc56)cc4)cc3)cc2)cc1 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

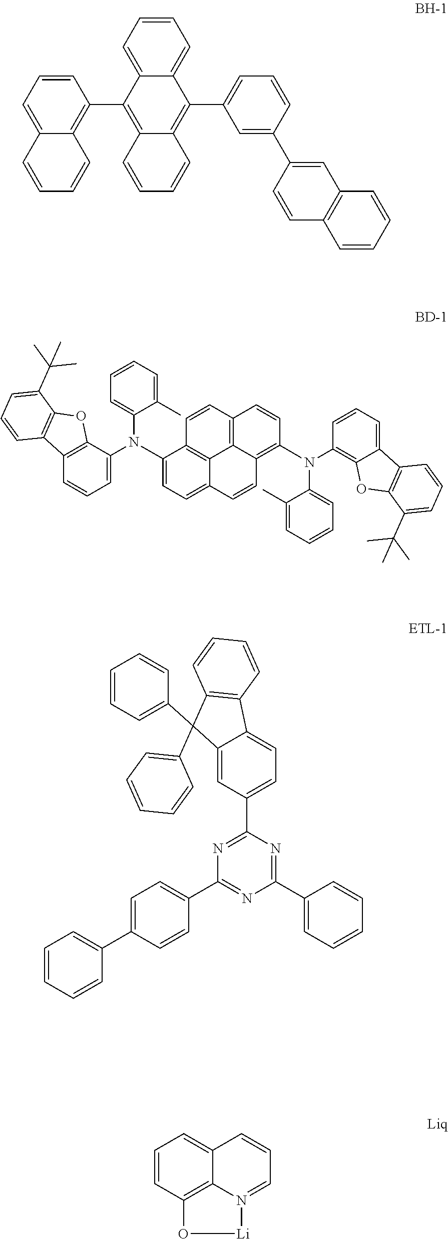

### Example 1

### <Fabrication of Organic EL Device>

An organic EL device was fabricated as follows.

A 25 mm×75 mm×1.1 mm-thick glass substrate with an ITO transparent electrode (anode) (manufactured by GEOMATEC) was subjected to ultrasonic cleaning in isopropyl alcohol for 5 minutes, and then subjected to UV-ozone cleaning for 30 minutes. The thickness of the ITO film was 130 nm.

The glass substrate with a transparent electrode after cleaning was mounted on a substrate holder of a vacuum vapor deposition apparatus. First, Compounds HTL-1 and HI-1 were co-deposited on the surface of the substrate of which the transparent electrode has been formed, such that the proportion of Compound HI-1 became 3% by mass to cover the transparent electrode with the compounds. Thus, a hole-injecting layer having a thickness of 10 nm was formed.

On the hole-injecting layer, Compound HTL-1 was deposited to form a first hole-transporting layer having a film thickness of 80 nm.

On the first hole-transporting layer, Compound EBL-1 was deposited to form a second hole-transporting layer having a film thickness of 5 nm.

On the second hole-transporting layer, Compound BH-1 (host material) and Compound BD-1 (dopant material) were co-deposited such that the proportion of Compound BD-1 was 4% by mass, to form an emitting layer having a film thickness of 25 nm.

On the emitting layer, Compound ET-1 was deposited to form a first electron-transporting layer (hole barrier layer) having a film thickness of 5 nm.

On the first electron-transporting layer, Compound ETL-1 and Liq were co-deposited such that the proportion of Liq was 50% by mass, to form a second electron-transporting layer having a film thickness of 20 nm.

Metal Yb was deposited on the second electron-transporting layer to form an electron-injecting layer having a film thickness of 1 nm.

Metal Al was deposited on the electron-injecting layer to form a cathode having a film thickness of 50 nm.

The device configuration of the organic EL device of Example 1 is schematically described as follows:

$$ITO ( 130 ) / HTL - 1 : HI - 1 ( 10 : 3 \% ) / HTL - 1 ( 80 ) / EBL \text{} - 1 ( 5 ) / BH - 1 : BD - 1 ( 25 : 4 \% ) / compound ET - 1 ( 5 ) / ETL \text{} - 1 : Liq ( 20 : 50 \% ) / Yb ( 1 ) / Al ( 50 )$$

Numerical values in parentheses indicate film thickness (unit: nm). In addition, the numerical values represented by percent in parentheses each indicate the proportion (% by mass) of the latter compound in the corresponding layer.

### <Evaluation of Organic EL Device>

An organic EL device fabricated was evaluated as follows. The results are shown in Table 1.

### External Quantum-Efficiency (EQE)

A voltage was applied to the organic EL device such that the current density became 10 mA/cm², and EL emission spectrum was measured by a spectral radiance meter CS-2000 (manufactured by KONICA MINOLTA). The EQE (%) was calculated from the obtained spectral radiance spectrum. In Tables 1, the EQE ratios are relative values when the value of Comparative Example 1 to be described later is taken as 100.

### Examples 2 to 6 and Comparative Examples 1 to 2

Organic EL devices were prepared and evaluated in the same manner as in Example 1, except that the compound listed in Table 1 was used instead of ET-1. The results are shown in Table 1.

TABLE 1

| | First electron- | | |
|---|---|---|---|
| | transporting layer | EQE (%) | EQE ratio (%) |
| Example1 | ET-1 | 9.7 | 113 |
| Example2 | ET-2 | 9.7 | 113 |
| Example3 | ET-5 | 9.6 | 112 |
| Example4 | ET-6 | 9.5 | 110 |
| Example5 | ET-7 | 9.9 | 115 |
| Example6 | ET-8 | 9.8 | 114 |
| Comparative | ET-Ref1 | 8.6 | 100 |
| Example1 | | | |
| Comparative | ET-Ref2 | 8.6 | 100 |
| Example2 | | | |

### Example 7

On a glass substrate with an ITO transparent electrode (anode), a hole-transporting region, an emitting layer, an electron-transporting region, and a cathode were provided to fabricate an organic EL device. The organic layer most near the emitting layer in the electron-transporting region (hole barrier layer) was formed with ET-3.

The obtained organic EL device was evaluated and found to have excellent device performance.

### Example 8

An organic EL device was fabricated and evaluated in the same manner as in Example 7 except that the organic layer most near the emitting layer in the electron-transporting region (hole barrier layer) was formed with ET-4. It was found that the device has excellent device performance.

### SYNTHESIS OF COMPOUNDS

### (Synthesis Example 1) Synthesis of ET-1

ET-1 was synthesized in accordance with the following synthetic route.

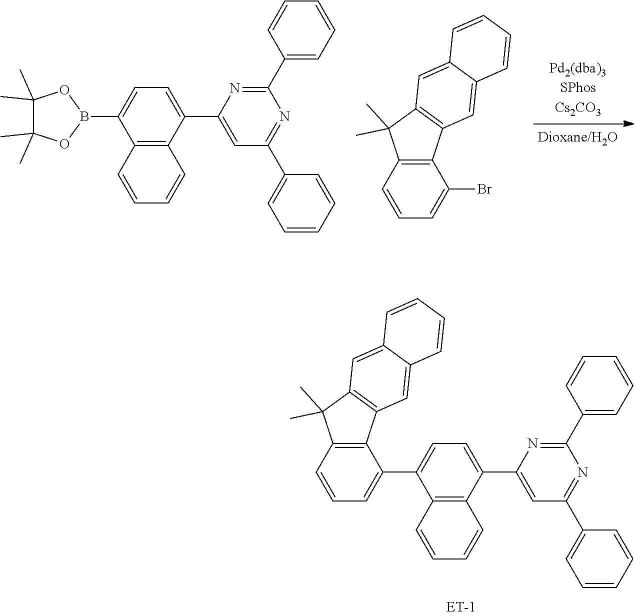 [SMILES: CC1(C)OB(c2ccc(-c3cc(-c4ccccc4)nc(-c4ccccc4)n3)c3ccccc23)OC1(C)C ; CC1(C)c2cc3ccccc3cc2-c2c(Br)cccc21 ; CC1(C)c2cc3ccccc3cc2-c2c(-c3ccc(-c4cc(-c5ccccc5)nc(-c5ccccc5)n4)c4ccccc34)cccc21]

In a flask, 2,4-Diphenyl-6-(4-(4,4,5,5-tetramethyl-1,3,2-dioxaborolan-2-yl)naphthalene-1-yl)pyrimidine (5.32 g), 4-bromo-11,11-dimethyl-11H-benzo[b]fluorene (3.55 g), Pd₂(dba)₃ (0.20 g), SPhos(0.36 g), and cesium carbonate (7.16 g) were placed, and the atmosphere in the flask was replaced with argon gas. Then, 1,4-dioxane (47 mL) and H₂O (7.8 mL) were added to the flask, and the solution was stirred with heat at 75° C. for 20 hours. After cooling the reaction solution, and distilling off the solvent, the resultant crude product was purified by silica gel chromatography, followed by washing with hexane, to obtain ET-1 as white solids (5.65 g, yield: 85%).

As a result of the mass spectrum analysis, m/e=601 with respect to the molecular weight of 600.76, so that the product was identified as the desired compound.

### (Synthesis Example 2) Synthesis of ET-2

ET-2 was synthesized in accordance with the following synthetic route.

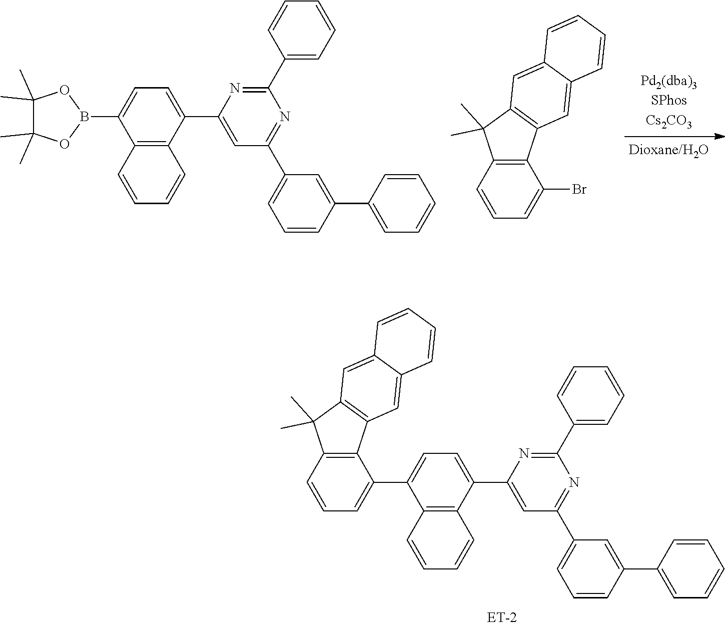

In a flask, 4-([1,1′ biphenyl]-3-yl)-2-phenyl-6-(4-(4,4,5,5-tetramethyl-1,3,2-dioxaborolan-2-yl)naphthalene-1-yl)pyrimidine (5.76 g), 4-bromo-11,11-dimethyl-11H-benzo[b]fluorene (3.32 g), Pd₂(dba)₃ (0.19 g), SPhos(0.34 g), and cesium carbonate (6.69 g) were placed, and the atmosphere in the flask was replaced with argon gas. Then, 1,4-dioxane (44 mL) and H₂O (7.3 mL) were added to the flask, and the solutions were stirred with heat at 75° C. for 20 hours. After cooling the reaction solution and distilling off the solvent, the resultant crude product was purified by silica gel chromatography and washed with hexane/acetic acid ethyl to obtain an ET-2 as white solids (6.34 g, yield: 91%).

As a result of the mass spectrum analysis, m/e=677 with respect to the molecular weight of 676.86, so that the product was identified as the desired compound.

### (Synthesis Example 3) Synthesis of ET-3

ET-3 was synthesized in accordance with the following synthetic route.

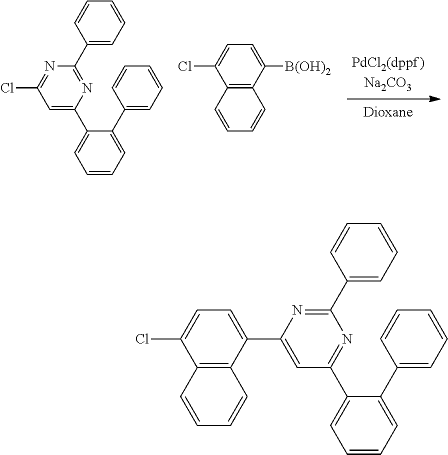 [SMILES: Clc1cc(-c2ccccc2-c2ccccc2)nc(-c2ccccc2)n1 ; OB(O)c1ccc(Cl)c2ccccc12 ; Clc1ccc(-c2cc(-c3ccccc3-c3ccccc3)nc(-c3ccccc3)n2)c2ccccc12]

In a reaction container, 4-([1,1′-biphenyl]-2-yl)-6-chloro-2-phenylpyrimidine (12.3 g), (4-chloronaphthalen-1-yl)boronic acid (7.76 g), and PdCl₂ (dppf) (1.06 g) were placed, and the atmosphere in the reaction container was replaced with argon gas. Then, dioxane (358 mL) and an aqueous sodium carbonate solution (2M, 45 mL) were added thereto, and the mixture was stirred with heat at 80° C. for 7 hours. After the solvent of the reaction solution was evaporated, followed by purification by silica gel column chromatography (developing solvent:hexane/dichloromethane) to obtain 4-([1,1′-biphenyl]-2-yl)-6-(4-chloronaphthalen-1-yl)-2-phenylpyrimidine as white solids (10.74 g, yield: 51%).

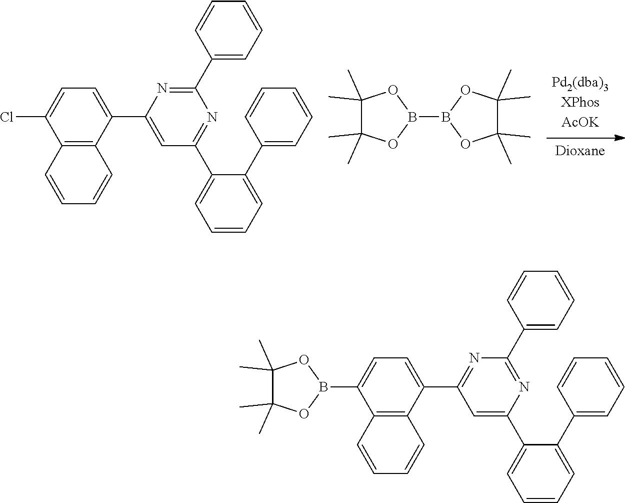

In a reaction container, 4-([1,1′-biphenyl]-2-yl)-6-(4-chloronaphthalen-1-yl)-2-phenylpyrimidine (10.74 g) and 4,4,4′,4′,5,5,5′,5′-octamethyl-2,2′-bi(1,3,2-dioxaborolane) (6.98 g), Pd₂(dba)₃ (0.42 g), XPhos(0.75 g), and potassium acetate (4.50 g) were placed, and the atmosphere in the reaction container was replaced with argon gas. Then, dioxane (76 mL) was added thereto, and the mixture was refluxed with heat for 6 hours under stirring. After distilling off the solvent of the reaction solution, followed by purification by silica gel column chromatography (developing solvent:hexane/dichloromethane) to obtain 4-([1,1′ biphenyl]-2-yl)-2-phenyl-6-(4-(4,4,5,5-tetramethyl-1,3,2-dioxaborolan-2-yl)naphthalene-1-yl)pyrimidine as white solids (6.67 g, yield: 52%).

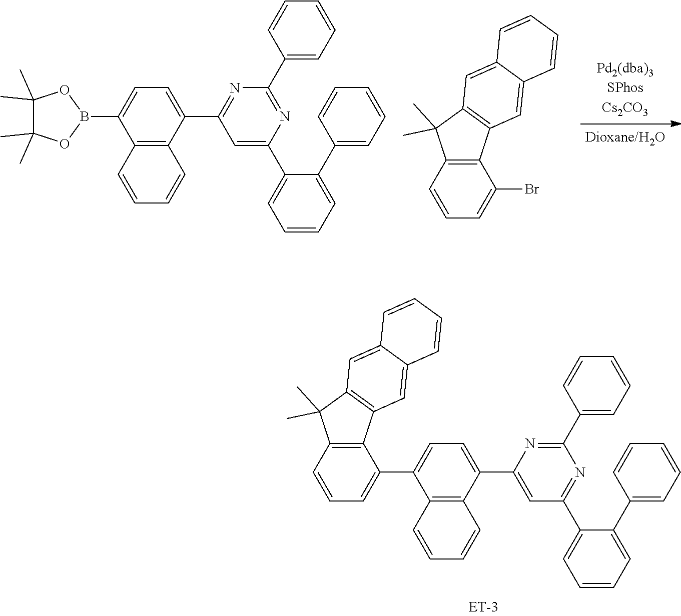

In a flask, 4-([1,1′-biphenyl]-2-yl)-2-phenyl-6-(4-(4,4,5,5-tetramethyl-1,3,2-dioxaborolan-2-yl)naphthalen-1-yl)pyrimidine (4.90 g), 4-bromo-11,11-dimethyl-11H-benzo[b]fluorene (3.11 g), Pd₂(dba)₃ (0.16 g), SPhos (0.29 g), and cesium carbonate (5.70 g) were placed, and the atmosphere in the flask was replaced with argon gas. Then, 1,4-dioxane (38 mL) and H₂O (6.2 mL) were added thereto, and the mixture was refluxed with heat for 7 hours under stirring. After cooling the reaction solution and distilling off the solvent, the resultant crude product was purified by silica gel chromatography and washed with hexane/acetic acid ethyl to obtain ET-3 as white solids (4.78 g, yield: 81%).

As a result of the mass spectrum analysis, m/e=677 with respect to the molecular weight of 676.86, so that the product was identified as the desired compound.

### (Synthesis Example 4) Synthesis of ET-4

ET-4 was synthesized in accordance with the following synthetic route.

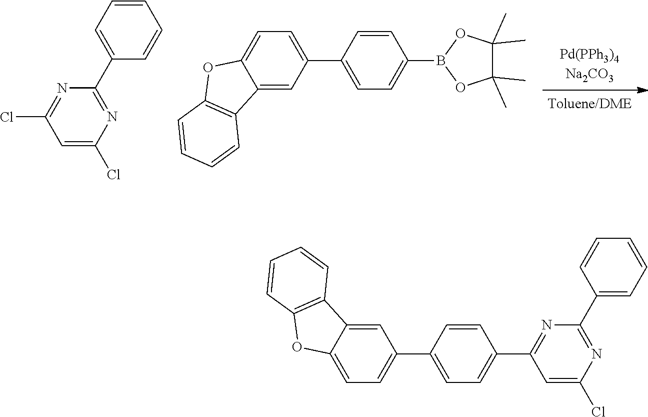 [SMILES: Clc1cc(Cl)nc(-c2ccccc2)n1 ; CC1(C)OB(c2ccc(-c3ccc4oc5ccccc5c4c3)cc2)OC1(C)C ; Clc1cc(-c2ccc(-c3ccc4oc5ccccc5c4c3)cc2)nc(-c2ccccc2)n1]

In a reaction container, 4,6-Dichloro-2-phenylpyrimidine (2.74 g), 2-(4-(dibenzo[b,d]furan-2-yl)phenyl)-4,4,5,5-tetramethyl-1,3,2-dioxaborolane (3.00 g), and Pd(PPh₃)₄ (0.38 g) were placed, and the atmosphere in the reaction container was replaced with argon gas. Then, toluene (54 mL), DME (27 mL), and an aqueous sodium carbonate solution (2M, 12 mL) were added thereto, and the mixture was heated at 80° C. for 24 hours under stirring. The solvent was distilled off from the reaction solution, followed by purification by silica gel column chromatography (developing solvent:hexane/dichloromethane) to obtain 4-chloro-6-(4-(dibenzo[b,d]furan-2-yl)phenyl)-2-phenylpyrimidine as white solids (1.25 g, yield: 36%).

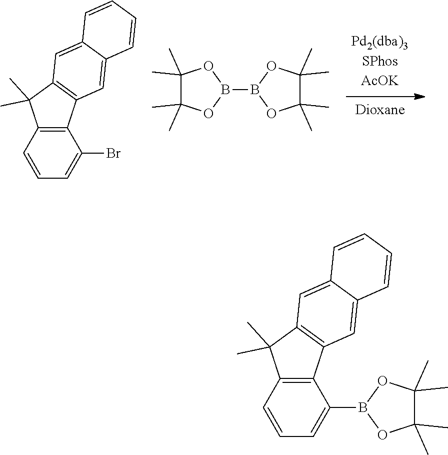

In a reaction container, 4-bromo-11,11-dimethyl-11H-benzo[b]fluorene (1.65 g) and 4,4,4′,4′,5,5,5′,5′-octamethyl-2,2′-bi(1,3,2-dioxaborolane) (1.56 g), Pd₂(dba)₃ (0.09 g), SPhos (0.17 g), and potassium acetate (1.00 g) were placed, and the atmosphere in the reaction container was replaced with argon gas. Then, dioxane (17 mL) was added thereto, and the mixture was refluxed with heat for 6 hours under stirring. The solvent was distilled off from the reaction solution, followed by purification by silica gel column chromatography (developing solvent:hexane/dichloromethane) to obtain 2-(11,11-dimethyl-11H-benzo[b]fluorene-4-yl)-4,4,5,5-tetramethyl-1,3,2-dioxaborolane as white solids (1.45 g, yield: 77%).

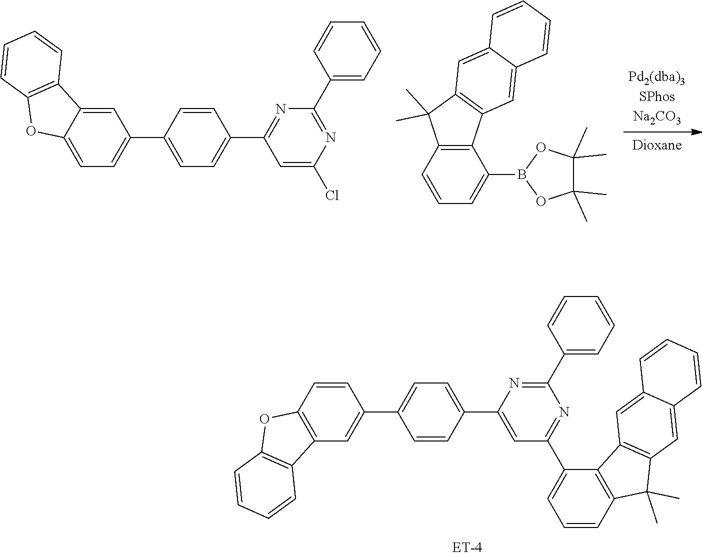

In a flask, 4-Chloro-6-(4-(dibenzo[b,d]furan-2-yl)phenyl)-2-phenylpyrimidine (1.25 g), 2-(11,11-dimethyl-11H-benzo[b]fluorene-4-yl)-4,4,5,5-tetramethyl-1,3,2-dioxaborolane (1.29 g), Pd₂(dba)₃ (0.05 g), and SPhos(0.10 g) were placed, and the atmosphere in the flask was replaced with argon gas. Then, 1,4-dioxane (29 mL) and an aqueous sodium carbonate solution (2M, 3.6 mL) were added thereto, and the mixture was refluxed with heat for 8 hours under stirring. After cooling the reaction solution, and distilling off the solvent from the reaction solution, the obtained crude product was purified by silica gel chromatography and washed with hexane to obtain ET-4 as white solids (1.72 g, yield: 93%).

As a result of the mass spectrum analysis, m/e=641 with respect to the molecular weight of 640.78, so that the product was identified as the desired compound.

### (Synthesis Example 5) Synthesis of ET-5

ET-5 was synthesized in accordance with the following synthetic route.

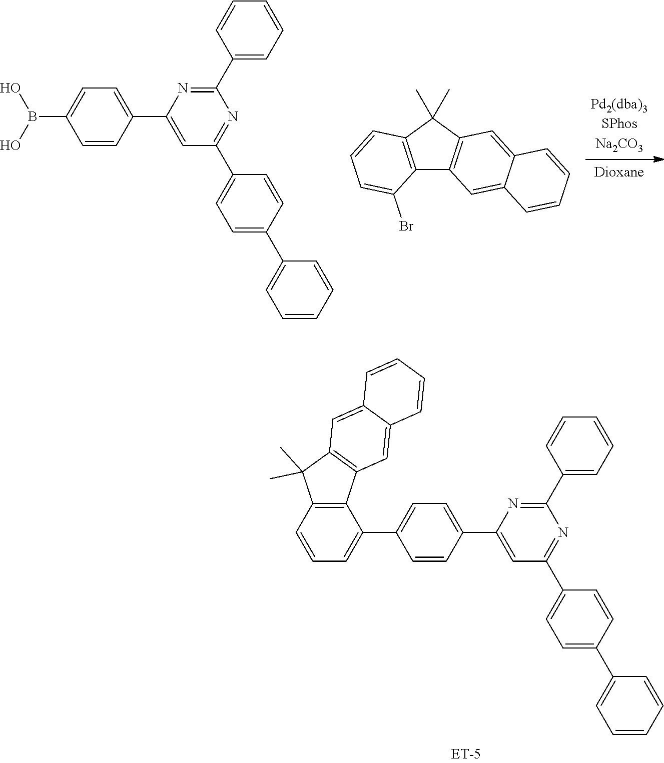 [SMILES: OB(O)c1ccc(-c2cc(-c3ccc(-c4ccccc4)cc3)nc(-c3ccccc3)n2)cc1 ; CC1(C)c2cc3ccccc3cc2-c2c(Br)cccc21 ; CC1(C)c2cc3ccccc3cc2-c2c(-c3ccc(-c4cc(-c5ccc(-c6ccccc6)cc5)nc(-c5ccccc5)n4)cc3)cccc21]

In a flask, (4-(6-([1,1′ biphenyl]-4-yl)-2-phenylpyrimidin-4-yl)phenyl)boronic acid (3.13 g), 4-bromo-11,11-dimethyl-11H-benzo[b]fluorene (2.36 g), Pd₂(dba)₃ (0.13 g), and SPhos(0.24 g) were placed, and the atmosphere in the flask was replaced with argon gas. Then, 1,4-dioxane (49 mL) and an aqueous sodium carbonate solution (2M, 18.2 mL) were added thereto, and the mixture was refluxed with heat for 8 hours under stirring. After the reaction solution was cooled, dichloromethane and water were added to the reaction solution, followed by liquid separation, and the oil phase was recovered. After distilling off the solvent, the obtained crude product was purified by silica gel chromatography and recrystallized from 1,2 dimethoxyethane to obtain ET-5 as white solids (2.69 g, yield: 59%).

As a result of the mass spectrum analysis, m/e=627 with respect to the molecular weight of 626.80, so that the product was identified as the desired compound.

### (Synthesis Example 6) Synthesis of ET-6

ET-6 was synthesized in accordance with the following synthetic route.

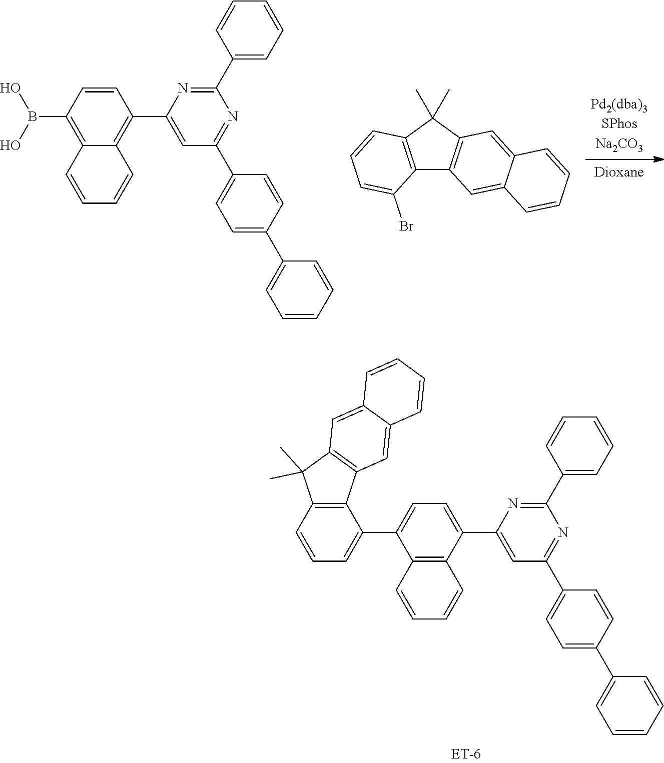 [SMILES: OB(O)c1ccc(-c2cc(-c3ccc(-c4ccccc4)cc3)nc(-c3ccccc3)n2)c2ccccc12 ; CC1(C)c2cc3ccccc3cc2-c2c(Br)cccc21 ; CC1(C)c2cc3ccccc3cc2-c2c(-c3ccc(-c4cc(-c5ccc(-c6ccccc6)cc5)nc(-c5ccccc5)n4)c4ccccc34)cccc21]

In a flask, (4-(6-([1,1′ biphenyl]-4-yl)-2-phenylpyrimidin-4-yl)naphthalene-1-yl)boronic acid (4.78 g), 4-bromo-11,11-dimethyl-11H-benzo[b]fluorene (3.23 g), Pd₂(dba)₃ (0.18 g), and SPhos (0.33 g) were placed, and the atmosphere in the flask was replaced with argon gas. Then, 1,4-dioxane (67 mL) and an aqueous sodium carbonate solution (2M, 25.0 mL) were added thereto, and the mixture was refluxed with heat for 6.5 hours under stirring. After the reaction solution was cooled, dichloromethane and water were added to the reaction solution, followed by liquid separation, and the oil phase was recovered. After distilling off the solvent, the obtained crude product was purified by silica gel chromatography and washed with toluene and ethanol to obtain ET-6 as white solids (5.98 g, yield: 88%).

As a result of the mass spectrum analysis, m/e=677 with respect to the molecular weight of 676.86, so that the product was identified as the desired compound.

### (Synthesis Example 7) Synthesis of ET-7

ET-7 was synthesized in accordance with the following synthetic route.

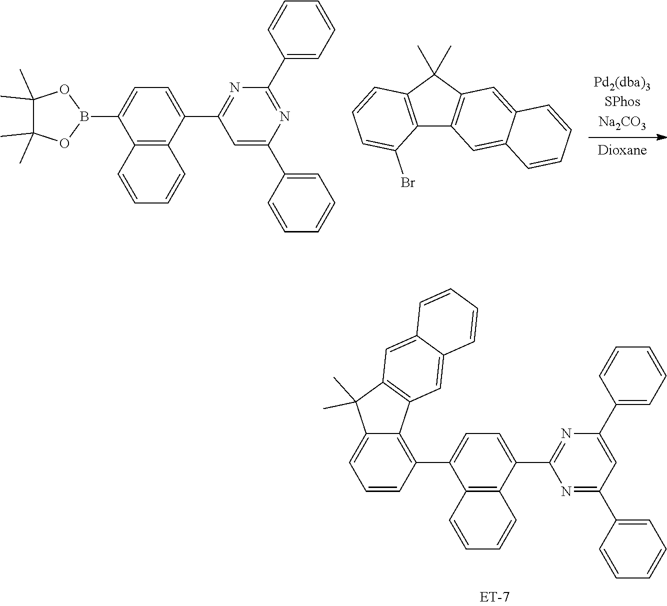 [SMILES: CC1(C)OB(c2ccc(-c3cc(-c4ccccc4)nc(-c4ccccc4)n3)c3ccccc23)OC1(C)C ; CC1(C)c2cc3ccccc3cc2-c2c(Br)cccc21 ; CC1(C)c2cc3ccccc3cc2-c2c(-c3ccc(-c4nc(-c5ccccc5)cc(-c5ccccc5)n4)c4ccccc34)cccc21]

In a flask, 4,6-Diphenyl-2-(4-(4,4,5,5-tetramethyl-1,3,2-dioxaborolan-2-yl)naphthalene-1-yl)pyrimidine (4.84 g), 4-bromo-11,11-dimethyl-11H-benzo[b]fluorene (3.23 g), Pd₂(dba)₃ (0.18 g), and SPhos(0.33 g) were placed, and the atmosphere in the flask was replaced with argon gas. Then, 1,4-dioxane (67 mL) and an aqueous sodium carbonate solution (2M, 25.0 mL) were added thereto, and the mixture was refluxed with heat for 6.5 hours under stirring. After the reaction solution was cooled, dichloromethane and water were added to the reaction solution, followed by liquid separation, and the oil phase was recovered. The solvent was distilled off and the crude product was purified by silica gel chromatography to obtain ET-7 as white solids (4.50 g, yield: 75%).

As a result of the mass spectrum analysis, m/e=601 with respect to the molecular weight of 600.76, so that the product was identified as the desired compound.

### (Synthesis Example 8) Synthesis of ET-8

ET-8 was synthesized in accordance with the following synthetic route.

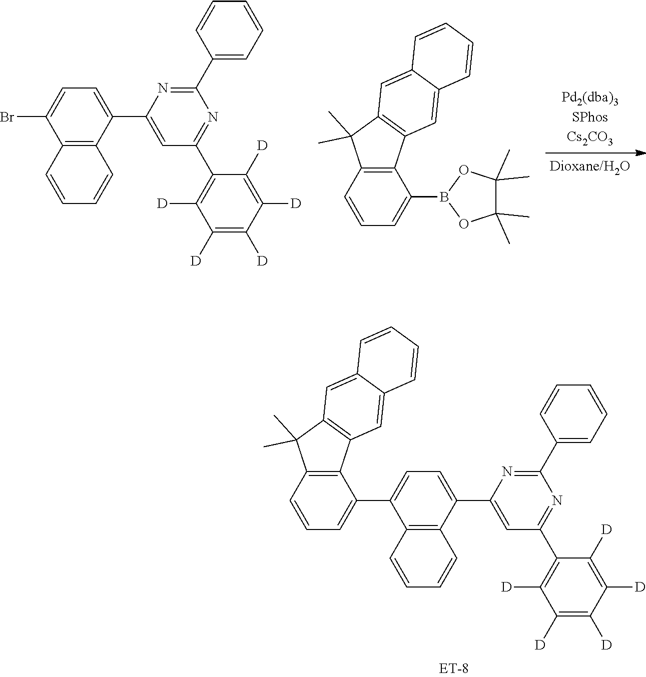

In a flask, 4-(4-Bromo-1-naphthalenyl)-2-phenyl-6-(phenyl-2,3,4,5,6-d5) pyrimidine (2.50 g), 4-bromo-11,11-dimethyl-11H-benzo[b]fluorene (1.65 g), Pd₂(dba)₃ (0.09 g), SPhos(0.17 g), and cesium carbonate (3.33 g) were placed, and the atmosphere in the flask was replaced with argon gas. Then, 1,4-dioxane (22 mL) and H₂O (3.7 mL) were added thereto, and the mixture was heated at 75° C. for 20 hours under stirring. After the reaction solution was cooled, the solvent was distilled off and the crude product was purified by silica gel chromatography and washed with hexane to obtain ET-8 as white solids (2.12 g, yield: 69%).

As a result of the mass spectrum analysis, m/e=606 with respect to the molecular weight of 605.79, so that the product was identified as the desired compound.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The documents described in the specification and the specification of Japanese application(s) on the basis of which the present application claims Paris convention priority are incorporated herein by reference in its entirety.

1. A compound represented by the following formula (1): 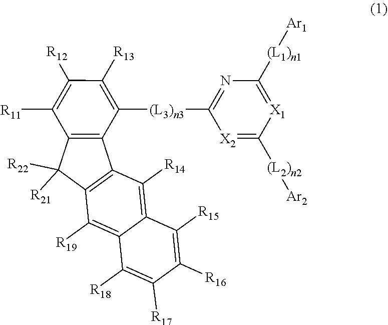
wherein in the formula (1),
X₁ and X₂ are independently N or CH, and any one of X₁ and X₂ is N,
Ar₁ and Ar₂ are independently
a substituted or unsubstituted aryl group including 6 to 50 ring carbon atoms, or
a substituted or unsubstituted monovalent heterocyclic group including 5 to 50 ring atoms,
L₁ to L₃ are independently
a single bond,
a substituted or unsubstituted arylene group including 6 to 50 ring carbon atoms, or
a substituted or unsubstituted divalent heterocyclic group including 5 to 50 ring atoms,
when n1 is an integer of 0 to 4 and when n1 is 0, (L₁)ₙ₁ is a single bond,
when a plurality of L₁'s is present, the plurality of L₁'s may be the same as or different from each other,
when n2 is an integer of 0 to 4 and when n2 is 0, (L₂)ₙ₂ is a single bond,
when a plurality of L₂'s is present, the plurality of L₂'s may be the same as or different from each other,
when n3 is an integer of 0 to 4 and when n3 is 0, (L₃)ₙ₃ is a single bond,
when a plurality of L₃'s is present, the plurality of L₃'s may be the same as or different from each other,
R₁₁ to R₁₉ are independently a hydrogen atom or a substituent R,
a set of adjacent two or more of R₁₁ to R₁₉ does not bond with each other,
R₂₁ and R₂₂ form a substituted or unsubstituted saturated or unsaturated ring by bonding with each other or do not form a substituted or unsubstituted saturated or unsaturated ring,
R₂₁ and R₂₂ which do not form a substituted or unsubstituted saturated or unsaturated ring are independently a hydrogen atom, or a substituted or unsubstituted alkyl group including 1 to 50 carbon atoms,
the substituent R is selected from the group consisting of
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, and
a substituted or unsubstituted monovalent heterocyclic group including 5 to 50 ring atoms,
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms,
when two or more of each of R₉₀₁ to R₉₀₇ are present, the two or more of each of R₉₀₁ to R₉₀₇ may be the same as or different from each other, and
when two or more substituent R's are present, the two or more substituent R's may be the same as or different from each other.

2. The compound according to claim 1, wherein the compound represented by the formula (1) is a compound represented by the following formula (11): 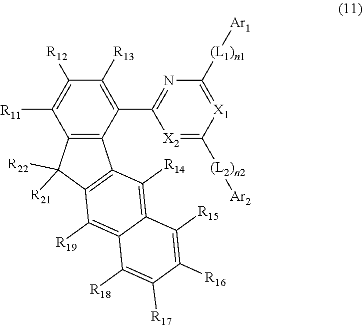
wherein in the formula (11),
X₁, X₂, Ar₁, Ar₂, L₁, L₂, n1, n2, R₁₁ to R₁₉, R₂₁, and R₂₂ are as defined in the formula (1).

3. (canceled)

4. The compound according to claim 1, wherein R₂₁ and R₂₂ are substituted or unsubstituted alkyl groups including 1 to 5 carbon atoms.

5. (canceled)

6. The compound according to claim 1, wherein X₁ is N and X₂ is CH.

7. The compound according to claim 2, wherein the compound represented by the formula (11) is a compound represented by the following formula (111) or (112): 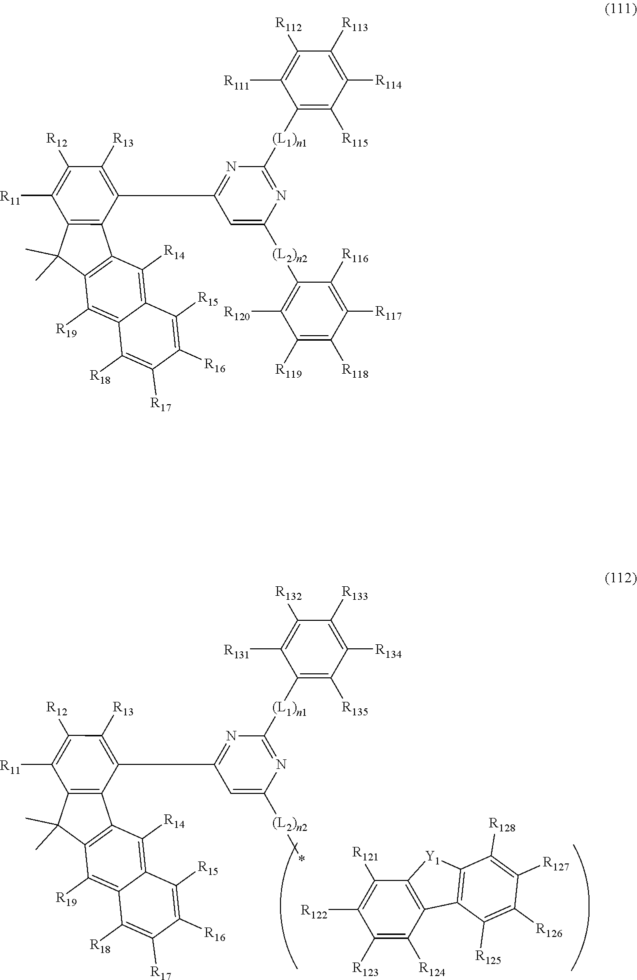
wherein in the formula (111),
L₁, L₂, n1, n2, and R₁₁ to R₁₉ are as defined in the formula (11),
one or more sets of adjacent two or more of R₁₁₁ to R₁₁₅ form a substituted or unsubstituted saturated or unsaturated ring by bonding with each other or do not bond with each other,
R₁₁₁ to R₁₁₅ which do not form a substituted or unsubstituted saturated or unsaturated ring are independently a hydrogen atom or a substituent R,
one or more sets of adjacent two or more of R₁₁₆ to R₁₂₀ form a substituted or unsubstituted saturated or unsaturated ring by bonding with each other or do not bond with each other, and
R₁₁₆ to R₁₂₀ which do not form a substituted or unsubstituted saturated or unsaturated ring are independently a hydrogen atom or a substituent R;
wherein in the formula (112),
L₁, L₂, n1, n2, and R₁₁ to R₁₉ are as defined in the formula (11),
Y₁ is N(R₁₂₉), C(R₁₃₀ₐ)(R_{130b}), O, or S,
one or more sets of adjacent two or more of R₁₂₁ to R₁₂₉ form a substituted or unsubstituted saturated or unsaturated ring by bonding with each other or do not bond with each other,
(L₂)ₙ₂ bonds any one of bondable positions of a fused ring structure in parentheses,
R₁₂₁ to R₁₂₉ which do not bond with each other and do not bond with (L₂)ₙ₂ are independently a hydrogen atom or a substituent R,
R₁₃₀ₐ and R_{130b} form a substituted or unsubstituted saturated or unsaturated ring by bonding with each other or do not form a substituted or unsubstituted saturated or unsaturated ring by bonding with each other,
R₁₃₀ₐ and R_{130b} which do not form a substituted or unsubstituted saturated or unsaturated ring are independently a hydrogen atom, or a substituent R,
one or more sets of adjacent two or more of R₁₃₁ to R₁₃₅ form a substituted or unsubstituted saturated or unsaturated ring by bonding with each other or do not bond with each other,
R₁₃₁ to R₁₃₅ which do not form a substituted or unsubstituted saturated or unsaturated ring are independently a hydrogen atom or a substituent R, and
the substituent R is as defined in the formula (411).

8. (canceled)

9. The compound according to claim 1, wherein X₁ is CH and X₂ is N.

10. The compound according to claim 1, wherein the compound represented by the formula (1) is a compound represented by the following formula (21): 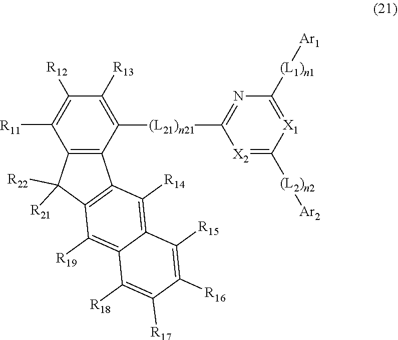
wherein in the formula (21),
X₁, X₂, Ar₁, Ar₂, L₁, L₂, n1, n2, R₁₁ to R₁₉, R₂₁, and R₂₂ are as defined in the formula (1),
L₂₁ is
a substituted or unsubstituted arylene group including 6 to 50 ring carbon atoms, or
a substituted or unsubstituted divalent heterocyclic group including 5 to 50 ring atoms,
n21 is an integer of 1 to 4, and
when a plurality of L₂₁'s is present, the plurality of L₂₁'s may be the same as or different from each other.

11. (canceled)

12. The compound according to claim 10, wherein R₂₁ and R₂₂ are substituted or unsubstituted alkyl groups including 1 to 5 carbon atoms.

13. (canceled)

14. The compound according to claim 10, wherein L₂₁ is a substituted or unsubstituted arylene group including 6 to 50 ring carbon atoms.

15. (canceled)

16. The compound according to claim 10, wherein X₁ is N and X₂ is CH.

17. The compound according to claim 10, wherein the compound represented by the formula (21) is a compound represented by any one of the following formulas (211) to (213): 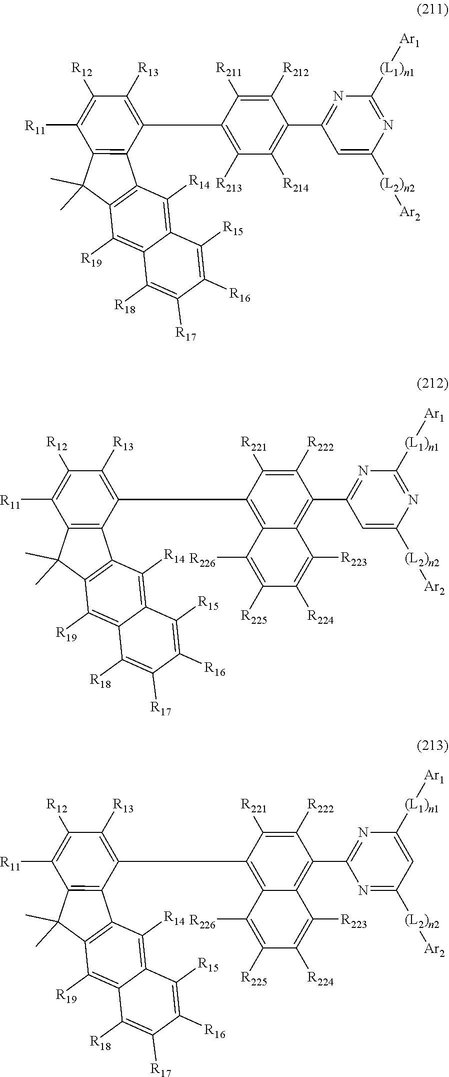
wherein in the formula (211),
Ar₁, Ar₂, L₁, L₂, n1, n2, and R₁₁ to R₁₉ are as defined in the formula (421),
R₂₁₁ to R₂₁₄ are independently a hydrogen atom or a substituent R, and
one or more sets of adjacent two or more of R₂₁₁ to R₂₁₄ do not bond with each other;
wherein in the formula (212) and (213),
Ar₁, Ar₂, L₁, L₂, n1, n2, and R₁₁ to R₁₉ are as defined in the formula (421),
R₂₂₁ to R₂₂₆ are independently a hydrogen atom or a substituent R,
one or more sets of adjacent two or more of R₂₂₁ to R₂₂₆ do not bond with each other, and
the substituent R is as defined in the formula (21).

18. (canceled)

19. The compound according to claim 10, wherein the compound represented by the formula (21) is a compound represented by the formula (221) or the formula (222): 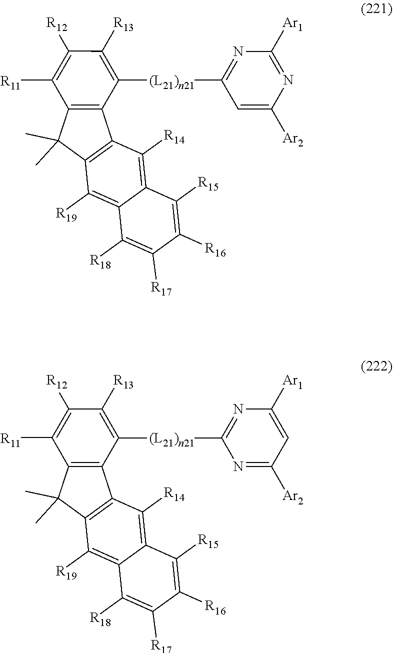
wherein in the formula (221) and formula (222),
Ar₁, Ar₂, R₁₁ to R₁₉, L₂₁, and n21 are as defined in the formula (21).

20. The compound according to claim 1, wherein L₁ and L₂ are independently a single bond, a substituted or unsubstituted phenylene group, or a substituted or unsubstituted naphthylene group.

21. The compound according to claim 1, wherein Ar₁ and Ar₂ are independently a substituted or unsubstituted aryl group including 6 to 18 ring carbon atoms, or a substituted or unsubstituted monovalent heterocyclic group including 5 to 18 ring atoms.

22. The compound claim 1, wherein Ar₁ and Ar₂ are independently a substituted or unsubstituted phenyl group, a substituted or unsubstituted naphthyl group, or a substituted or unsubstituted biphenyl group.

23. The compound according to claim 1, wherein R₁₁ to R₁₉ are hydrogen atoms.

24-26. (canceled)

27. An organic electroluminescence device comprising:
a cathode,
an anode, and
one or two or more organic layers arranged between the cathode and the anode,
wherein at least one of the organic layers comprises the compound according to claim 1.

28. The organic electroluminescence device according to claim 27, which comprises an anode, an emitting layer, an electron-transporting region, and a cathode in this order,
wherein electron-transporting region comprises the compound.

29. The organic electroluminescence device according to claim 28, wherein the electron-transporting region comprises a first layer and a second layer in this order from the emitting layer side, and
the first layer comprises the compound.

30. An electronic apparatus equipped with the organic electroluminescence device according to claim 27.
